(19) United States
(12) Patent Application Publication
(10) Pub. No.: US 2003/0032779 A1
(43) Pub. Date: Feb. 13, 2003

(54) Estrogen receptor ligands

(76) Inventors: Lars Ohman, Stockholm (SE); Tomas Bonn, Huddinge (SE); Mats Carlquist, Spanga (SE); Owe Engstrom, Nacka (SE); Patrick Goede, Tumba (SE); Asa Hedfors, Huddinge (SE); Erik Holmgren, Gutavsberg (SE); Konrad F. Koehler, Huddinge (SE); Andrzej M. Brzozowski, York (GB); Ashley C.W. Pike, York (GB); Roderick E. Hubbard, York (GB)

Correspondence Address: WIGGIN & DANA LLP, ATTENTION: PATENT DOCKETING, ONE CENTURY TOWER, P.O. BOX 1832, NEW HAVEN, CT, 06508-1832, US

(21) Appl. No.: 10/163,430

(22) Filed: Jun. 5, 2002

Related U.S. Application Data
(62) Division of application No. 09/445,265, filed on Jun. 23, 2000.
Application No. 09/445,265 is a 371 of international application No. PCT/GB98/01708, filed on Jun. 10, 1998.

(30) Foreign Application Priority Data
Jun. 10, 1997 (GB) 9711992.9
Aug. 15, 1997 (GB) 9717346.2
Feb. 13, 1998 (GB) 9803061.2
Feb. 16, 1998 (GB) 9803202.2

(51) Int. Cl. C07K 14/715

(52) U.S. Cl. 530/350

(57) ABSTRACT

The present invention is directed to crystals comprising at least part of the estrogen receptor α (ERα) ligand binding domain, optionally bound to a ligand. The present invention is also directed to ligands that bind to ER receptors, and method of designing them. The present invention is also directed to a homology model of the ERβ receptor.

This invention relates to estrogen receptor ligands. More particularly, the present invention relates to ligands which will bind to estrogen receptors, crystals of such receptors, including crystals of receptor and ligand, synthetic ligands, methods of using such synthetic ligands and methods for designing ligands which will bind to the estrogen receptor.

The thyroid hormone receptor (TR) is known and its three-dimensional structure, and hence its ligand binding domain, has been determined. Knowledge of the three-dimensional structure has enabled a better understanding of the modes of ligand binding and the determination of the optimum conformation of ligand to bind to the receptor. This understanding will provide a pharmacophore model usable in the design of ligands, such as drugs, to bind to the thyroid receptor. It is generally believed in the art that the TR structure also provides a guide to the design of ER ligands.

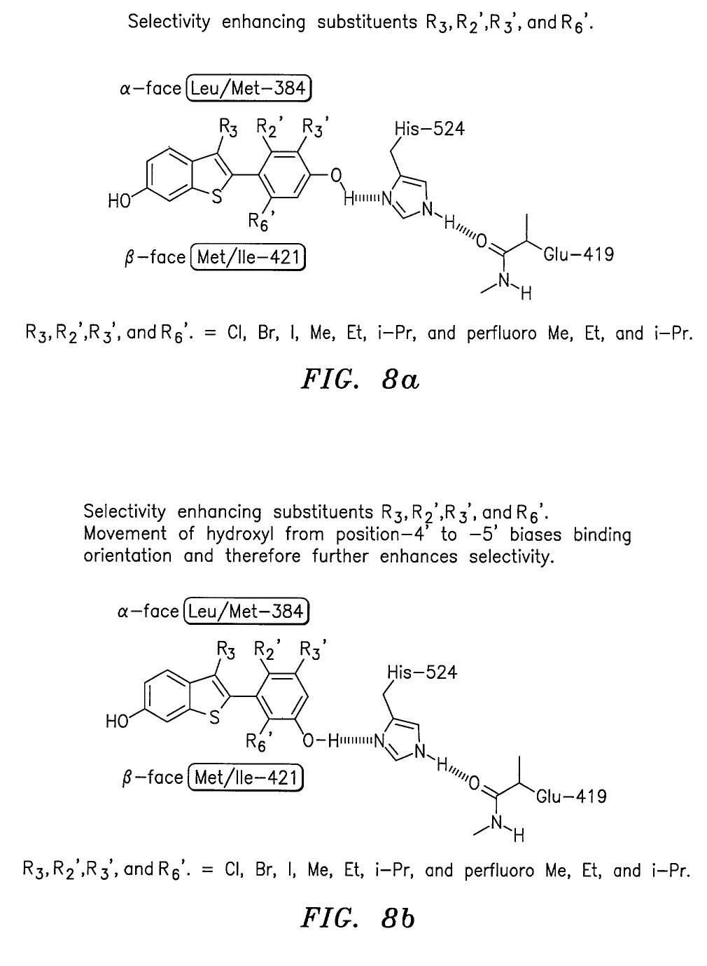

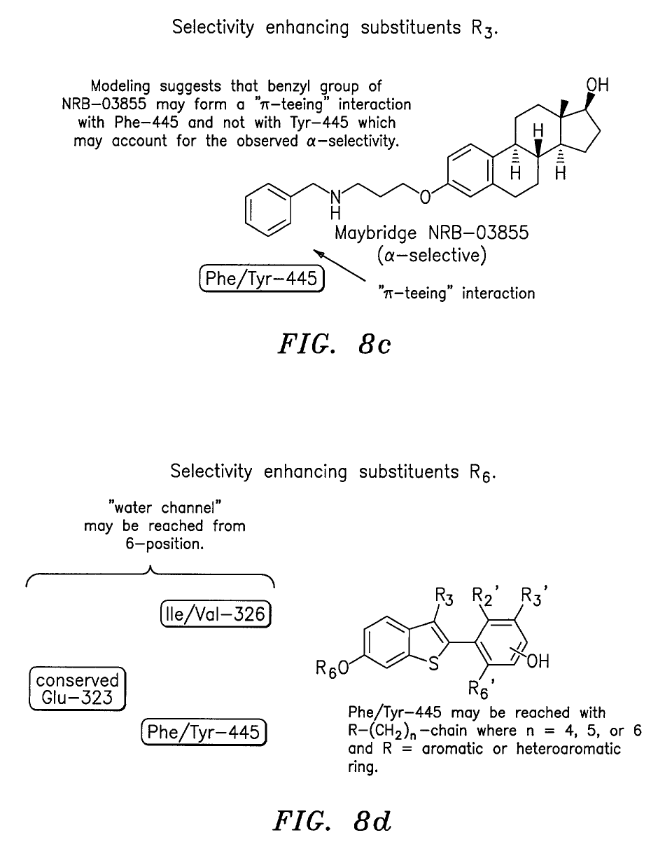

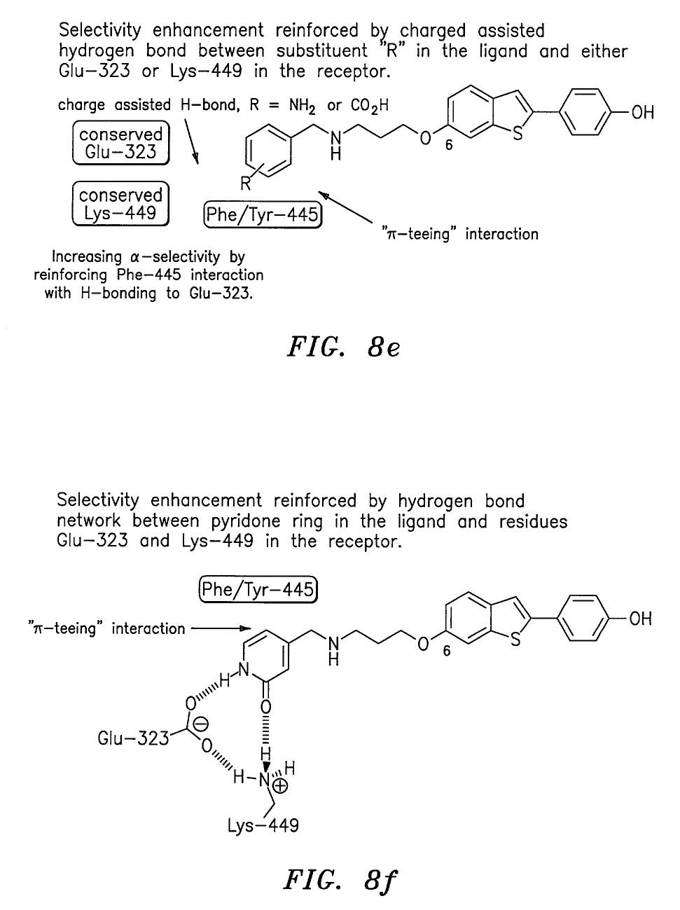

Estrogen steroid hormone and thus the estrogen receptor (ER) is a member of the steroid hormone receptor family. Its primary natural ligand is estradiol (E2). However, it is known that a large number of structurally diverse non-steroidal compounds such as raloxifene, centchroman, coumestrol, diethylstilbesterol, esculin, tamoxifen, zearalenone, and zindoxifen also bind to the estrogen receptor (FIG. 8). The majority of these non-steroidal estrogen receptor ligands contain 2-4 carboxyclic, aromatic, and/or heterocyclic rings connected by a 1-3 atom chain. One or more of the rings may be fused with the central atom chain or with each other.

It has been proposed that the receptor possesses a multi-functional modular structure potentially having discrete domains for DNA binding, ligand binding, and transactivation. The ligand binding domain (LDB) has been designated domain E and is the largest domain of the estrogen receptor. The ligand binding domain includes a ligand recognition site and regions for receptor dimerzation, interaction with heat shock proteins, nuclear localization and ligand dependent transactivation.

A review of the structure and functioning of the estrogen receptor is provided in an article by Katzenellenbogen, J. et al., Steroids, (1997) 62(3): 268-303.

It is known that compounds which bind to the estrogen receptor are potentially useful in the treatment a wide range of disease states. These include estrogen agonists for treatment of disease linked to estrogen deficiency (e.g., osteoporosis, cardiovascular and neurodegenerative diseases in post menopausal women) and estrogen antagonists for treatment of breast and uterine cancer. Furthermore, it is known that certain ligands such as tamoxifen display mixed agonist/antagonist action (that is they are either estrogen agonists, estrogen antagonists, or a partial estrogen antagonists when binding to the estrogen receptors of different tissues) and such compounds may simultaneously prevent bone loss and reduce the risk of breast cancer. It is further known that benzothiophenes are usable as agonists or antagonists to steroid hormones, and that it is possible to modify their binding mechanics, for example the binding affinity, by changing the substituent groups at various positions on the molecule. Therefore, it would be desirable to be able to design ligands which are recognizable by and able to bind to the estrogen receptor. Additionally, it would be desirable to know the three dimensional structure of the estrogen receptor. Such knowledge would be useful for the design of compounds intended to bind to the estrogen receptor. The present inventors have been able to produce an estrogen receptor crystal and to determine from that the three dimensional structure of the estrogen receptor. Unexpectedly, the thus determined ER structure reveals that the TR structure does not provide a good model for binding of ligands to ER.

Therefore, in a first aspect the present invention provides an estrogen receptor ligand binding domain crystal.

In a second aspect, the present invention provides ligands, particularly synthetic ligands, of estrogen receptors by use of the crystals.

In a third aspect of the invention, methods for designing ligands which will bind to the estrogen receptor are provided. Such methods use three dimensional models based on the crystals of the estrogen receptor. Generally, such methods comprise determining compounds which are likely to bind to the receptor based on their three dimensional shape compared to that of the estrogen receptor and in particular the ligand binding domain of the estrogen receptor. Preferably, those compounds have a structure which is complementary to that of the estrogen receptor. Such methods comprise the steps of determining which amino acid or amino acids of the ligand binding domain of the estrogen receptor interacts with the binding ligand, and selecting compounds or modifying existing compounds, to improve the interaction. Preferably, improvements in the interaction are manifested as increases in the binding affinity but may also include increases receptor selectivity and/or modulation of efficacy.

Preferably, the ligands bind to the ER with a high binding affinity, for example within the range of 20-2000 pmol.

The ligands may bind tightly bind to the ER yet not up-regulate gene expression thereby inhibiting the action of estradiol and estradiol mimetics. Thus, the invention also provides a method of inhibiting the activity of estradiol or estradiol mimetics by providing ligands which bind to ER with a high affinity, blocking the activity of estrogens. Alternatively, binding of the ligand to the ER may cause conformational changes to the ER inhibiting further binding thereto. The invention further provides a method of inhibit estradiol activity in an animal, the method comprising administering to the animal a ligand which binds to at least the LBD, of the ER with high affinity and blocks binding of further ligands to at least the LDB of the ER. Such ligands are useful in, for example, the treatment of estrogen receptor mediated diseases in females.

Structure Based Design of ER Ligands

The present work has elucidated the structure of the ligand binding cavity of the estrogen receptor. Knowledge of the structure of this cavity has utility in the design of structurally novel ER ligands and in the design of non-obvious analogs of known ER ligands with improved properties. These enhanced properties include one or more of the following: (1) higher affinity, (2) improved selectivity for either the α- or β-isoform of the ER, and/or (3) a designed degree of efficacy (agonism vs. partial agonism vs. antagonism). Without knowledge of the ER structure, modifications to produce ligands with enhanced properties and a reasonable likelihood of success would not be available to those skilled in the art. The ER receptor structure also has utility in the discovery of new, structurally novel classes of ER ligands. Electronic screening of large, structurally diverse compound libraries such as the Available Chemical Directory (ACD) will identify new structural classes of ER ligands which will bind to the 3-dimensional structure of the estrogen receptor. Additionally the ER structure allows for “reverse-engineering” or “de novo design” of compounds to bind to the ER.

(1) Enhanced Affinity

The present work has revealed the presence of receptor defined β- and α-face cavities centered respectively above and below the B- and C-rings of estradiol.

The present invention provides new ligands which exploit this discovery by filling the α- and β-face cavities.

Preferably, the ligand fills at least one of the α- and β-face cavities so as to exclude water from the cavity or cavities.

The ligands produced in accordance with the invention bind more effectively to the ER than estradiol. The ligand may bind with twice the binding affinity of estradiol, preferably three times the affinity, and most preferably ten or more times the affinity.

Modifications to the steroid nucleus may be made at the positions marked in R in FIG. 8a and 8b (α-substitution at the 7-, 9-, 12-, 14-, 16-, and 17-positions; β-substitution at the 8-, 11-, 15-, and 18-positions). Preferably, those substituents are hydrophobic substituents, e.g., methyl, ethyl, iso-propyl, chlorine, bromine, or iodine.

Modifications to 2-aryl benzothiophenes may be made at the 2′-, 3′-, and 6′-positions (FIG. 8c) in order to fill the α- and β-face cavities of ER. Preferably substituents should be present in at least two of the following three positions: 3, 2′, or 6′ so that a perpendicular conformation between the B- and C-rings of the 2-aryl benzothiophene nucleus is enforced. This perpendicular conformation facilitates the positioning of the 2′-, 3′-, and 6′-substituents in the α- and β-face cavities of ER.

In a manner analogous to the benzothiophene series, the affinity of other classes of non-steroidal ER ligands may be enhanced by substitution of small hydrophobic substituents at positions marked R2′, R3′, and/or R6′ in FIG. 8C.

Preferably, the ligand produce in accordance with the invention fills at least one of the α- and β-cavities of the ER without perturbing the remainder of the ER structure.

Another aspect of this invention reveals an unfilled hydrophobic cavity in the raloxifene/ER complex. Filling this cavity with hydrophobic substituents so as to exclude water will enhance binding affinity. This cavity may be filled by positioning a hydrophobic substituent on the ethoxyphenyl sidechain a to the piperidinyl nitrogen atom of raloxifene. This hydrophobic substituent may be a linear alkyl or perfluoroalkyl group (—CH₃ to —C₁₀H₂₁, —CF₃ to —C₁₀F₂₁), benzyl (—CH₂Ph, or methylene cyclohexyl (—CH₂C₆H₁₁).

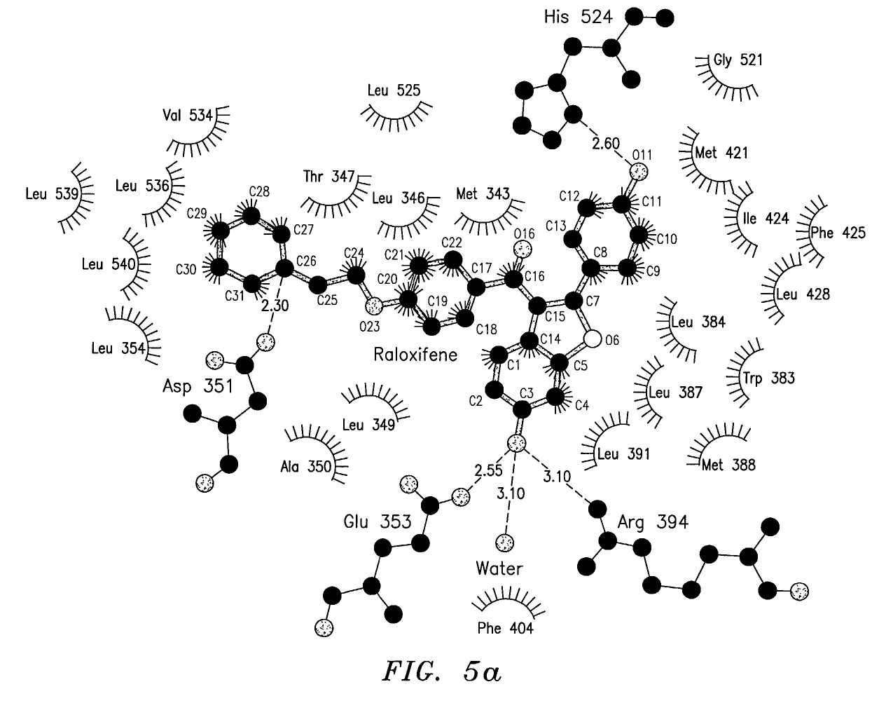

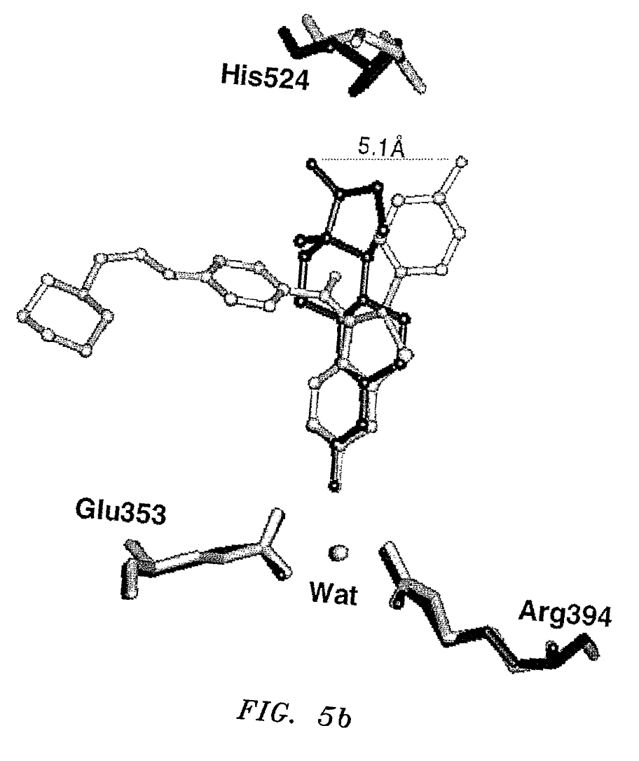

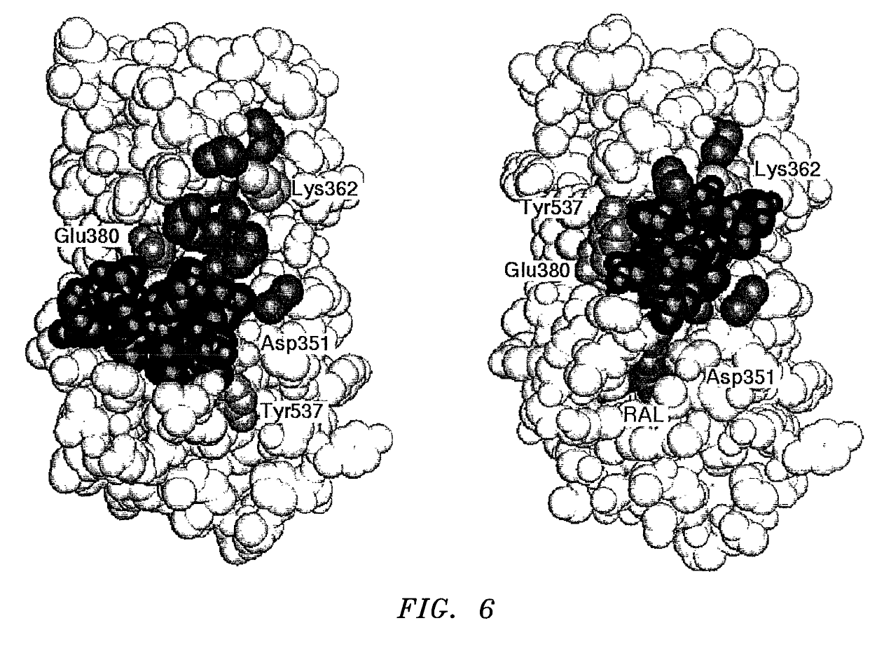

In a third aspect of this invention, examination of the ER structure reveals that the hydroxyl group at position-3 of estradiol or position-6 of raloxifene form hydrogen bonding interactions with Glu-353 and Arg-394 (FIGS. 5a and 5b). It is known that replacement of the hydroxyl group at position-3 of estradiol or position-6 of raloxifene results in a decrease in affinity for the ER. The invention reveals the reason for this reduction in affinity: while one of the hydrogen atoms of the amino group forms a favorable hydrogen bonding interaction with Glu-353, the second hydrogen atom forms an unfavorable electrostatic interaction with Arg-394. Furthermore this invention reveals a method for enhancing the affinity of 3-amino analogs of estradiol and 6-amino analogs of raloxifene: replacement of one of the two hydrogen atoms of the amino group with an alkyl group to produce a secondary amino group. Alternatively, the amino group may be replaced with a guanidino group (FIG. 8e) which will pick up two additional hydrogen bonding interactions, the first is a salt bridge to Glu-353 and the second is a hydrogen bonding interaction with a backbone carbonyl group in residue Leu-387. Similar enhancement of affinity for the ER may be achieved by replacement of the guanidino group with a fused 2-aminopyrrole (FIG. 8e).

In a closely related aspect of this invention, an understanding is provided for the reduction in affinity for the ER seen in ether derivatives at either position-3 of estradiol or position-6 of raloxifene: electrostatic repulsion between the ether oxygen atom of the ligand and Glu-353 in the ER. This invention reveals a way of increasing the affinity of estradiol position-3 or raloxifene position-6 ether derivatives: replacement of the ether oxygen atom with an amino (NH) group.

In a fourth aspect of this invention, replacement of the 4-hydroxyl group of raloxifene will enhance affinity by picking up a second hydrogen bonding interaction between the amino group and a backbone carbonyl group in Gly-521 of the ER (FIG. 8d).

(2) Improved Selectivity

The estrogen receptor has been found to have two discrete forms, known as ERα and ERβ. Furthermore the ratio of the α- to the β-forms of the ER may vary considerably in different cell and tissue types. Therefore it may be possible to dissociate desirable therapeutic effects from undesirable side effects of estrogen receptor ligands by designing ligands that selectively bind to one or the other isoforms of the estrogen receptor.

The α- and β-forms of the estrogen receptor differ significantly in their primary sequence and slightly in their tertiary structure. As a consequence of these receptor differences, ligands may bind with different affinity to the two isoforms.

The present inventors have been able to isolate, differentiate and produce crystals for the ERα. From these crystals, the present inventors have determined the three dimensional structure to high resolution. Further, the inventors have created a partial homology model of ERβ based on the experimentally derived ERα coordinates. This partial ERβ homology model captures the essential differences in binding properties between ERα and ERβ. Based on a comparison of the experimental ERα coordinates and the partial homology model of the ERβ, the differences between the ERα and ERβ have been determined and using these differences, the ability of a ligand to bind to either the ERα and ERβ receptors or to both receptors can be predicted. Hence, if it is known that one tissue possesses solely one form of the estrogen receptor, then it is possible to confer a degree of tissue specificity to a ligand by designing the ligand to bind to that predominant form of the receptor. Advantageously, the ligands may be designed to specifically bind ERα ir ERβ.

Furthermore, a detailed understanding of the different receptors enables the different behavior of a compound in different tissues to be understood, for example the estrogenic or anti-estrogenic behavior of raloxifen (RAL) dependence on the tissue in which it is active.

Thus, in a further aspect, the invention provides estrogen receptor ligand binding domain crystals for ERα and a partial homology model for ERβ. Specificity of ligands for either the ERα and ERβ or even to a specific ratio of ERα to ERβ is also provided. The advantage of this is that tissue specificity is conferred to the ligand. Thus, the invention also provides ligands, particularly synthetic ligands of ERα and ERβ together with methods for their design.

The present invention provides new ligands which exploit these differences by positioning ligand substituents in close proximity to one or more amino acid residue that differ between the α- and β-isoforms of the ER.

The ligands produced in accordance with the invention bind more effectively to either the α- or β-isoforms of the ER. The selectivity of the binding between the α- or β-isoforms may be ten-fold, more preferably one hundred-fold, and most preferably greater than one thousand-fold.

For example, in the β-face cavity of ER-α, the amino acid residue at position-384 is Leu (sidechain volume=76.6 Å) whereas in the corresponding position of ER-β, the amino acid residue is Met (sidechain volume=79.3 Å³). Therefore the β-face cavity of ER-β is smaller. Consequently ER-α selectivity may be enhanced by positioning substituents larger than a methyl group in the β-face cavity in close proximity to residue-384. Interaction between the ligand and residue-384 may be enhanced by introducing substituents at the β 8-, 15-, or 18-positions on the steroid nucleus.

In the α-face cavity of ER-α, the amino acid residue at position-421 is Met (sidechain volume=79.3 Å³) whereas in ERβ, it is Ile (sidechain volume=77.3 Å³). Therefore the α-face cavity of ER-α is smaller. This difference may be exploited to produce β-selective compounds through substitutions larger than a methyl group at the α 14-, 16-, or 17-positions of the steroid nucleus.

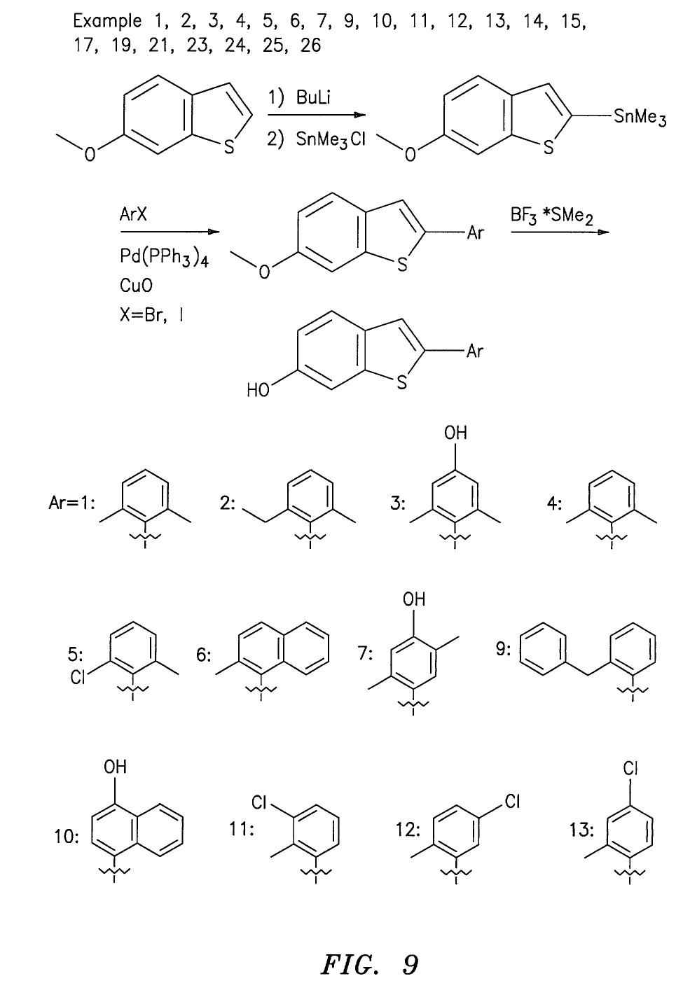

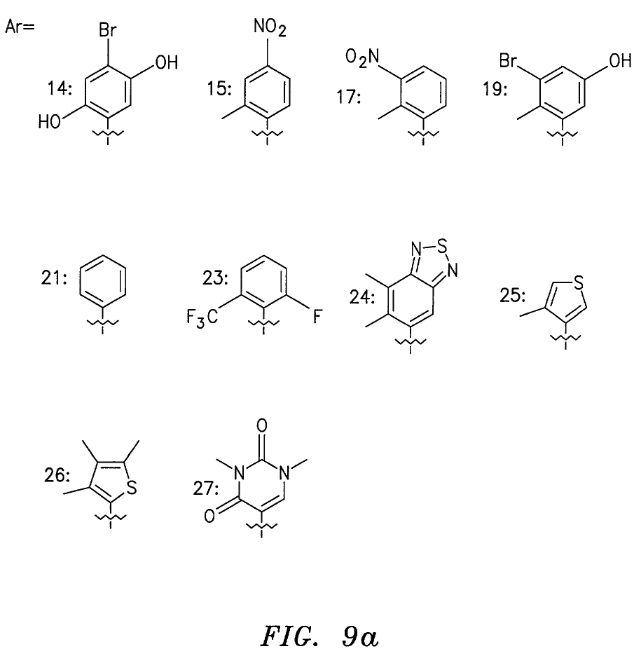

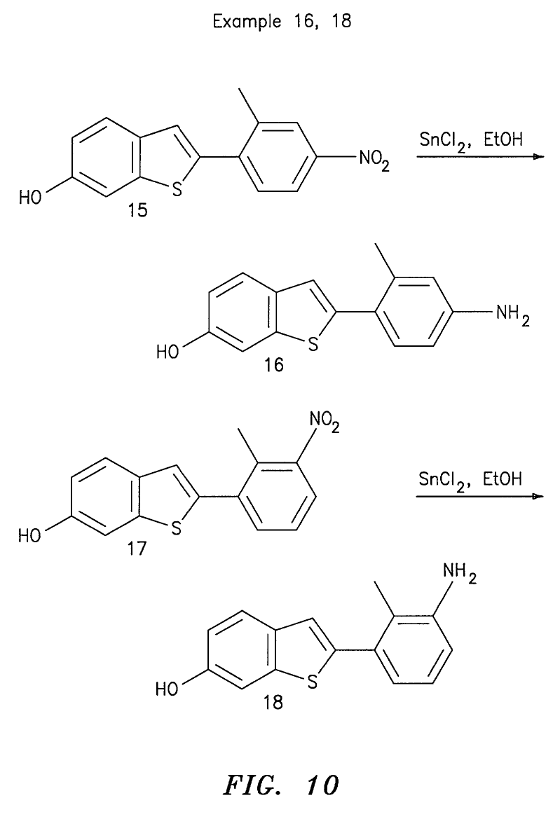

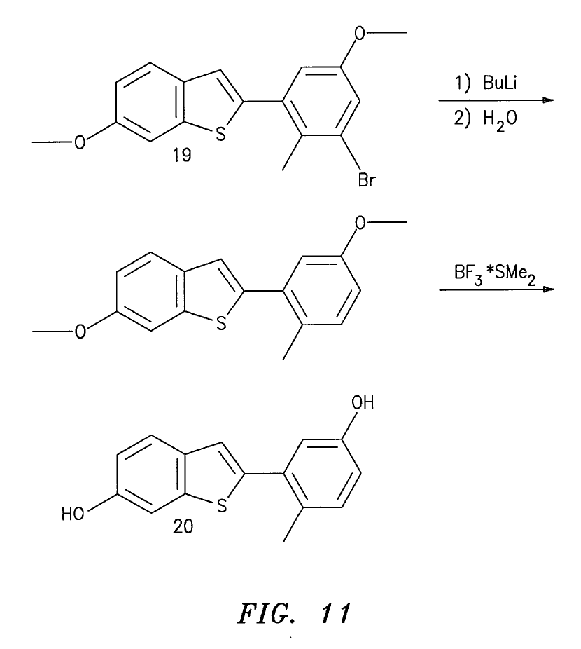

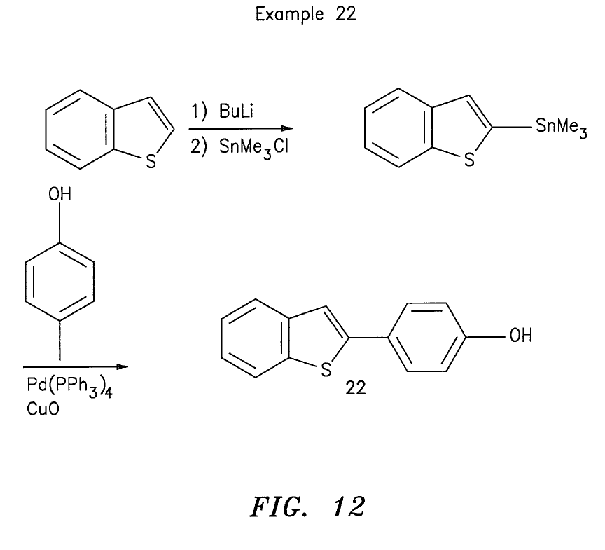

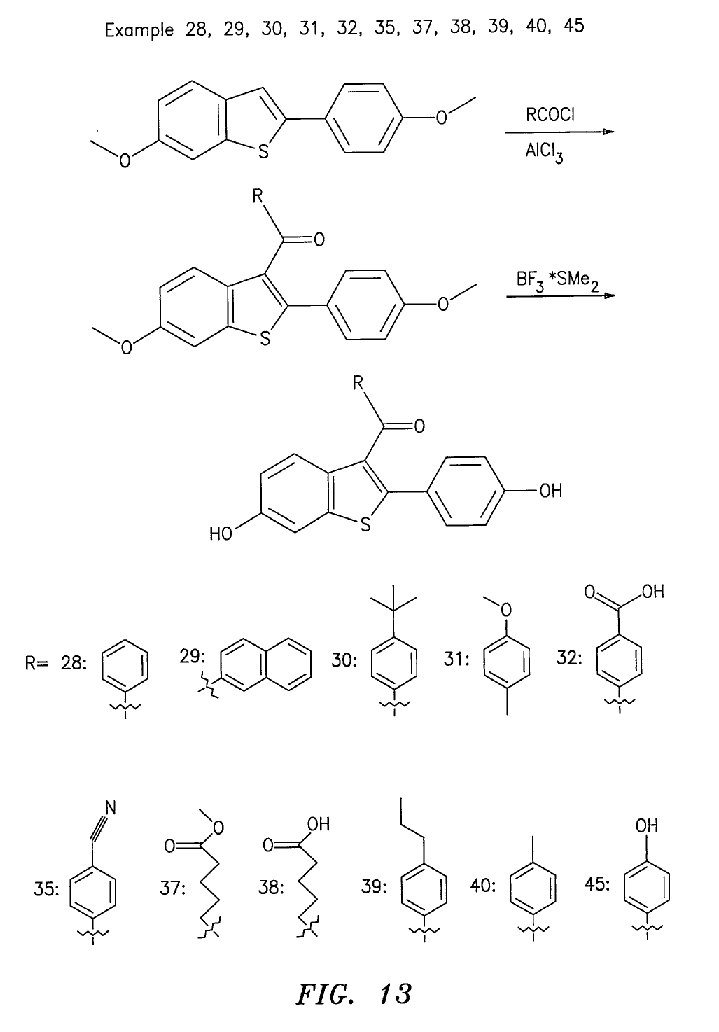

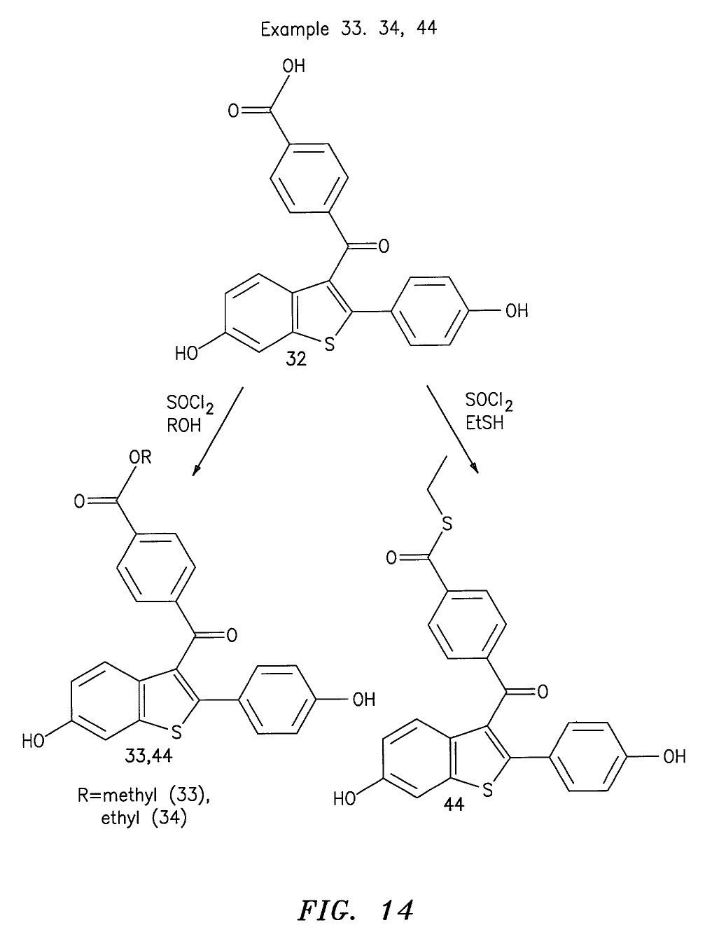

Similarly, substitutions may be made from either the 2′- or 3′-positions of the 2-arylbenzothiophene nucleus to interact with residue-384 in the β-face cavity or from the 6′-position to interact with residue-421 in the α-face cavity (FIGS. 9a and 9b). However free rotation about the C2—C1′ bond will effectively interchange the substituents at the 2′- and 6′-positions thereby reducing selectivity. Moving the hydroxyl group from position-4′ (FIG. 9a) to position-5′ (FIG. 9b) will bias the binding orientation such that the R₂ substituent will be positioned in the P-face pocket and the R₆ substituent in the α-face pocket. This bias results from the fact that only one of the two possible rotamers about the C2′C1′ bond will allow hydrogen bond formation between the 5′-hydroxyl group and the receptor residue His-524.

This invention also provides a means of enhancing the selectivity of other classes of non-steroidal ER ligands. In a manner analogous to the benzothiophene series of ER ligands, substituents larger than methyl may be introduced at either the R2′ or R3′ positions to produced ER-α selective compounds or at R₆′ to produce ER-β selective compounds (FIG. 8c).

Substitutions may be made from position-3 of the steroid nucleus or position-6 of the benzothiophene nucleus to exploit the differences between ER-α and ER-β at position-326 (Ile in ER-α and Val in ER-β) and at position-445 (Phe in ER-α and Tyr in ER-β).

This invention also provides a means for producing specifically ER-α selective ligands. A six atom linker between the hydroxyl group at position-3 of the A-ring of estradiol or at position-6 raloxifene and an aromatic or heteroaromatic ring on the sidechain will position the sidechain ring in close proximity to residue-445 (FIG. 9c). The edge of ER-α Phe-445 and the face of the sidechain ring can form a favorable “π-teeing” interaction. This favorable interaction is not possible with the ER-β Tyr-445, therefore analogs of this type with be ER-α selective (FIG. 9d).

Another aspect of this invention provides a means of further enhancing ER-α selectivity. Introduction of a carboxylate or amino group on the meta or para position of the above mentioned aromatic or heteraromatic ring will form a hydrogen bonding interaction between the conserved Glu-323 or Lys-449 (FIG. 9e). Alternatively, the heteroaromatic ring may be a pyridone ring which will simultaneously form favorable hydrogen bonding interactions with both Glu-323 or Lys-449 (FIG. 9f). Either of the amino, carboxylate, or pyridone ring substitutions will reinforce the favorable “π-teeing” interaction between the aromatic or heteroatomic ring of the ligand and Phe-445 in ER-α.

(3) Modulation of Efficacy

This invention provides an understanding of the differences between estrogen and antiestrogen binding and therefore a means to design ER ligands with the desired degree of efficacy. An examination of the differences between the ER/estradiol and ER/raloxifene complexes reveals a large movement in Helix-12 (H12, FIG. 6). H12 adopts an “agonistic” conformation defined by the structure of the ER/estradiol complex and an “antagonistic” conformation defined by the structure of the ER/raloxifene complex. These two conformation are in thermodynamic equilibrium. When the ER is complexed with a full agonist, such as estradiol, the equilibrium lies far in the direction of the “agonistic” conformation. In contrast, while when complexed with an antagonist, the equilibrium is pushed in the direction of the “antagonistic” conformation. In the case of raloxifene, the large sidechain at position-3 sterically collides with H12 in it's agonistic conformation, thereby driving the equilibrium strongly in the antagonistic direction. By introduction of progressively shorter sidechains at position-3 of raloxifene, the equilibrium will be gradually shifted back towards the agonist conformation. Thus, this invention provides a means of developing ligands with the desired degree of efficacy (agonist, partial agonist, or antagonist).

In particular, the importance of H12 has been determined as playing a central role in determining the efficacy (agonism vs. antagonism) of a ligand. Thus, ligands which are able to bind to and/or alter the conformation of H12 are of particular importance when designing a ligand or assessing the binding of a ligand, for the estrogen receptor.

The present inventors have also found the reason why raloxifene has a different binding conformation to that of estradiol, the distinction lying in its active conformation but being unpredictable by virtue of it antagonistic action. The antagonism has been shown, by the present inventors, to be caused by a protruding portion on the raloxifene molecule which causes a large displacement of H12 relative to its conformation in the ER/estradiol complex.

Additionally, it has been found that at least the majority of such receptor proteins are in the form a dimer. Such dimerization leads to a potential route for disruption. Disruptions of this type can be used to predict antagonism or to produce antagonists. Disruptions may take the form of ligand binding which alters the conformation of the helices that comprise the dimerization interface or direct binding to the dimerization interface which then inhibits dimerization.

Further, the orientation of the ligand may be keyed to the receptor, in the dimeric or monomeric form. Furthermore, using the crystals of the present invention, the influence of ligand binding to the LDB on the receptor conformation can now be shown to have influences on the behavior of the receptor since it may disrupt the binding of co-activator, co-repressor, or heat-shock proteins. Previously, such predictions could not me made.

Production of Estrogen Receptor Crystals and their Application.

Preferably, the crystal is produced from a sequence comprising at least one hundered and fifty amino acids of the selected estrogen receptor. More preferably, the sequence comprises at least two hundred amino acids. Most preferably, the sequence comprises at least two hundred and fifty amino acids. Preferably, the sequence comprises at least a portion of the ligand binding domain of the estrogen receptor. More preferably, the sequence comprises the whole ligand binding domain of the estrogen receptor.

Typically ER LBDs are purified to homogeneity for crystallization. Purity of ER LBDs is measured with SDS-PAGE, mass spectrometry, and hydrophobic HPLC. The purified ER for crystallization should be at least 97.5% pure, preferably at least 99.0% pure, and most preferably at least 99.5% pure.

Preferably, the crystals used can withstand exposure to X-ray beams used to produce the diffraction pattern data necessary to solve the X-ray crystallographic structure. For example, crystals grown using estrogen receptor sequence bound to a various of ER ligands have been found to decompose during exposure to X-ray beams at room temperature, whereas crystals grown using estrogen receptor sequence bound to various ER ligands are freezable and are able to withstand exposure to X-ray beams.

Advantageously, the crystals have a resolution determined by X-ray crystallography of less than 3.5 Å and most preferably less than 2.8 Å. Preferably crystals grown using naturally occurring estradiol have an effective resolution of lower than 3.1 Å and crystals grown using raloxifene have an effective resolution of lower than 2.6 Å.

The production of such crystals has enabled the three dimensional structure of the ligand binding domain of the estrogen receptor to be mapped. Use of such crystals in conjunction with the map enables a better understanding of how estradiol and other estrogen bind to the estrogen receptor with precision. This technique can also enable the design of estrogen antagonists since the binding site is known.

For example, in the prior art it has been proposed (see Grease et al., J. Med. Chem. (1997), 40:146-147) to prepare raloxifene analogues using a number of substitutions to the 2-aryl group, one of which is 2-napthyl and shows efficacy in preventing bone loss at the expense of a loss of binding affinity using, for example a 4′-OH substituent (resulting in a slight affinity loss compared to just a napthyl). Having mapped the estrogen receptor, upon reviewing Formula X below, the fit of such a compound into the estrogen binding site comes intuitively apparent, that is, an amalgamation of the D-ring of estradiol and the pendant position-2 aryl substituent, but using the map, the present inventors have found that a 6′-OH, or even a 5′-OH will be more favorable for affinity.

For example, use of such methods has allowed the present inventors to determine the different binding modes of different steroid hormones to the estrogen receptor such as how the binding of testosterone to the estrogen receptor, which is imperfect binding, differs from that of estradiol. In particular, such models show that there is (1) electrostatic repulsion between the C-3 carbonyl oxygen atom of testosterone and the carboxylate of Glu-353 and (2) steric repulsion between the side chain of the C-18 methyl group of testosterone and the side chain of Leu-387 which accounts for the much lower affinity of testosterone compared to estradiol for the estrogen receptor. The steric hindrance and other stereochemical features of molecules has been found to affect the flexibility, that is the ability to alter the tertiary structure, of the ligand binding domain which therefore affects the perturbility of the ligand binding domain. Therefore, using the crystals of the present invention it is now possible for it to be clearly seen how estradiol binds to the estrogen receptor and hence the structural reasons why a compound behaves as an estrogen can not only be understood but also predicted. This enables an understanding of the promiscuity of the estrogen receptor—its ability to bind a variety of structurally diverse ligands. This understanding can be applied to a greater or lesser extent to all steroid hormone receptors, especially the glucocorticoid receptor.

Crystals of the estrogen receptor binding domain can be used as models in methods for the design of synthetic compounds intended to bind to the receptor. Such models show why very slight difference in chemical moieties of a ligand potentially have widely varying binding affinities. Hence, the three dimensional structure of the ligand binding domain can be used a pharmaceutical model for compounds which bind to estrogen receptors.

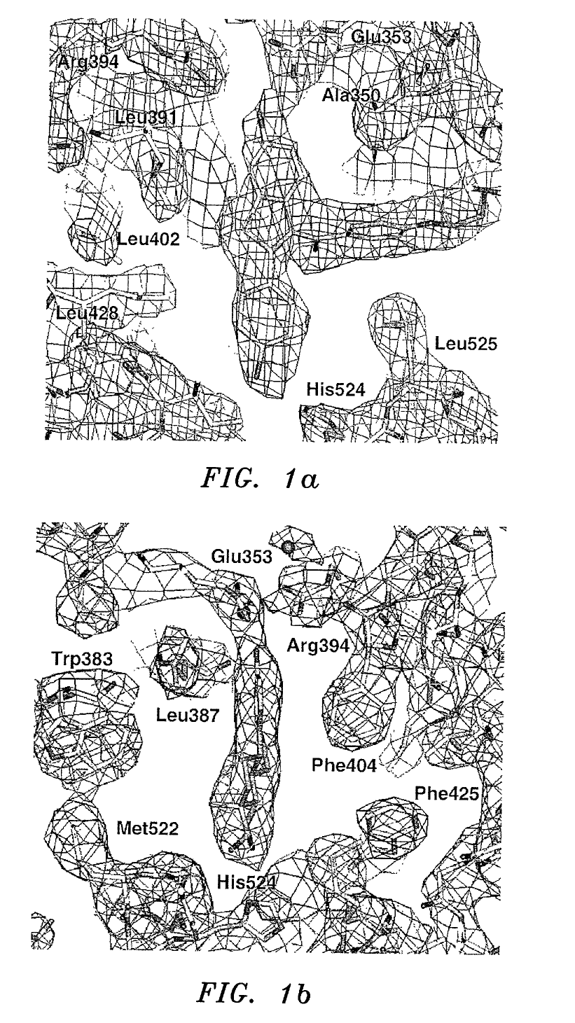

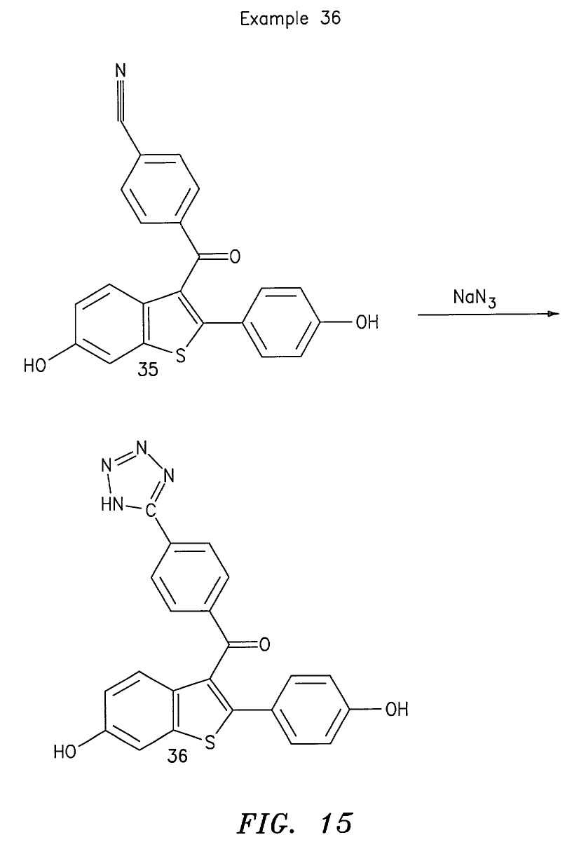

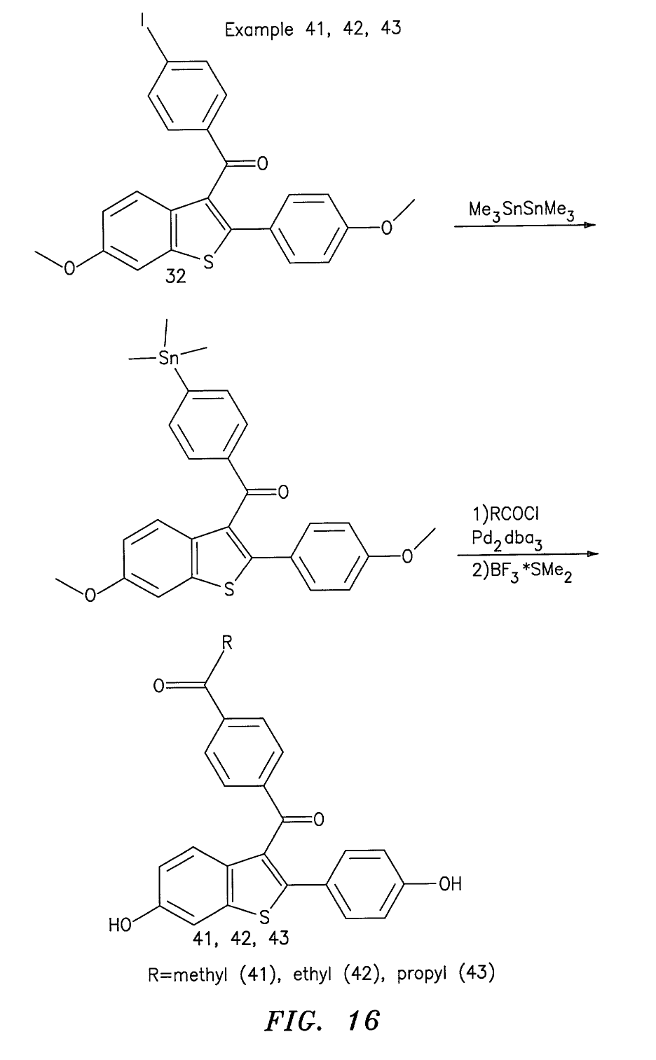

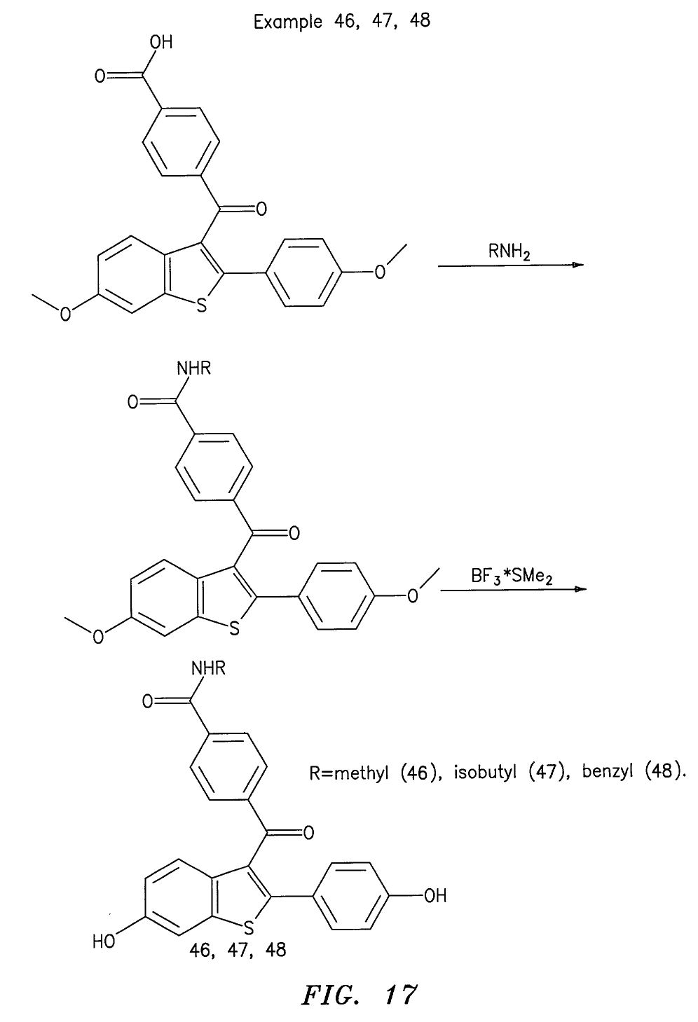

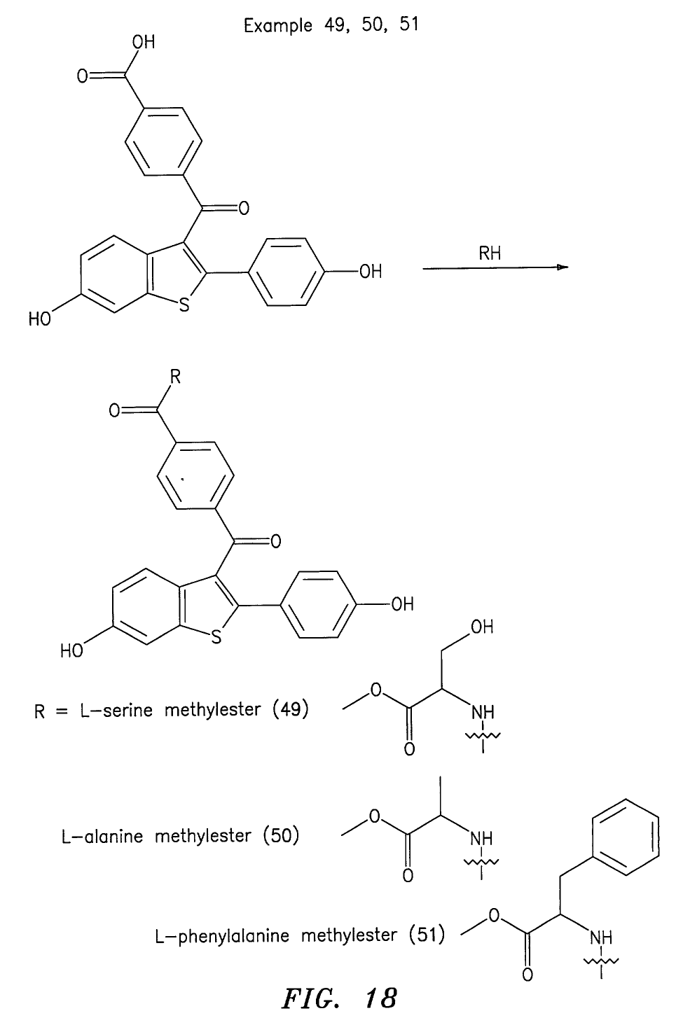

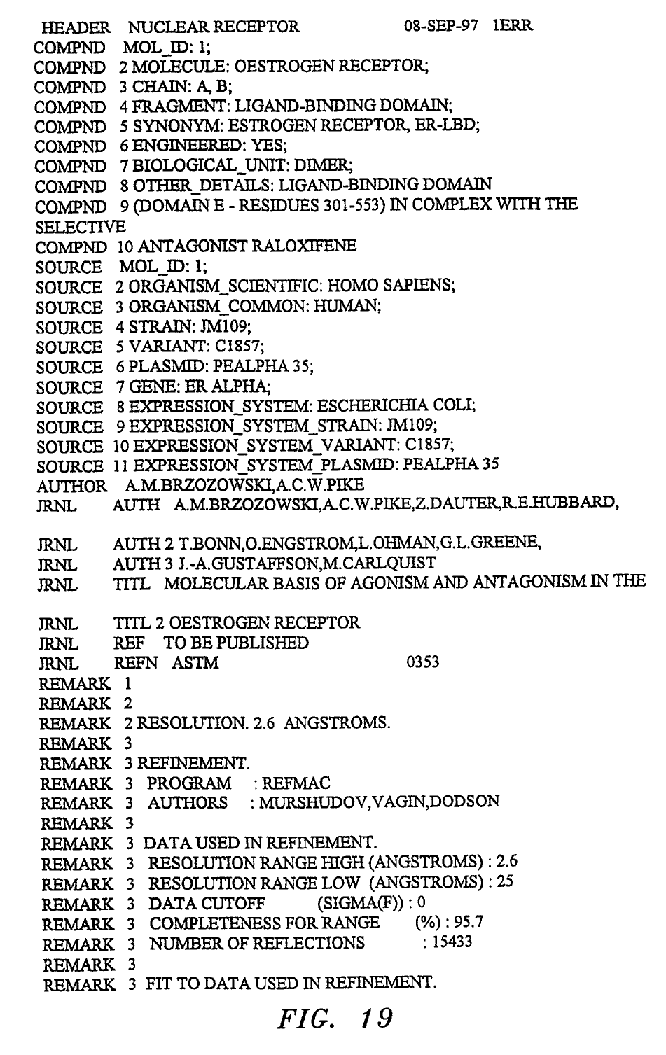

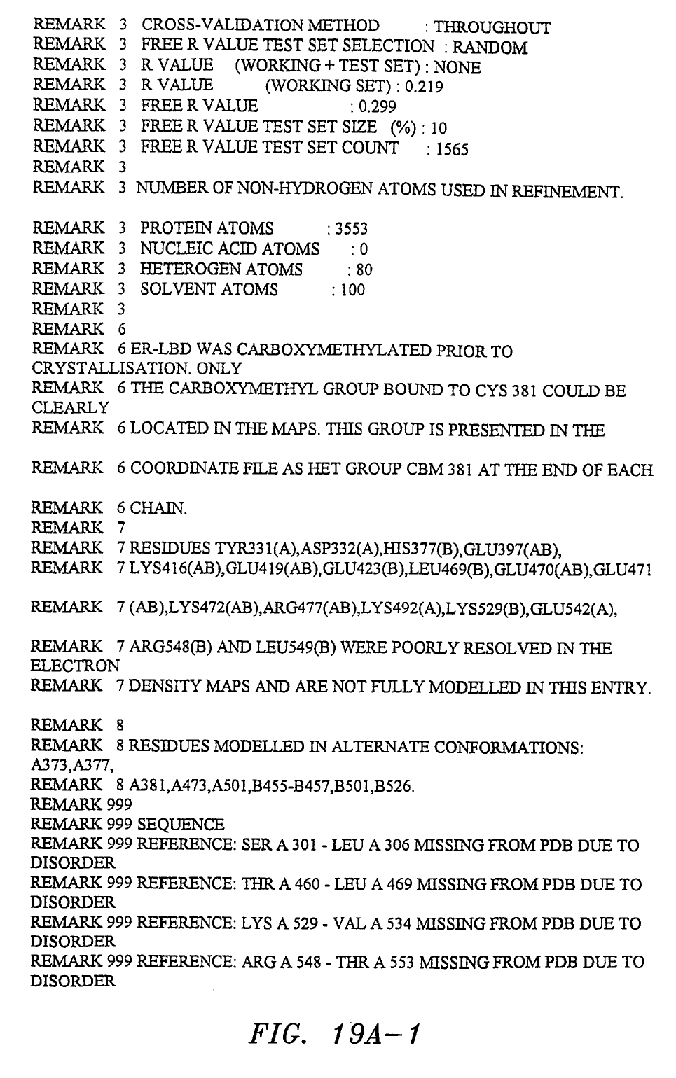

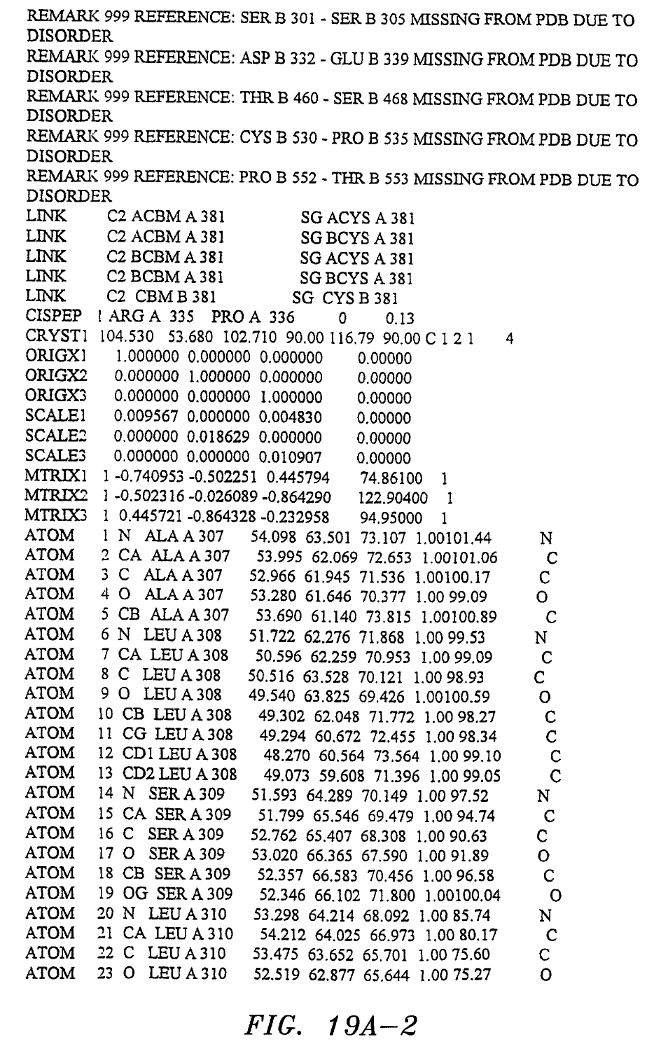

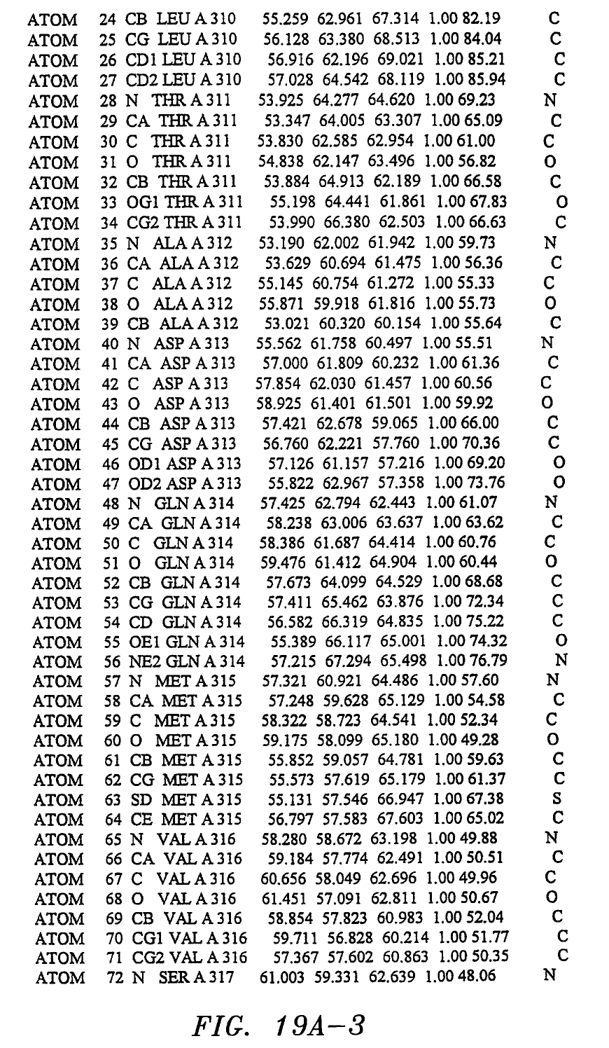

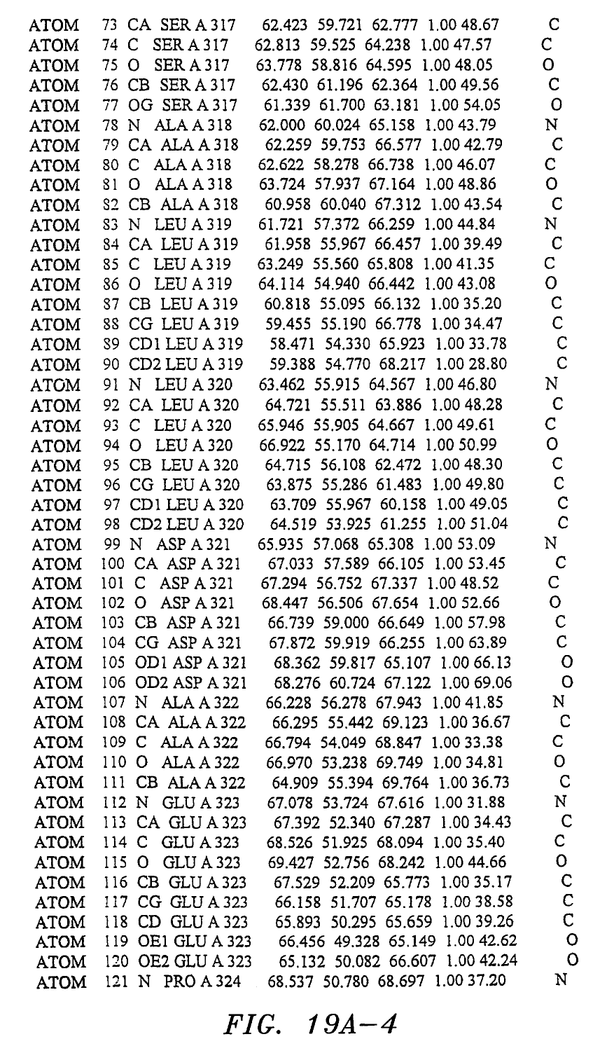

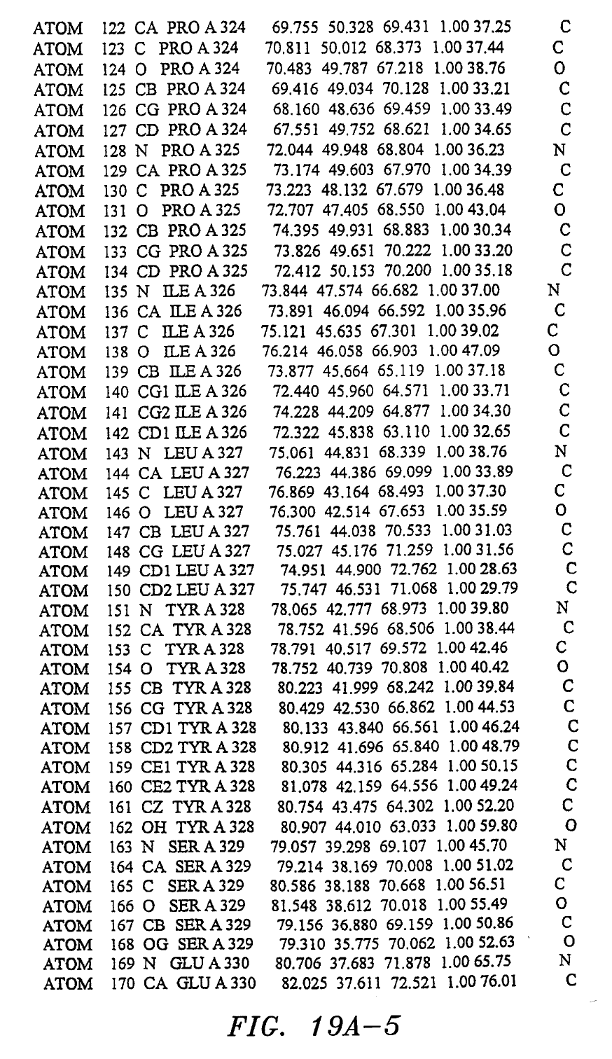

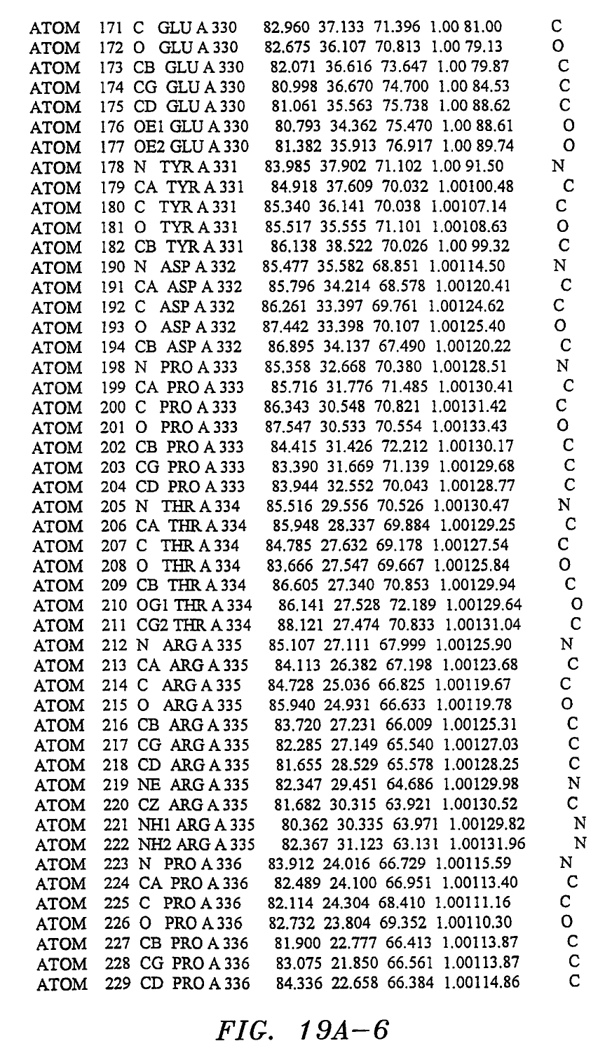

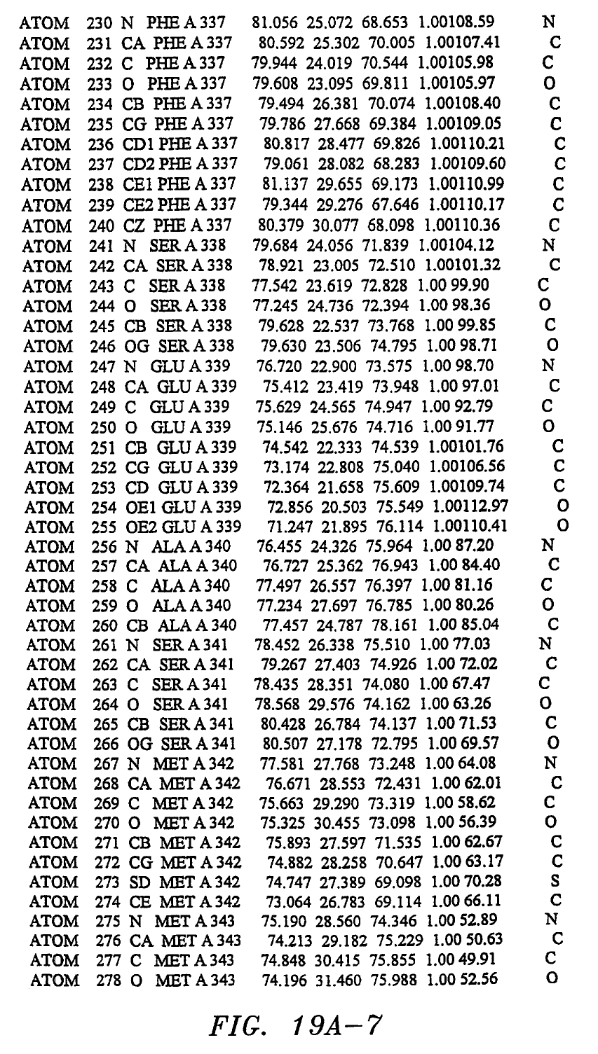

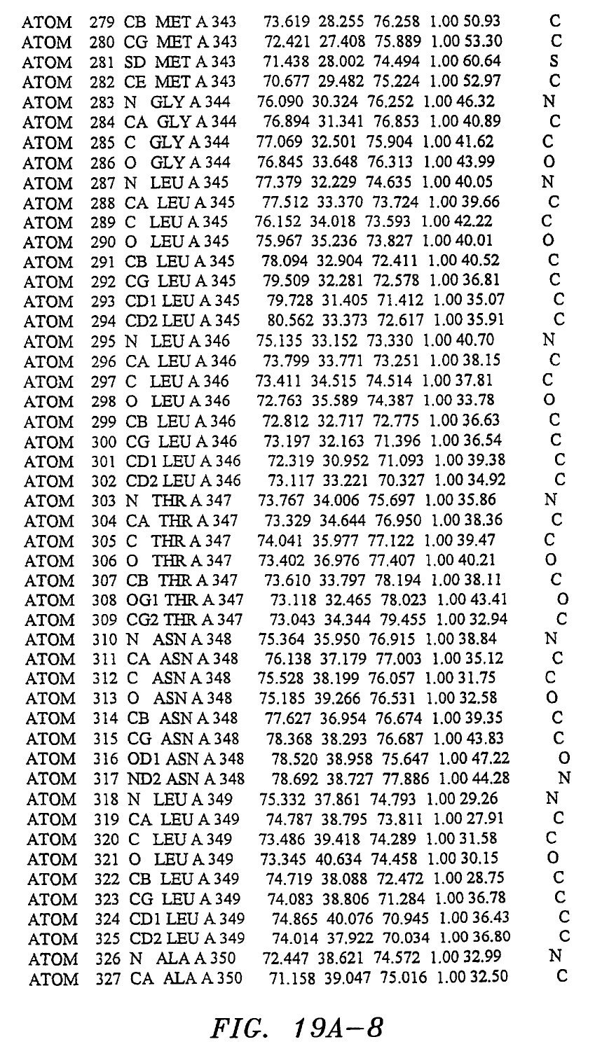

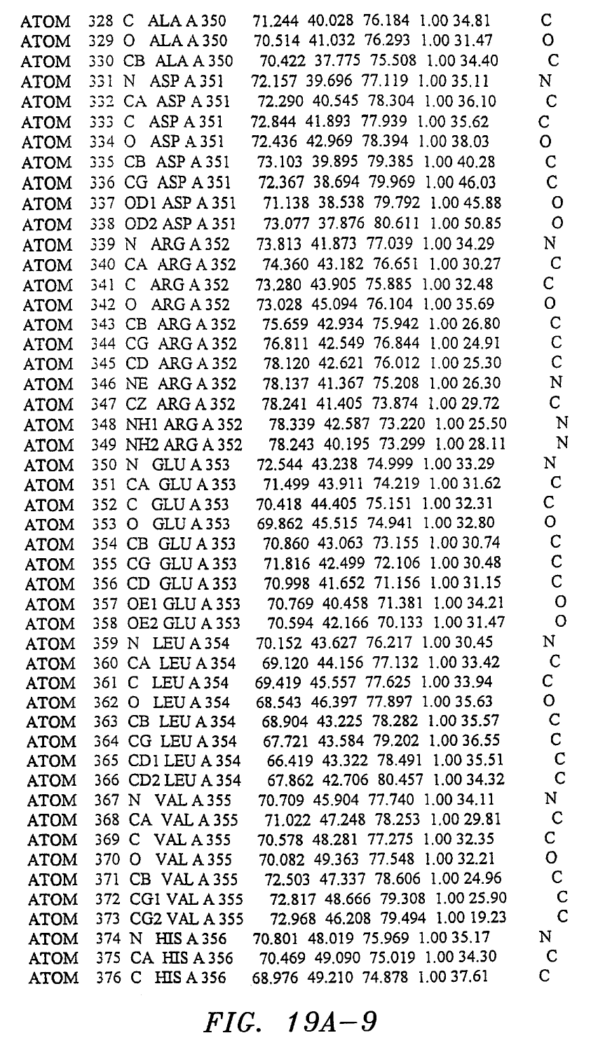

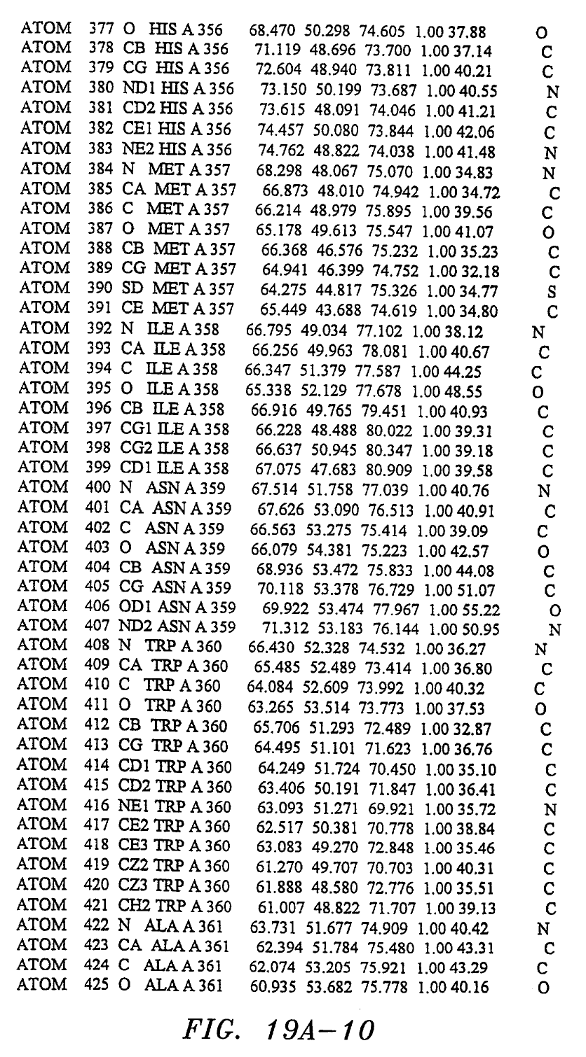

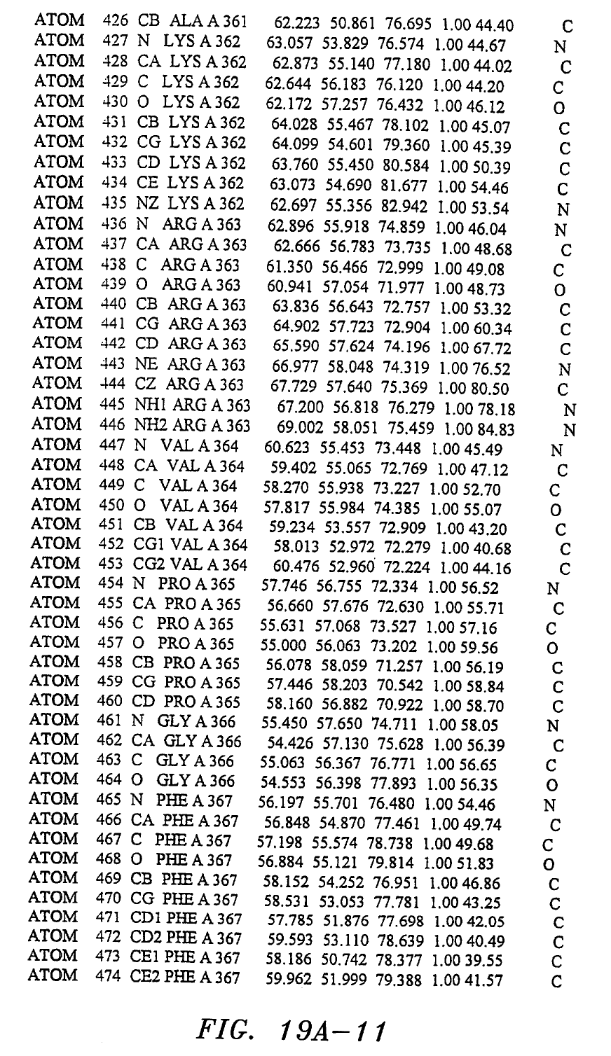

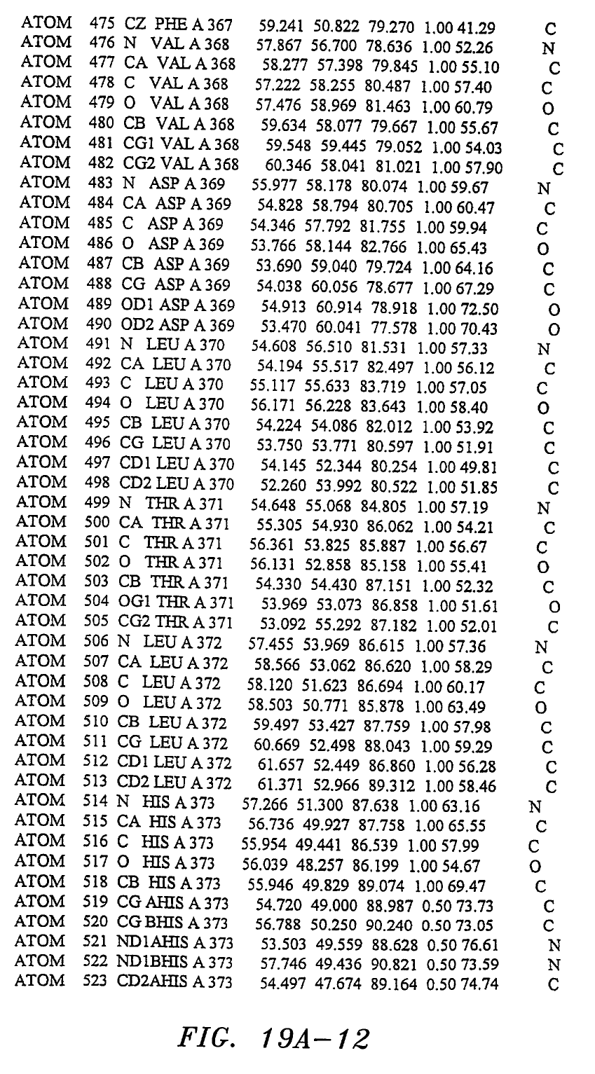

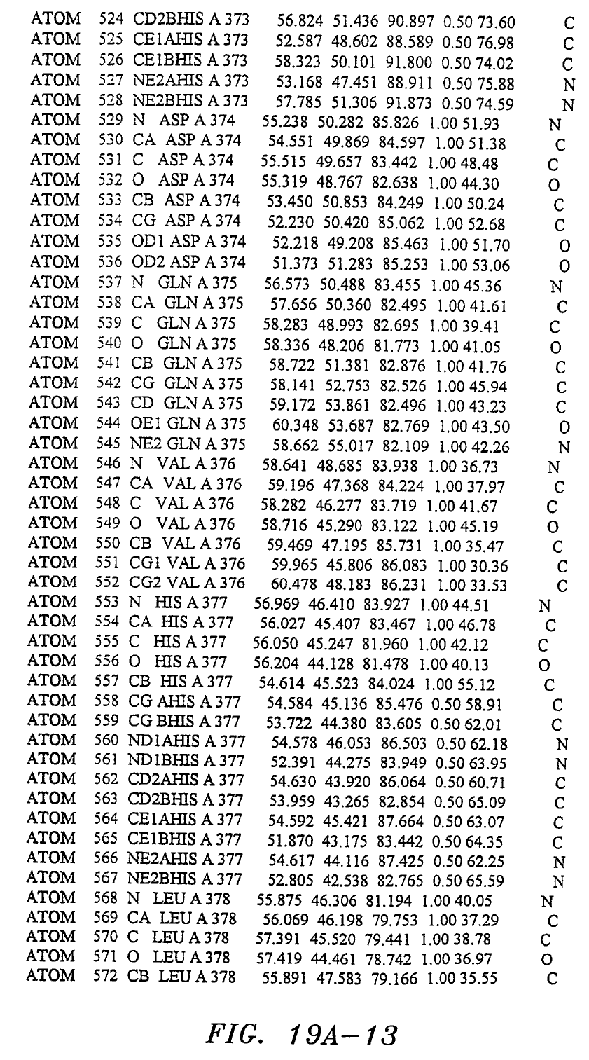

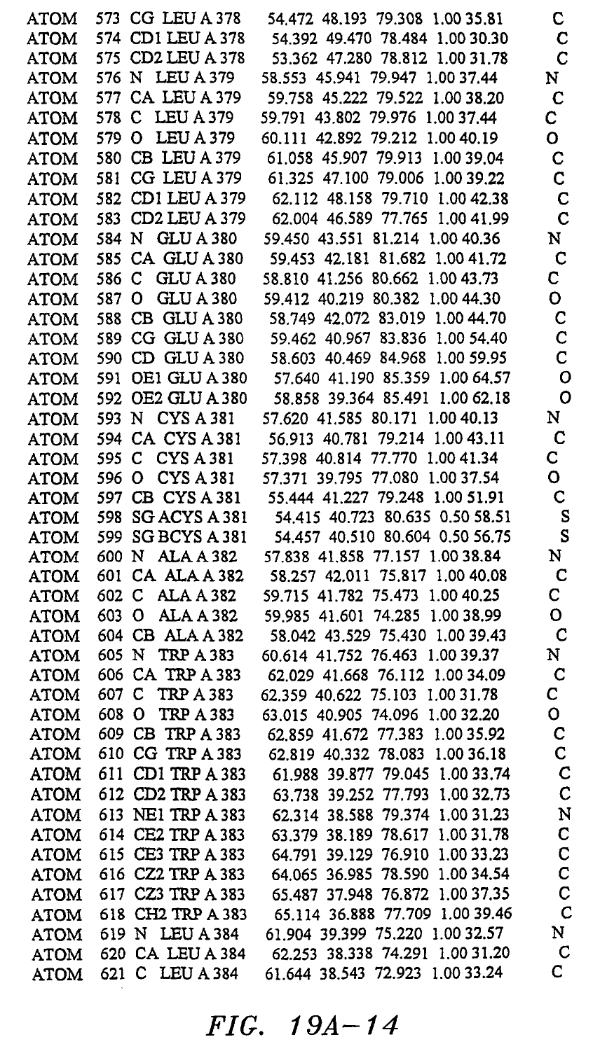

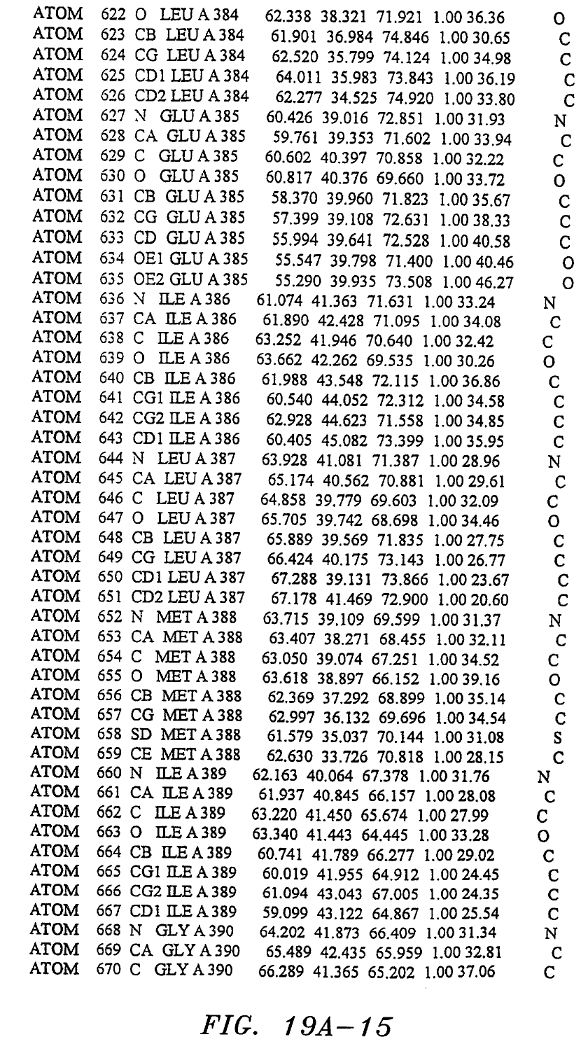

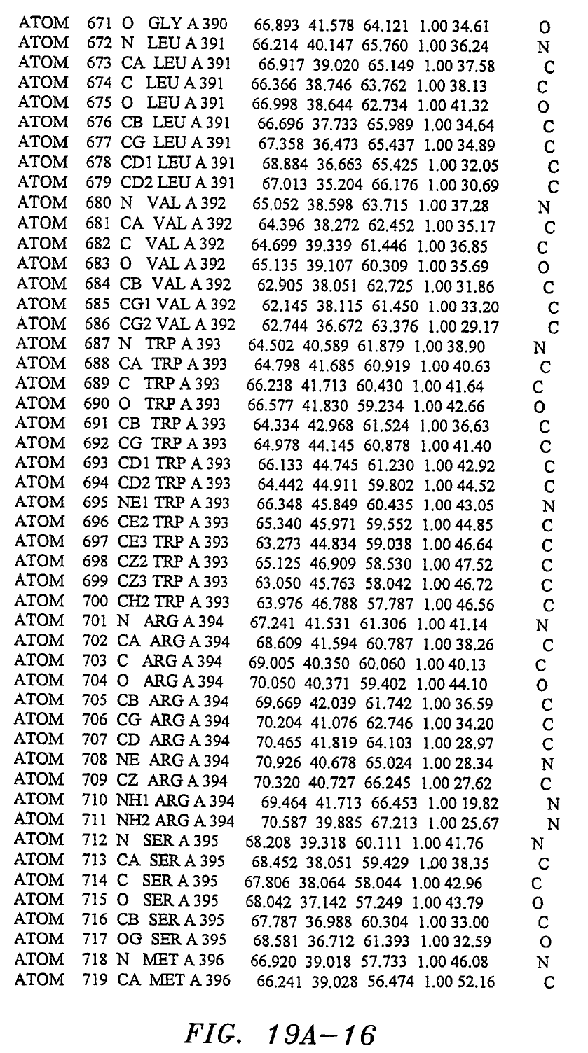

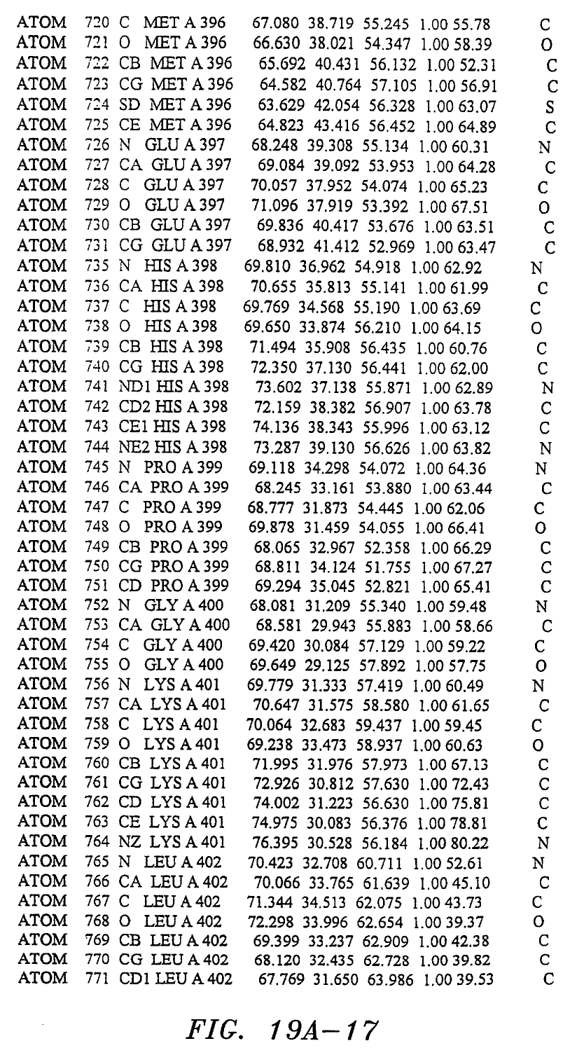

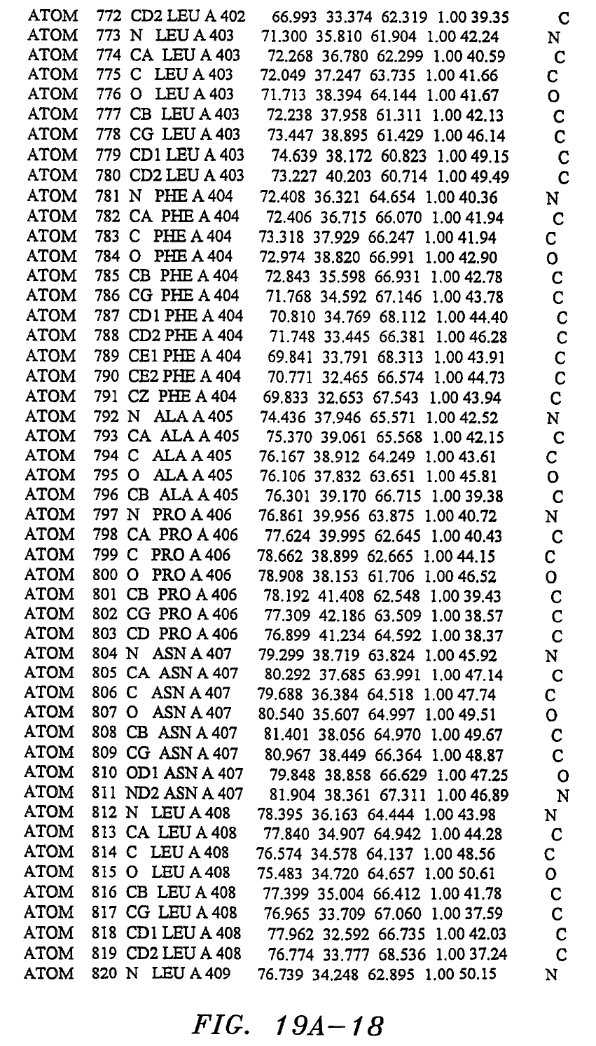

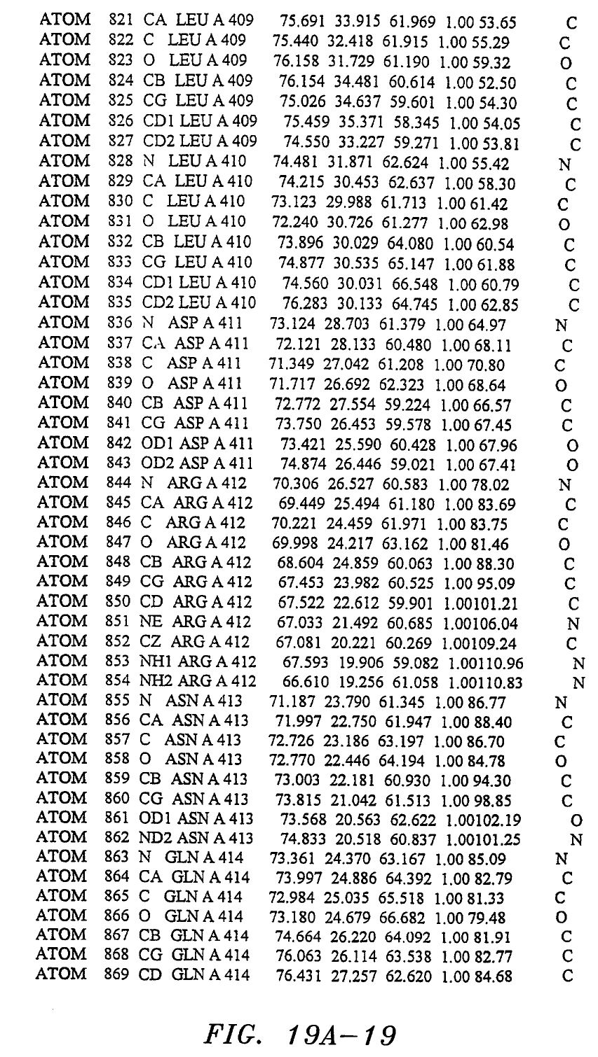

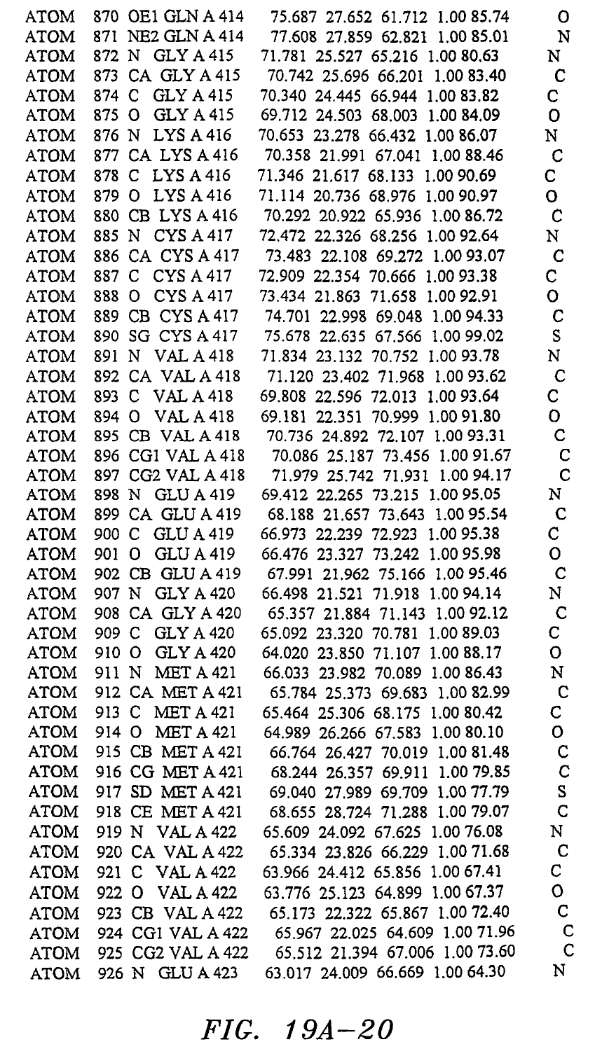

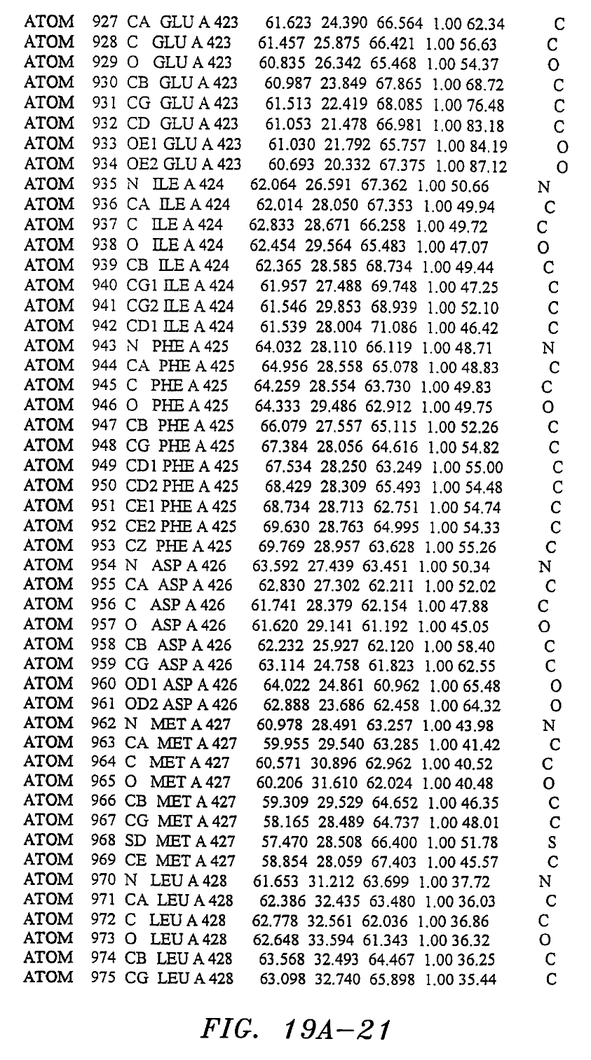

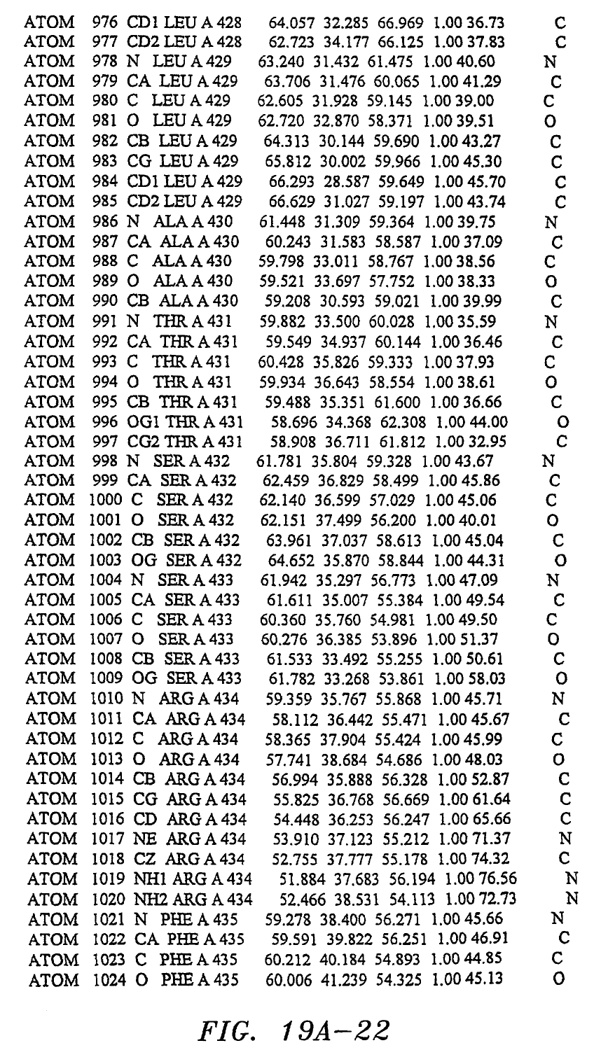

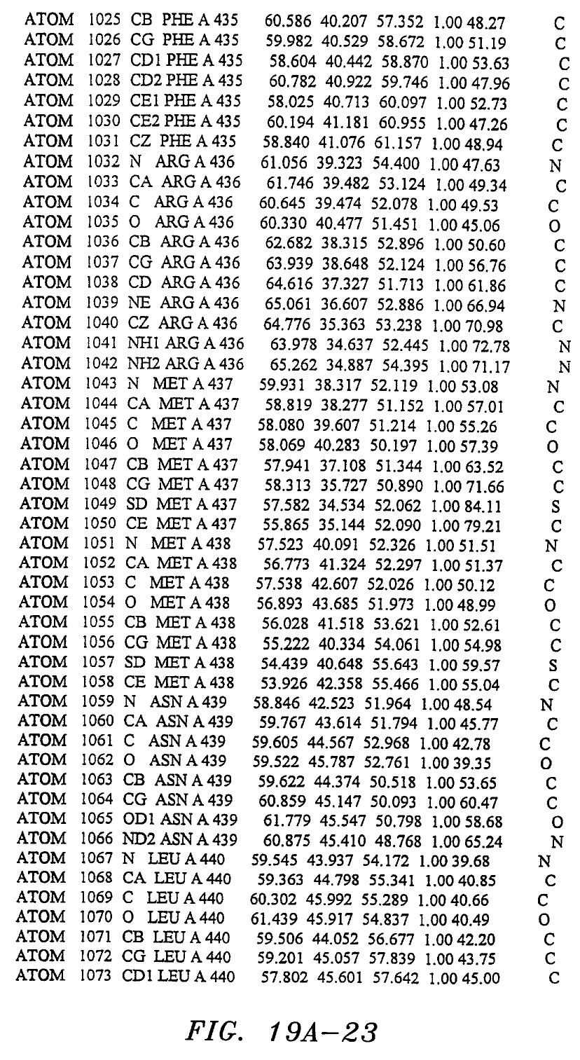

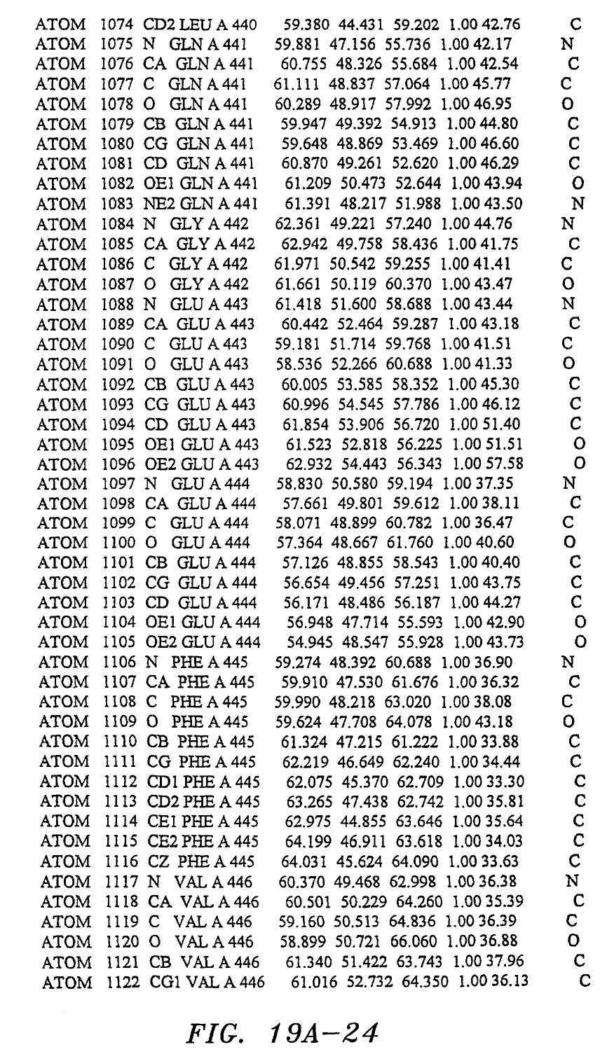

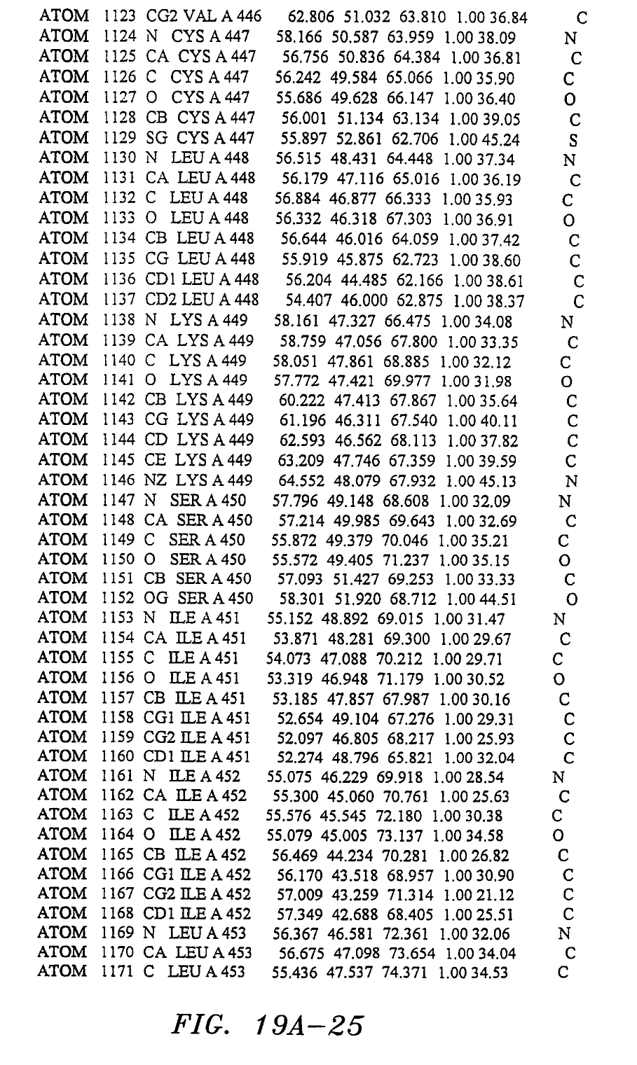

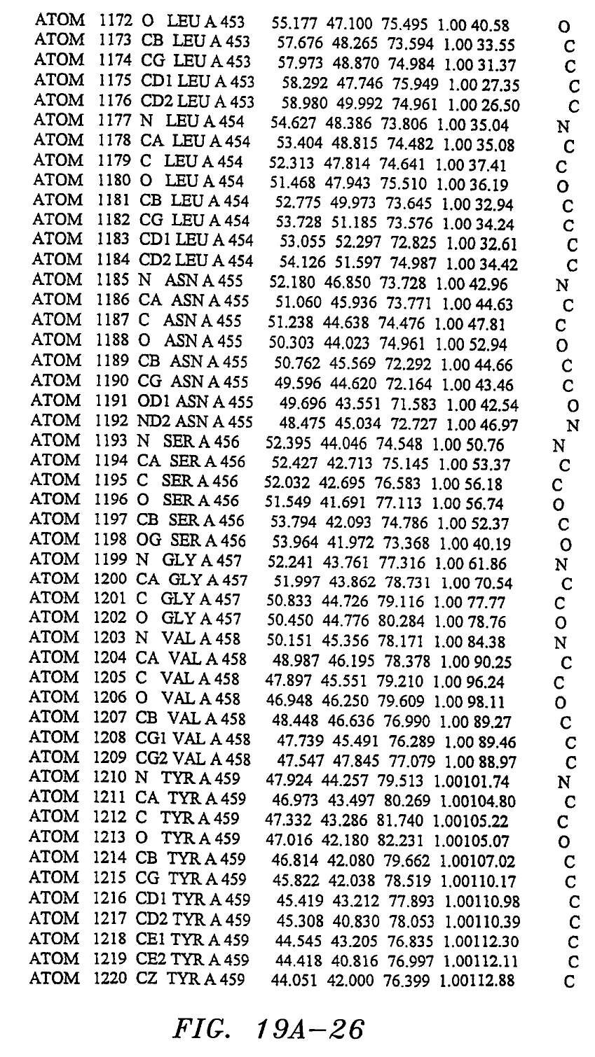

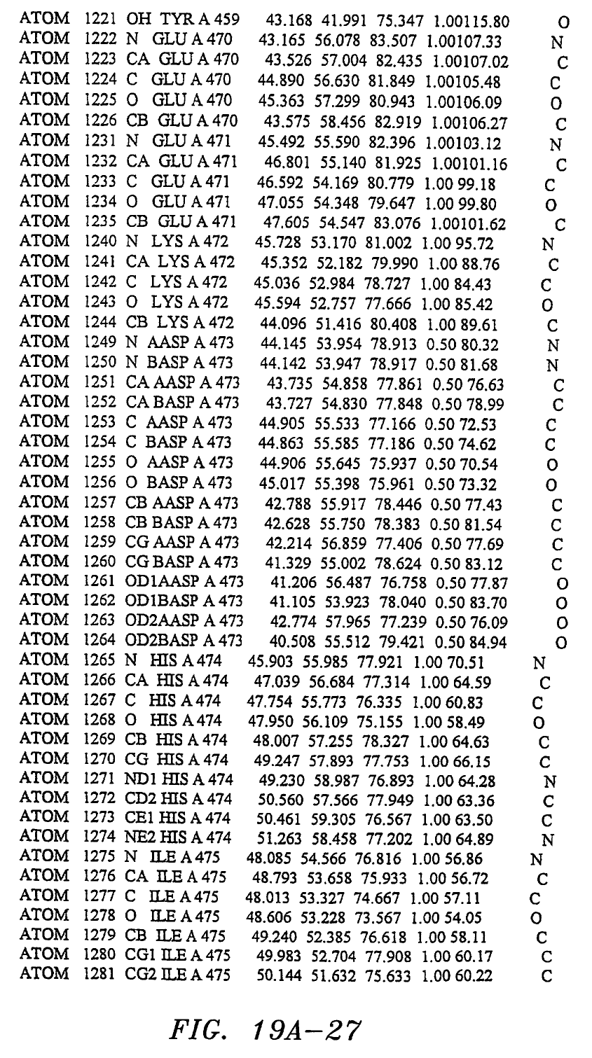

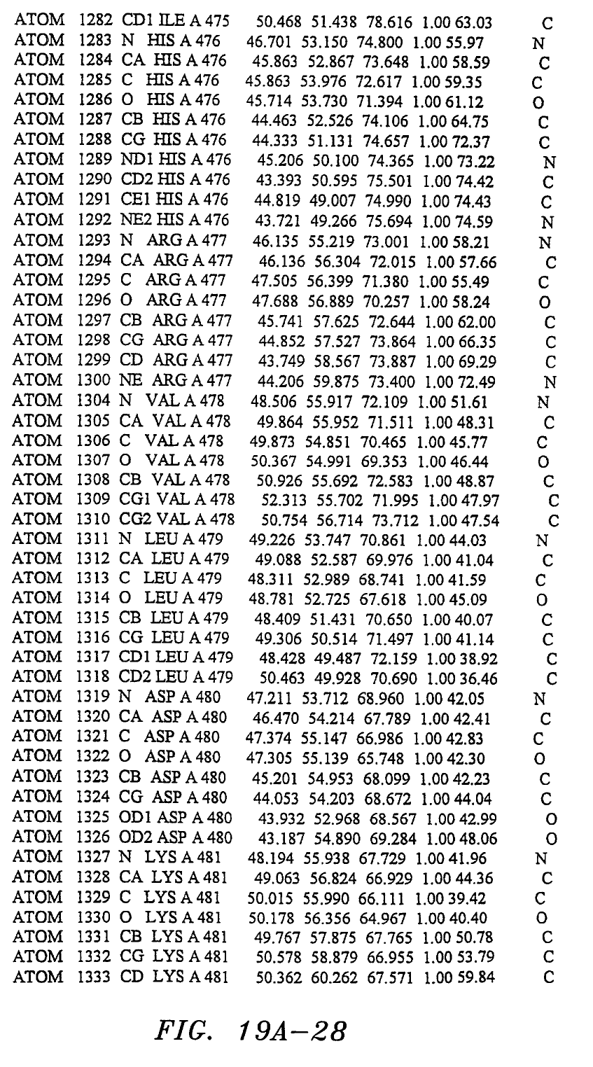

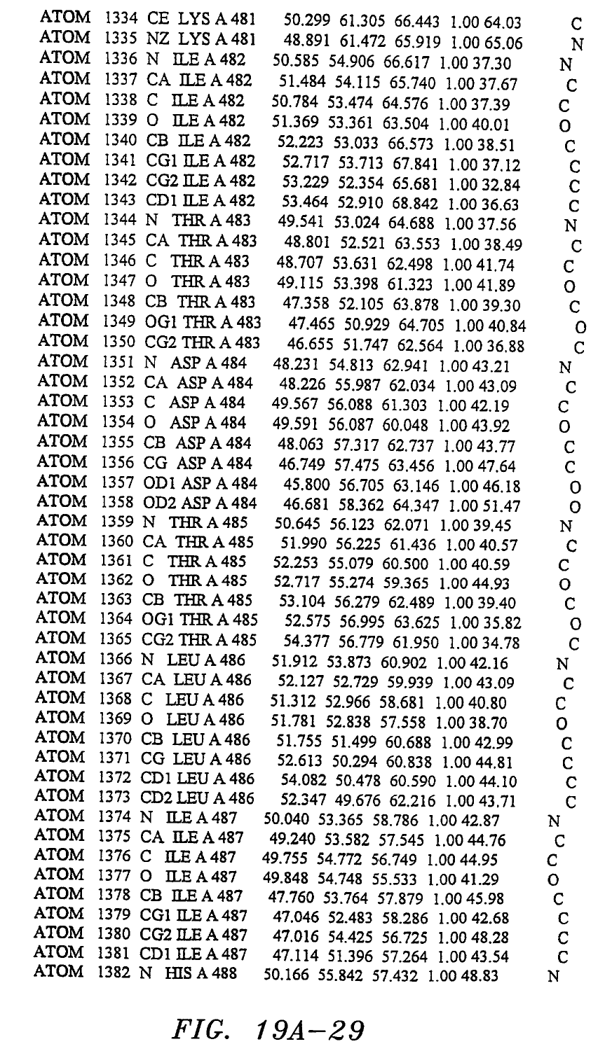

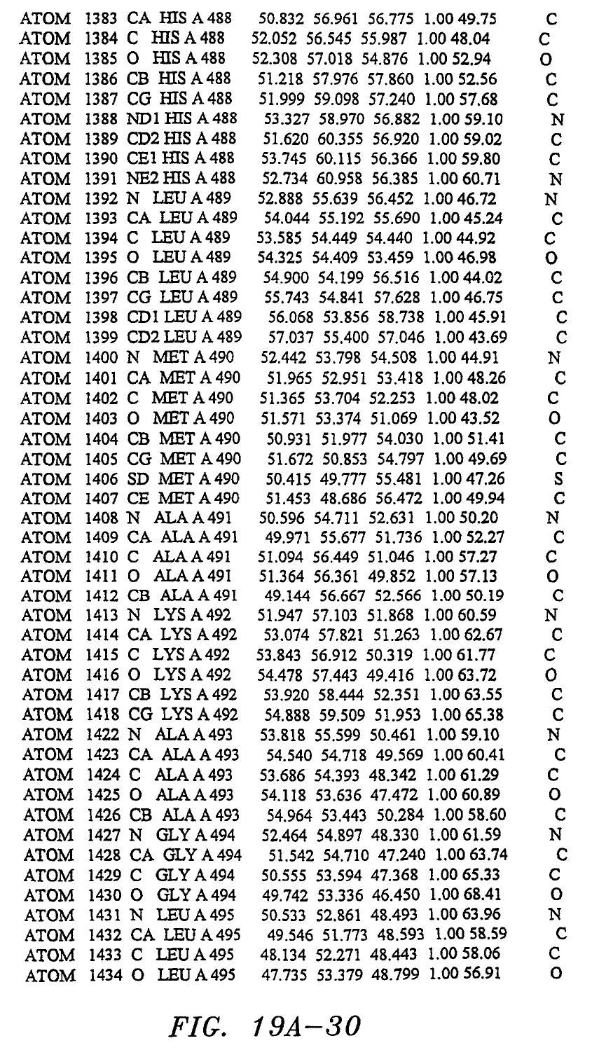

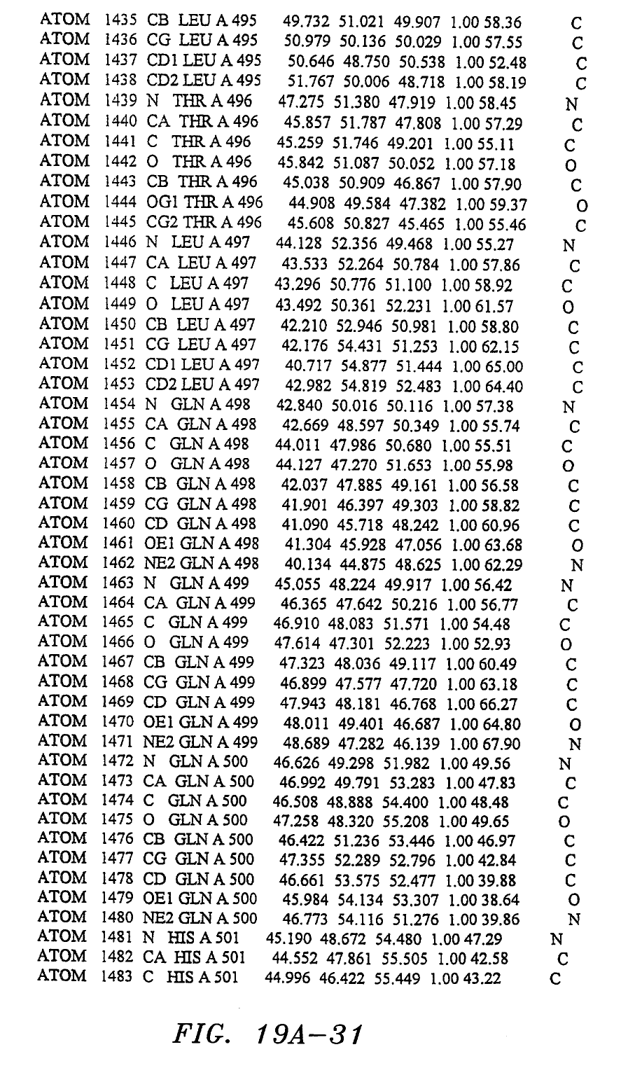

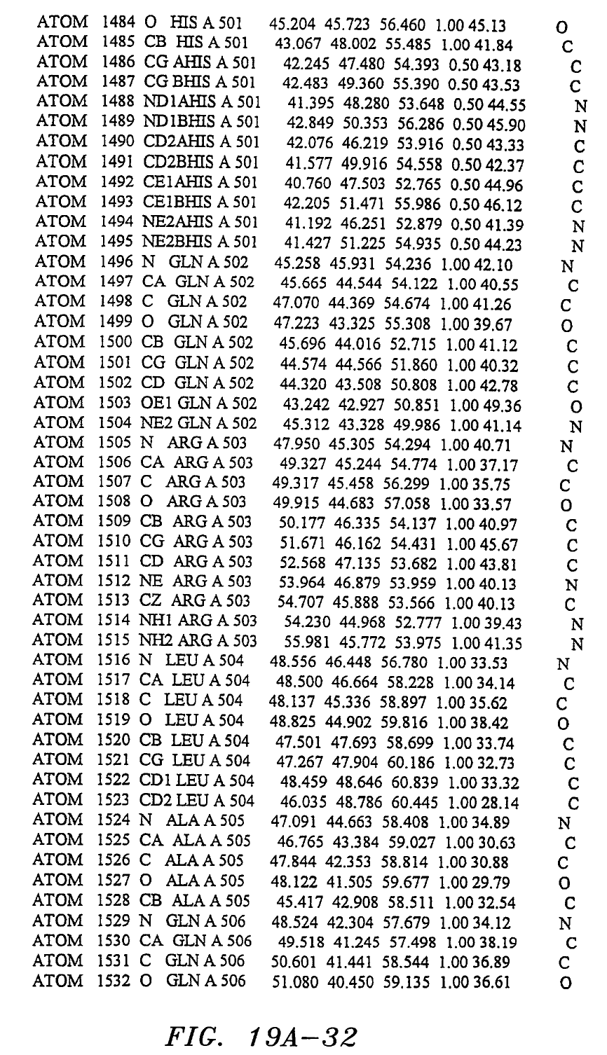

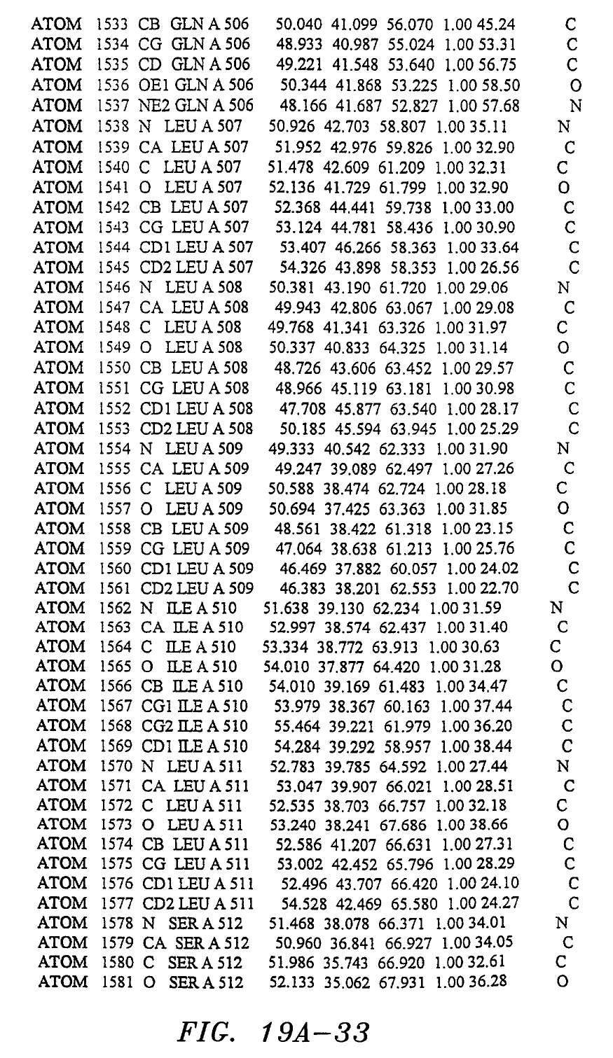

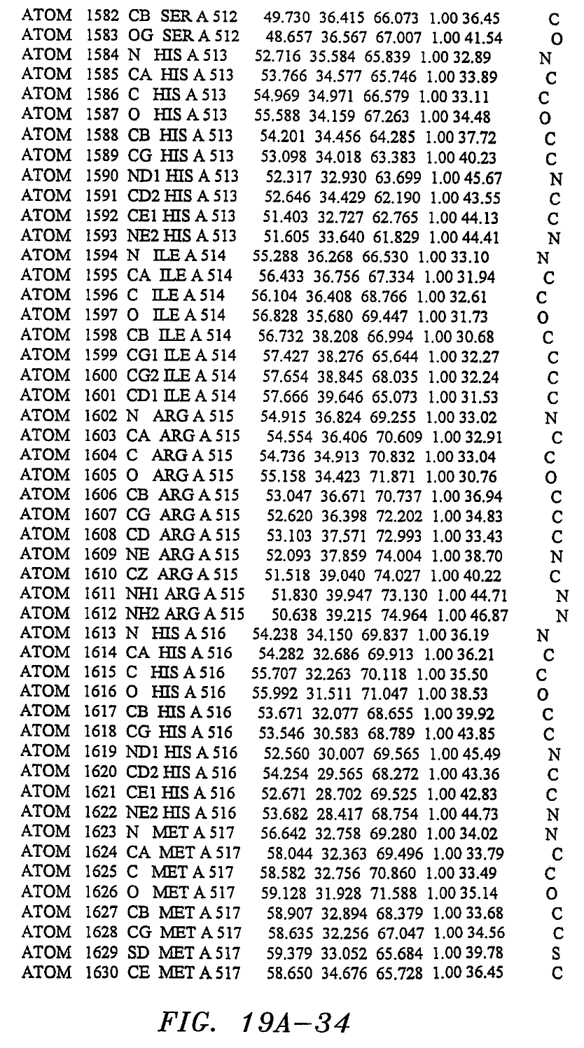

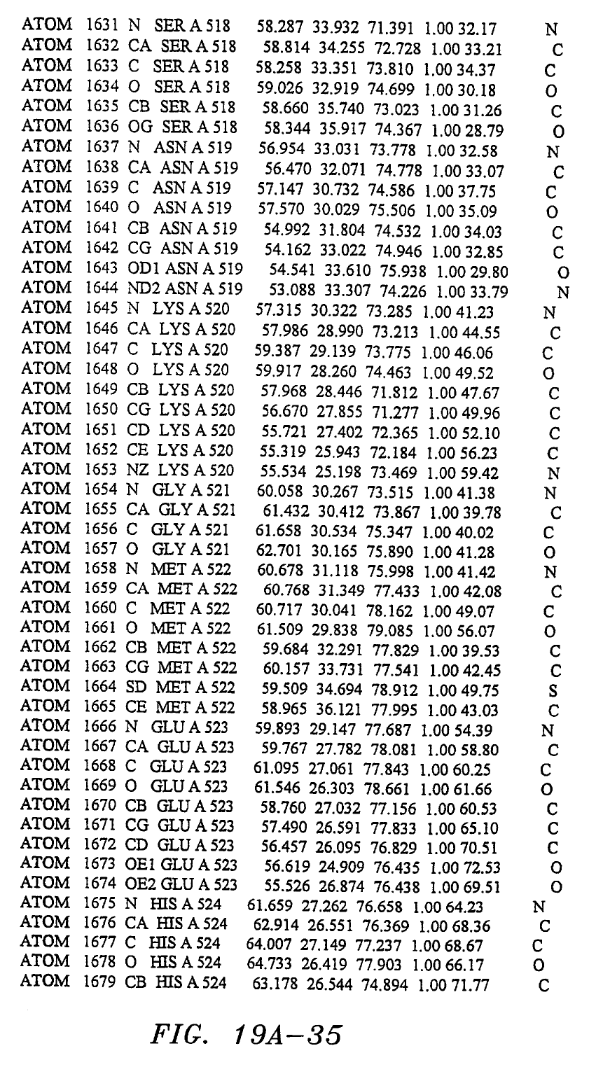

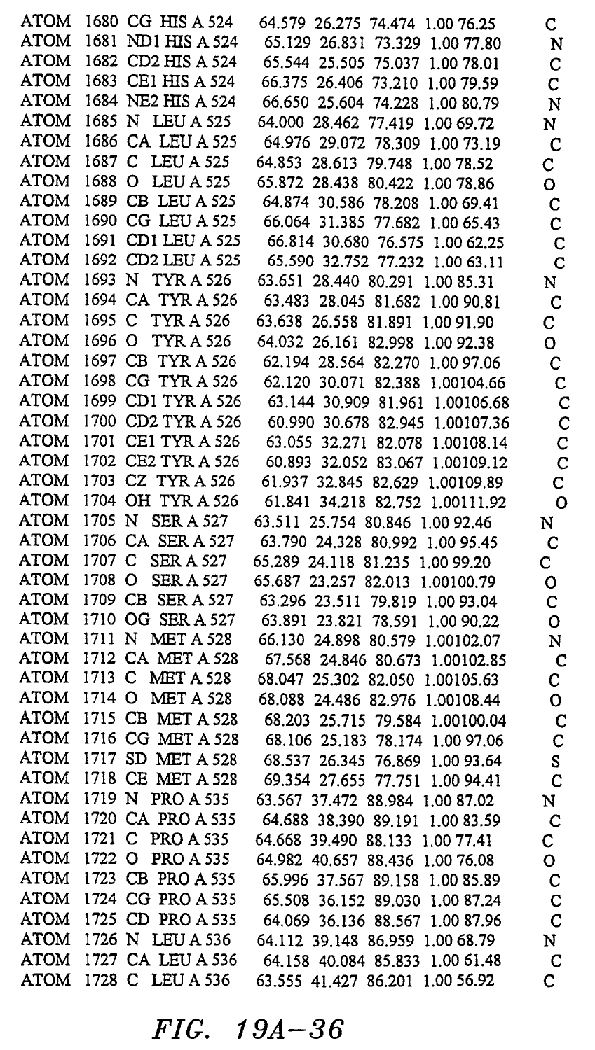

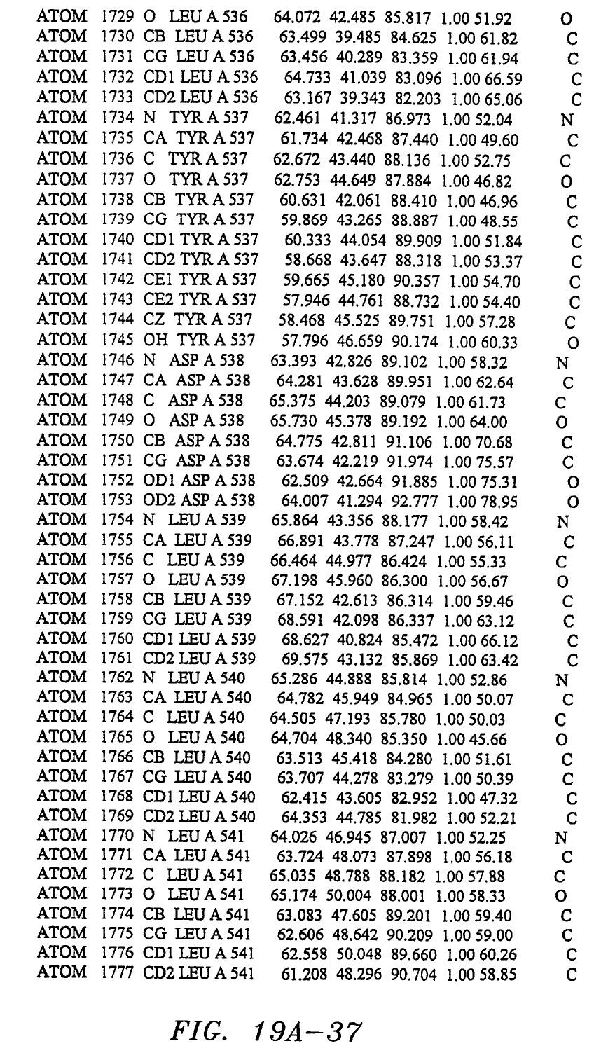

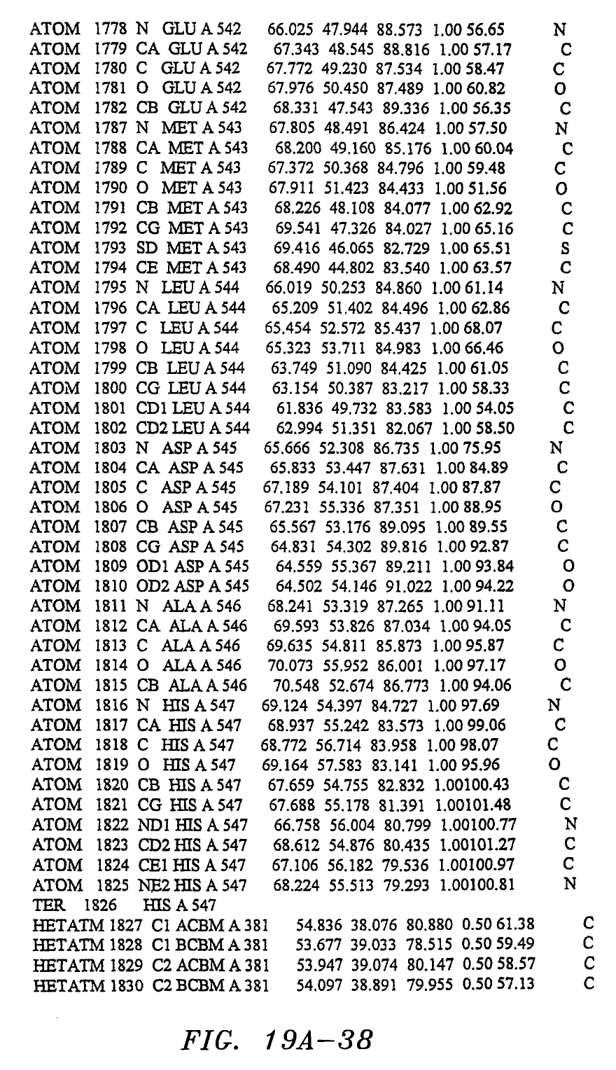

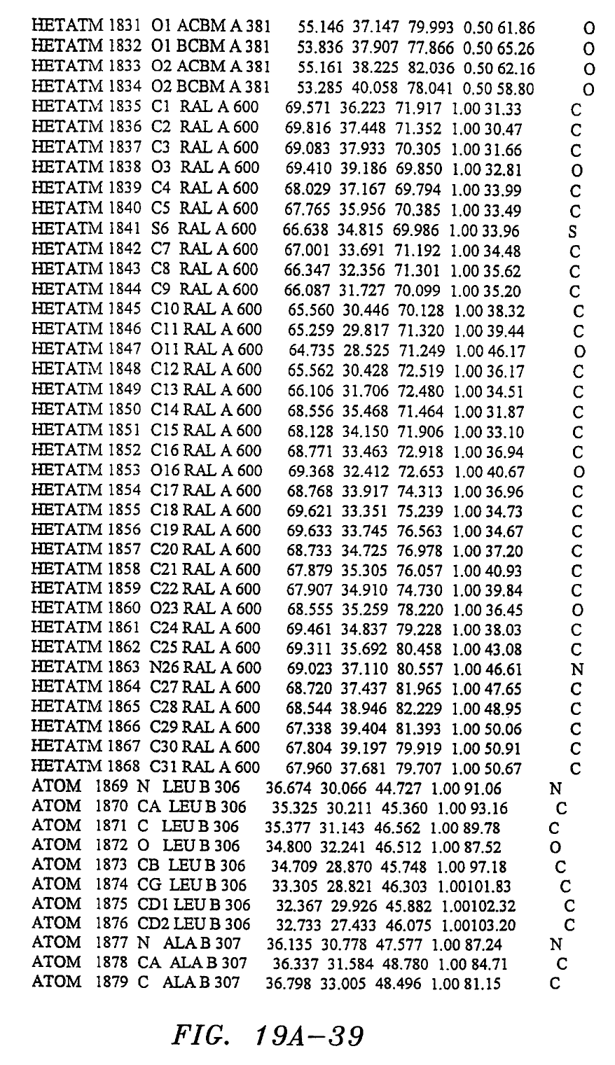

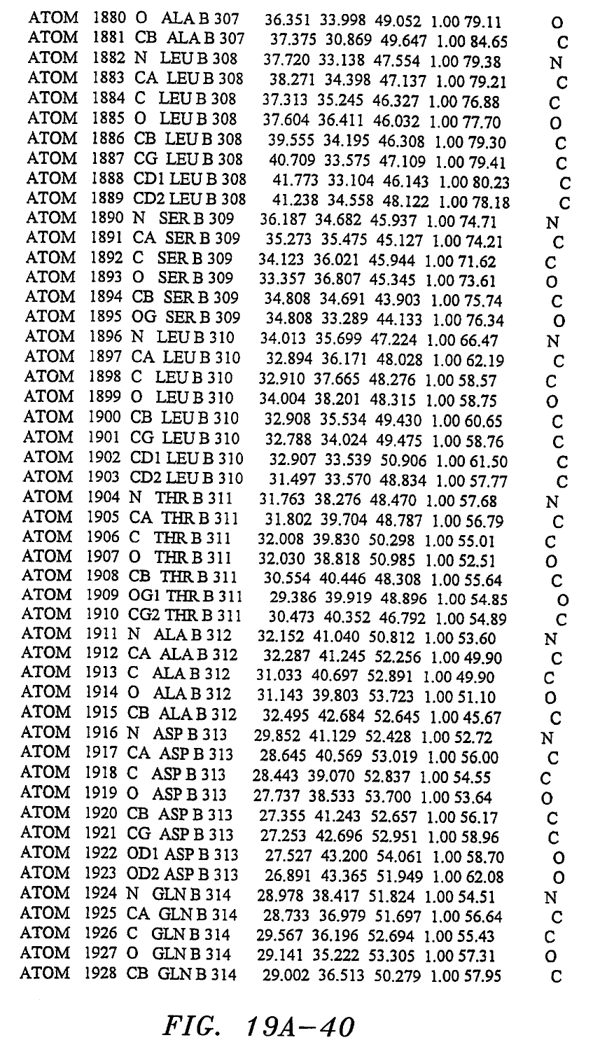

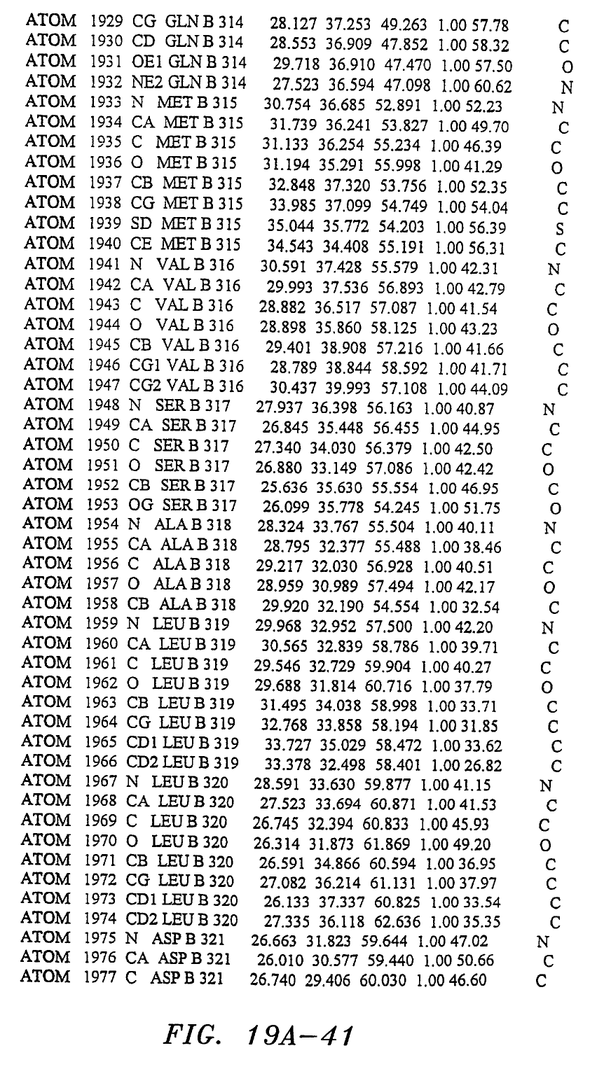

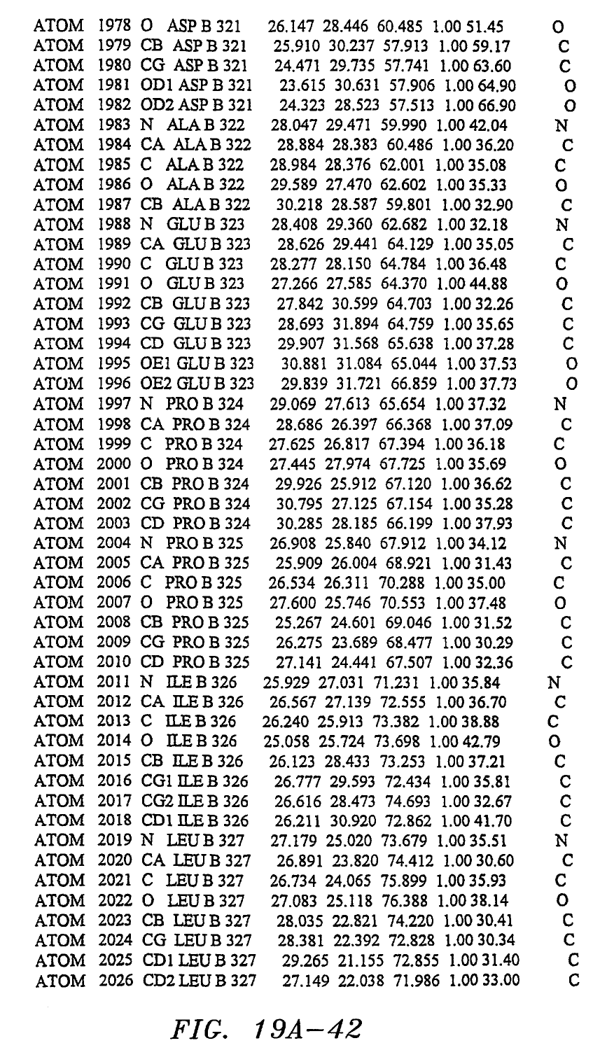

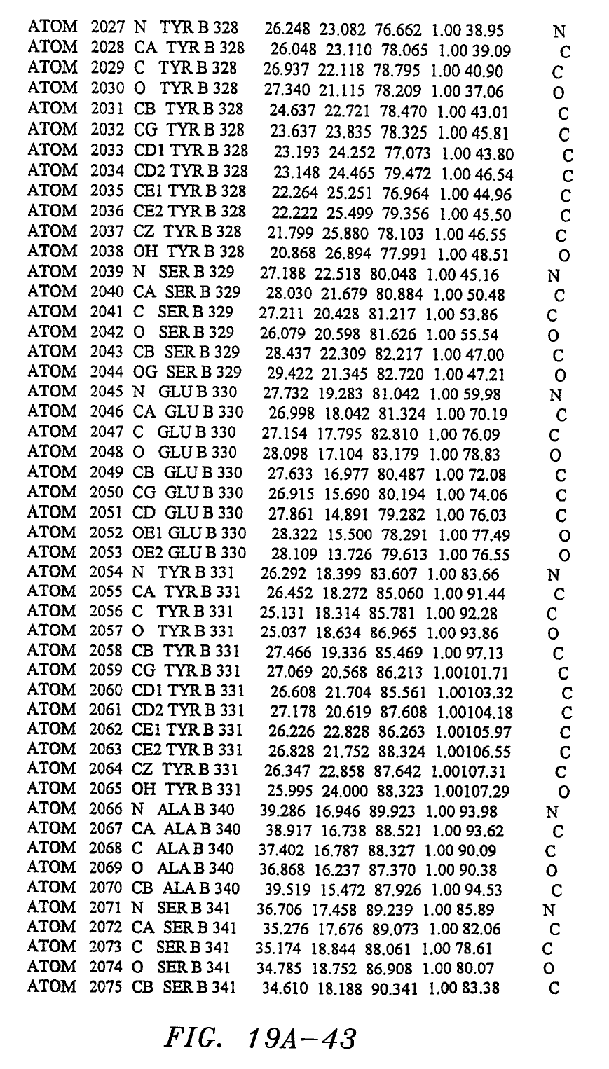

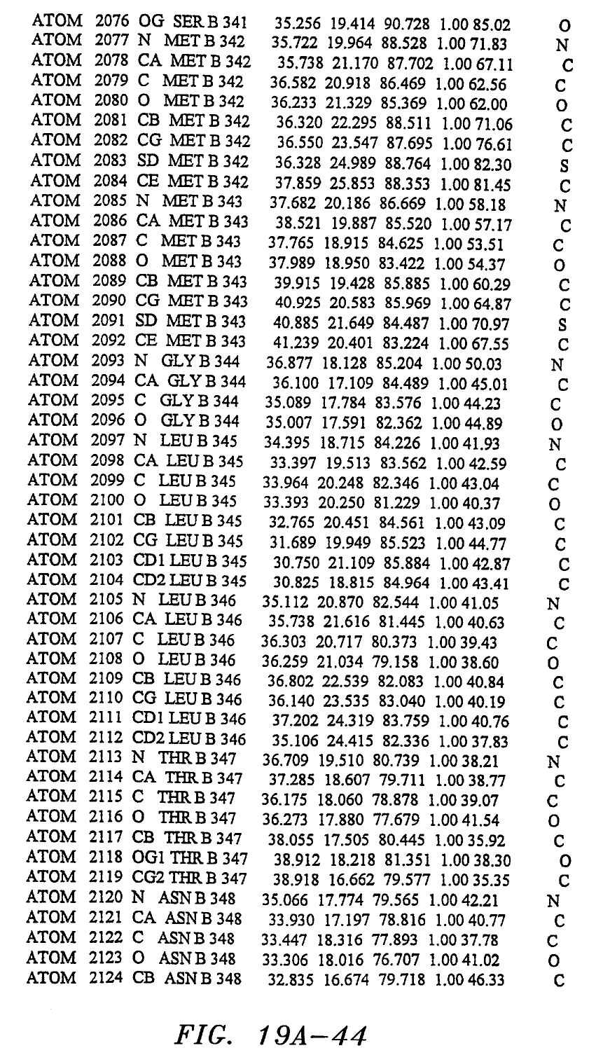

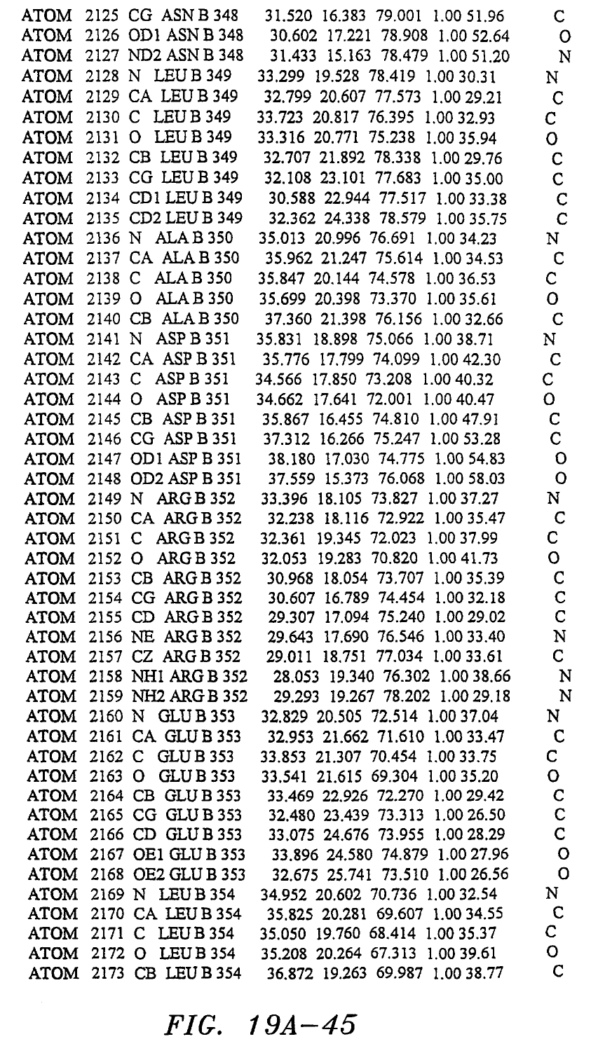

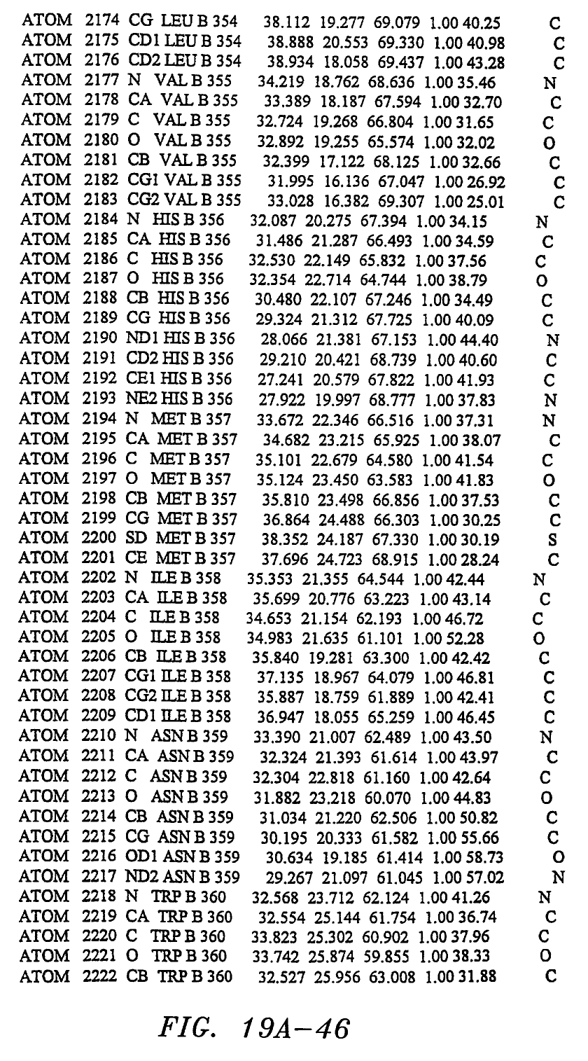

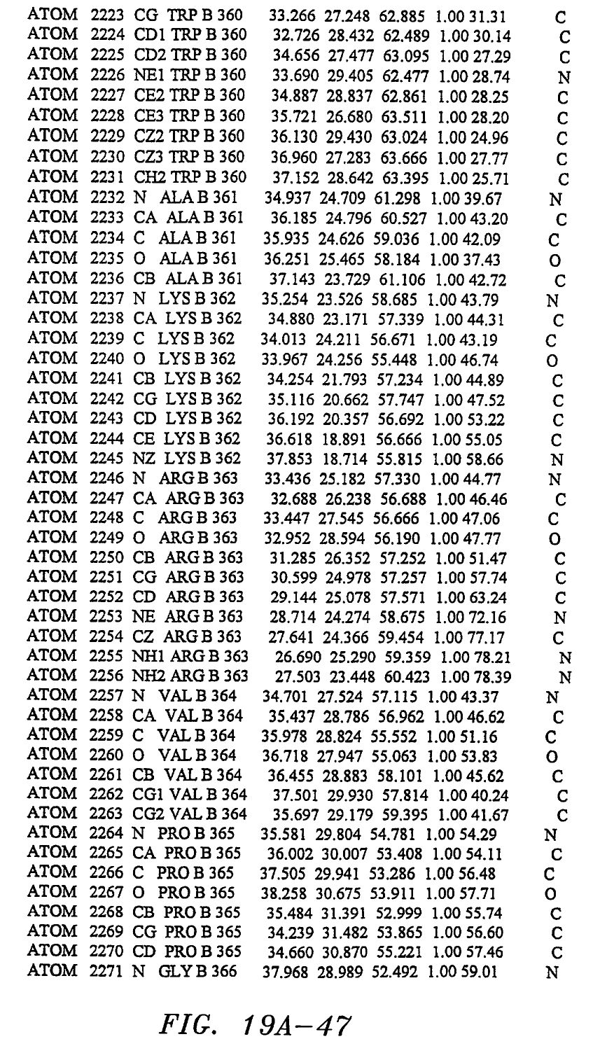

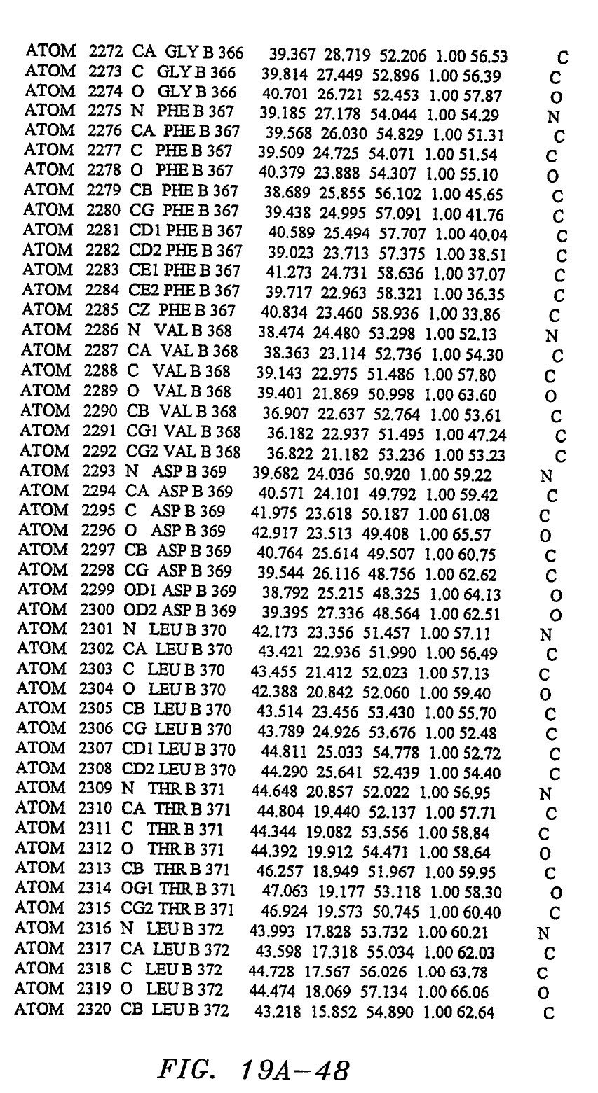

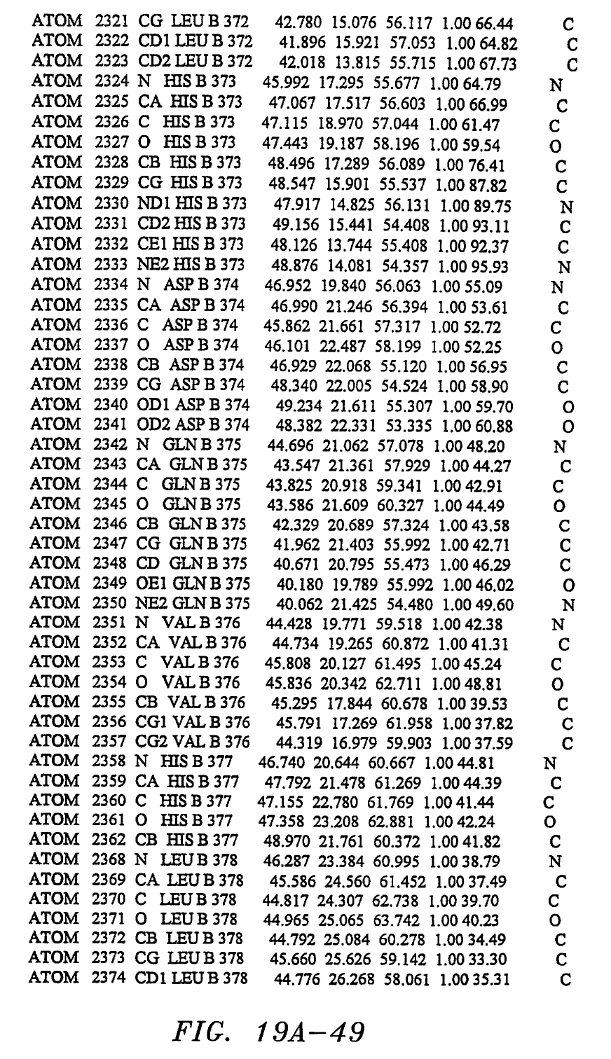

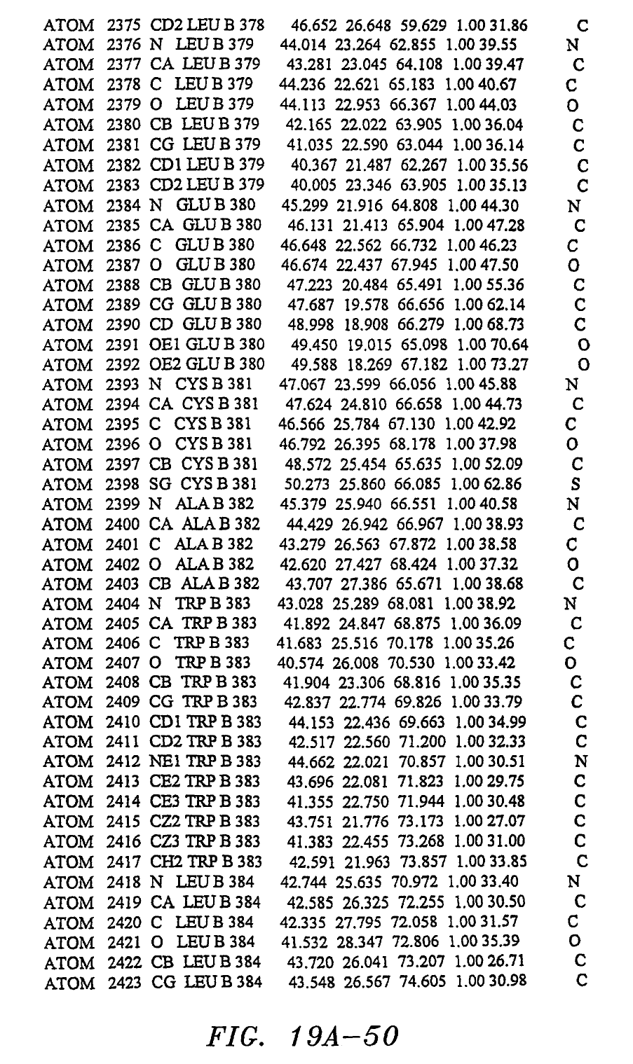

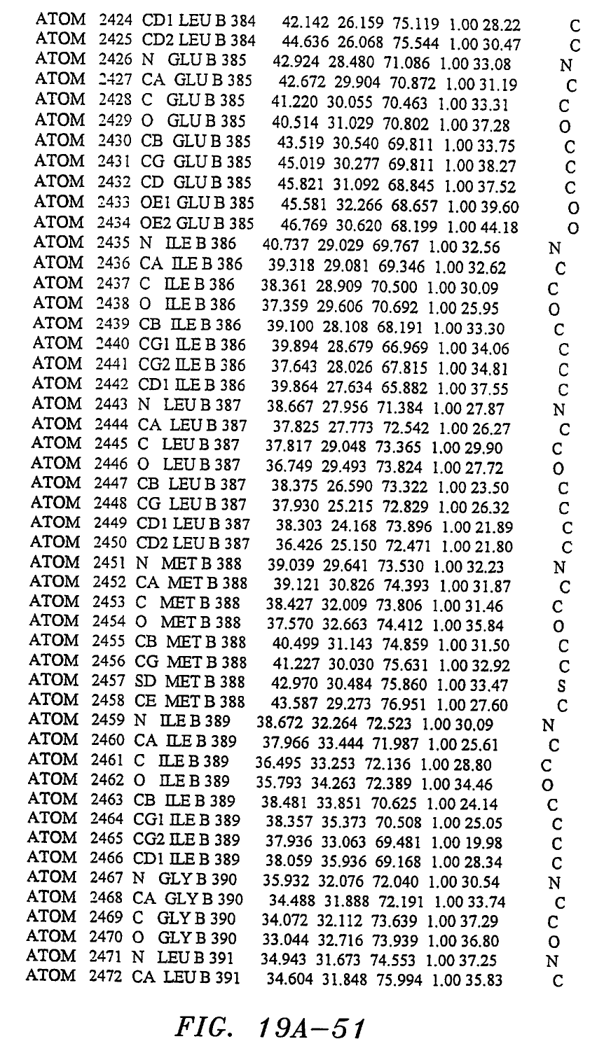

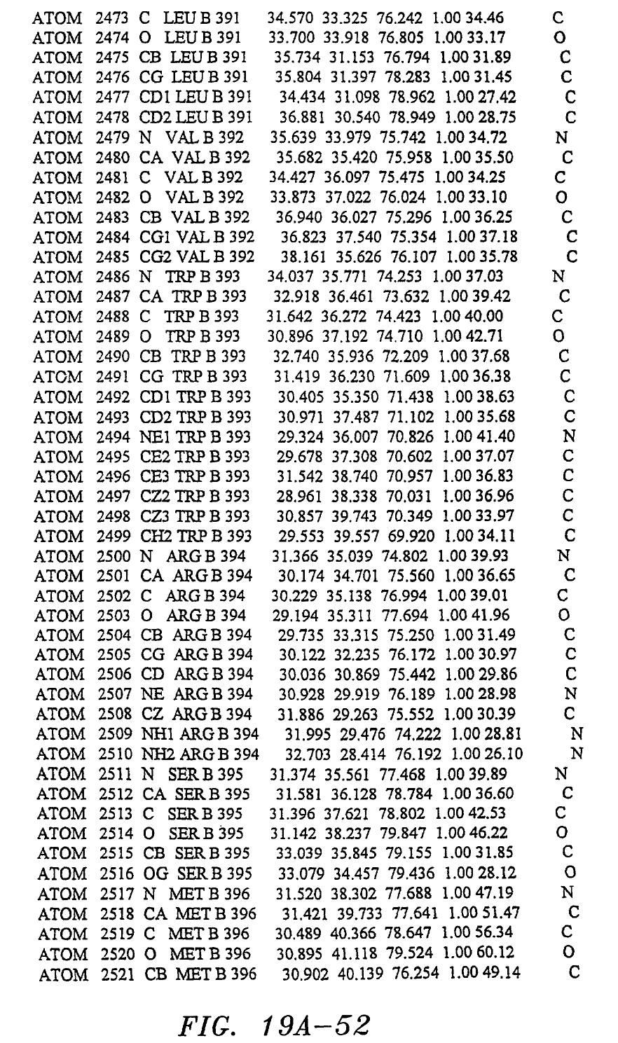

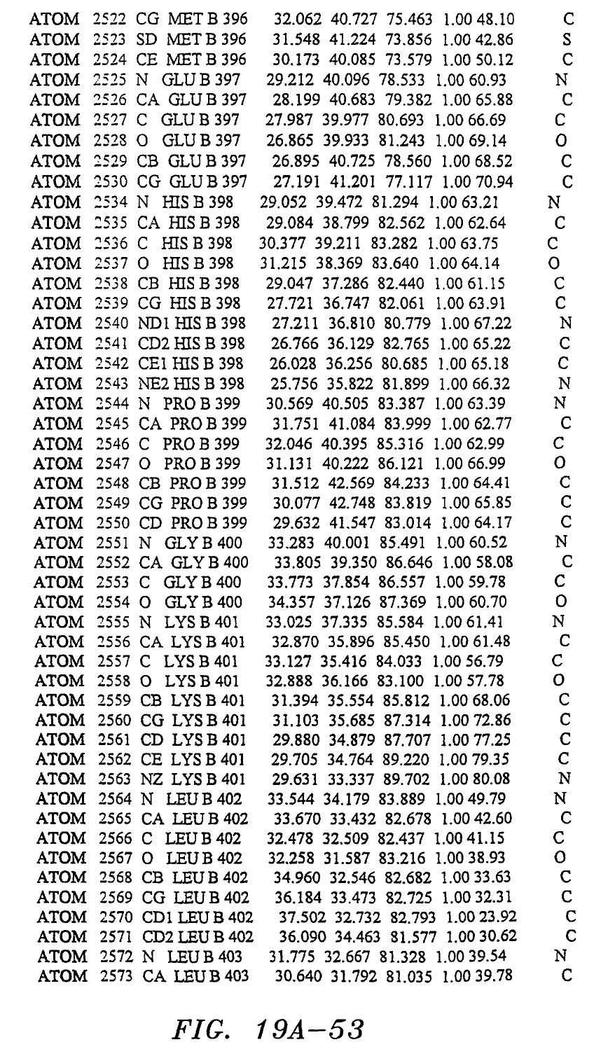

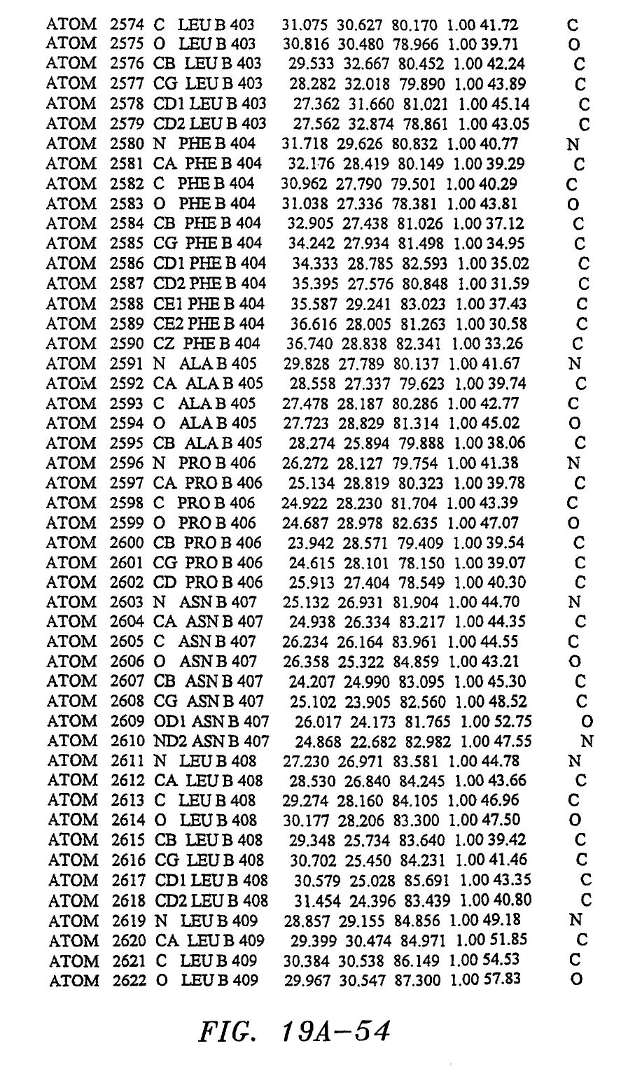

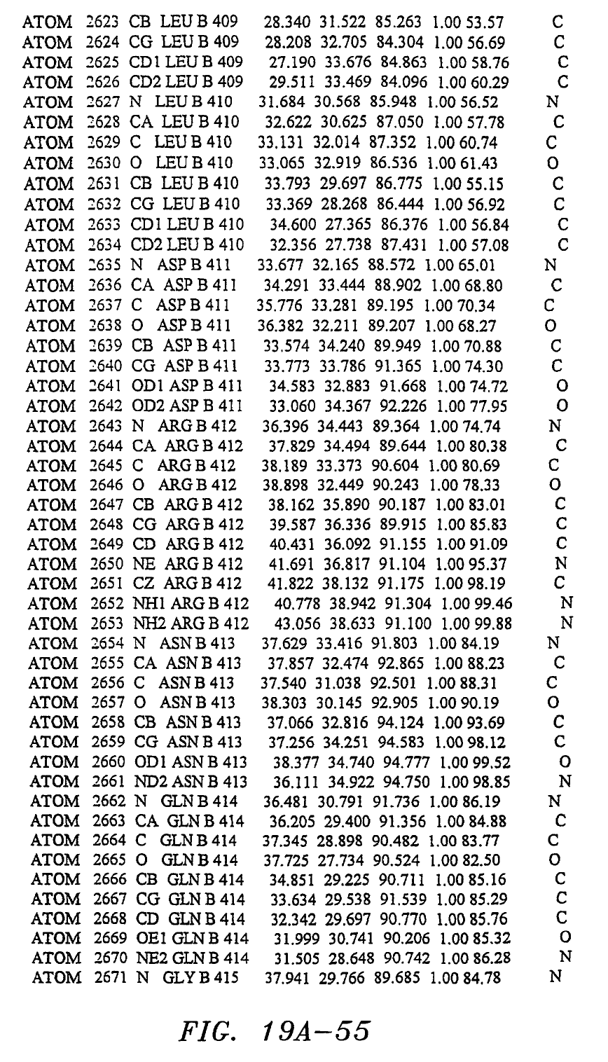

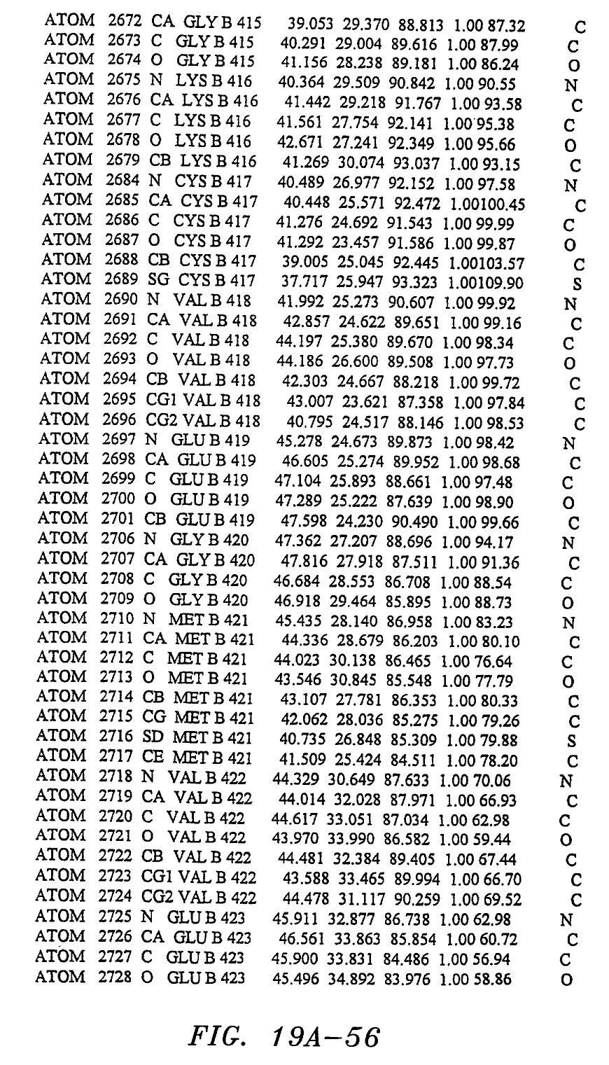

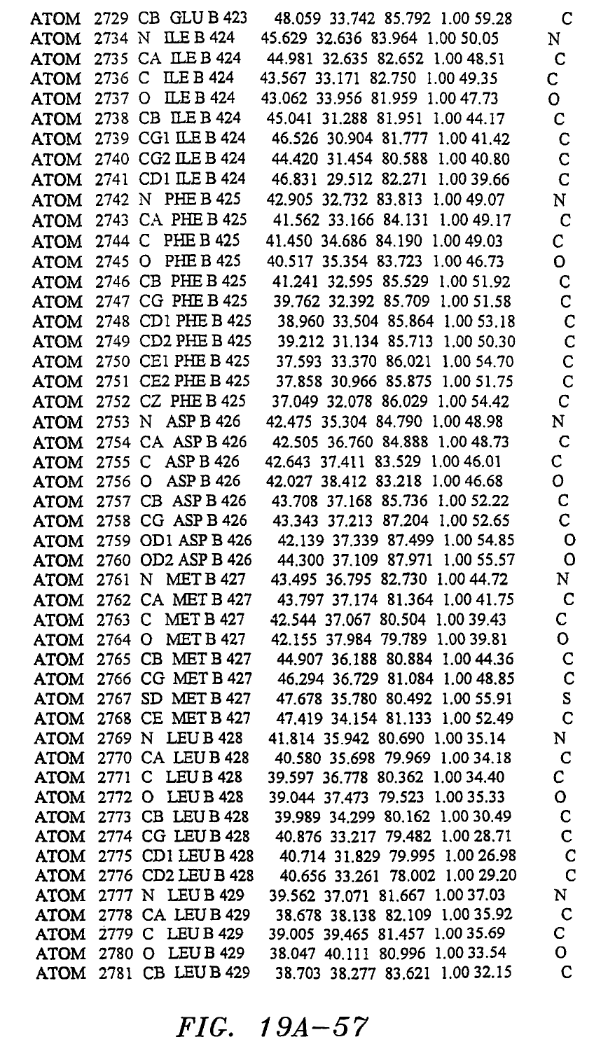

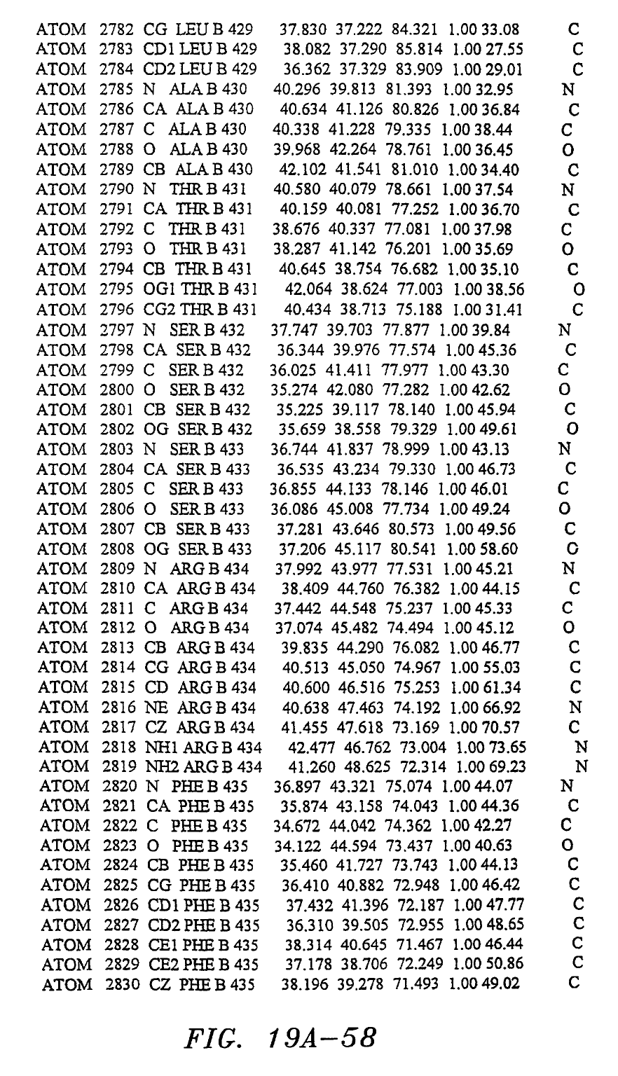

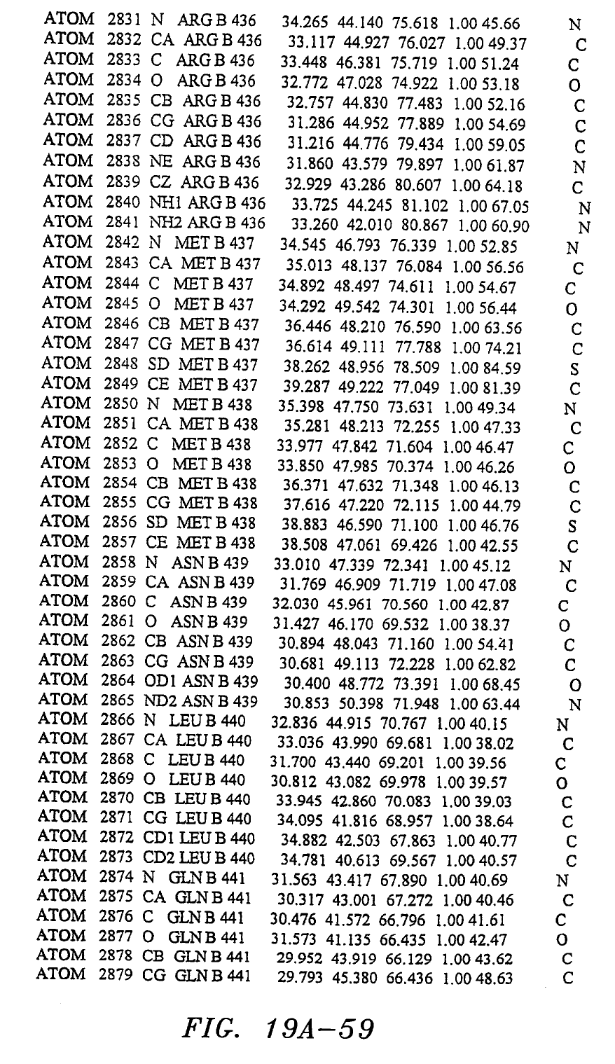

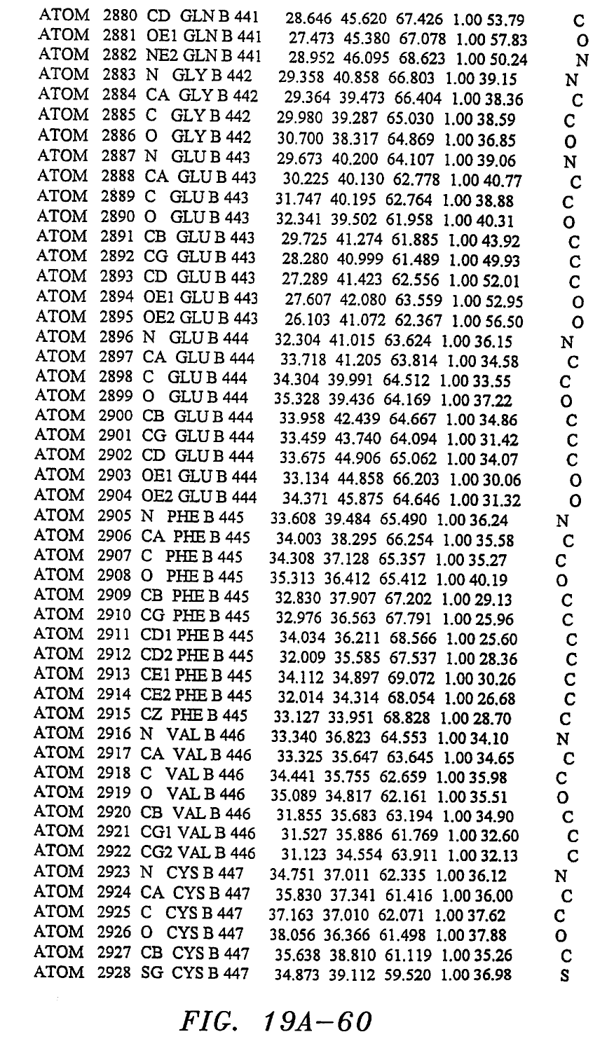

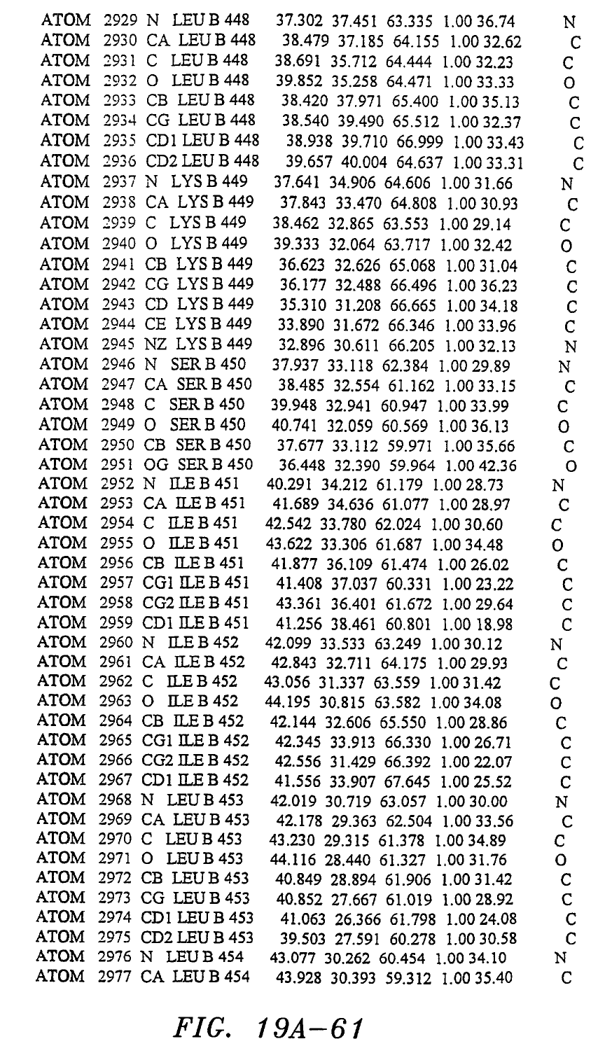

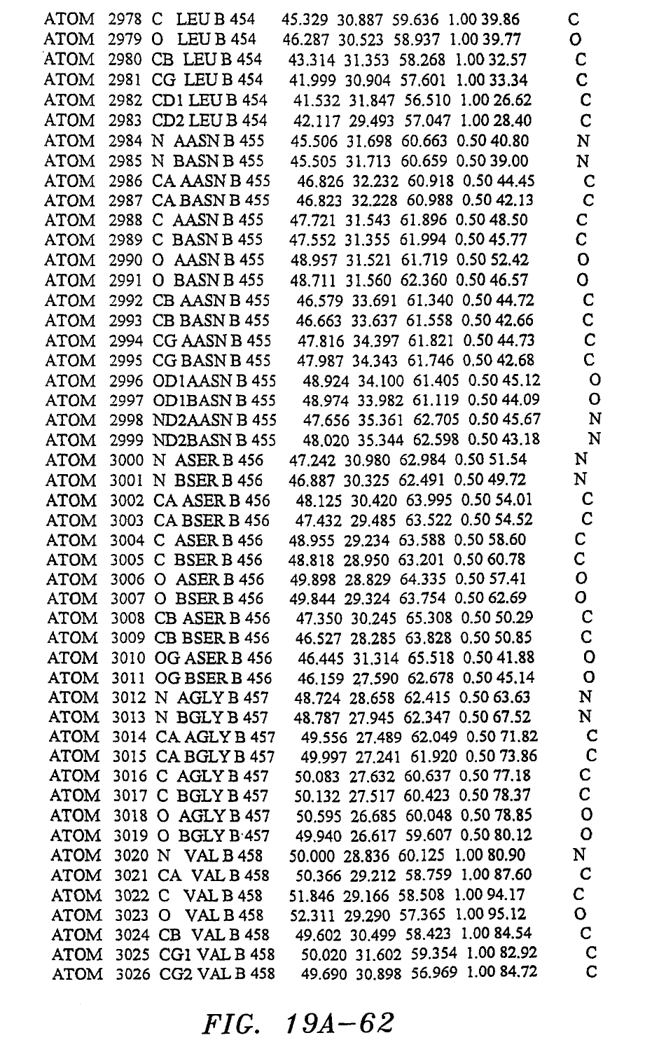

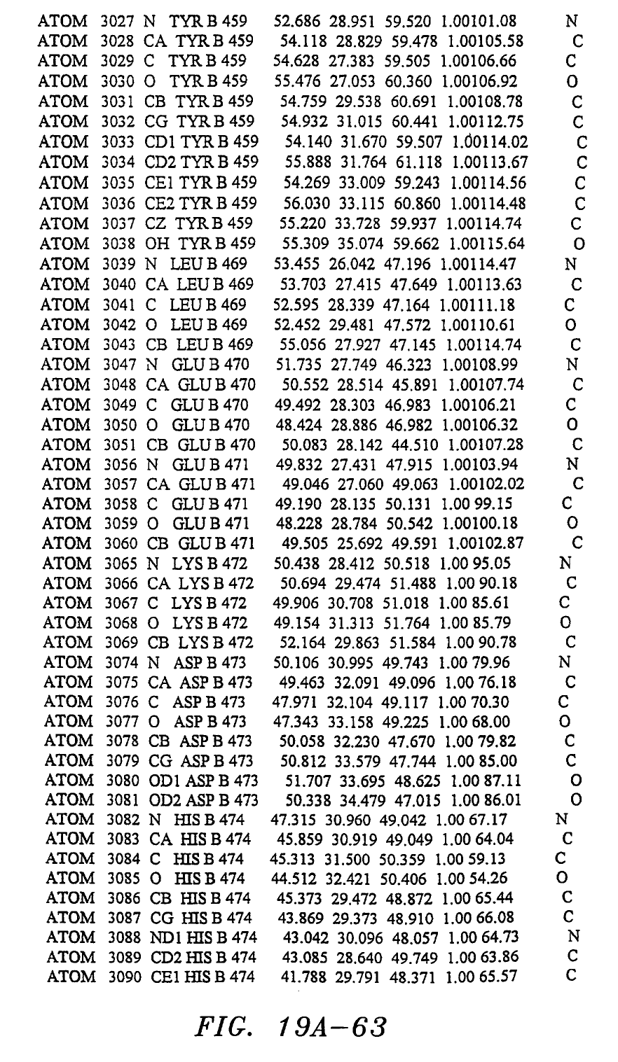

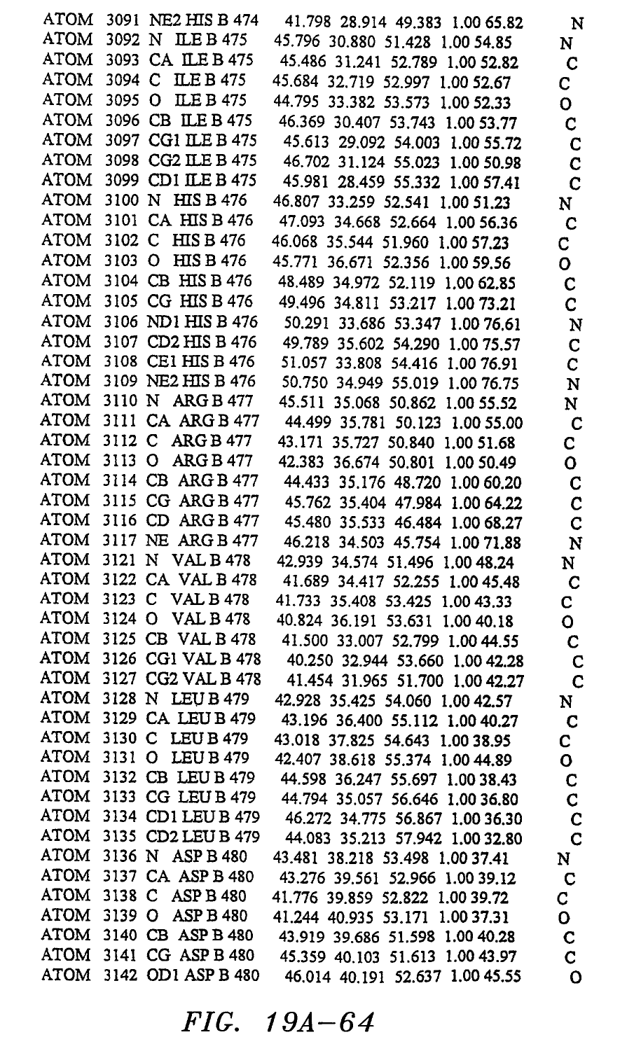

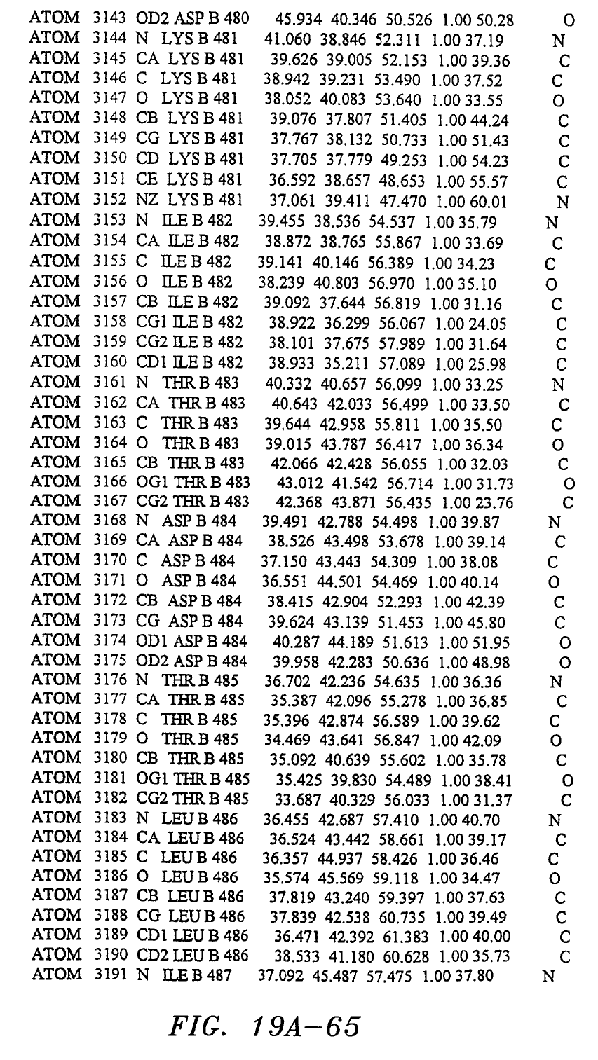

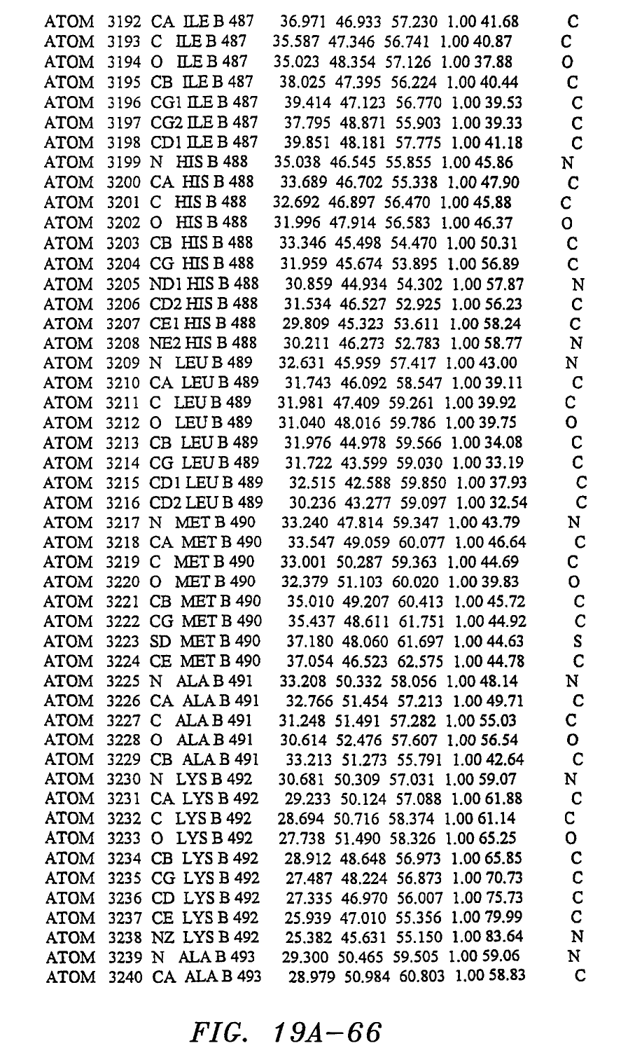

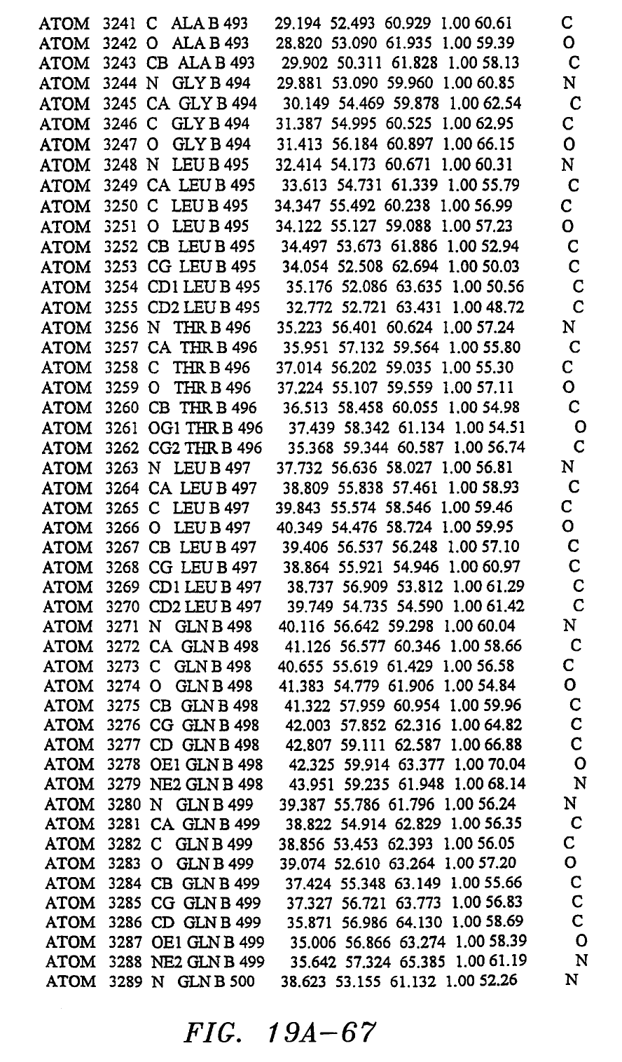

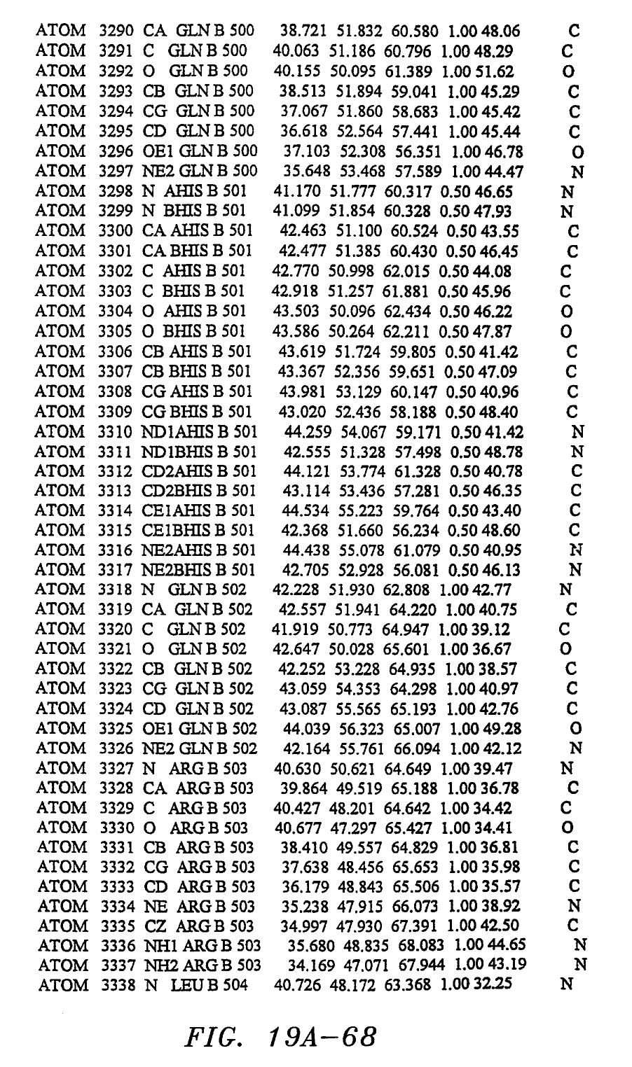

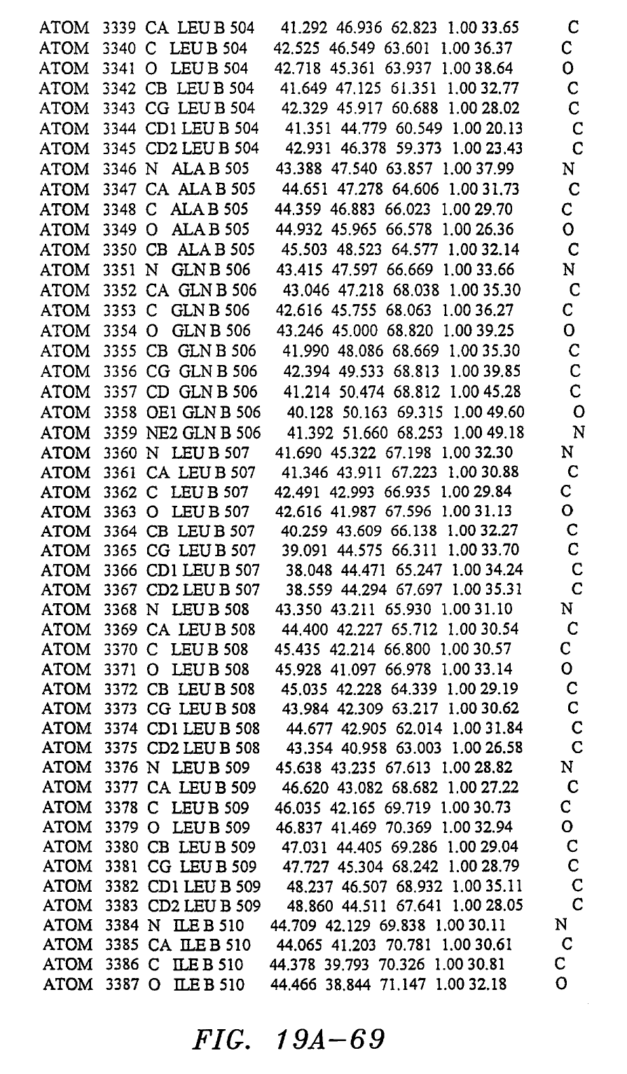

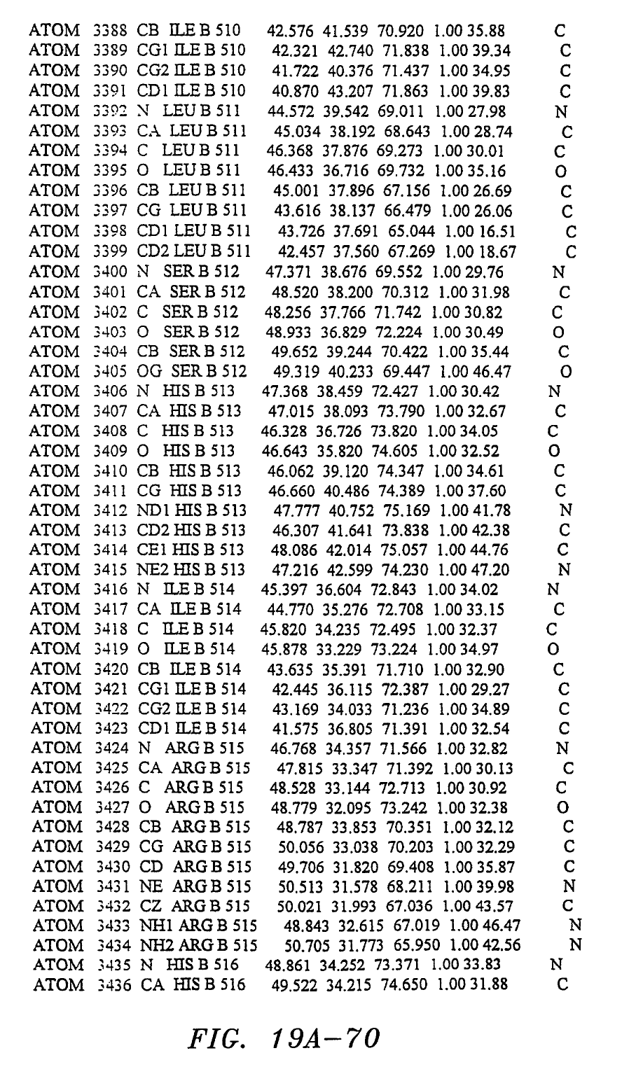

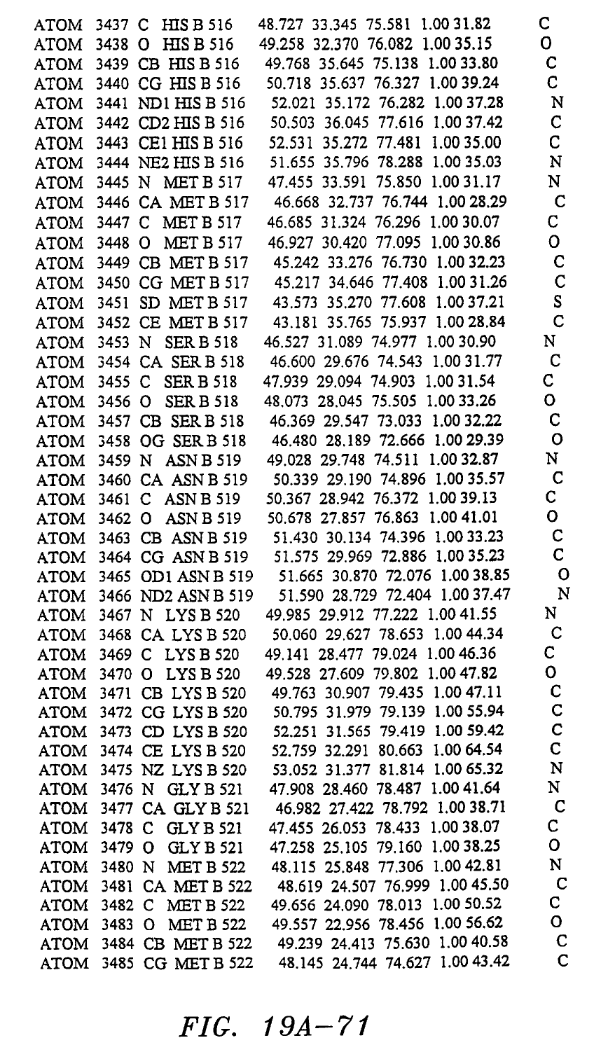

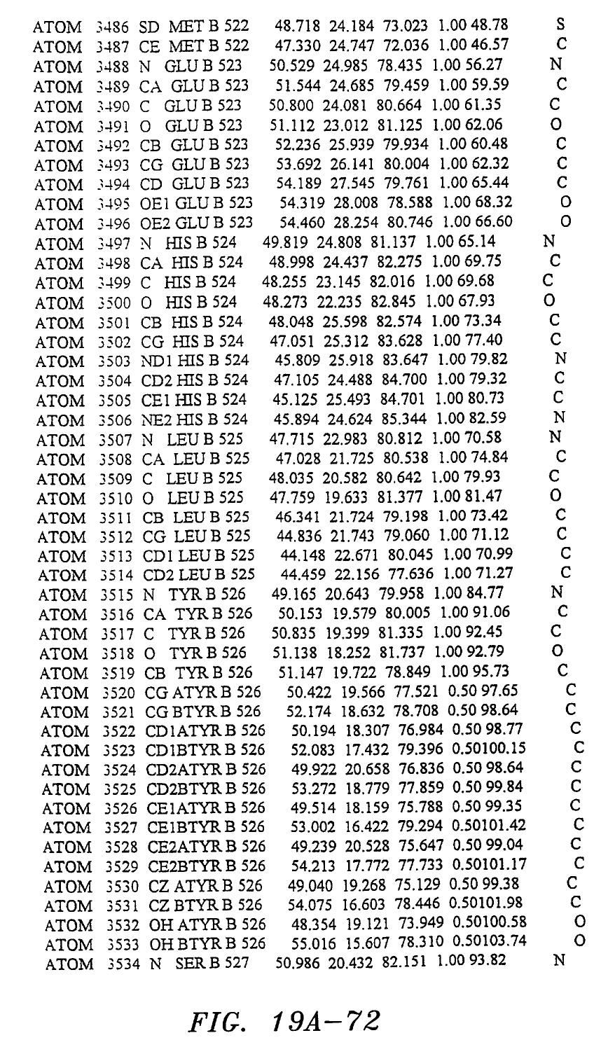

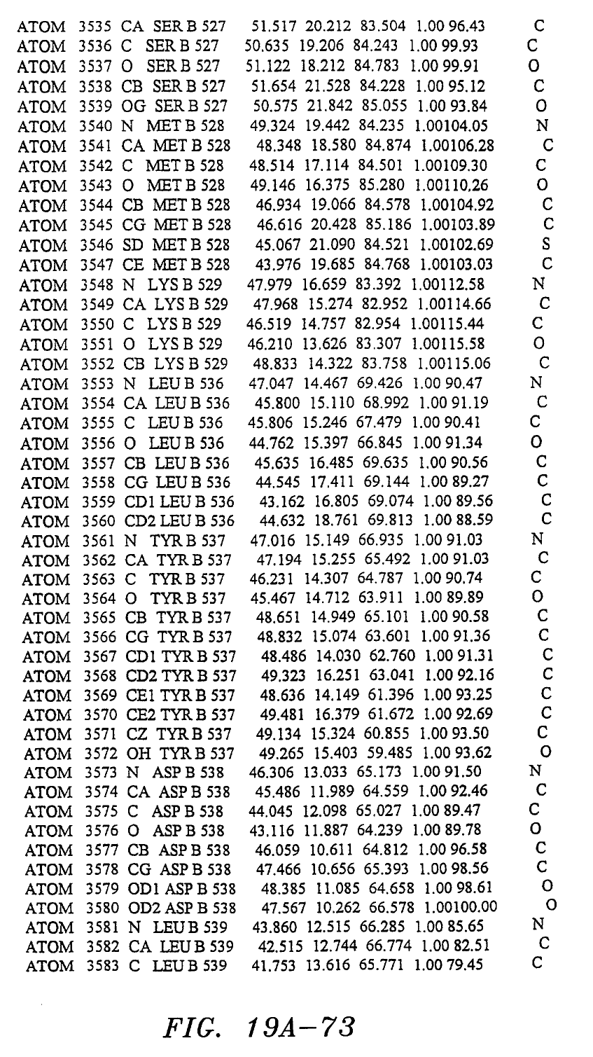

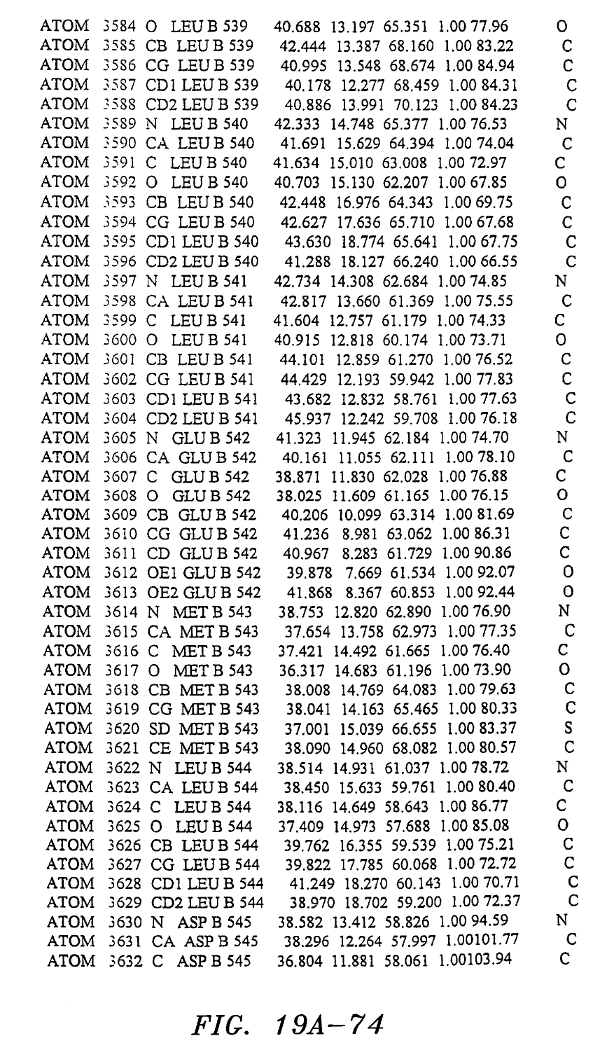

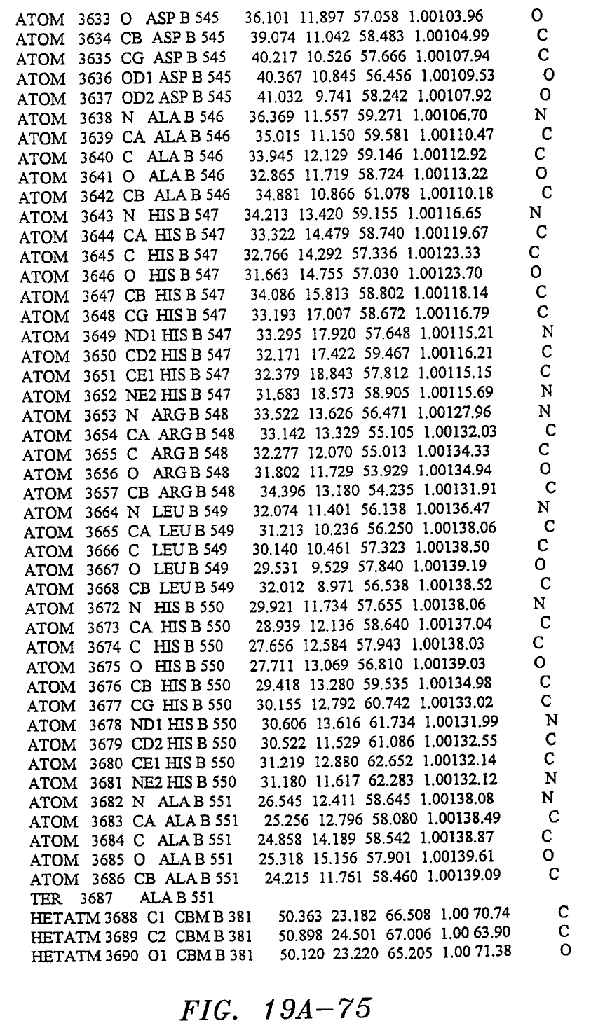

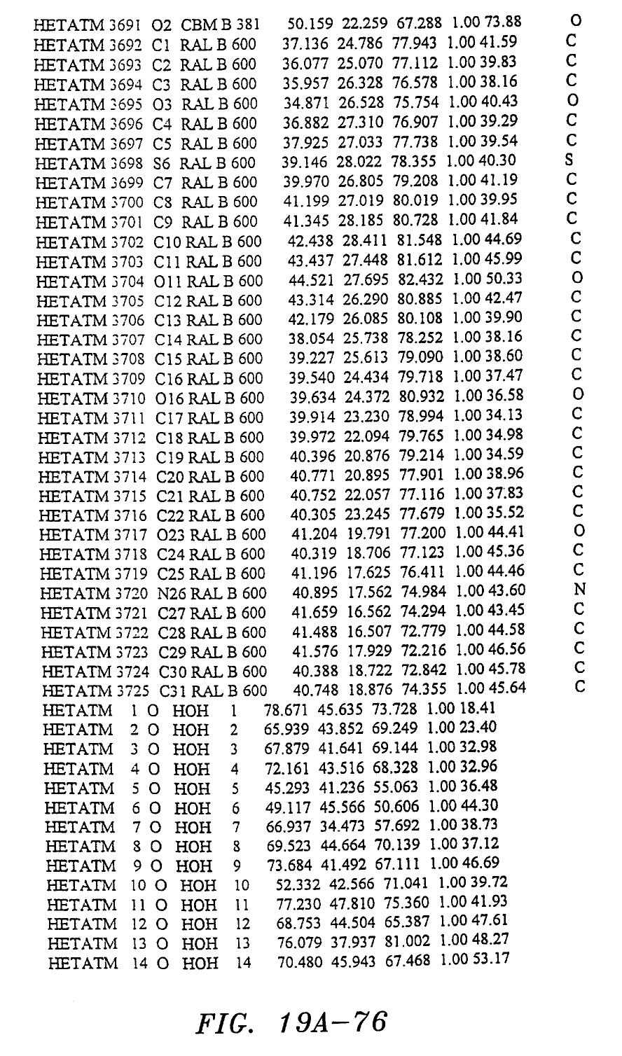

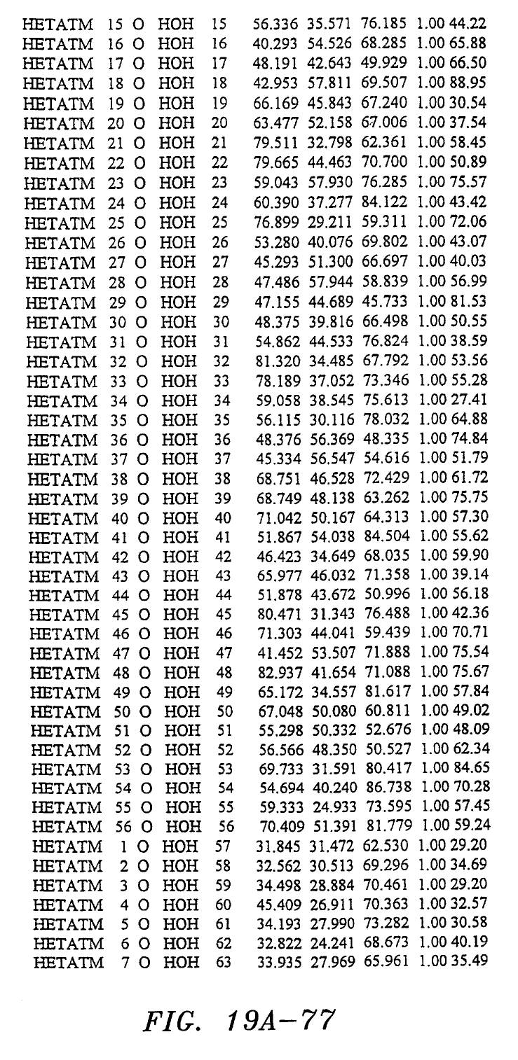

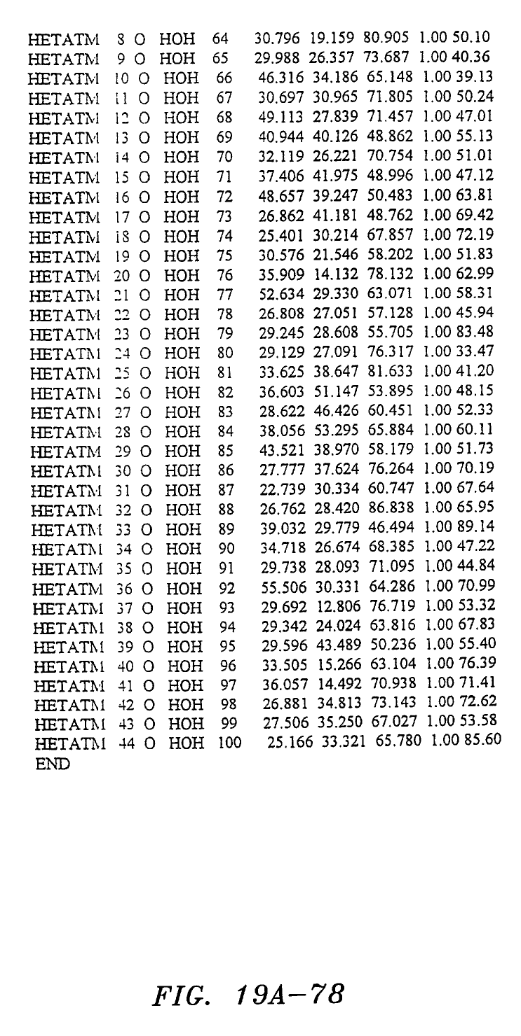

Embodiments of the invention will now be described in more detail, by way of example only, with reference to the accompanying drawing FIGS. 1 to 23 of which:

FIG. 1a shows representative portions of a 2.6 Å resolution multicrystal averaged map for a RAL-ER-LBD complex;

FIG. 1b is a 3.1 Å resolution six-fold averaged map for a E2-ER-LBD complex. In both FIG. 1a and FIG. 1b, the map is contoured at 1F and superimposed on the final, refined models;

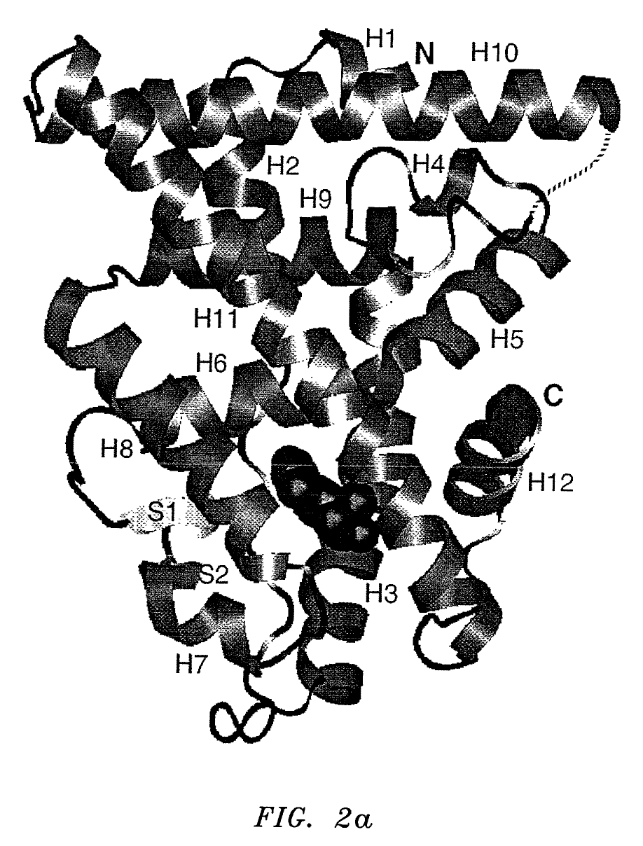

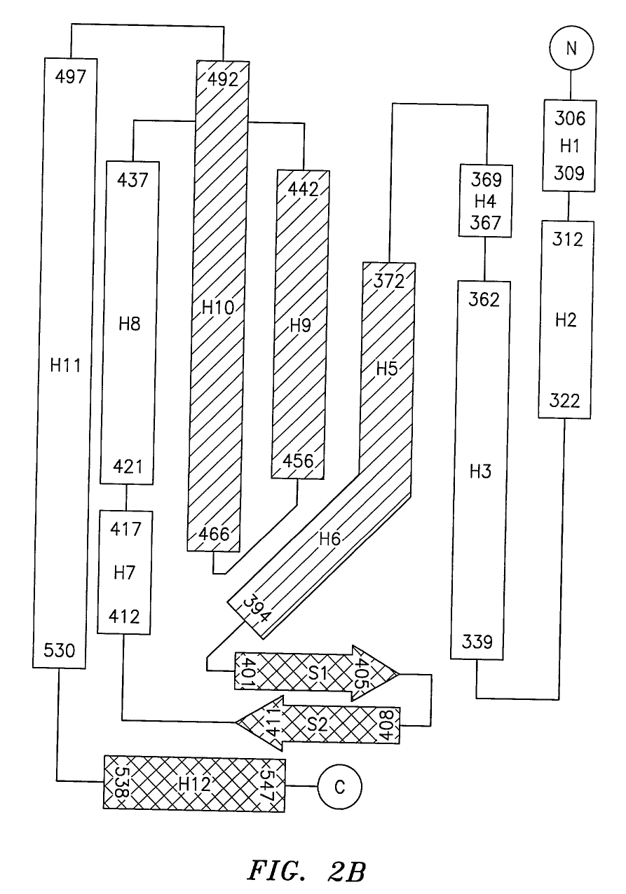

FIG. 2a is a schematic representation of the ER-LBDa indicating the locations of the various secondary structural elements″ and 3₁₀ helices are coloured grey, extended regions are very light grey and coil regions are coloured in dark grey. E2 is coloured very dark grey and is highlighted in space-filling form;

FIG. 2b is a topology diagram for ER-LBD. Helices are represented as rectangles and β strands as arrows. The central core layer (H5,H6,H9 and H10—striped) is sandwiched between the outer flanking layers (H1-4) (H7, H8, H11). The structural elements which flank the layered motif (S1/S2 and H12) are S1, S2, H12 and are cross hatched. The N and C termini are also labelled. All secondary structural elements have been numbered in keeping with the nomenclature that has been established for other known nuclear receptor LBDs;

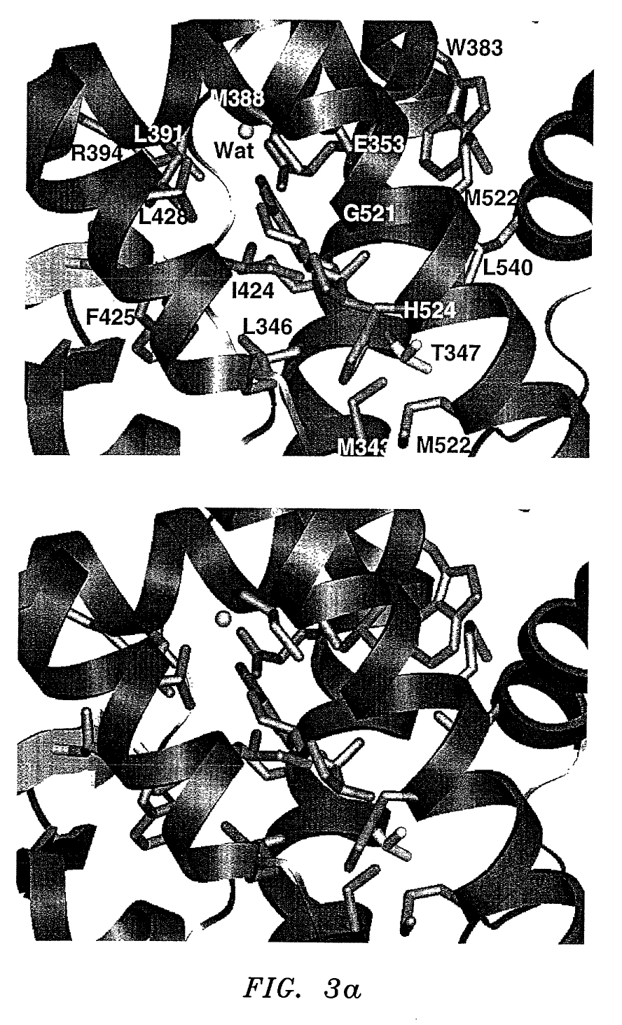

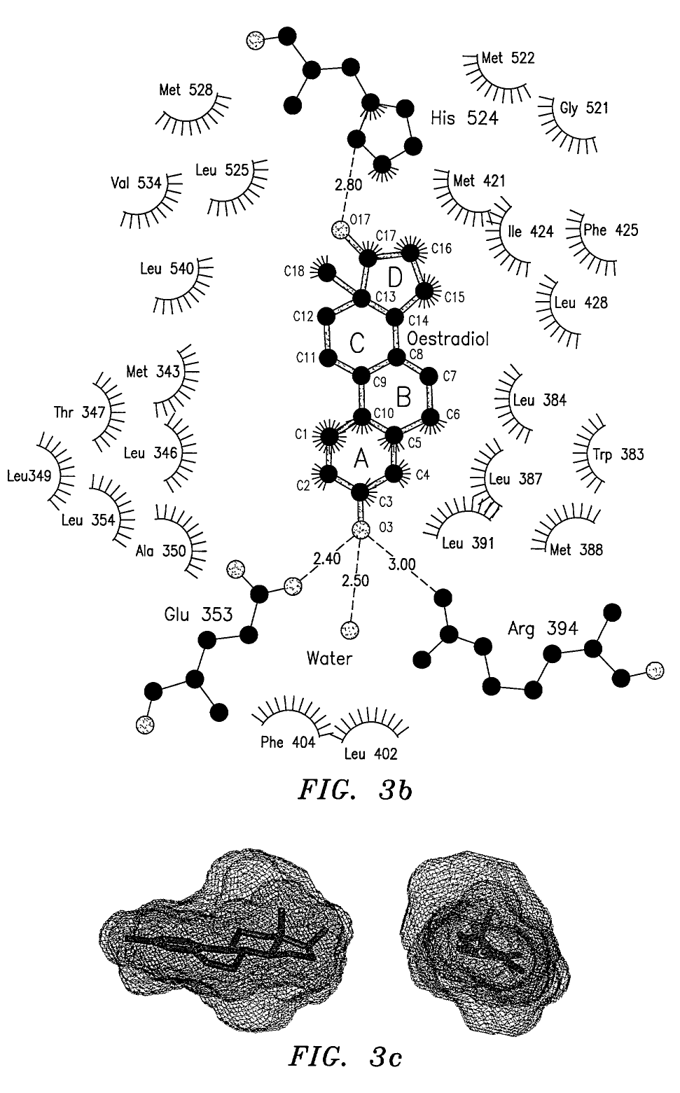

FIG. 3a is a stereoview of the ligand binding cavity. The cavity is viewed in a similar orientation to that given in FIG. 1a. Sidechains for residues that line the cavity are illustrated. Hydrophobic residues are shown in grey, basic residues are shown as spotted and acidic residues are shown in hatched. E2 is coloured black (core) and dark grey (terminal hydroxyl groups);

FIG. 3b is a schematic representation of the ligand binding cavity. Residues that make direct hydrogen bonds to the hydroxyl radius are shown in ball-and-stick representation along with hydrophobic residues that make non-polar interactions with E2 (shown as grey with radial spokes). The atom names and ring nomenclature of E2 are also given;

FIG. 3c is a representation of the molecular volume of E2 (dark grey dotted surface) and the accessible binding cavity volume (light grey dotted surface);

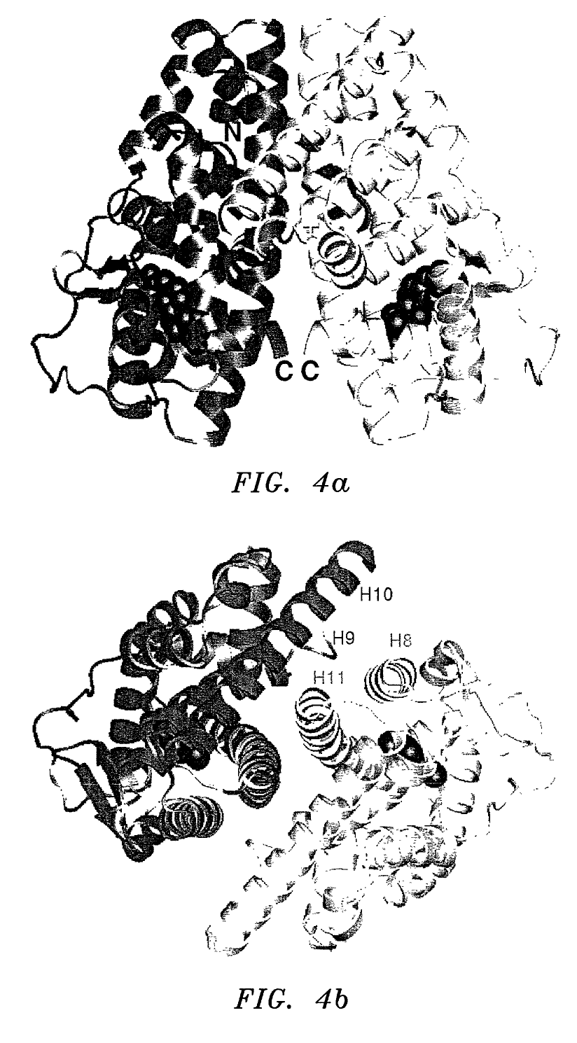

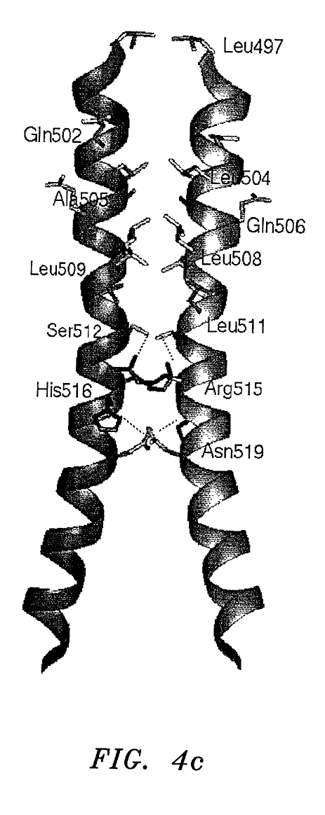

FIG. 4a is a schematic representations of the ER-α LBD non-crystallographic dimer viewed perpendicular to the dimer axis. The N and C termini are labelled;

FIG. 4b is a view of the dimer of FIG. 4a along the dimer axis. E2 is highlighted in mid grey in space-filling form. Helices that are involved in the dimer interface are labelled;

FIG. 4c is a view showing the H11 helices that form the backbone of the dimer interface. Interacting residues are show coloured according to polarity (grey —hydrophobic residues; hatched—polar residues; cross-hatched—basic residues);

FIG. 5a is a schematic representation of the binding cavity and interactions made by E2. The figure was produced using LIGPLOT software;

FIG. 5b is a comparison of the E2 and RAL binding modes (E2—dark grey; RAL—light grey);

FIG. 6 is a schematic representation of the ER-LBD showing the different positioning of helix 12 in the E2 (cross-hatched) and RAL (hatched) complexes. The remainder of the ER-LBD is shown in grey. Dashed lines indicate unmodelled regions of the structure. The helices which interact with H12 in the two complexes are marked; and

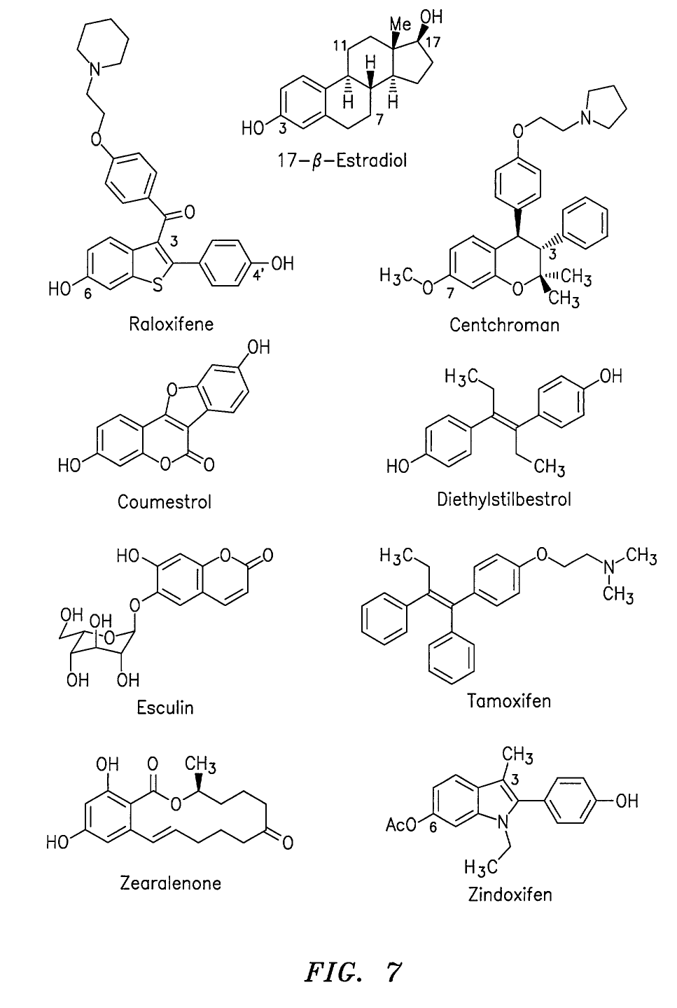

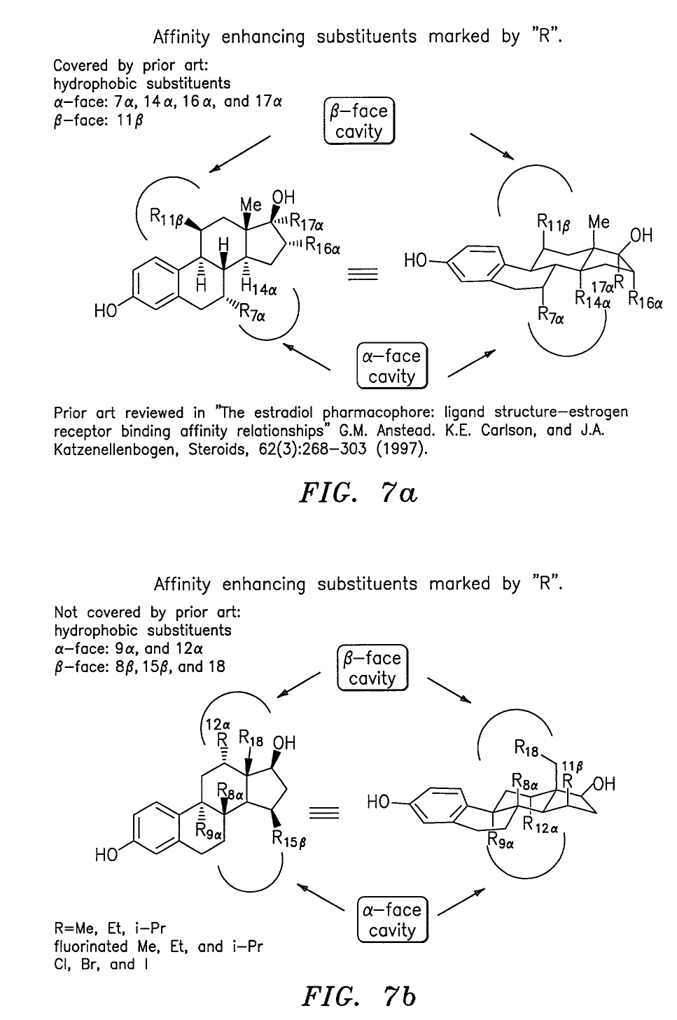

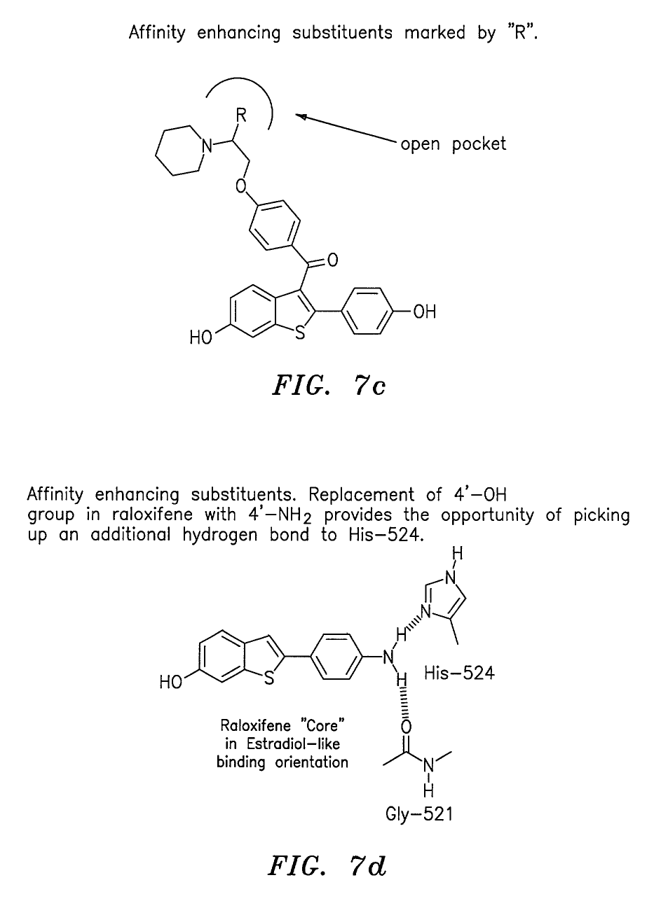

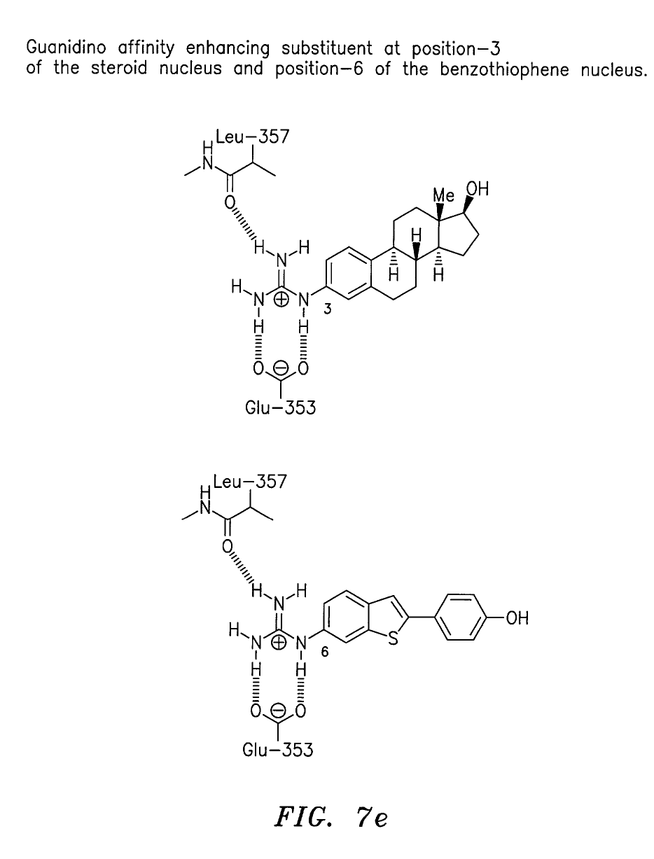

FIG. 7 is a space filling representation of a) an E2 complex and b, an RAL complex. H12 (black) is positioned over the hormone binding cavity in the E2 complex. Raloxifene induces a conformational change so that H12 occupies a hydrophobic groove between H3 and H5. The hydrophobic sidechains of all residues that lie between residues 354 (H3) and 380 (H5) are drawn in dark grey. Other highlighted residues are Lys362 (hatched), Glu380 and Tyr537 (cross-hatched), Asp351 (spots) and the ligand RAL (grey). The remaining atoms of the LBD monomer are white. Note that differences in other parts of the ER-LBD complexes may be due regions missing from the current models;

FIG. 8 shows the structure of several representative estrogen receptor ligands;

FIGS. 8a, 8b and 8c show modifications made to the steroid nucleus of ligands which bind to the estrogen receptor;

FIGS. 8d and 8e show how affinity of the ligand can be enhanced by adding substituents; and

FIGS. 9a-9f show selectivity enhancement by using different substituents on the estrogen receptor ligand; and

FIGS. 10 to 19 show by way of structural formulae the chemical reactions involved in the following Examples 1 to 51, which are non-limiting and given by way of illustration only.

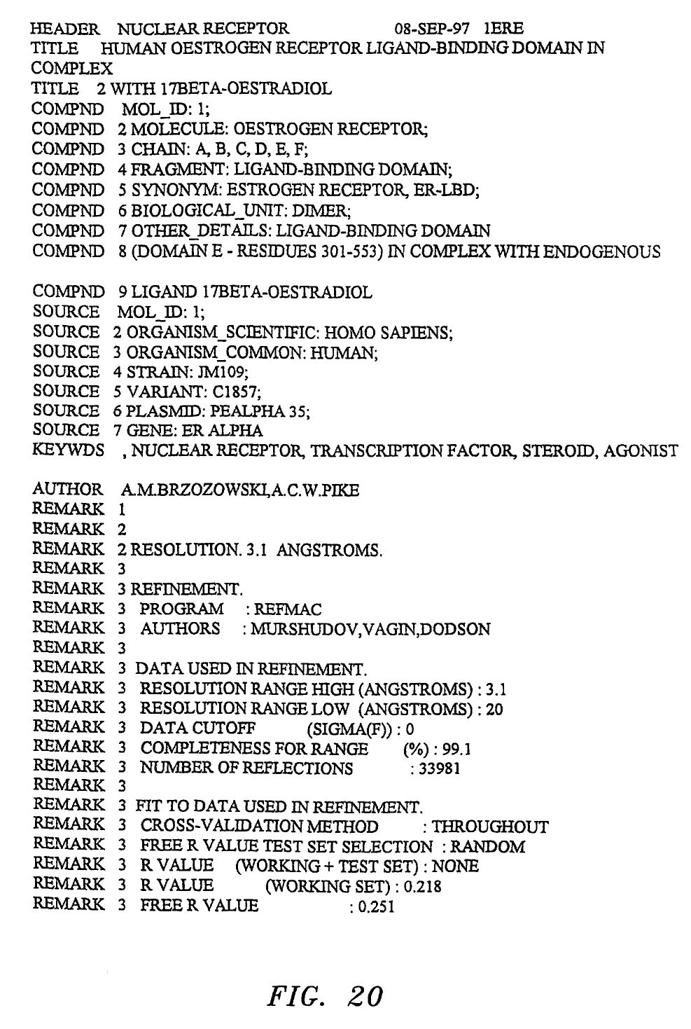

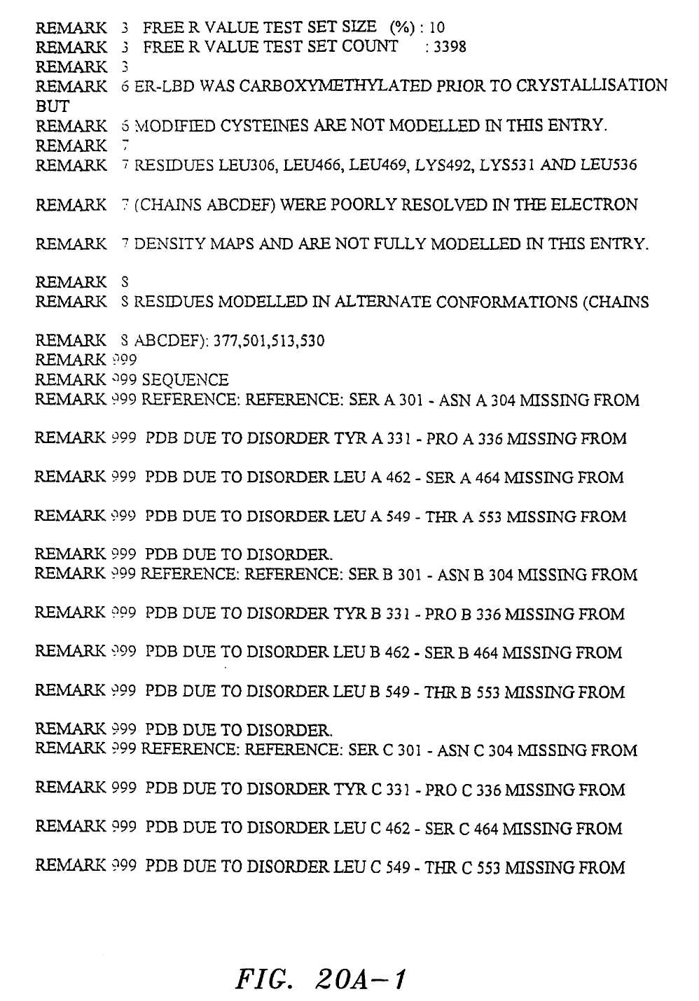

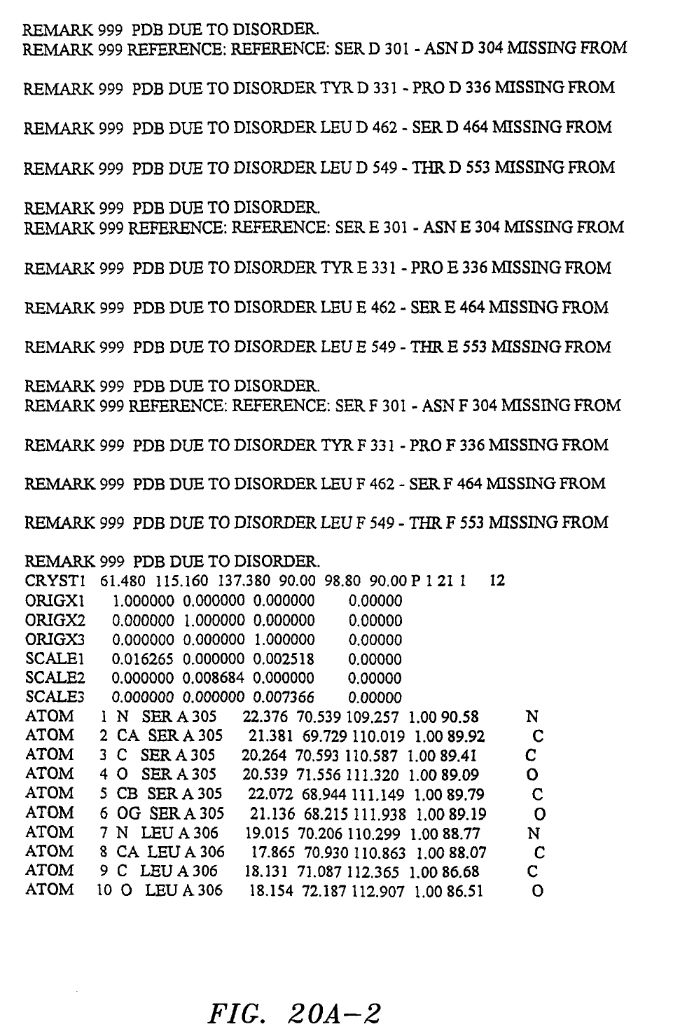

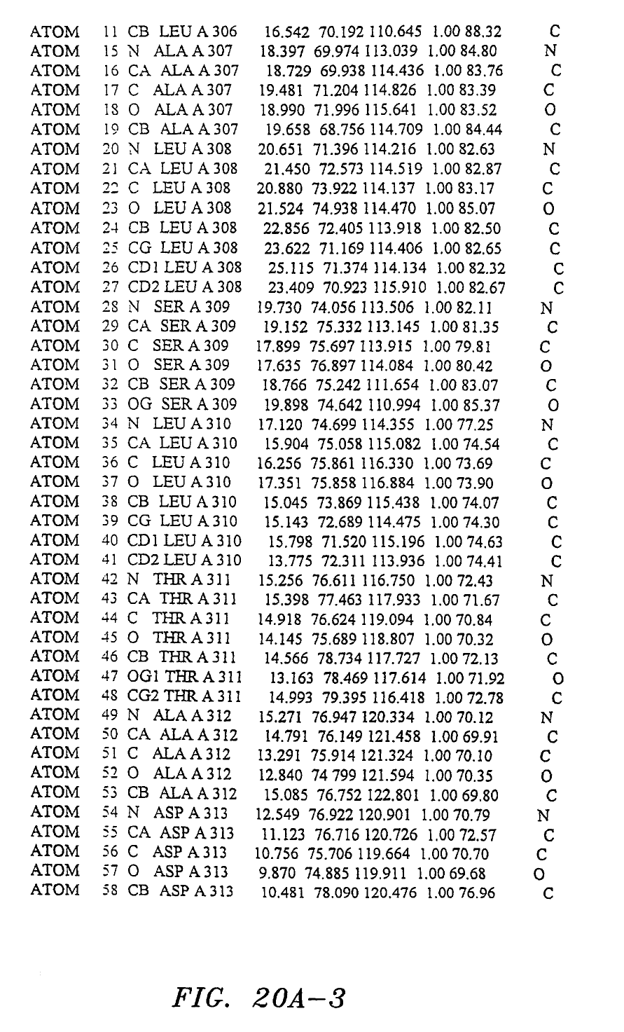

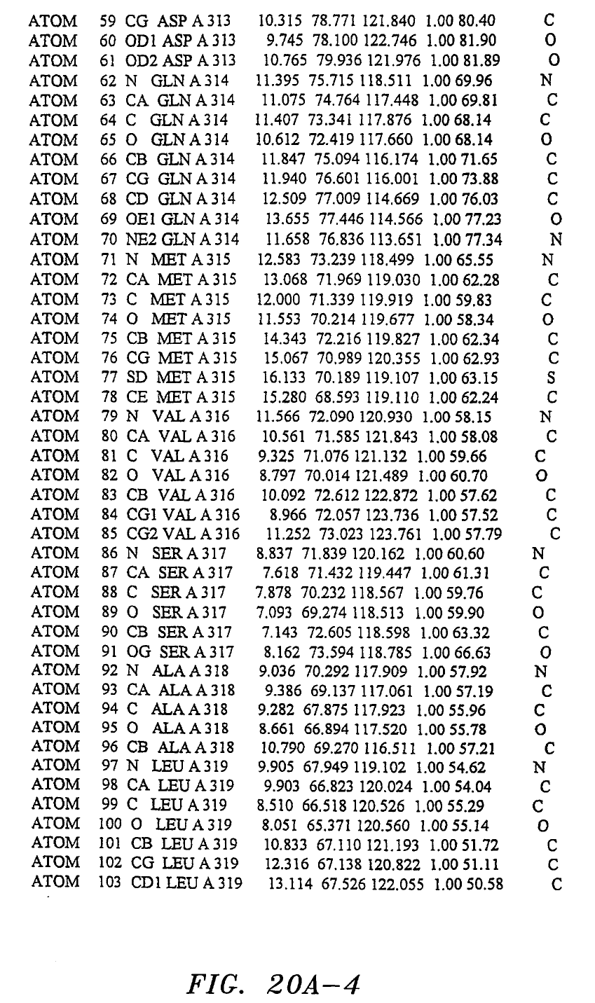

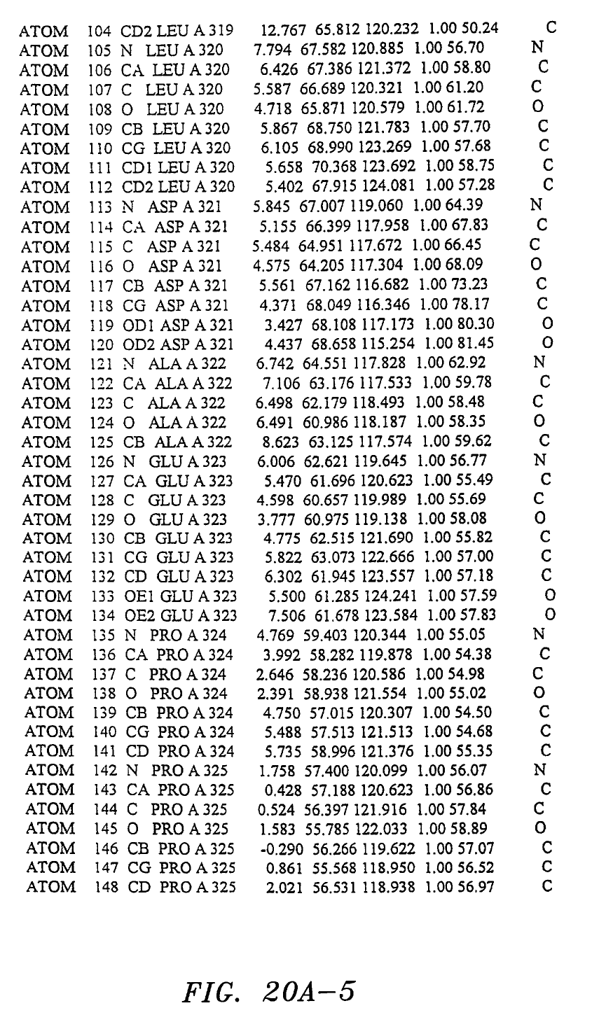

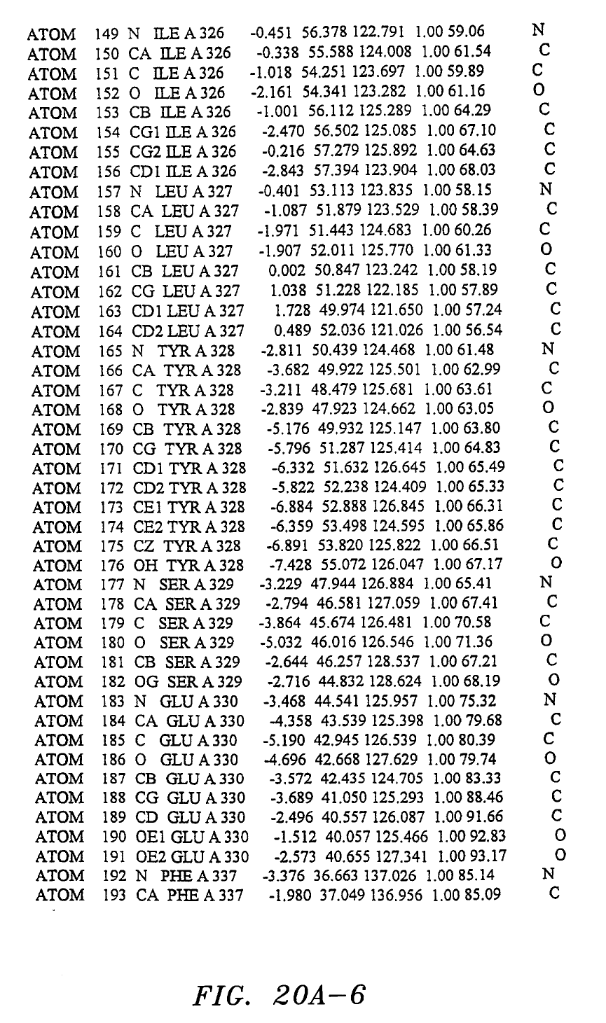

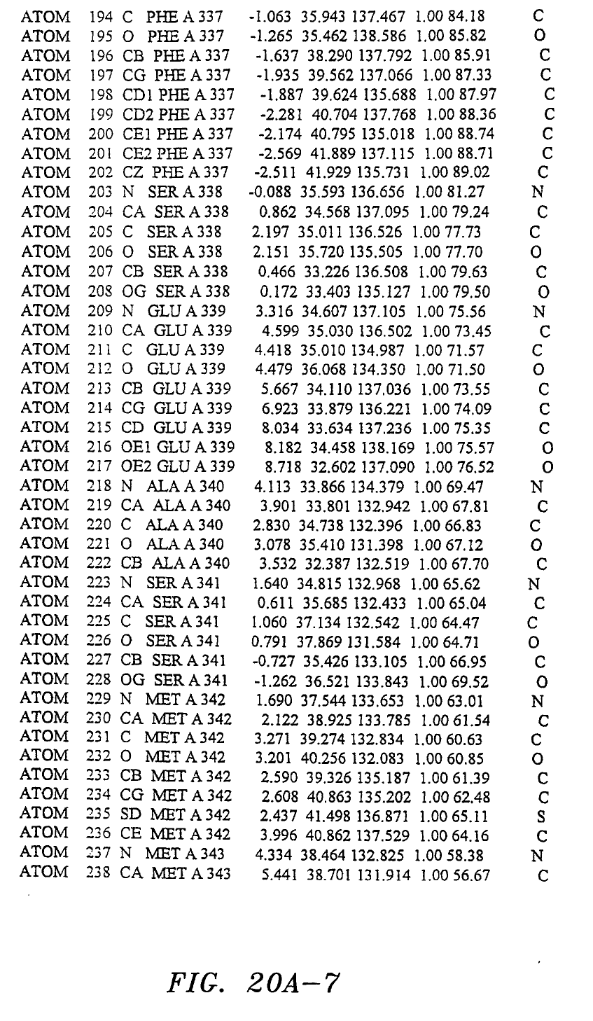

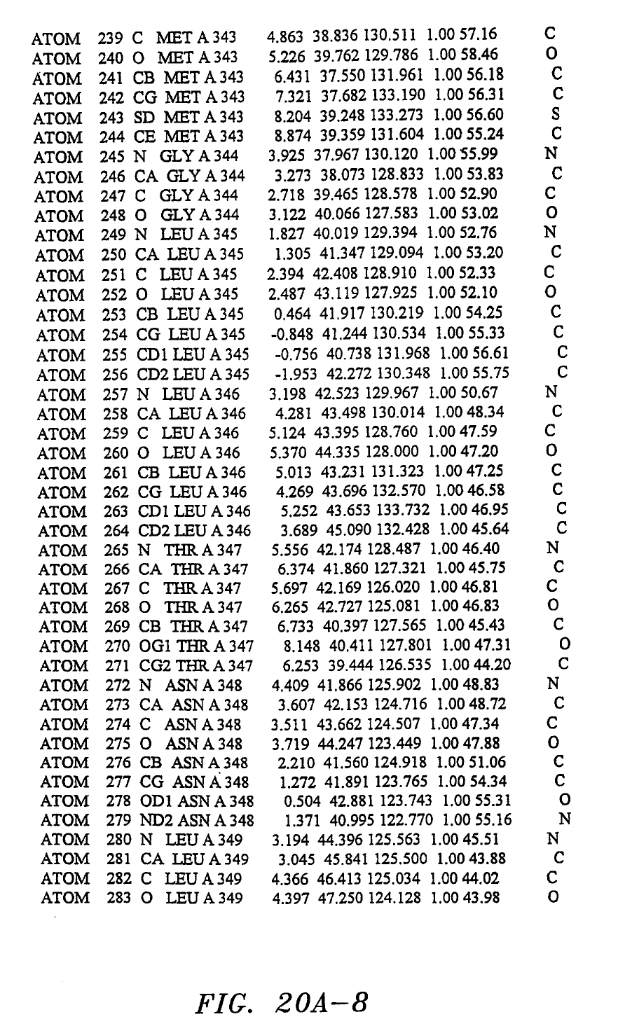

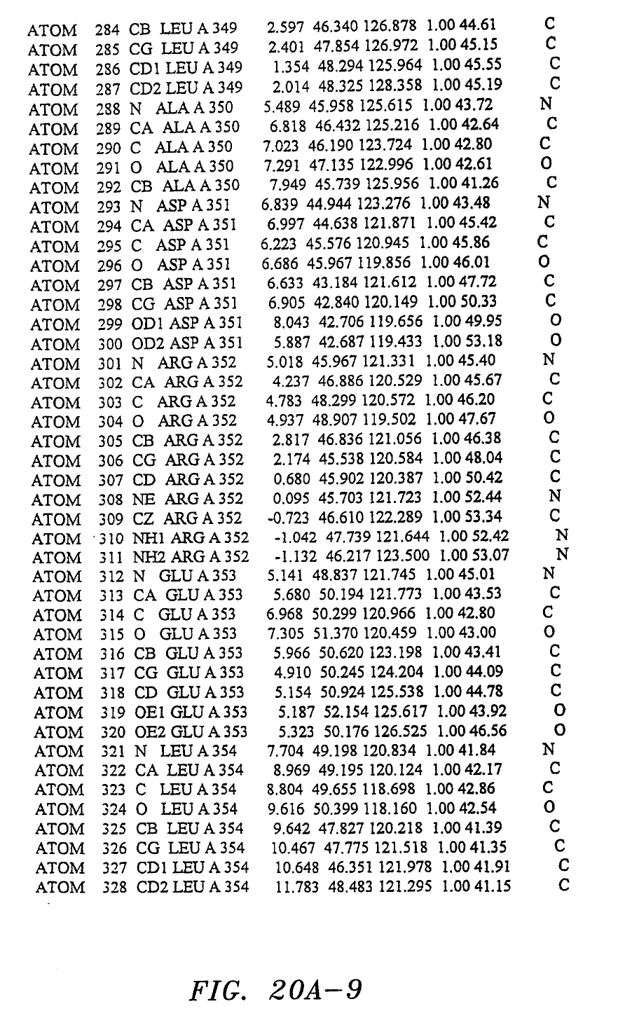

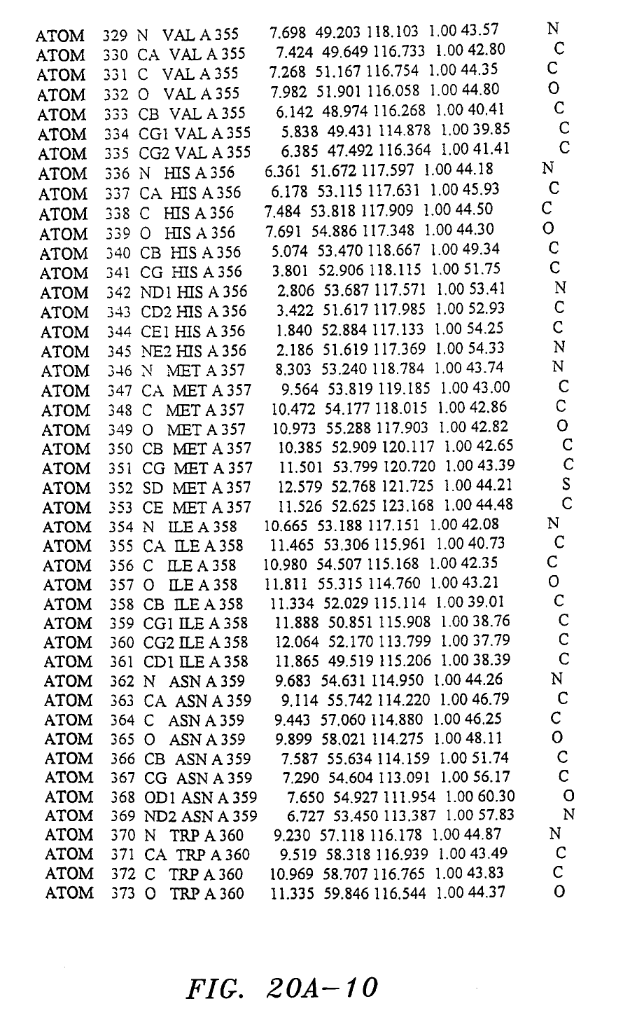

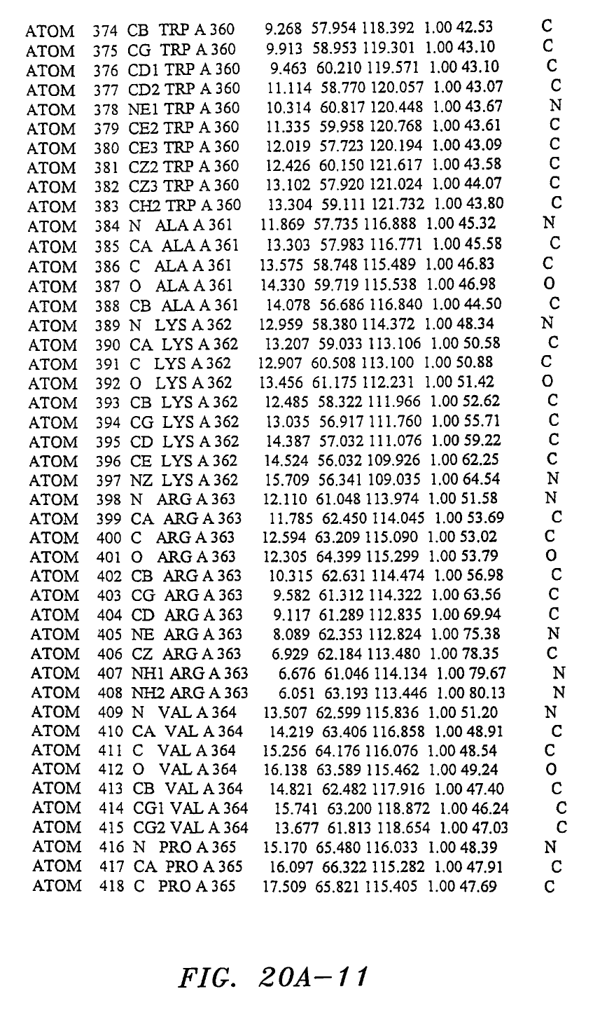

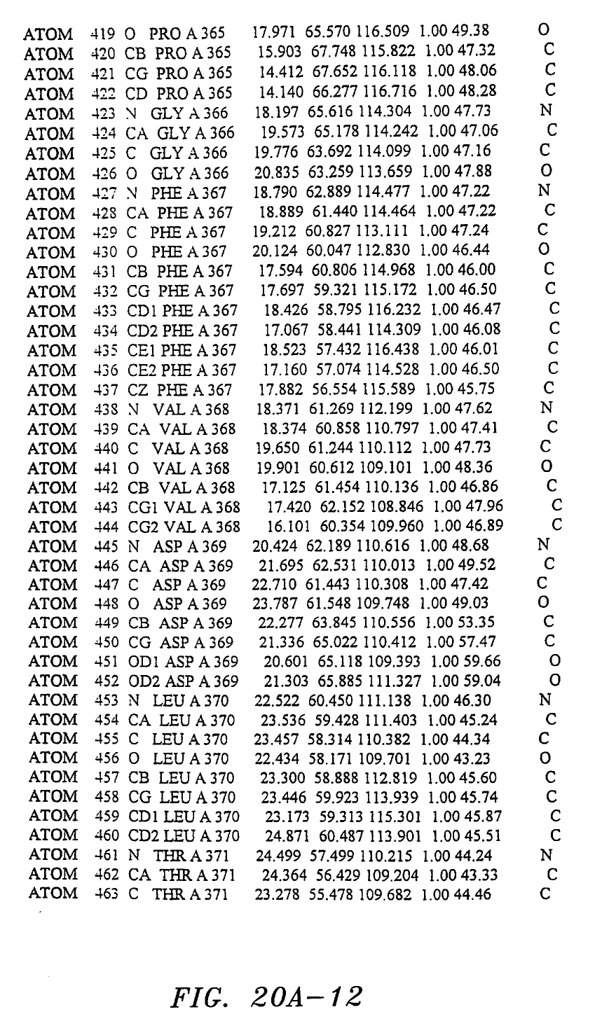

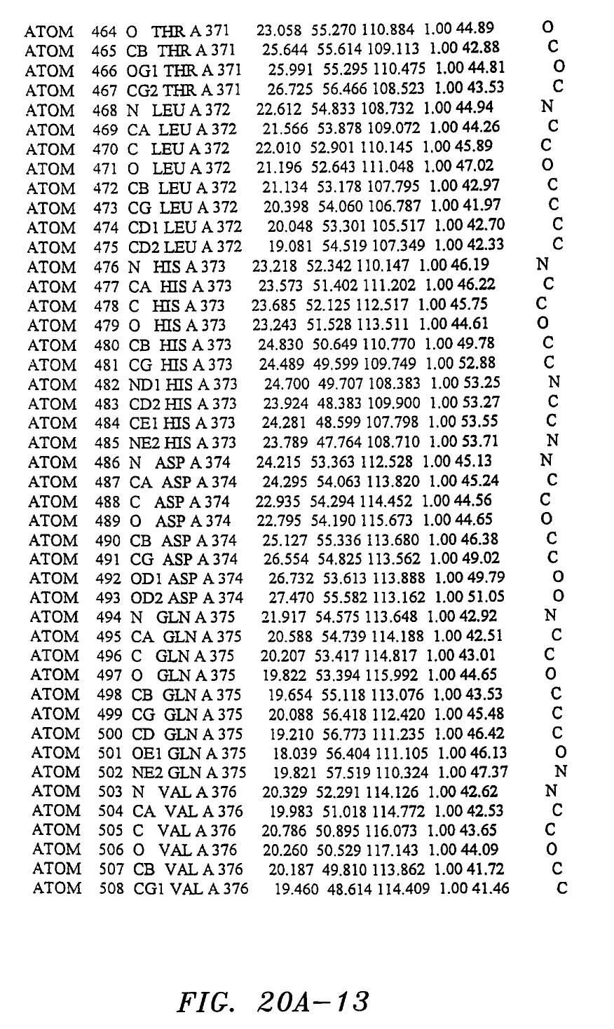

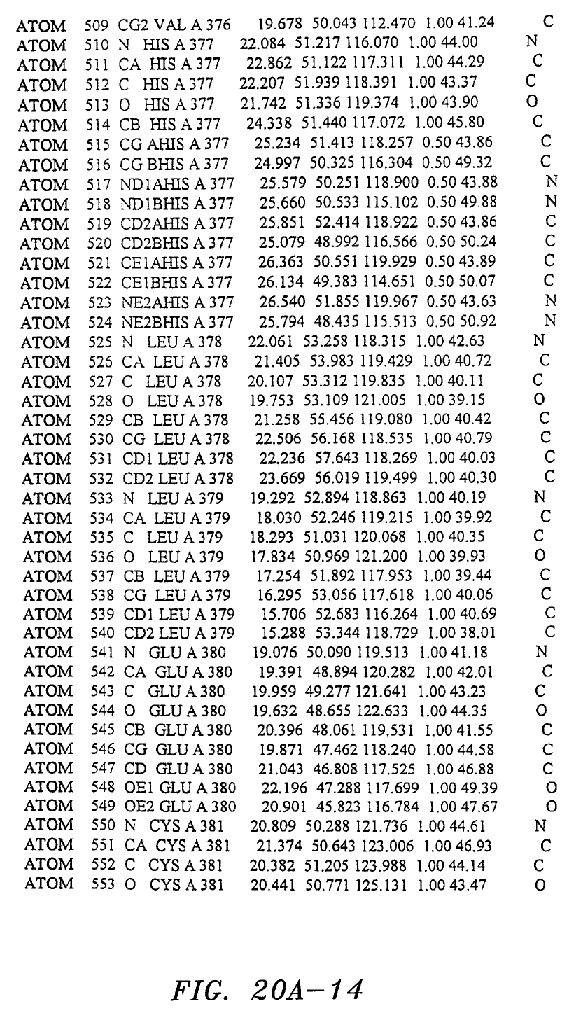

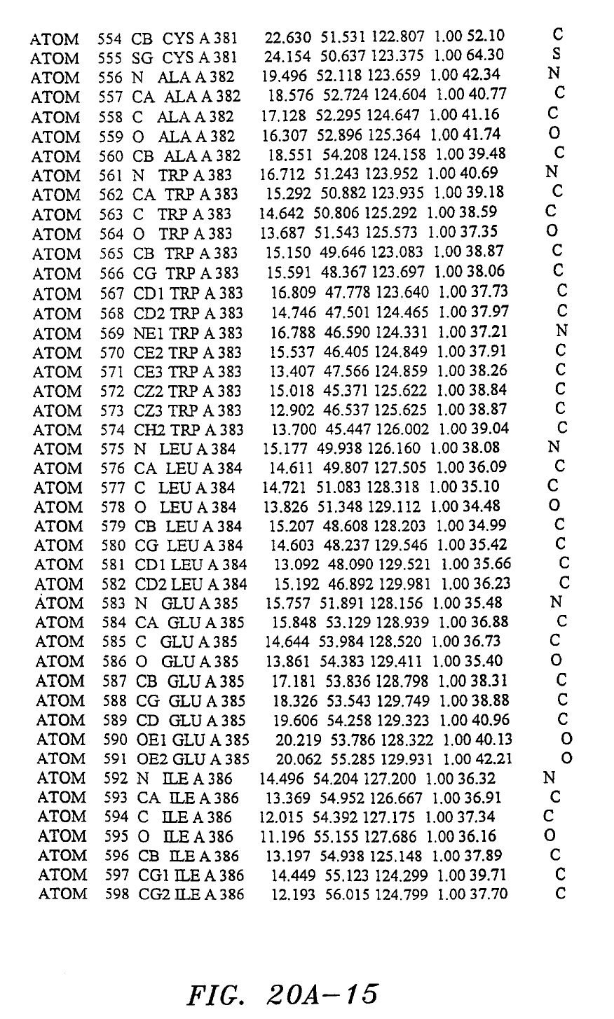

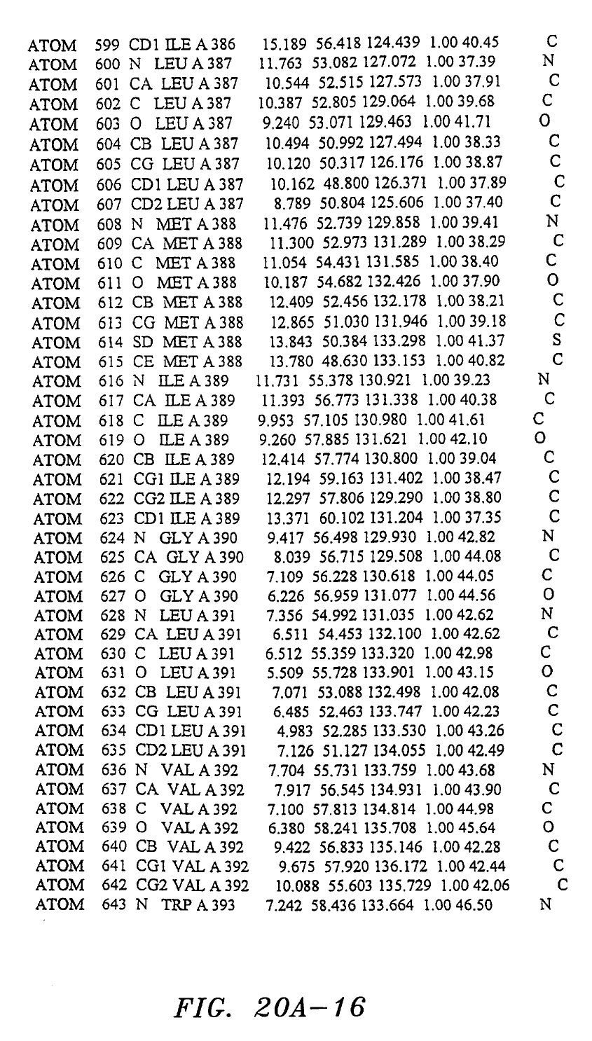

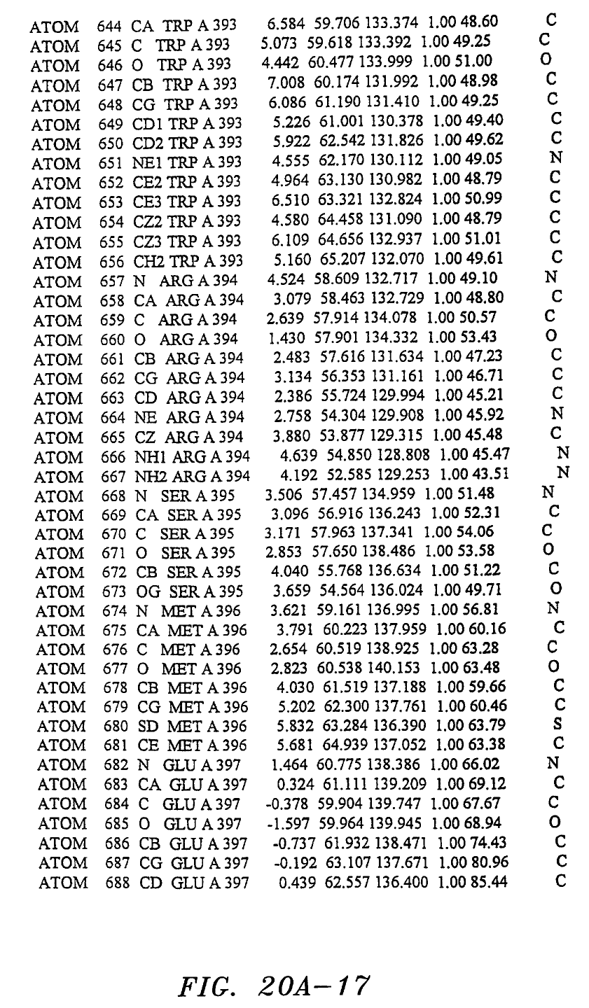

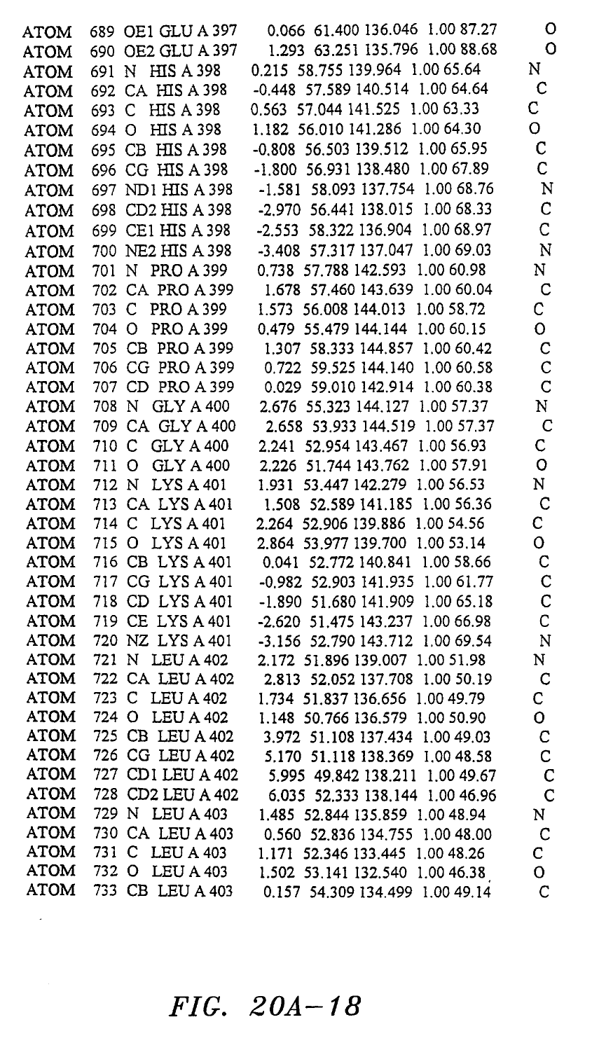

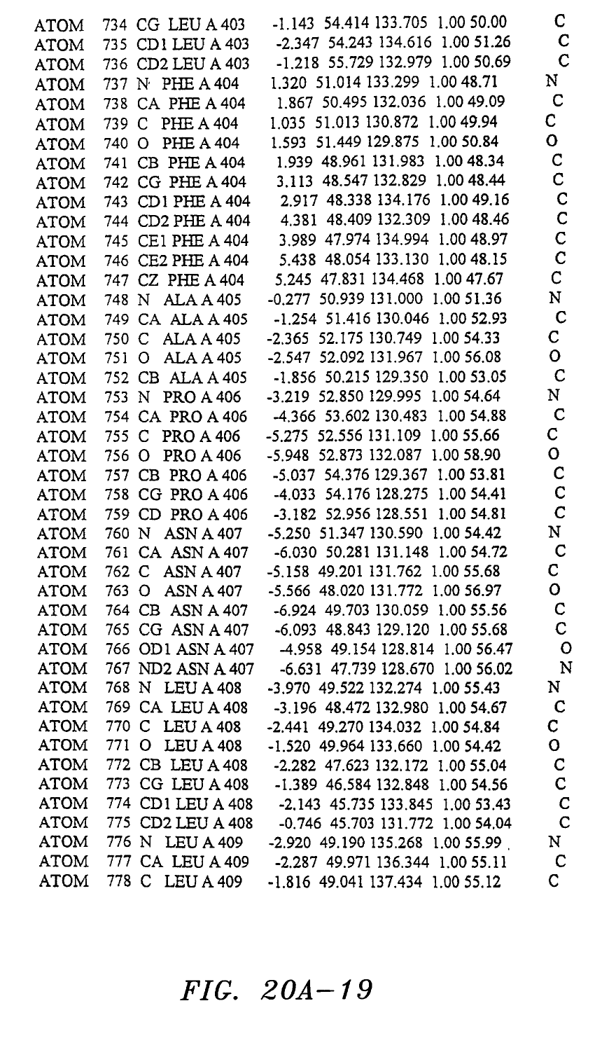

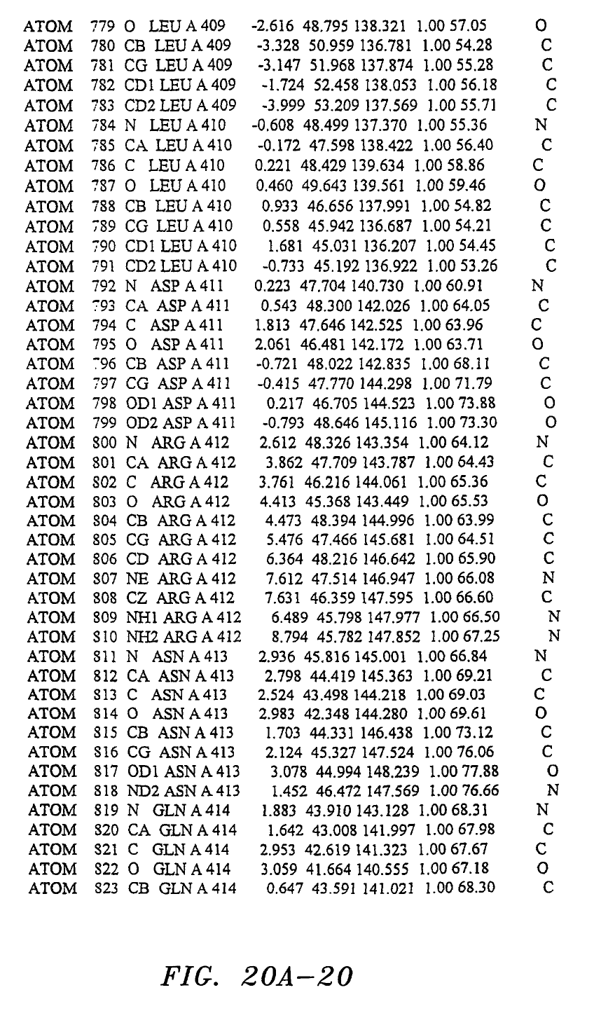

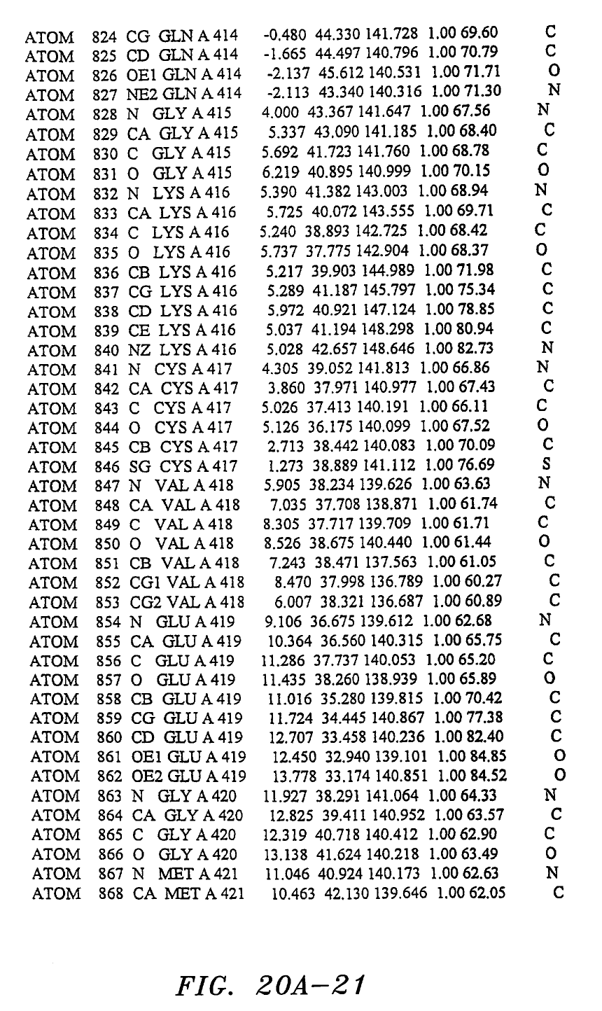

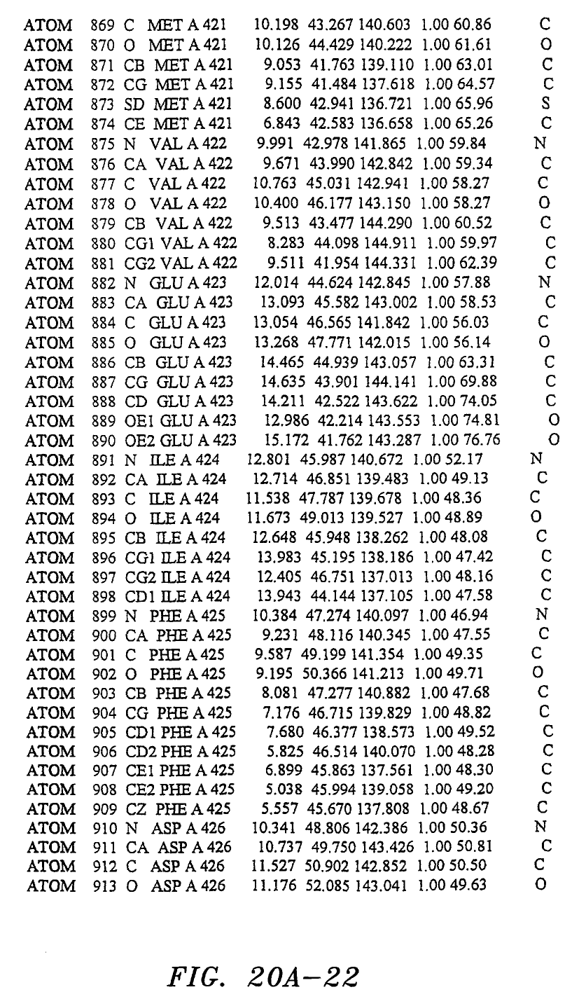

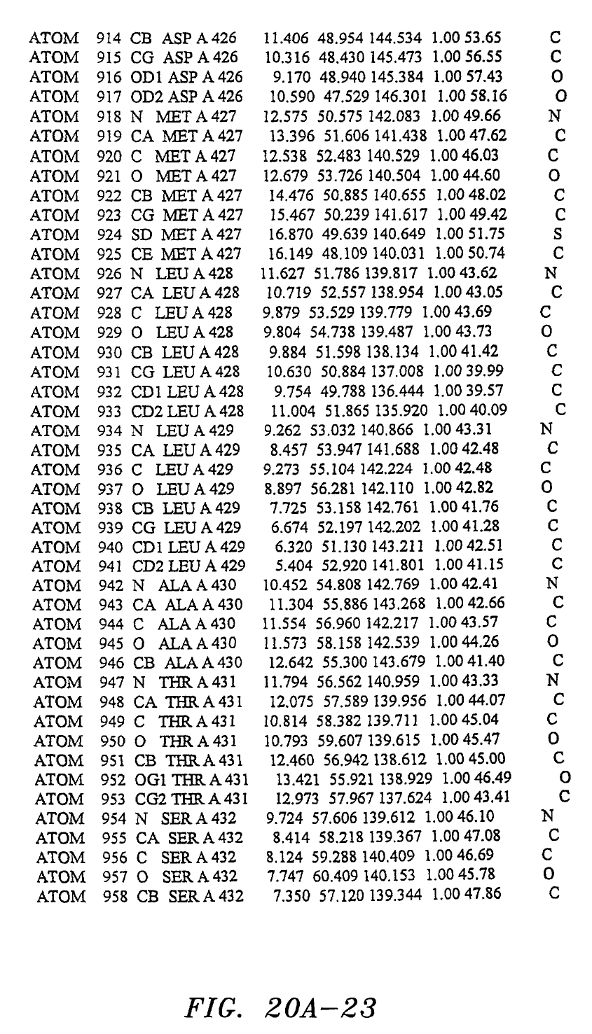

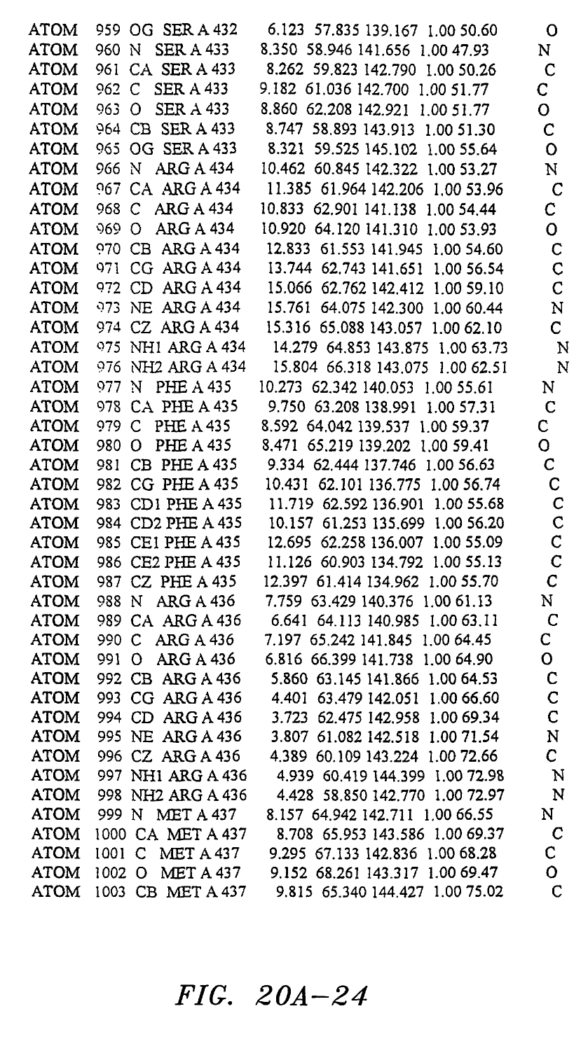

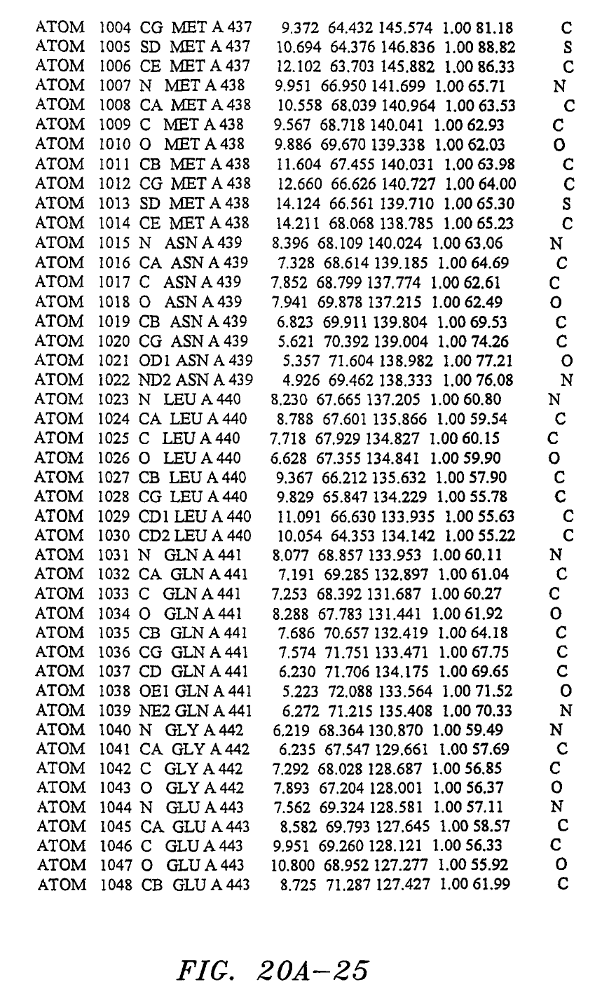

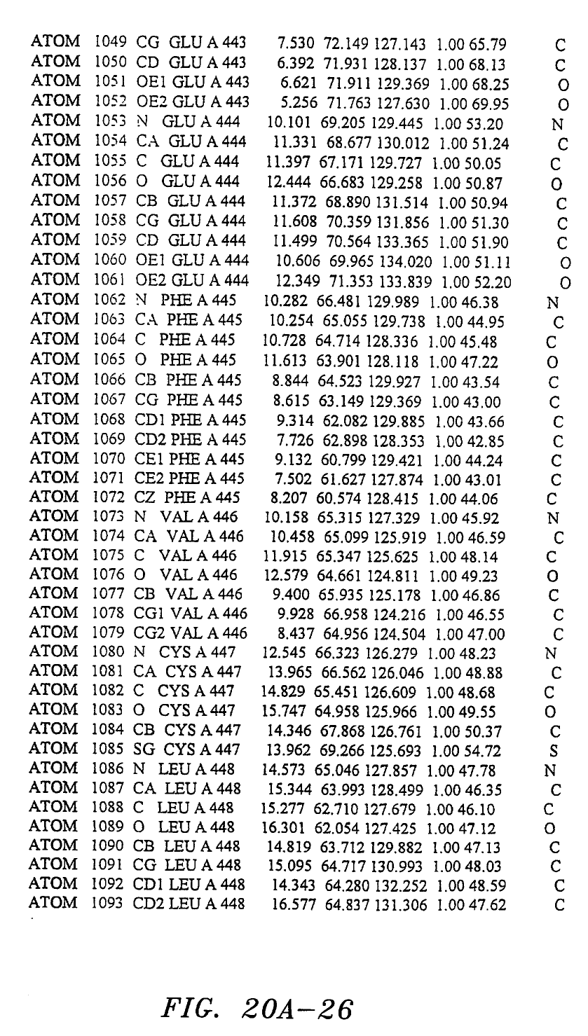

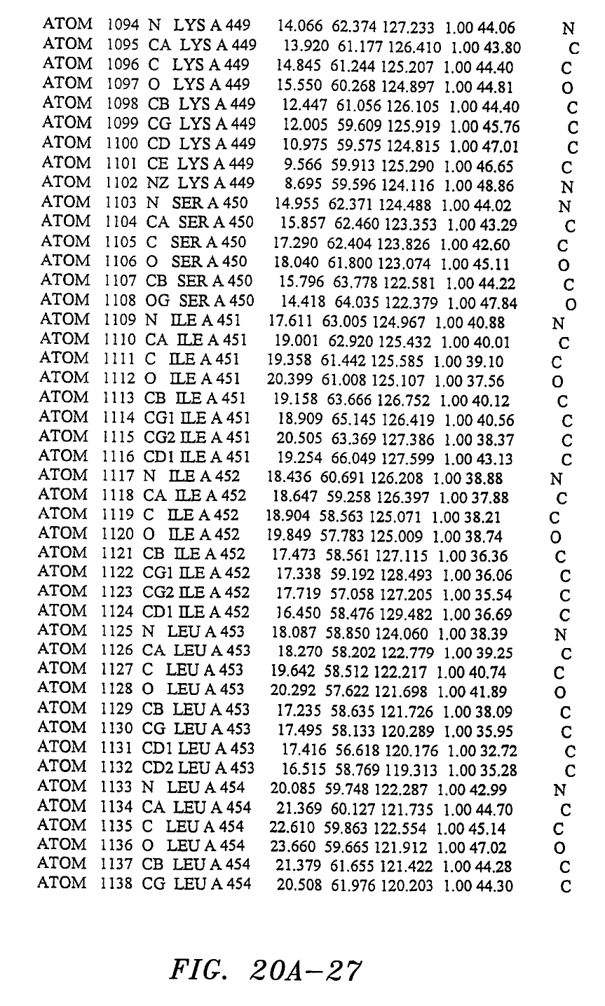

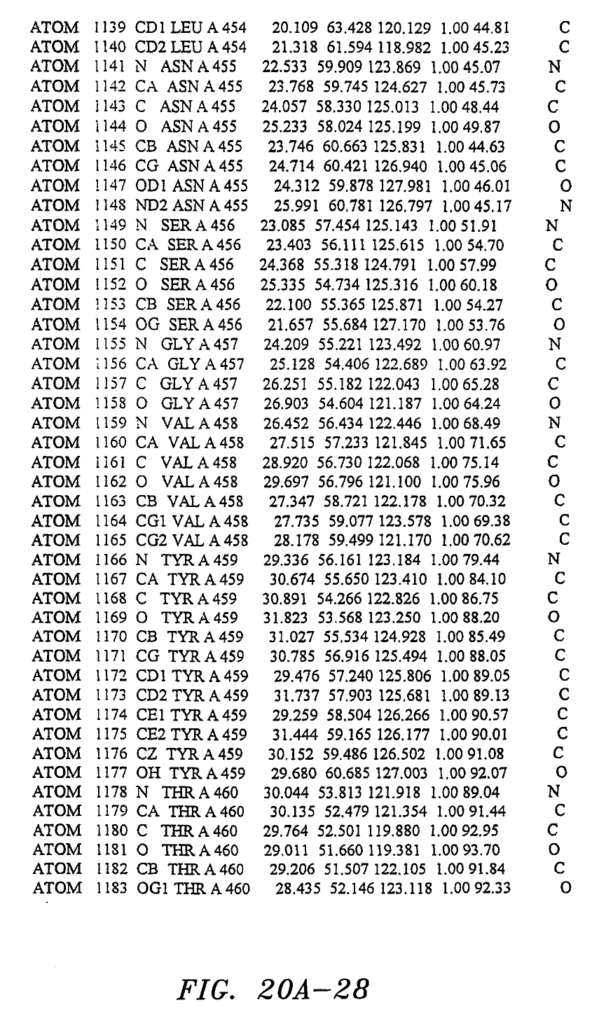

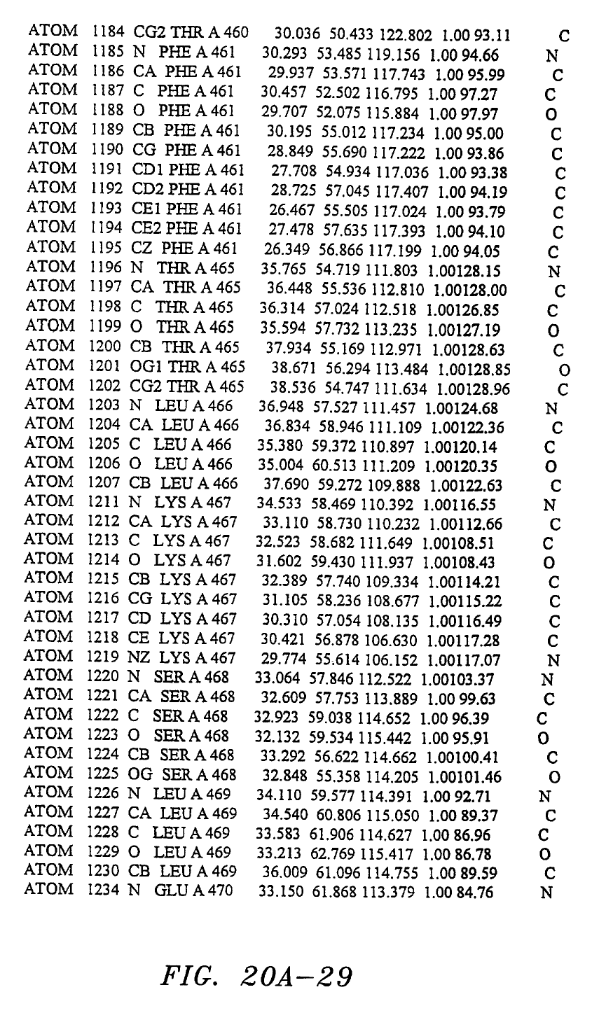

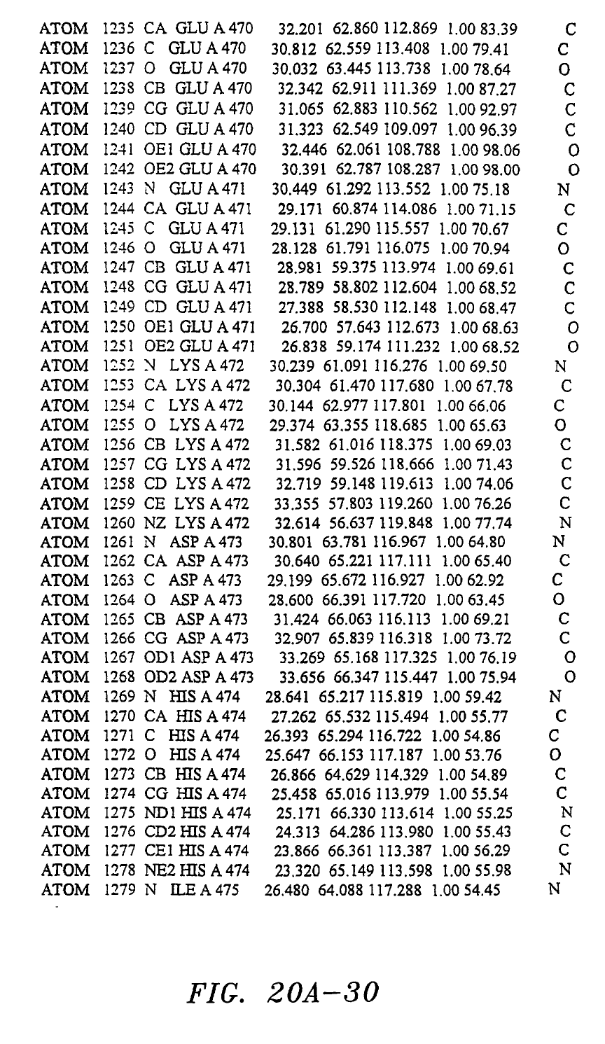

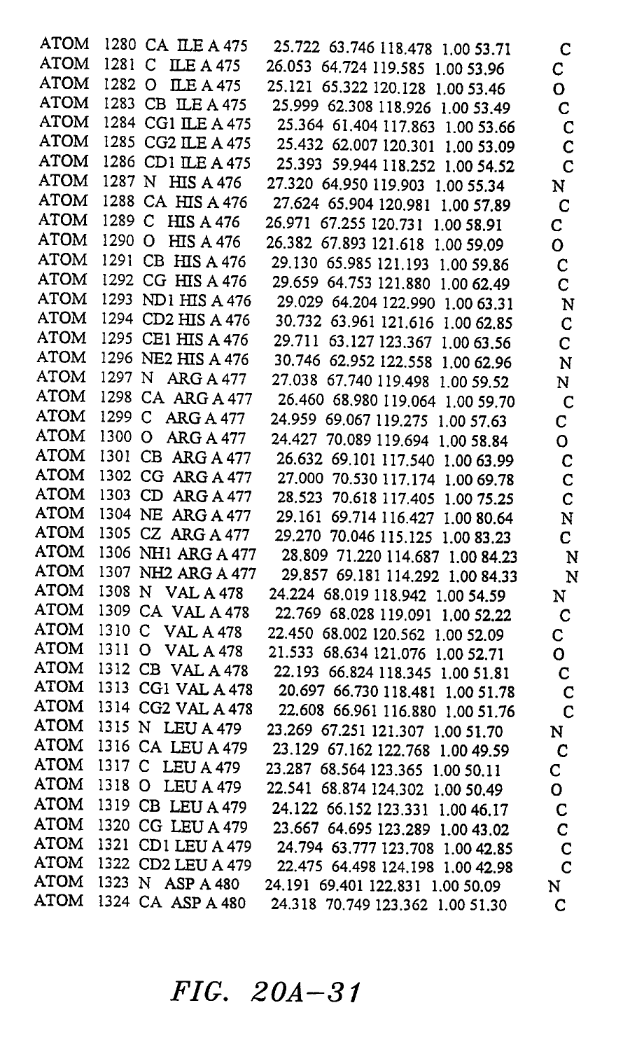

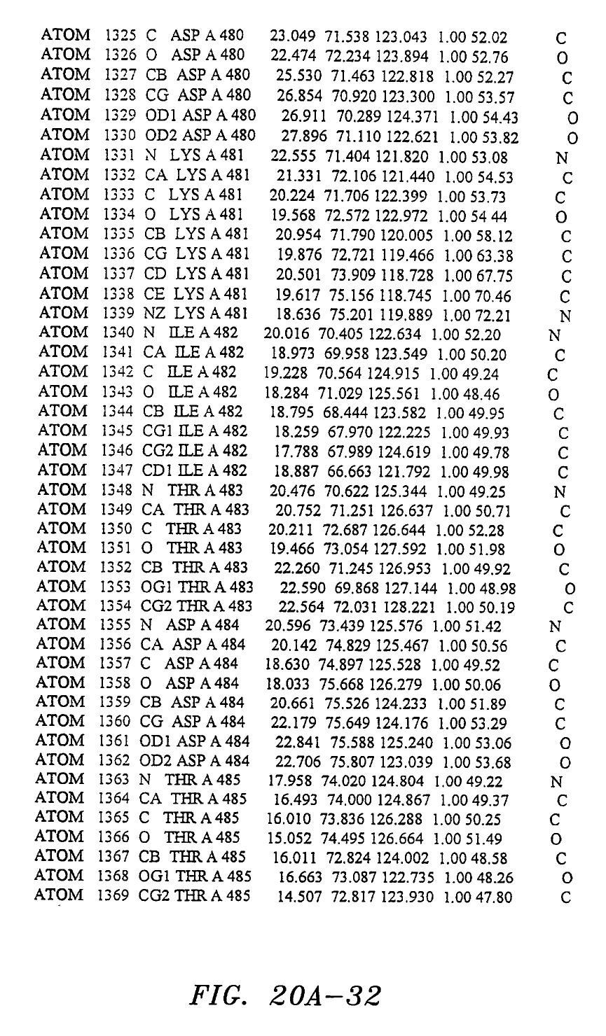

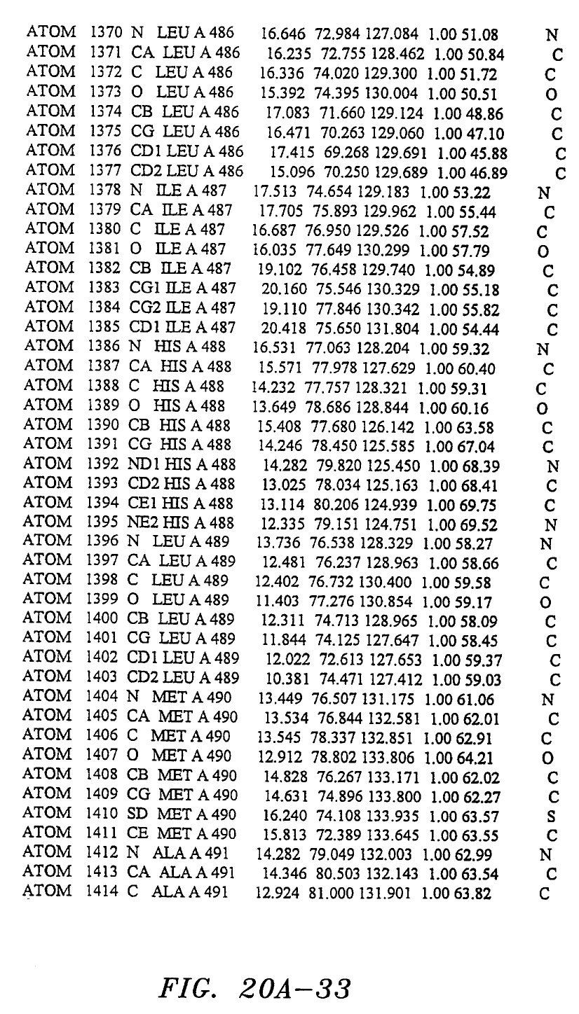

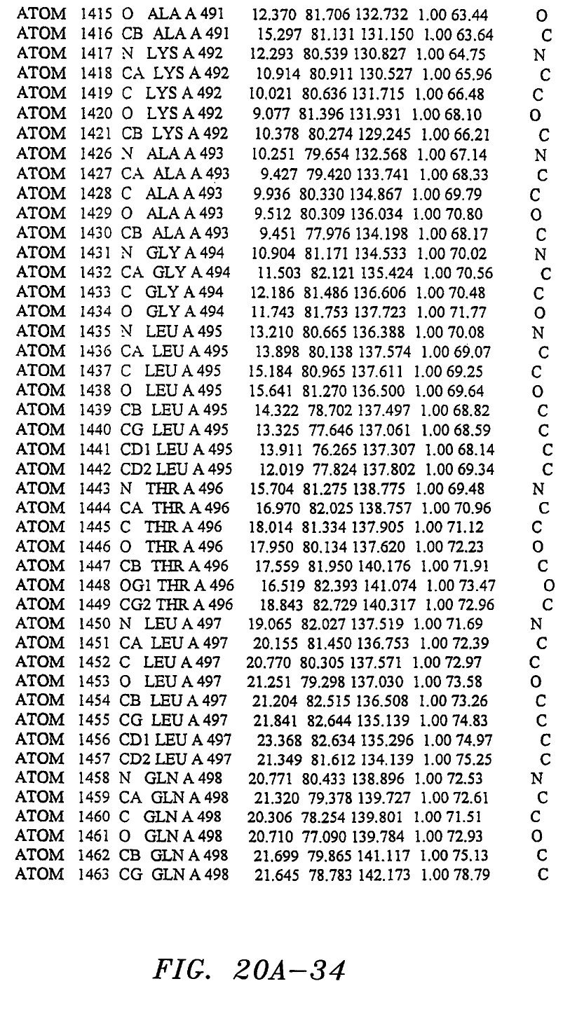

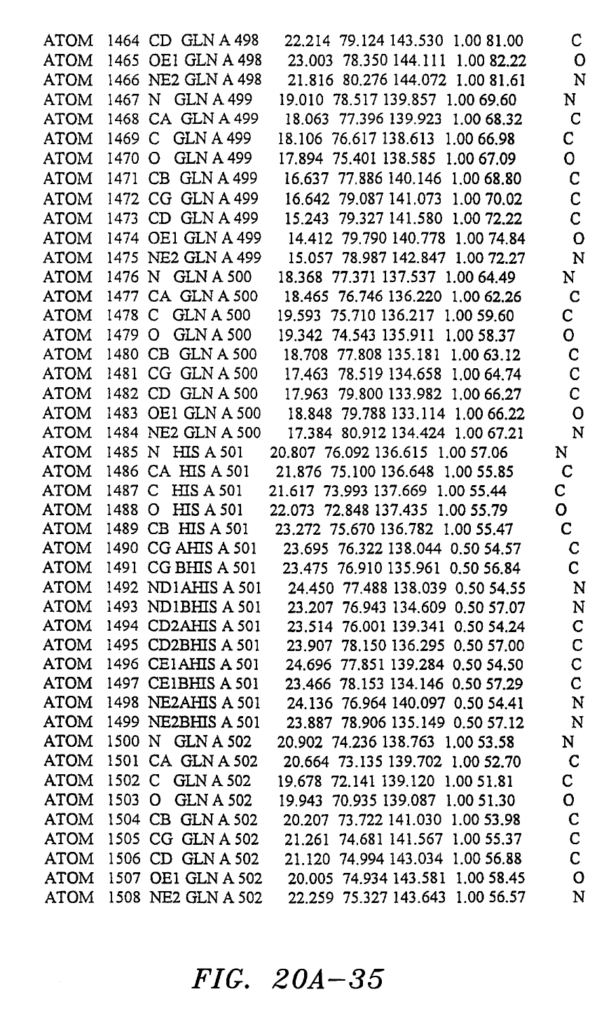

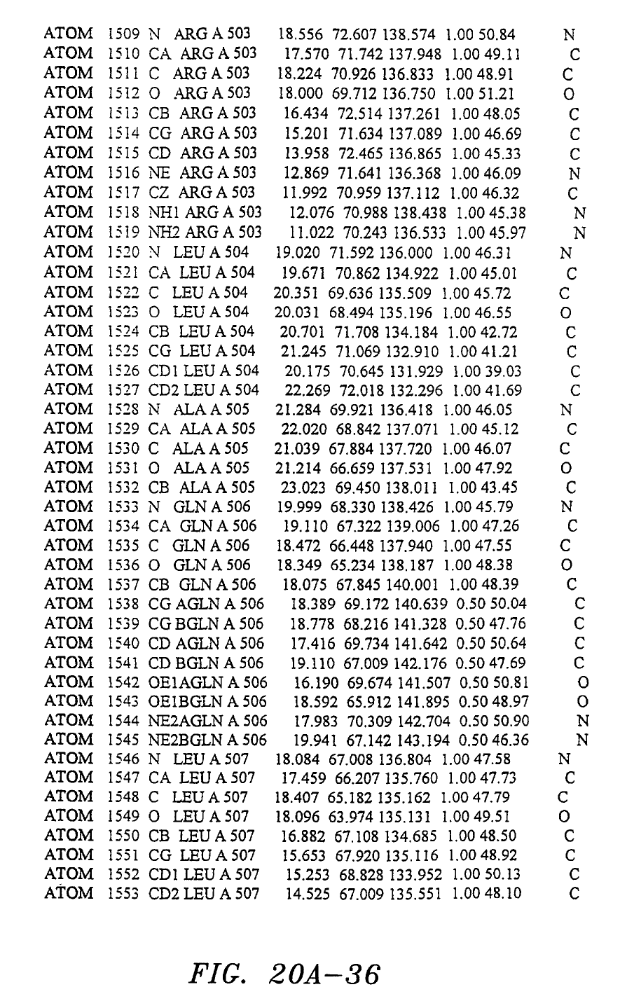

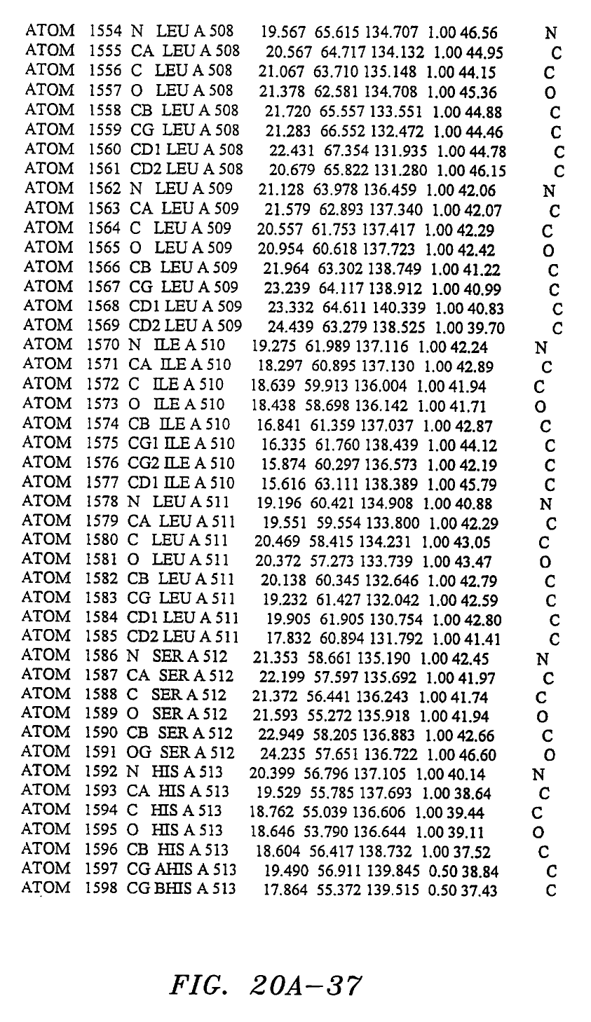

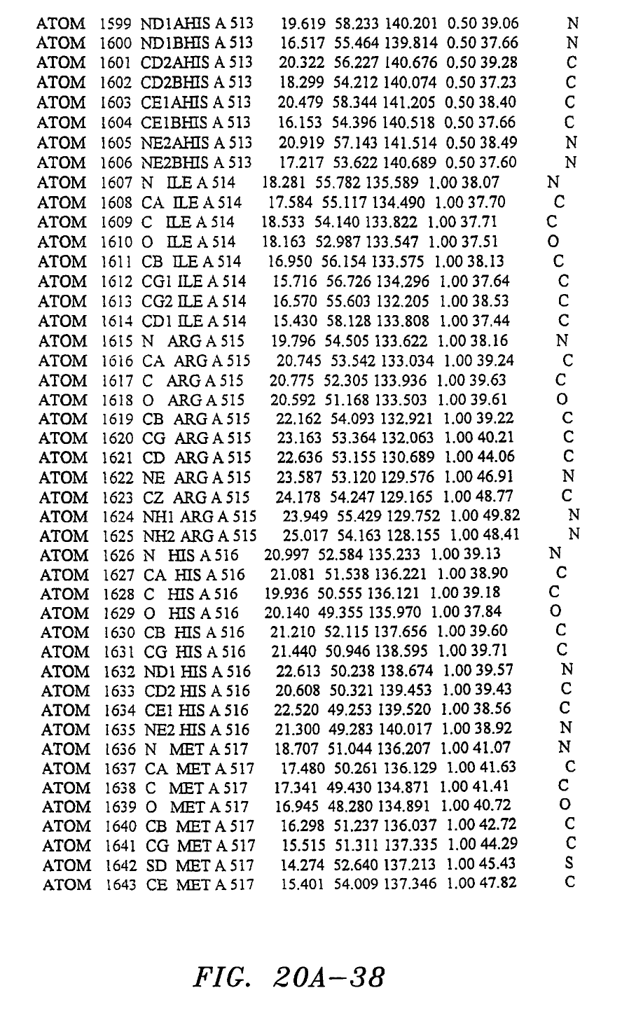

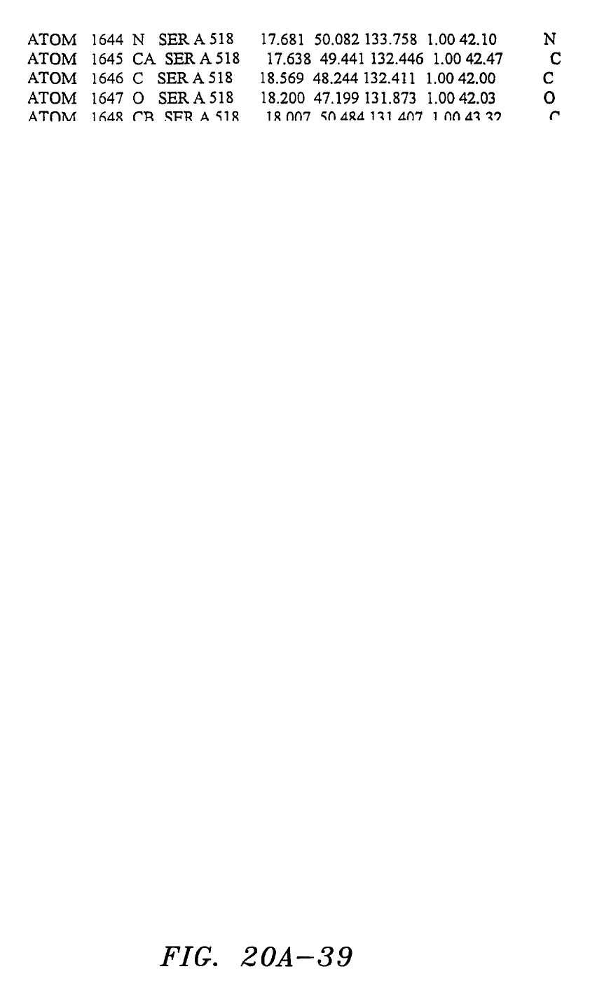

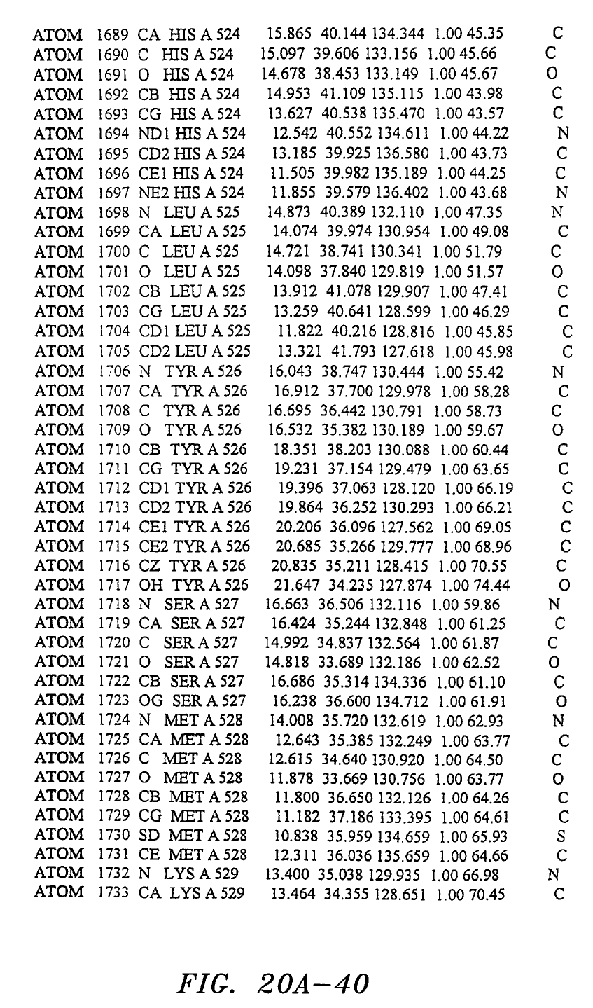

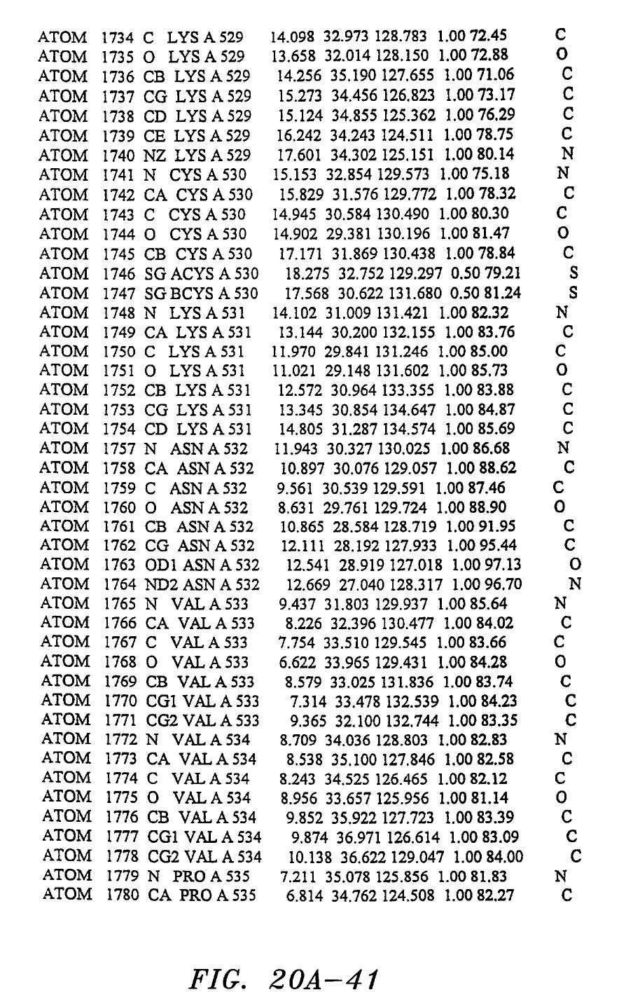

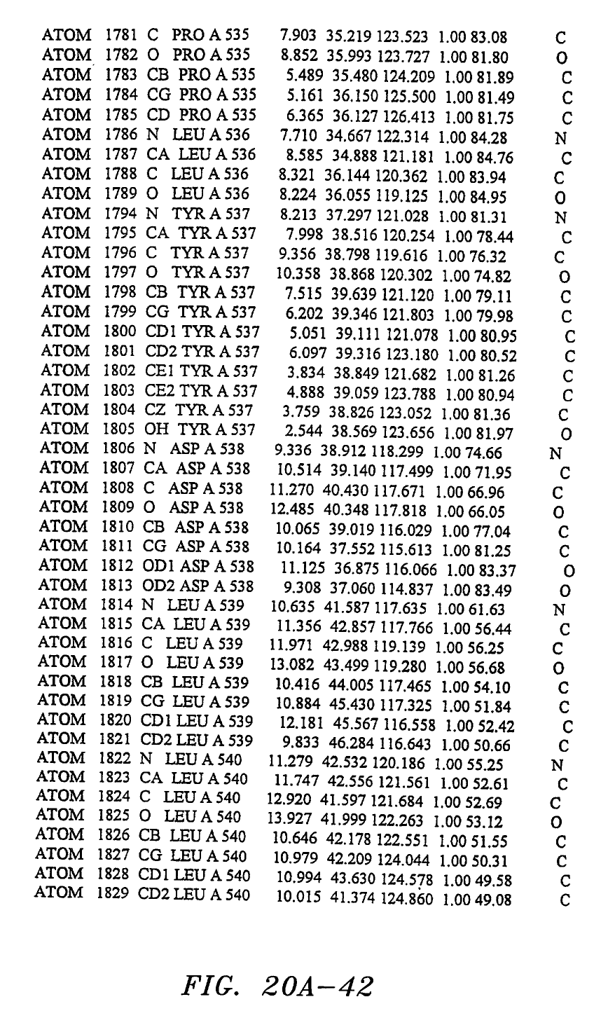

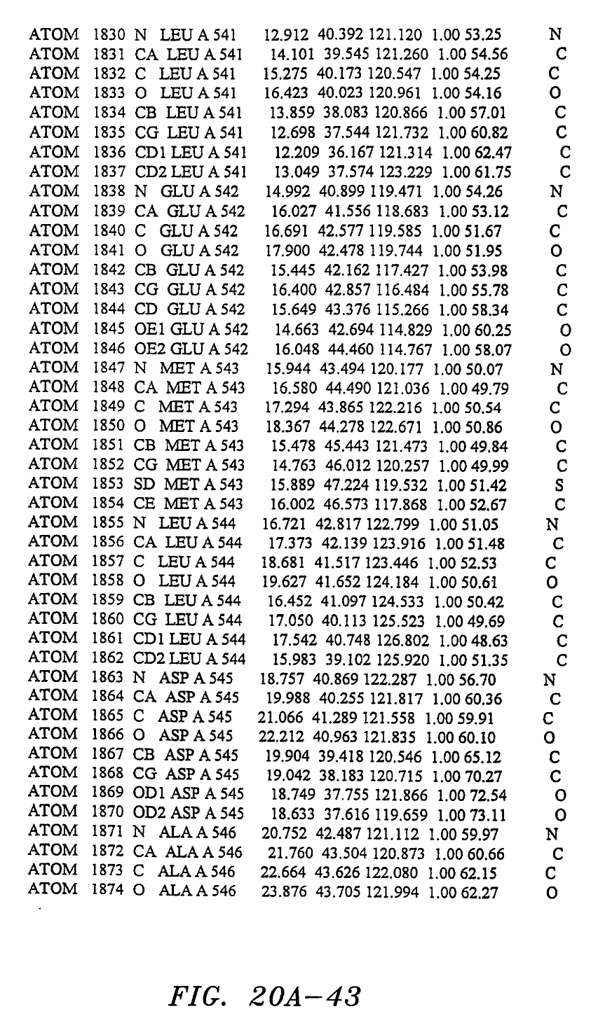

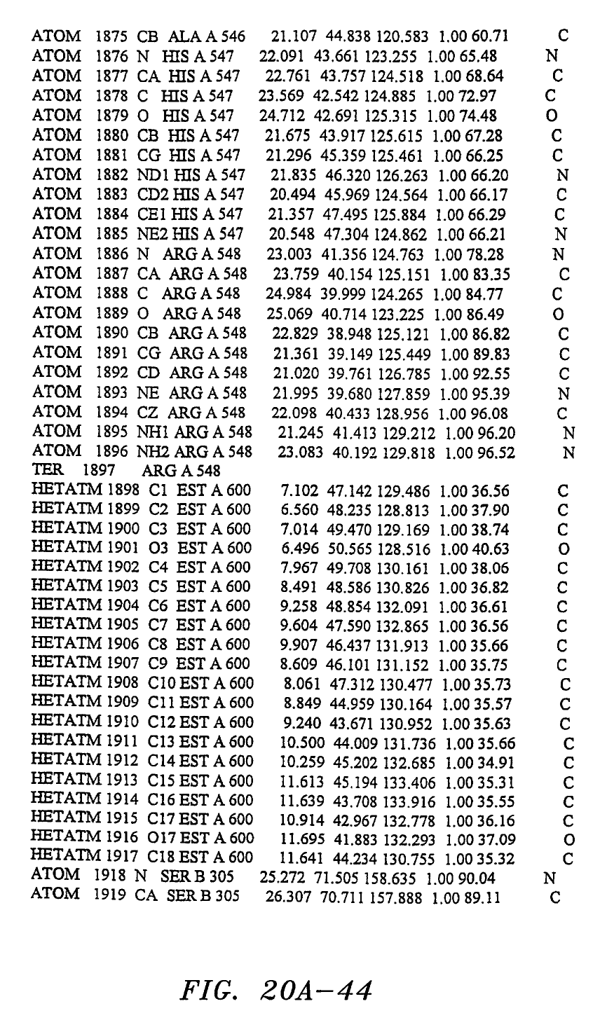

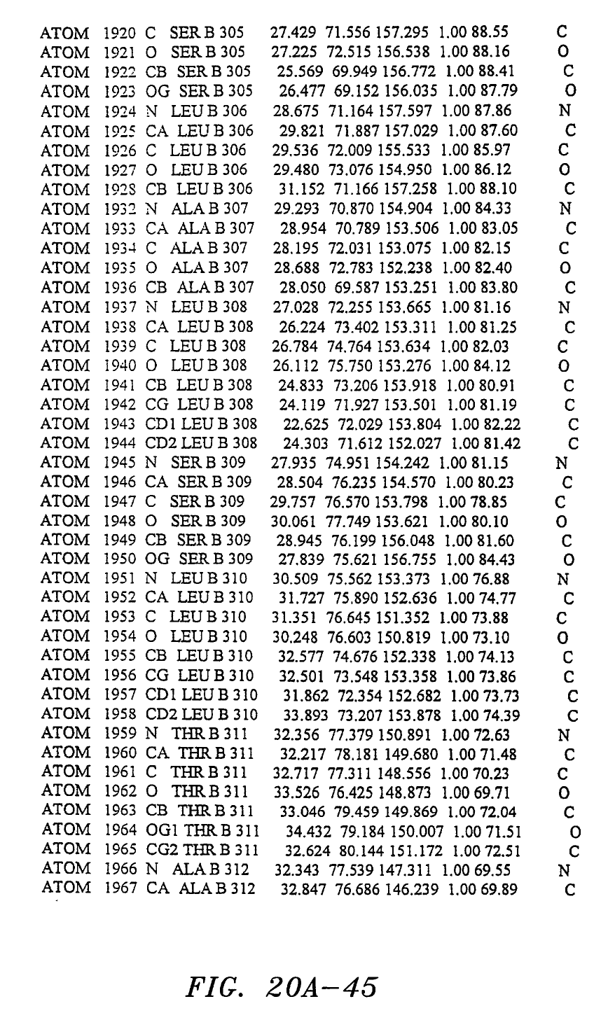

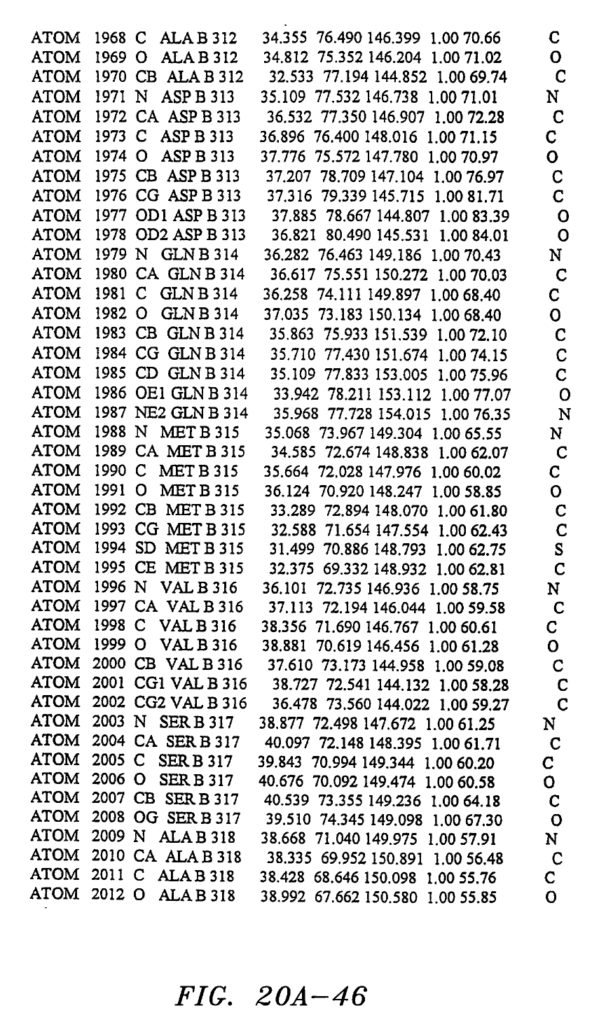

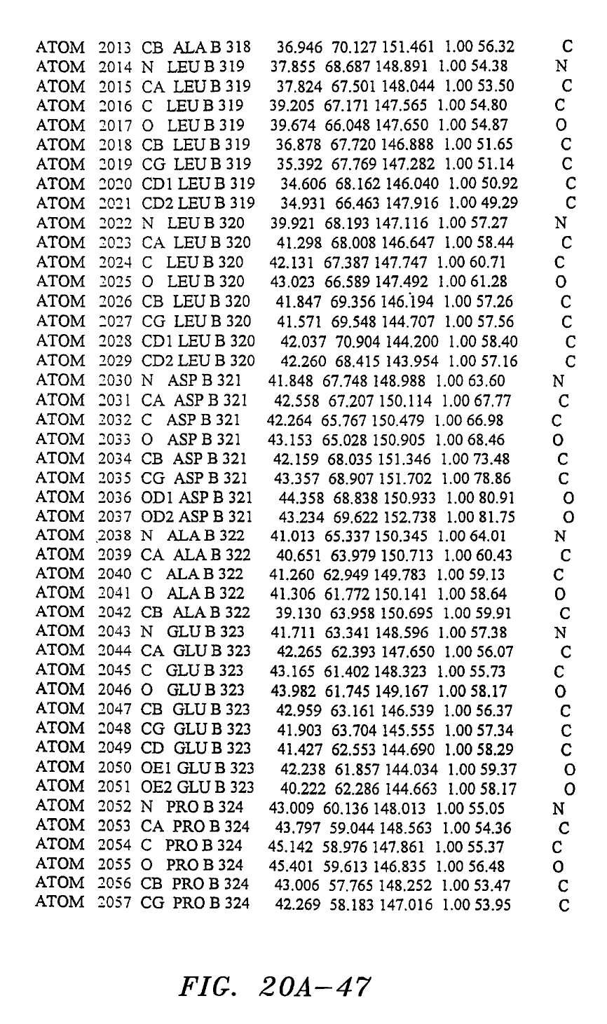

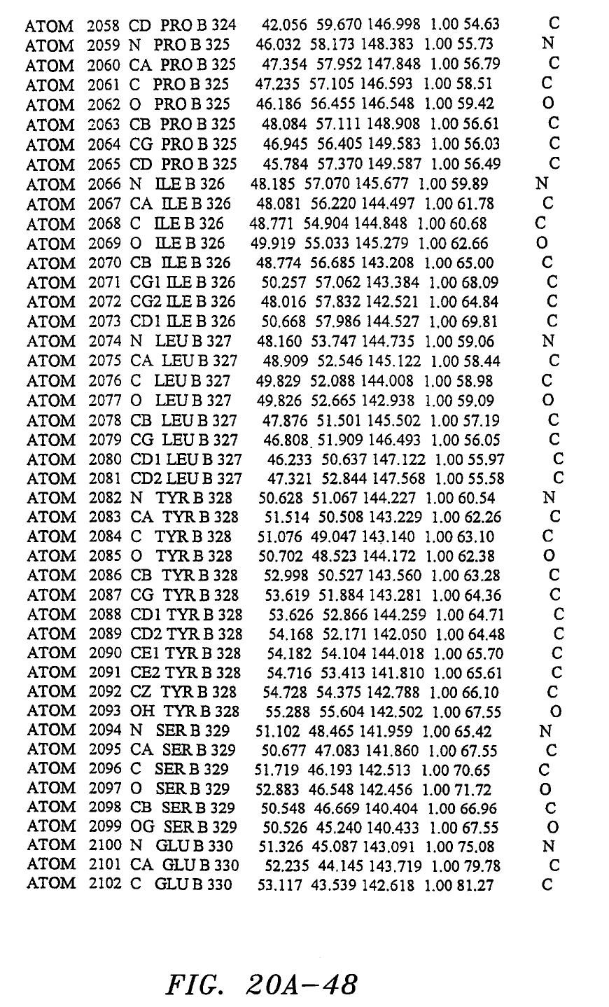

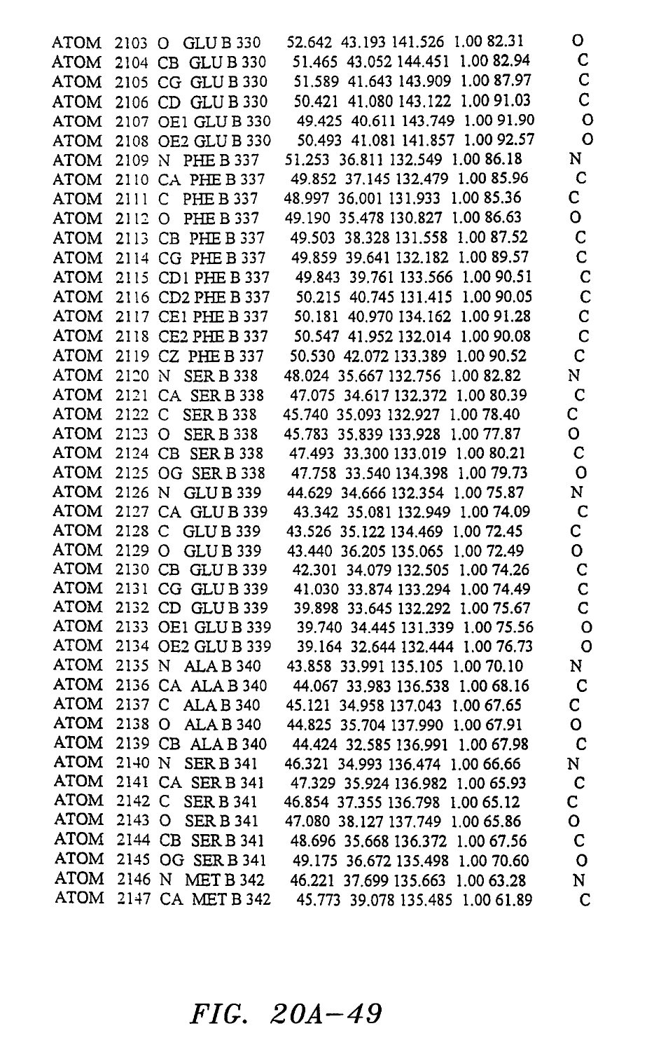

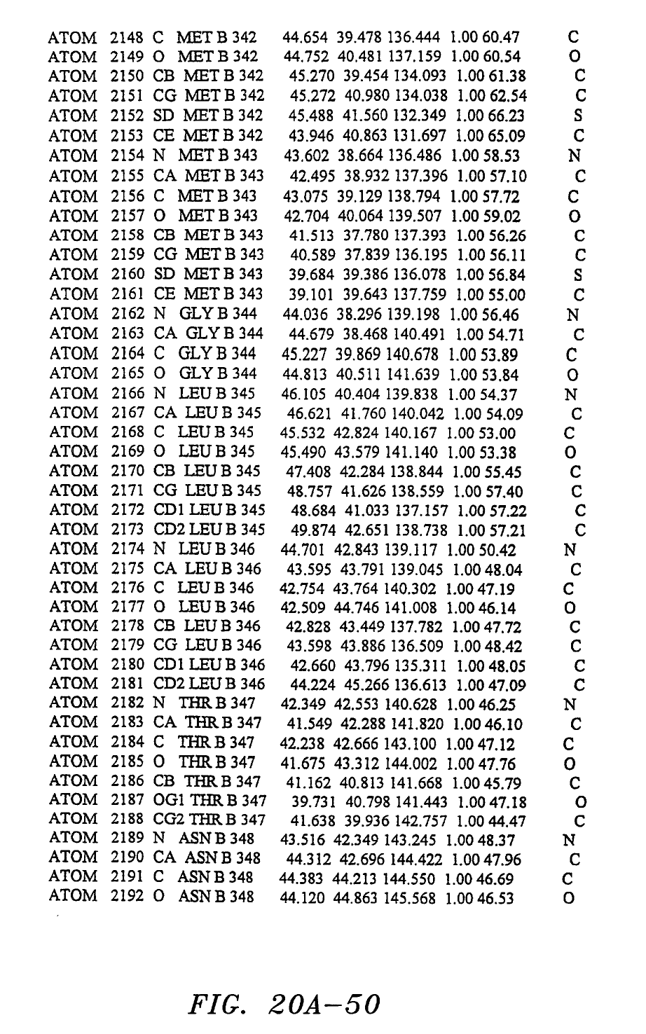

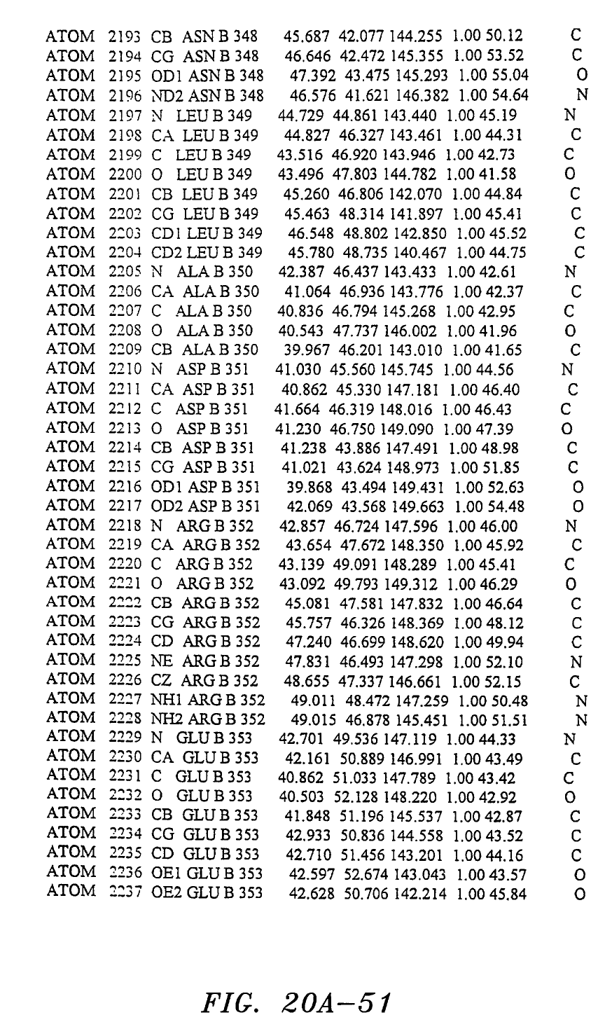

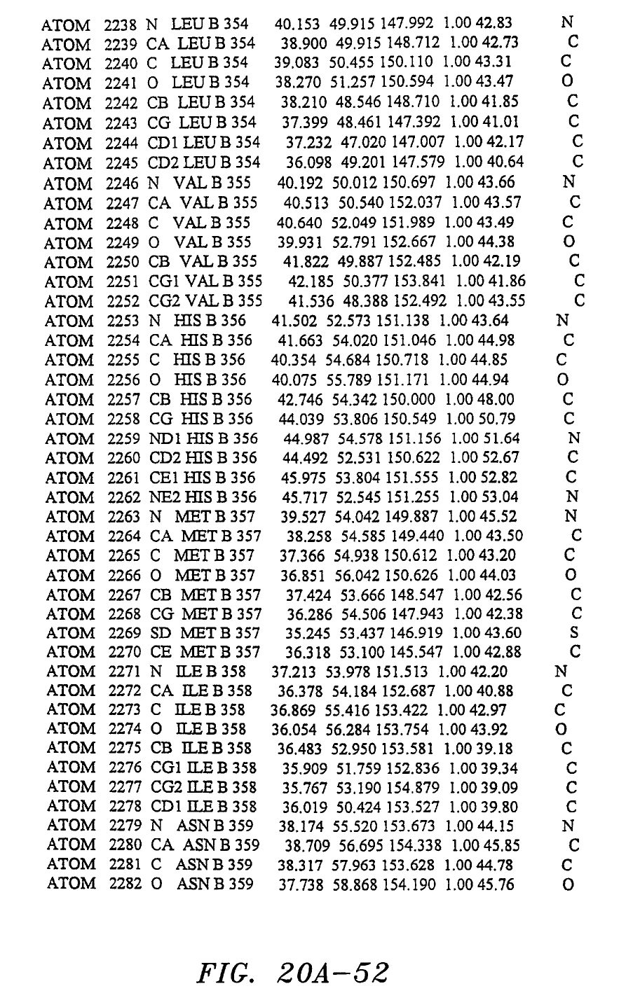

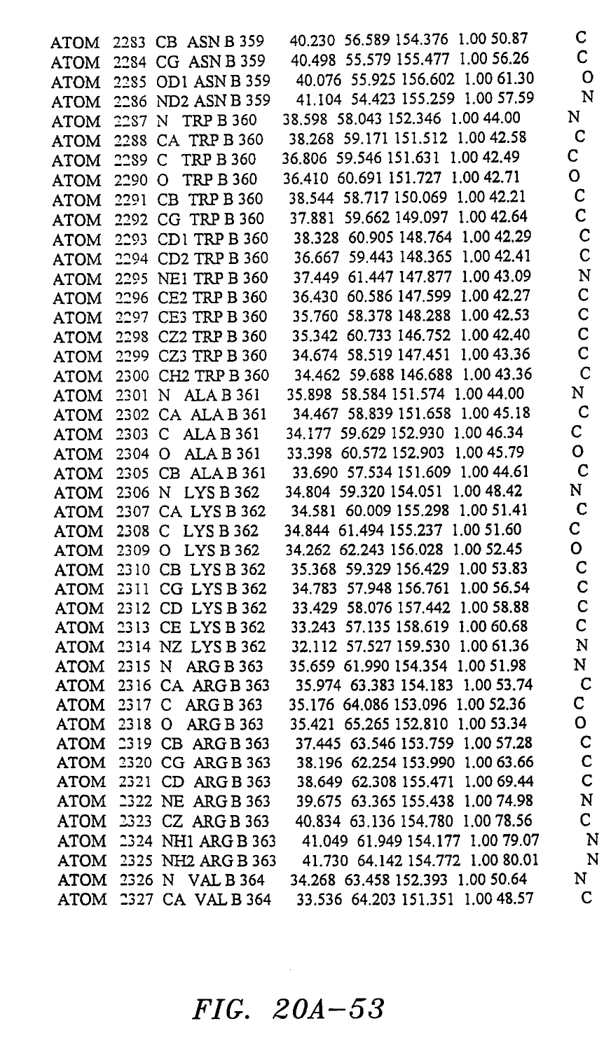

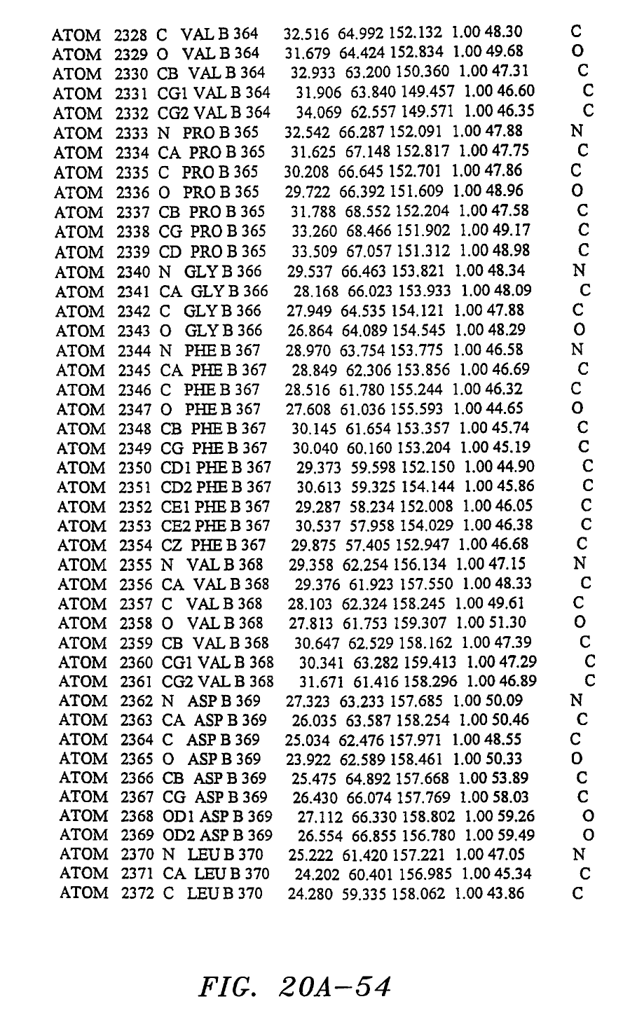

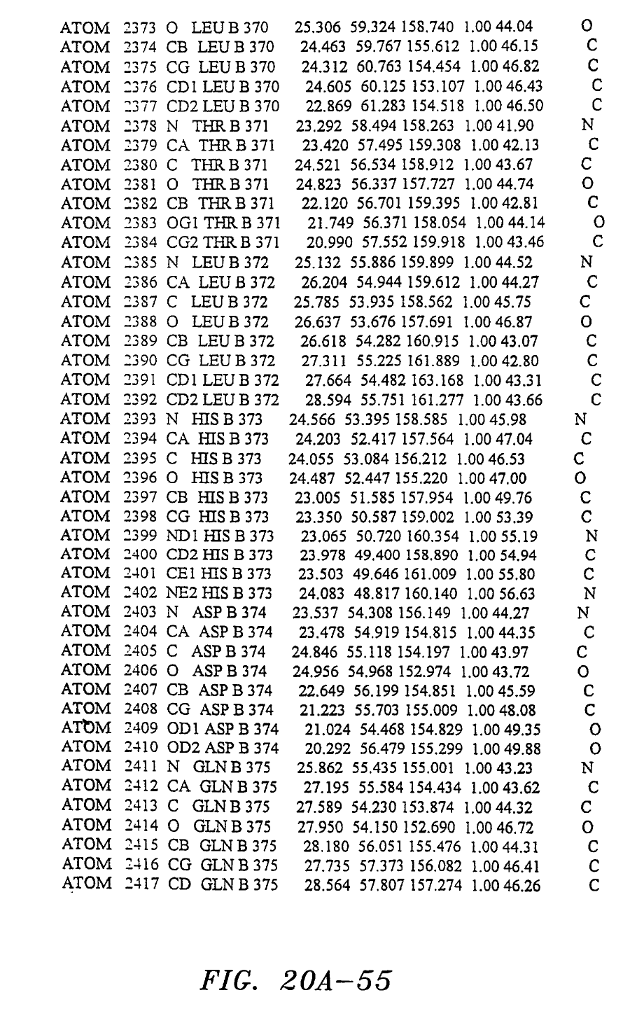

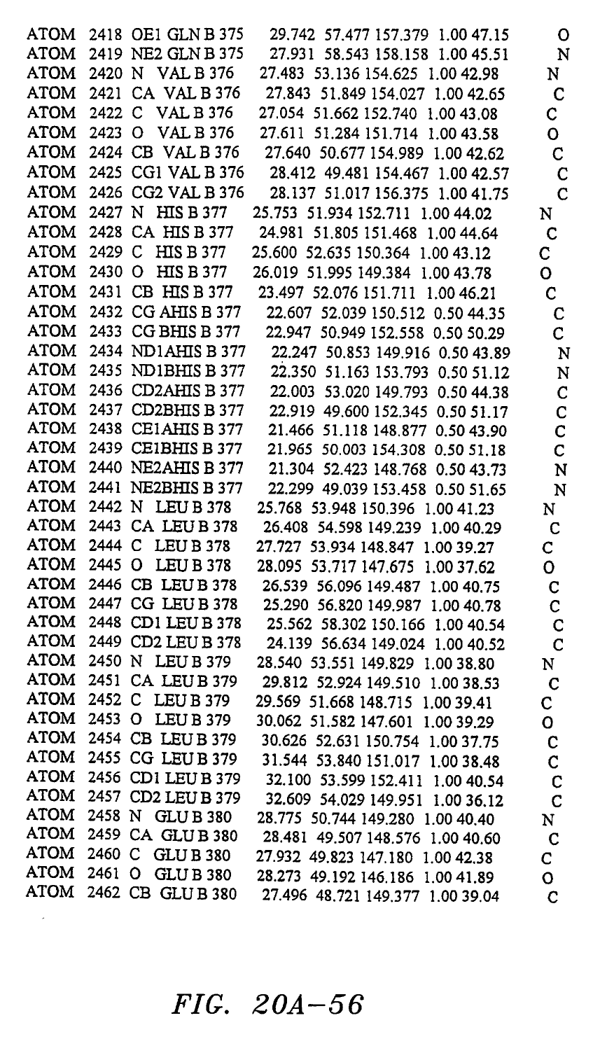

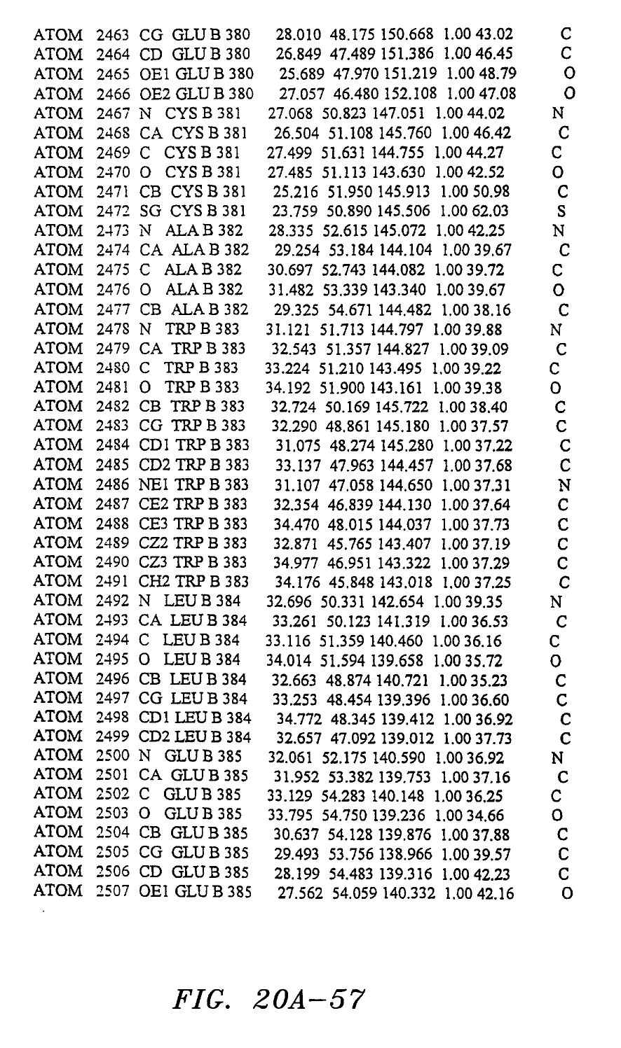

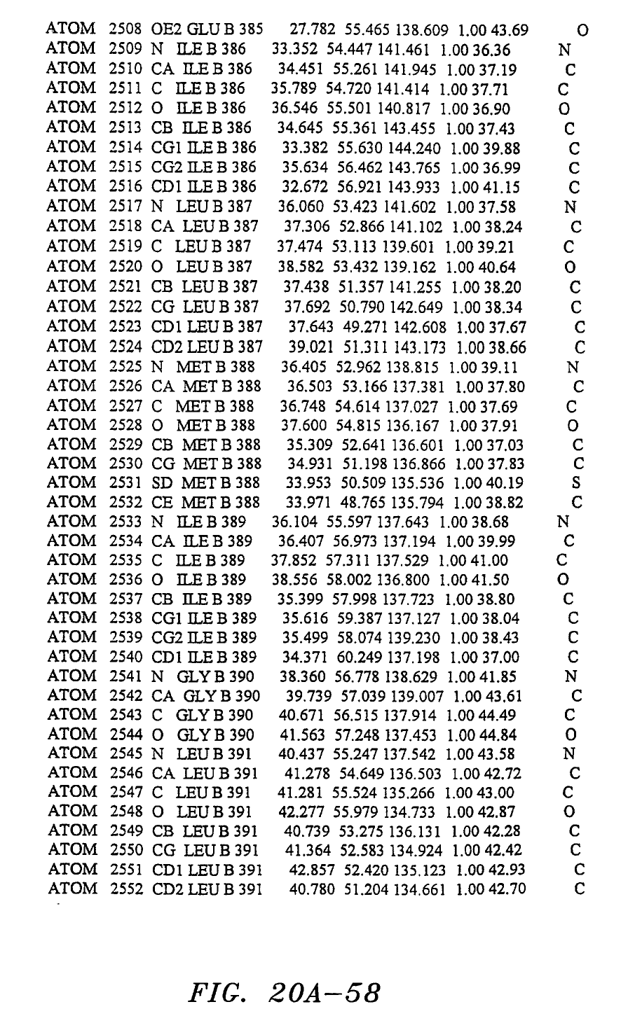

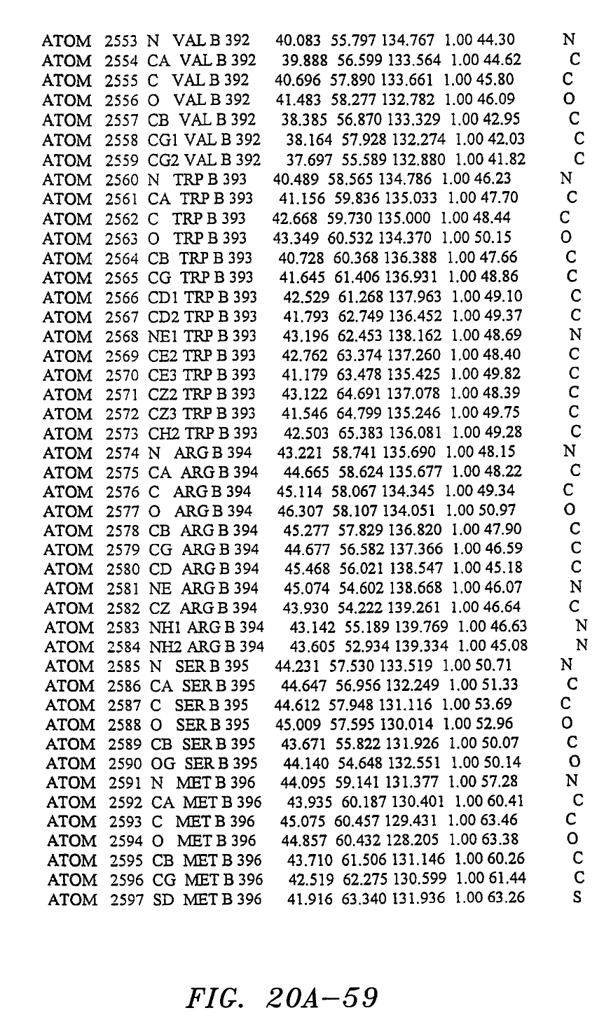

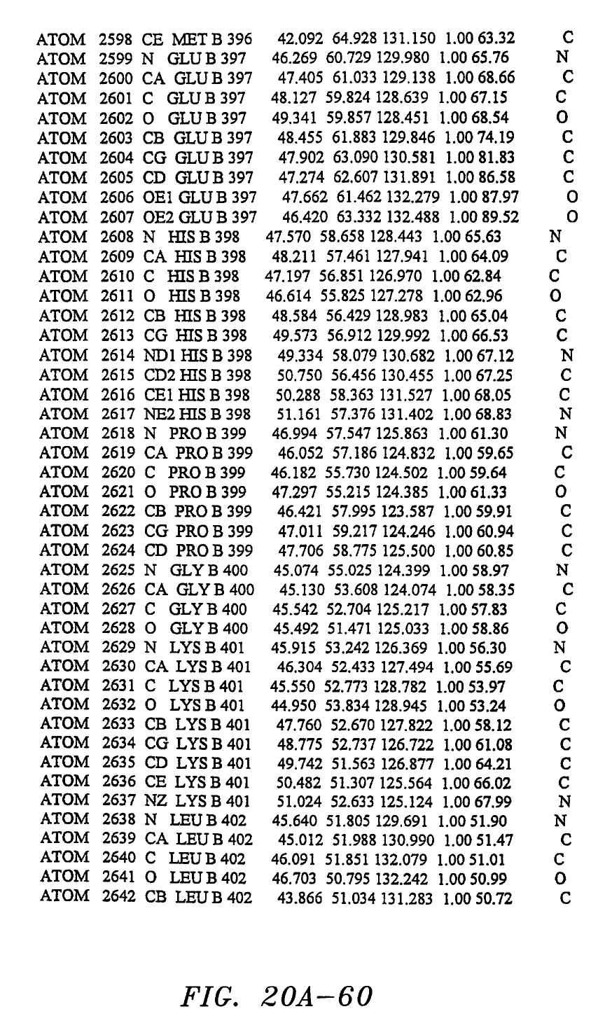

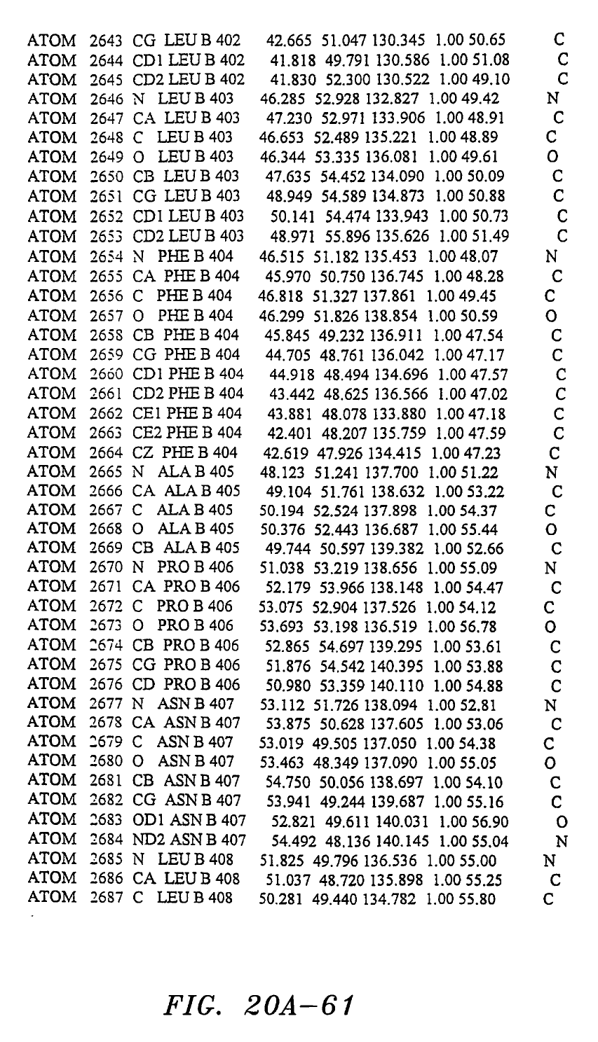

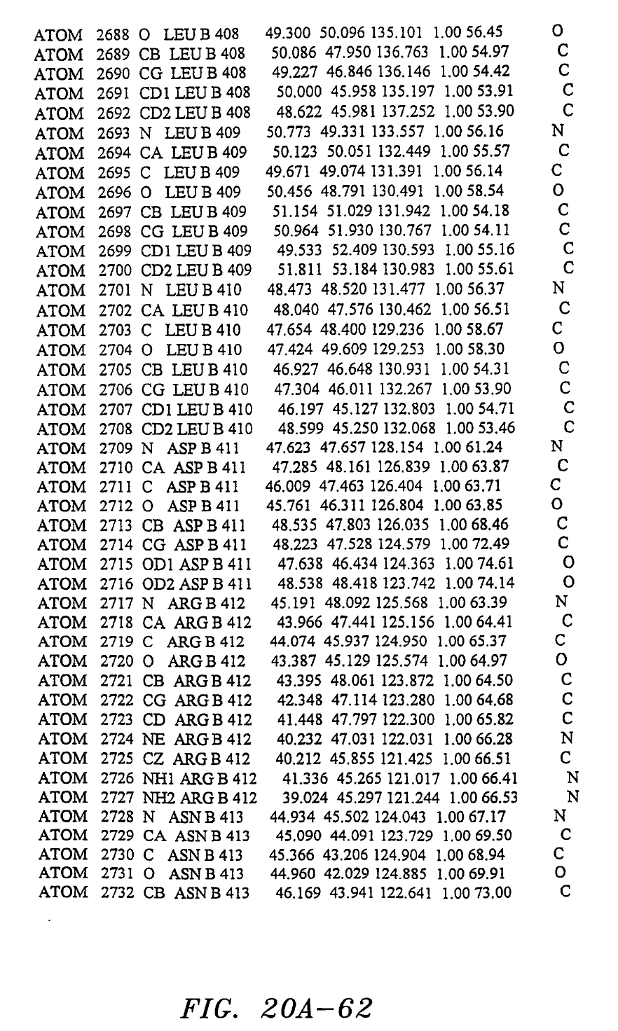

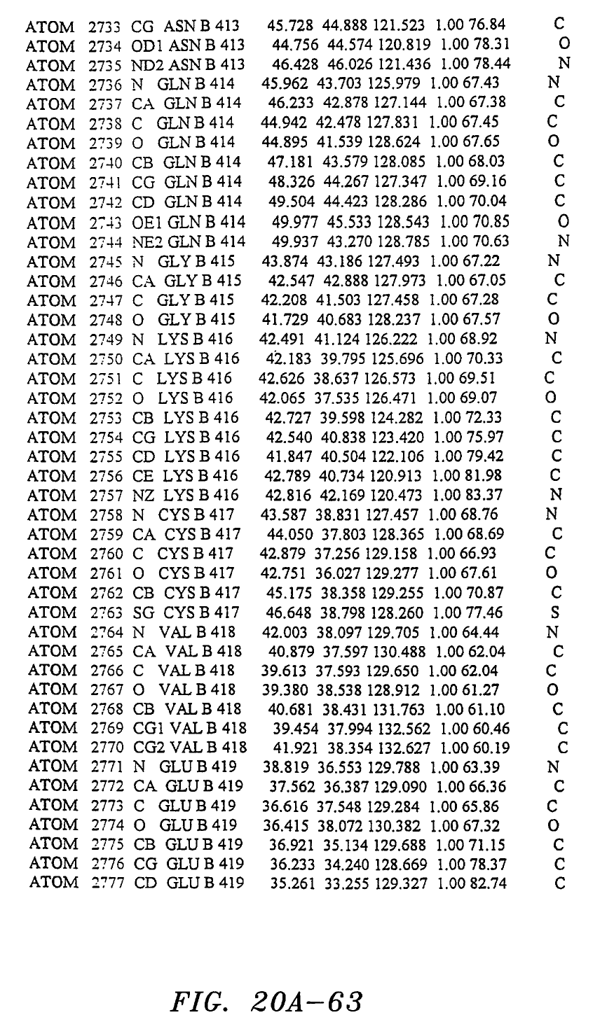

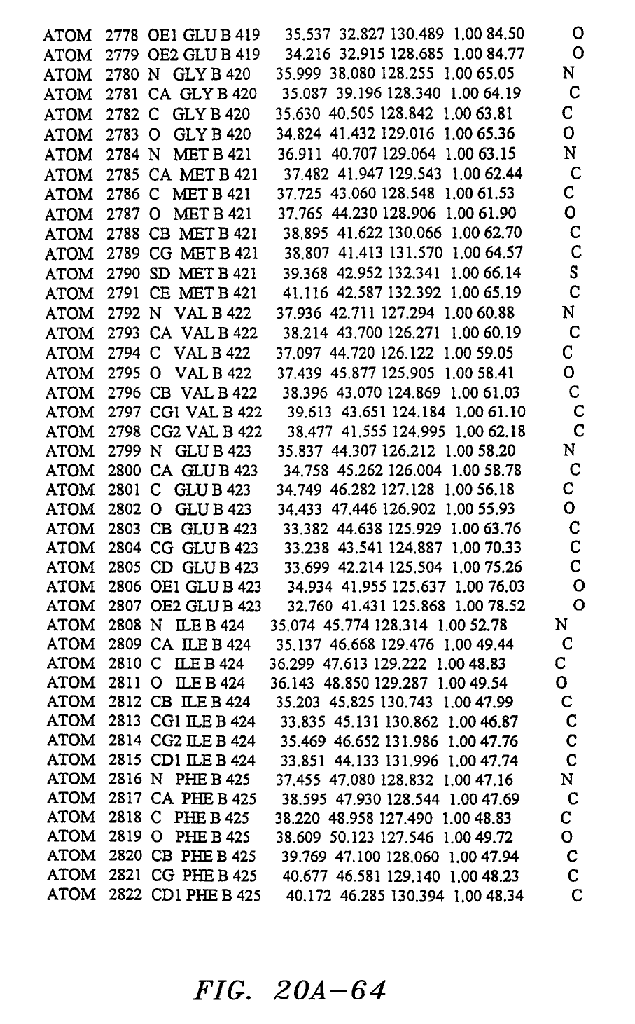

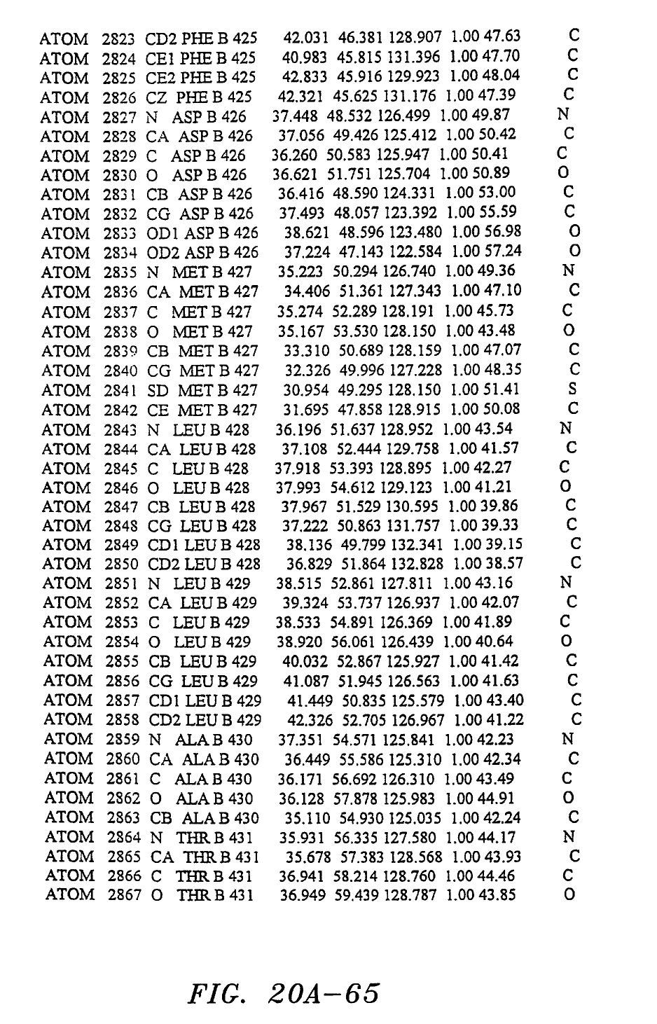

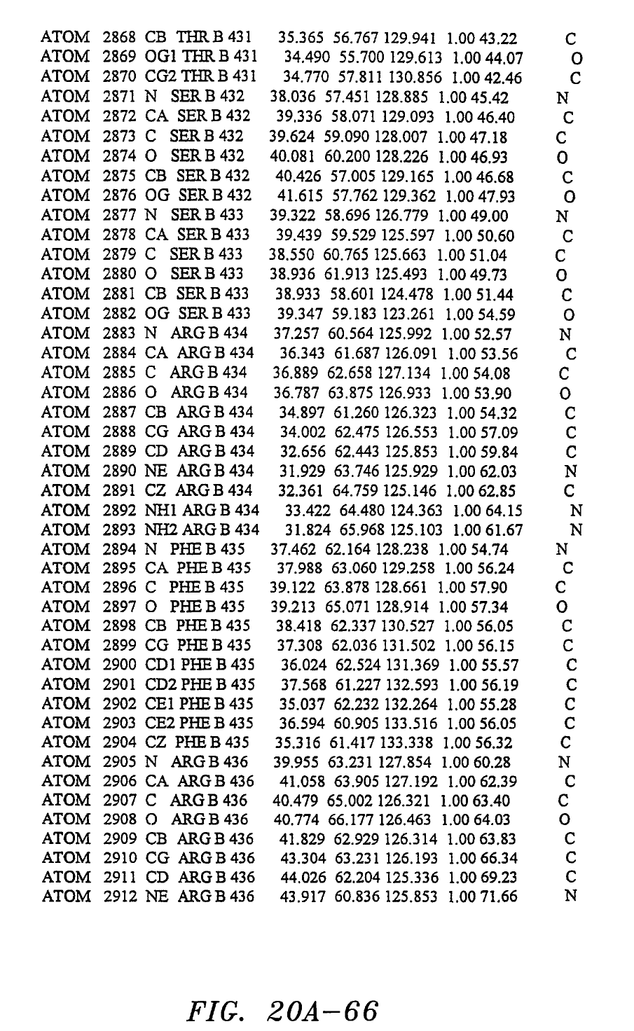

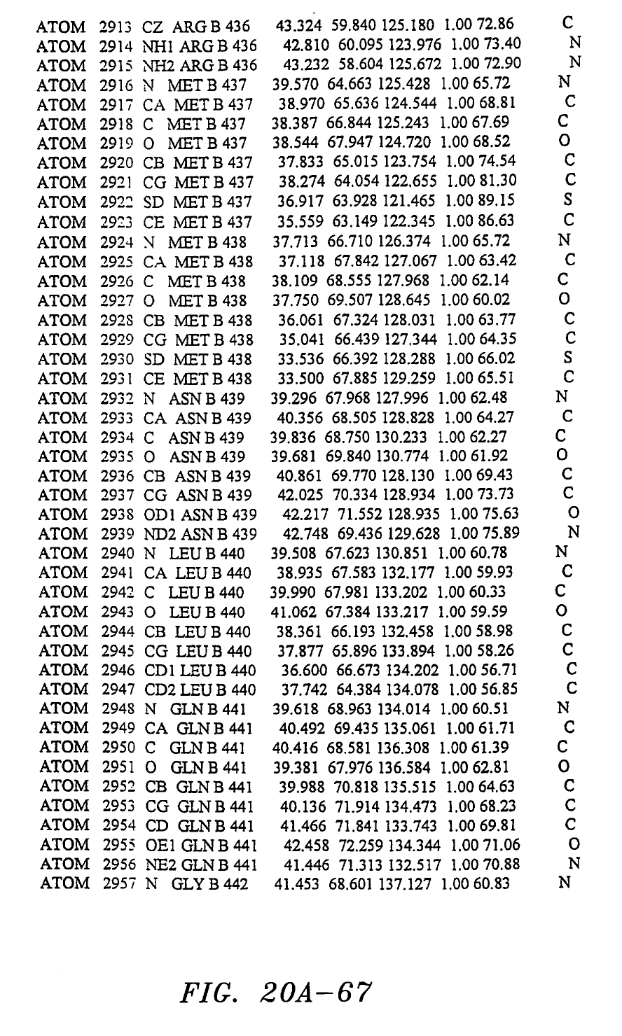

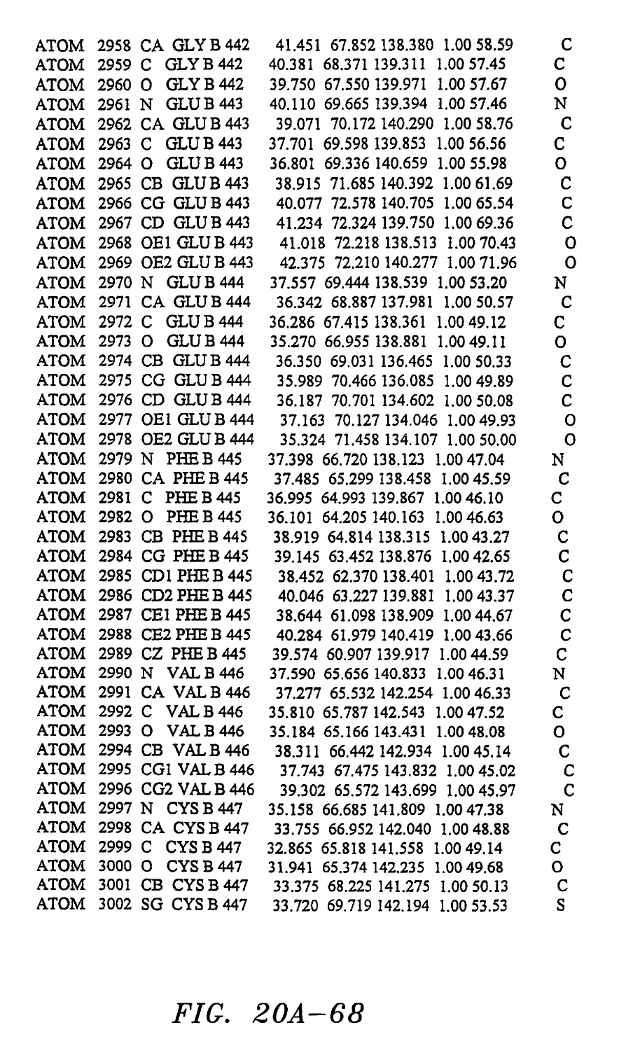

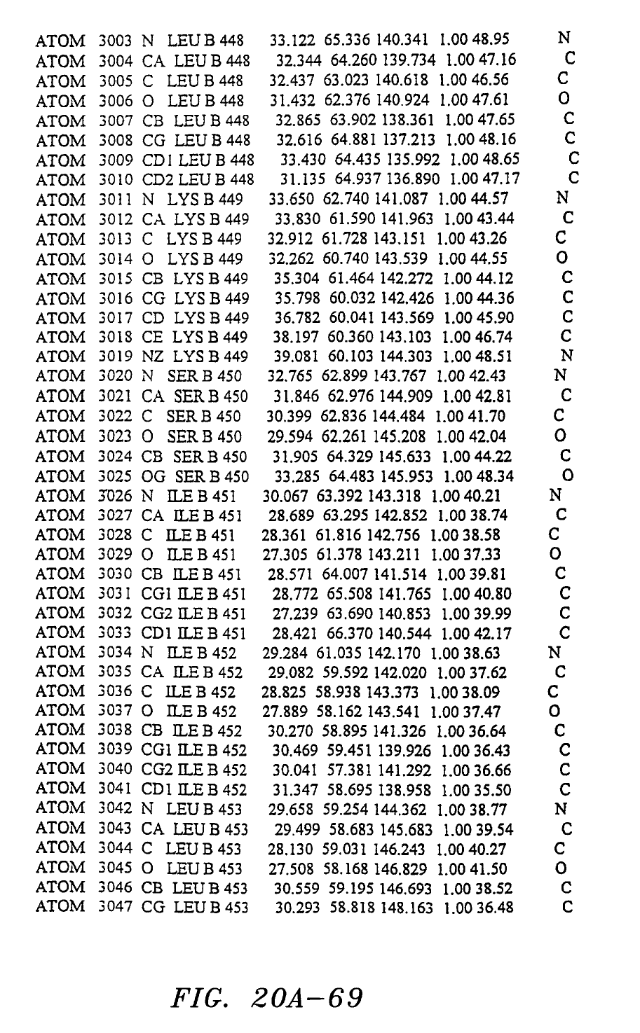

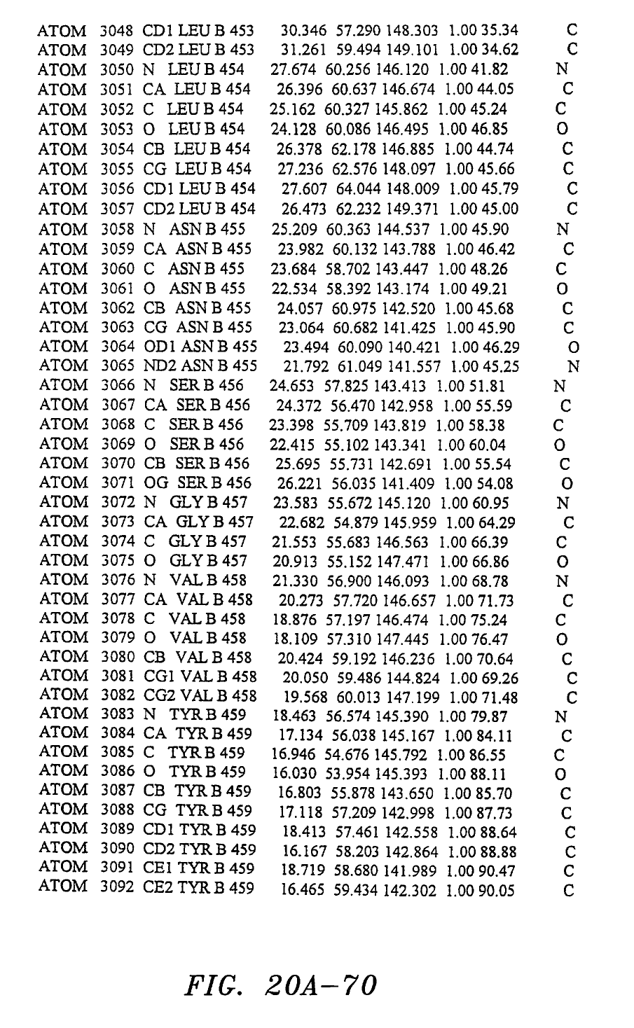

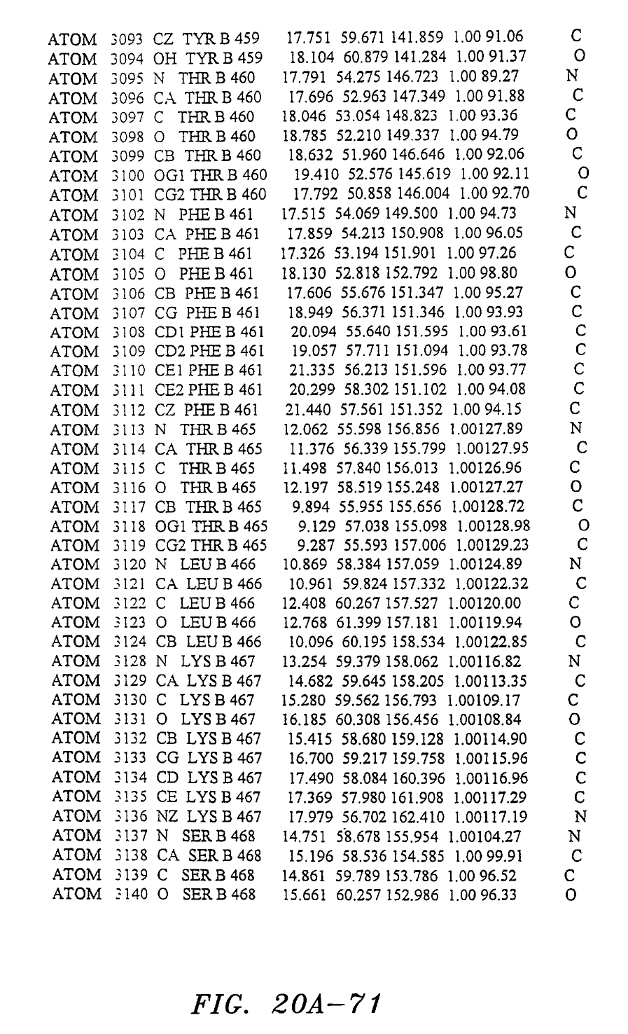

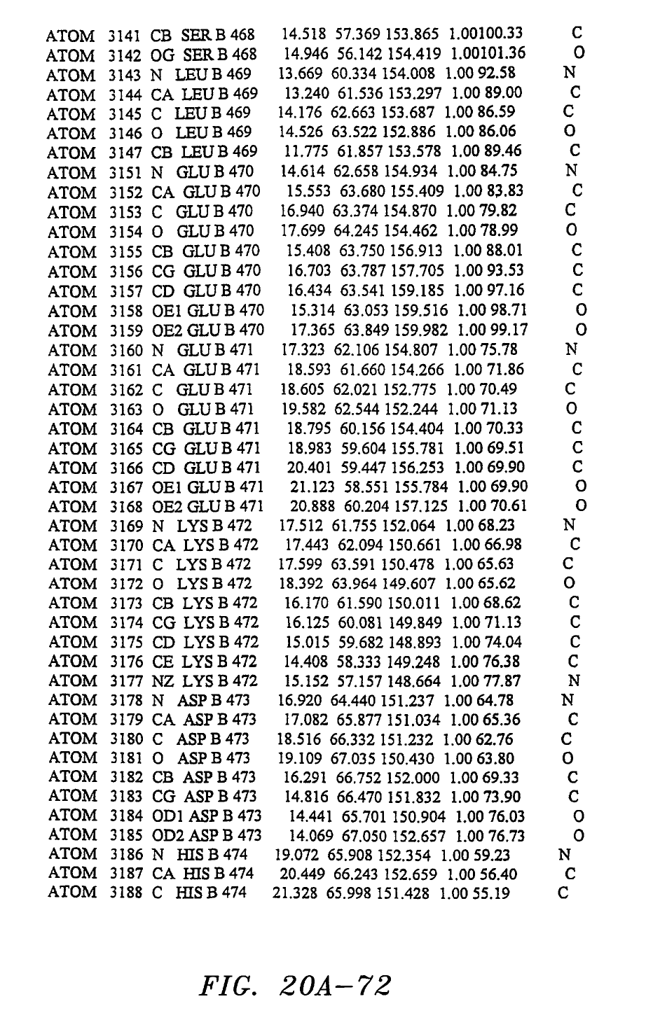

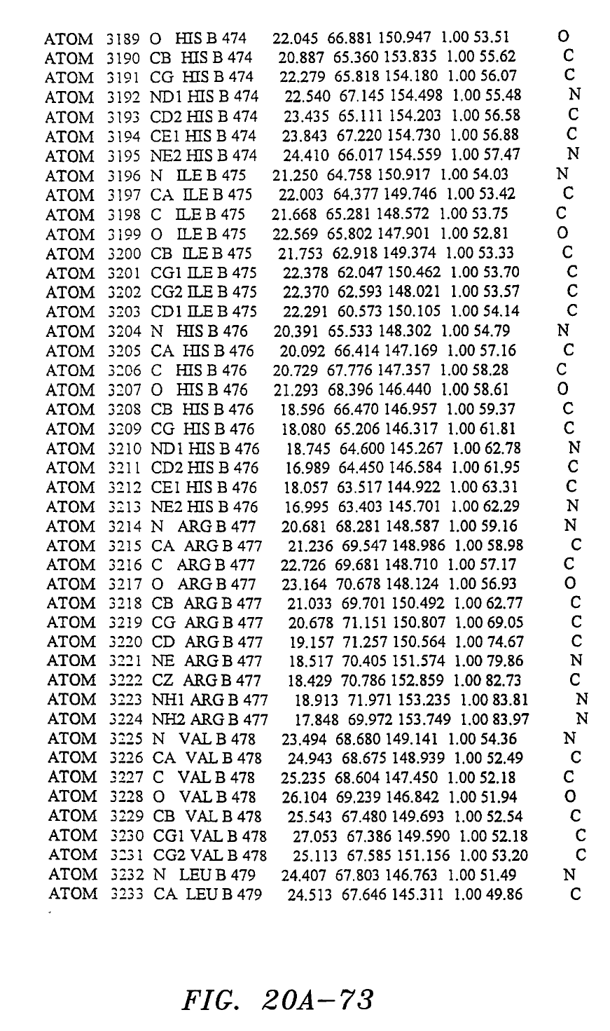

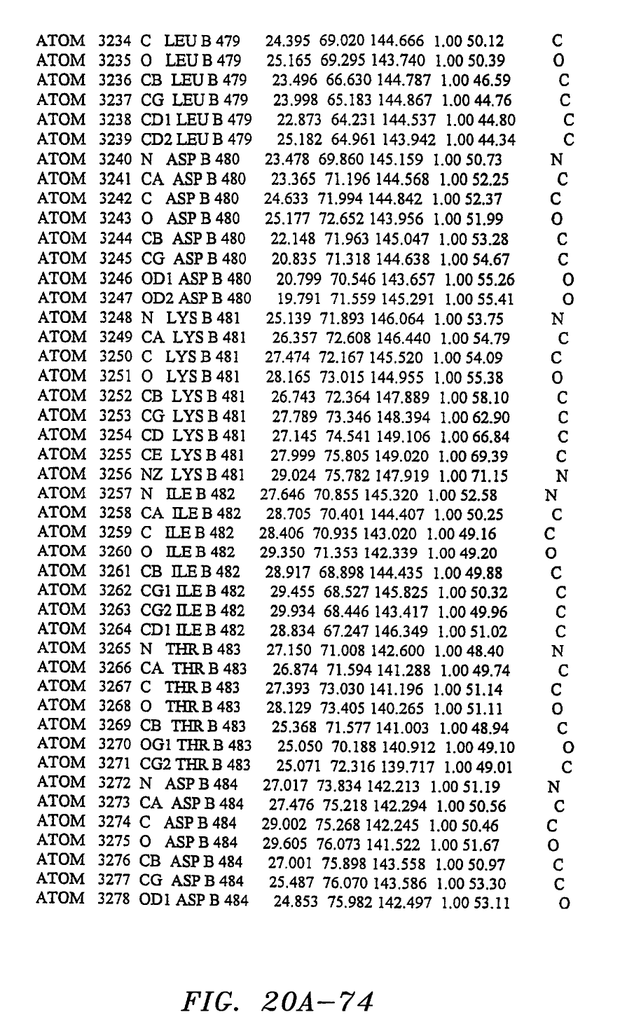

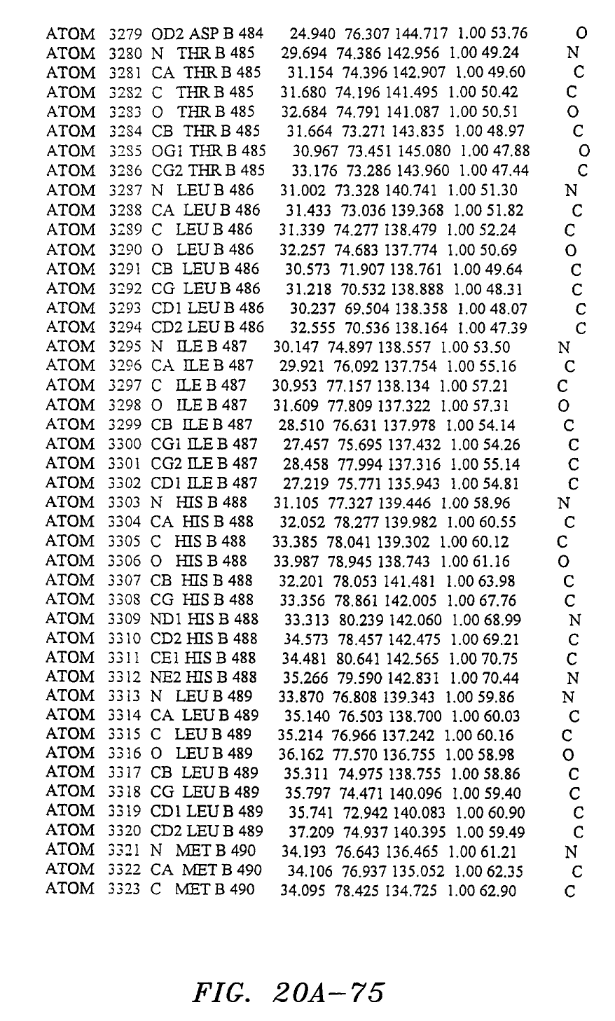

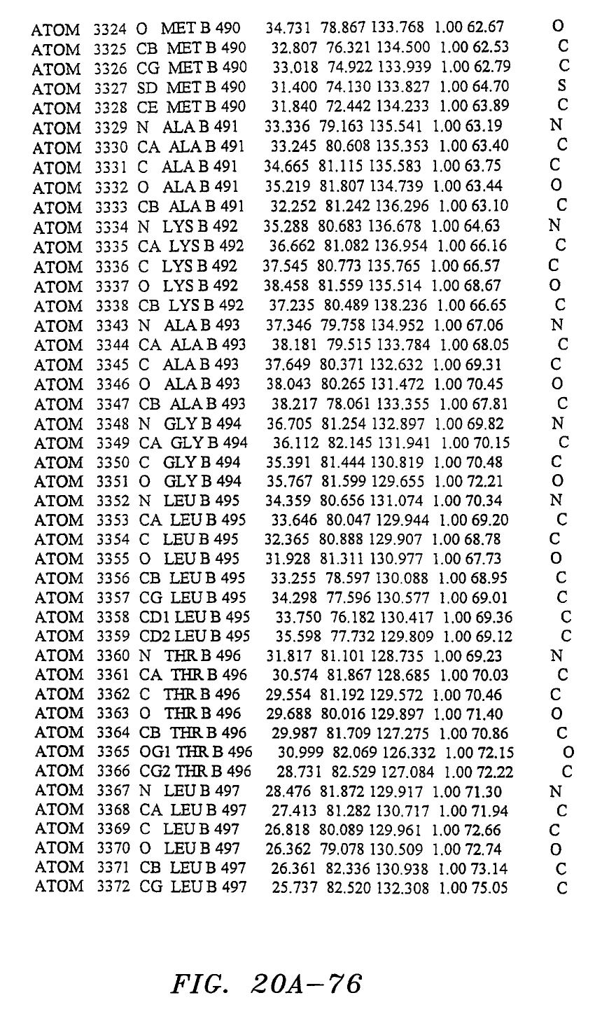

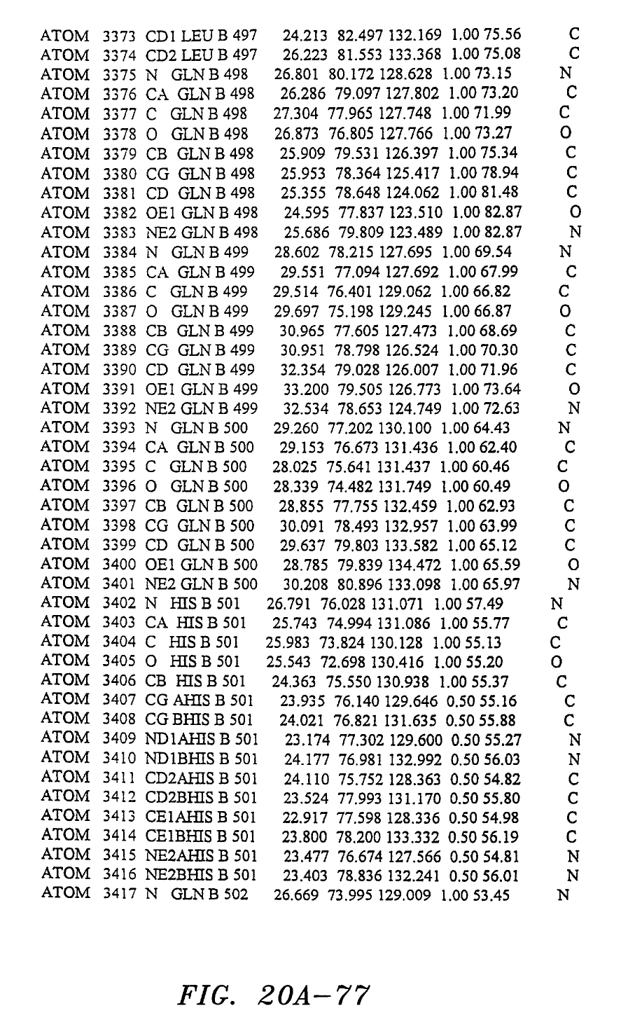

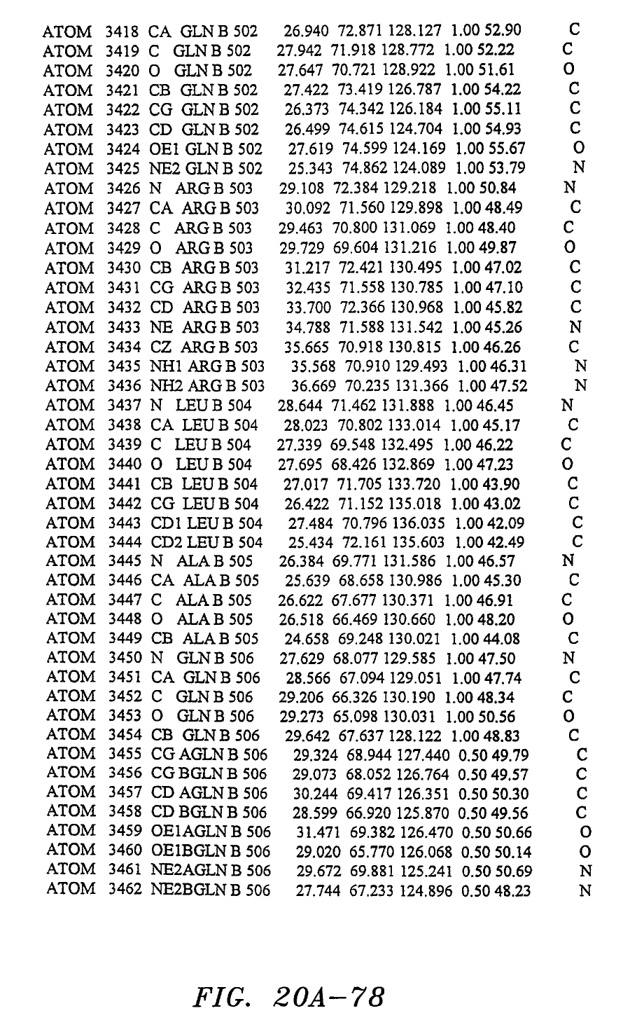

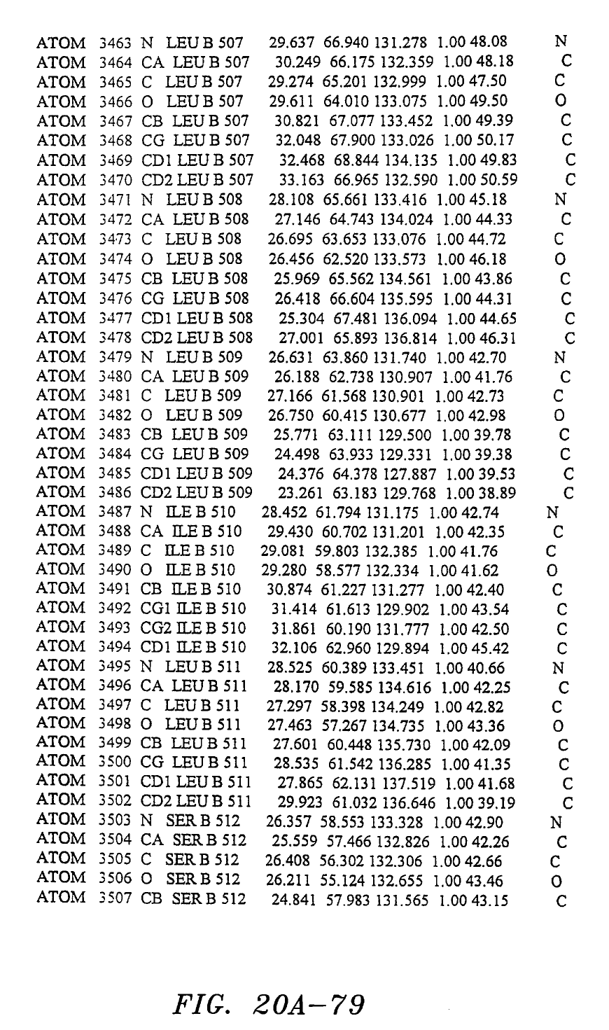

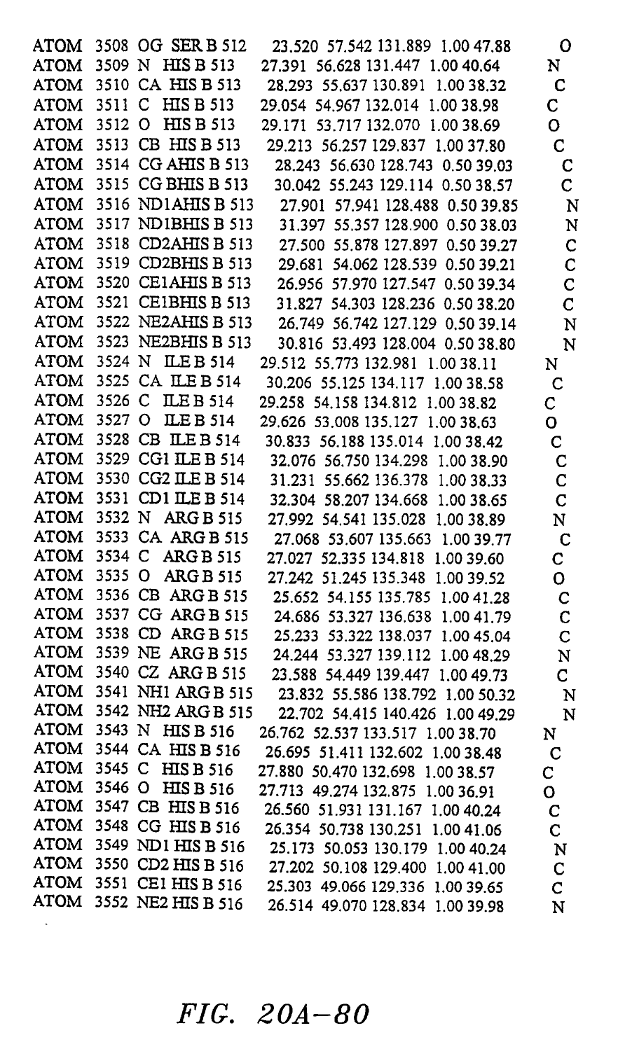

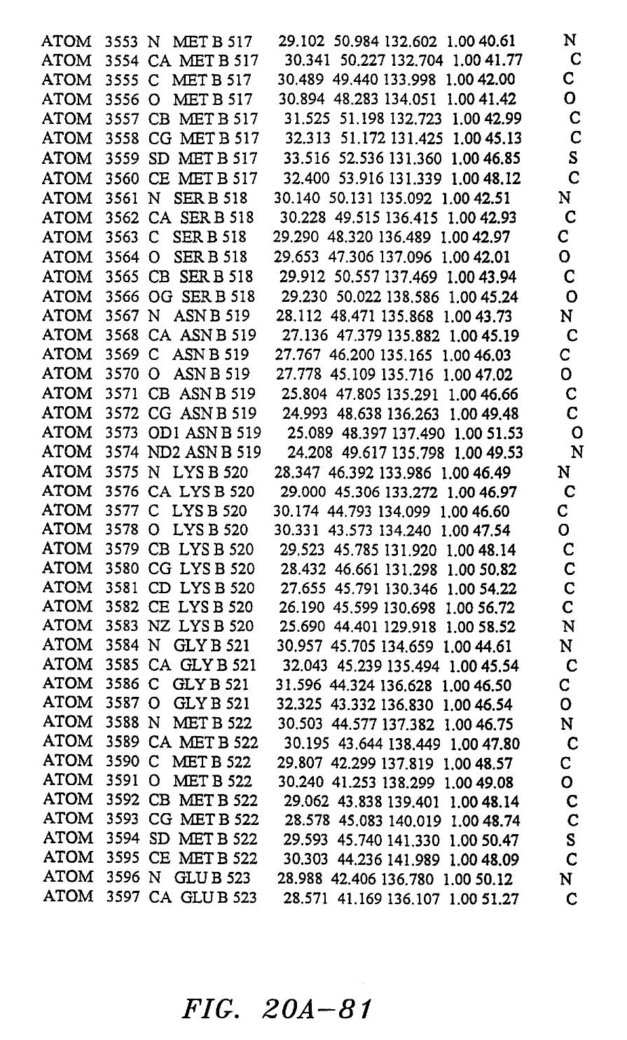

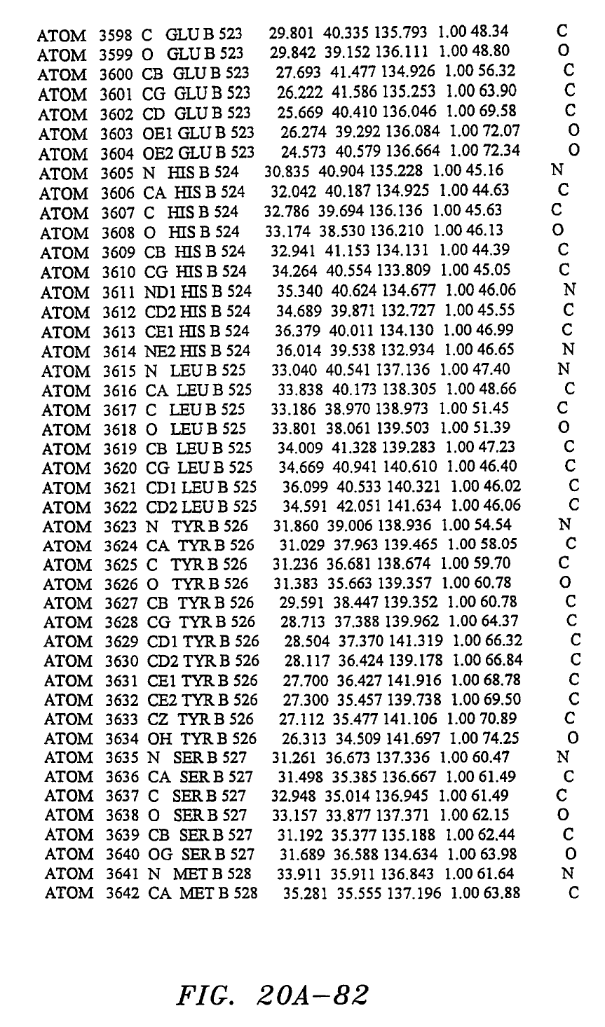

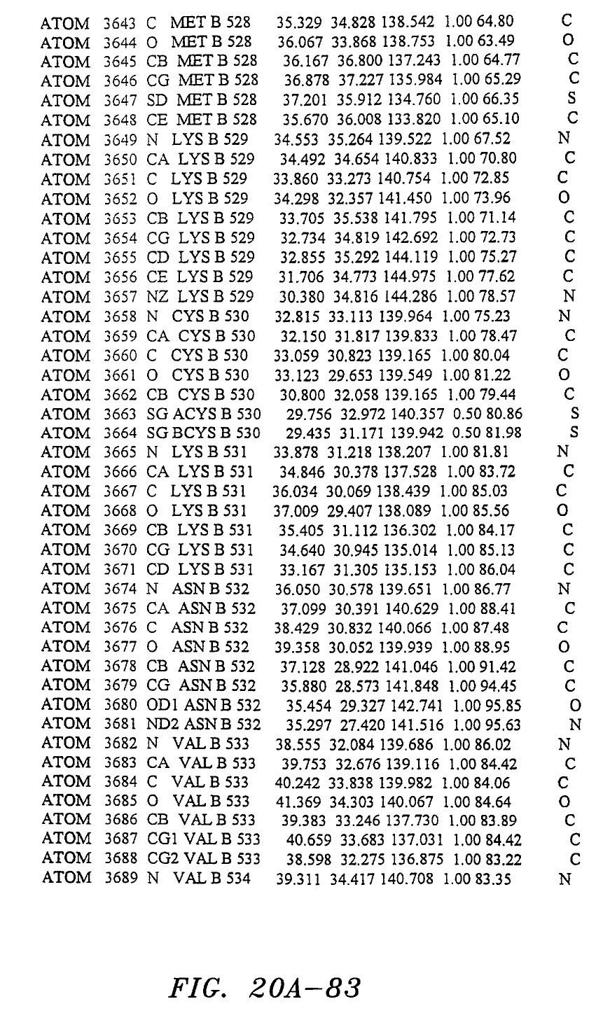

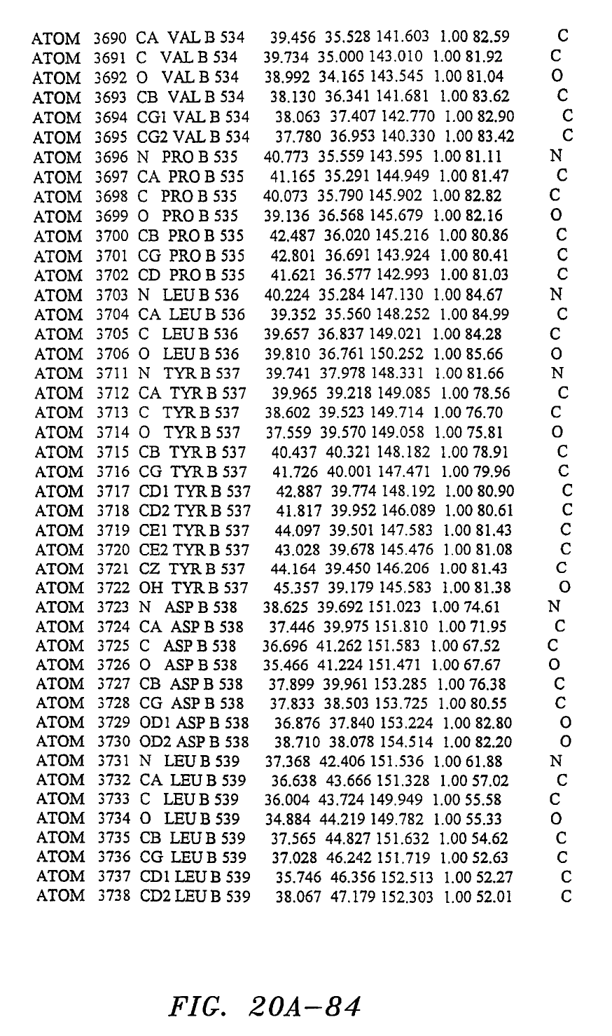

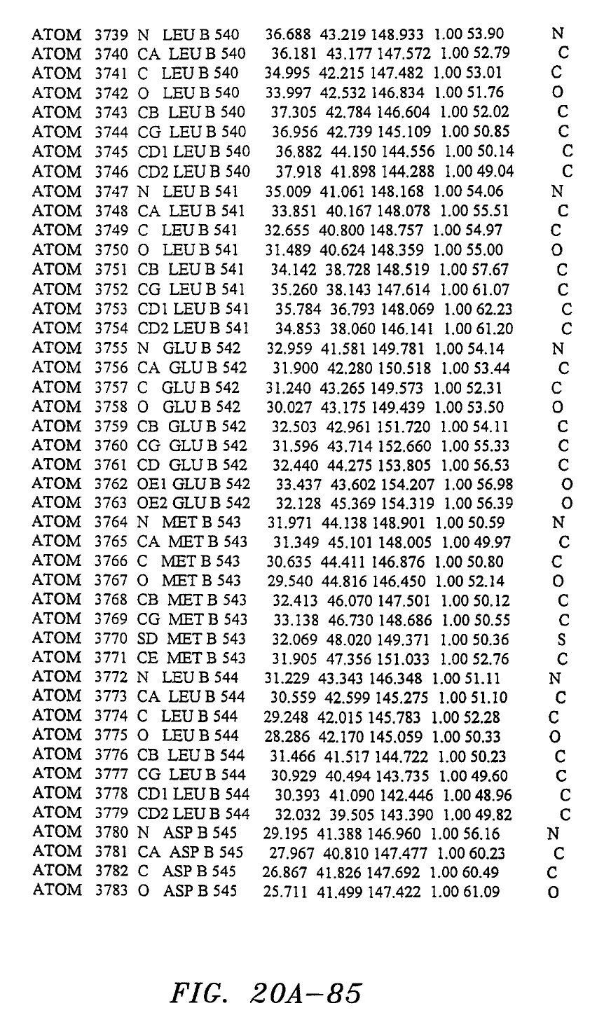

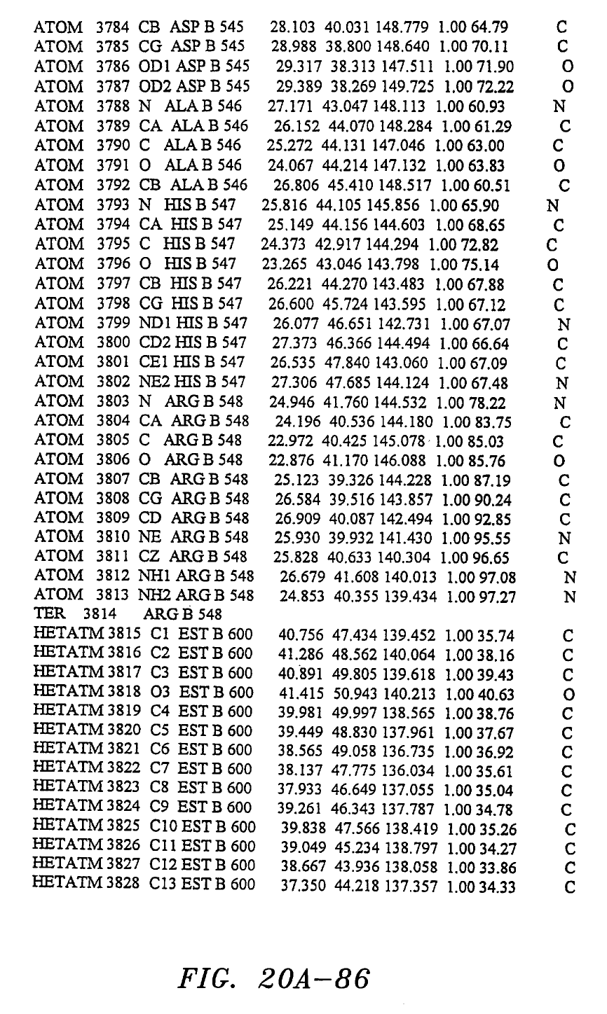

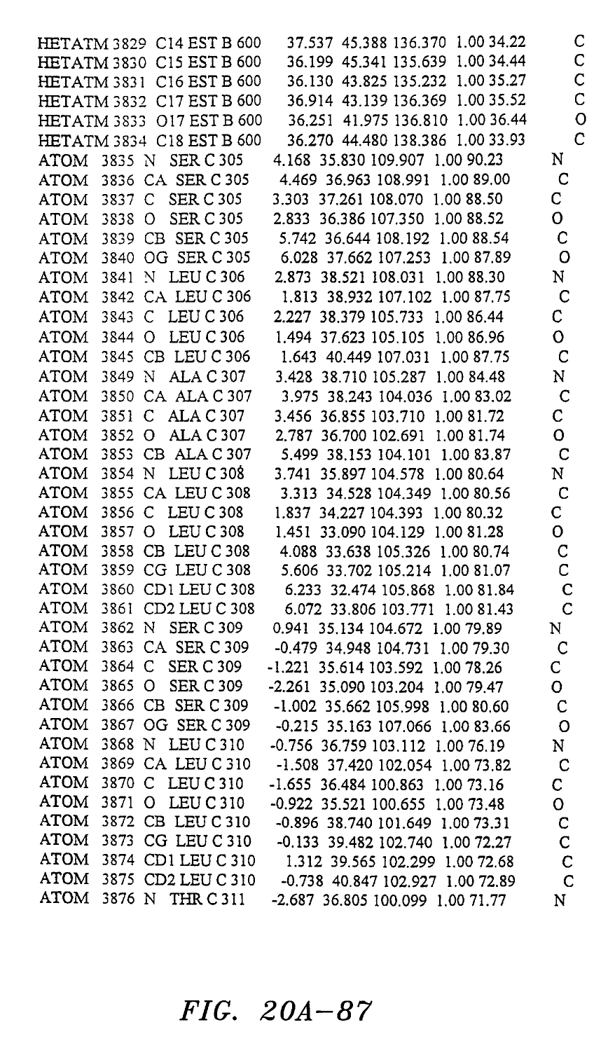

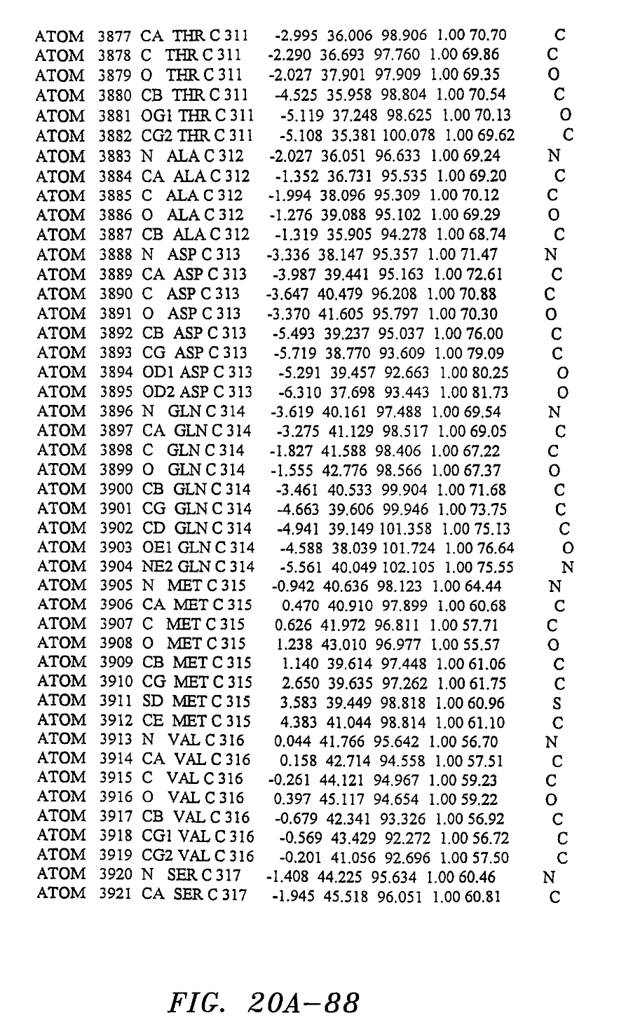

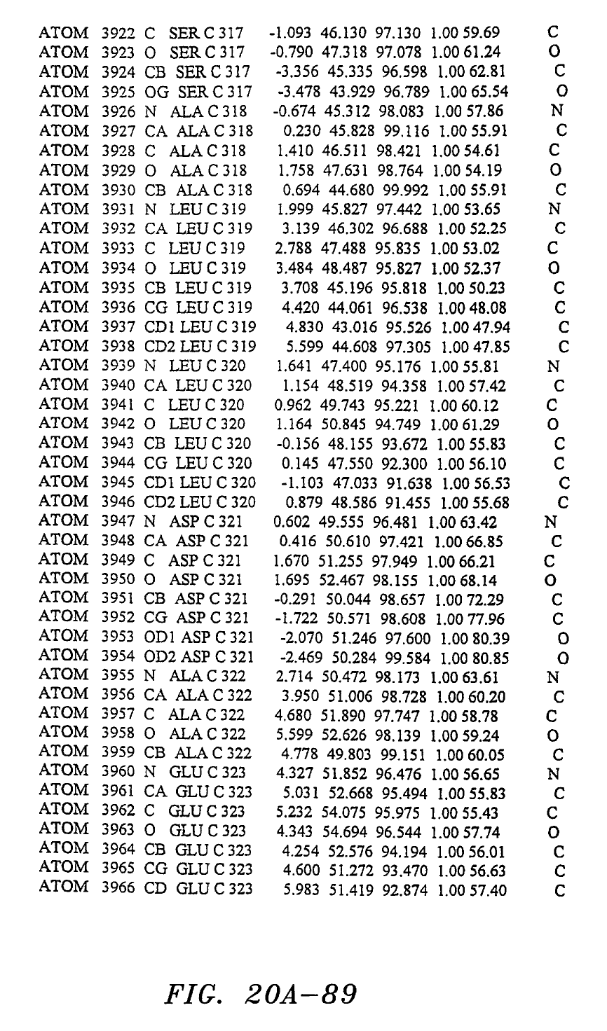

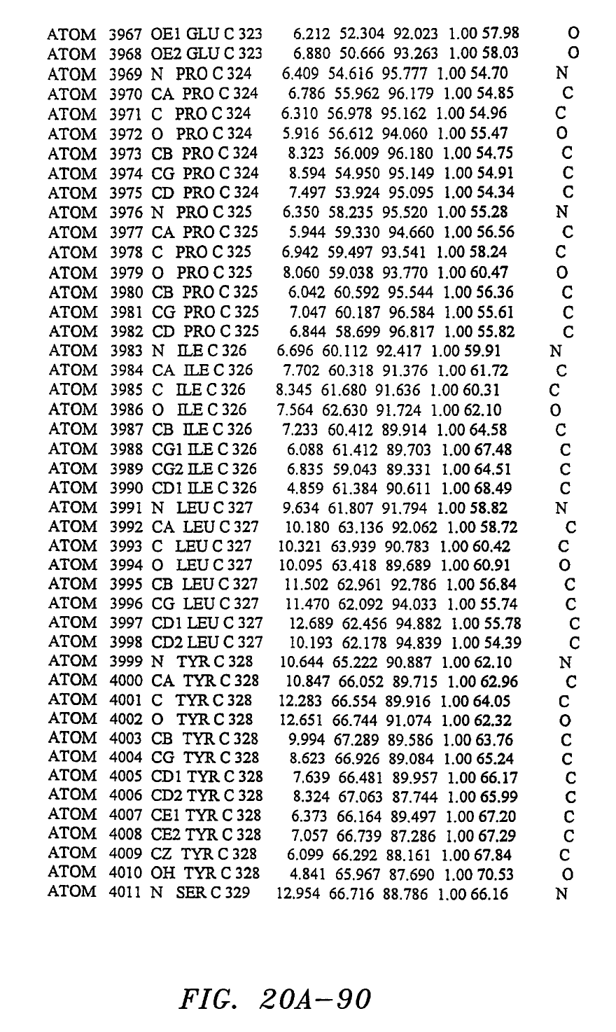

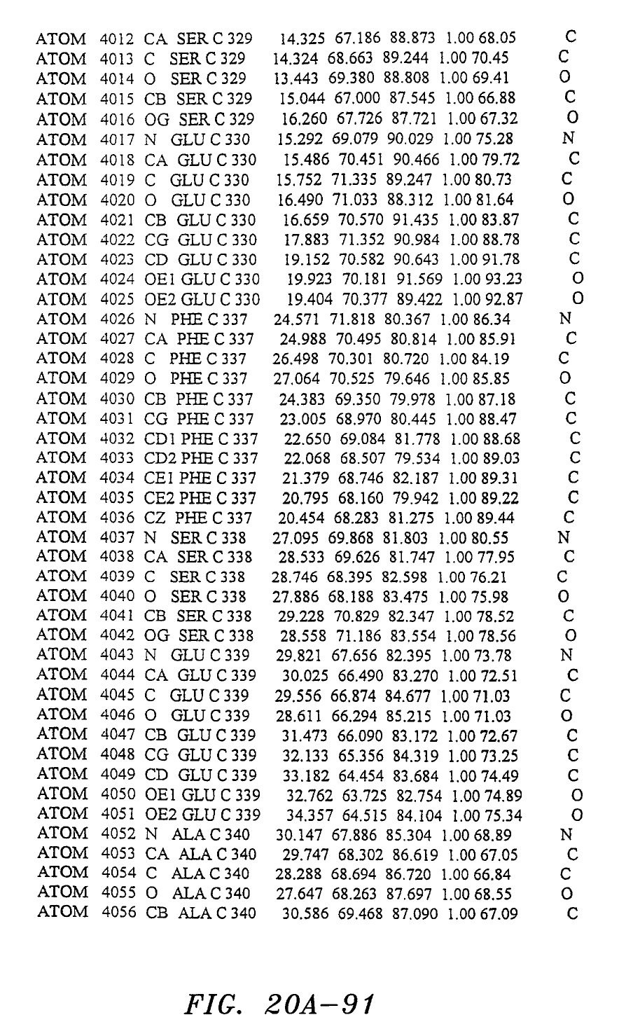

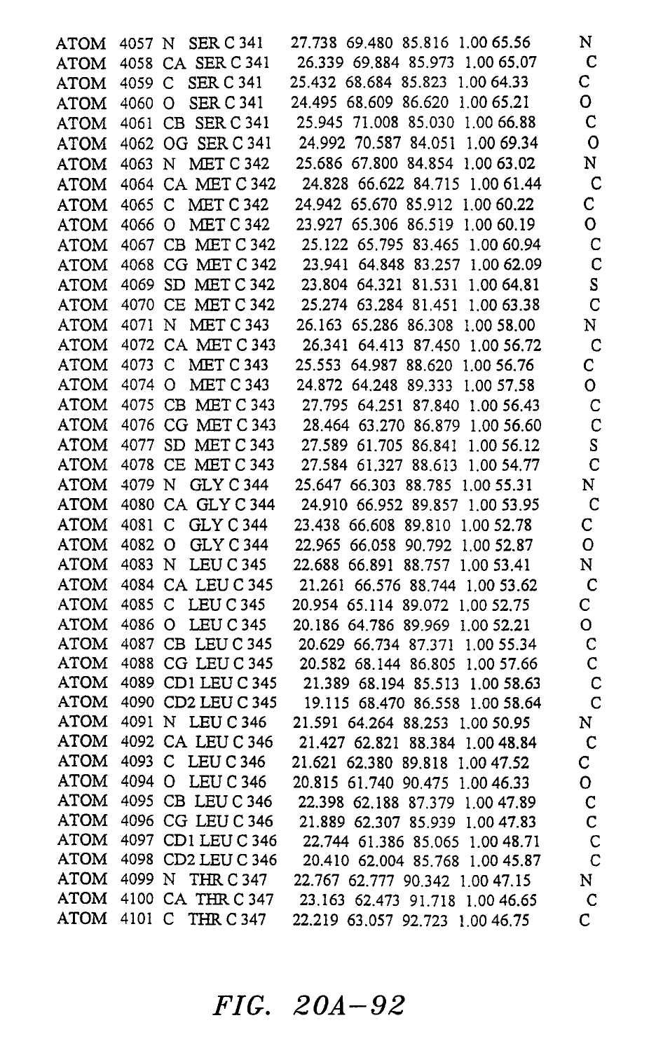

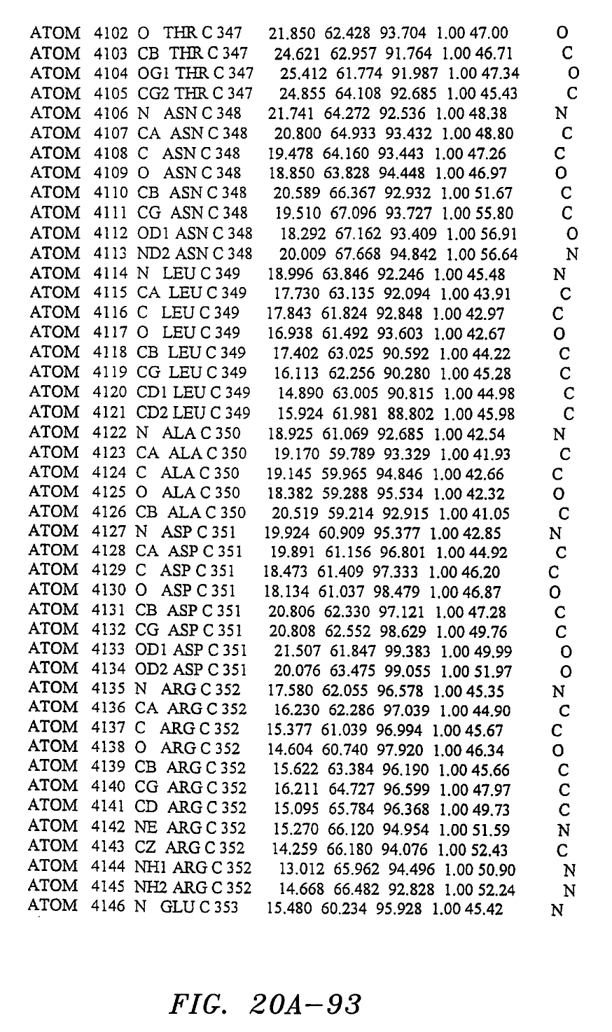

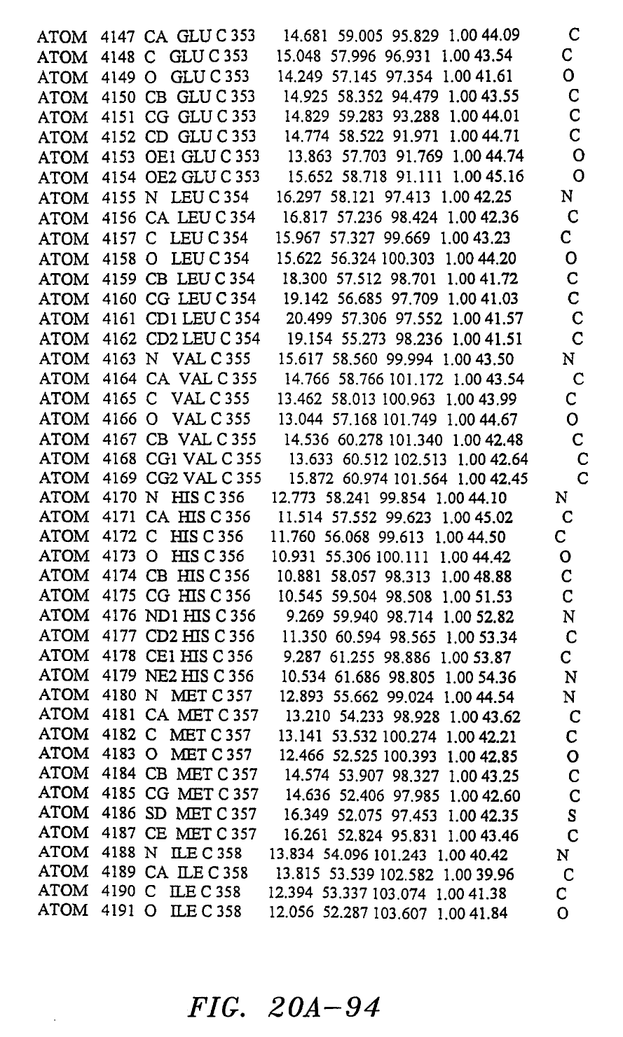

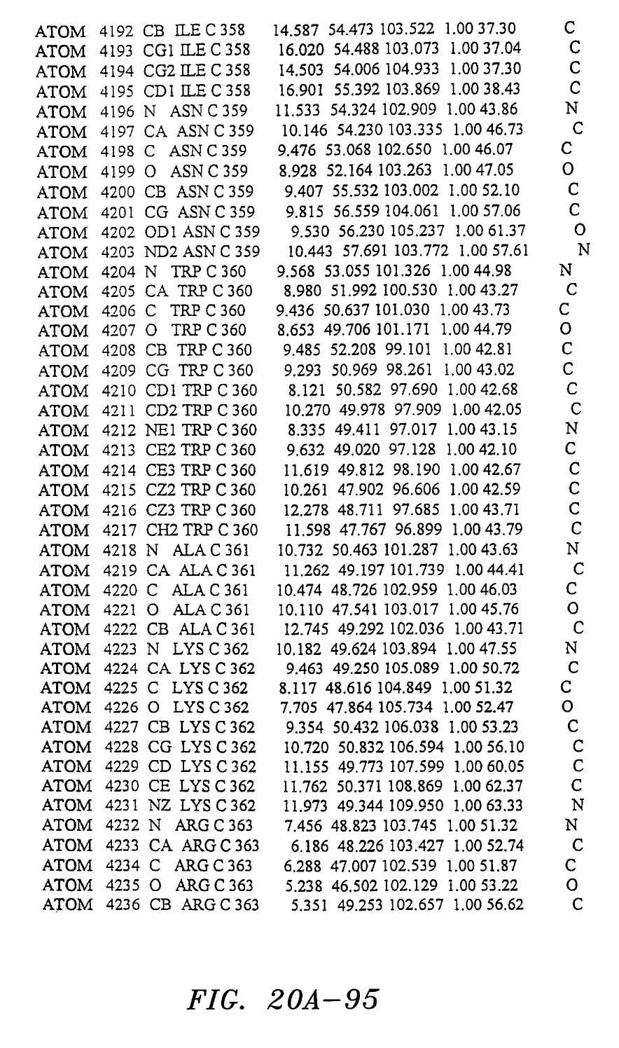

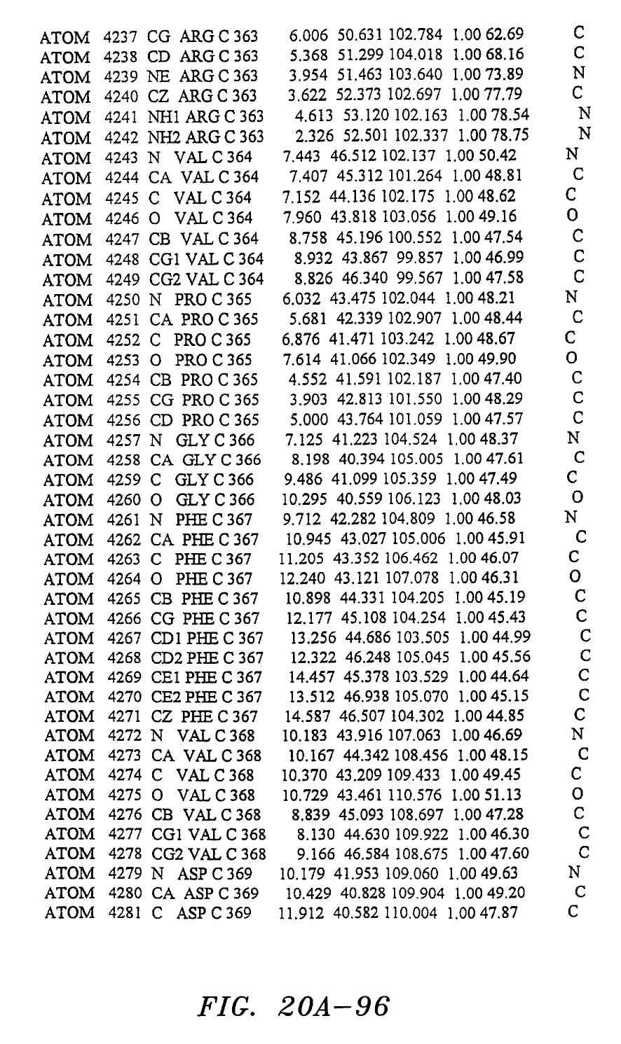

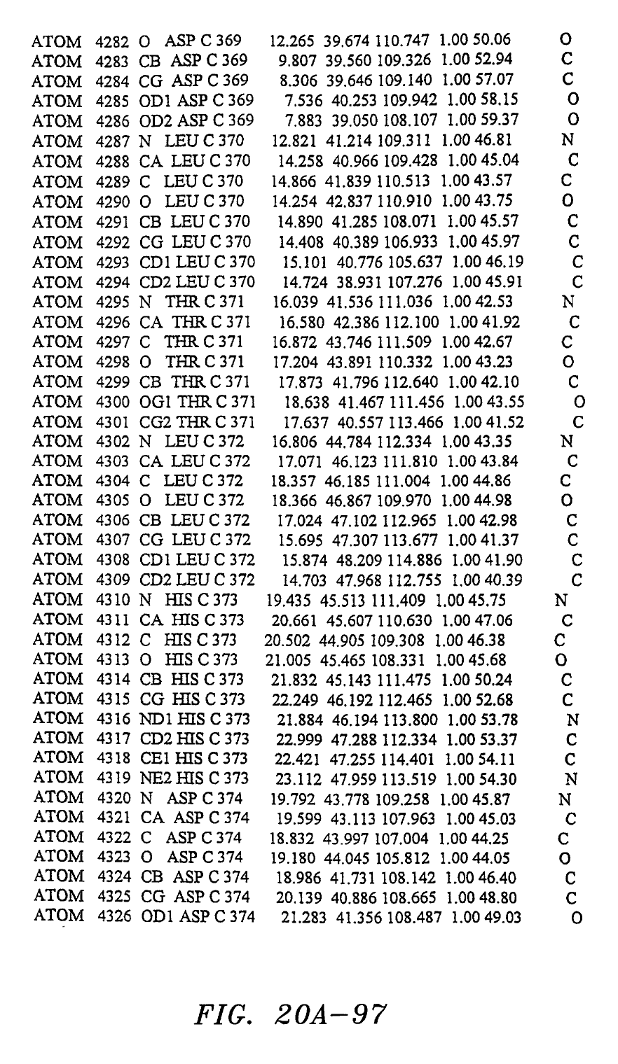

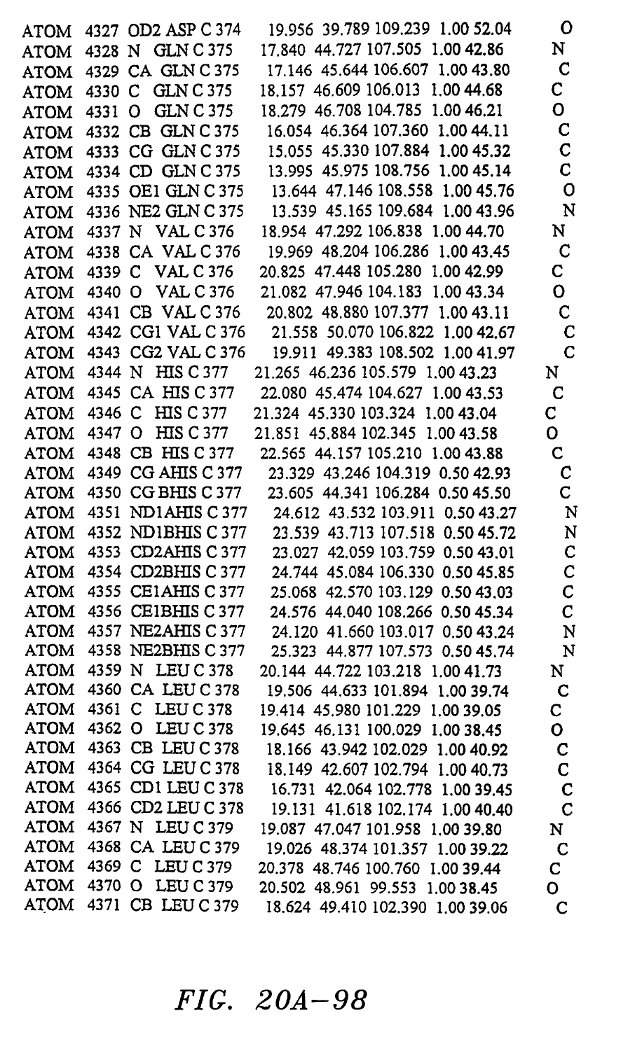

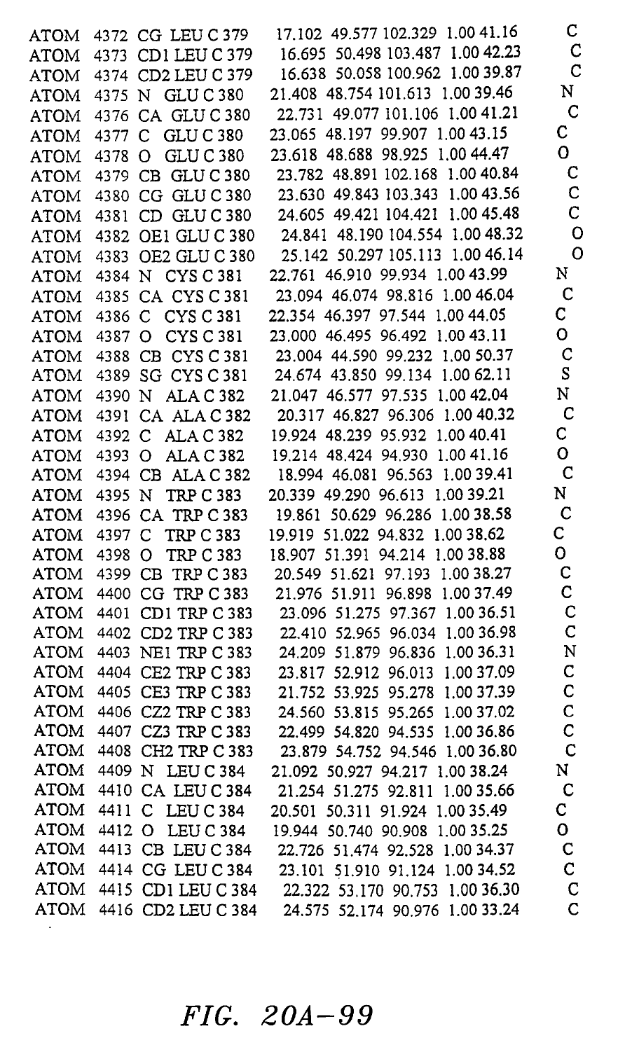

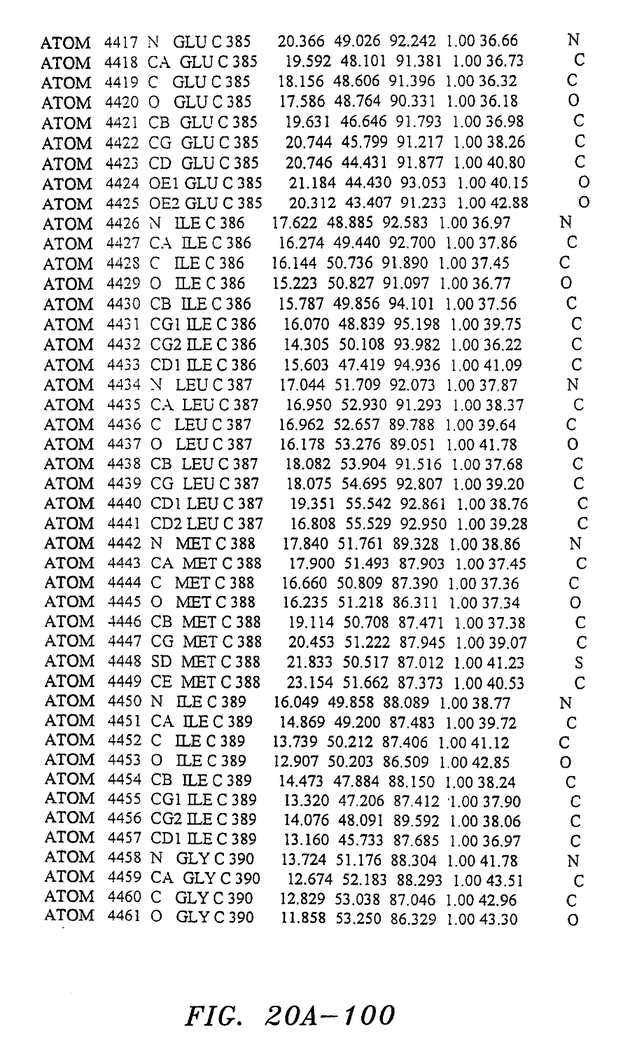

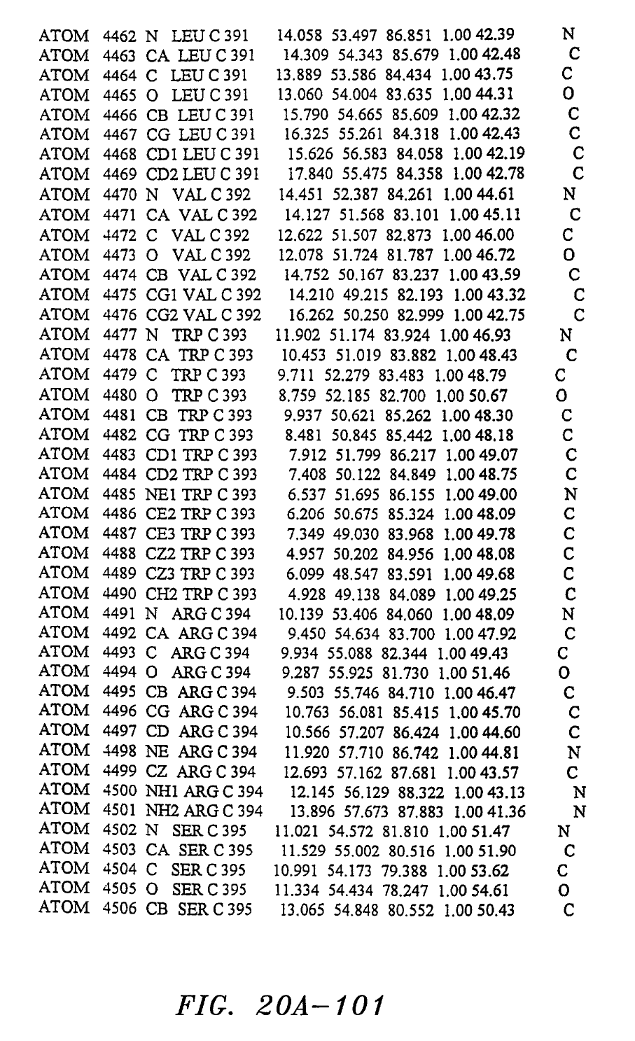

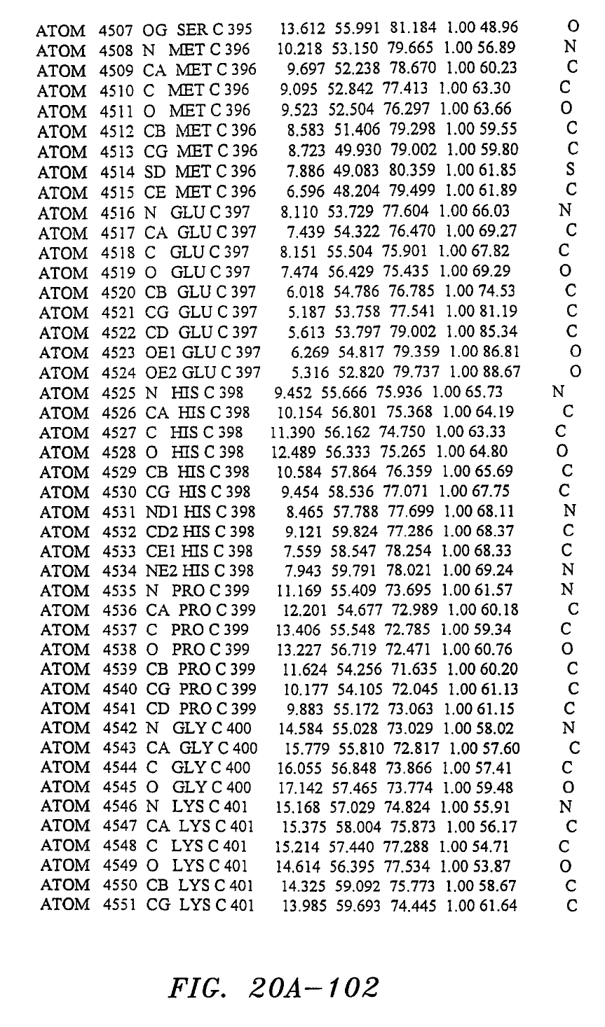

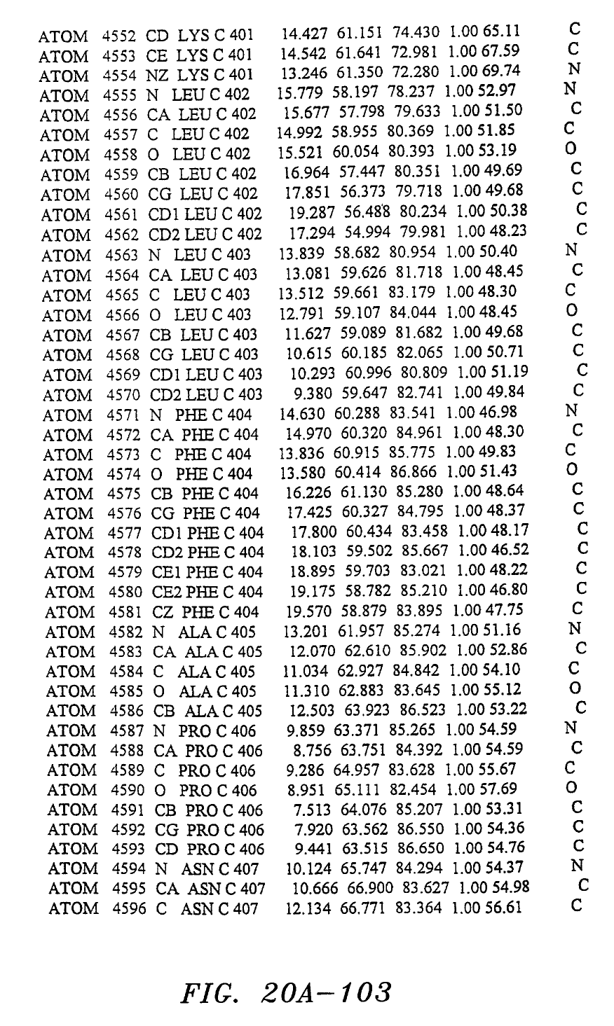

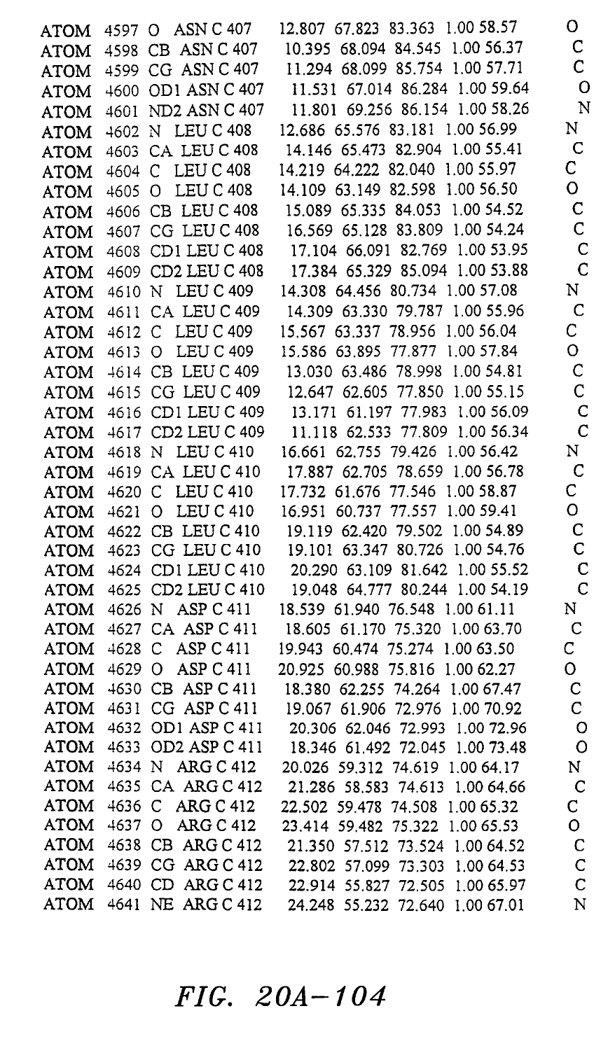

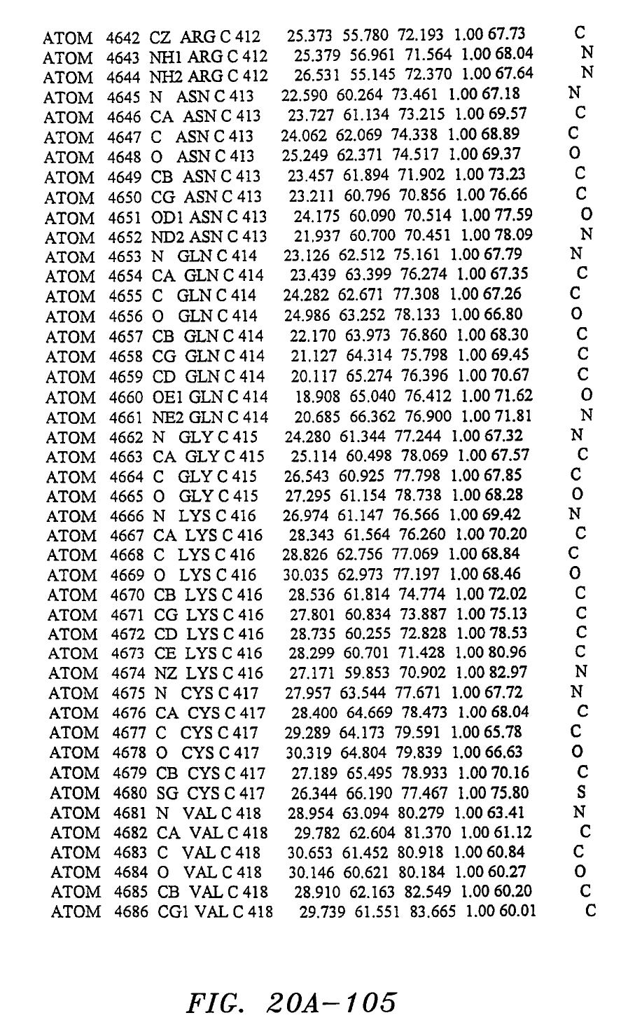

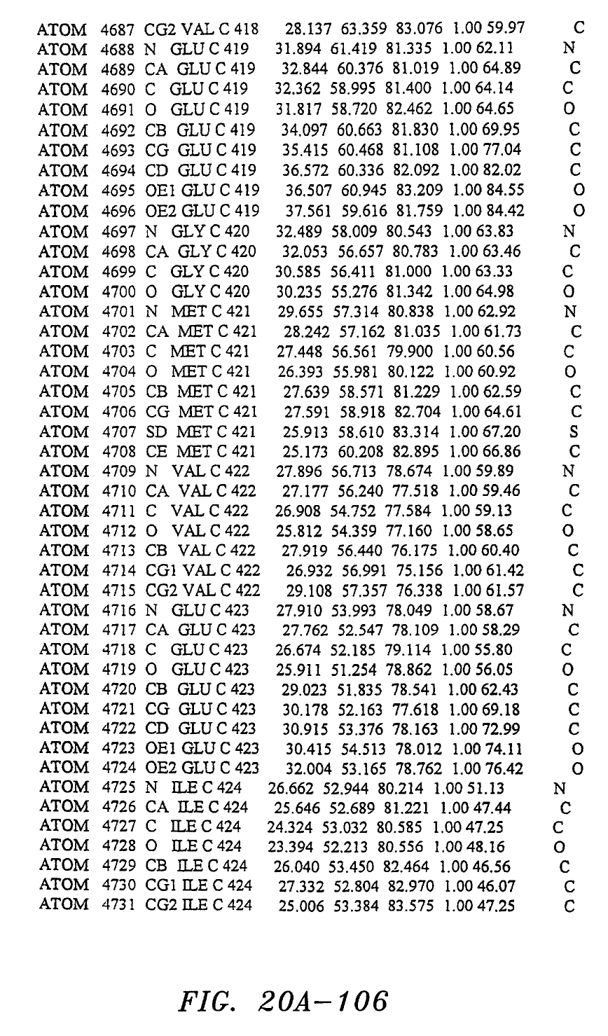

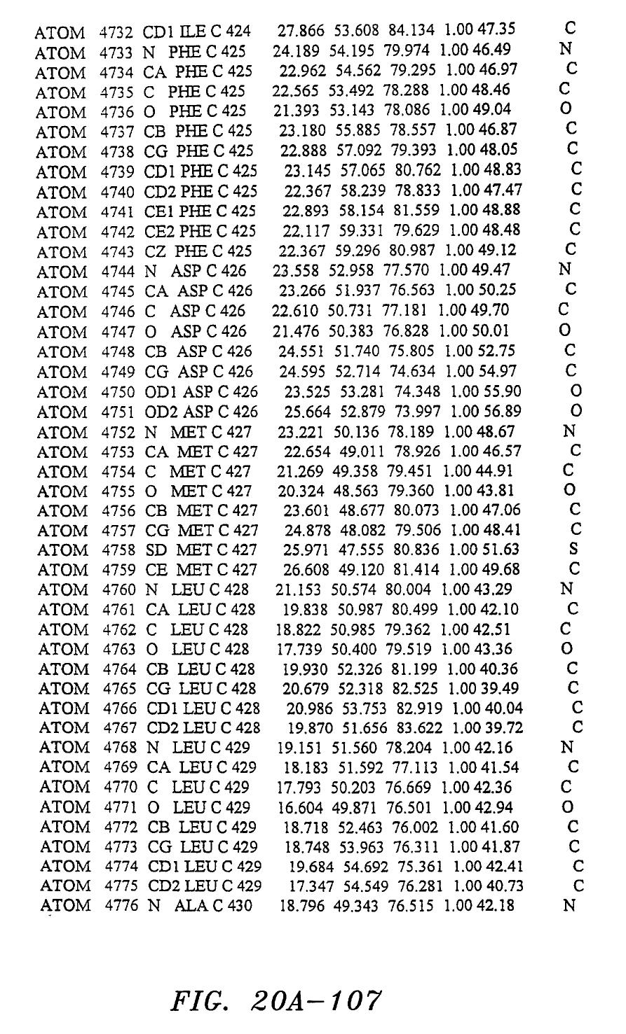

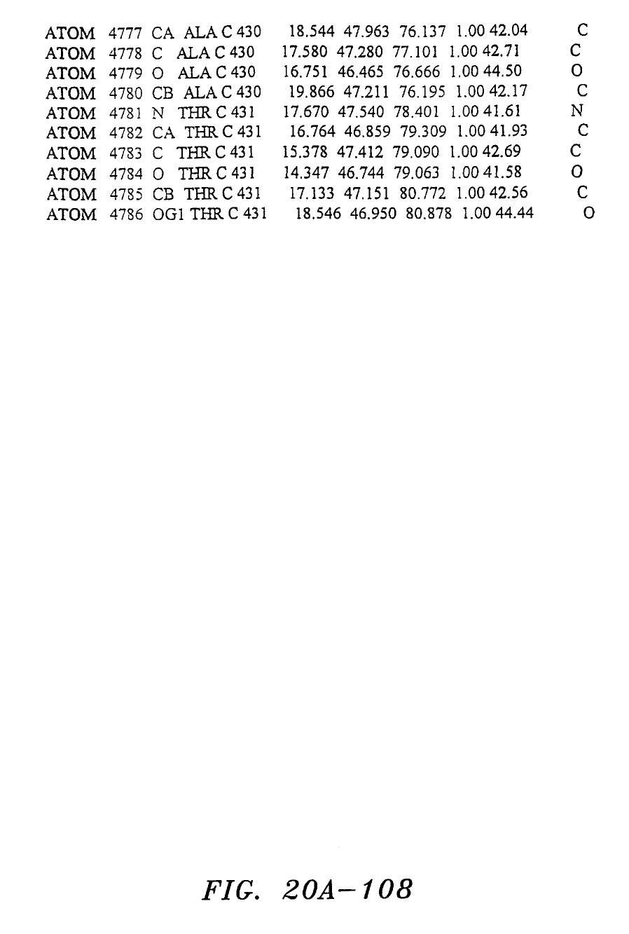

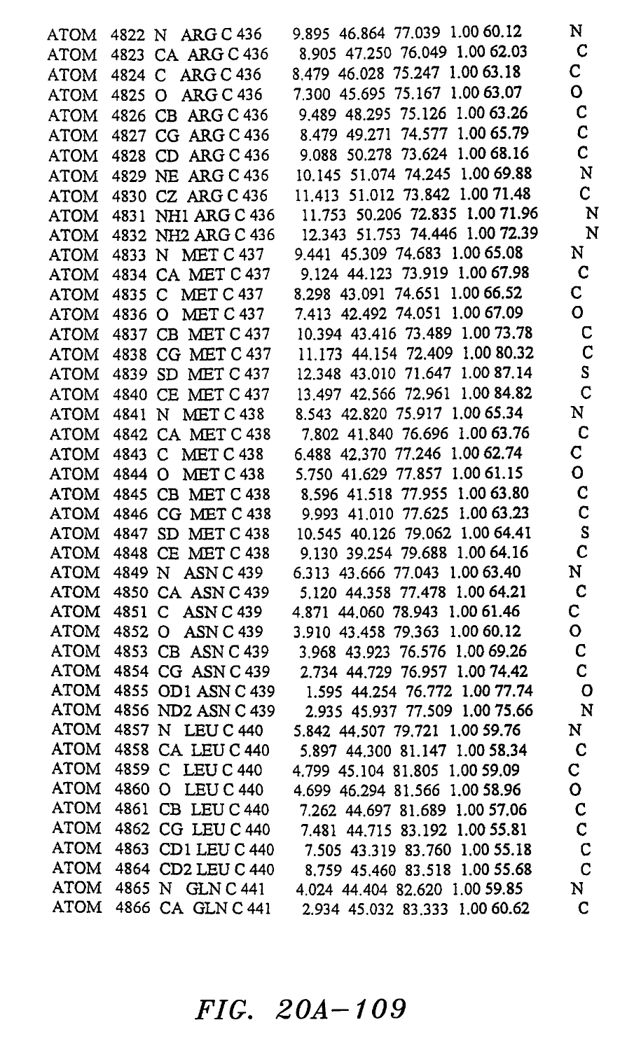

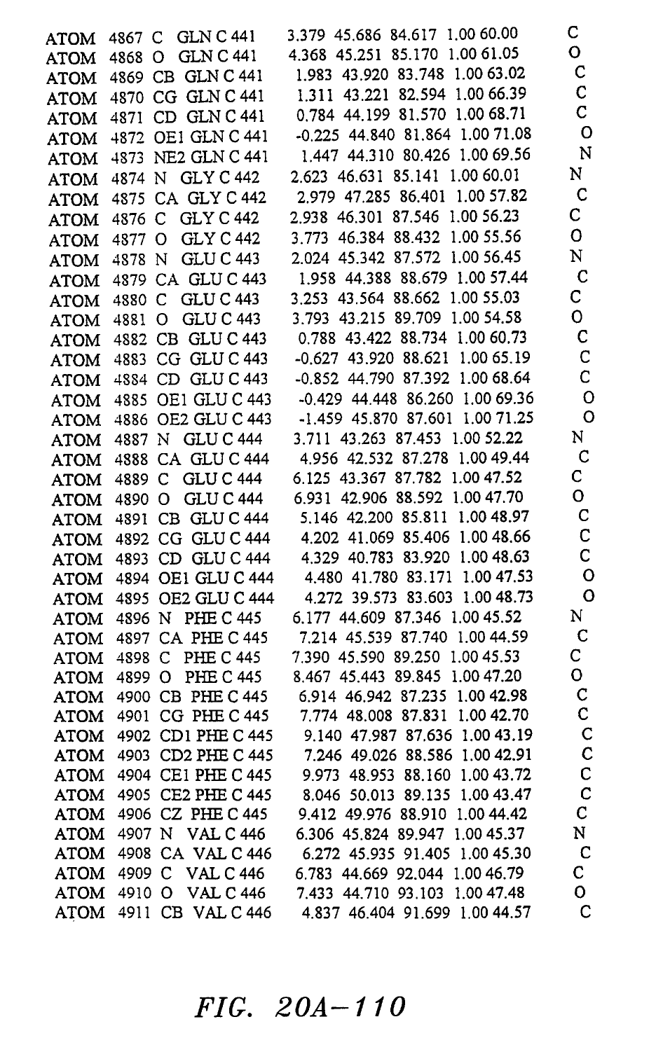

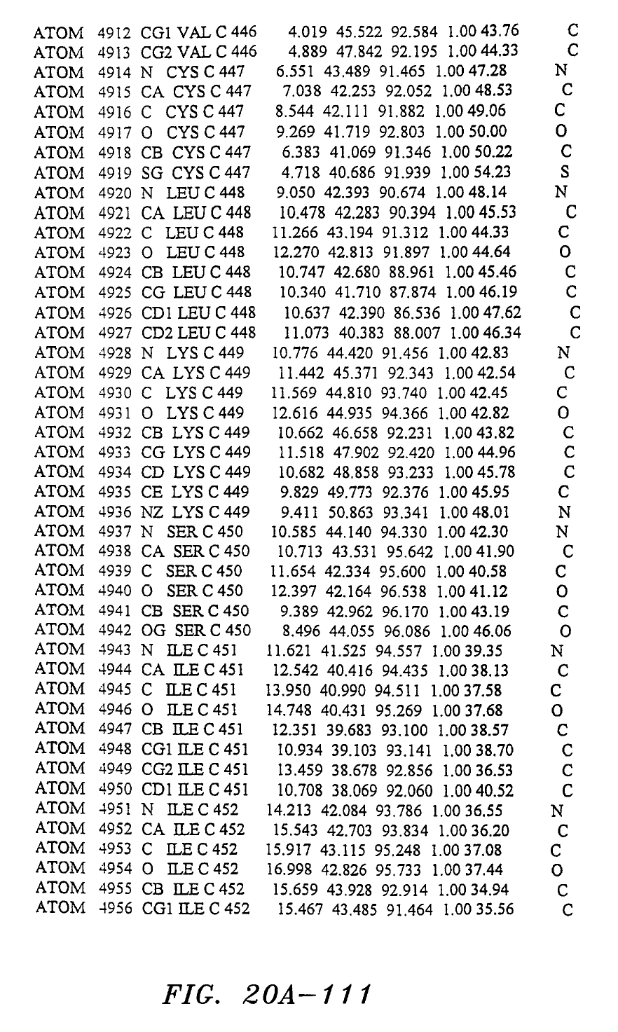

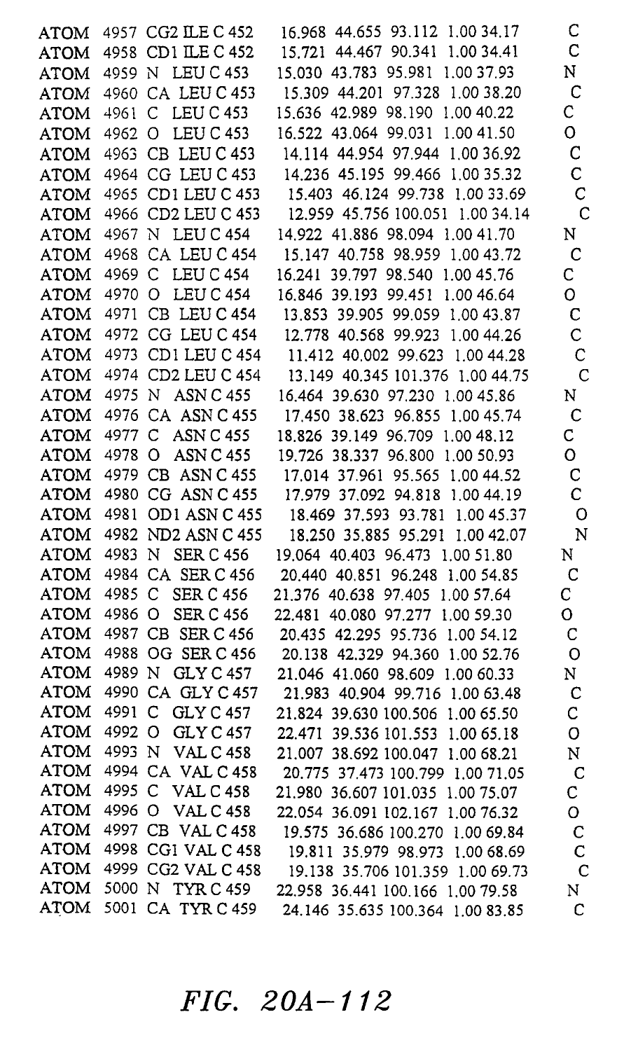

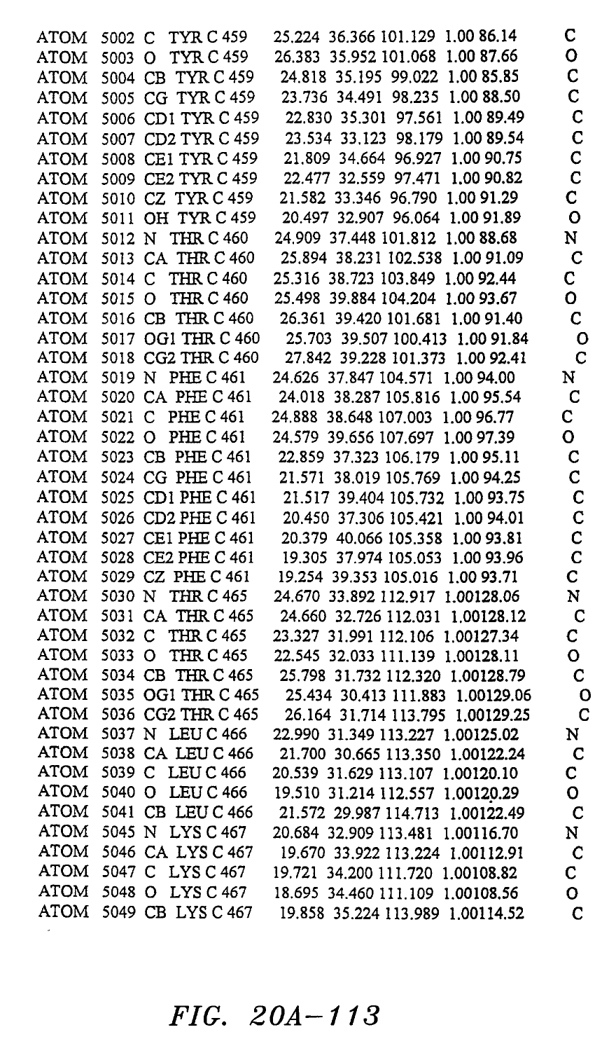

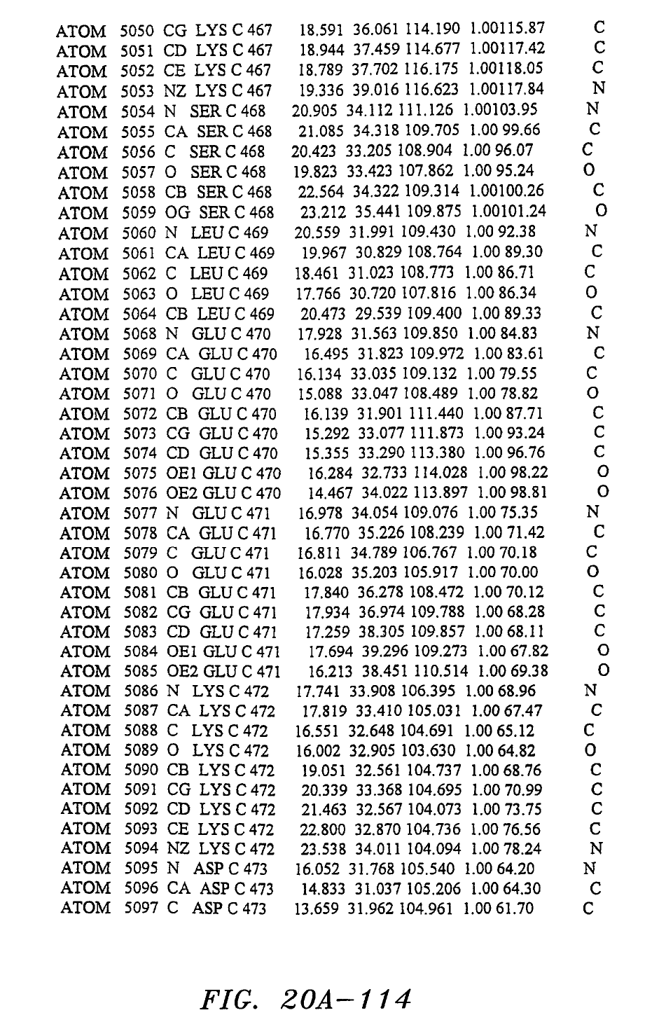

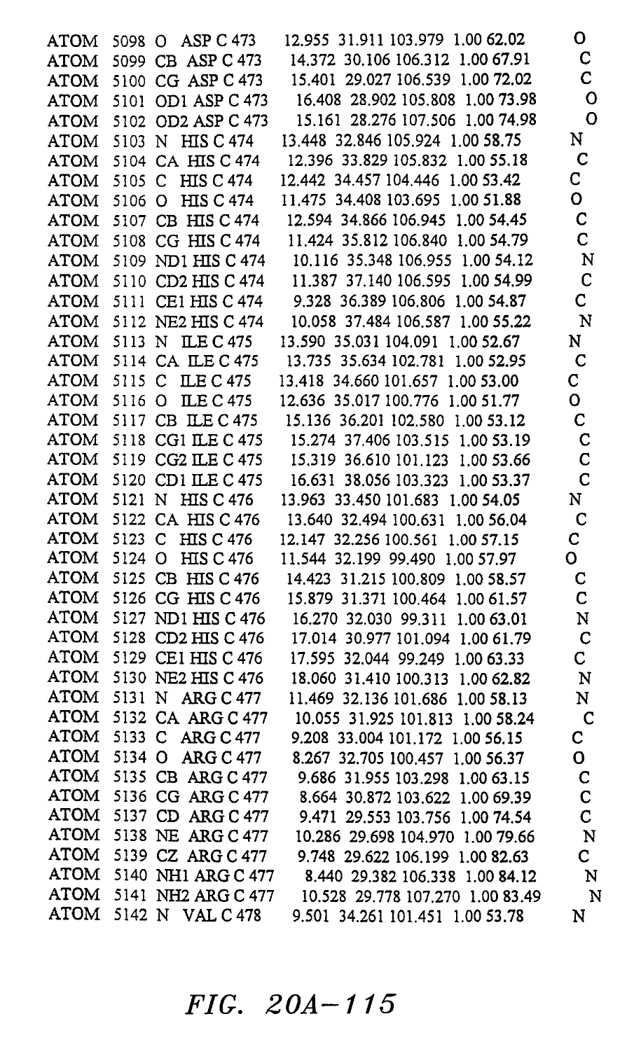

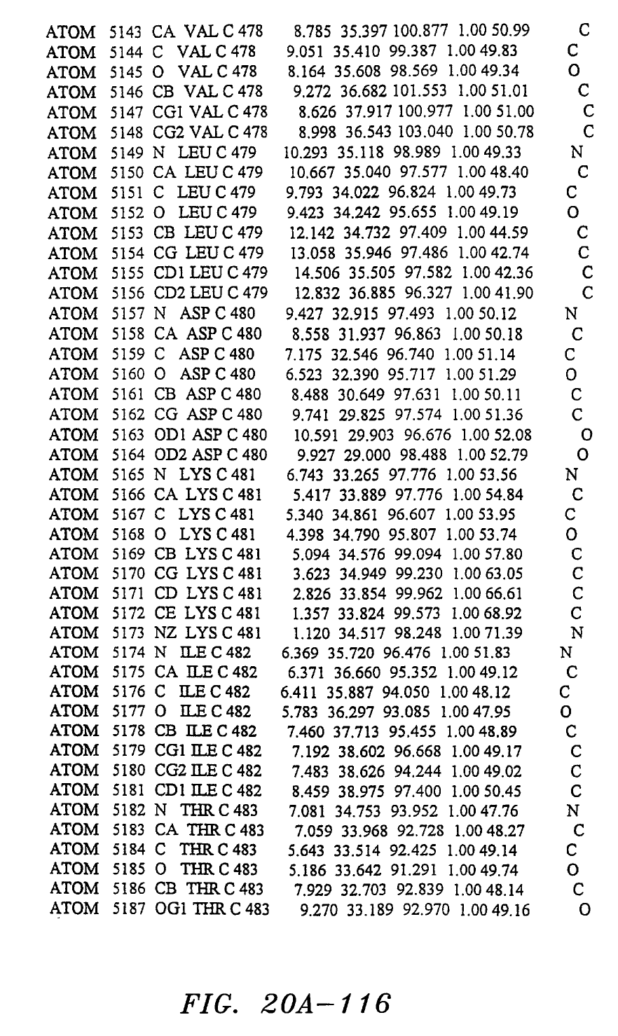

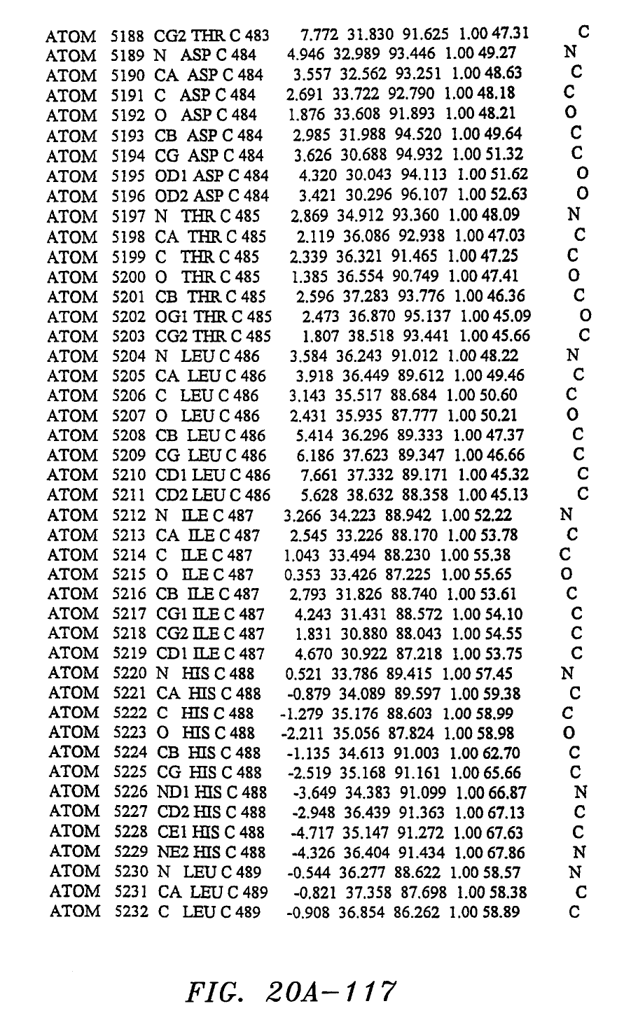

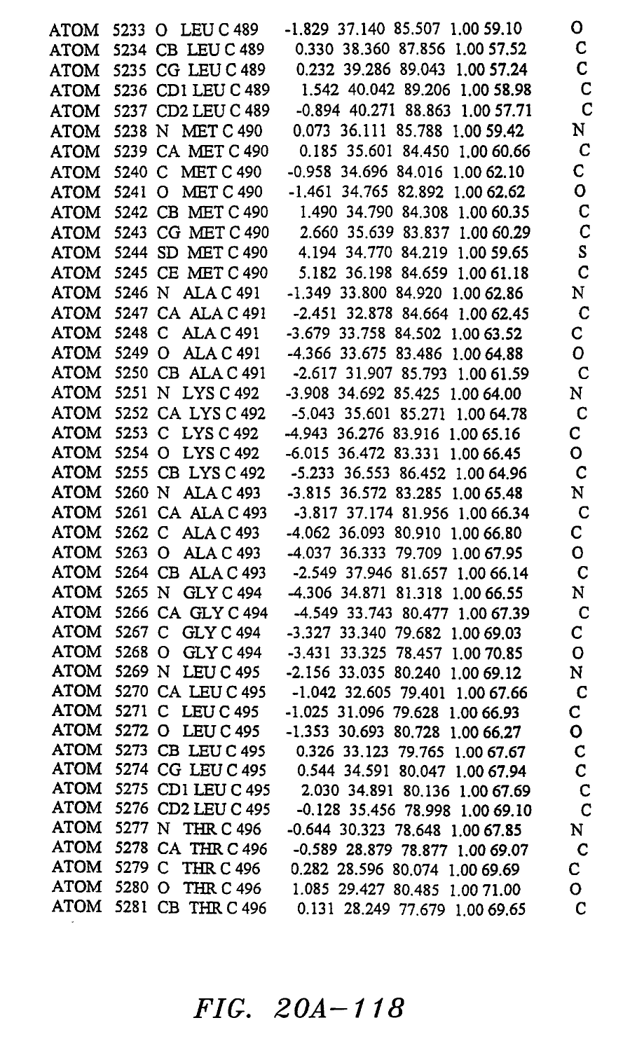

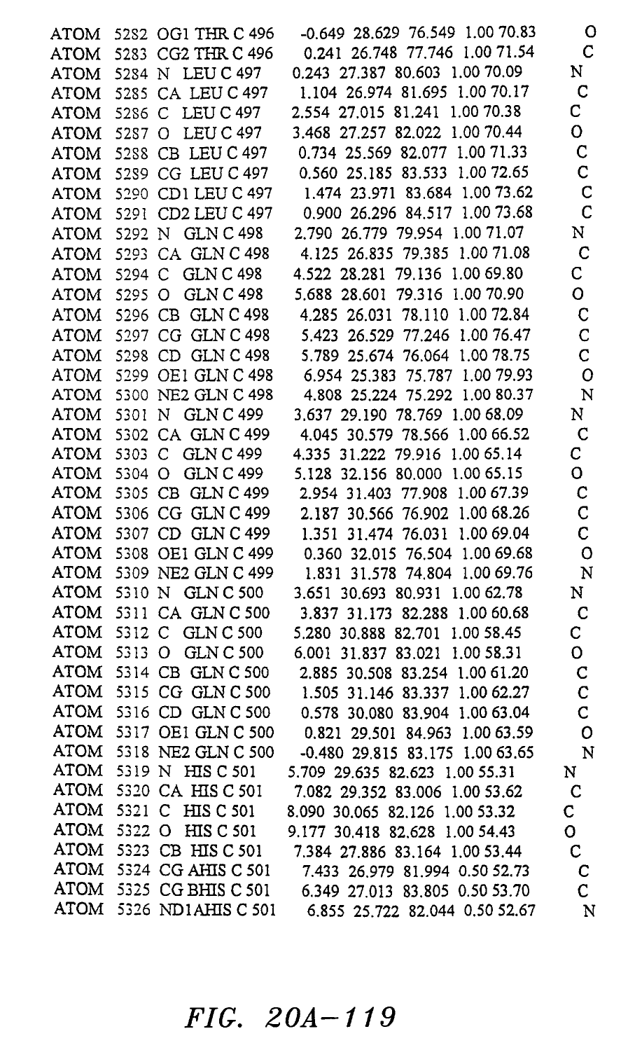

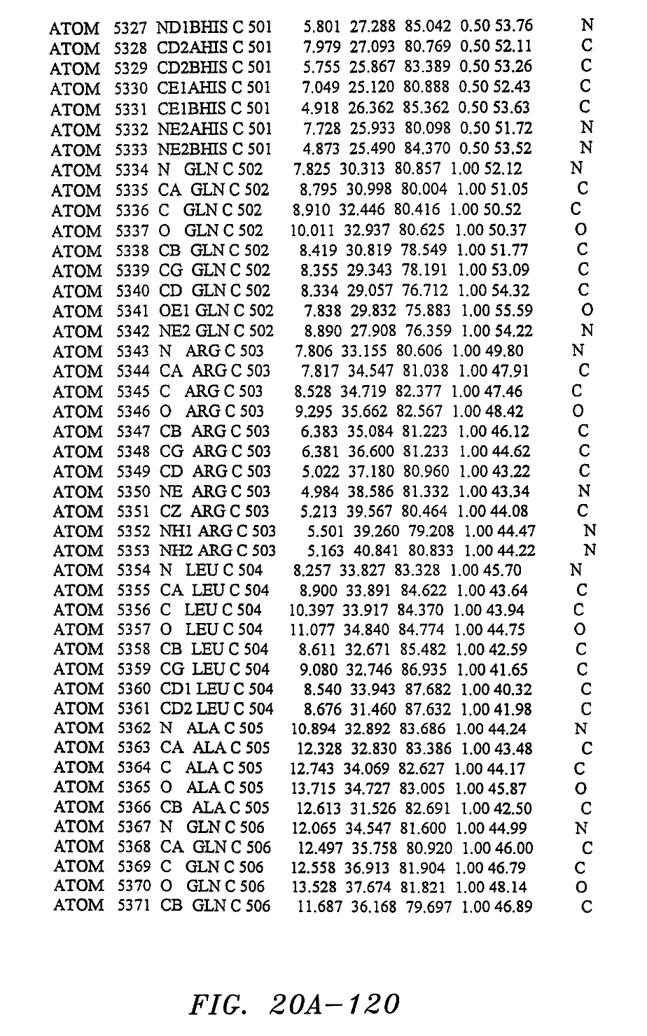

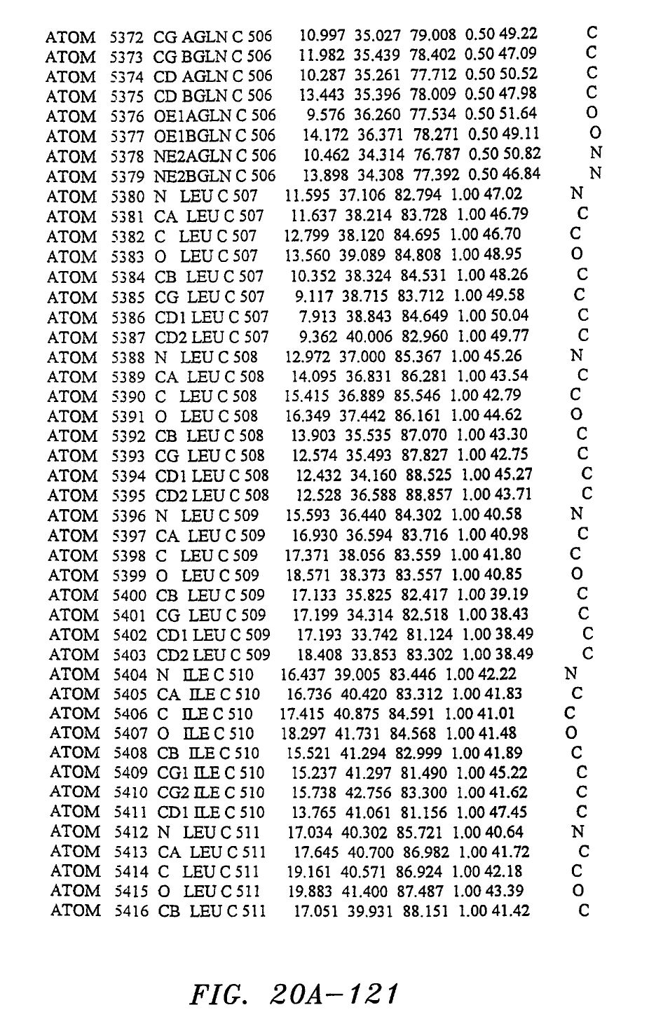

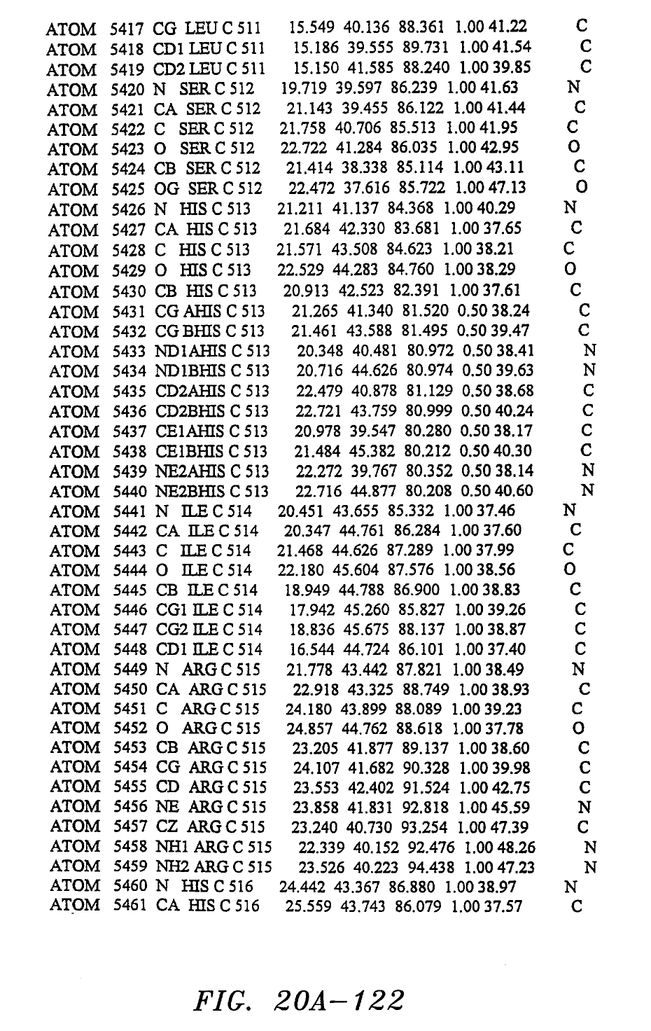

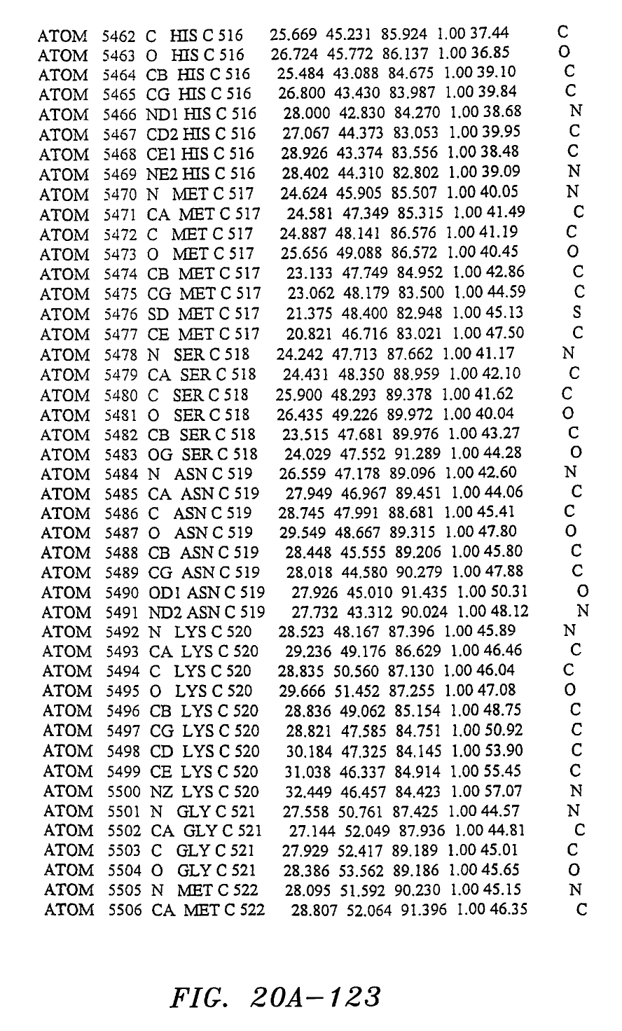

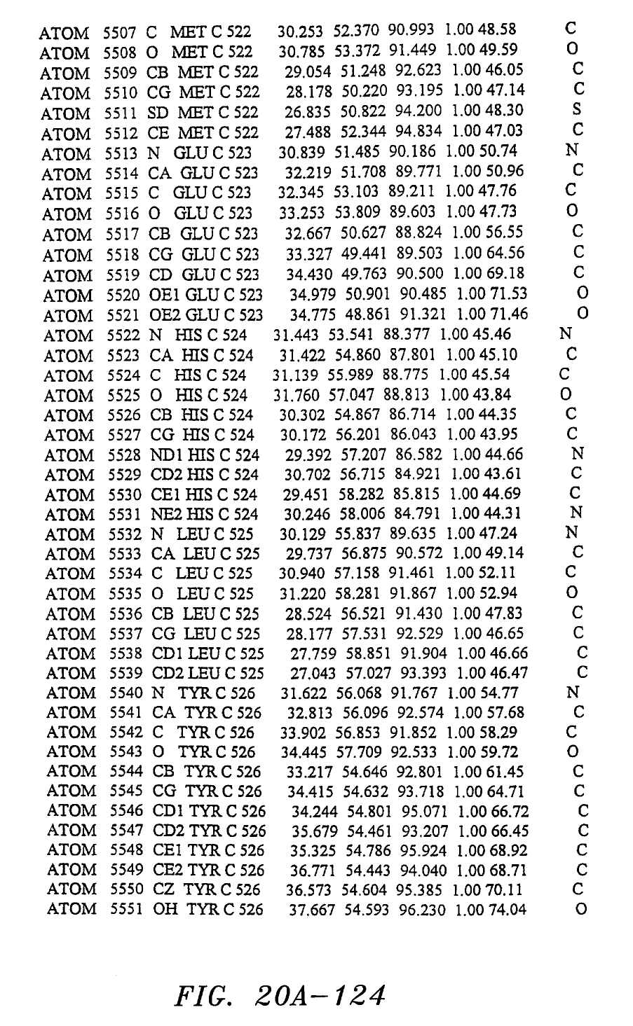

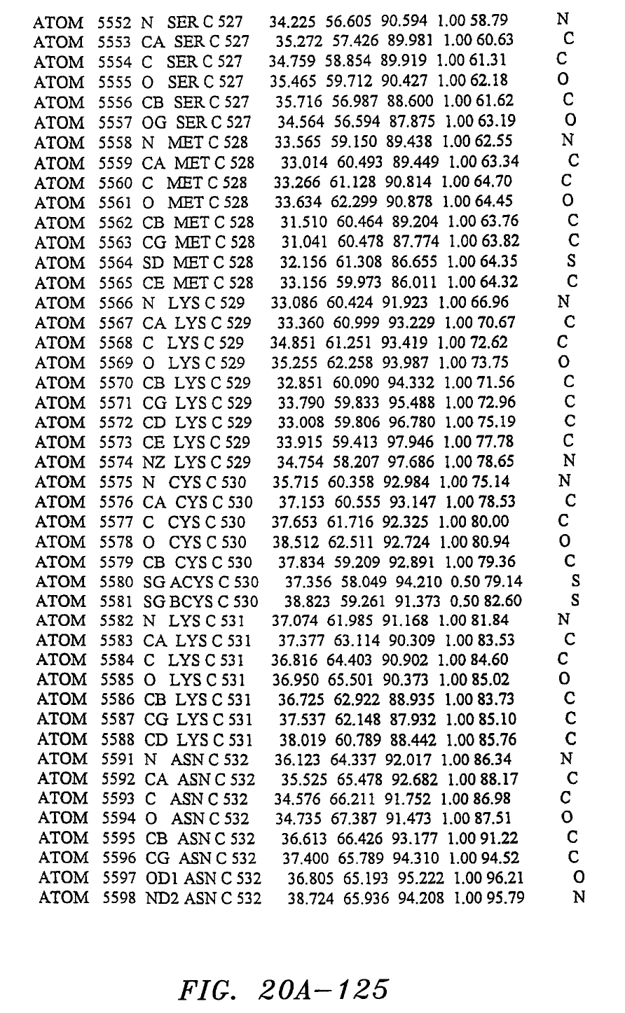

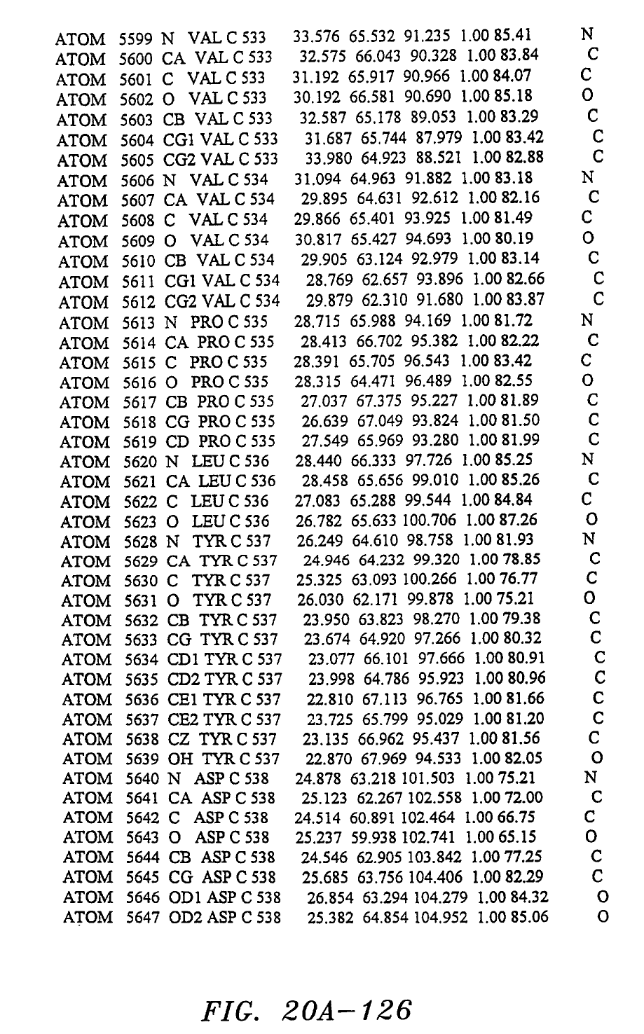

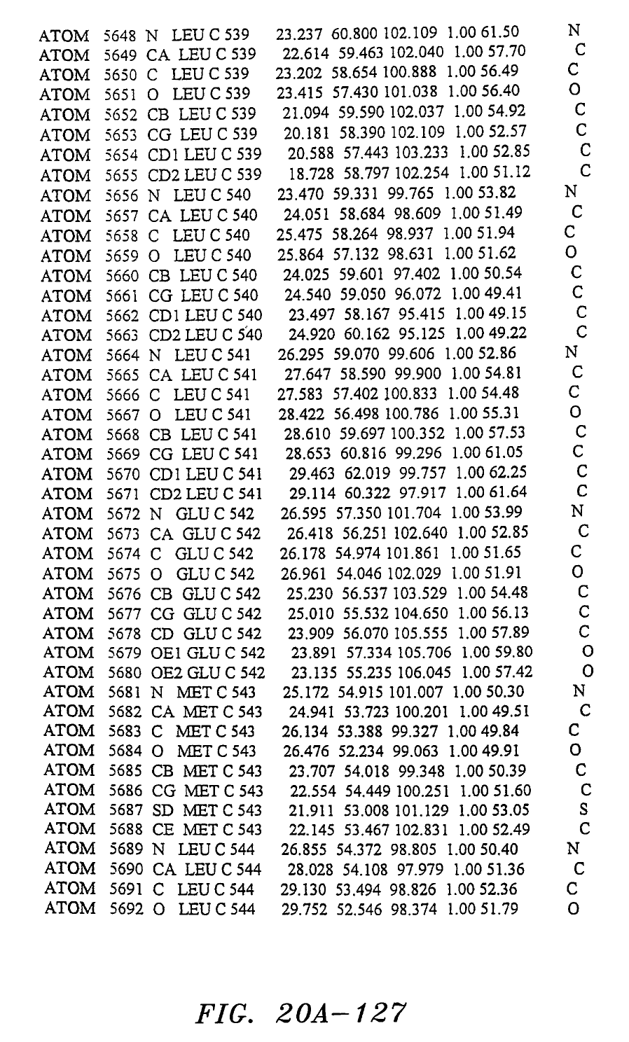

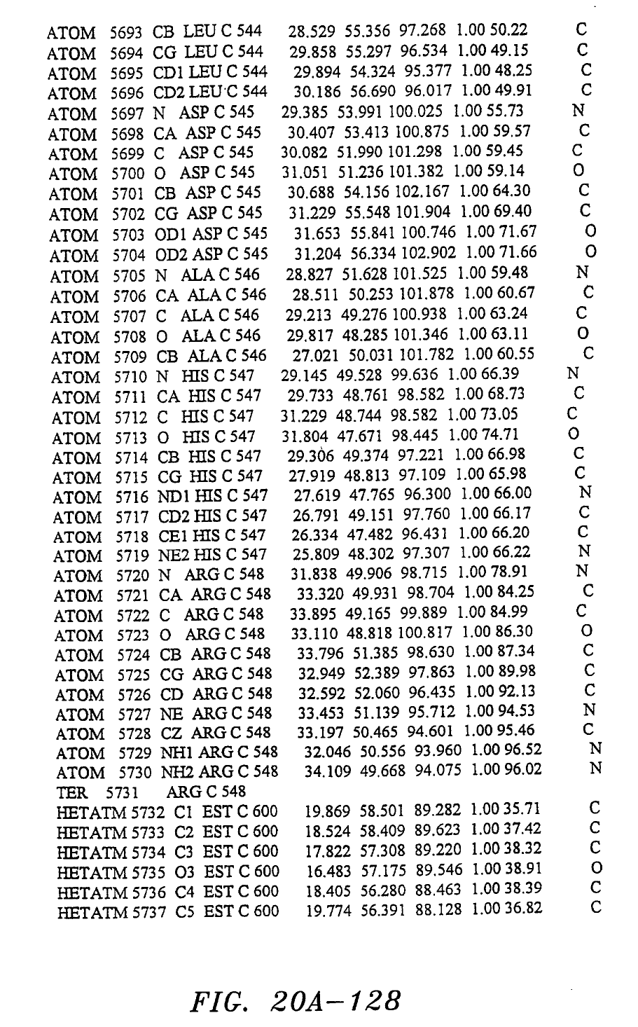

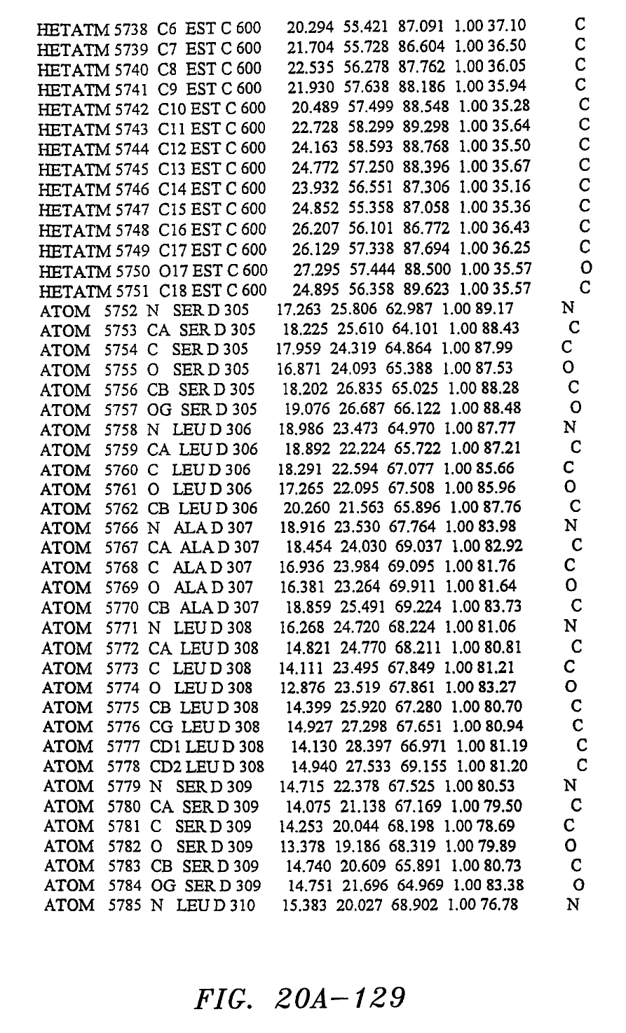

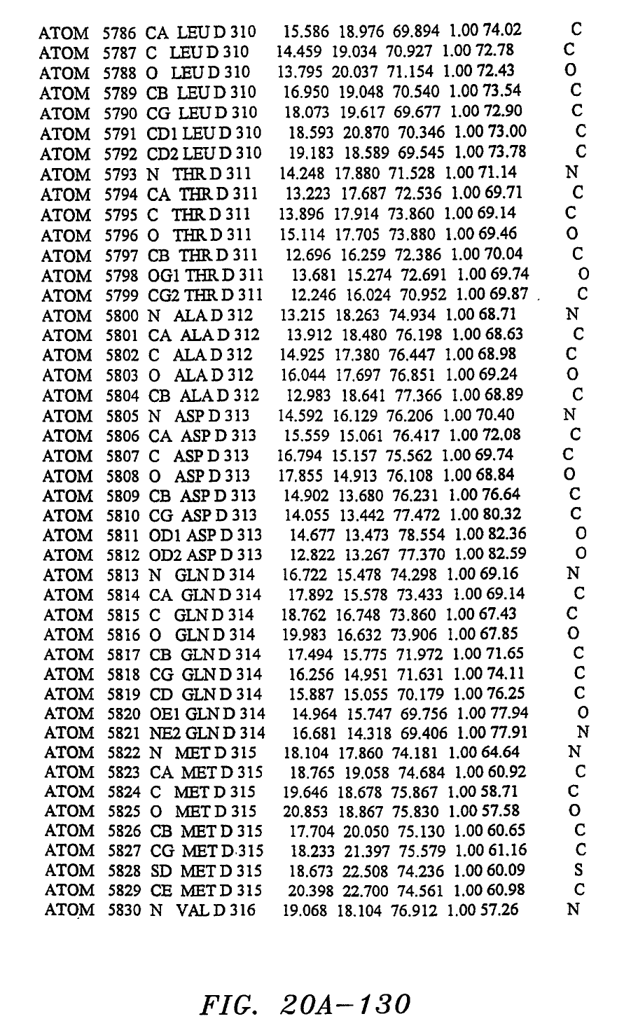

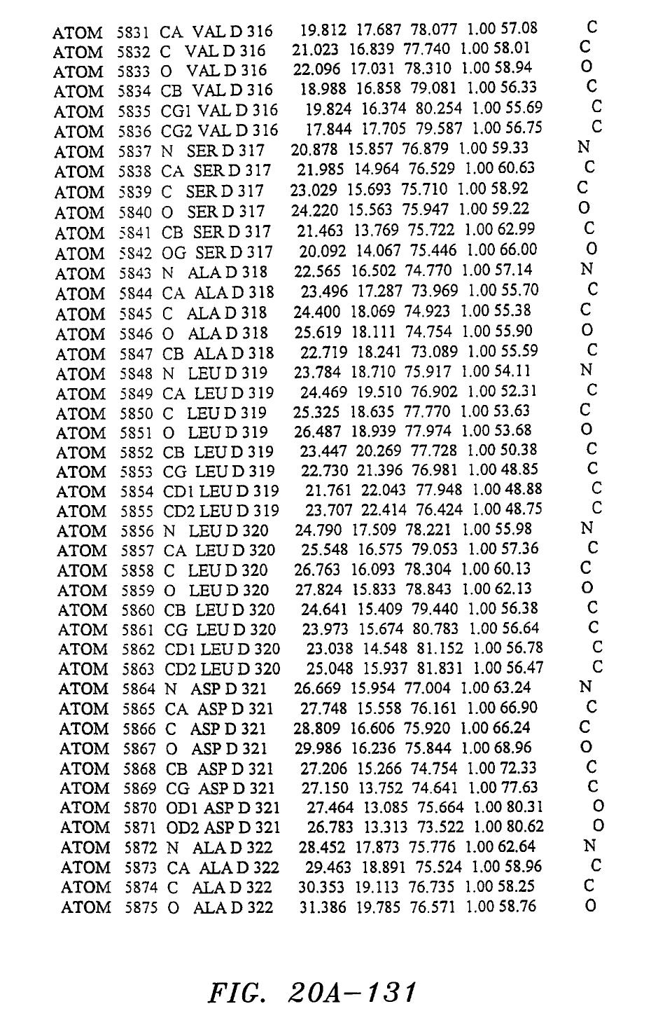

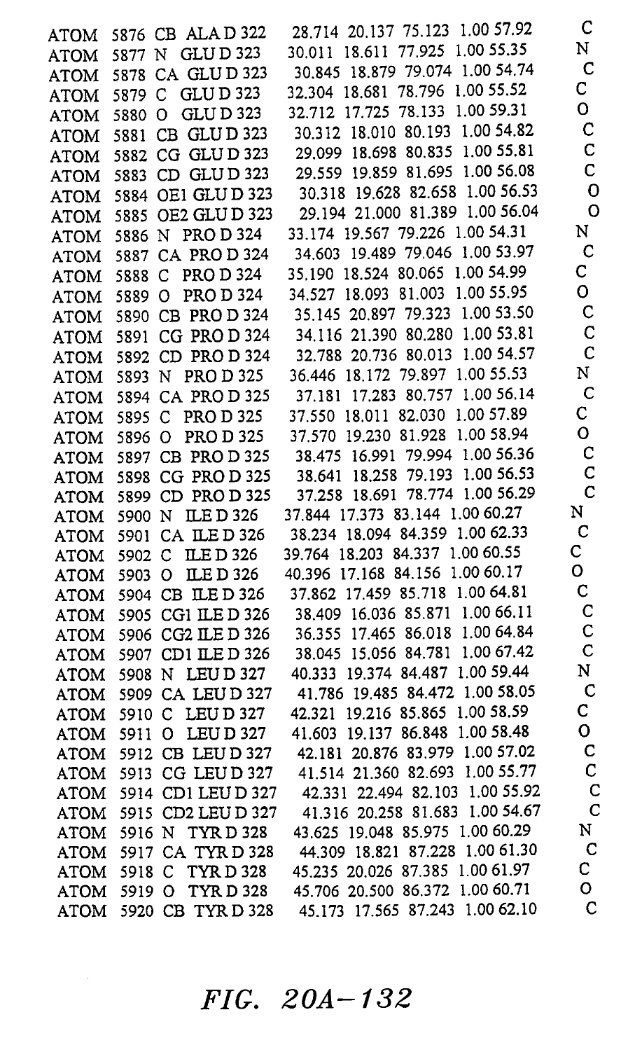

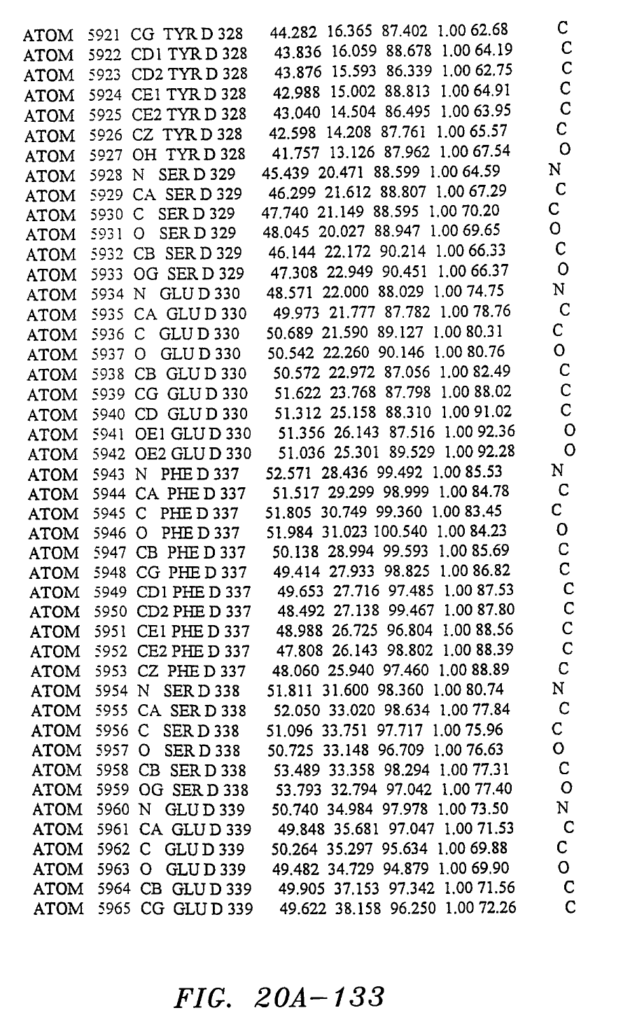

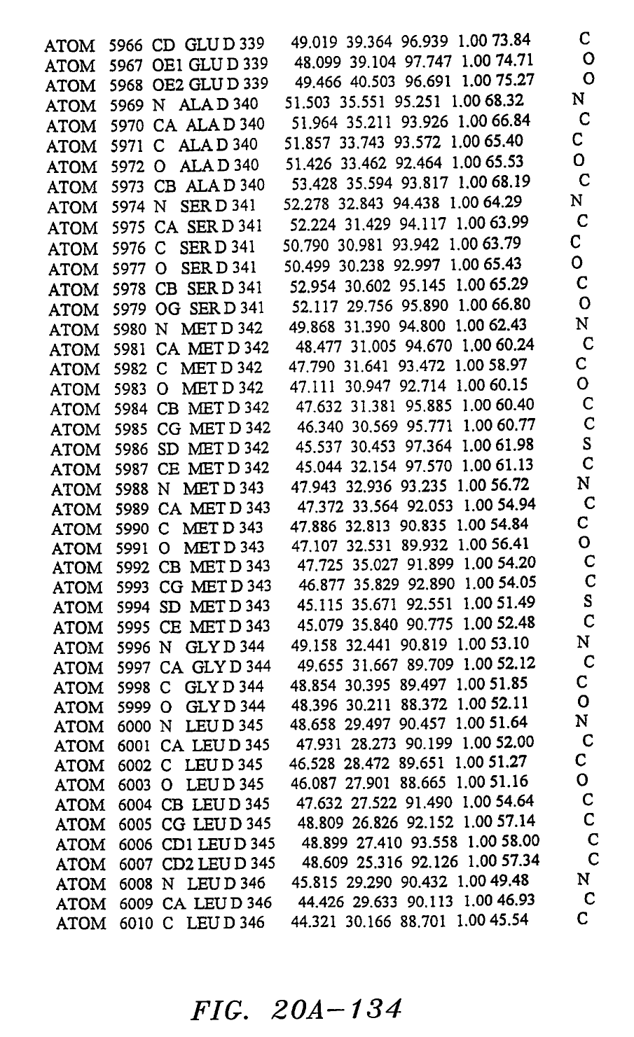

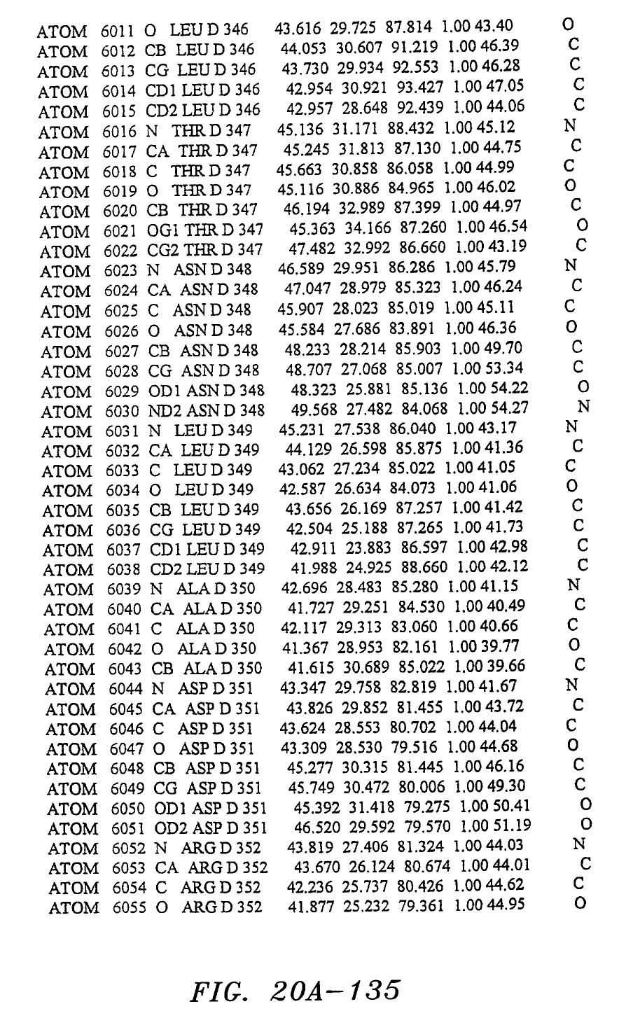

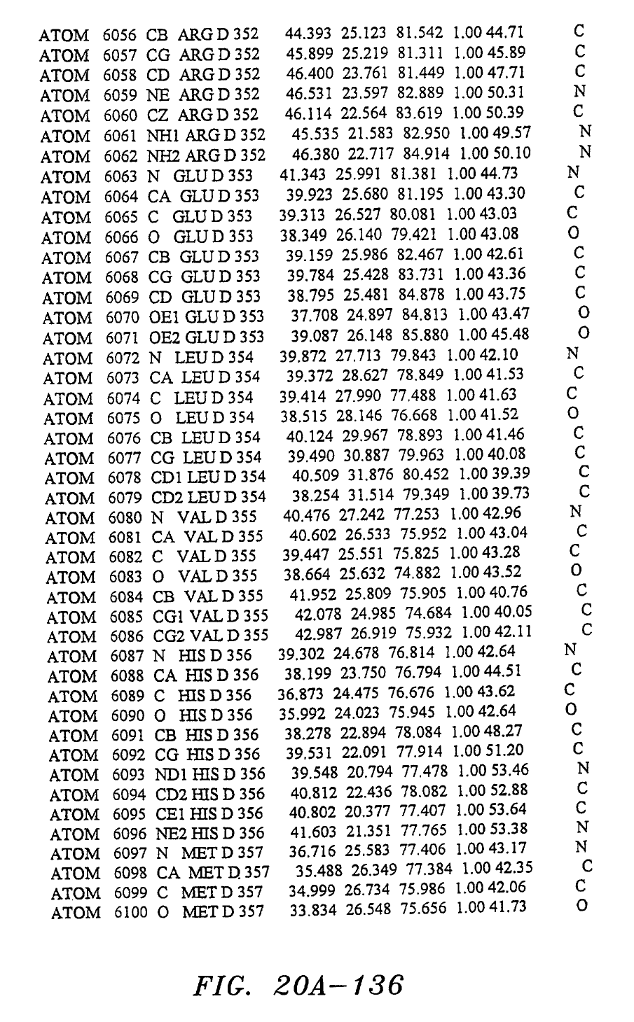

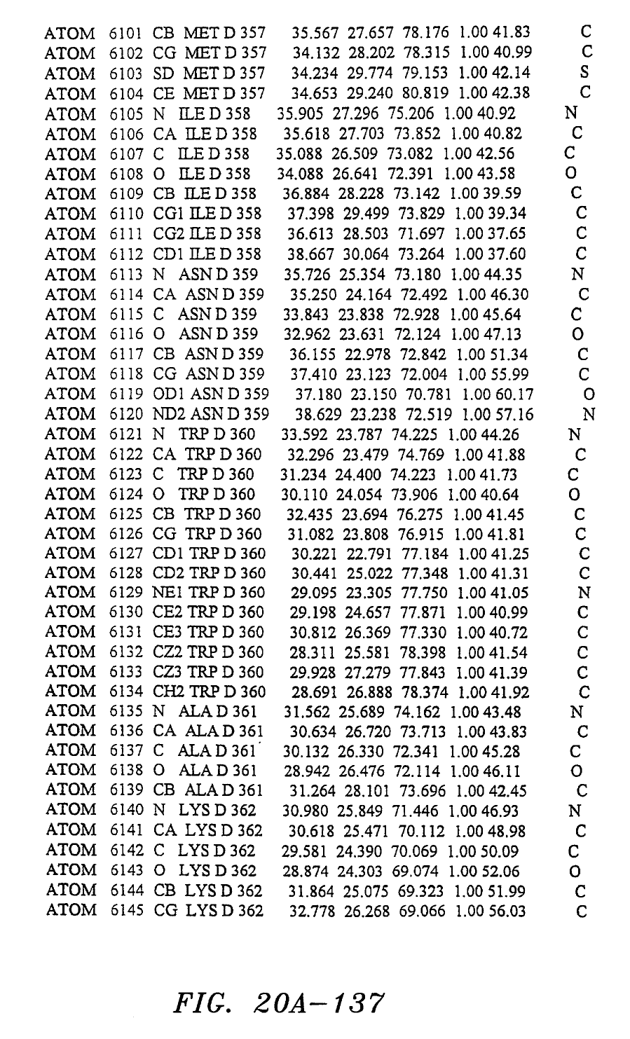

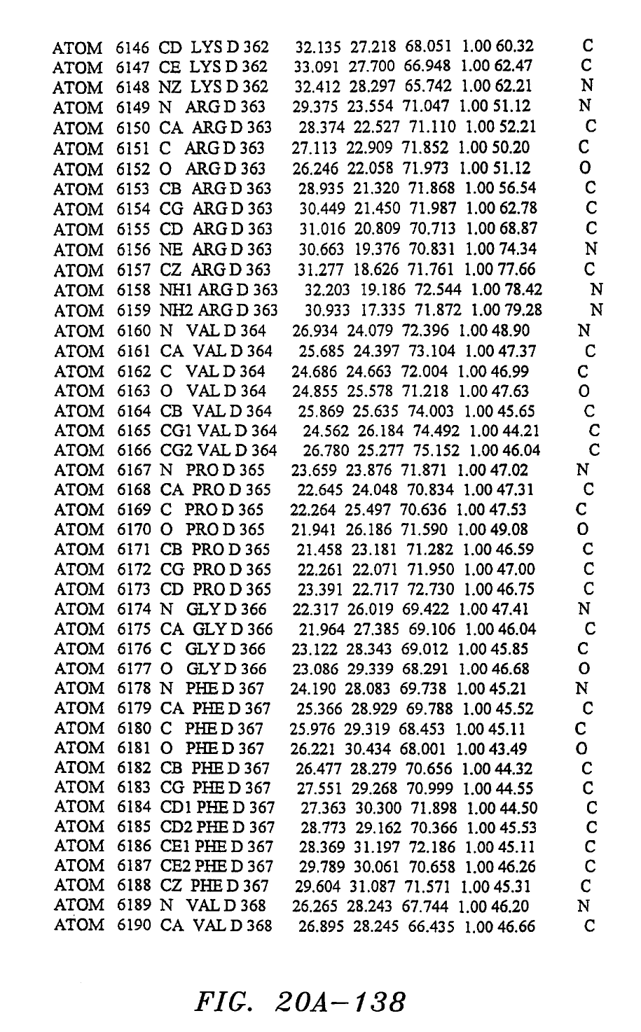

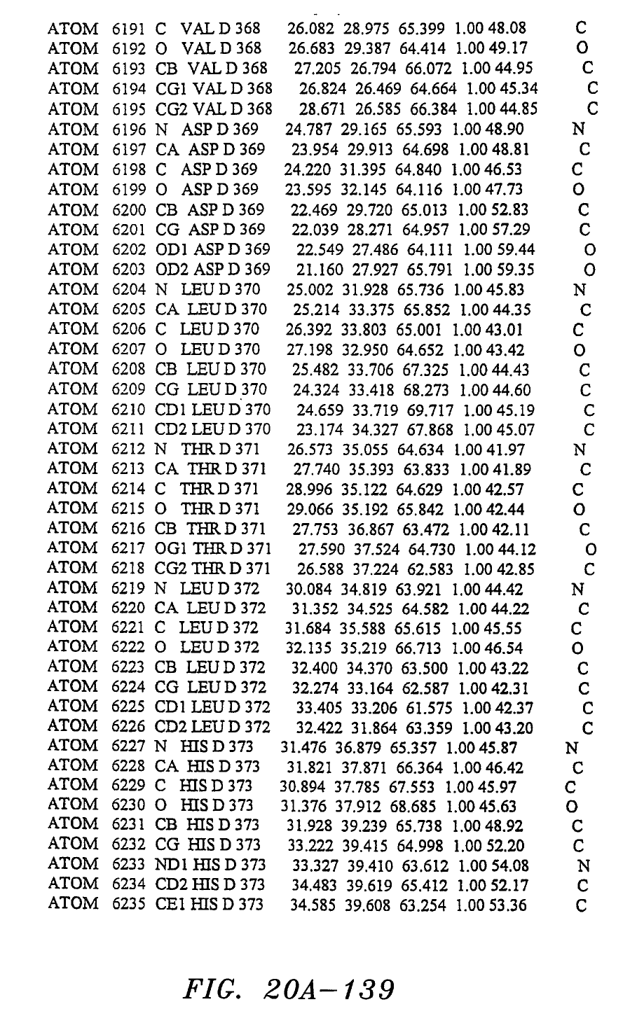

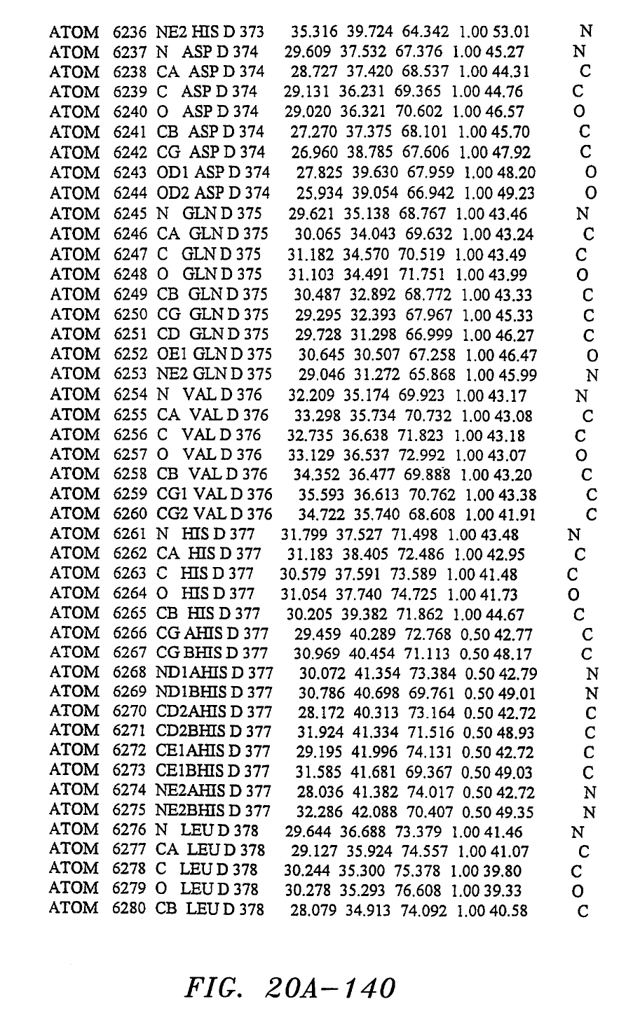

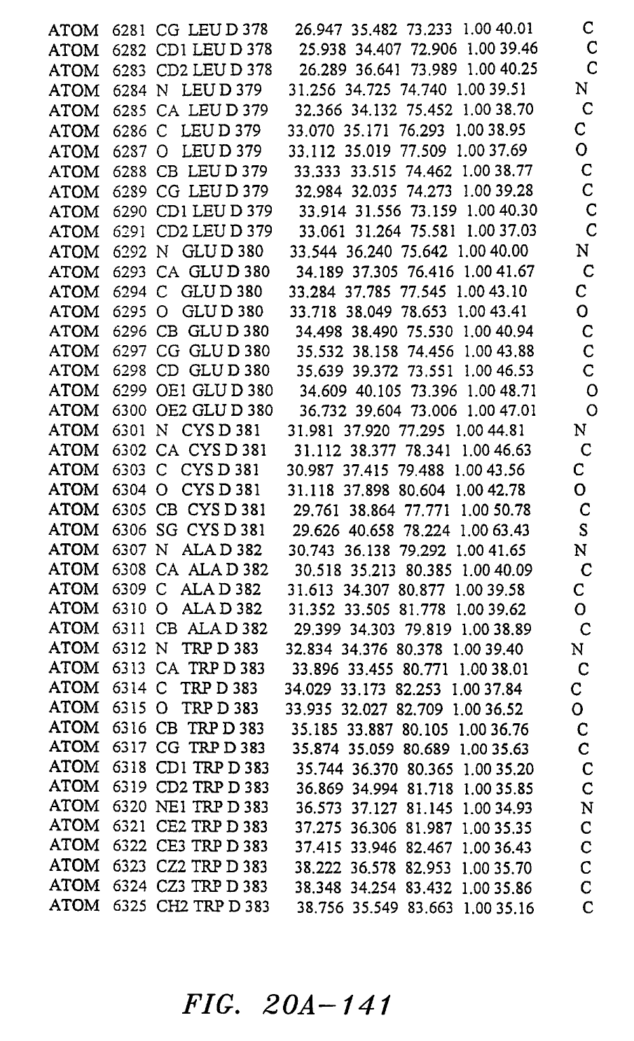

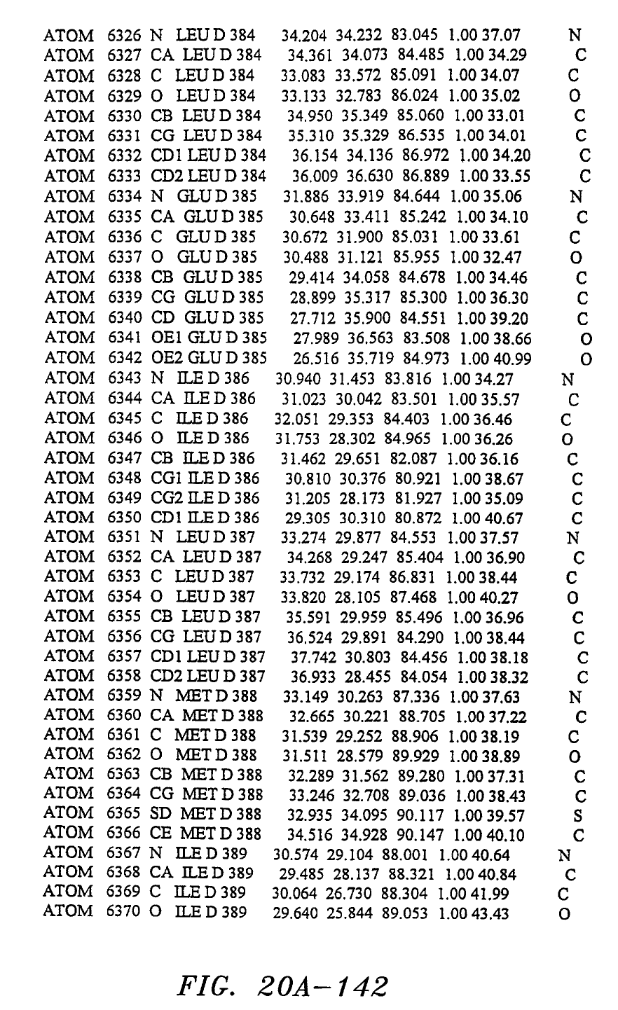

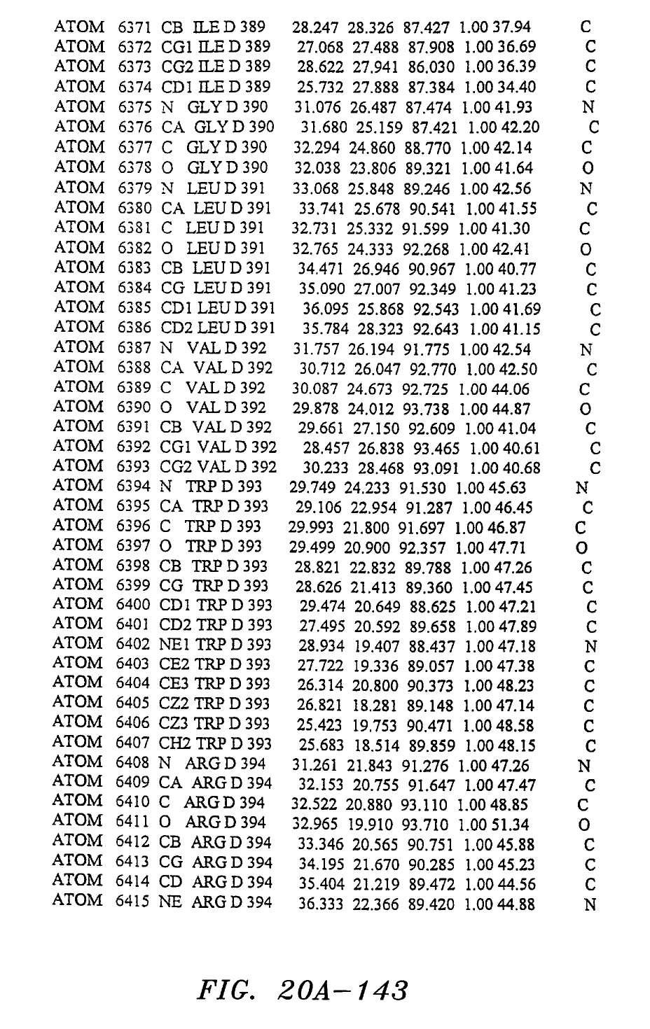

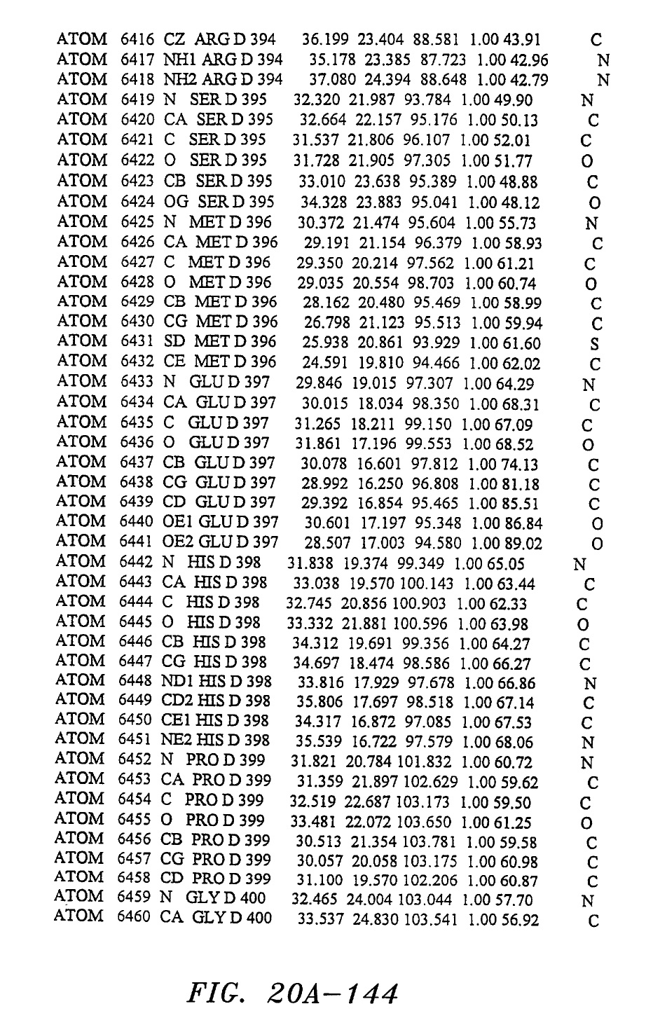

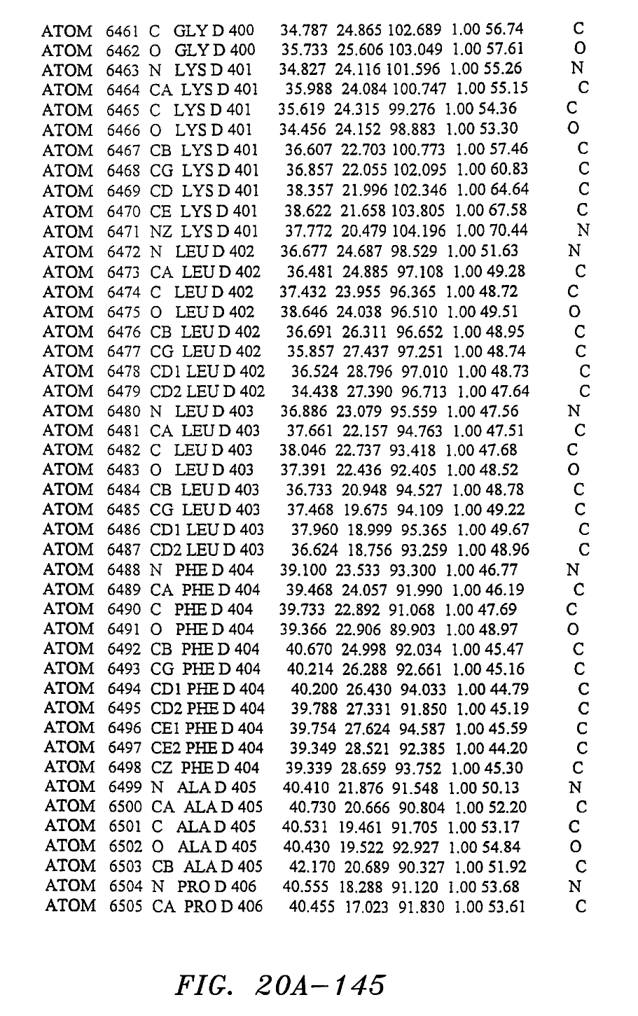

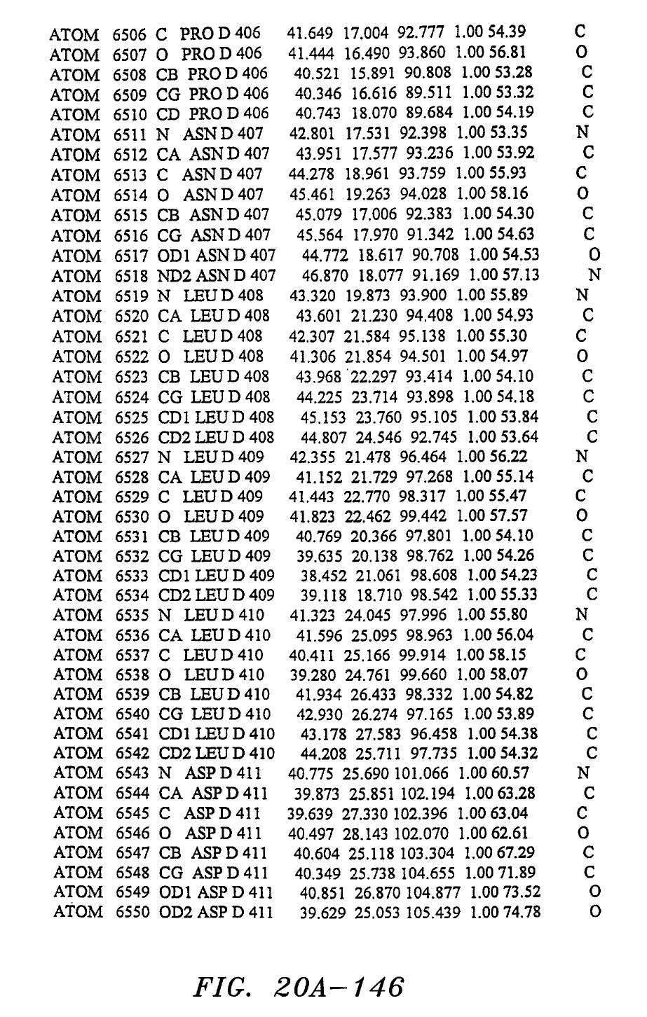

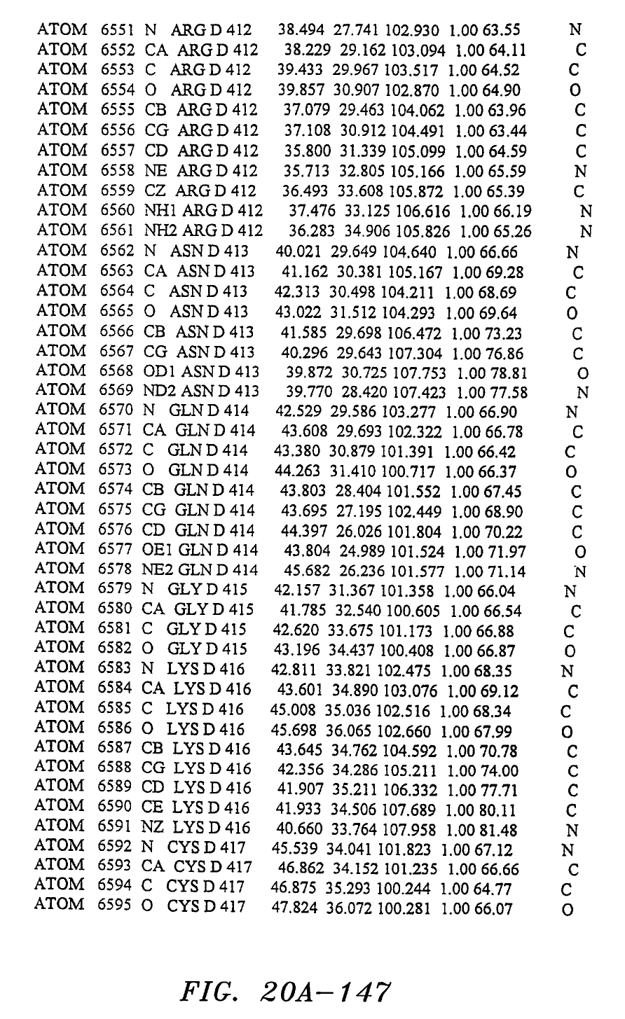

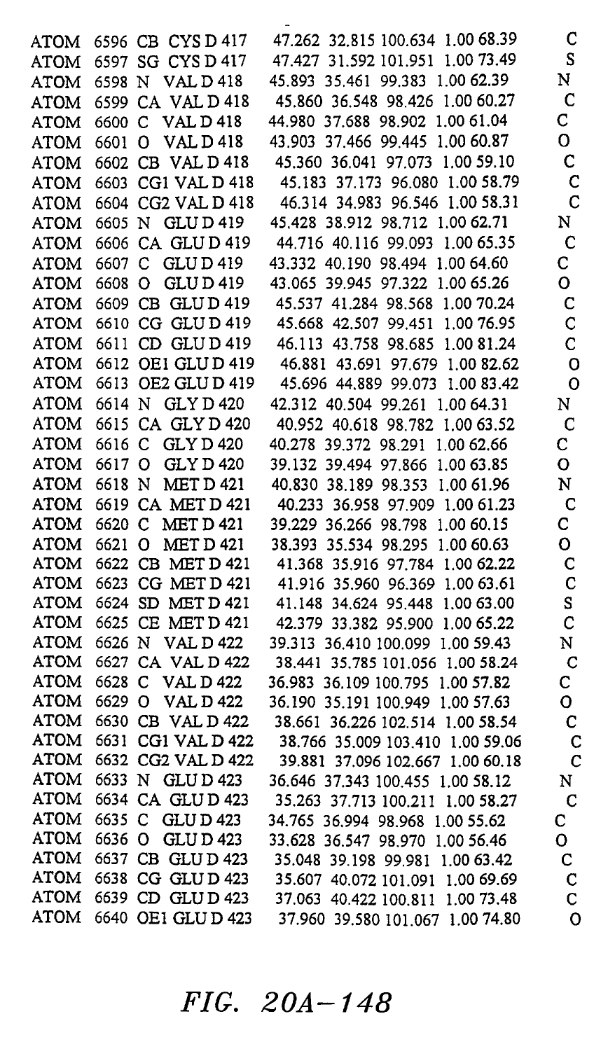

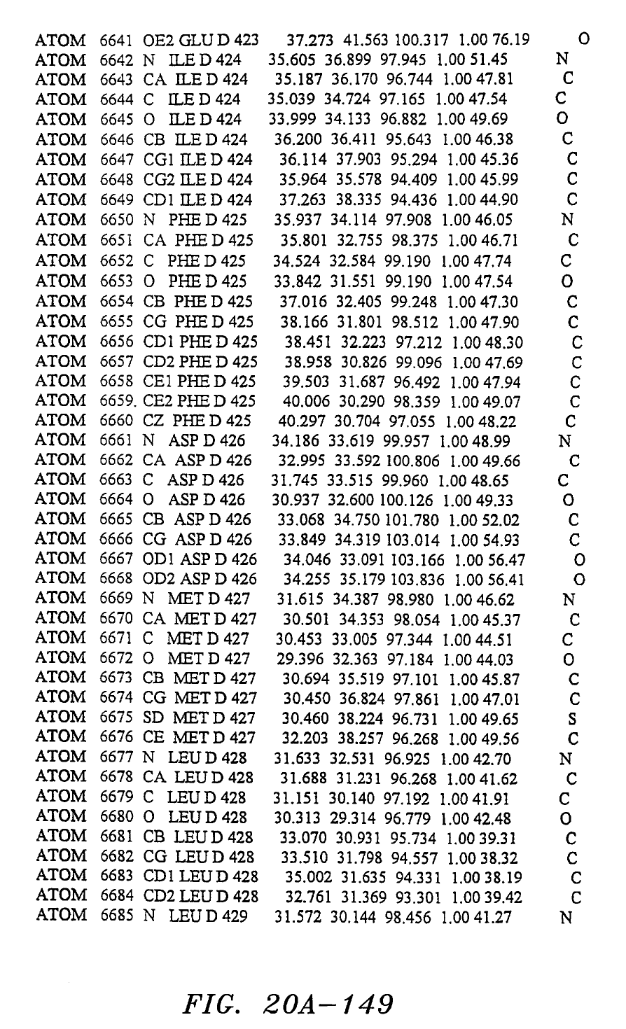

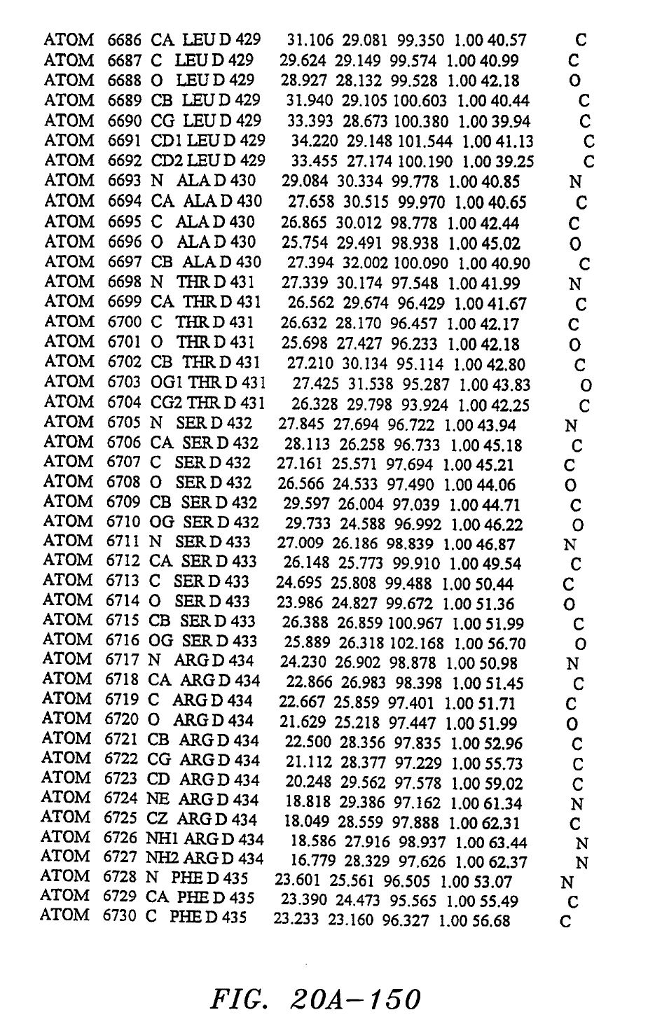

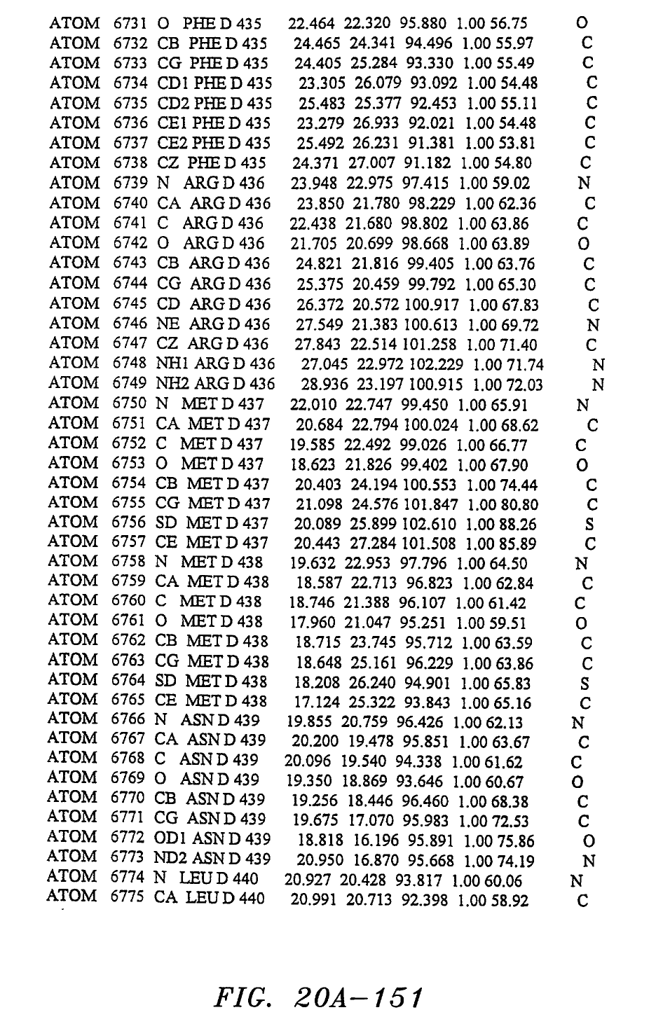

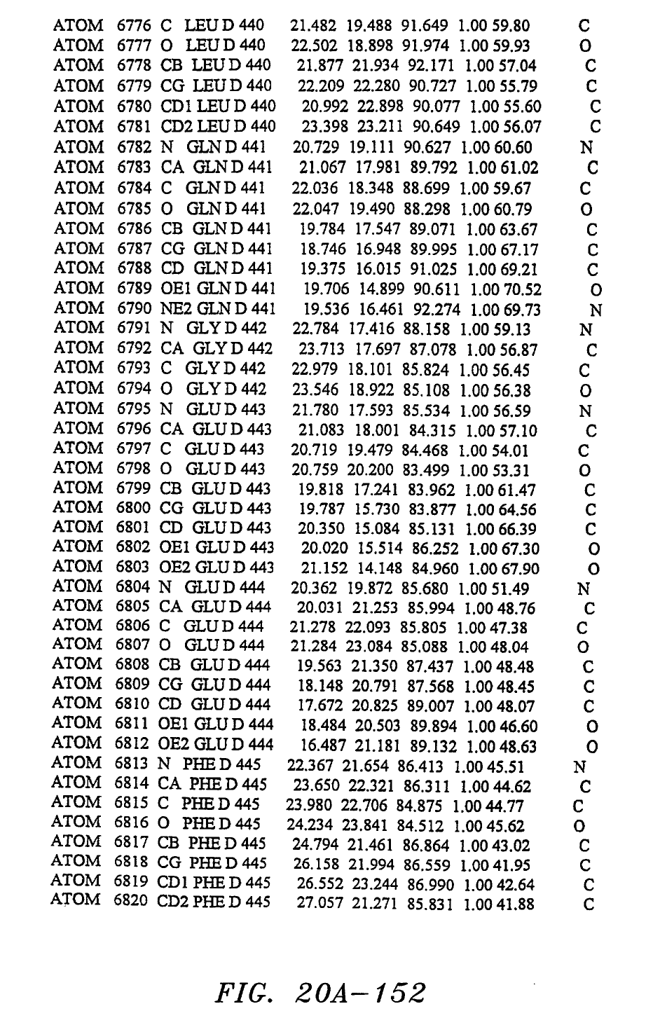

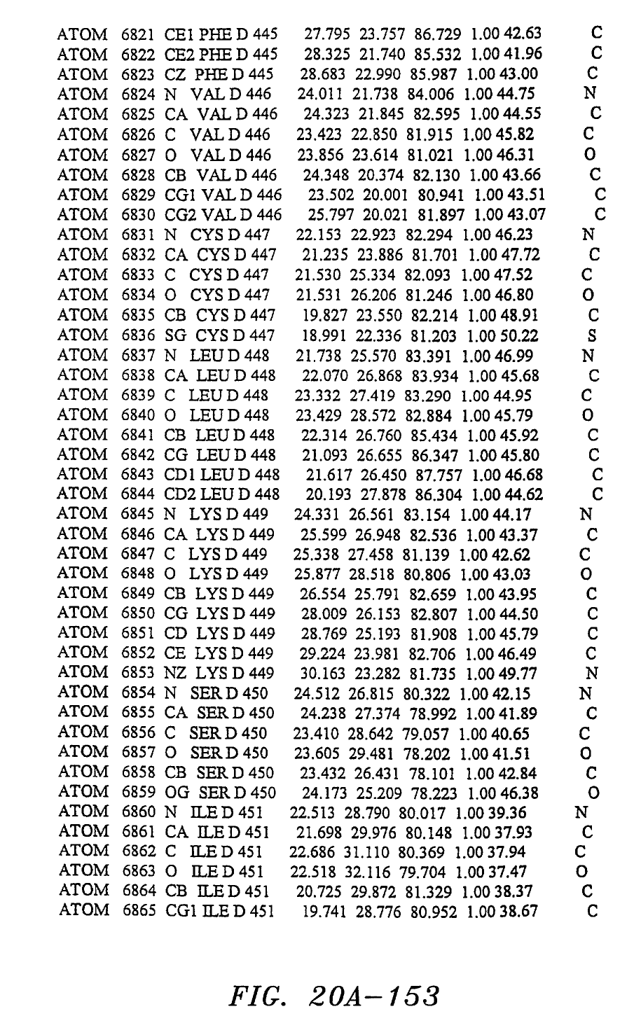

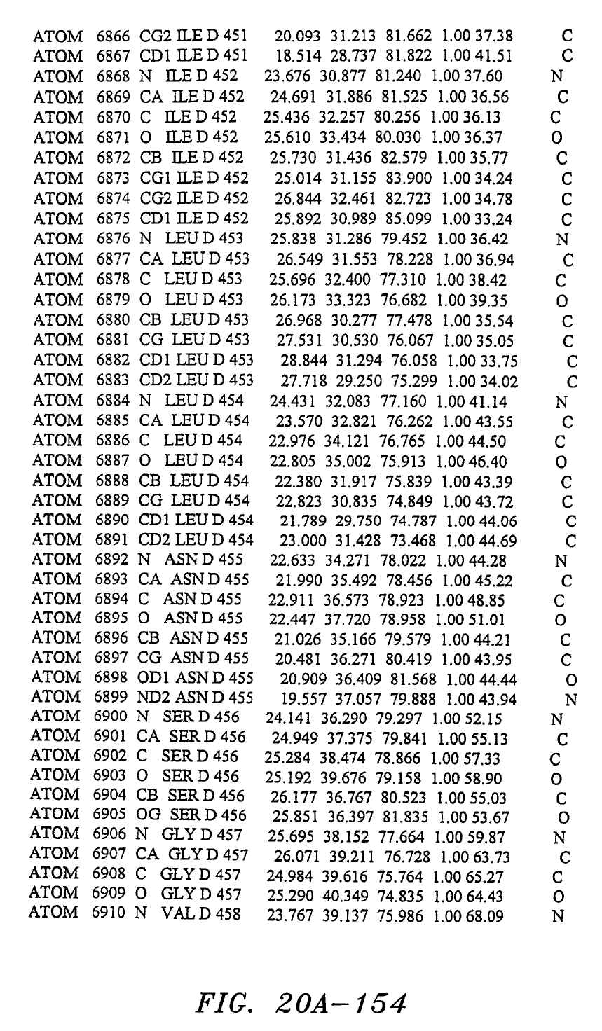

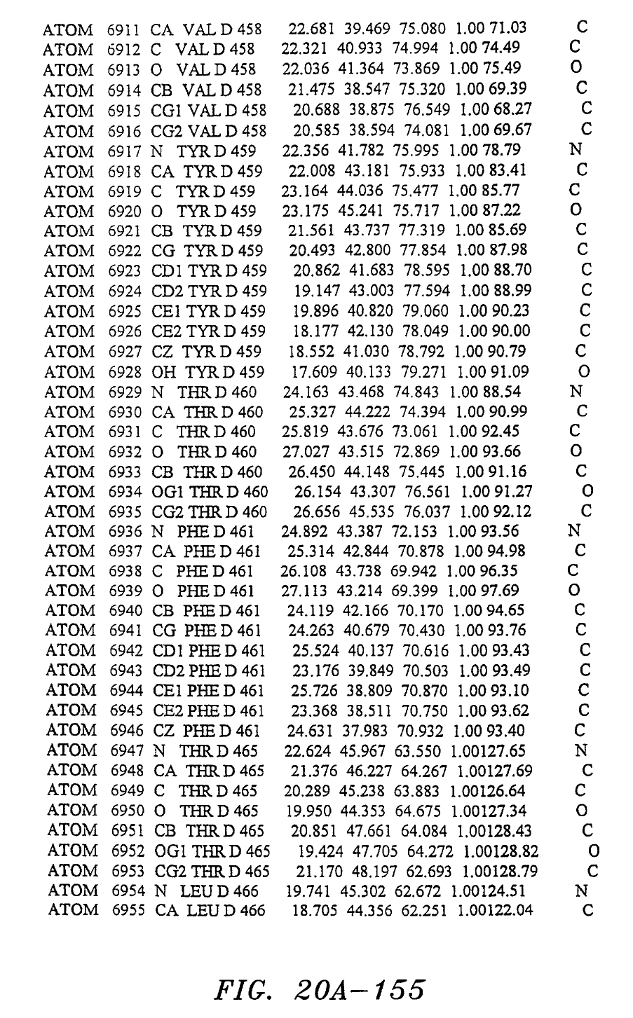

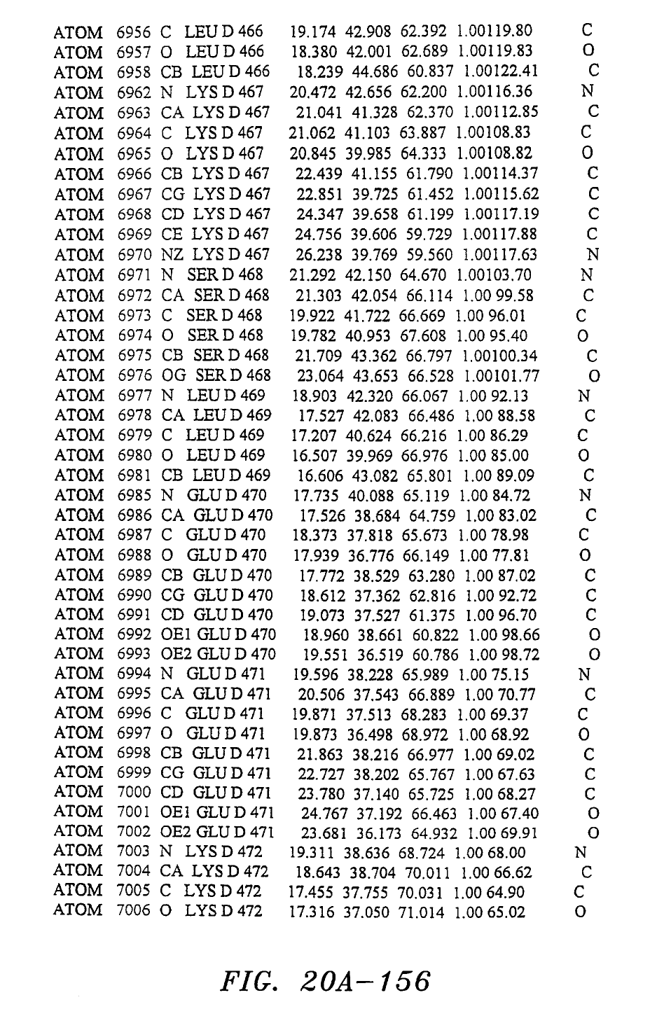

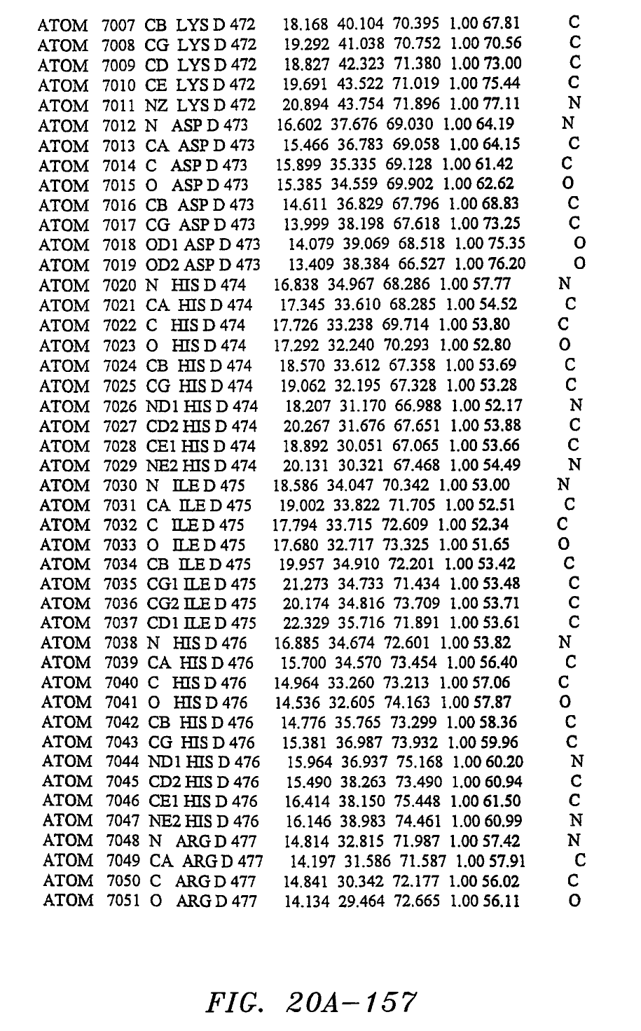

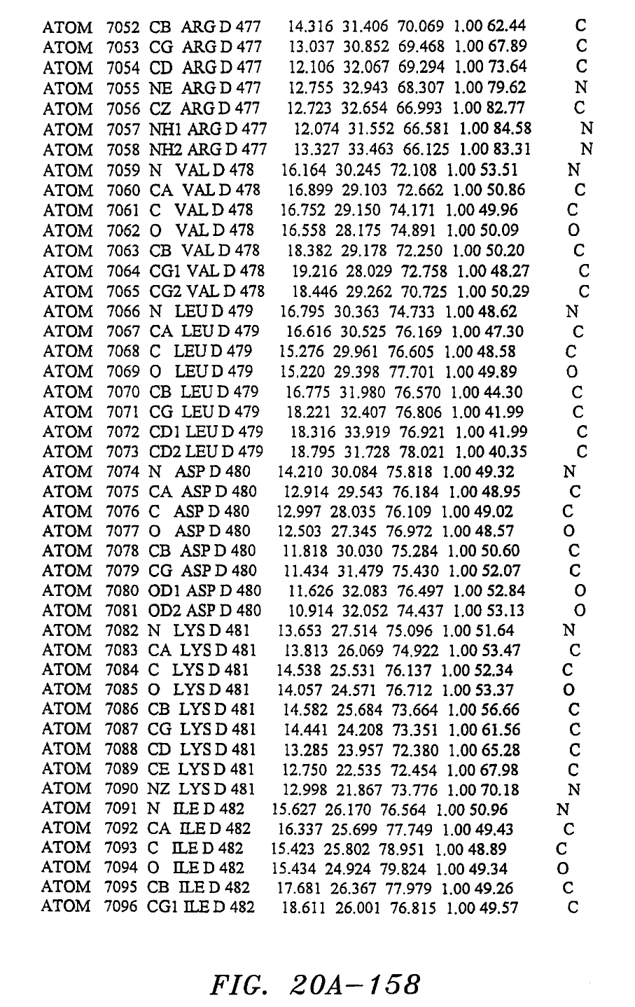

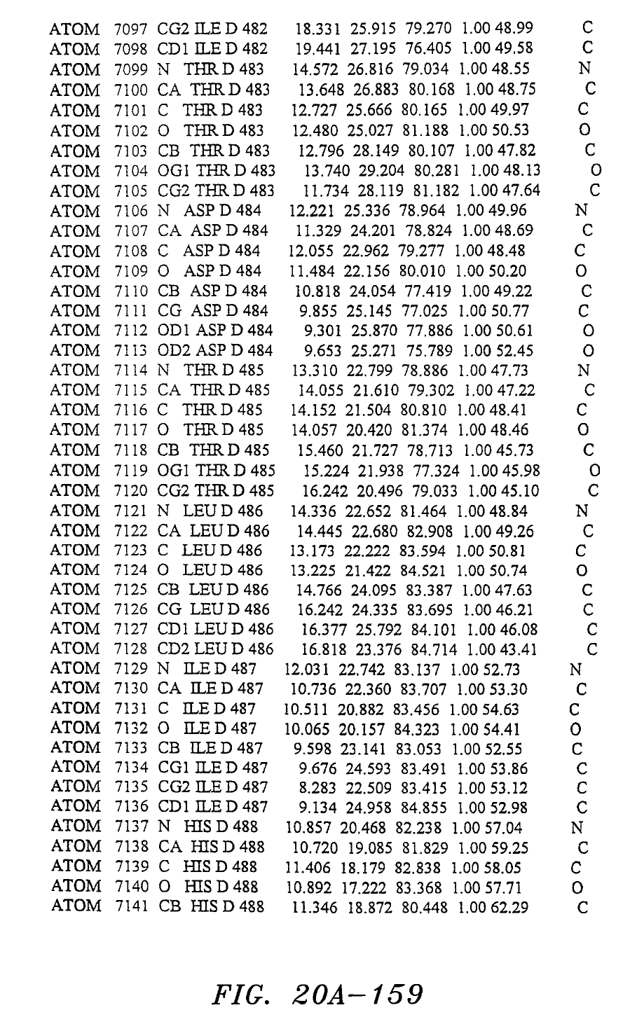

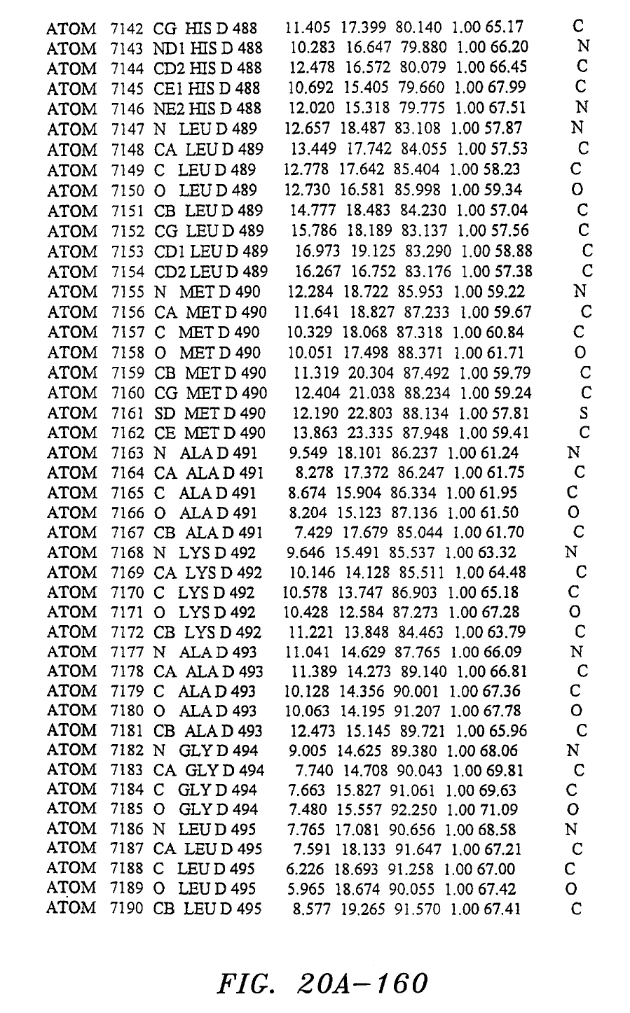

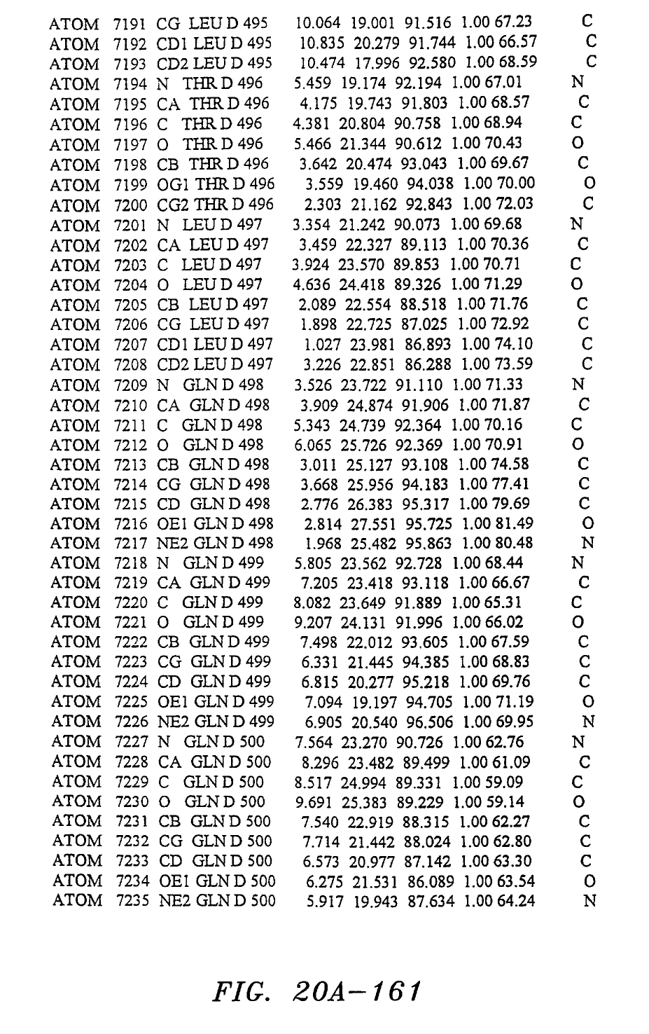

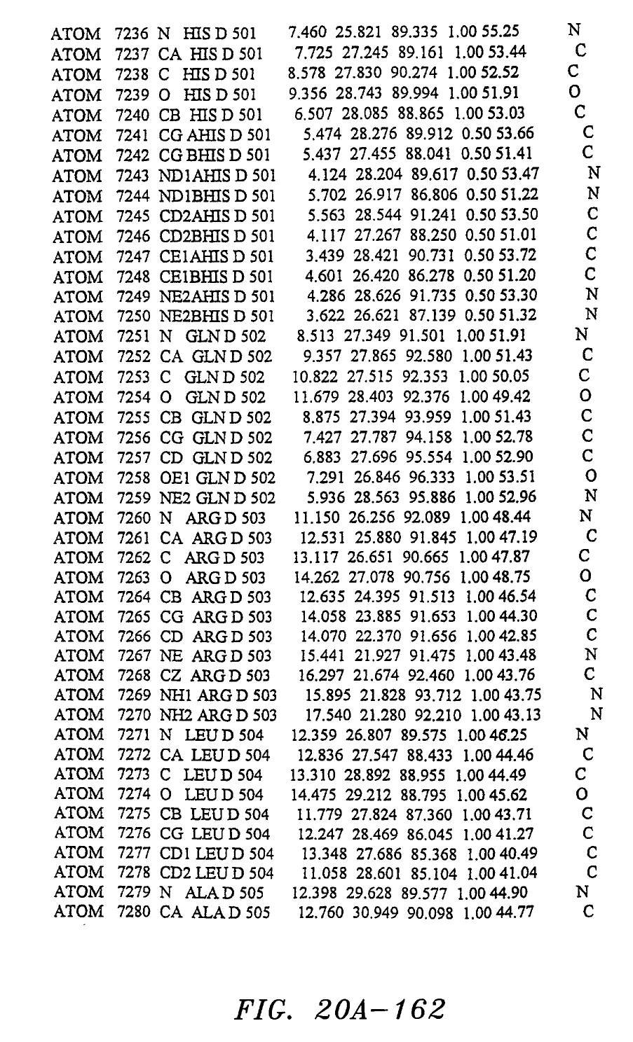

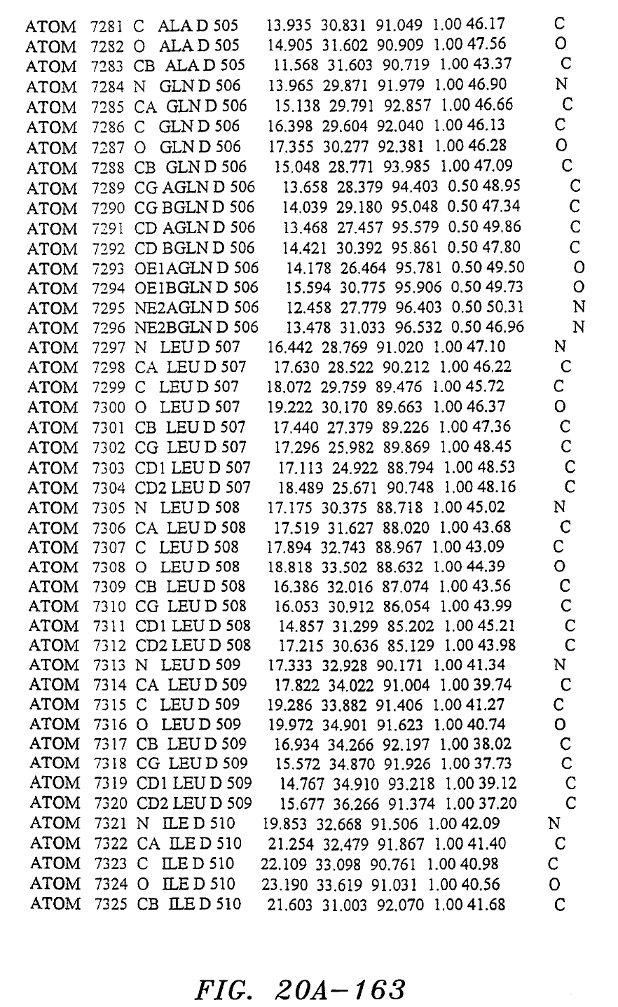

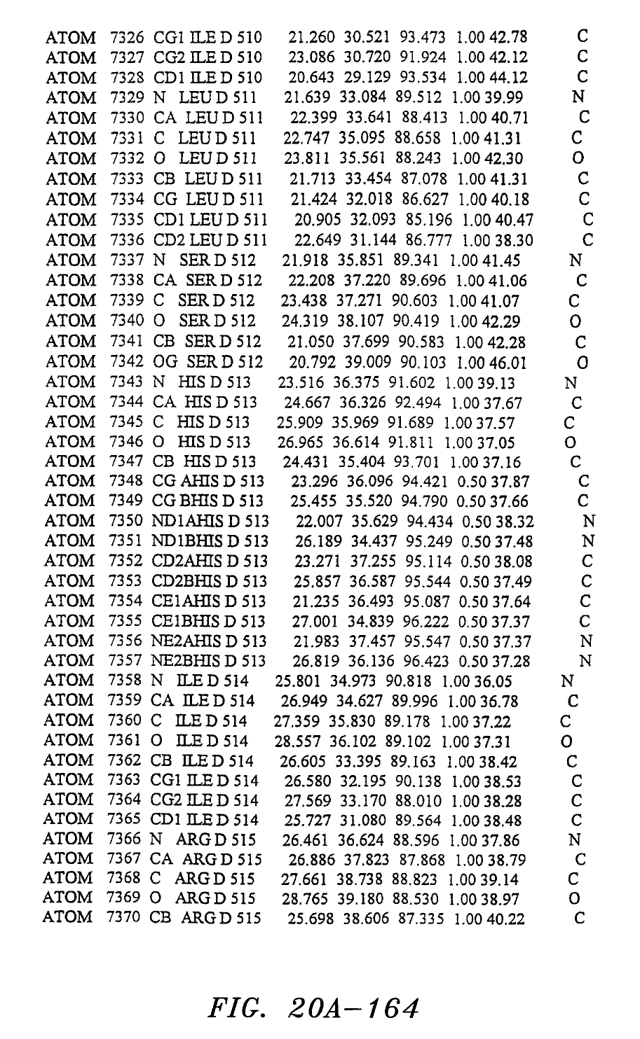

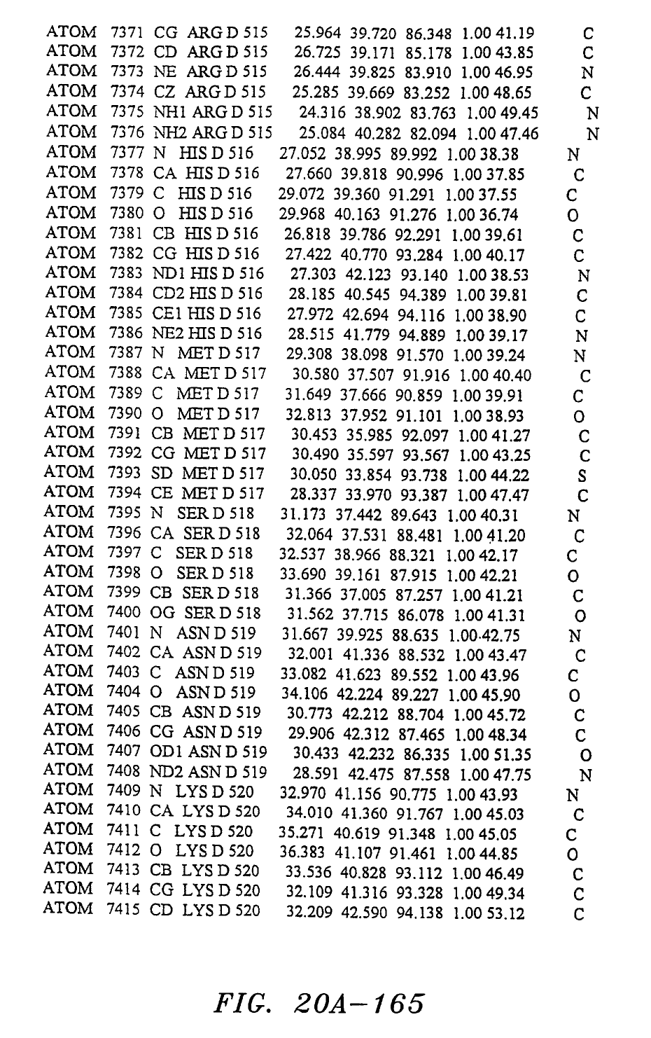

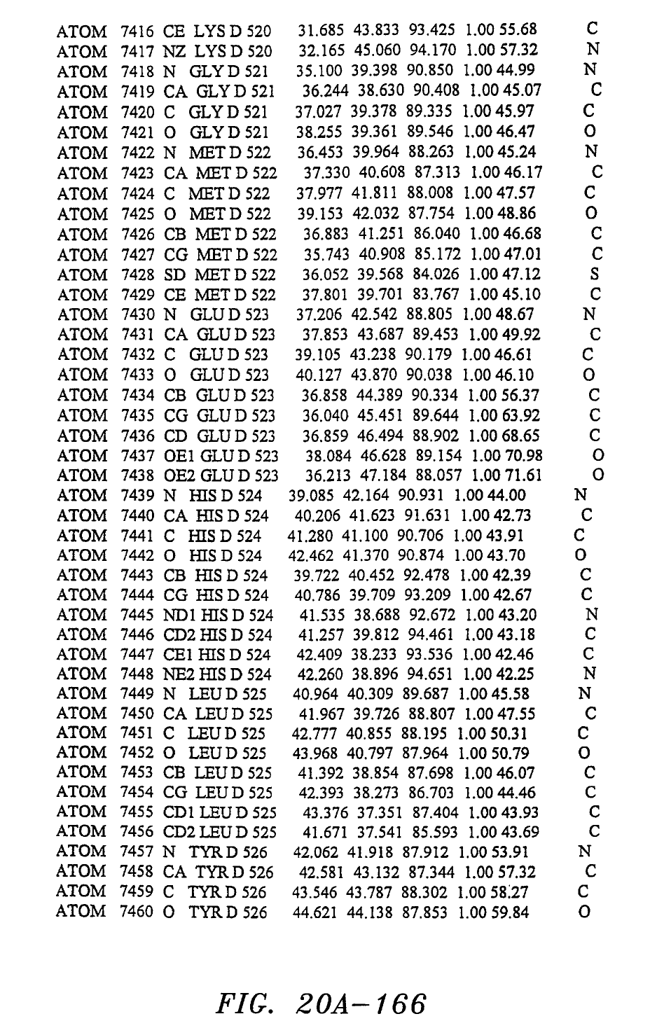

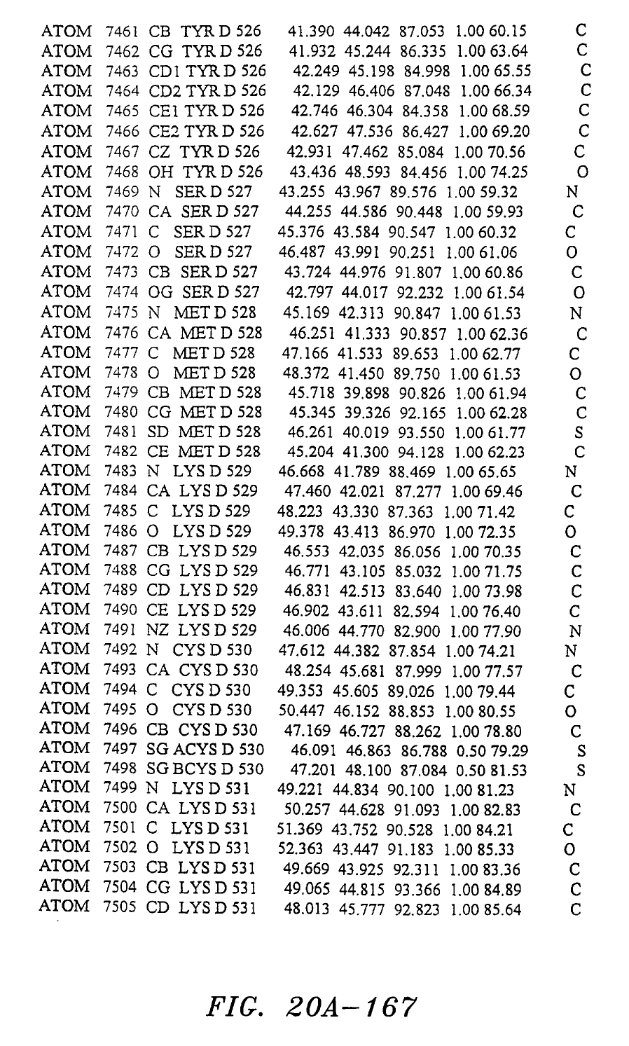

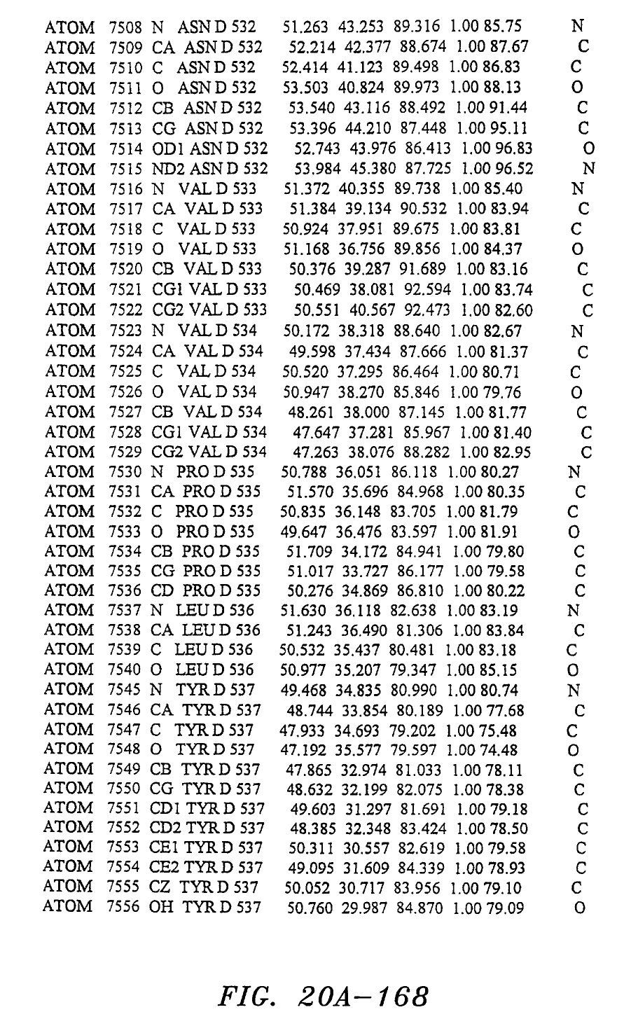

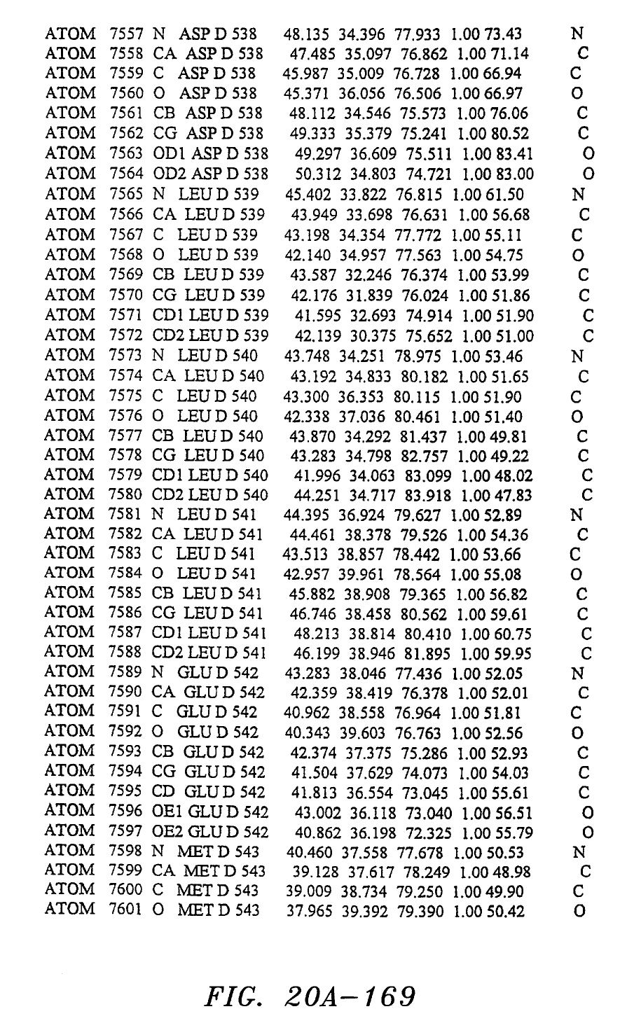

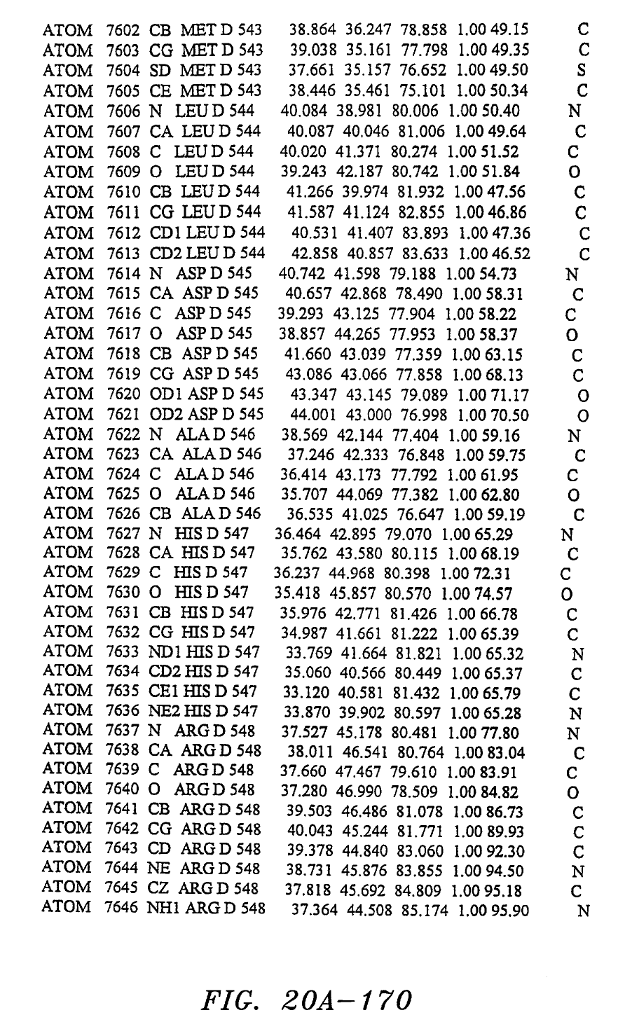

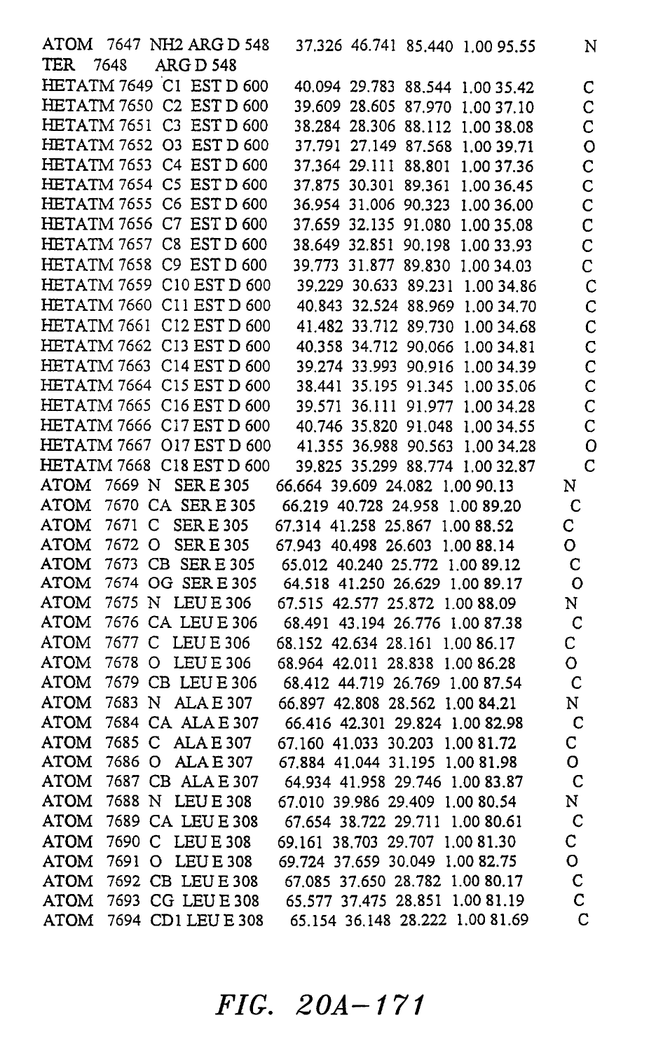

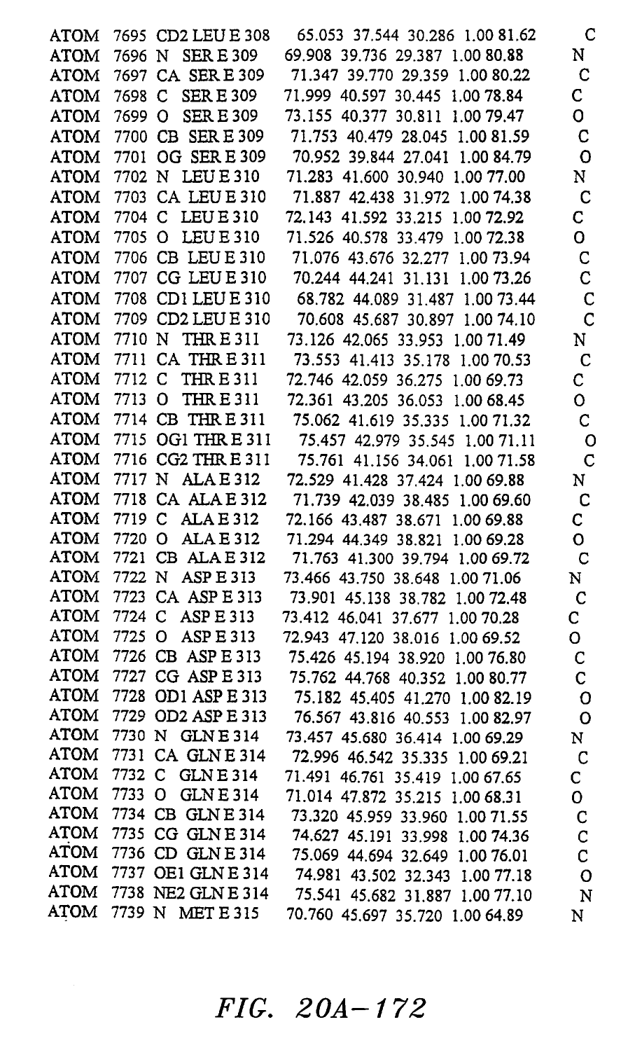

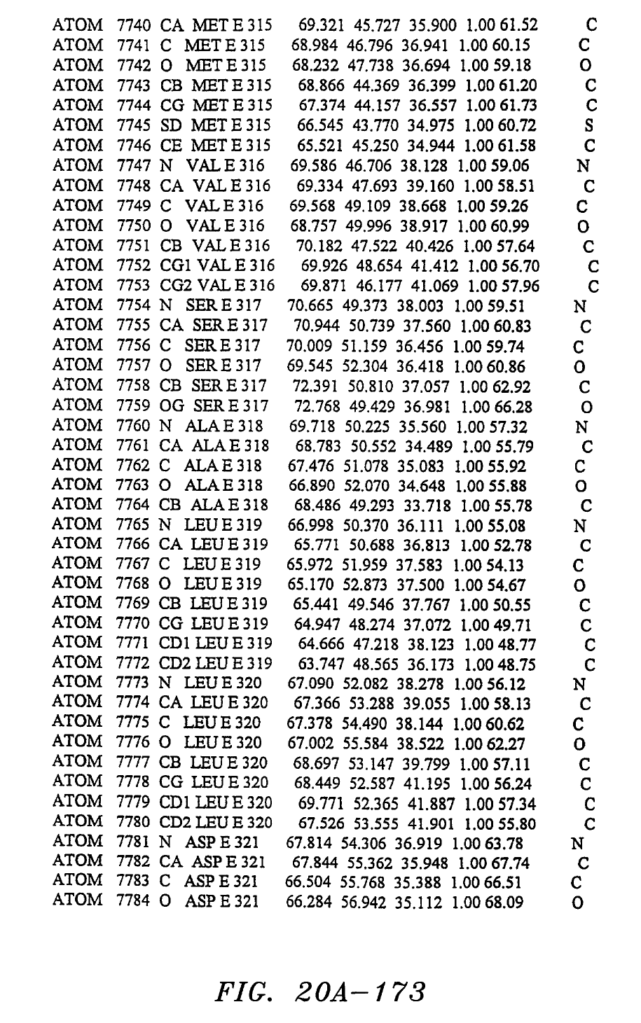

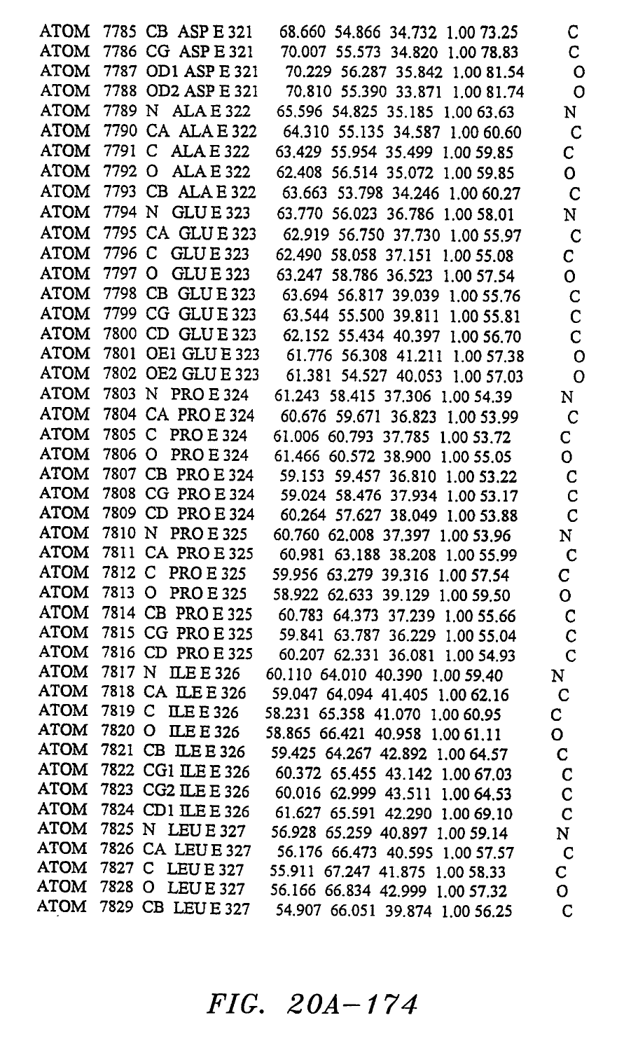

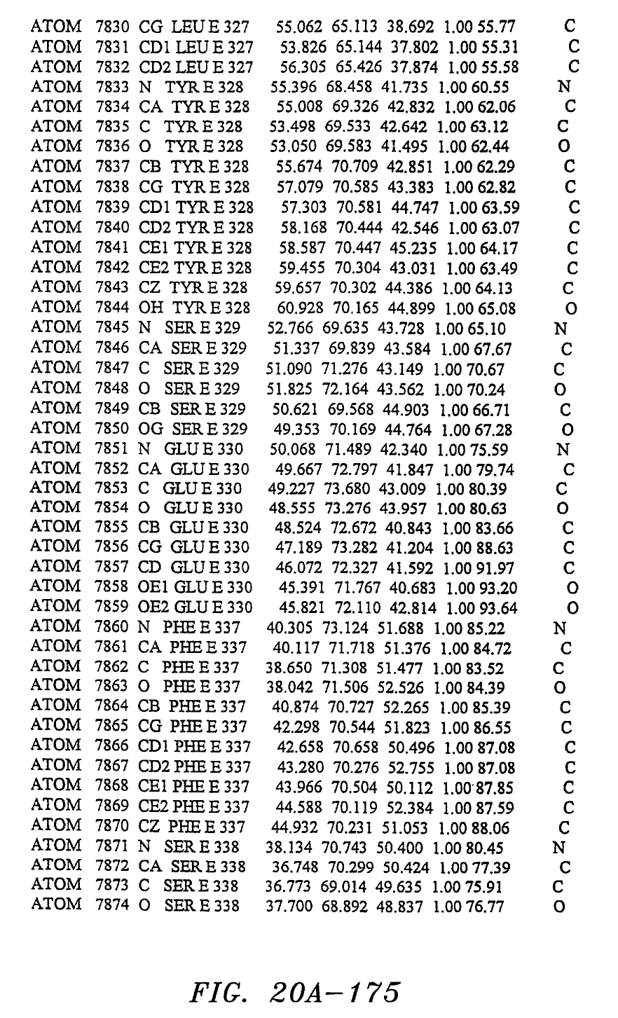

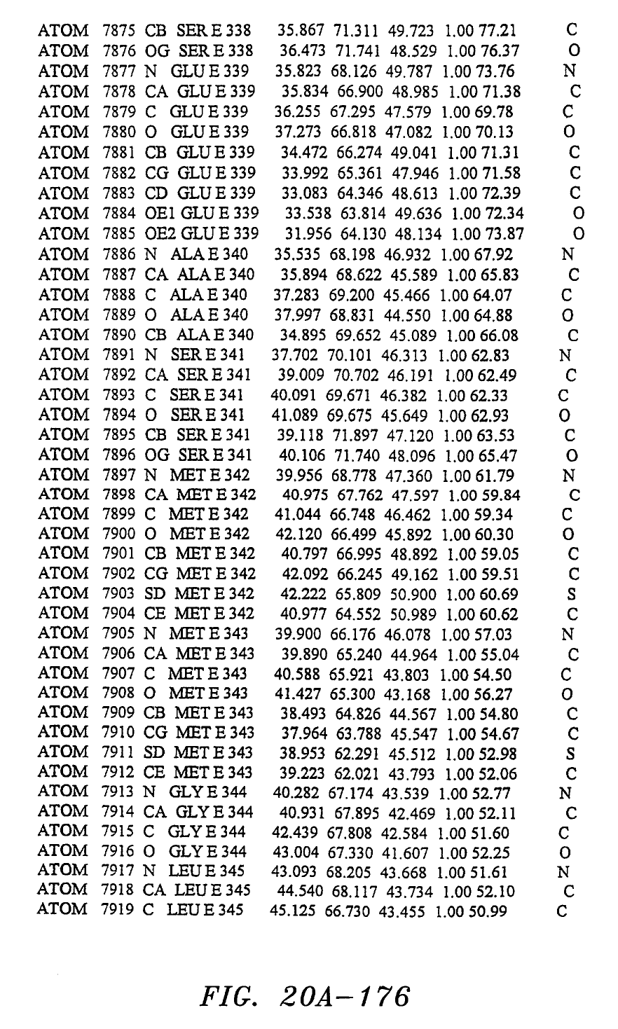

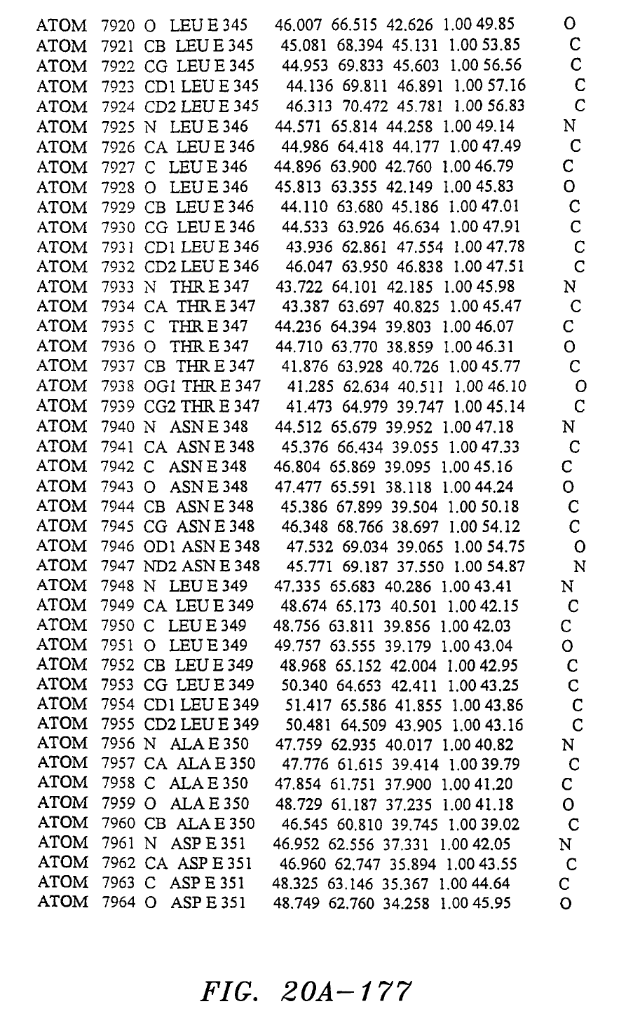

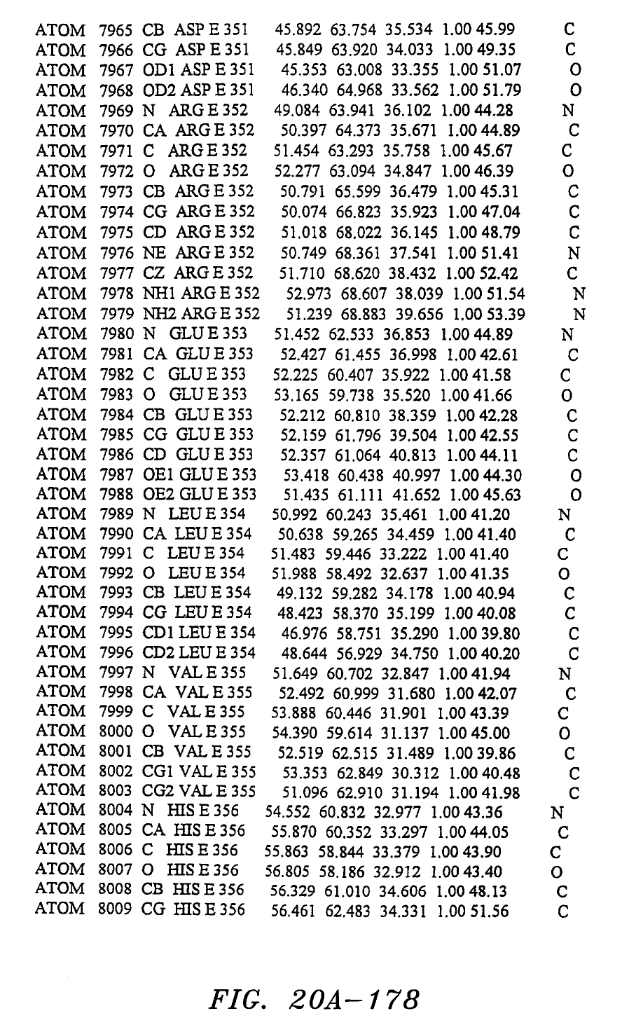

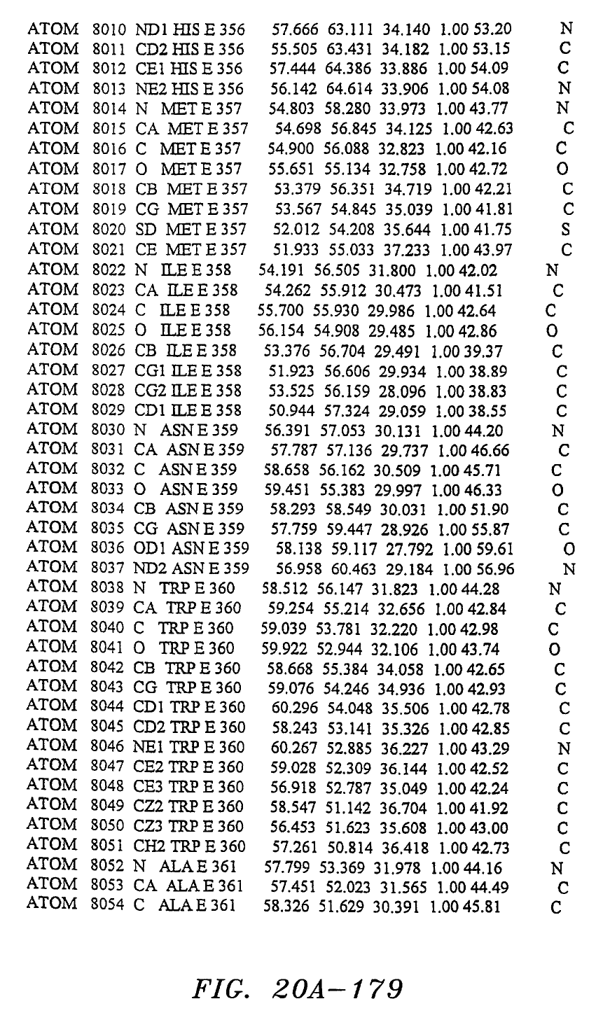

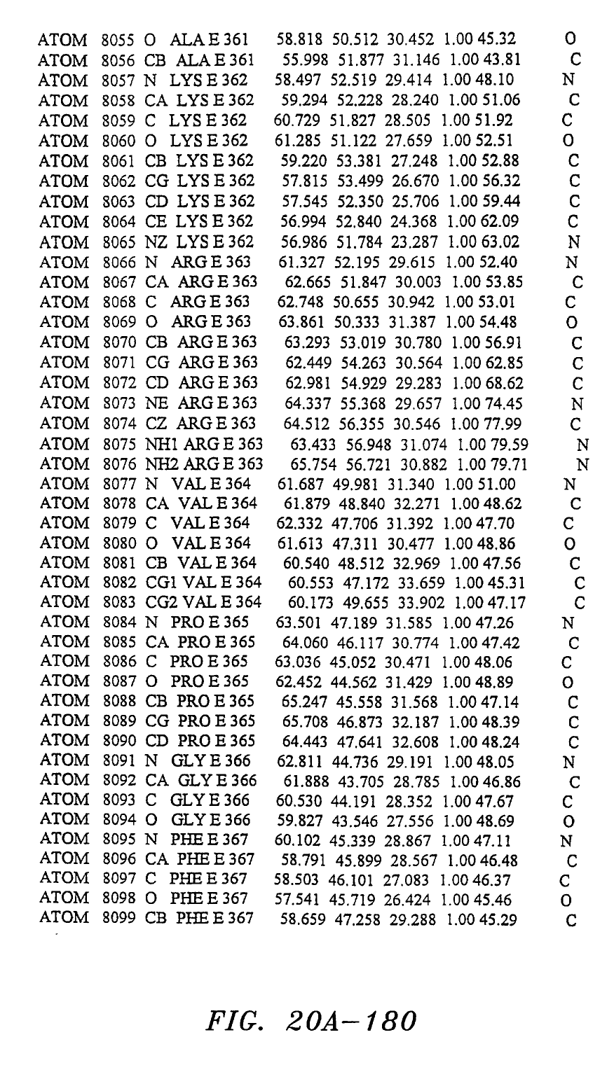

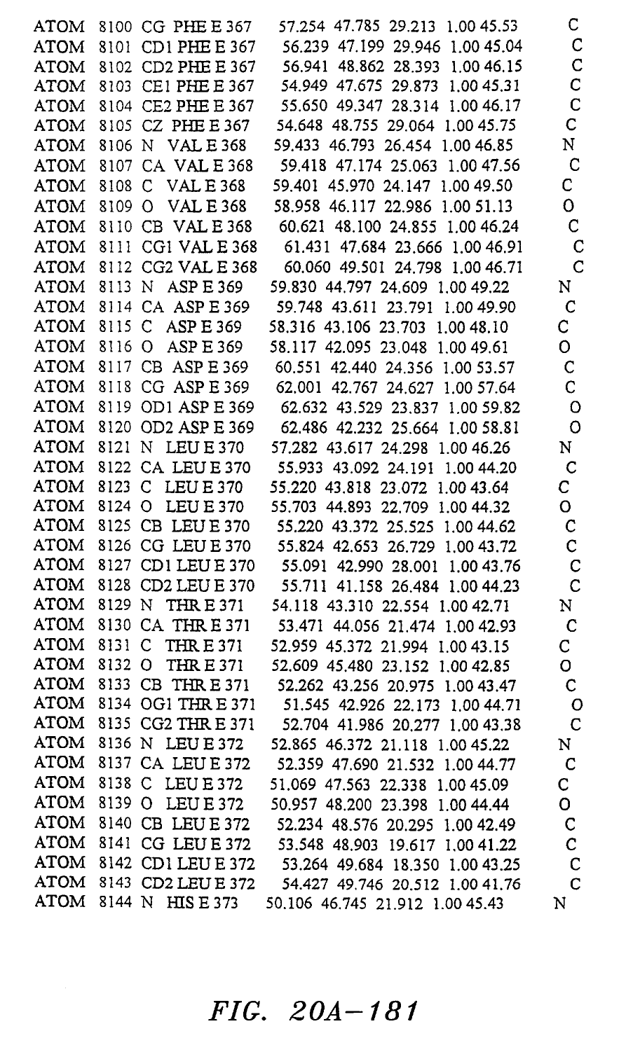

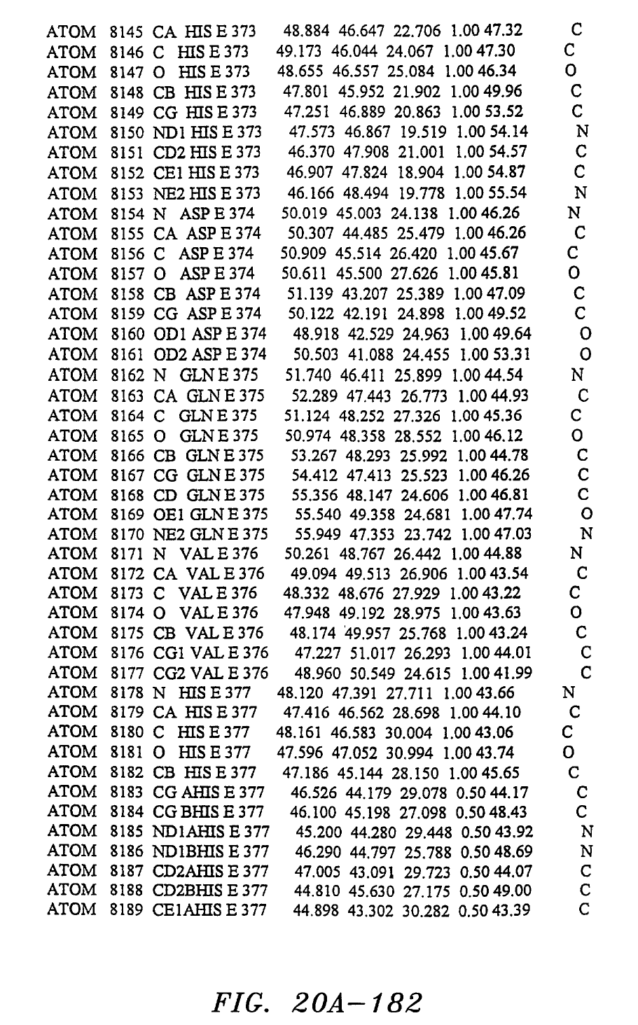

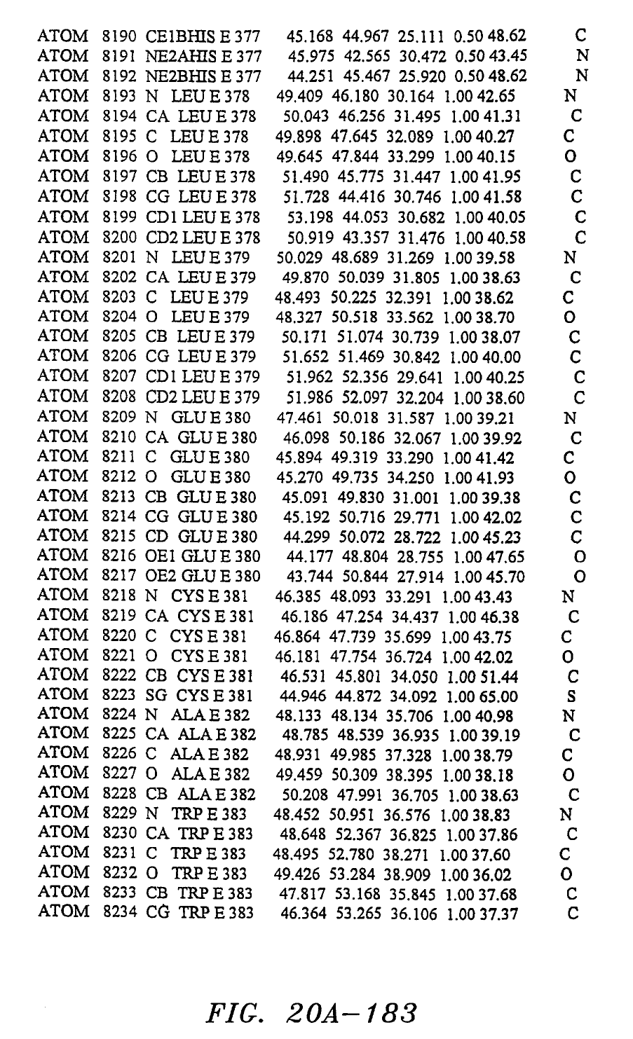

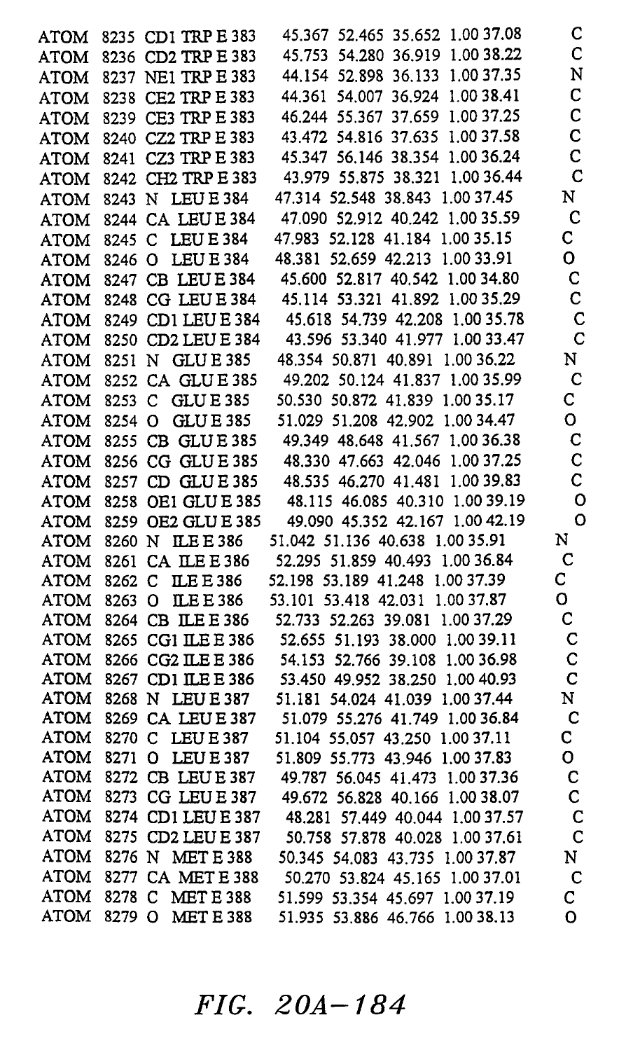

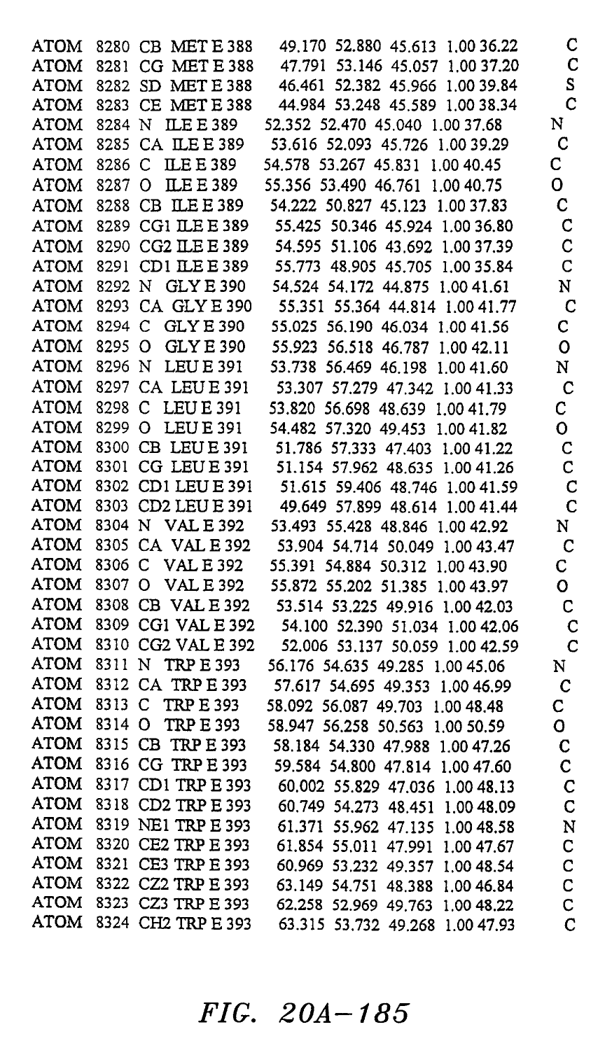

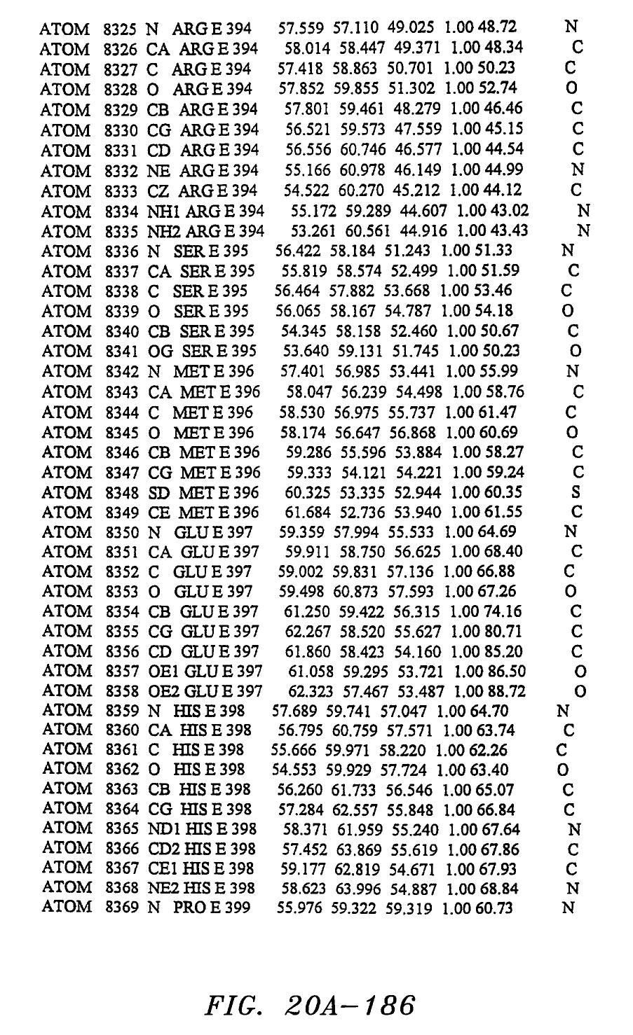

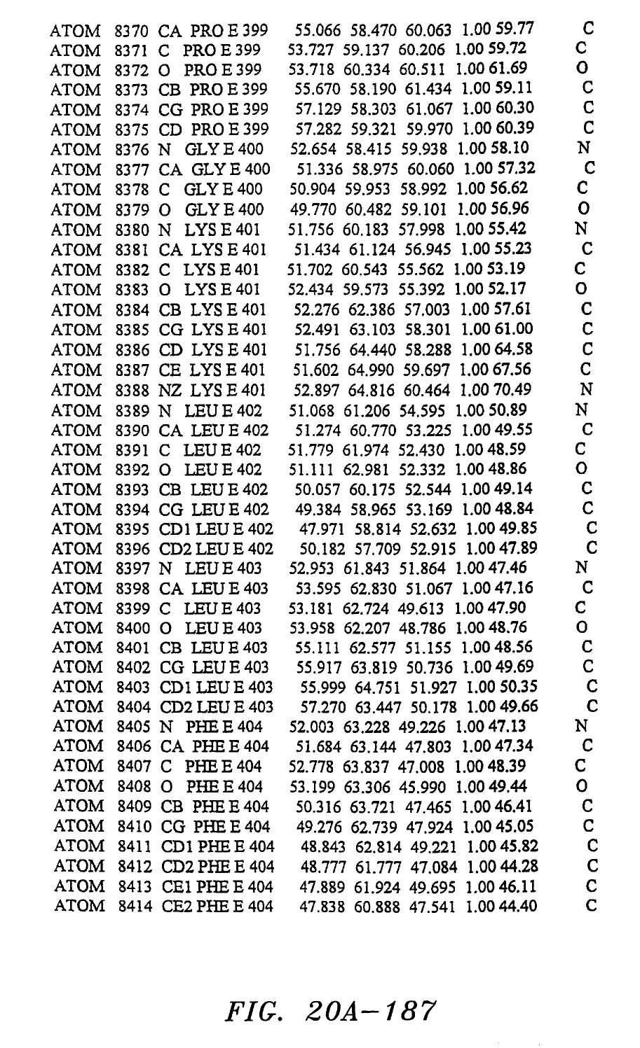

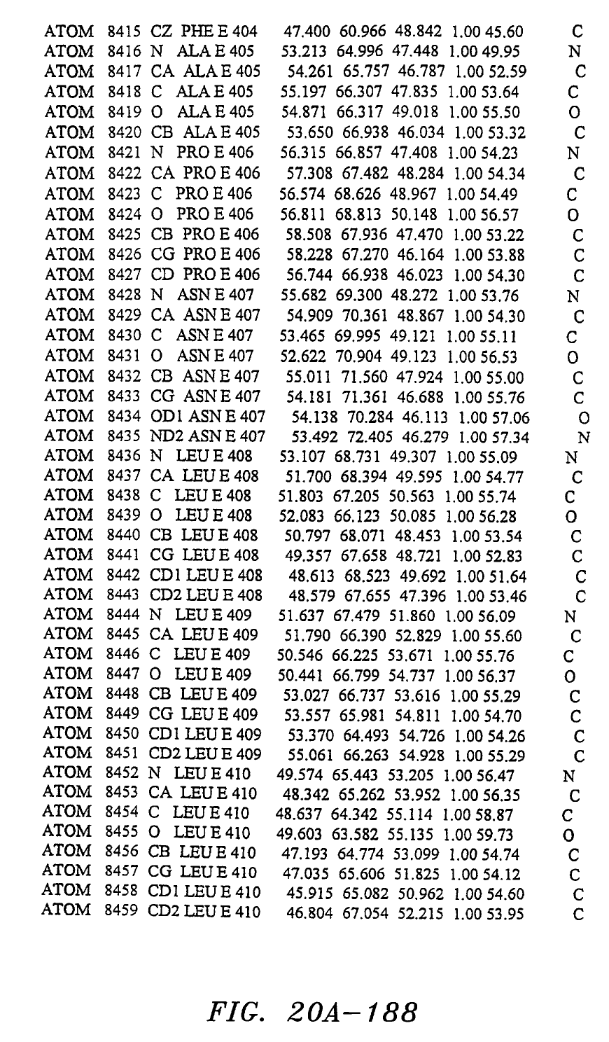

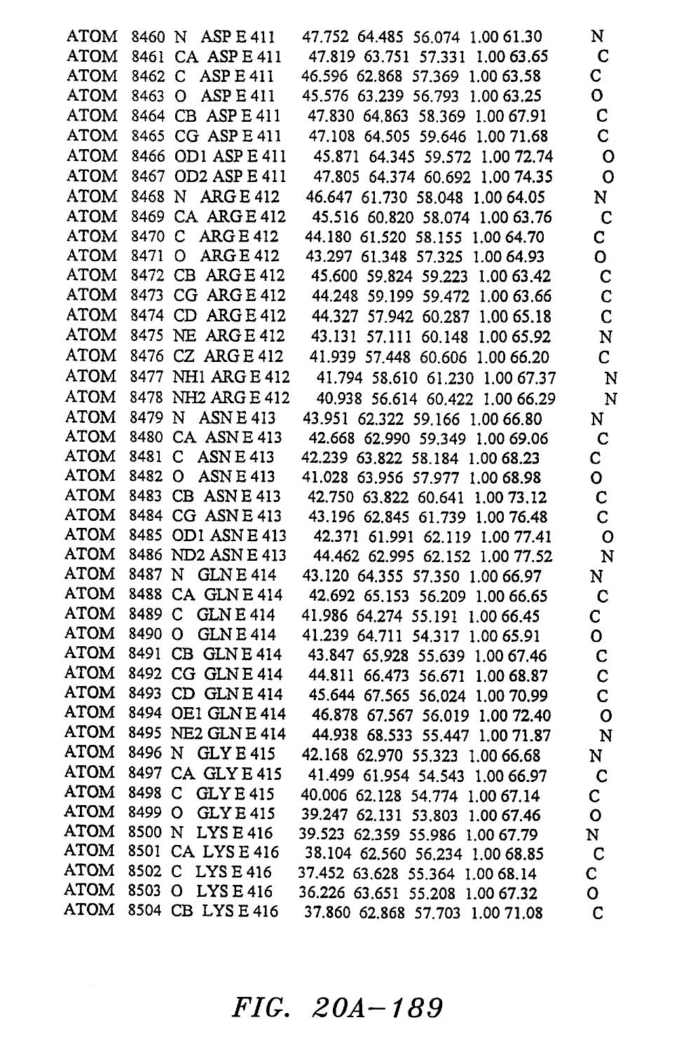

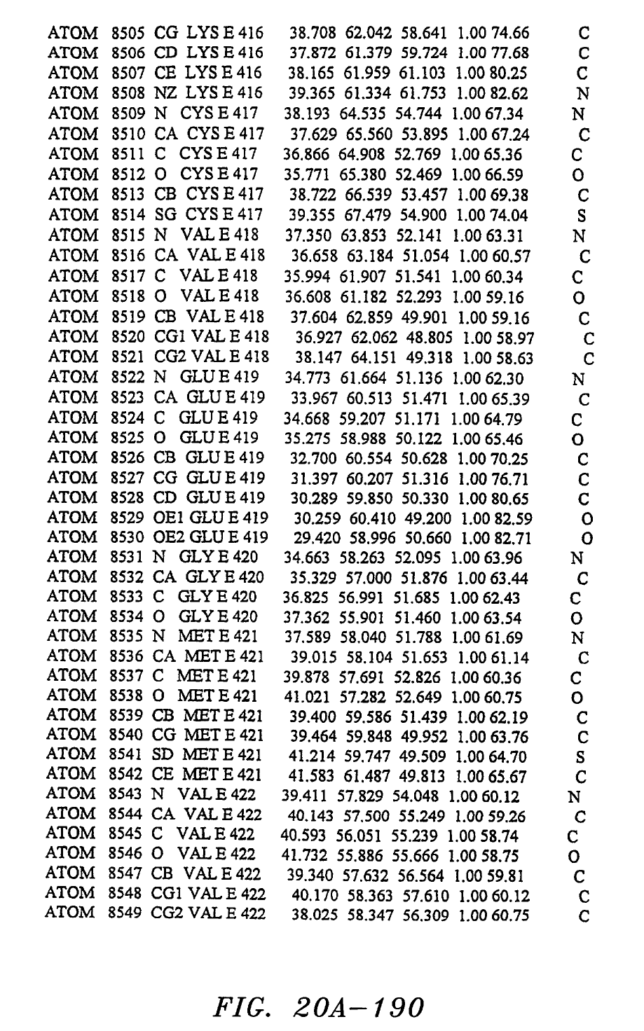

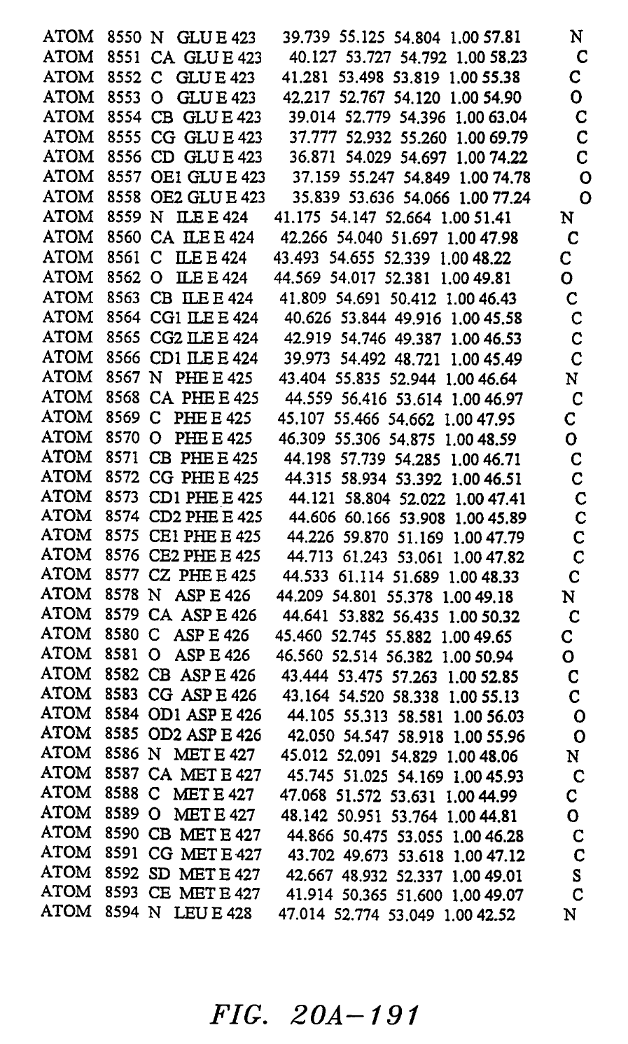

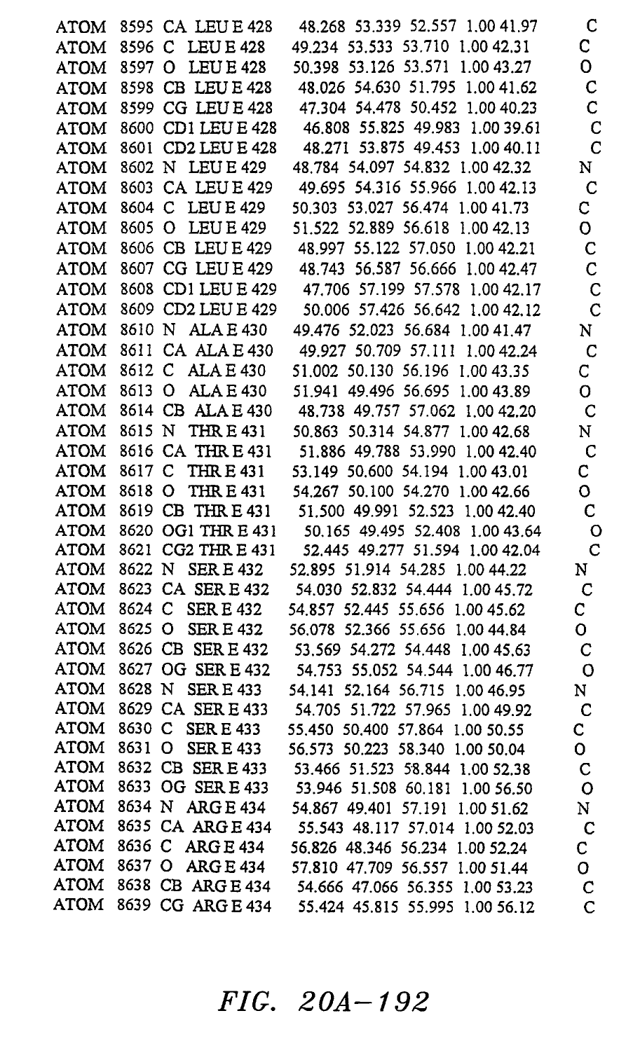

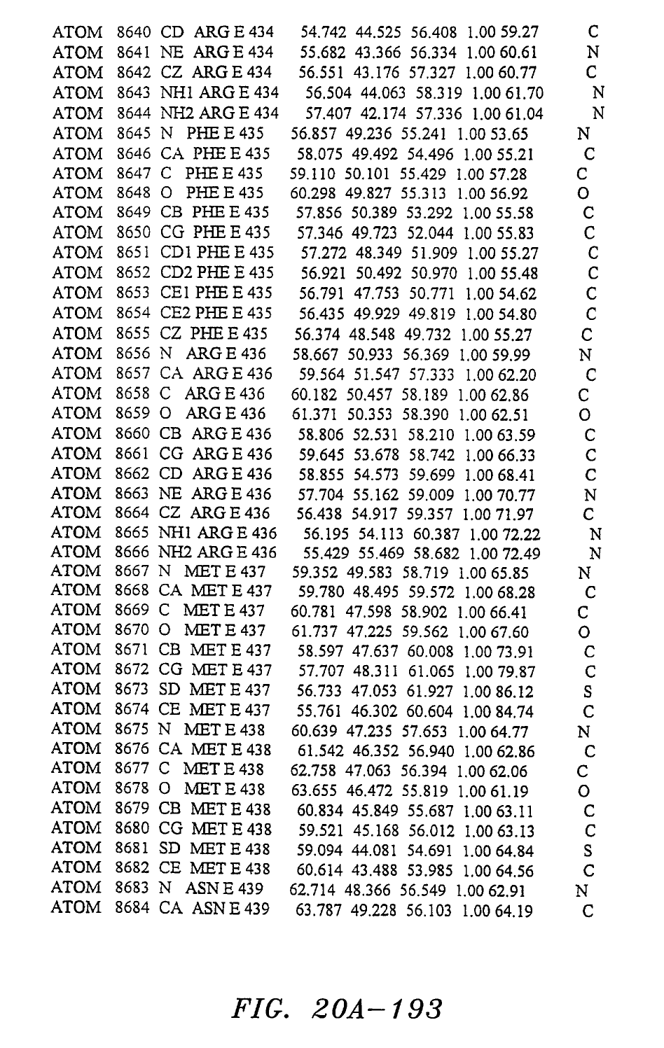

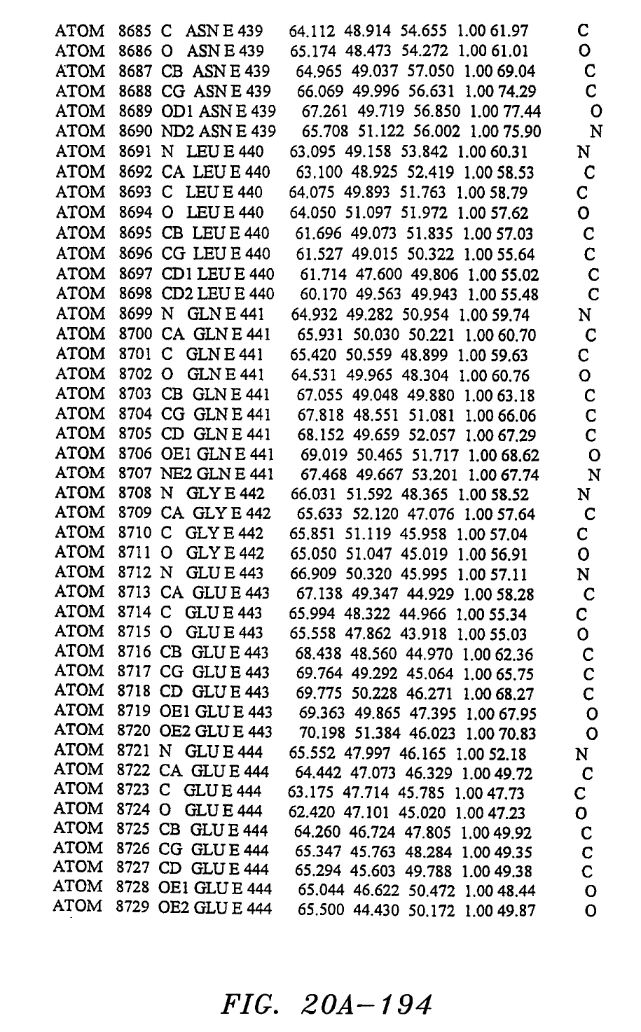

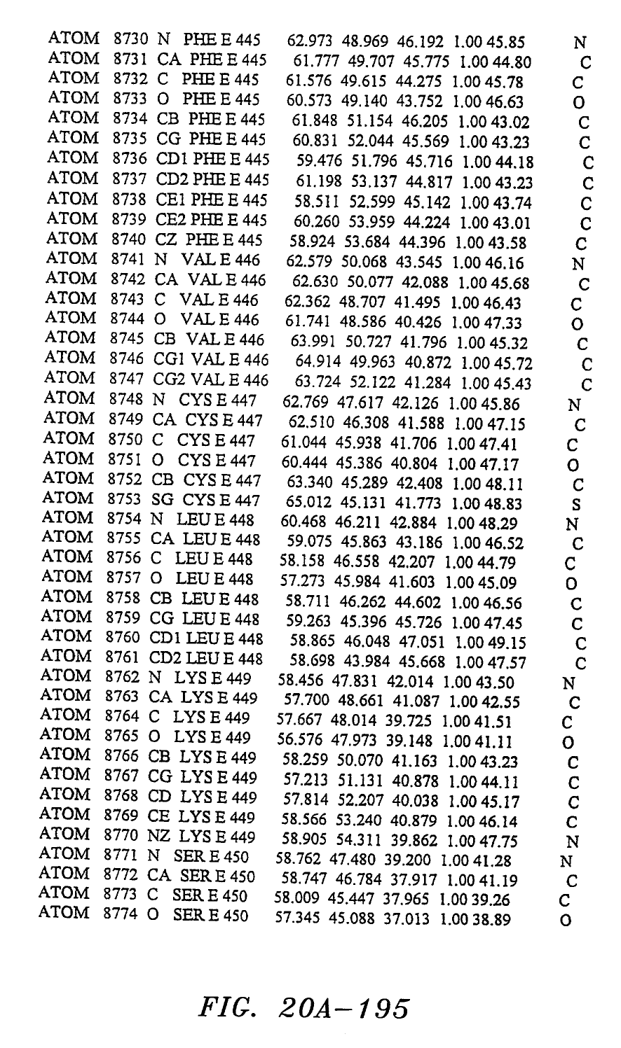

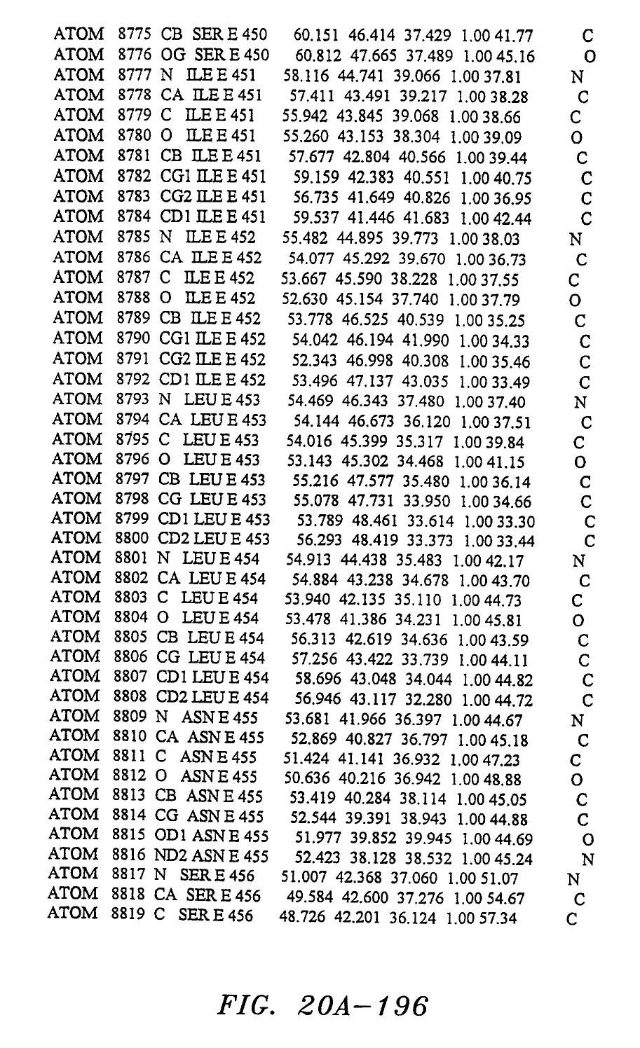

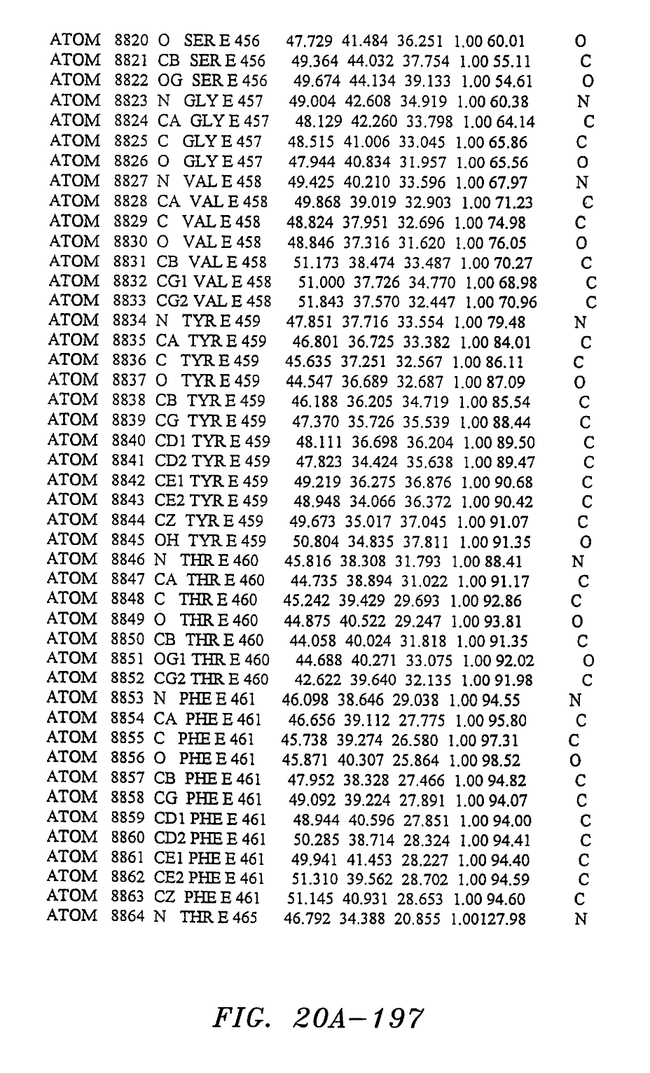

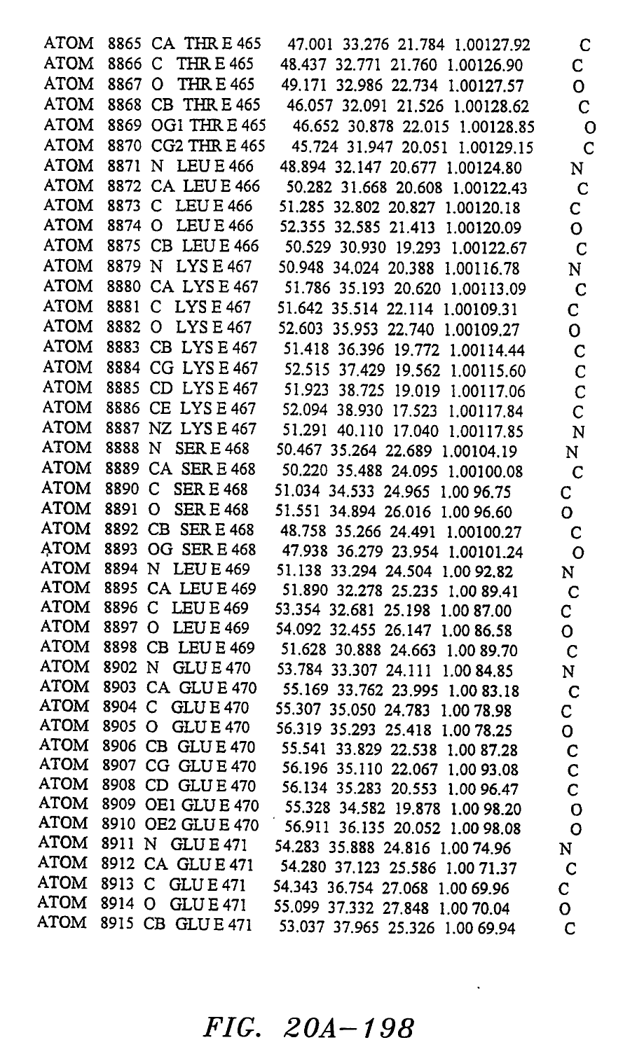

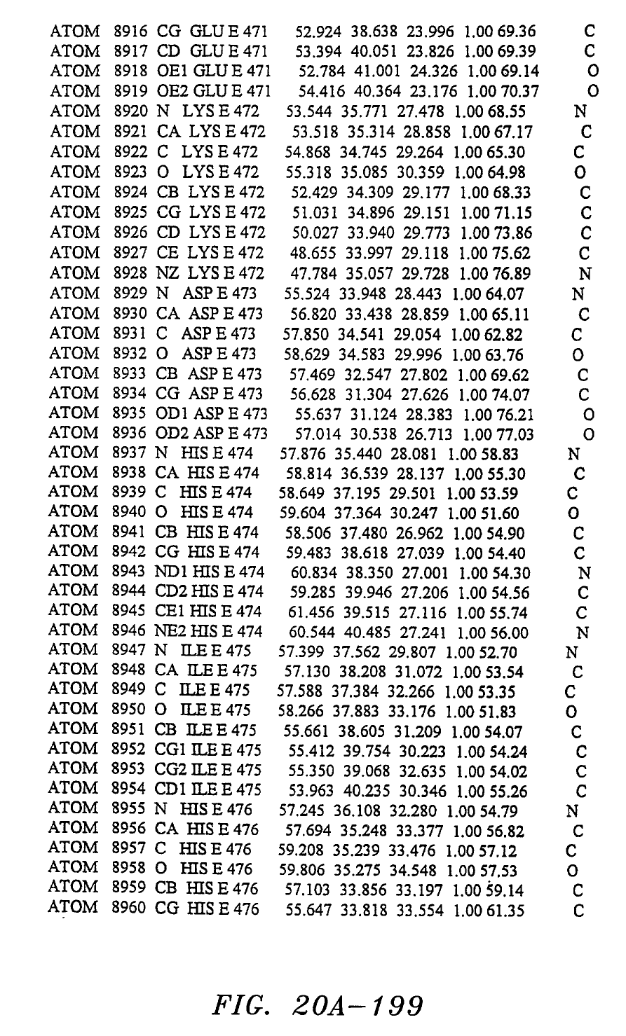

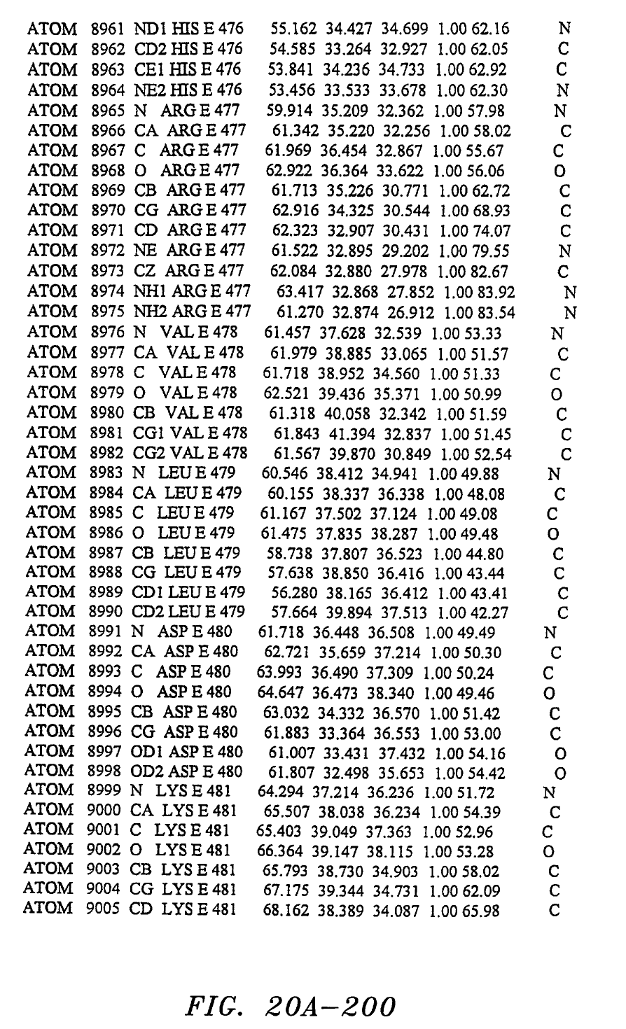

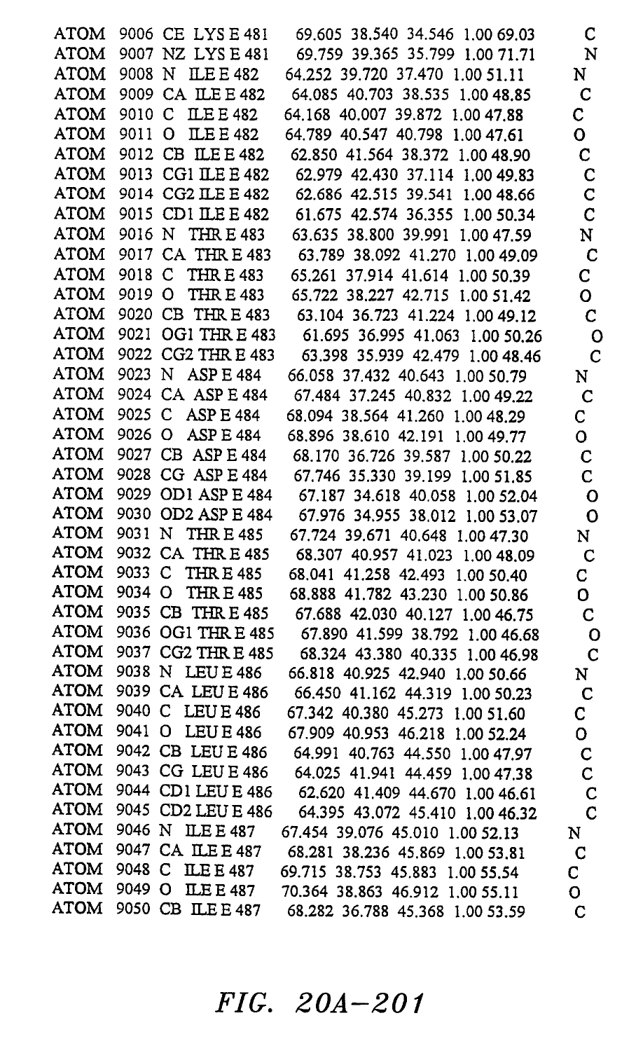

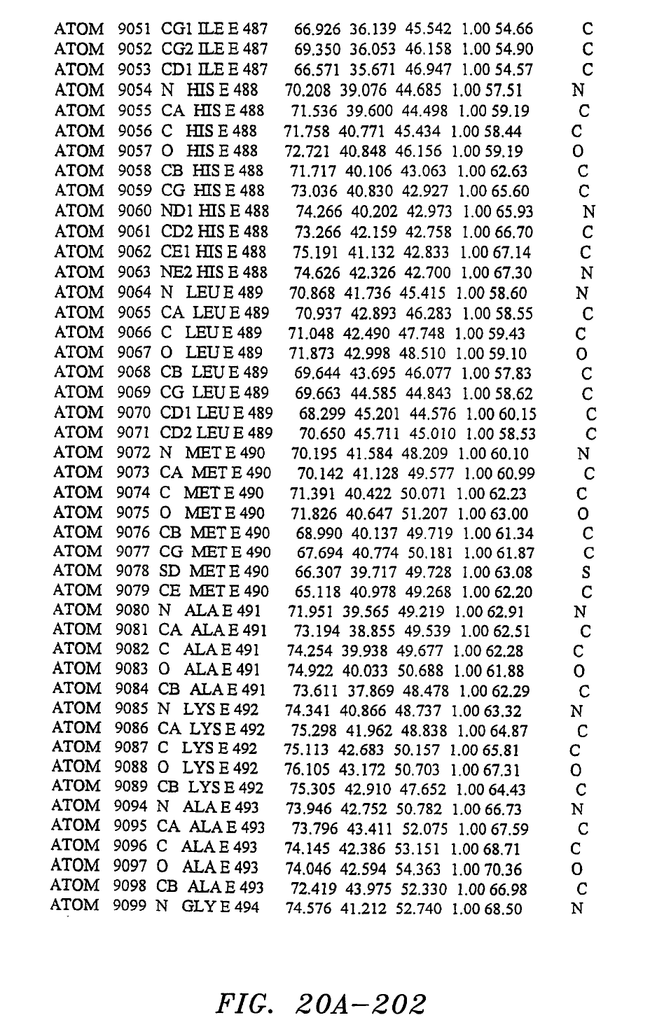

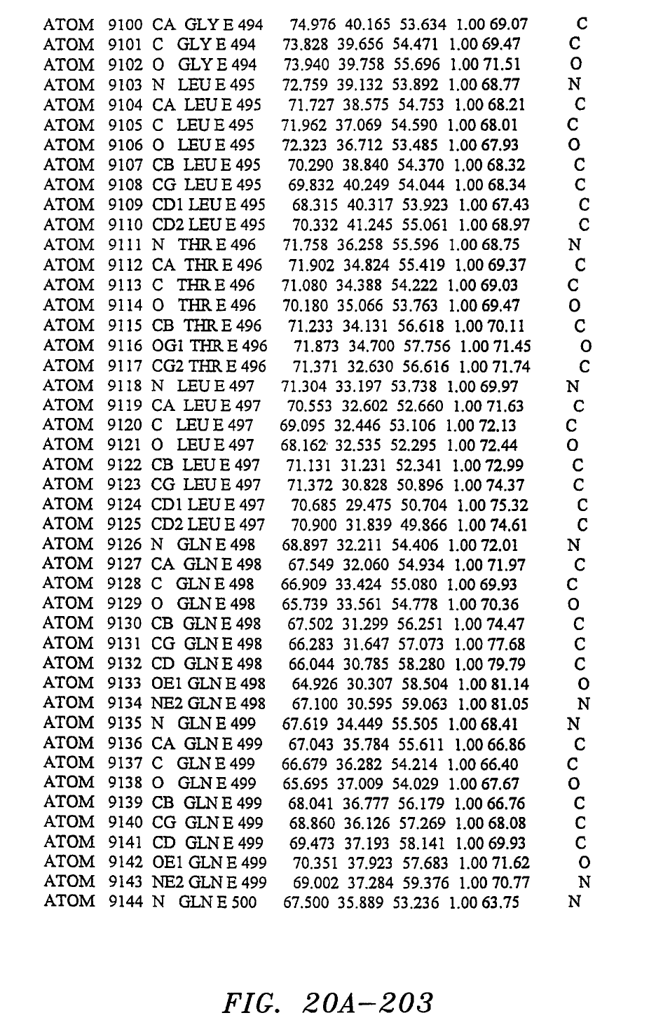

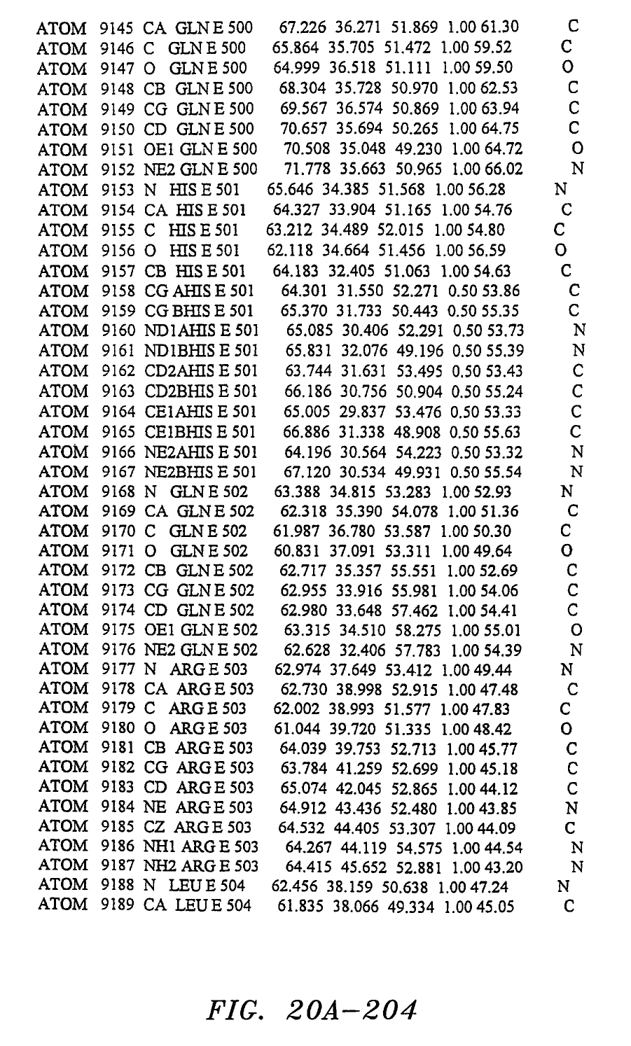

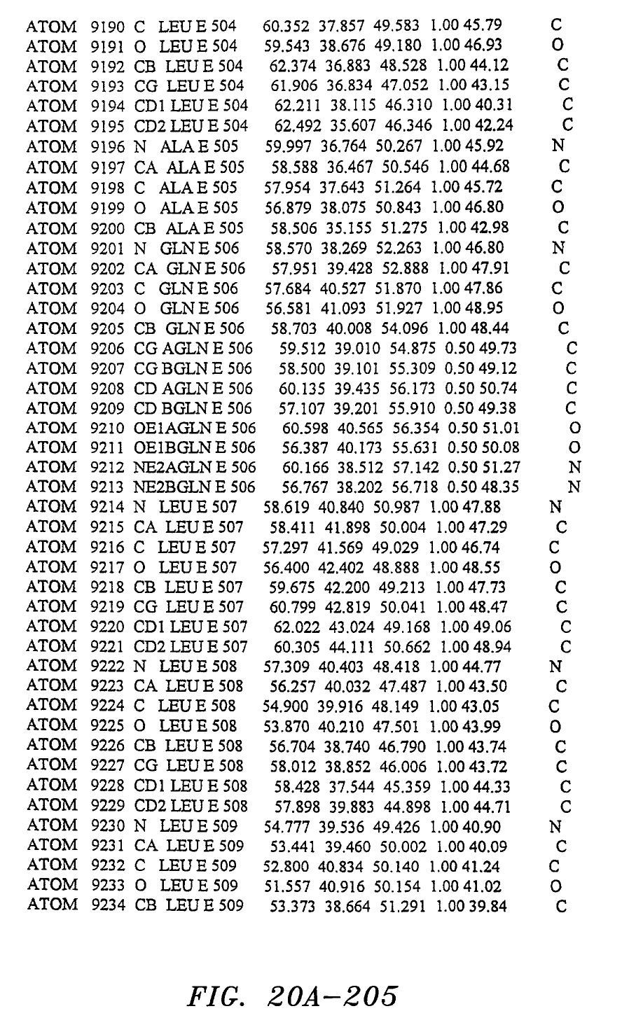

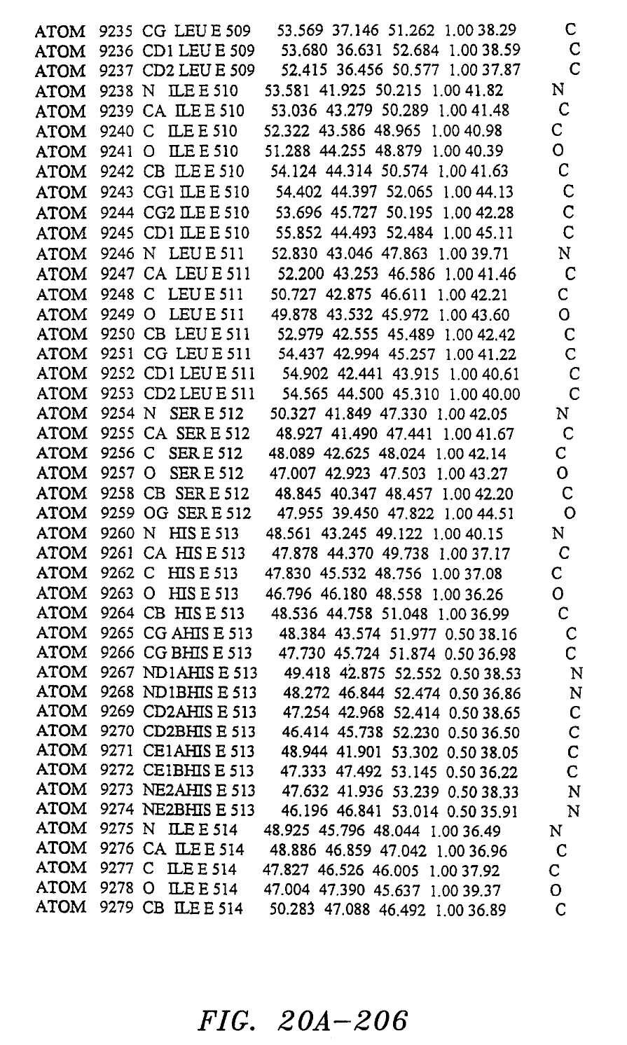

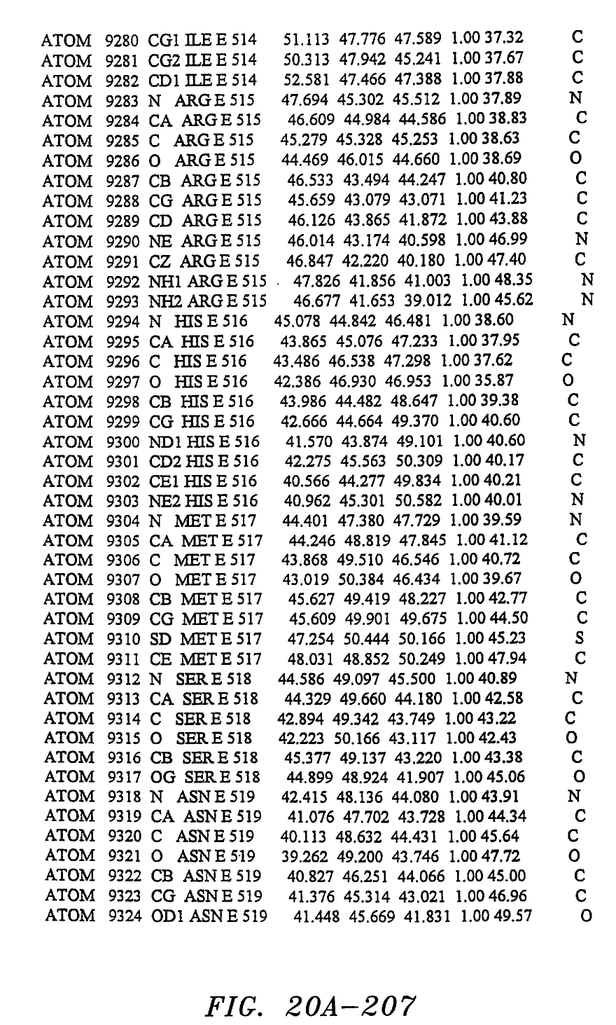

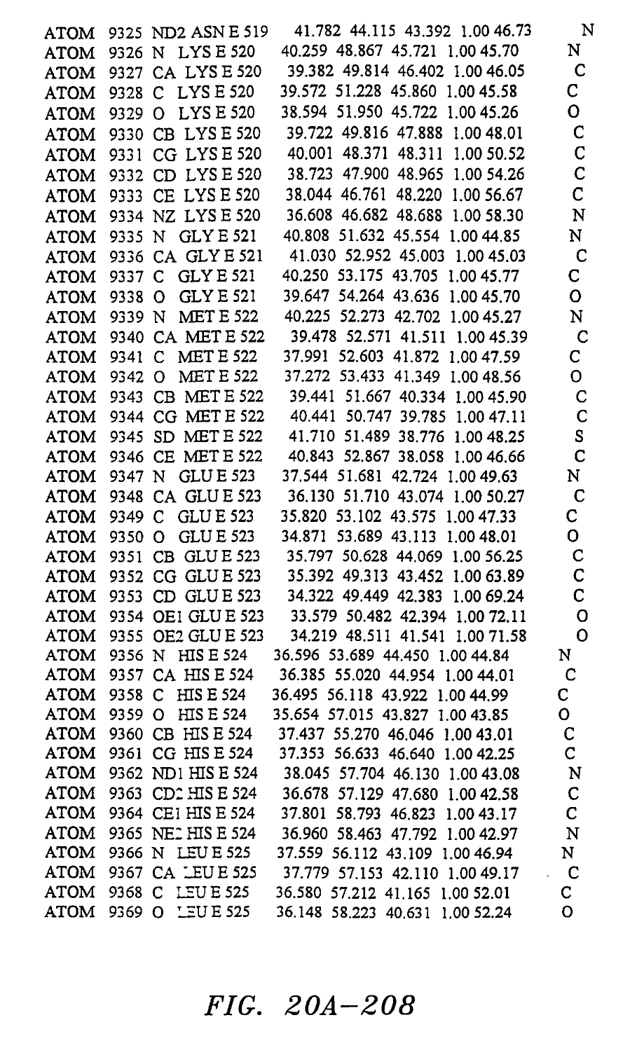

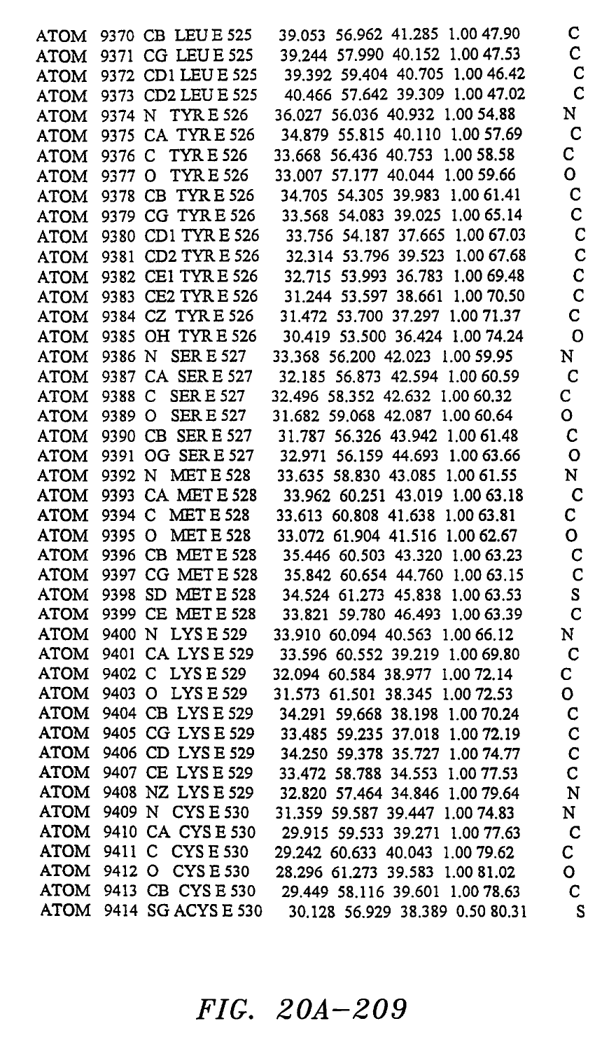

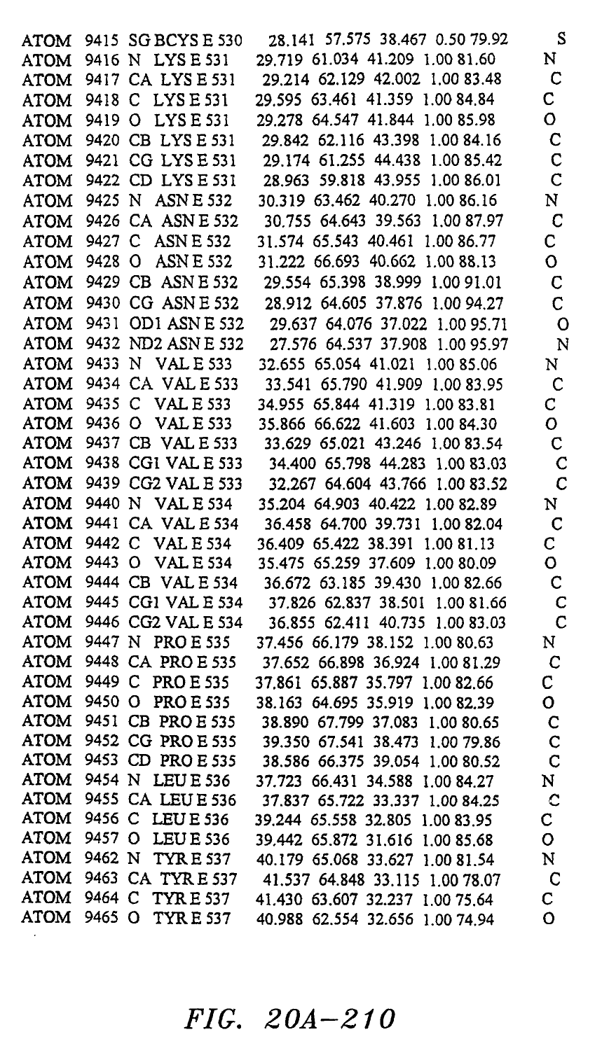

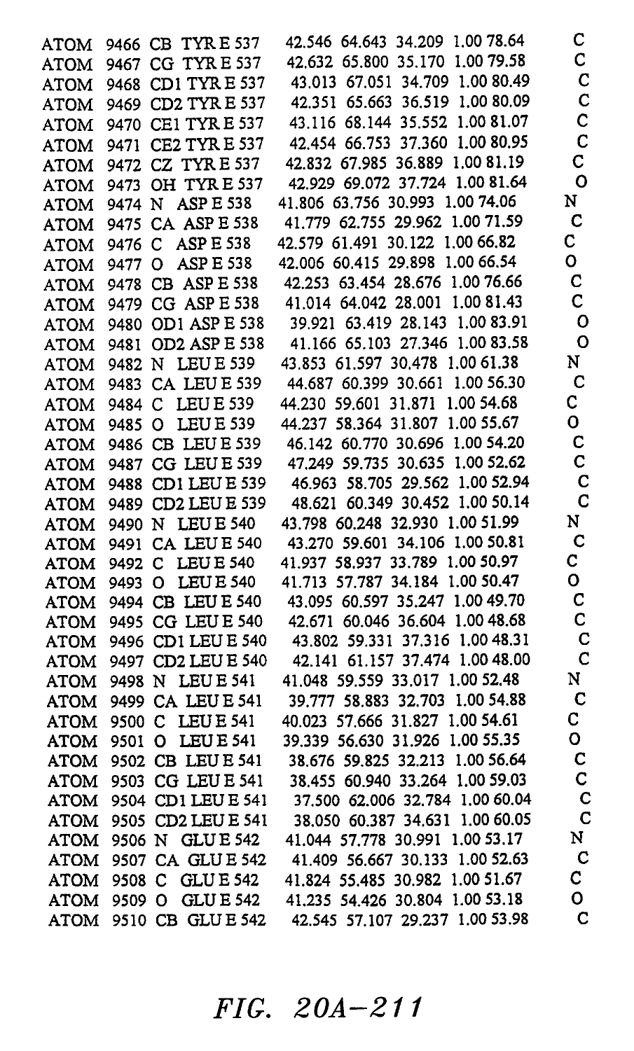

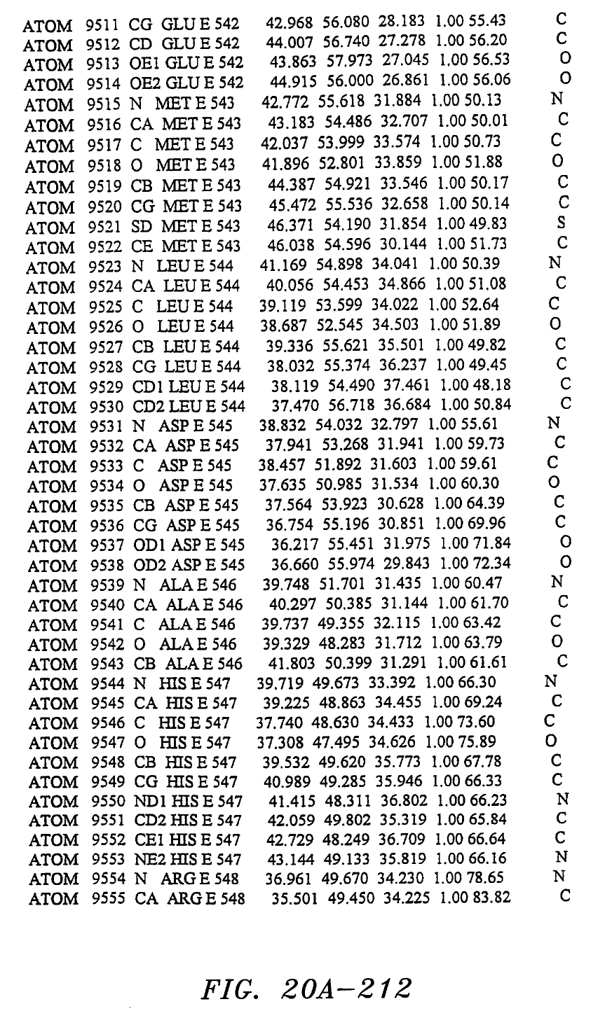

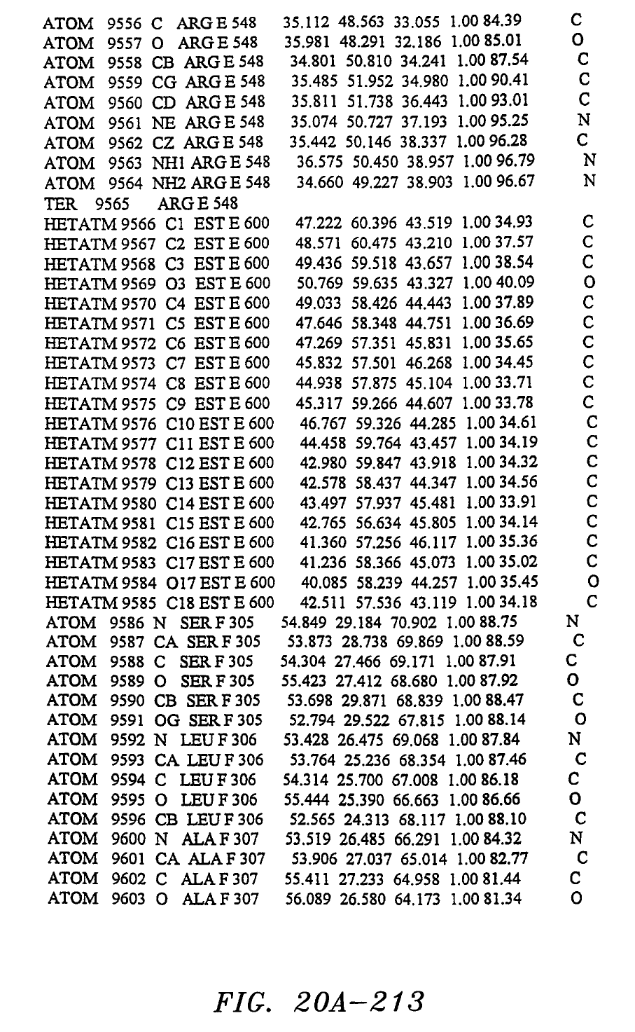

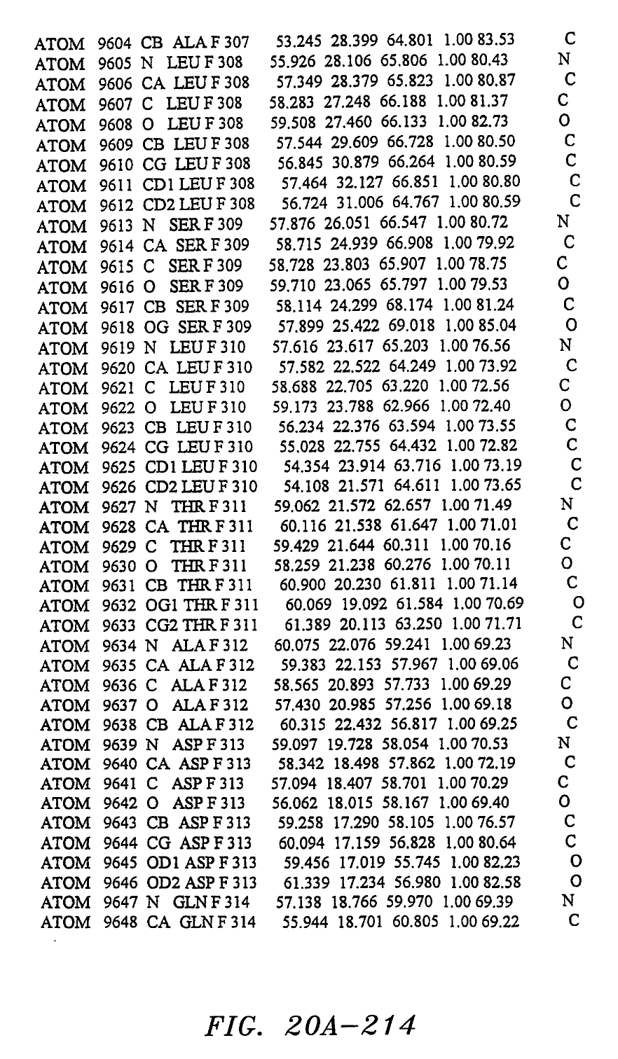

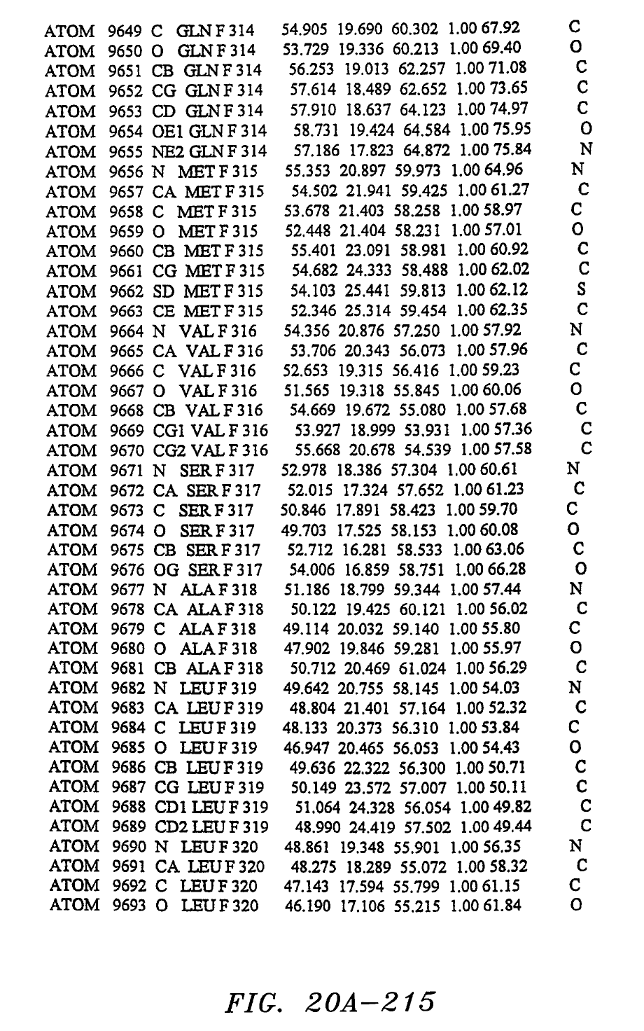

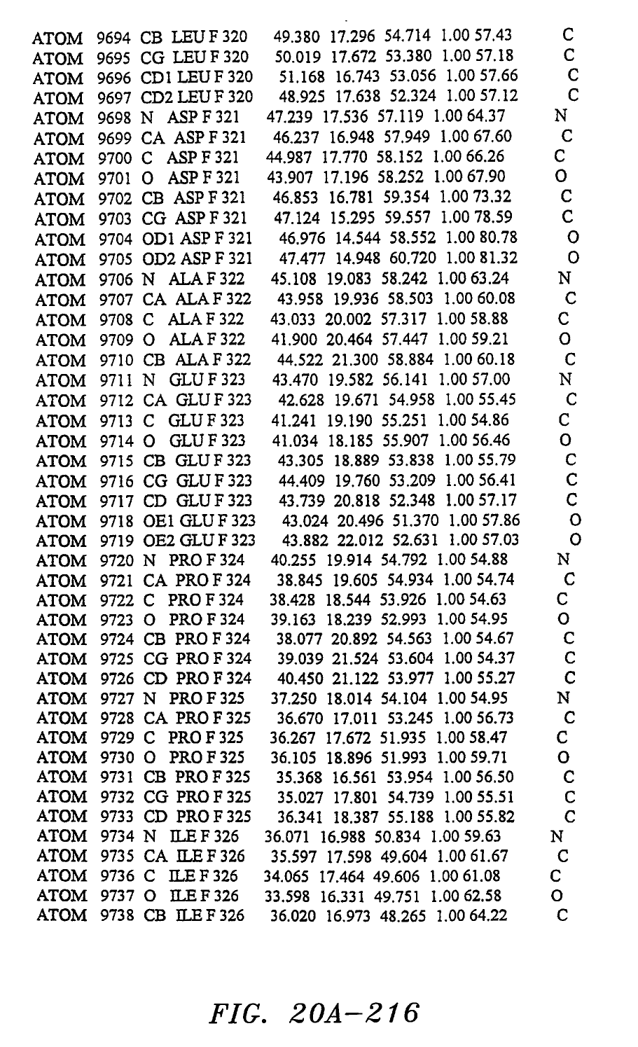

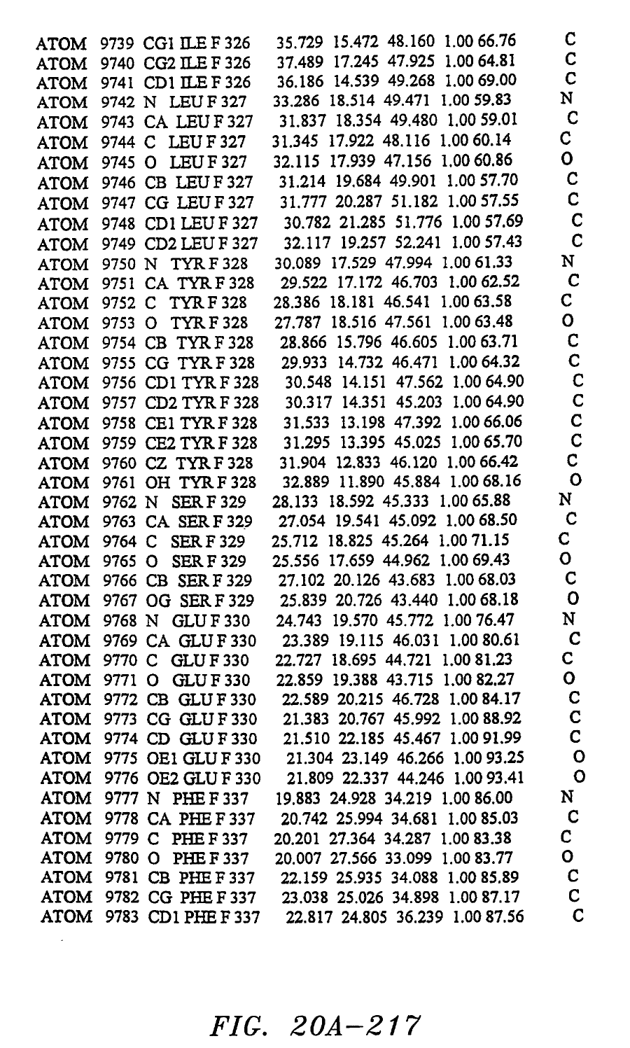

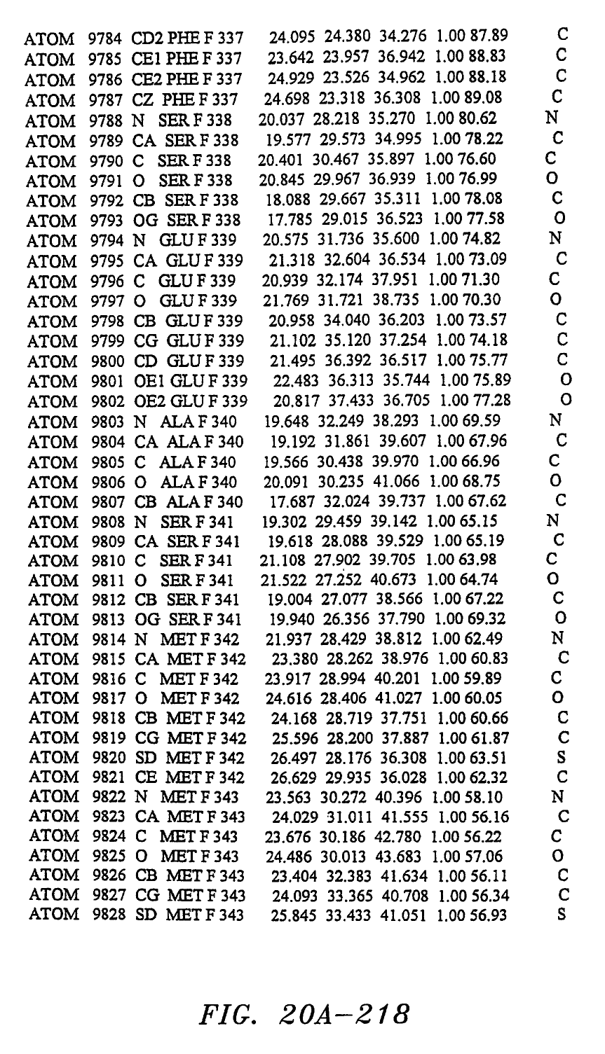

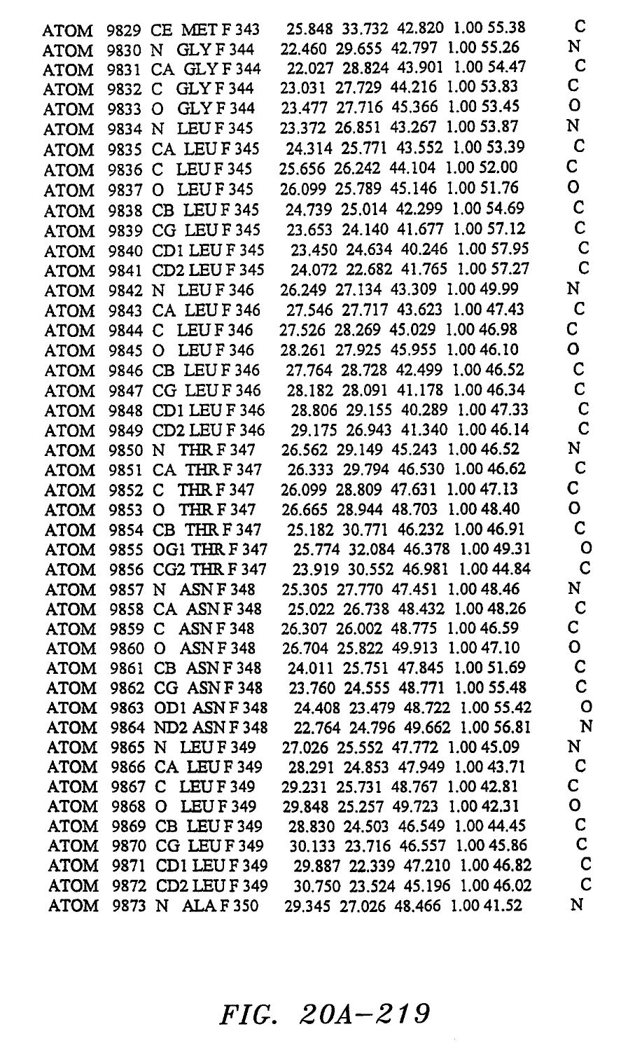

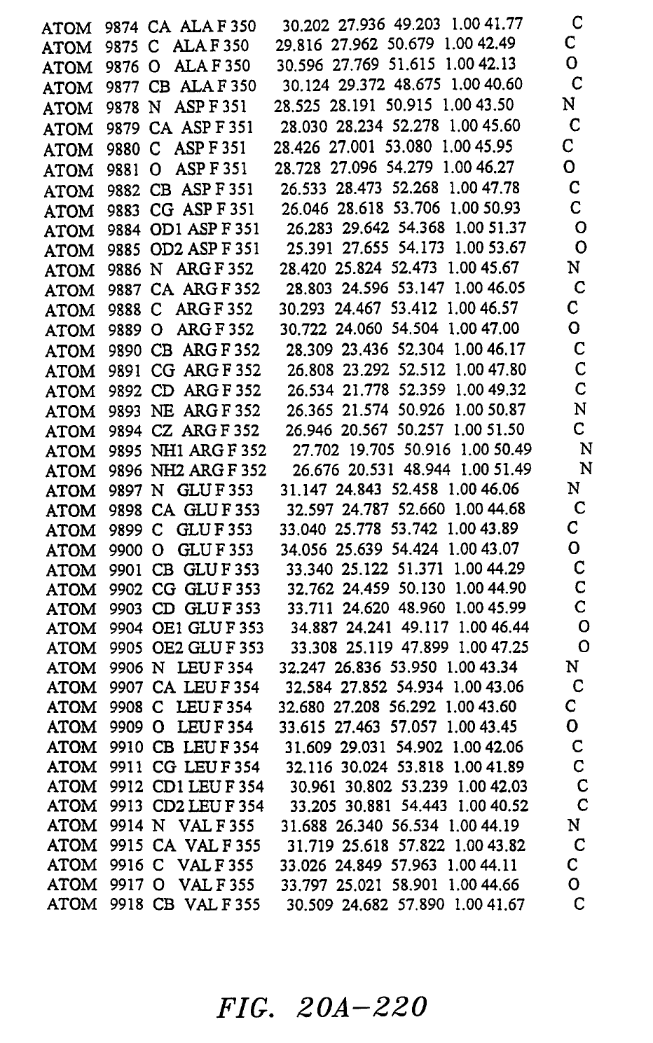

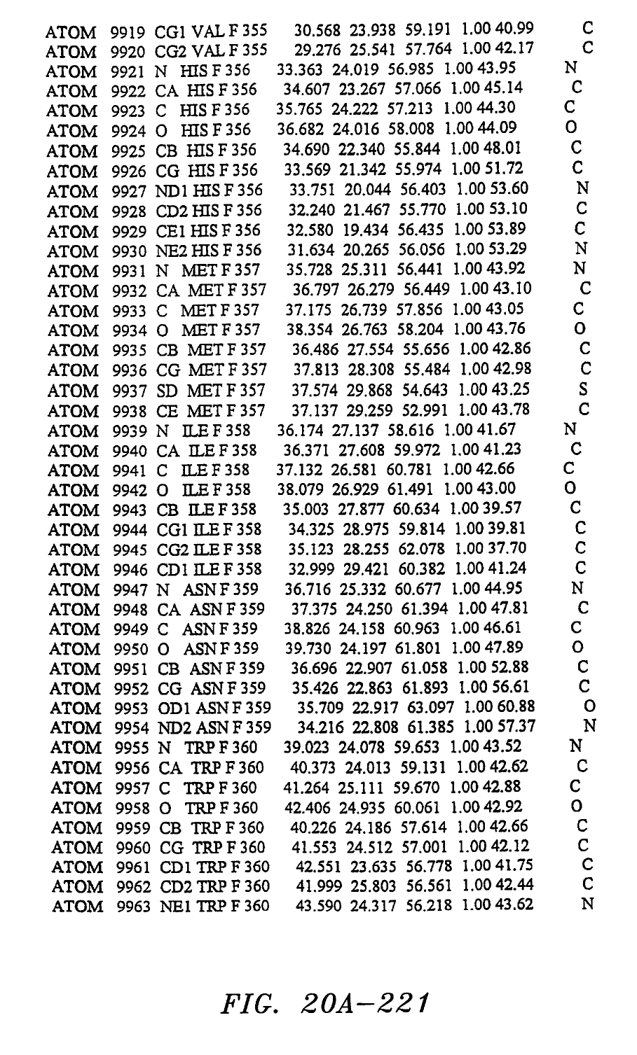

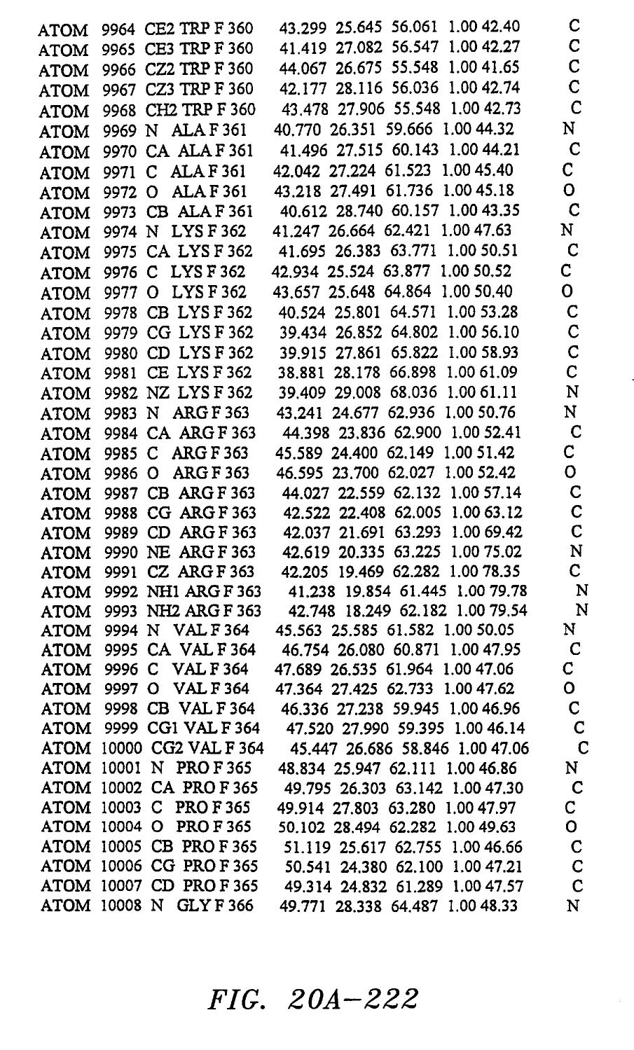

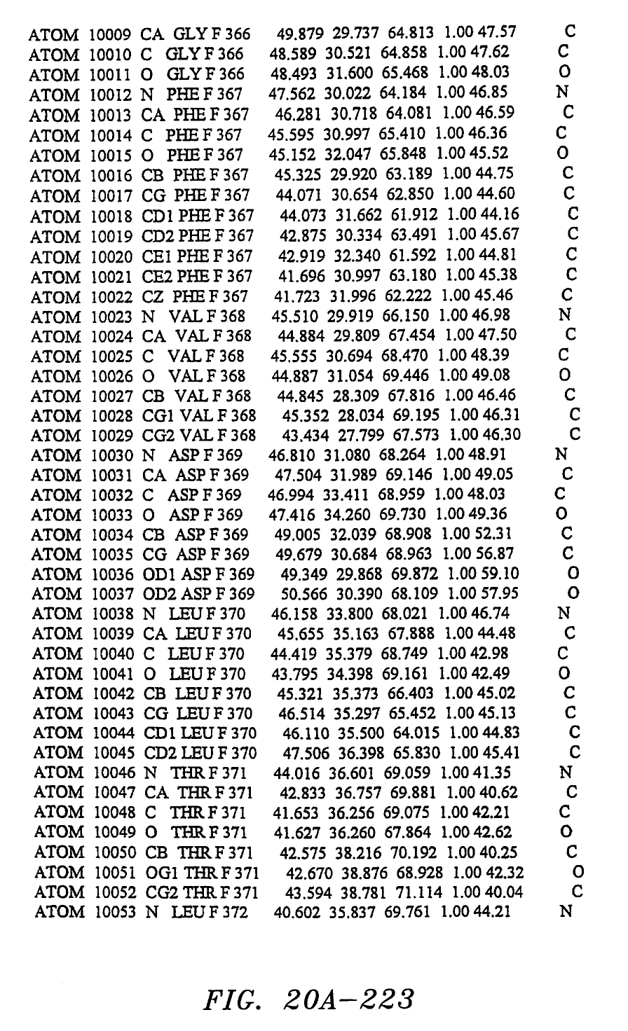

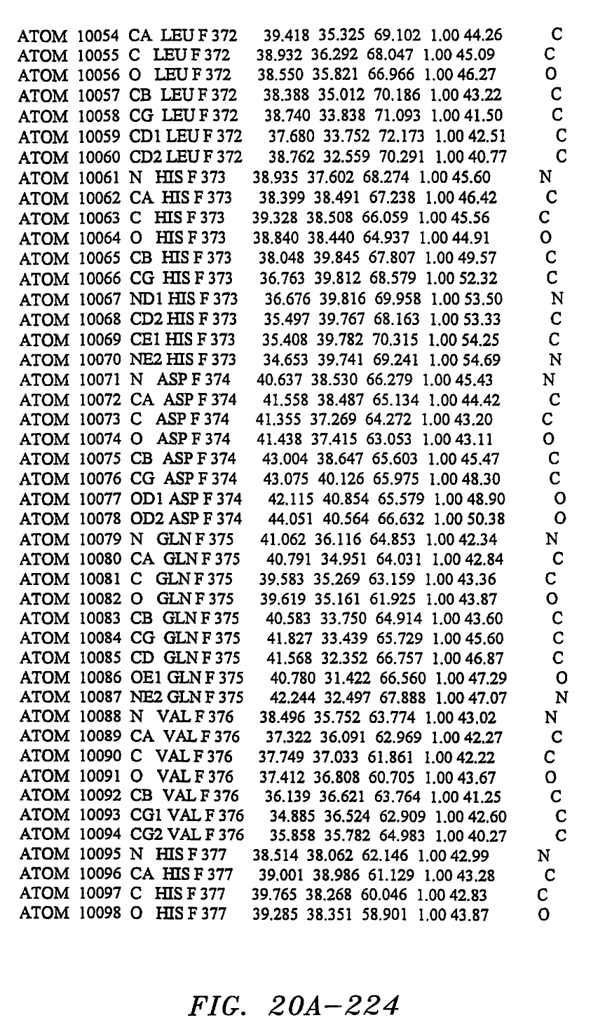

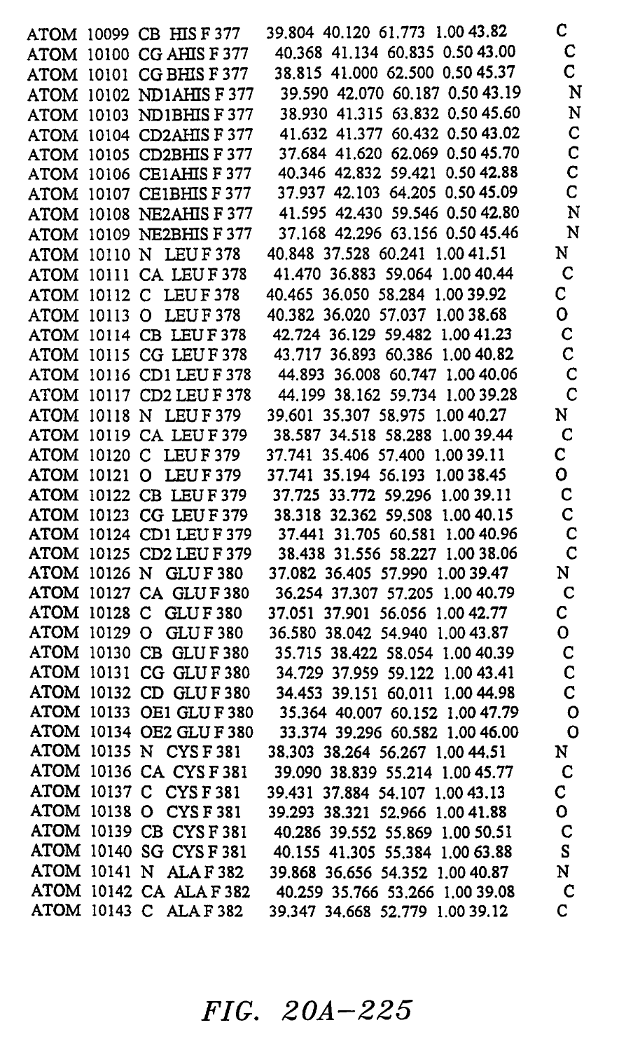

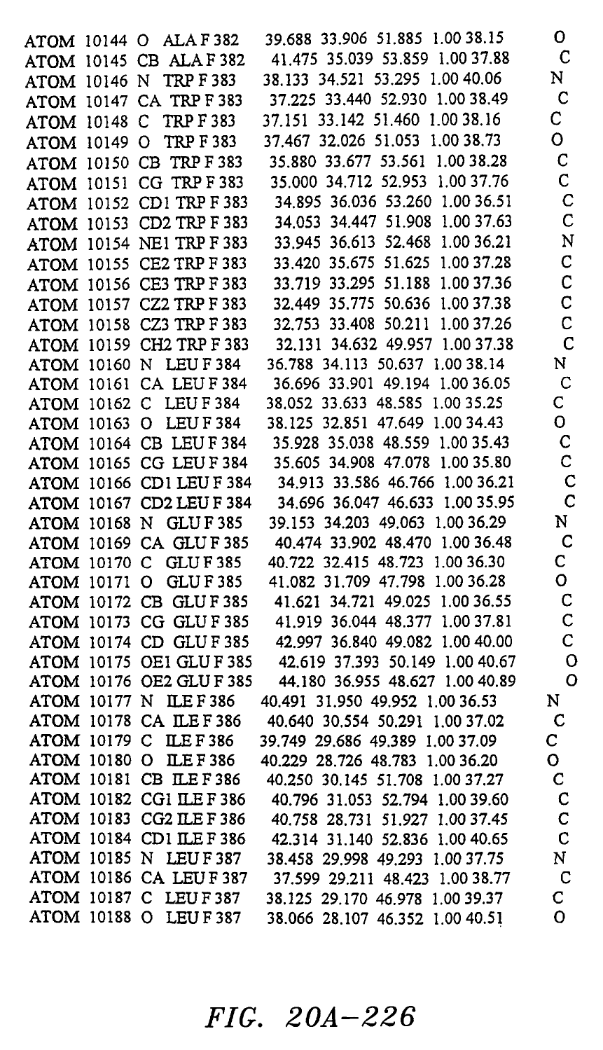

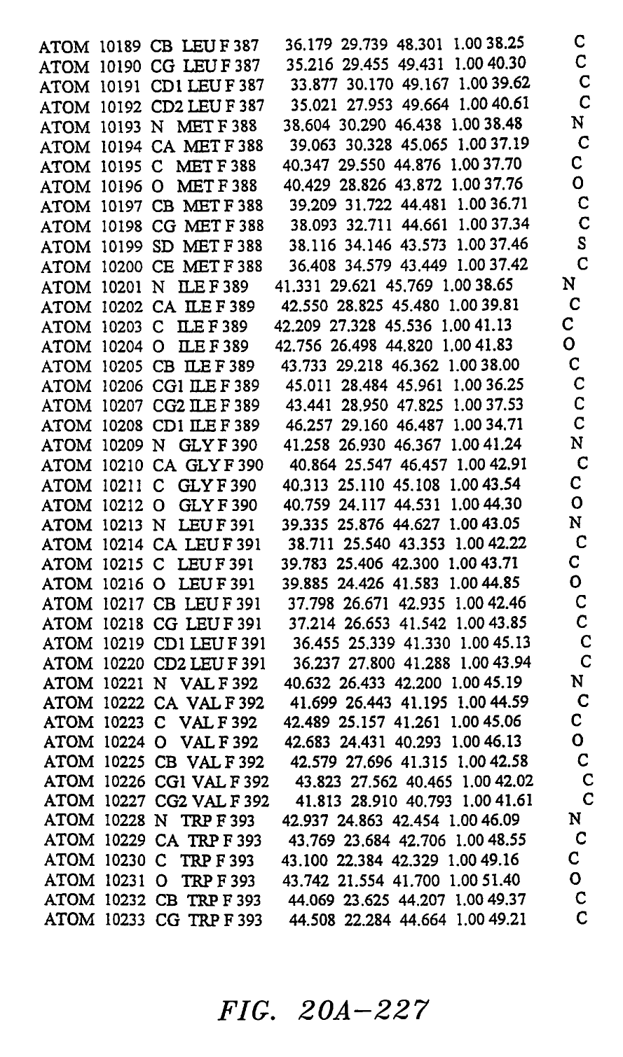

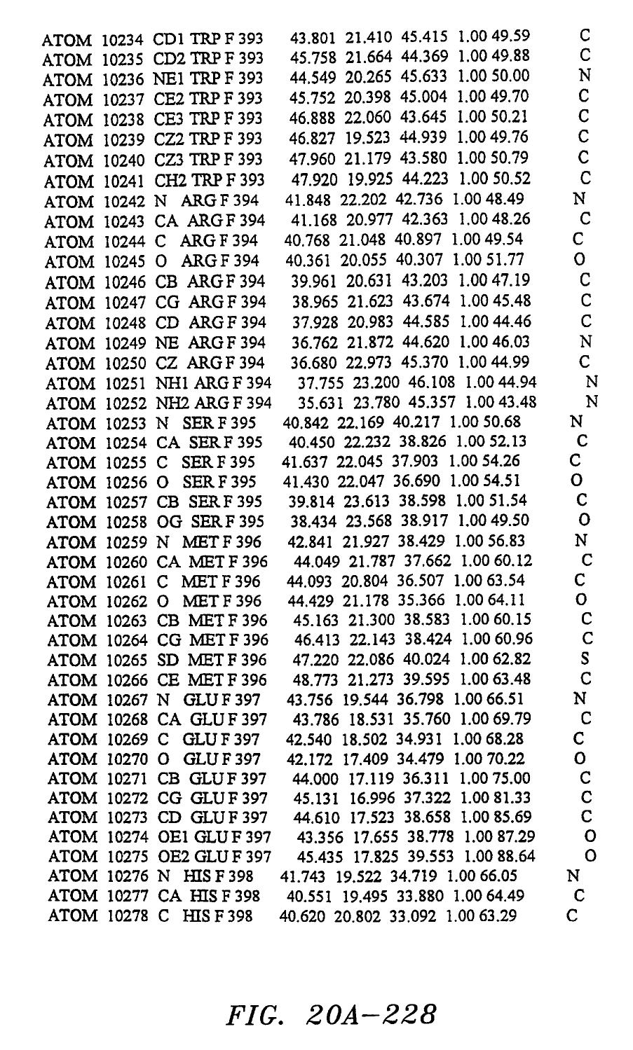

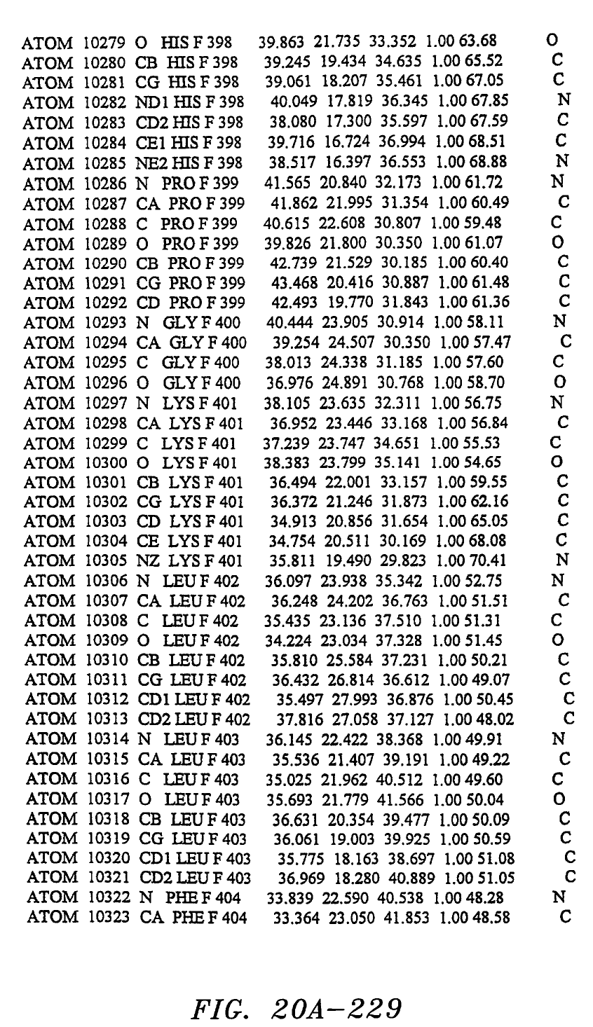

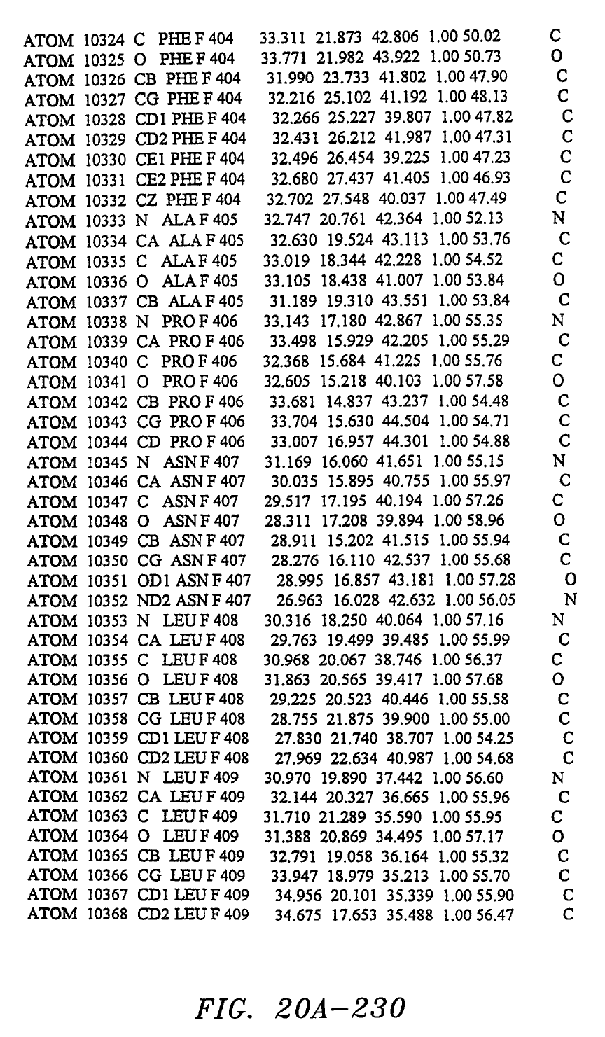

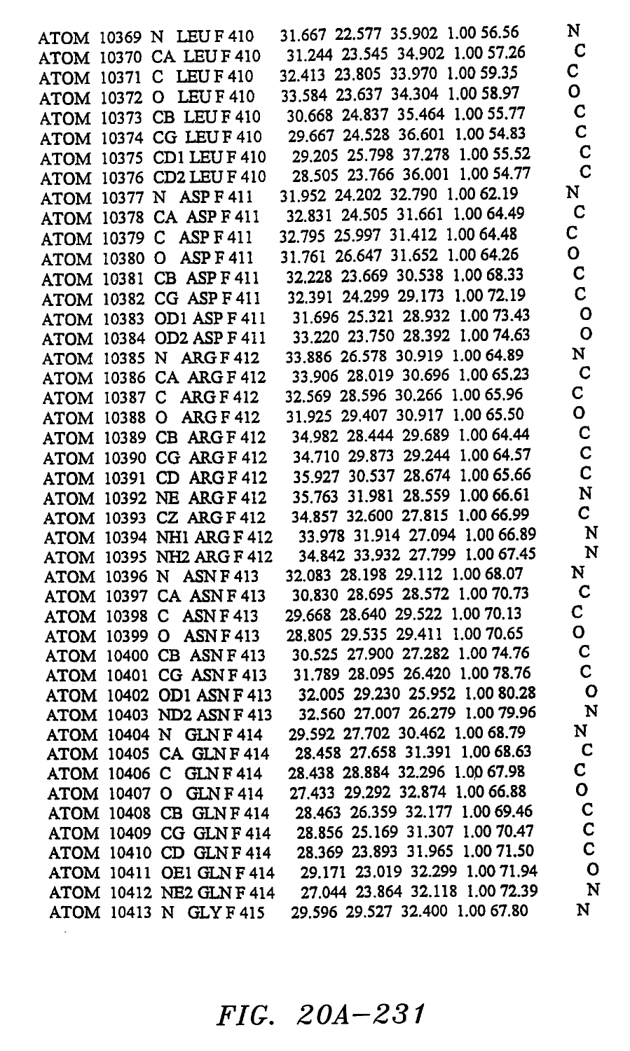

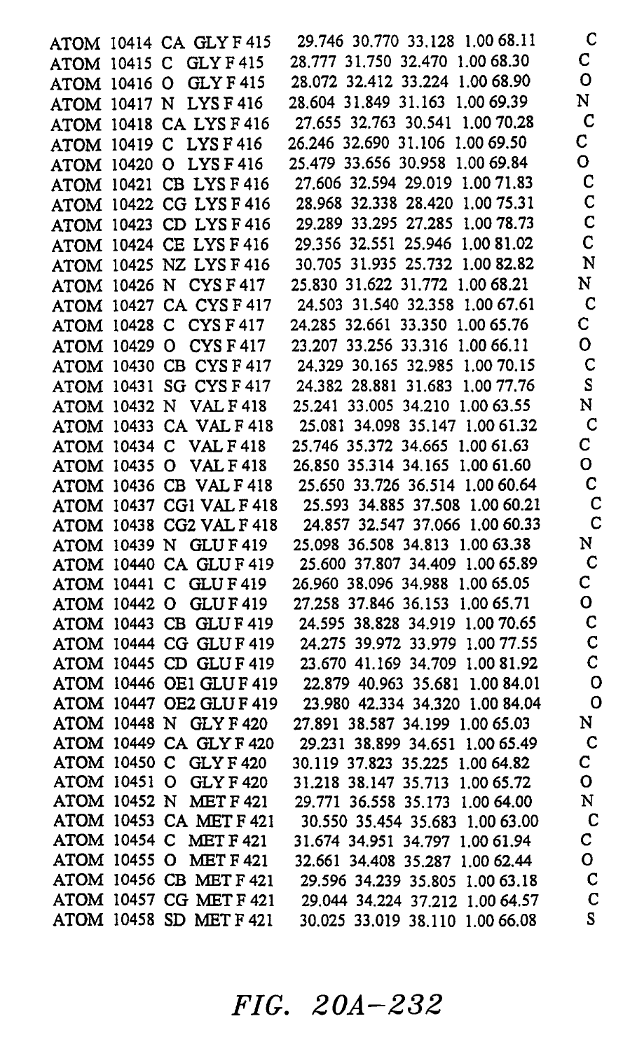

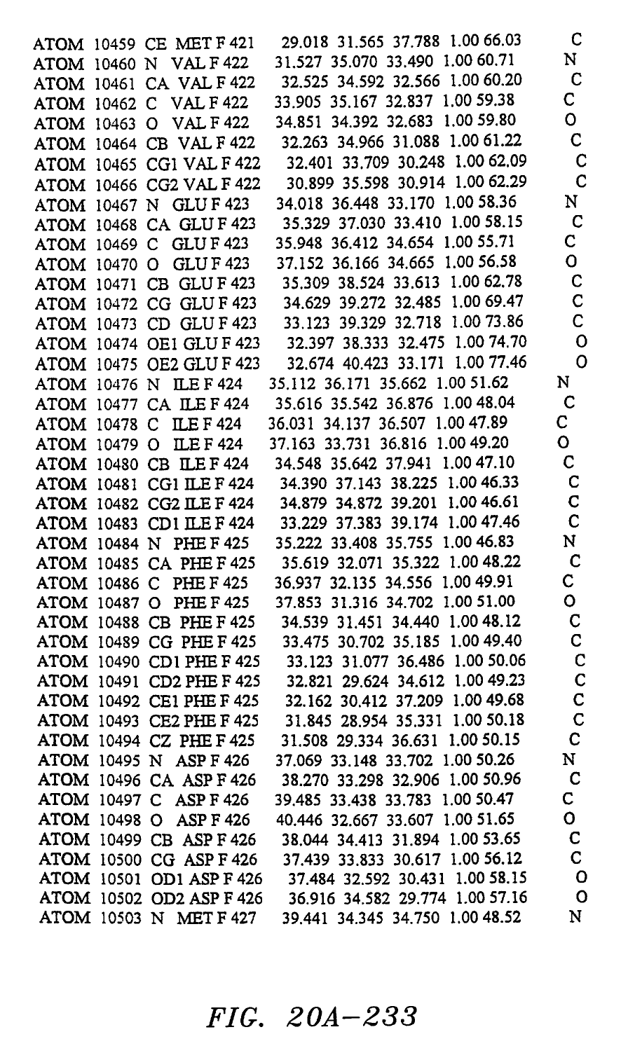

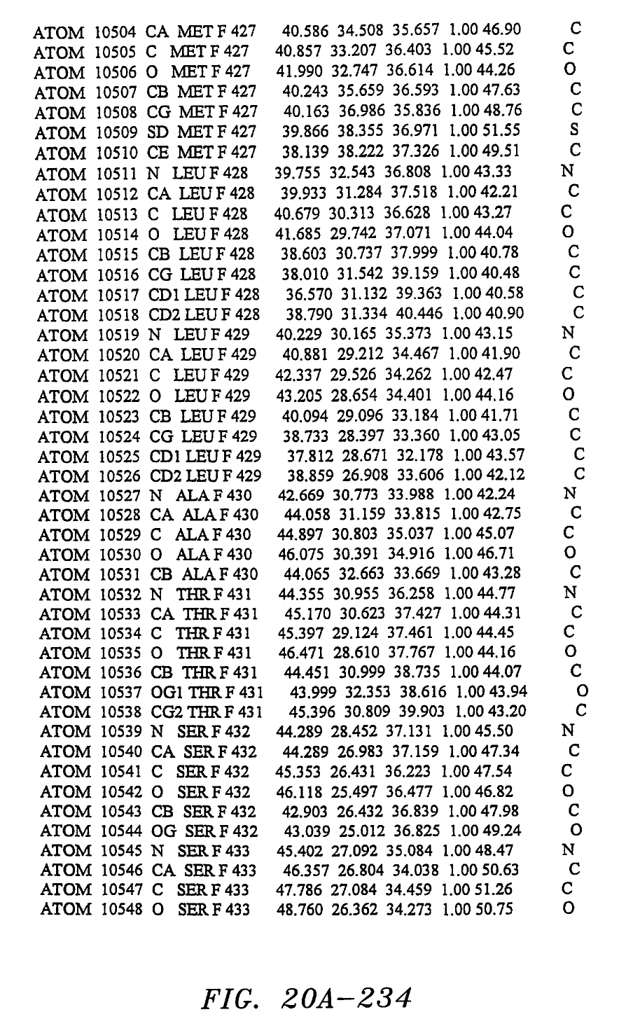

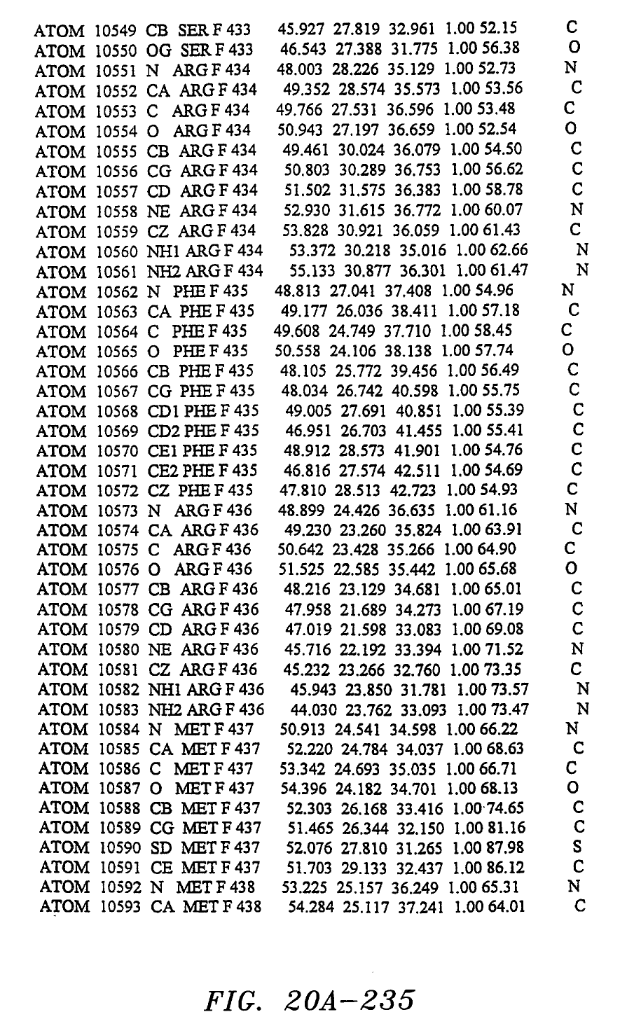

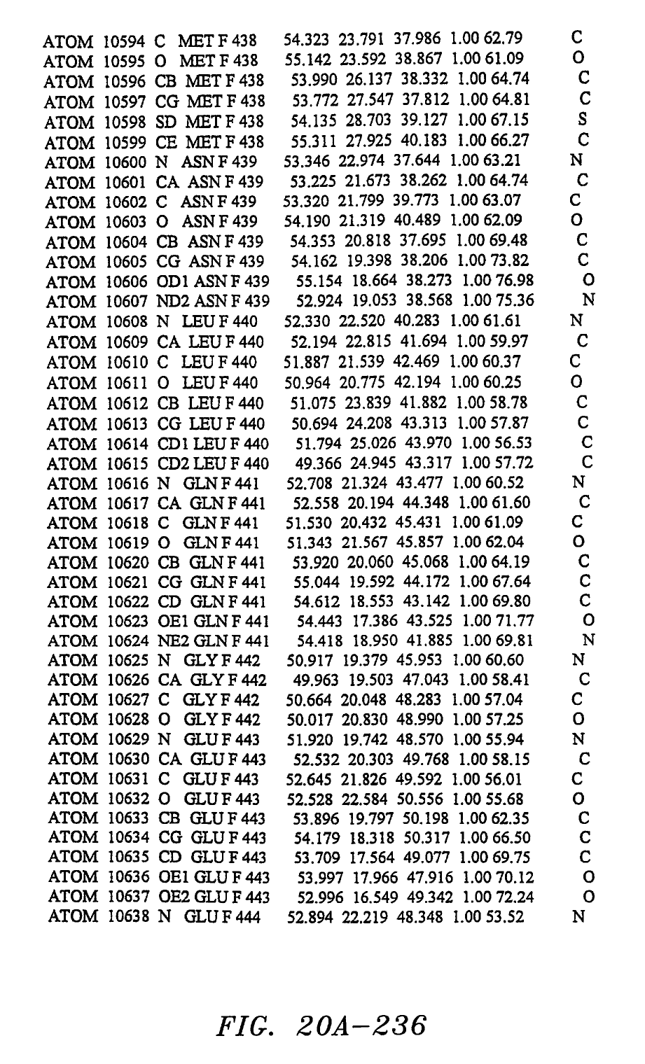

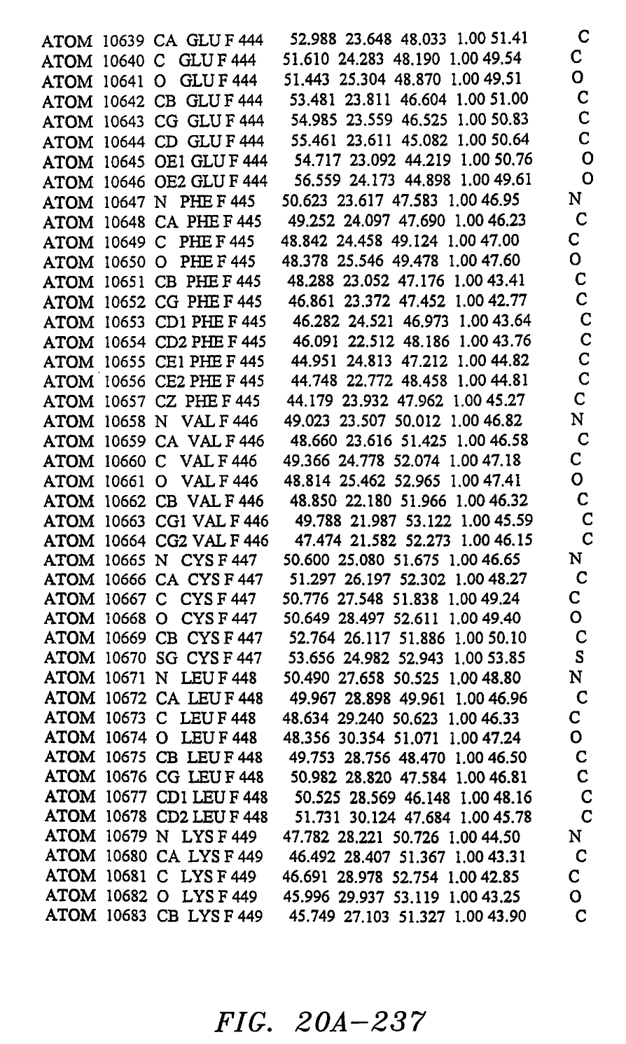

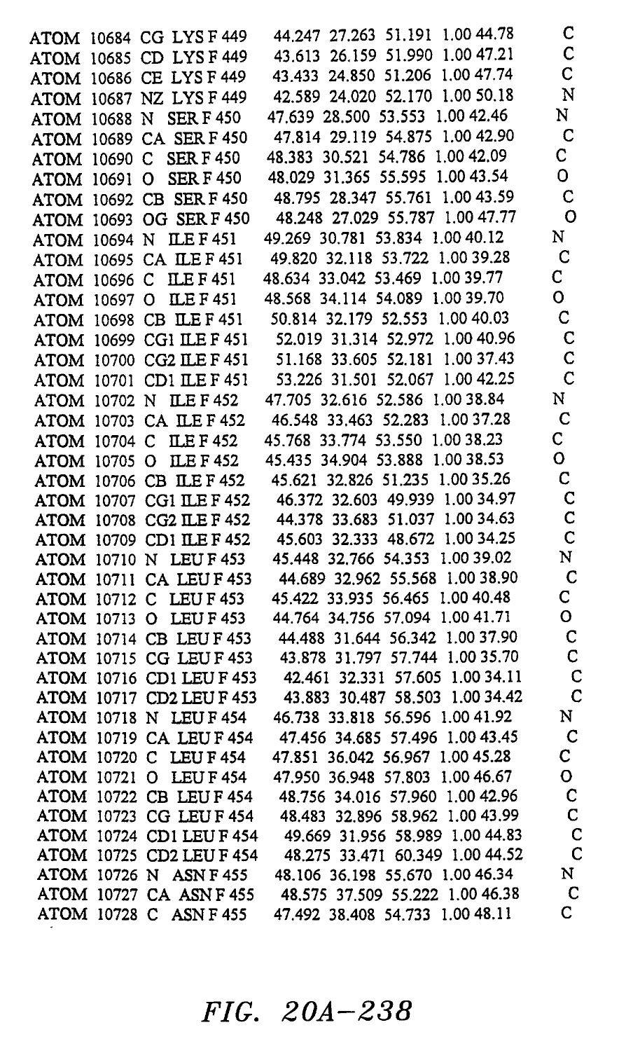

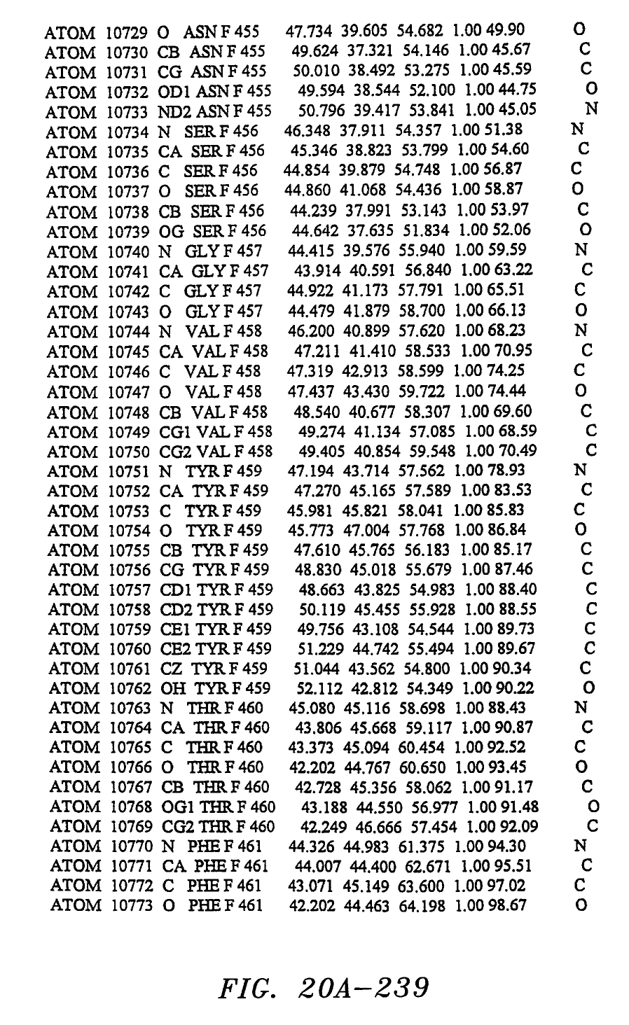

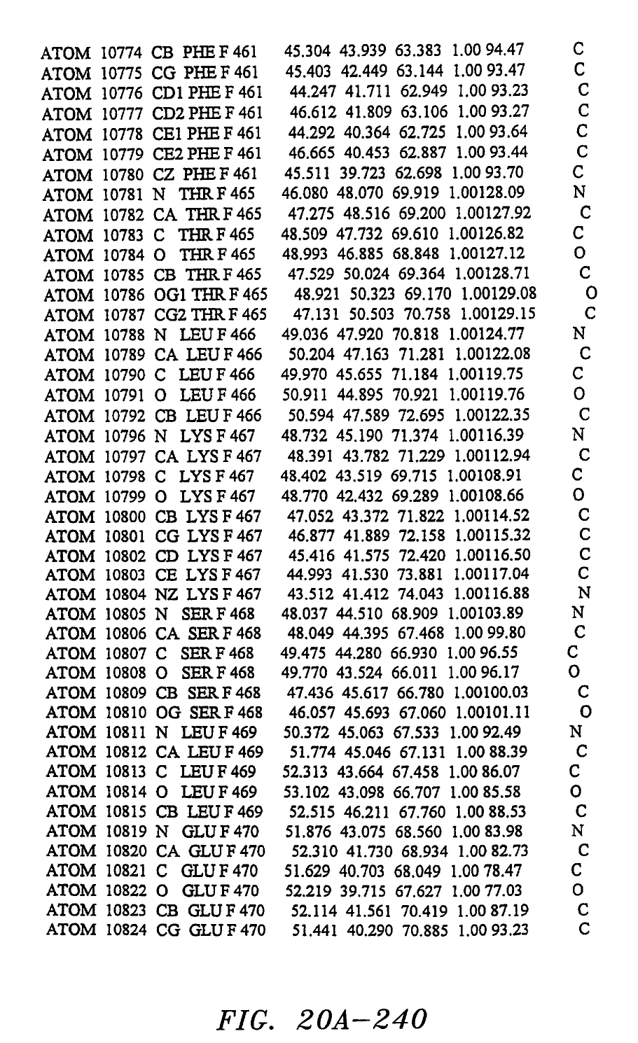

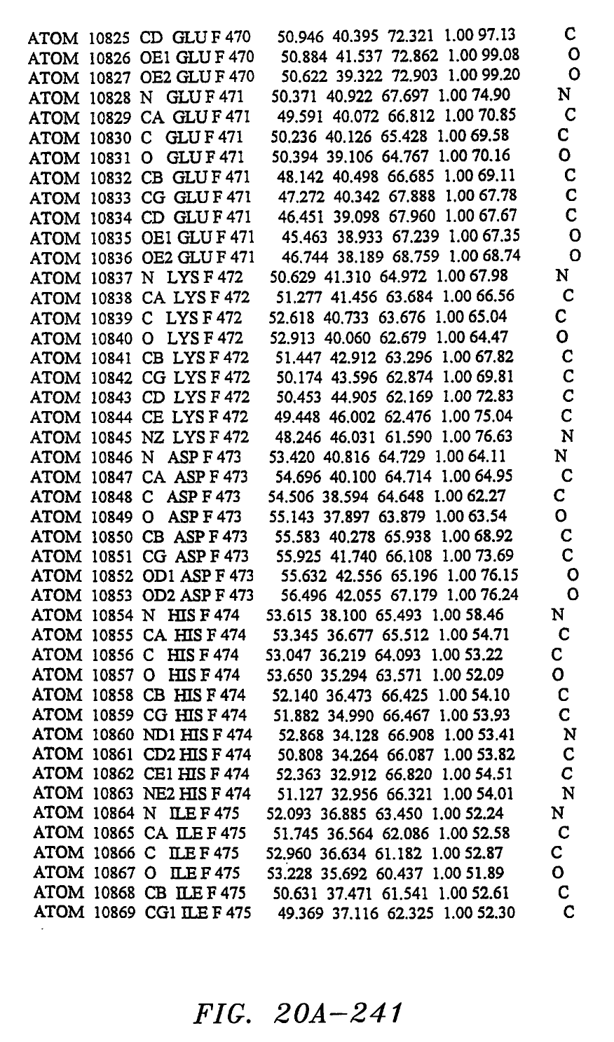

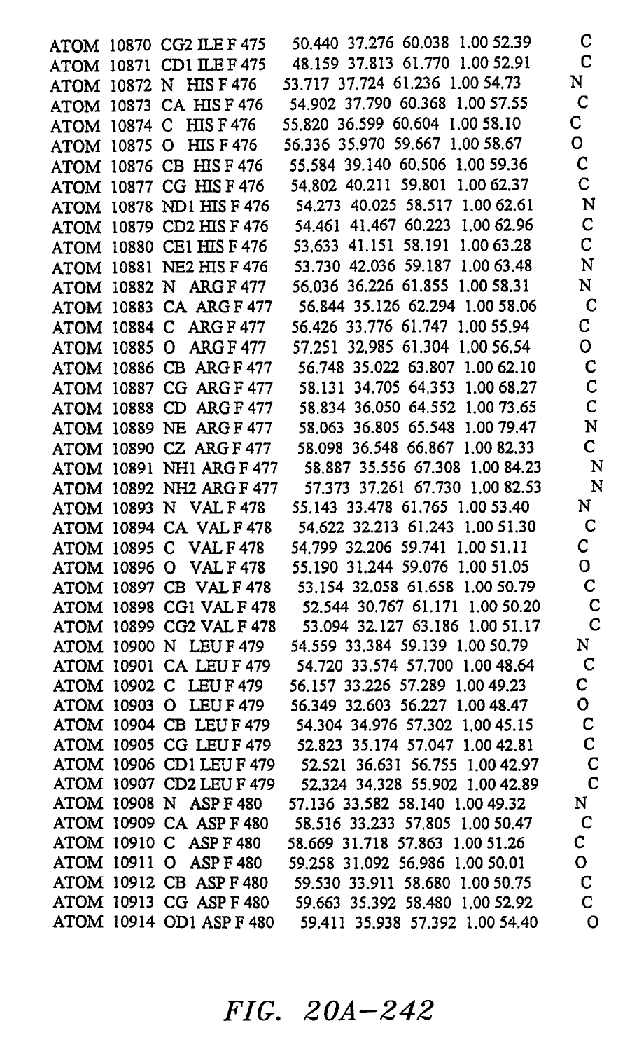

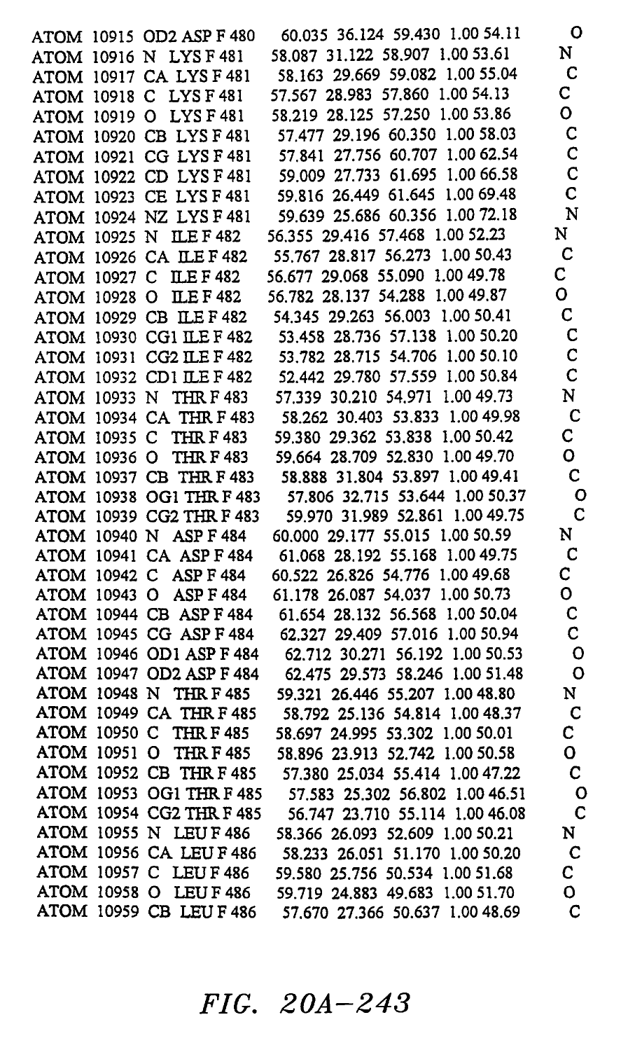

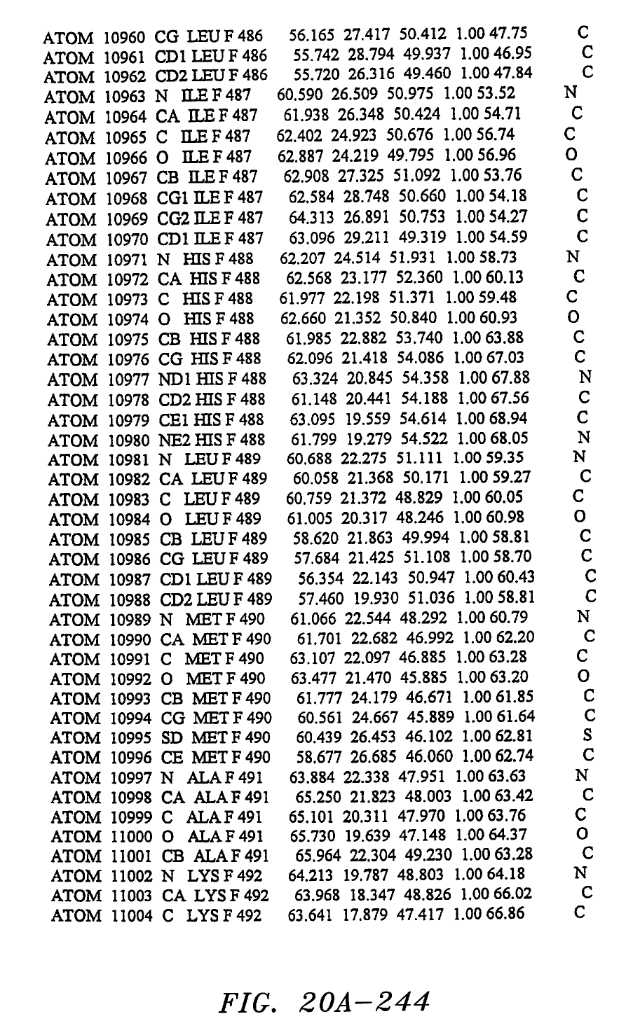

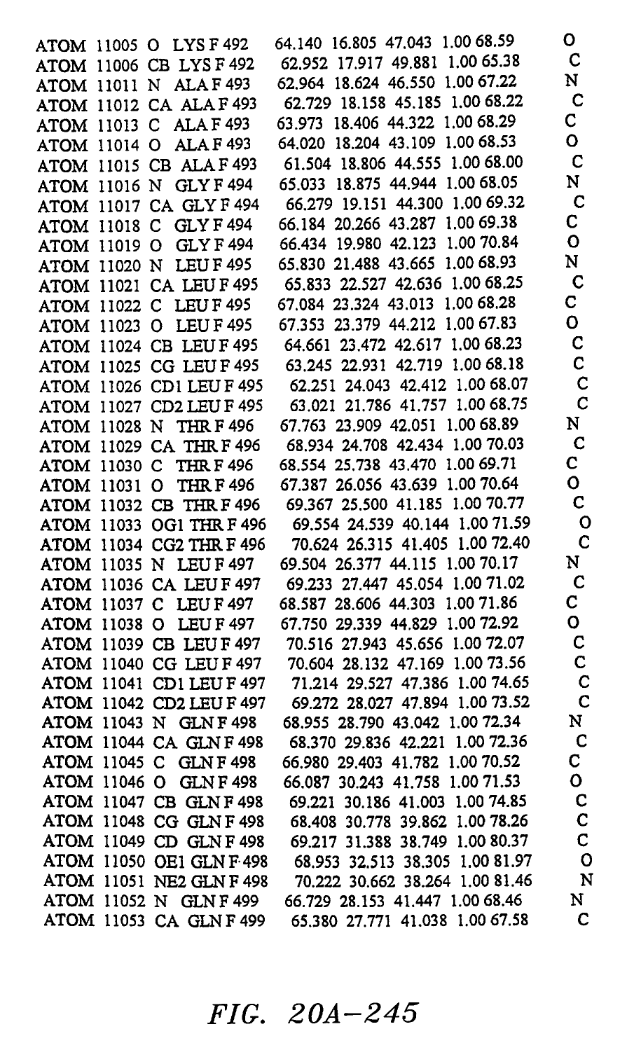

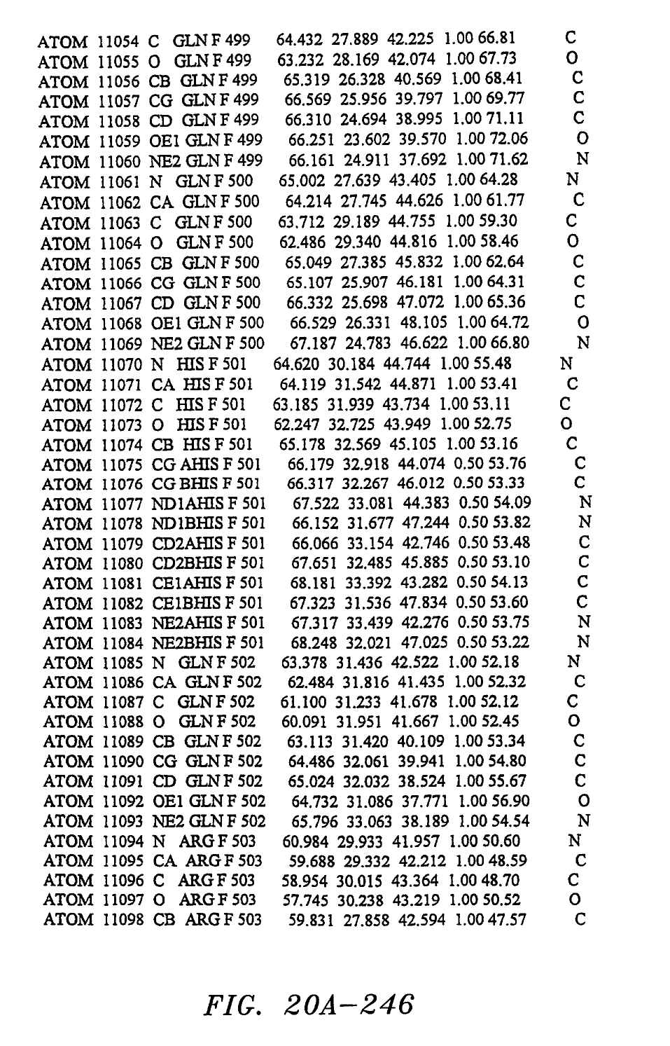

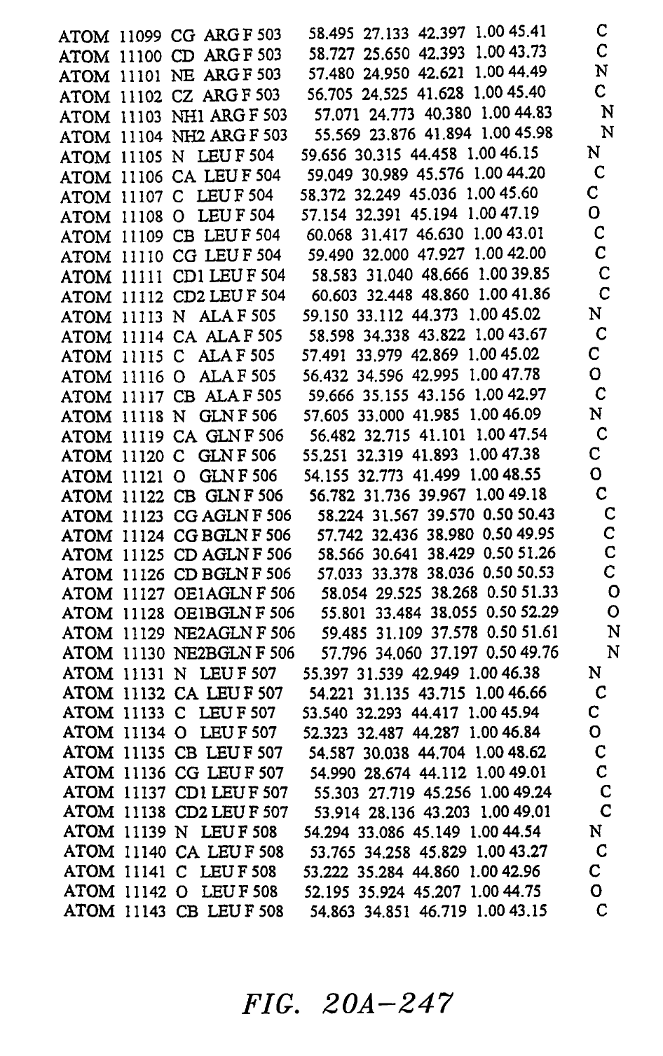

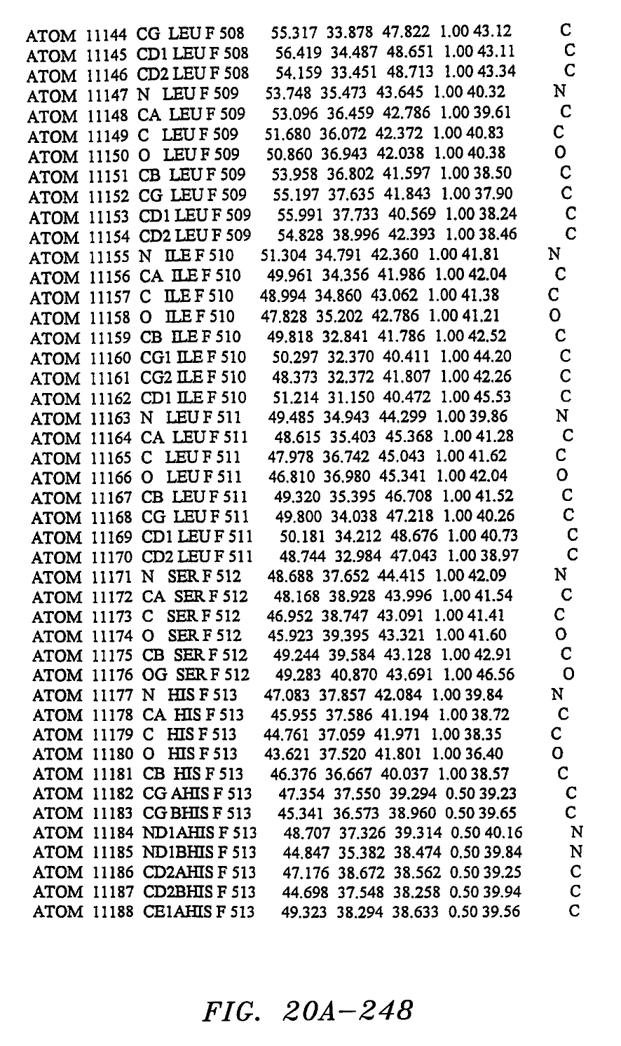

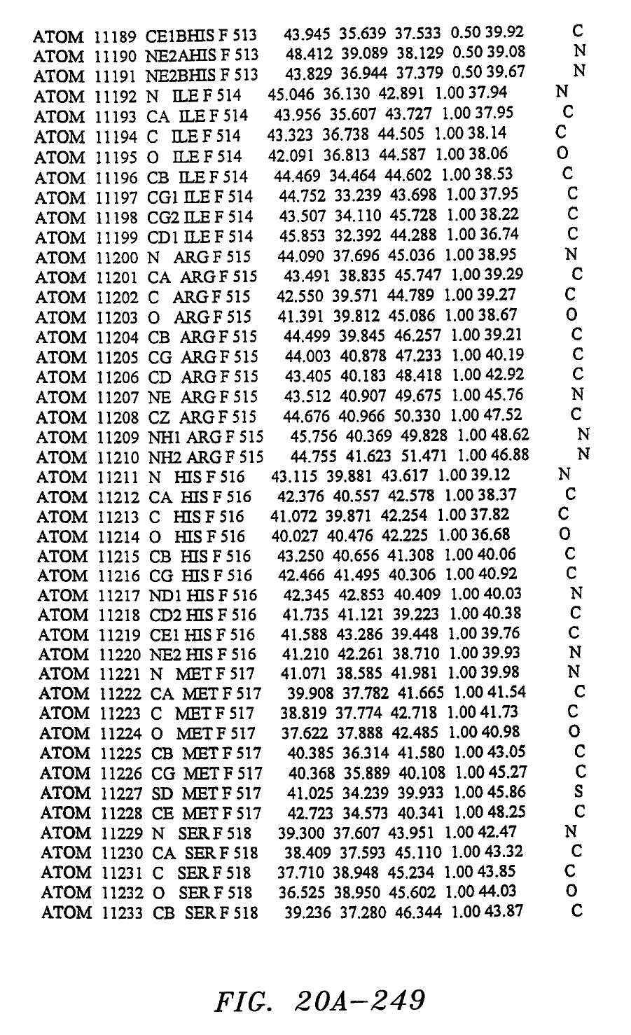

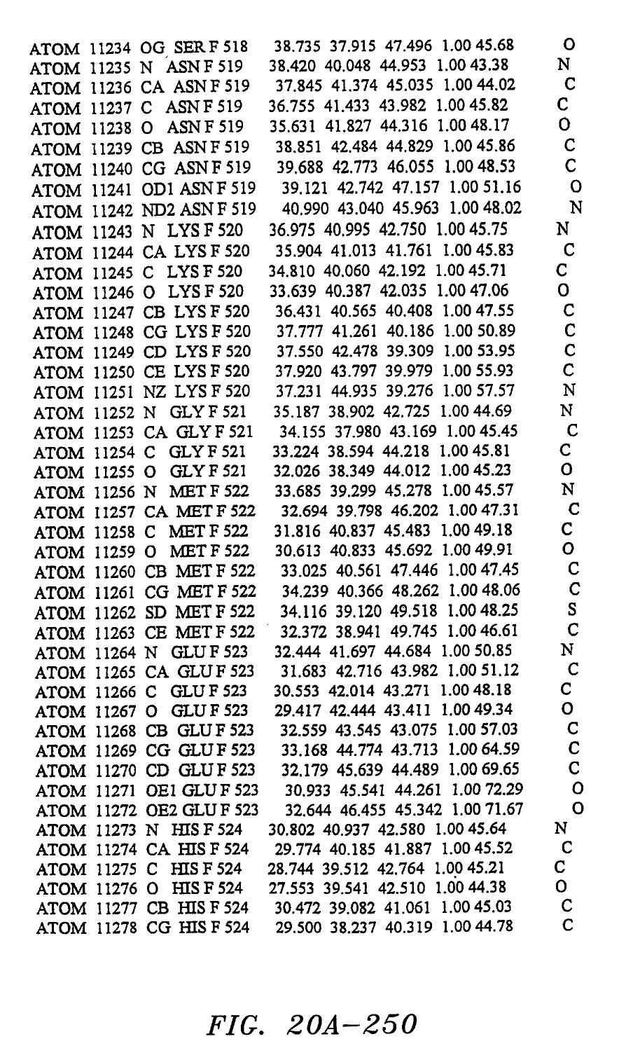

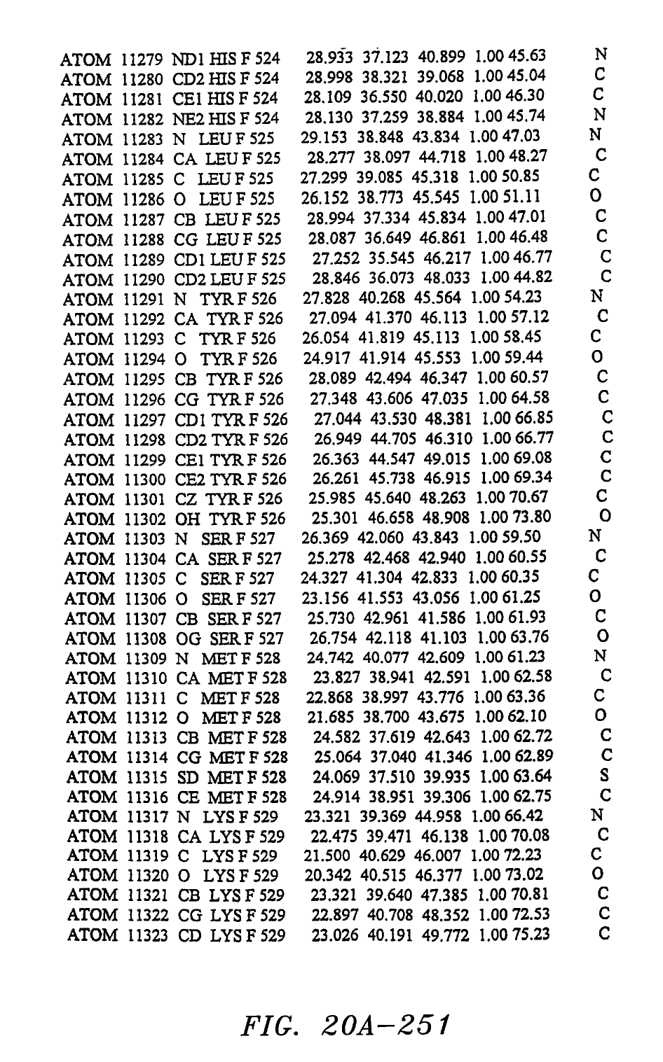

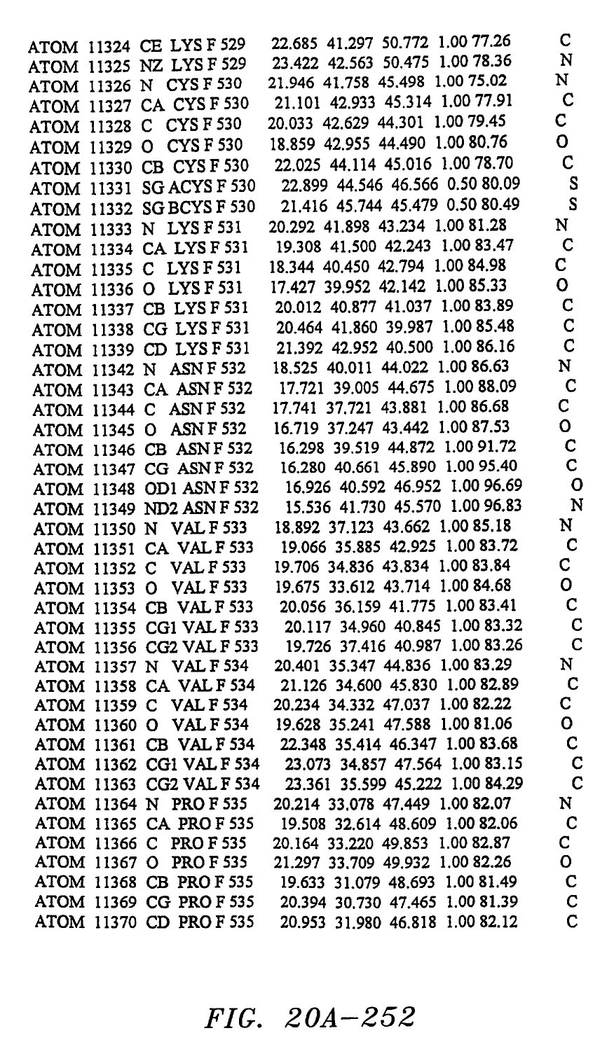

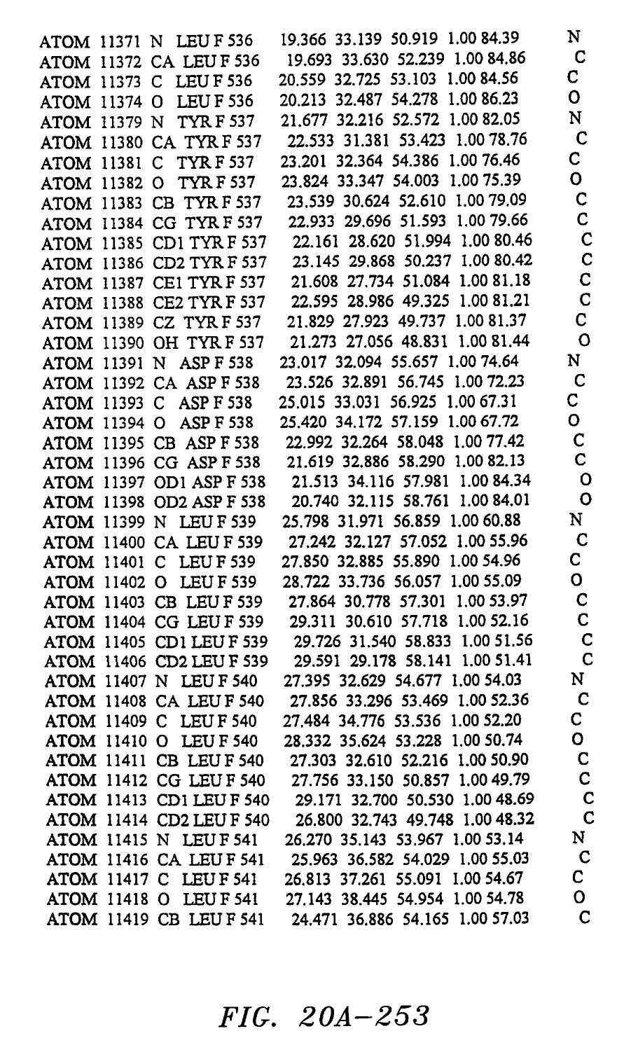

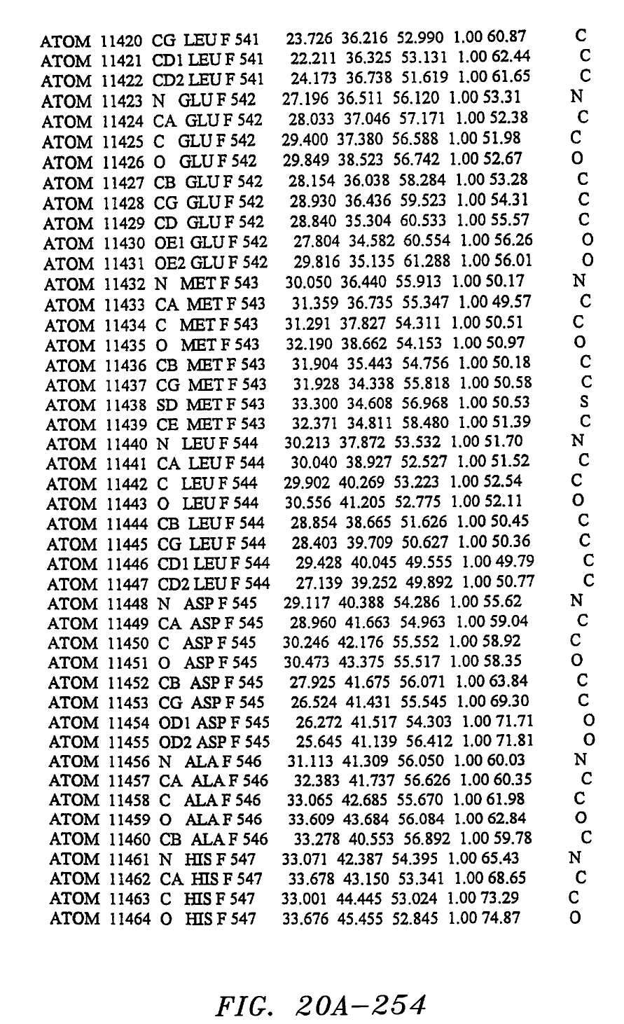

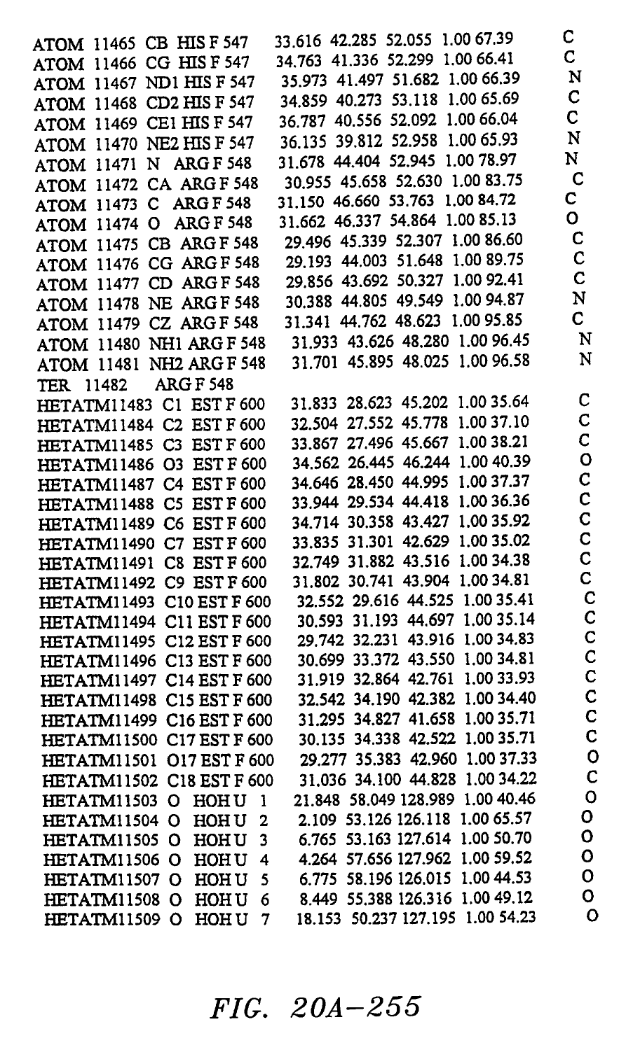

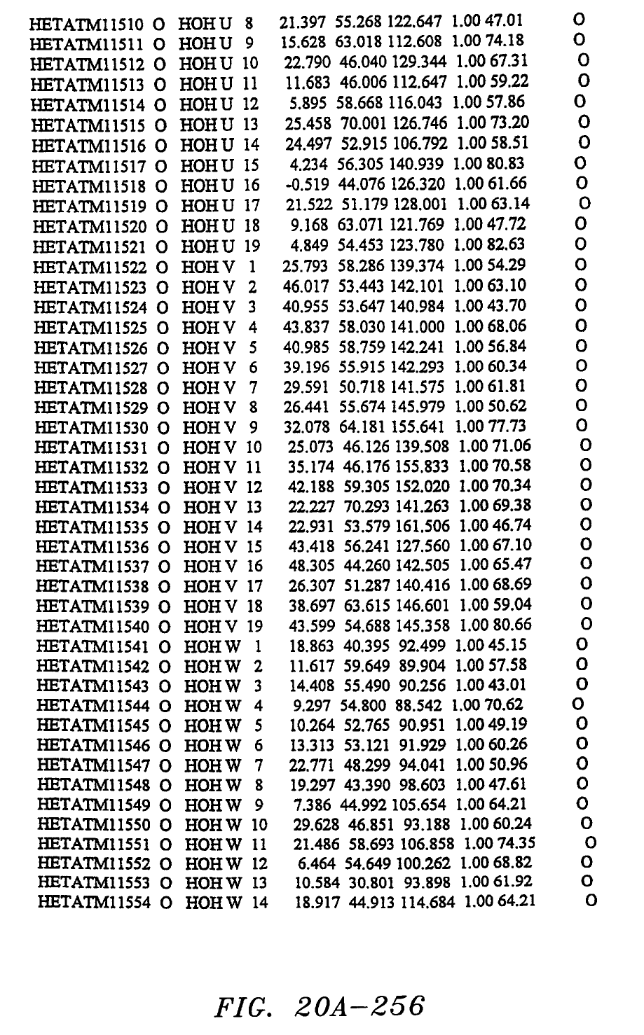

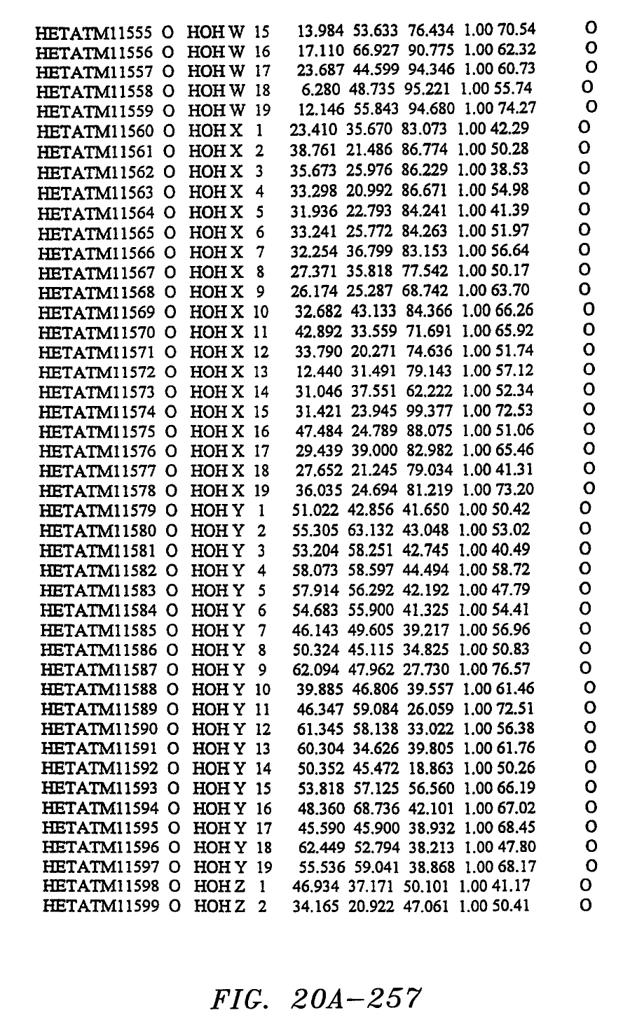

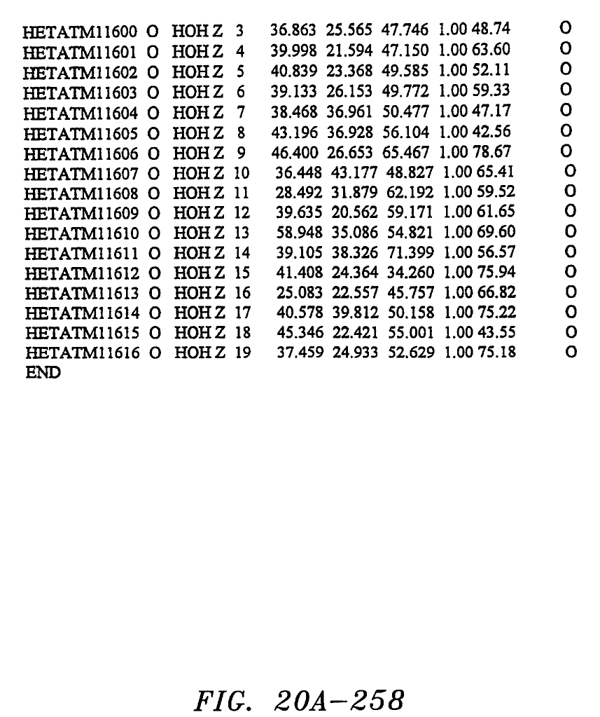

FIG. 20 shows crystal coordinates for estrogen receptor alpha (ERα) binding domain in complex with raloxifene.

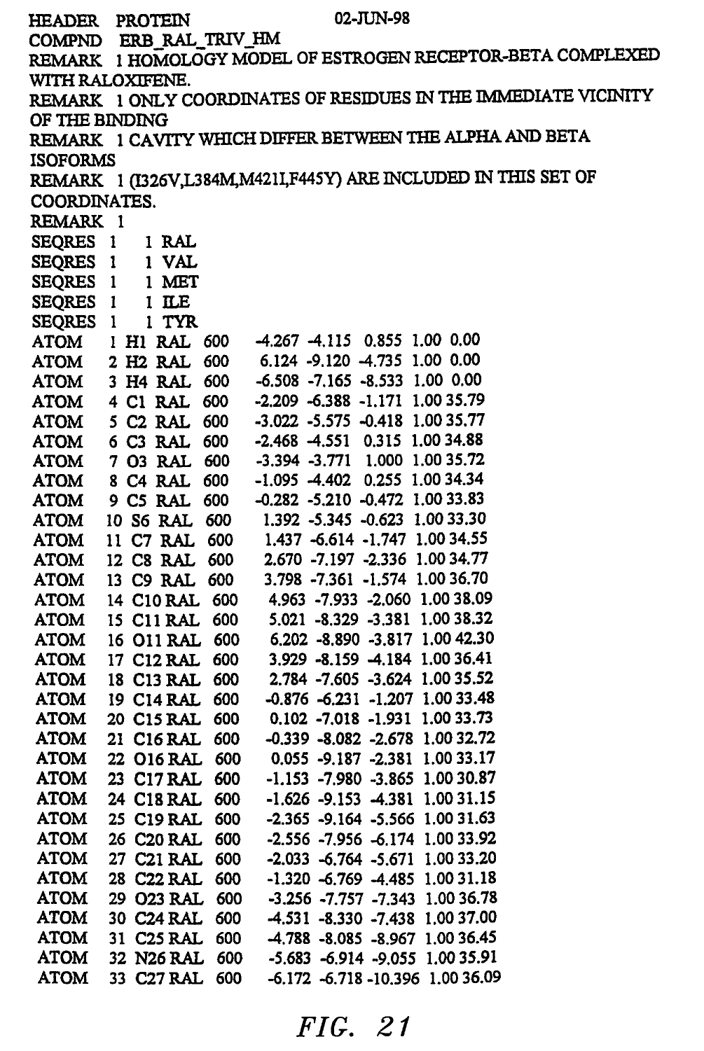

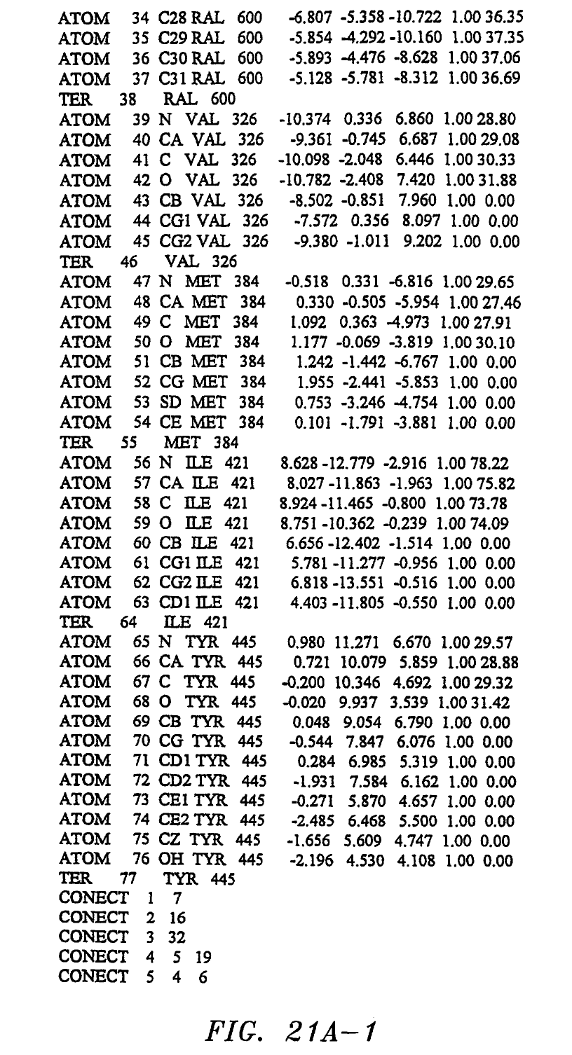

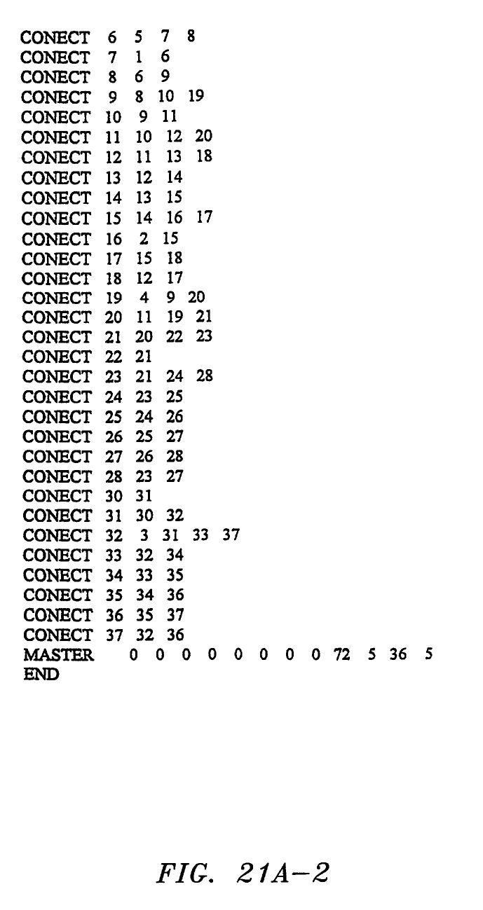

FIG. 21 shows crystal coordinates for estrogen receptor alpha (ERα) binding domain in complex with 17-beta-estradiol.

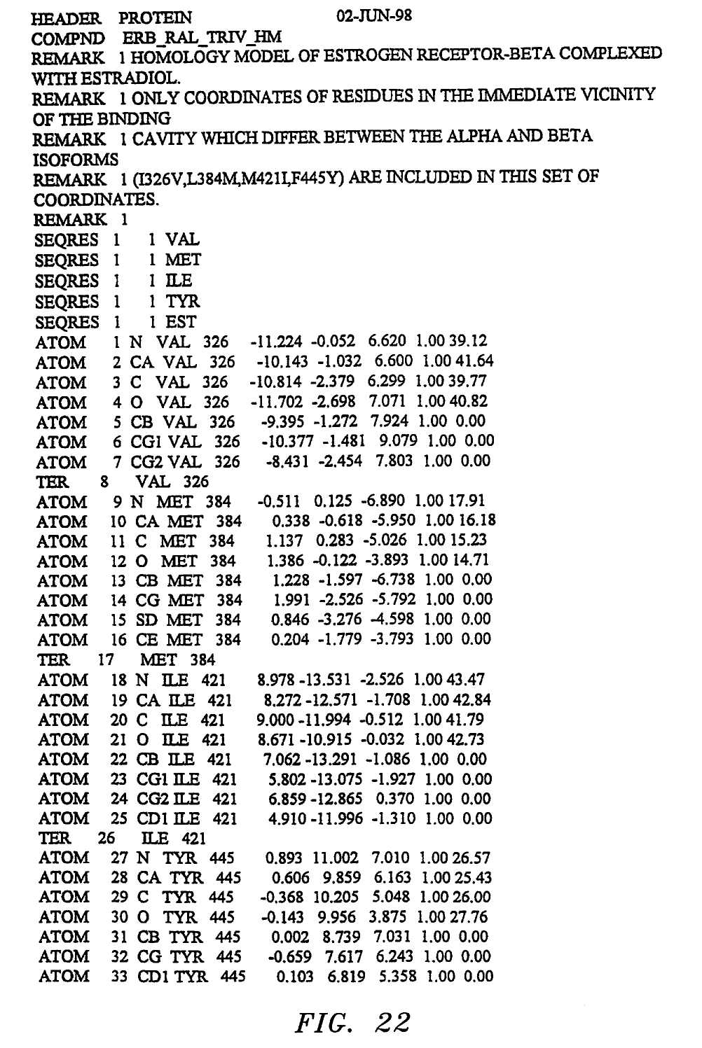

FIG. 22 shows a homology model of estrogen receptor alpha (ERα) beta complexed with raloxifene.

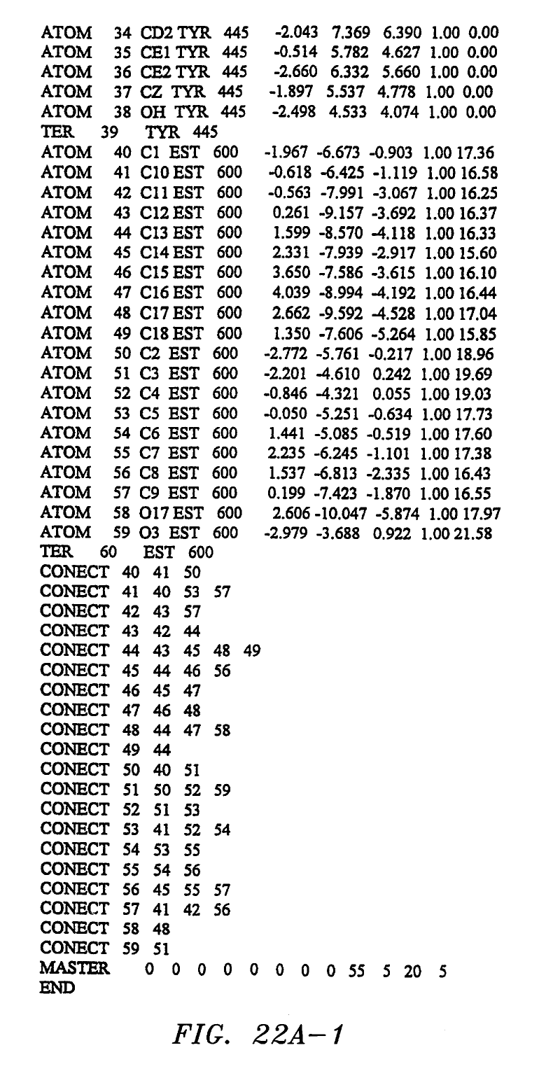

FIG. 23 shows a homology model of estrogen receptor-beta (ERβ) complexed with estradiol.

### EXAMPLE 1

Materials

Protein Purification and Crystallisation of the Oestrogen Receptor α

The human EP-LBD-α was over expressed in Escherichia coli. (Hegy G. B. et al Steroids (1961 61 June 367-373). Fermentation was carried out as batch and fed batch cultivation in a defined glycerol/salt medium at 30° C. Production of recombinant protein was induced by raising the temperature to 39° C. After 2 h, cells were harvested by centrifugation, and frozen, thawed cells were disrupted by a Bead Beater homogenizer (6×22 sec., with a 3 min resting time between bursts) (Biospec. Bartlesville, Okla., USA), at 0° C., in 100 mM Tris-HCl (pH 7.8), 100 mM KCl, 10% glycerol, 4 mM EDTA, 4 mM DTT, 5 μg/ml antipan. For a fermentation volume of 1200 ml, 250 ml buffer was used with 210 ml acid washed glass beads (212-300 microns). After centrifugation, the supernatant was applied to a column of estradiol-Sepharose Fast Flow, 25 ml, (Greene G. et al Proc Natl Acad Sci USA (1980) 77,5115-5119. The column was first washed with 130 ml 10 mM Tris-HCl. (pH 7.8), 700 mM KCl, 1 mM EDTA, followed first by 130 ml 10 mM Tris-HCl (pH 7.8), 250 mM NaSCN, 10% dimethyl-formamide, 1 mM EDTA and then by 110 ml 10 mM Tris-HCl pH 8.0. Reactive Cys residues were modified by washing the column with 120 ml 30 mM Tris-base, 15 mM iodoacetic acid, pH 8.1. Excess reagent was washed out by 50 ml Tris-base, 15 mM iodoacetic acid, pH 8.1. Excess reagents was washed out by 50 ml 10 mM Tris-HCl pH 8.0 followed by 20 ml 10 mM Tris-HCl, ph 7.8, 250 mM NaSCN, 10% dimethylformamide, 1 mM EDTA. The ET-LBD-α was eluted by including 100 μM of the desired ligand to the last buffer. The fractions containing ER-LBD-α was pooled (65 ml) and concentrated (Centriprep 30, Amicon) to 4 ml. Final purification was achieved using a Bio-Rad 491 preparative PAGE instrument according to the user manual. Using one dilution of the Ornstein/Davies buffer system. The stacking (0.7 cm) and resolving (70 cm) gels was 5.6% (acrylamide/bis). The elution buffer was 10 mM Tris-HCl pH 8.0 and the electrophoresis was carried out at 12 W. Fractions containing ER-LBD-α was pooled and concentrated (Centriprep 30) to the desired protein concentration.

Data Collection, Phasing and Refinement

ER-LBD-α-RAL Complex:

A native dataset was collected from a single frozen crystal on beamline X11 at the DESY/Hamburg (l=0.905 Å). Diffraction data were recorded at 120K with a 30 cm Mar Research image plate located at a crystal-to-detector distance of either 245 mm or 390 mm. Heavy atom derivatives were collected in-house (York) from flash frozen crystals. Data were integrated and reduced using the programs DENZO and SCALPACK. MIR analysis was performed using the CCP4 suite of programs (Table 2). Diffraction data for the alternate C2 (York) and C2221 (DESY, Hamburg) crystal forms were collected to resolutions of 3.0 Å and 3.1 Å respectively. Initial phases were calculated to 3 Å using MLPHARE and subsequent two-fold averaging, non-crystallographic matrix refinement and phase extension were carried out using DM. An initial polyalanine trace was used to generate a dimeric search model, using the refined non-crystallographic symmetry and correctly positioned in the alternate C2 and C222 crystal forms using molecular replacement (AmoRe) Collaborative Computational Project No. 4. (The CCP4 Suite: programs for protein crystallography. Acta Cryst D50, 760-763 (1994)). Twenty cycles of cross crystal averaging between all three crystal forms was carried out with DMMULTI (Supra and Cowtan, K, dm: An automated procedure for phase improvement by density modification. In Joint CCP4 and ESF-EACBM Newsletter on Protein Crystallography 31 PP 34-38 (1994)) using only the MIR phase information. The resultant electron density maps showed no bias towards the input model and enabled the unambiguous tracing of the remainder of the molecule and the assignment of most of the amino acid sequence. Refinement was performed with REFMAC using bulk solvent and anisotropic scaling (.Murshudou et al Acta Cryst D53, 240-255 (1997)). Tight non-crystallographic restraints were maintained during the initial cycles but were loosened in the final stages of refinement. Individual atomic temperature factors were refined isotropically. Residues Asp332, Phe337, Lys416, Lys467, Ser468, Leu469, Glu470 and Glu471 were poorly resolved in the electron density maps and not modelled beyond their C $ atoms.

ER-LBD-α-E2 Complex:

Diffraction data were collected at room temperature from a single ER-LBD-E2 crystal using an 128 cm Mar Research image plate located at a crystal-to-detector distance of 280 mm on beamline BW7AB at the DESY/Hamburg (l=0.916 Å). Initial phase estimates were obtained with AMoRe using the refined ER-LBD RAL dimer (truncated after Met528) as a search model. All data between 15 and 4 Å were used for the rotation and translation functions and in the cross-rotation function model Patterson self-vectors were selected within a radius of 30 Å. The correct solution, corresponding to three ER-LBD dimers, had a correlation coefficient of 69.8 and an R-factor of 40.6 after AMoRe rigid-body refinement. Six-fold averaging was performed using DM and the structure was refined with REFMAC using tight non-crystallographic restraints, bulk solvent and anisotropic scaling and averaged phases from DM. A single, overall B value was applied in the early stages of refinement until the Rfree converged. Subsequent cycles employed tightly constrained, full isotropic B value refinement. All model building was carried out using the graphics package QUANTA (Molecular Simulations, Inc. San Diego). The sidechains of Leu306, Leu466, Leu 469, Lys492, Lys531 and Leu346 were poorly resolved in the electron density maps and not modelled beyond their C $ atoms.

TABLE 1
Data collection and refinement statistics

| | ER-raloxifene | ER-estradiol |
|---|---|---|
| Space group | C2 | P21 |
| Unit cell dimensions | | |
| a (Å) | 104.53 | 61.48 |
| B (Å) | 53.68 | 115.16 |
| C (Å) | 102.71 | 137.38 |
| β (o) | 116.79 | 103.01 |
| No. of molecules/AU | 2 | 6 |
| Resolution (Å) | 25-2.6 | 20-3.1 |
| No. unique reflections | 15,497 | 34,025 |
| Completeness (%) | 94.6 | 99.1 |
| Multiplicity | 4.5 | 2 |
| Rsym (I) | 8 | 10 |
| Reflections used in | 13,868 | 30,583 |
| refinement | | |
| Rcryst | 23.98 | 22 |
| Rfree | 30.4 | 25.3 |
| Non H atoms | 3,741 | 11,508 |
| Water | 66 | 126 |
| % A. B, L (a, b, l, p) | 92.4 | 93.0 |
| | (7.6) | (7.0) |
| Rmsd bond length (Å) | 0.01 | 0.01 |
| Rmsd bond angle (Å) | 0.04 | 0.03 |
| Average B protein (Å2) | 48.3 | 37.8 |
| Rmsd NCS protein (Å) | 0.57 | 0.08 |
| Rmsd NCS B (Å2) | 8.2 | 1.1 |

Rsym (I) = 100 × GₕGᵢ1Gₕᵢ − <I>1/GₕGᵢI_{h.,i} where I is the observed intensity. <I> is the average intensity of multiple observations of symmetry related reflections.
Rcryst = 100 × E11Fₒ1 − 1F_{c}11/E1fₒ1 . . .
Rfree is the same as Rcryst but was calculated using a test set of reflections (10% of the whole dataset) that was excluded from the refinement process.
R.m.s. deviation in bond length and the angle distances from Engh and Huber ideal values
R.m.s. distance between all non-crystallographic symmetry (NCS) related atom positions
R.m.s. difference between all non-crystallographic symmetry (NCS) related atomic temperature factors.

TABLE 2
Heavy atom data collection and MIR statistics

| Dataset | PCMBS-1 | PCMBS-2 | KAuCN |
|---|---|---|---|
| Resolution (Å) | 20 − 3 | 20 − 3 | 20 − 3.6 |
| No. unique | 10,335 | 9,316 | 5,835 |
| reflections | | | |
| Completeness (%) | 97.6 | 89 | 94.2 |
| Multiplicity | 4 | 3.1 | 2.5 |
| Rsym (I) | 8.1 | 9.2 | 7 |
| Conc. reagent | 4 | 4 | 4 |
| (mM) | | | |
| Soak time (days) | 5 | 14 | 2 |
| Resolution (Å) | 20 − 3 | 20 − 3 | 20 − 3.6 |
| Riso | 16.9 | 20.7 | 13.7 |
| No. of sites | 2 | 2 | 1 |
| Cullis R (centric/ | 0.75/0.68 | 0.76/0.66 | 0.90/0.85 |
| acentric) | | | |
| Phasing power | 1.22/1.88 | 1.23/2.02 | 0.71/0.94 |
| (centric/acentric) | | | |
| F.O.M (20 − 3Å) | | 0.67/0.48/0.49 | |
| (centric/acentric/ | | | |
| overall) | | | |

Cullis R = E1E1/G11F_{PH} 1 − 1FP_{P}11 for centric (c) and acentric (a) reflections.
F.O.M = <EP(α)e^{iα}/EP(α) where α is the phase and P(α) is the phase probability distribution.
Phasing power.

### EXAMPLE 2

ER-E2 Crystallisation

Prior to crystallisation protein was buffer exchanged to 20 mM using Tris/HCl buffer at pH 7.8 and concentrated to 12-13 mg/ml. Crystals were grown by vapour diffusion using hanging and sitting drop techniques. The best crystals were obtained using 2.4 M Ammonium formate or 80-90 mM Magnesium formate as precipitants buffered with 0.1 M Tris/HCl buffer. 4 M Ammonium formate or 200 mM Magnesium formate unbuffered stock solutions were used. Magnesium formate stock solution was kept at 4° C. and filtered before use. The optimum pH ranged from 7.9 to 8.3 with the best crystals growing at pH 8.1. Protein concentration in the drop was 8 mg/ml although X-ray suitable crystals were also obtained at 13 mg/ml. However, crystals obtained from such conditions were very often twinned and the addition of DMSO at up to 8% significantly improved their quality. The size of the crystals was correlated with the size of the sitting/hanging drop. The optimum size of the drop was achieved by mixing 2.5 ml of protein with 2.5 ml of the reserved solution. All crystallisations were performed at 18° C. The best crystals, with a size of 0.5×0.05×0.05 mm³, were mounted in the X-ray quartz capillaries.

ER-α-Raloxifen (ER-R) Crystallisation

After purification as before, the protein buffer was replaced with 20 mMTris/HCl pH 7.8-7.9 and the protein was concentrated usually to 10-12 mg/ml. The vapour diffusion method with the hanging drop technique was used for crystallations. The best conditions for crystallisation used the following medium: 0.1 M Tris/HCl buffer pH 8.3, 12% (w/v) of PEG 4000, 0.1 M Maltose, 50 mM Lysine, 0.2 M MgCl12, 5% dioxane. The concentration of the protein solution used for crystallisation was brought up to 7.3-7.5 mg/ml by dilution with 20 mM Tris/HCl buffer pH 8.3. Cyrstallisations were performed with different drop sizes and protein-to-reservoir buffer ratios. The best crystals were grown from drops obtained by mixing 2 ml of protein with 2 ml or 3 ml of well buffer. The best temperature for crystal growth is 18° C. These conditions yielded the main C2 crystal form (a=104.53 Å b=53.68 Å c=102.71 Å b=116.79 Å, in the shape of monoclinic plates (0.1×0.1×0.02 mm³)), which was subsequently used for heavy atoms derivatives searches and structure refinement.

By subtle manipulation of the above conditions other crystal forms were also produced. The lowering of the PEG 4000 concentration to 10-11% w/v resulted in other C2 crystal form: a=89.9 Å b=75.09 Å c=87.50 Å b=103.01 o. These crystals grow at 18° C. as single pyramids and despite their severe twinning it was possible to separate mechanically small untwinned fragments of the crystals suitable for X-ray data collection.

The alteration of other conditions, for example increase of dioxan concentration from 5 to 7.5 and 10% and replacement of 50 mM Lys by 50 mM Arg, 0.1 M Maltose by 0.1 M Sucrose or Glucose, produced C2221 orthorhombic crystal form: a=65.47 Å b=95.99 Å c=164.14 Å Crystals reached the size of 0.2×0.03×0.03 mm3 and they were growing preferably at 18° C.

It is also possible to obtain crystals of SeMet derivative or ER-R complex. They can be grown from conditions typical for the main C2 crystal form, but the concentration of dioxan is raised usually up to 7.5%. These crystals were very fragile and give poor quality X-ray data: which was used as additional information for positioning Met residues only.

All ER-RAL complex crystal forms were suitable for flash-cooling by using a stream of N₂ at 100 K and 120 K. In all cases, the cryoprotectant consisted of mother liquor (well buffer composition) and 25% v/v MPD.

Owing to the sensitivity of the ER-R crystals all heavy atoms soaks were done in the exact mother liquor (taken from the well buffer) and the heavy atoms compounds were always dissolved as a solid substance in these solutions. PCMBS-1 and KAuCN soaks were done for three days, PCMBS-2 for three weeks. All soaks done at 18° C. The cryo-solutions contained the heavy atom compounds at soaking concentration as well.

Pure ER-LBD is particularly refractive to crystallisation and suitable crystals were obtained after carboxymethylation of the three thiol groups.

### EXAMPLE 3

Structure Determination of the Estrogen Receptor α-Ligand Binding Domain

Crystals of the ER “-LBD complexed with either estradiol or raloxifen will diffract to medium resolution, are monoclinic and contain either a single dimer in the case of raloxifen or three dimers in the case of estrogen in the asymmetric unit (see Table 1). Multiple isomorphous replacement was used to determine the crystal structure of the ER-LBD-RAL complex. An initial multiple isomorphous replacement/density modified electron density map showed the position of the non-crystallographic two-fold rotation axis and allowed an initial polyalanine trace to be built on the resultant two-fold averaged map. Subsequent averaging between three different crystal forms of the RAL complex enabled corrections to be made to the initial trace. The remainder of the protein as yet not being unambiguously traced and most of the amino acid sequence to be assigned. The resultant model had an R value of 43%. Cycles of maximum likelihood refinement and manual rebuilding yielded a final model with acceptable R values and geometric parameters. The initial phase estimates were obtained for the estradiol (E2) complex by molecular replacement using the ER-LBD RAL dimer as a search model. Rotation in translation functions yielded the correct solution. Six-fold averaging between the three dimers in the crystal line asymmetric unit allowed both the missing parts of the structure to be traced and the positioning of E2 in the binding cavity to be determined. The structure was refined using both tight non-crystallographic restraints as well as average phase information to yield a final model with an Rcryst of 22.0 and R3 of 25.3 for all data between 20 and 3.1 \mathring{6} (Table 1).

Results

The crystals produced in Example I and II were subjected to X-ray crystallographic studies which revealed that the LBD is folded into a characteristic “wedge-shaped” globular unit. It has a three-layered, anti-parallel a-helical sandwich motif and is constructed from 8 major helices The motif comprises a central core layer of 3 helices (H5/6, H9 and H10) sandwiched between two additional layers of helices (H2-3 and H7, H8, H11). The arrangement of structural elements in this fashion creates a “molecular scaffold” maintaining a sizable ligand binding cavity at the “toe end” of the wedge-shaped domain. The remaining secondary structural elements, a small two stranded anti-parallel β-sheet (S1 and S2) and helix H12, are located at the “ligand binding end” of the molecule and flank the main three-layered motif (see FIG. 2). From the N-terminus, the chain follows one turn of the distorted “-helix (H1), turns 90° and enters a short helix (H2) that lies parallel to the longest axis of the LBD. After helix H2, the chain continues in the same direction in an irregular extended conformation before tucking under the bottom of the molecule. At this stage, the chain turns back on itself through the long, bent, helix (H3). The N-terminal portion of this helix forms part of the ligand binding cavity. The sequence has a proline at position 365 which is invariable and it is at this residue that the main chain takes a sharp (90°) change in direction, passes through a 310 helix (H4) before forming the first of three central helices (H5/6). Helix H5/6 can be geometrically described as a single unit, although it is kinked by 40° at the alanine residue at position 382 in a manner such that its C-terminal end is correctly positioned to form part of the E2 binding cavity. This helix is kinked and is distinguishing and is maintained by a series of hydrophobic interactions between leucines at 378 and 379 (H5) with a phenol at 367 and leucine at 453 all of which are highly conserved and are part of the nuclear receptor LBD signature motif (Wurst). From this position the sequence passes through a small $ \ hairpin (S1/S2) covering one side of the binding cavity, and emerges on the other side of the molecule via the 3₁₀ helix H7. Helix H8 runs three quarters of the way up the long axis of the LBD, passes through a second central helix (H9) before turning back via a disordered loop to form a final helix H10. At the end of H10, the polypeptide backbone changes direction and runs the full length of the ligand binding domain, in an anti-parallel direction to H8 in the form of helix 11. After a short turn the chain emerges on the opposite side to the S1/S2 β hairpin at helix H12, the core amphipathic helix of the AF-2 region.

Dimerisation

Crystallographic studies also reveal that the receptor is dimerised. ER is sequestered in an inactive complex with heat shock protein 90 (hsp90) and other accessory factors in the absence of E2. Ligand binding initiates the disassembly of this complex and results in receptor dimerisation via domain E. The ligand-bound form of ER exists as a tight homodimer in solution and ER-LBDs are arranged as non-crystallographic dimers within both the E2 and RAL complex crystals. This quaternary arrangement almost certainly reflects the physiological state of ER-LBD in vivo as all crystal forms of the liganded ER-LBD obtained to date contained non-crystallographic dimers. The dimer axis coincides with the longest axis of the LBD with each molecule tilted approximately 15° away from the two position fold. This symmetric arrangement generates a molecule with dimensions of approximately 55 Å high by 50 Å wide by 35-60 Å breadth. The observed quaternary arrangement locates the N and C termini of each monomer on the opposite “faces” of the dimer. The C terminus of each monomer projects towards the dimer axis while the N termini are far removed from the interface. The dimerisation surface is fairly extensive and encompasses about 15% (1,703 Å²) of each monomer's accessible surface area. The LBD's are positioned so that the H8/H11 face of each monomer lines up to form an additional, intermolecular helical layer. Contacts between the two molecules are made primarily through the H11 helices, which intertwine to form a rigid backbone, but also involve H8 from one monomer and H9 and H10 from the neighbouring monomer. The H11 helices are arranged as a bifurcated coiled coil with the side chains of the residues Leu 504, Ala 505, Leu 508, Leu 509 and Leu 511 which are interdigitated to form a partial “leucine zipper” motif at the coils end terminal N. This hydrophobic patch is flanked on either side by a network of hydrogen bonding residues. Arg 545 and Asn 519 make direct hydrogen bonds with Ser 512 and His 516 respectively. This overall monomer-monomer arrangement is unaffected by the nature of the ligand and seems to be maintained within the receptor super family. The observed ER-LBD dimer is identical in terms of gross monomer orientation and make up of the dimer interface to that of the crystallographic unliganded RXR-α homodimer (58% hydrophobic/42% hydrophillic).

The invariable nature of the LBD's quaternary structure therefore suggests that it provides a stable entity that facilitates separation of the two DNA binding domains in such a way as to allow optimal binding to EREs.

Such an elucidation of the 3-dimensional structure of the estrogen receptor ligand binding domain provides a useful tool for designing ligands for binding to the estrogen receptor. Such a detailed knowledge of the structure of the receptor enables prediction with accuracy whether a ligand binding to the receptor will act as an antagonist, a partial antagonist, an agonist or a partial agonist since the ligand-induced conformational changes can be anticipated.

### EXAMPLE 4

Partial Homology Model of ERβ

The coordinates obtained in Example I (ERα complexed with either estadiol or with raloxifine) were used to create two partial homology models of ERβ (complexed with estradiol and raloxifene respectively). This was accomplished by importing the ERα coordinates into version 6.4 of Sybyl (available from Tripos Associates, St. Louis, Mo., U.S.A.). The “change” command in the Sybyl biopolymer module was used to mutate amino acids which differ between ERα and ERβ and which are in the vicinity of the ligand binding pocket. Four such residues were mutated: 1326V (Ile-326 to Val), L384M (Leu-384 to Met), M421I (Met-421 to Ile), F445Y (Phe-445 to Tyr). These partial ERβ homology models in conjunction with the experimental ERα coordinates were used to design isoform selective ligands as described in Example 5-51.

Design of Ligands

Examples of ligands designed to fit the receptor have been produced as follows:

### EXAMPLE 5

2-(2,6-dimethylphenyl)-6-hydroxybenzo[β]thiophene (1).

(a) To a solution of 6-methoxybenzo[β]thiophene (Graham et al, J. Med. Chem., 1989, 32, 2548.) (6 g, 36.5 mmol) in 50 ml of anhydrous tetrahydrofuran at −60° C. was added n-butyllithium (20.5 ml, 41 mmol, 2.0 M solution in cyclohexane), dropwise via a dropping funnel. After stirring for 30 minutes, trimethyltin chloride (41 ml, 41 mmol, 1.0 M solution in hexanes) was introduced dropwise through a dropping funnel. The resulting mixture was allowed to warm to 0° C., stirred for 1 hour and then quenched with 100 ml of 1 M hydrochloric acid. The aqueous phase was extracted with ethyl acetate. The combined organic phases were dried over sodium sulphate and then concentrated in vacuo. This produced 9.24 g (28 mmol, 77%) of 2-trimethylstannyl-6-methoxybenzo[β]thiophene as white semicrystals. ¹H NMR (CDCl₃) 7.66 (d, J=8.6 Hz, 1H), 7.34 (d, J=2.2 Hz, 1H), 7.29 (s, 1H), 6.95 (dd, J=8.6 Hz, 2.2 Hz, 1H), 3.86 (s, 3H), 0.39 (s, 9H).

(b) A mixture of 370 mg (2 mmol) 2-bromo-m-xylene, 115 mg (0.1 mmol) tetrakis triphenylphosphinepalladium (0) and 160 mg (2 mmol) of cupric oxide in 8 ml of N,N-dimethylformamide was stirred at 100° C. under nitrogen. After 5 minutes, 981 mg (3 mmol) of 2-trimethylstannyl-6-methoxybenzo[β]thiophene (example 1a) in 2 ml of N,N-dimethylformamide was added all at once to the reaction mixture. The reaction was heated for 2 hours and then allowed to reach room temperature. The resulting mixture was concentrated, dissolved in ethylacetate, filtered through a pad of silica and concentrated. The crude product was purified on a chromatotron (silica, 99:1, petroleum ether/ethyl acetate) producing 328 mg (1.22 mmol, 61%) of 2-(2,6-dimethylphenyl)-6-methoxybenzo[β]thiophene a yellowish crystals. ¹H NMR (CDCl₃) 7.89 (d, J=8.7 Hz, 1H), 7.32-7.59 (m, 4H), 7.23 (dd, J=8.7 Hz, 2.2 Hz, 1H), 7.18 (s, 1H), 4.12 (s, 3H), 2.46 (s, 6H).

(c) 145 mg (0.54 mmol) of 2-(2,6-dimethylphenyl)-6-methoxybenzo[β]thiophene (example 1b) was dissolved in 15 ml of dichloromethane, to the stirred solution was added boron trifluoride dimethylsulfide complex (1.5 ml). The solution was stirred at room temperature under nitrogen in the dark for 15 hours. The reaction mixture was quenched with 10 ml of water, extracted with dichloromethane, dried over magnesium sulphate and concentrated. The crude product was purified on a chromatotron (silica, 80:20, petroleum ether/ethyl acetate) producing 94.1 mg (0.37 mmol, 69%) of 2-(2,6-dimethylphenyl)-6-hydroxybenzo[β]thiophene as white crystals. MP 95-96° C. ¹H NMR (CDCl₃) 7.63 (d, J=8.6, 1H), 7.08-7.31 (m, 4H), 6.92 (s, 1H), 6.91 (dd, J=8.6, 2.2 Hz, 1H), 4.91 (s, 1H), 2.20 (s, 6H).

### EXAMPLE 6

2-(2-ethyl-6-methylphenyl)-6-hydroxybenzo[β]thiophene (2).

(a) The cross-coupling of 492 mg (2 mmol) 2-ethyl-6-methyliodobenzene, with 981 mg (3 mmol) of the product from 1(a) was accomplished by the procedure set forth in example 1(b). The crude product was purified on a chromatotron (silica, 99:1, petroleum ether/ethyl acetate) producing 438 mg (1.55 mmol, 78%) of 2-(2-ethyl-6-methylphenyl)-6-methoxybenzo[β]thiophene as a colourless oil. ¹H NMR (CDCl₃) 7.67 (d, J=8.9 Hz, 1H), 7.08-7.36 (m, 4H), 7.01 (dd, J=8.9 Hz, 2.2 Hz 1H), 6.96 (s, 1H), 3.89 (s, 3H), 2.54 (q, J=7.6 Hz, 2H), 2.19 (s, 3H), 1.12 (t, J=7.6 Hz, 3H).

(b) The deprotection of 100 mg (0.35 mmol) of 2-(2-ethyl-6-methylphenyl)-6-methoxybenzo[β]thiophene (example 2(a)) was accomplished by the procedure set forth in example 1(c). The crude product was purified on a chromatotron (silica, 90:10, petroleum ether/ethyl acetate) producing 69 mg (0.26 mmol, 73%) of 2-(2-ethyl-6-methylphenyl)-6-hydroxybenzo[β]thiophene as white semicrystals. ¹H NMR (CD₃OD) 7.59 (d, J=8.7, 1H), 7.06-7.25 (m, 4H), 6.90 (s, 1H), 6.88 (dd, J=8.7, 2.2 Hz, 1H), 2.51 (q, J=7.6 Hz, 2H), 2.15 (s, 3H), 1.09 (t, J=7.6 Hz, 3H).

### EXAMPLE 7

2-(2.6-dimethyl-4-hydroxyphenyl)-6-hydroxybenzo [β]thiophene (3).

(a) The cross-coupling of 402 mg (2 mmol) 4-bromo-3,5-dimethylphenol, with 981 mg (3 mmol) of the product from 1(a) was accomplished by the procedure set forth in example 1(b). The crude product was purified on a chromatotron (silica, 90:10, petroleum ether/ethyl acetate) producing 210 mg (0.74 mmol, 37%) of 2-(2,6-dimethyl-4-hydroxyphenyl)-6-methoxybenzo[β]thiophene as yellow crystals. ¹H NMR (CDCl₃) 7.88 (d, J=8.7 Hz, 1H), 7.55 (d, J=2.5 Hz, 1H), 7.22 (dd, J=8.7 Hz, 2.5 Hz 1H), 7.14 (s. 1H), 6.83 (s, 2H), 4.94 (s, 1H), 4.11 (s, 3H), 2.38 (s, 6H).

(b) The deprotection of 100 mg (0.35 mmol) of 2-(2.6-dimethyl-4-hydroxyphenyl)-6-methoxybenzo[β]thiophene (example 3(a)) was accomplished by the procedure set forth in example 1(c). The crude product was purified on a chromatotron (silica, 80:20, petroleum ether/ethyl acetate) producing 52 mg (0.19 mmol, 54%) of 2-(2,6-dimethyl-4-hydroxyphenyl)-6-hydroxybenzo[β]thiophene as white crystals. MP 202-204° C., ¹H NMR (CD₃OD) 7.56 (d, J=8.7, 1H), 7.19 (d, J=2.2 Hz, 1H), 6.86 (dd, J=8.7, 2.2 Hz, 1H), 6.84 (s, 1H), 6.54 (s, 2H), 2.10 (s, 6H).

### EXAMPLE 8

2-(2-methylphenyl)-6-hydroxybenzo[β]thiophene (4).

(a) The cross-coupling of 340 mg (2 mmol) 2-bromotoluene, with 981 mg (3 mmol) of the product from 1(a) was accomplished by the procedure set forth in example 1(b) The crude product was purified on a chromatotron (silica, 99:1, petroleum ether/ethyl acetate) producing 500 mg (1.97 mmol, 98%) of 2-(2-methylphenyl)-6-methoxybenzo[β]thiophene as white crystals. ¹H NMR (CDCl₃) 7.66 (d, J=8.7 Hz, 1H), 7.19-7.49 (m, 5H), 7.15 (s, 1H), 6.99 (dd, J=8.7, 2.3 Hz, 1H), 3.88 (s, 3H), 2.48 (s, 3H).

(b) The deprotection of 125 mg (0.49 mmol) of 2-(2-methylphenyl)-6-methoxybenzo[β]thiophene (example 4(a)) was accomplished by the procedure set forth in example 1(c). The crude product was purified on a chromatotron (silica, 90:10, petroleum ether/ethyl acetate) producing 60 mg (0.23 mmol, 47%) of 2-(2-methylphenyl)-6-hydroxybenzo[β]thiophene as white crystals. MP 97-98° C., ¹H NMR (CDCl₃) 7.63 (d, J=8.4 Hz, 1H), 7.18-7.48 (m, 5H), 7.14 (s, 1H), 6.91 (dd, J=8.4, 2.3 Hz, 1H), 4.86 (s, 1H), 1.56 (s, 3H).

### EXAMPLE 9

2-(2-chloro-6-methylphenyl)-6-hydroxybenzo[β]thiophene (5).

(a) The cross-coupling of 505 mg (2 mmol) 3-chloro-2-iodotoluene, with 981 mg (3 mmol) of product from 1(a) was accomplished by the procedure set forth in example 1(b). The crude product was purified on a chromatotron (silica, 99:1, petroleum ether/ethyl acetate) producing 439 mg (1.52 mmol, 76%) of 2-(2-chloro-6-methylphenyl)-6-methoxybenzo[β]thiophene as a yellow oil. ¹H NMR (CDCl₃) 7.68 (d, J=8.7 Hz, 1H), 7.15-7.36 (m, 4H), 7.03 (s, 1H), 7.01 (dd, J=8.7, 2.2 Hz, 1H), 3.88 (s, 3H), 2.25 (s, 3H).

(b) The deprotection of 100 mg (0.35 mmol of 2-(2-chloro-6-methylphenyl)-6-methoxybenzo[β]thiophene (example 5(a)) was accomplished by the procedure set forth in example 1(c). The crude product was purified on a chromatotron (silica, 90:10, petroleum ether/ethyl acetate) producing 44 mg (0.16 mmol, 46%) of -2-(2-chloro-6-methylphenyl)-6-hydroxybenzo[β]thiophene as a yellowish oil. ¹H NMR (CD₃D) 7.62 (d, J=8.7 Hz, 1H), 7.18-7.35 (m, 4H), 6.99 (s, 1H). 6.89 (dd, J=8.7, 2.2 Hz, 1H), 2.23 (s, 3H).

### EXAMPLE 10

2-(2-methylnaphth-1-yl)-6-hydroxybenzo[β]thiophene (6).

(a) The cross-coupling of 221 mg (1 mmol) 1-bromo-2-methylnaphthalene, with 491 mg (1.5 mmol) of product from 1(a) was accomplished by the procedure set forth in example 1(b). The crude product was purified on a chromatotron (silica, 99:1, petroleum ether/ethyl acetate) producing 159 mg (0.52 mmol, 52%) of 2-(2-methylnaphth-1-yl)-6-methoxybenzo[O]thiophene as white crystals. ¹H NMR (CDCl₃) 7.66-7.88 (m, 4H), 7.30-7.48 (m, 4H), 7.11 (s, 1H), 7.04 (dd, J=8.7, 2.2 Hz, 1H), 3.91 (s, 3H), 2.40 (s, 3H).

(b) The deprotection of 110 mg (0.36 mmol) of 2-(2-methylnaphth-1-yl)-6-methoxybenzo[β]thiophene (example (6a)) was accomplished b the procedure set forth in example 1(c). The crude product was purified on a chromatotron (silica, 90:10, petroleum ether/ethyl acetate) producing 52 mg (0.18 mmol, 50%) of -2-(2-methylnaphth-1-yl)-6-hydroxybenzo[β]thiophene as white semi crystals. ¹H NMR (CD₃COCD₃) 8.60 (s, 1H) 7.87-8.05 (m, 2H), 7.74 (d, J=8.7 Hz, 1H), 7.65-7.71 (m, 1H), 7.38-7.54 (m, 4H) 7.18 (s, 1H), 7.02 (dd, J=8.7, 2.2 Hz, 1H), 2.39 (s, 3H).

### EXAMPLE 11

2-(2,5-dimethyl-4-hydroxyphenyl)-6-hydroxybenzo[β]thiophene (7).

(a) The cross-coupling of 248 mg (1 mmol) 2,5-dimethyl-4-iodophenol, with 491 mg (1.5 mmol) of the product from 1(a) was accomplished by the procedure set forth in example 1(b). The crude product was purified on a chromatotron (silica, 9:1, petroleum ether/ethyl acetate) producing 130 mg (0.46 mmol, 46%) of 2-(2,5-dimethyl-4-hydroxyphenyl)-6-methoxybenzo[β]thiophene as white crystals. ¹H NMR (CDCl₃) 8.34 (s, 1H), 7.69 (d, J=8.8 Hz, 1H), 7.45 (d, J=2.3 Hz, 1H), 7.19 (s, 1H), 7.17 (s, 1H), 6.98 (dd, J=8.8, 2.3 Hz, 1H), 6.78 (s, 1H), 3.87 (s, 3H), 2.34 (s, 3H), 2.20 (s, 3H).

(b) The deprotection of 35 mg (0.12 mmol) of 2-(2,5-dimethyl-4-hydroxyphenyl)-6-methoxybenzo[β]thiophene (example (6a)) was accomplished by the procedure set forth in example 1(c). The crude product was purified on a chromatotron (silica, 70:30, petroleum ether/ethyl acetate) producing 26 mg (0.096 mmol, 80%) of 2-(2,5-dimethyl-4-hydroxyphenyl)-6-hydroxybenzo[β]thiophene as white crystals. MP 134-136° C., ¹H NMR (CD₃COCD₃) 8.41 (s broad, 2H) 7.63 (d, J =8.7 Hz, 1H), 7.30 (d, J=2.2 Hz, 1H), 7.18 (s, 1H), 7.13 (s, 1H) 6.93 (dd, J=8.7, 2.2 Hz, 1H), 6.77 (s,1H), 2.34 (s, 3H), 2.19 (s, 3H).

### EXAMPLE 12

2-(4-hydroxyphenyl)-6-hydroxybenzo[β]thiophene (8).

Prepared according to (Hauser et al, WO 96/30361).

### EXAMPLE 13

2-(2-benzylphenyl)-6-hydroxybenzo[β]thiophene (9).

The cross-coupling of 124 mg (0.5 mmol) 2-bromodiphenylmethane, with 246 mg (0.75 mmol) of the product from 1(a) was accomplished by the procedure set forth in example 1(b). The crude product was deprotected by the procedure set forth in example 1 (c). It was purified on a chromatotron (silica, 92:8, petroleum ether/ethyl acetate) producing 108 mg (0.34 mmol, 68%) 2-(2-benzylphenyl)-6-hydroxybenzo[β]thiophene as slightly pink crystals. ¹H NMR (CD₃COCD₃) 8.55 (s 1H) 7.62 (d, J=8.4 Hz, 1H), 7.04-7.51 (m, 11H), 6.93 (dd, J=8.5, 2.5 Hz, 1H), (s, 2H).

### EXAMPLE 14

2-(4-hydroxynapht-1-yl)-6-hydroxy[β]thiophene (10)

The cross-coupling of 119 mg (0.5 mmol) 1-bromo4-methoxynaphthalene, with 246 mg (0.75 mmol) of the product from 1(a) was accomplished by the procedure set forth in example 1(b). The crude product was deprotected by the procedure set forth in example 1 (c). It was purified on a chromatotron (silica, 8:2, petroleum ether/ethyl acetate) producing 9 mg (0.03 mmol, 6.2%) 2-(4-hydroxynapht-1-yl)-6-hydroxy[β]thiophene(10) as dark brown crystals. ¹H NMR (CD₃COCD₃) 9.34 (s, 1H), 8.55 (s 1H), 8.31-8.38 (m, 1H), 8.20-8.27 (m, 1H), 7.72 (d, J=8.4 Hz, 1H), 7.48-7.58 (m, 2H), 7.48 (d, J=7.7, 1H), 7.37 (d, J=2.2, 1H), 7.34 (s, 1H), 7.00 (d, J=7.7, 1H), 6.98 (dd, J=8.4, 2.2 Hz, 1H).

### EXAMPLE 15

2-(2-methyl-3-chlorophenyl)-6-hydroxybenzo[β]thiophene (11).

The cross-coupling of 126 mg (0.5 mmol) 2-chloro-6-iodotoluene, with 246 mg (0.75 mmol) of the product from 1(a) was accomplished by the procedure set forth in example 1(b). The crude product was deprotected by the procedure set forth in example 1 (c). It was then purified on a chromatotron (silica, 92:8, petroleum ether/ethyl acetate) producing 88 mg (0.32 mmol, 64.1%) of 2-(2-methyl-3-chlorophenyl)-6-hydroxybenzo[β]thiophene (11). ¹H NMR (CDCl₃) 7.72 (d, J=8.7 Hz, 1H), 7.29-7.41 (m, 2H), 7.27 (d, J=2.5, 1H), 7.17 (d, J=7.91, 1H), 7.10 (s, 1H), 6.92 (dd, J=8.7, 2.5 Hz, 1H), 2.46 (s, 3H).

### EXAMPLE 16

2-(2-methyl-5-chlorophenyl)-6-hydroxybenzo[β]thiophene (12).

The cross-coupling of 126 mg (0.5 mmol) 4-chloro-2-iodotoluene, with 246 mg (0.75 mmol) of the product from 1(a) was accomplished by the procedure set forth in example 1(b). The crude product was deprotected by the procedure set forth in example 1 (c). It was then purified on a chromatotron (silica, 92:8, petroleum ether/ethyl acetate) producing 88 mg (0.32 mmol, 64.1%) of 2-(2-methyl-5-chlorophenyl)-6-hydroxybenzo[β]thiophene (12). ¹H NMR (CDCl₃) 7.63 (d, J=8.7 Hz, 1H), 7.43 (d, J=2.0 Hz, 1H), 7.27 (d, J=2.2, 1H), 7.19-7.23 (m, 2H), 7.14 (s, 1H), 6.92 (dd, J=8.5, 2.2 Hz, 1H), 2.41 (s, 3H).

### EXAMPLE 17

2-(2-methyl-4-chlorophenyl)-6-hydroxybenzo[β]thiophene (13).

The cross-coupling of 103 mg (0.5 mmol) 2-bromo-5-chlorotoluene, with 246 mg (0.75 mmol) of the product from 1(a) was accomplished by the procedure set forth in example 1(b). The crude product was deprotected by the procedure set forth in example 1 (c). It was then purified on a chromatotron (silica, 92:8, petroleum ether/ethyl acetate) producing 118 mg (0.43 mmol, 85.9%) 2-(2-methyl-4-chlorophenyl)-6-hydroxybenzo[β]thiophene (13) as slightly pink crystals. ¹H NMR (CD₃COCD₃) 8.63 (s, 1H), 7.69 (d, J=8.7 Hz, 1H), 7.45 (d, J=8.4 Hz, 1H), 7.26-7.41(m, 4H), (dd, J=8.4, 2.2 Hz, 1H), 2.47 (s, 3H).

### EXAMPLE 18

2-(2,5-hydroxy4-bromophenyl)-6-hydroxybenzo[β]thiophene (14).

The cross-coupling of 222 mg (0.75 mmol) 1,4-dibromo-2,5-dimethoxybenzene with 367 mg (1.125 mmol) of the product from 1(a) was accomplished by the procedure set forth in example 1(b). It was then purified on a chromatotron (silica, 95:5, petroleum ether/acetone) producing 65.5 mg (0.17 mmol, 23%) of

2-(2,5-methoxy-4-bromophenyl)-6-methoxybenzo[β]thiophene. 25 mg (0.066 mmol) 14.1 mg (0.042 mmol, 63.4%) of 2-(2,5-hydroxy-4-bromophenyl)-6-hydroxybenzo[β]thiophene. ¹H NMR (CD₃COCD₃) 8.84 (s, 1H), 8.54 (s, 1H), 8.33 (d, J=8.74 Hz, 1H), 7.81 (s, 1H), 7.69 (d, J=8.4 Hz, 1H), 7.34 (d, J=2.2 Hz, 1H), 7.28 (s, 1H), 7.14 (s, 1H), 6.92 (dd, J=8.4, 2.2 Hz, 1H).

### EXAMPLE 19

2-(2-methyl-4-nitrophenyl)-6-hydroxybenzo[β]thiophene (15).

(a) The cross-coupling of 432 mg (2.0 mmol) 2-bromo-5- nitrotoluene with 982 mg (3.0 mmol) of the product from 1(a) was accomplished by the procedure set forth in example 1(b). The crude product was purified on a chromatotron (silica, 8:2, petroleum ether/ethyl acetate) producing 681 mg (about 75% pure) of 2-(2-methyl-4-nitrophenyl)-6-methoxybenzo[β]thiophene.

(b) The deprotection of 100 mg (0.33 mmol) 2-(2-methyl-4-nitrophenyl)-6-methoxybenzo[o]thiophene (example (15a)) was accomplished by the procedure set forth in example 1(c). The crude product was purified on a chromatotron (silica, 70:30, petroleum ether/ethyl acetate) producing 73 mg (0.26 mmol, 78%) of 2-(2-methyl-4-nitrophenyl)-6-hydroxybenzo[β]thiophene. ¹H NMR (CDCl₃) 8.16 (d broad, J=2.1 Hz, 1H) 8.08 (dd, J=8.5, 2.1 Hz, 1H), 7.68 (d, J=8.5 Hz, 1H), 7.60 (d, J=8.5, 1H), 7.29 (d, J=2.4 Hz, 1H) 7.27 (s, 1H), 6.94 (dd, J=8.5, 2.4 Hz, 1H), 5.15 (s, 1H), 2.59 (s, 3H).

### EXAMPLE 20

2-(2-methyl-4-aminophenyl)-6-hydroxybenzo[β]thiophene (16).

50 mg (0.18 mmol) of 2-(2-methyl-4-nitrophenyl)-6-hydroxybenzo[β]thiophene (example (15(b)) was dissolved in 5 ml of ethanol and 198 mg (0.88 mmol) of tin dichloride dihydrate was added. The mixture was heated to 70° C. under a nitrogen atmosphere for 3 hours. Hydrochloric acid (1 M) was added and then the aqueous phase was extracted with ethyl acetate. The combined organic phases were washed with brine, dried over magnesium sulphate and then concentrated in vacuo. The crude product was purified on a chromatotron (silica, 6:4, petroleum ether/ethyl acetate) producing 22 mg (0.086 mmol, 48%) of 2-(2-methyl-4-aminophenyl)-6-hydroxybenzo[β]thiophene. ¹H NMR (CD₃OD) 7.54 (d, J=8.6, 1H), 7.18 (d, J=2.4 Hz, 1H), 7.17 (d, J=8.6, 1 H), 7.00 (s, 1H), 6.84 (dd, J=8.6, 2,4 Hz, 1H), 6.64 (d, J=2.4 Hz, 1H), 6.58 (dd, J=8.6, 2.4 Hz), 2.37 (s, 3H).

### EXAMPLE 21

2-(2-methyl-3-nitrophenyl)-6-hydroxybenzo[β]thiophene (17).

(a) The cross-coupling of 432 mg (2.0 mmol) 2-bromo-6- nitrotoluene with 982 mg (3.0 mmol) of the product from 1(a) was accomplished by the procedure set forth in example 1(b). The crude product was purified on a chromatotron (silica, 95:5, petroleum ether/ethyl acetate) producing 114 mg (0.38 mmol, 13%) of 2-(2-methyl-3-nitrophenyl)-6-methoxybenzo[β]thiophene.

(b) The deprotection of 200 mg (0.67 mmol) 2-(2-methyl-3-nitrophenyl)-6-methoxybenzo[β]thiophene (example (17a)) was accomplished by the procedure set forth in example 1(c). The crude product was purified on a chromatotron (silica, 70:30, petroleum ether/ethyl acetate) producing 101 mg (0.35 mmol, 53%) of 2-(2-methyl-3-nitrophenyl)-6-hydroxybenzo[β]thiophene. ¹H NMR (CDCOCD₃) 8.67 (s, 1H), 7.86 (dd, J=8.0, 1.2 Hz, 1H), 7.76 (dd, J=7.7, 1.2 Hz, 1H), 7.73 (d, J=8.7 Hz, 1H), 7.54 (m, 1H), 7.38 (d, J=2.1 Hz, 1H), 7.36 (s, 1H), 7.00 (dd, J=8.7, 2,1 Hz, 1H), 5.15 (s, 1H), 2.52 (s, 3H).

### EXAMPLE 22

2-(2-methyl-3-aminophenyl)-6-hydroxybenzo[β]thiophene (18).

350 mg (1.23 mmol) of 2-(2-methyl-3-nitrophenyl)-6-hydroxybenzo[β]thiophene (example (17(b)) was dissolved in 10 ml of ethanol and 1384 mg (6.1 mmol) of tin dichloride dihydrate was added. The mixture was heated to 70° C. under a nitrogen atmosphere for 3 hours. Hydrochloric acid (1 M) was added and then the aqueous phase was extracted with ethyl acetate. The combined organic phases were washed with brine, dried over magnesium sulphate and then concentrated in vacuo. The crude product was purified on HPLC (reversed phase, C18, gradient acetonitrile/water+0.05 trifluoroacetic acid) producing 27 mg (0.10 mmol, 8.1%) of 2-(2-methyl-3-aminophenyl)-6-hydroxybenzo[β]thiophene. ¹H NMR (CD₃OD) 8.93 (s, 1H broad), 7.65 (d, J=8.8, 1H), 7.33 (d, J=2.5 Hz, 1H), 7.12 (s, 1H), 6.92-7.00 (m, 2H), 6.71-6.78 (m, 2H), 4.69 (s, 2H broad), 2.20 (s, 3H).

### EXAMPLE 23

2-(2-methyl-3-bromo-5-hydroxyphenyl)-6-hydroxybenzo[β]thiophene (19).

(a) The cross-coupling of 369 mg (1.3 mmol) 2,6-dibromo-4- methoxytoluene with 636 mg (1.95 mmol) of the product from 1(a) was accomplished by the procedure set forth in example 1(b). The crude product was purified on a chromatotron (silica. 98:2, petroleum ether/ethyl acetate) producing 220 mg (0.61 mmol, 46.6%) of 2-(2-methyl-3-bromo-5-methoxyphenyl)-6-methoxybenzo[β]thiophene.

(b) The deprotection of 70 mg (0.19 mmol) 2-(2-methyl-3-bromo-5-methoxyphenyl)-6-methoxybenzo[β]thiophene (example (19a)) was accomplished by the procedure set forth in example 1(c). The crude product was purified on a chromatotron (silica. 8:2, petroleum ether/ethyl acetate) producing 55 mg (0.16 mmol, 86%) of 2-(2-methyl-3-bromo-5- hydroxyphenyl)-6-hydroxybenzo[β]thiophene. ¹H NMR (CDCOCD₃) 8.66 (s, 1H), 7.69 (d, J=8.4, 1H), 7.34 (d, J=2.2 Hz, 1H), 7.23 (s, 1H), 7.15 (d, J=2.5 Hz, 1H), 6.99 (d, J=2.2 Hz, 1H), 7.00 (dd, J=8.4, 2,2 Hz, 1H), 6.94 (d, J=2.5 Hz, 1H), 2.38 (s, 3H).

### EXAMPLE 24

2-(2-methyl-5-hydroxyphenyl)-6-hydroxybenzo[β]thiophene (20).

a) 30 mg (0.08 mmol) of 2-(2-methyl-3-bromo-5-methoxyphenyl)-6-methoxybenzo[β]thiophene (example (19a) was dissolved in 2 ml of tetrahydrofuran. The mixture was cooled to −70° C. and butyllithium (0.12 mmol) was added to the reaction mixture. The reaction mixture was stirred for 2.5 hours at −70° C. and then at room temperature overnight. The reaction mixture was quenched with aqueous ammonium chloride, extracted with ethyl acetate and dried over magnesium sulphate. This produced 30 mg of crude 2-(2-methyl-5-methoxyphenyl)-6-methoxybenzo[β]thiophene.

b) The deprotection of 30 mg (0.10 mmol) 2-(2-methyl-5-methoxyphenyl)-6-methoxybenzo[β]thiophene (example (20a)) was accomplished by the procedure set forth in example 1(c). The crude product was purified on HPLC (reversed phase, C18, gradient, acetonitrile/water +0.05 trifluoroacetic acid) producing 6.7 mg (0.026 mmol, 24%) of 2-(2-methyl-5-hydroxyphenyl)-6-hydroxybenzo[β]thiophene. ¹H NMR (CD₃COCD₃) 8.55 (s, 1H), 8.29 (s, 1H), 7.67 (dd, J=8.5, 2.2 Hz, 1H), 7.33 (t, 1H), 7.24 (d, J=2.5, 1H), 7.13 (dd, J=8.2, 2.0 Hz, 1H), 6.92-6.99 (m, 2H), 6.76 (dt, 1H), 2.35 (d, J=2.2 Hz, 3H).

### EXAMPLE 25

2-phenyl-6-hydroxybenzo[β]thiophene (21).

The cross-coupling of 157 mg (1.0 mmol) bromobenzene with 491 mg (1.5 mmol) of the product from 1(a) was accomplished by the procedure set forth in example 1(b). The crude product was deprotected by the procedure set forth in example 1 (c). It was purified on a chromatotron (silica, 9:1, petroleum ether/ethyl acetate) producing 81 mg (0.36 mmol, 36%) 2-phenyl-6-hydroxybenzo[β]thiophene. ¹H NMR (CD₃COCD₃) 8.65 (s 1H), 7.60-7.75 (m, 4H), 7.28-7.48 (m, 4H), 6.95 (dd, J=8.5, 2.5 Hz, 1H).

### EXAMPLE 26

2-(4-hydroxyphenyl)-benzo[β]thiophene (22).

(a) The stannylation of 3 g (22.4 mmol) of benzo[β]thiophene was accomplished by the procedure set forth in example 1(a). This produced 6.3 g (21.3 mmol) of 2-trimethylstannylbenzo[β]thiophene.

(b) The cross-coupling of 468 mg (2.0 mmol) 4-iodophenol with 889 mg (3.0 mmol) of the product from 22(a) was accomplished by the procedure set forth in example 1(b). The crude product was deprotected by the procedure set forth in example 1 (c) and then purified on a chromatotron (silica, 9:1, petroleum ether/ethyl acetate) and recrystalised (petroleum ether/ethyl acetate) producing 20 mg (0.09 mmol, 4%) of 2-(4-hydroxyphenyl)-benzo[β]thiophene. ¹H NMR (CD₃COCD₃) 8.70 (s 1H), 7.88 (m, 1H), 7.79 (m, 1H), 7.56-7.69 (m, 3H), 7.25-7.38 (m, 2H), 6.90-7.00 (m, 2H).

### EXAMPLE 27

2-(2-trifluoromethyl-6-fluorophenyl)-6-hydroxybenzo[β]thiophene (23).

Produced in a parallell solution phase way. A mixture of 61 mg (0.25 mmol) 2-bromo-3-fluorobenzotrifluoride, 15 mg (0.013 mmol) tetrakis triphenylphosphinepalladium (0) and 20 mg (0.25 mmol) of cupric oxide in 1 ml of N,N-dimethylformamide was stirred at 100° C. under nitrogen. After 5 minutes, 123 mg (0.38 mmol) of 2-trimethylstannyl-6-methoxybenzo[β]thiophene (example 1(a)) in 2 ml of N,N-dimethylformamide was added all at once to the reaction mixture. The solution was heated to 100° C. for 3 hours, concentrated on a speed-vac. dissolved in dichloromethane, filtered through a silica pad and then concentrated again. The product was dissolved in 1.5 ml of dichloromethane and 1 ml of boron trifluoride dimethylsulfide complex was added. The reaction mixture was stirred overnight in darkness, quenched with water and extracted with dichloromethane. The organic phase was dried by passing it through sodium sulphate dryingtubes and then it was concentrated in a speed-vac. The crude product was purified on HPLC (silica, n-heptane+0.5% acetic acid to ethyl acetate+0.5% acetic acid as gradient eluent) producing 1.5 mg (0.005 mmol, 2%) of 2-(2-trifluoromethyl-6-fluorophenyl)-6-hydroxybenzo[β]thiophene. ¹H NMR (CD₃COCD₃) 8.72 (s, 1H broad), 7.71-7.78 (m, 3H), 7.55-7.65 (m, 1H), 7.37 (d, J=2.2. Hz, 1H), 7.29 (s, 1H), 6.99 (dd, J=8.7, 2.2 Hz, 1H).

### EXAMPLE 28

6-(6-hydroxy-2-benzo[β]thienyl)-4,5-dimethylbenzo-2,1,3-thiadiazole (24).

The cross-coupling of 61 mg (0.25 mmol) 6-bromo-4,5-dimethylbenzo-2, 1,3-thiadiazole with 123 mg (0.38 mmol) of the product from 1(a) and the subsequent deprotection was accomplished by the procedure set forth in example 23. The crude product was purified on HPLC (silica, n-heptane+0.5% acetic acid to ethyl acetate +0.5% acetic acid as gradient eluent) producing 3.6 mg (0.012 mmol, 4.6%) of 6-(6-hydroxybenzo[β]thien-2-yl)-4,5-dimethylbenzo-2,1,3-thiadiazole. ¹H NMR (CD₃COCD₃) 7.92 (s 1H), 7.74 (d, J=8.5, 1H), 7.38 (d, J=2.2 Hz, 1H), 7.37 (s, 1H), 7.01 (dd, J=8.5, 2.2 Hz, 1H), 2.76 (s, 3H), 2.50 (s, 3H).

### EXAMPLE 29

2-(4-methyl-3-thienyl)-6-hydroxybenzo[β]thiophene (25).

The cross-coupling of 44 mg (0.25 mmol) 3-bromo-4-methylthiophene with 123 mg (0.38 mmol) of the product from 1(a) and the subsequent deprotection was accomplished by the procedure set forth in example 23. The crude product was purified on HPLC (silica, n-heptane+0.5% acetic acid to ethyl acetate+0.5% acetic acid as gradient eluent) producing 22 mg (0.09 mmol, 36%) 2-(4-methyl-3-thienyl)-6-hydroxybenzo[β]thiophene. ¹H NMR (CD₃COCD₃) 8.57 (s 1H), 7.66 (d, J=8.4, 1H), 7.53 (d, J=3.2 Hz, 1H), 7.35 (s, 1H), 7.32 (m, 1H), 7.23 (m, 1H), 6.94 (dd, J=8.4, 2.2 Hz), 2.43 (d, J=0.74 Hz, 3H).

### EXAMPLE 30

2-(3,4,5-trimethyl-2-thienyl)-6-hydroxybenzo[β]thiophene (26).

The cross-coupling of 252 mg (01.0 mmol) 2-iodo-3,4,5-trimethylthiophene with 491 mg (1.5 mmol) of the product from 1(a) was accomplished by the procedure set forth in example 1(b). The crude 2-(3,4,5-trimethyl-2-thienyl)-6-methoxybenzo[β]thiophene was deprotected by the procedure set forth in example 1 (c) and then purified on a chromatotron (silica, 9:1, petroleum ether/ethyl acetate) producing 180 mg (0.625 mmol, 63%) of 2-(3,4,5-trimethyl-2-thienyl)-6-hydroxybenzo[β]thiophene. ¹H NMR (CD₃COCD₃) 8.55 (s, 1H), 7.67 (d, J=8.7 Hz, 1H), 7.32 (d, J=2.2 Hz, 1H), 7.06 (d, J=0.7 Hz, 1H), 6.95 (dd, J=8.7, 2.2 Hz, 1H), 2.34 (s, 3H), 2.30 (s 3H), 1.96 (s, 3H).

### EXAMPLE 31

2-(5-(1,3-dimethyluracilyl))-6-hydroxybenzo[β]thiophene (27).

(a) The cross-coupling of 266 mg (1.0 mmol) 5-iodo-1,3-dimethyluracil with 491 mg (1.5 mmol) of the product from 1(a) was accomplished by the procedure set forth in example 1(b). The crude product was purified on a chromatotron (silica, 40:1, dichloromethane/ethyl acetate) producing 211 mg (0.625 mmol, 63%) of 2-(5-(1,3-dimethyluracilyl))-6-methoxybenzo[β]thiophene.

(b) The deprotection of 30 mg (0.10 mmol) of 2-(5-(1,3-dimethyluracilyl))-6-methoxybenzo[β]thiophene (example (27a)) was accomplished by the procedure set forth in example 1(c). The crude product was purified on a chromatothron (silica, 9:1, petroleum ether/ethyl acetate) producing 1.2 mg (0.004 mmol, 4.2%) of 2-(5-(1,3-dimethyluracilyl))-6-hydroxybenzo[β]thiophene ¹H NMR (CDCl₃) 7.74 (s, 1H), 7.61 (d, J=8.7 Hz, 1H), 7.54 (s, 1H), 7.10-7.30 (m, 1H), 7.13 (dd. J=8.7, 2.3 Hz, 1H), 3.87 (s, 3H), 3.51 (s, 3H).

### EXAMPLE 32

[2-(4-hydroxyphenyl)-6-hydroxybenzo[β]thien-3-yl][phenyl]methanone (28).

(a) To 200 mg, (0.74 mmol) 6-methoxy-2-(4-methoxyphenyl)benzo[β]thiophene (Hauser et al, WO 96/30361) and 110 mg (0.78 mmol) benzoyl chloride in dichloro methane (5 ml) was added 740 mg (5.6 mmol) aluminium chloride. The reaction mixture was stirred for 5 hours at room temperature. The reaction was quenched by the addition of ethyl acetate and 1 M hydrochloric acid. The organic layer was separated and the aqueous phase was extracted with ethyl acetate. The combined organic phases were dried over magnesium sulphate, filtered and concentrated. The crude product was purified on a chromatotron (silica, 95:5, petroleum ether/ethyl acetate) producing 131 mg (0.35 mmol, 47%) of [2-(4-methoxyphenyl)-6-methoxybenzo[β]thien-3-yl]phenylmethanone as yellow crystals. ¹H NMR (CDCl₃) 7.72-7.80 (m, 2H) 7.59 (d, J=8.9 Hz, 1H), 7.36-7.43 (m, 1H), 7.21-7.34 (m, 5H), 6.97 (dd, J=8.9, 2.5 Hz, 2H) 6.72 (m, 1H), 3.88 (s, 3H), 3.72 (s, 3H).

(b) 70 mg, (0.19 mmol) [2-(4-methoxyphenyl)-6-methoxybenzo[β]thien-3-yl]phenylmethanone (example 28a) was dissolved in dichloromethane (5 ml), put under nitrogen atmosphere and cooled to −5° C. To the stirred solution was added 0.56 ml (0.56 mmol) 1M BBr₃ dropwise. The reaction mixture was stirred for 1 hour at 5° C., poured into ice water and extracted with ethyl acetate. The organic phase was dried over magnesium sulphate, filtered and concentrated. The crude product was purified on a chromatotron (silica, 75:25 to 50:50, petroleum ether/ethyl acetate as gradient eluent) producing 46 mg (0.13 mmol, 71%) of [2-(4-hydroxyphenyl)-6-hydroxybenzo[β]thien-3-yl]phenylmethanone as yellow crystals. MP 214-217° C., ¹H nmr (CD₃COCD₃) 8.74 (s, 1H), 8.64 (s, 1H), 7.70-7.77 (m, 2H), 7.20-7.54 (m, 7H), 6.96 (dd, J=8.7, 2.4 Hz, 1H), 6.68-6.76 (m, 2H).

### EXAMPLE 33

[2-(4-hydroxyphenyl)-6-hydroxybenzo[β]thien-3-yl][2-naphthyl]methanone (29).

(a) The acylation of 150 mg, (0.55 mmol) 6-methoxy-2-(4-methoxyphenyl)benzo[β]thiophene (Hauser et al, WO 96/30361) with 111 mg (0.58 mmol) 2-naphthoyl chloride was accomplished by the procedure set forth in example 28(a). The crude product was purified on a chromatotron (silica, 95:5, petroleum ether/ethyl acetate) producing 105 mg (0.25 mmol, 45%) of [2-(4-methoxyphenyl)-6-methoxybenzo[β]thien-3-yl][2-naphthyllmethanone as yellow crystals. ¹H nmr (CDCl₃) 8.21 (s, 1H), 7.95 (dd, J=8.7, 1.7 Hz, 1H), 7.71 (m, 3H), 7.31-7.61 (m, 6H), 6.96 (dd, J=8.9, 2.5 Hz, 1H), 6.65 (m, 2H), 3.88 (s, 3H), 3.63 (s, 3H).

(b) The deprotection of 70 mg (0.17 mmol) of [2-(4-methoxyphenyl)-6-methoxybenzo[β]thien-3-yl][2-naphthyl]methanone (example 29(a)) was accomplished by the procedure set forth in example 28 (b). The crude product was purified on a chromatotron (silica, 75:25 to 50:50, petroleum ether/ethyl acetate as gradient eluent) producing 47 mg (0. 12 mmol, 72%) [2-(4-hydroxyphenyl)-6-hydroxybenzo[β]thien-3-yl][2-naphthyl]methanone as yellow crystals. MP 229-232° C. ^{1H nmr (CD}₃COCD₃) 8.73 (s, 1H), 8.52 (s, 1H), 8.24 (s, 1H), 7.83-7.98 (m, 4H), 7.41-7.63 (m, 4H), 7.22-7.33 (m, 2H), 6.96 (dd, J=8.8, 2.2 Hz, 1H), 6.65 (m, 2H).

### EXAMPLE 34

[2-(4-hydroxyphenyl)-6-hydroxybenzo[β]thien-3-yl][4-tert-butylphenyl]methanone (30).

(a) The acylation of 150 mg, (0.55 mmol) 6-methoxy-2-(4-methoxyphenyl)benzo[β]thiophene (Hauser et al, WO 96/30361) with 115 mg (0.58 mmol) 4-tert-butylbenzoyl chloride was accomplished by the procedure set forth in example 28(a). The crude product was purified on a chromatotron (silica, 95:5, petroleum ether/ethyl acetate) producing 125 mg (0.29 mmol, 52%) of [2-(4-methoxyphenyl)-6-methoxybenzo[β]thien-3-yl][4-tert-butylphenyl]methanone as yellow crystals. ¹H nmr (CDCl₃) 7.64-7.73 (m, 2H), 7.55 (d, J=8.9 Hz, 1H), 7.22-7.34 (m, 5H), 6.95 (dd, J=8.9, 2.5 Hz, 1H), 6.72 (m, 2H), 3.87 (s, 3H), 3.71 (s, 3H), 1.22 (s, 9H).

(b) The deprotection of 70 mg (0.17 mmol) 2-(4-methoxyphenyl)-6-methoxybenzo[β]thien-3-yl][4-tert-butylphenyl]methanone (example (30a)) was accomplished by the procedure set forth in example 28 (b). The crude product was purified on a chromatotron (silica, gradient 75:25 to 50:50, petroleum ether/ethyl acetate) producing 30 mg (0.07 mmol, 46%) of [2-(4-hydroxyphenyl)-6-hydroxybenzo[β]thien-3-yl][4-tert-butylphenyl]methanone as yellow crystals. MP 197-200° C., ¹H nmr (CD₃COCDD₃) 8.69 (s, 1H), 8.60 (s, 1H), 7.63-7.70 (m, 2H), 7.35-7.46 (m, 4H), 7.21-7.28 (m, 2H), 6.94 (d, J=8.8, 2.2 Hz, 1H), 6.72 (m, 2H), 1.28 (s, 9H).

### EXAMPLE 35

[2-(4-hydroxyphenyl)-6-hydroxybenzo[β]thien-3-yl][4-methoxyphenyl]methanone (31).

(a) The acylation of 150 mg, (0.55 mmol) 6-methoxy-2-(4-methoxyphenyl)benzo[β]thiophene (Hauser et al, WO 96/30361) with 99 mg (0.58 mmol) 4-methoxybenzoyl chloride was accomplished by the procedure set forth in example 28(a). The crude product was purified on a chromatotron (silica, 95:5, petroleum ether/ethyl acetate) producing 112 mg (0.27 mmol, 50%) of [2-(4-methoxyphenyl)-6-methoxybenzo[β]thien-3-yl][4-methoxyphenyl]methanone as yellow crystals.

(b) The deprotection of 70 mg (0.17 mmol) 2-(4-methoxyphenyl)-6-methoxybenzo[β]thien-3-yl][4-methoxyphenyl]methanone(example (31a)) was accomplished by the procedure set forth in example 28 (b). The crude product was purified on a chromatotron (silica, 50:50, petroleum ether/ethyl acetate) producing 40 mg (0.07 mmol, 63%) of [2-(4-hydroxyphenyl)-6-hydroxybenzo[β]thien-3-yl][4-methoxyphenyl)methanone as yellow crystals. ¹H nmr (CD₃COCD₃) 8.71 (s, 2H broad), 7.73 (m, 2H), 7.38-7.42 (m, 2H), 7.28(m, 2H), 6.94 (dd, J=8.4, 2.4 Hz, 1H), 6.86 (m, 2H), 6.75 (m, 2H), 3.79 (s, 3H).

### EXAMPLE 36

[2-(4-hydroxyphenyl)-6-hydroxybenzo[β]thien-3-yl][4-carboxyphenyl]methanone (32).

(a) The acylation of 506 mg, (1.87 mmol) 6-methoxy-2-(4-methoxyphenyl)benzo[β]thiophene (Hauser et al, WO 96/30361) with 390 mg (1.97 mmol) terephthalic acid monomethyl ester chloride was accomplished by the procedure set forth in example 28(a). The crude product was purified on a chromatotron (silica, 8:2 petroleum ether/ethyl acetate) producing 442 mg (1.02 mmol, 55%) of [2-(2-(4-ethoxyphenyl)-6-methoxybenzo[β]thien-3-yl][4-methoxycarbonylphenyl]methanone.

(b) The deprotection of 406 mg (0.94 mmol) [2-(4-methoxyphenyl)-6-methoxybenzo[β]thien-3-yl][4-methoxycarbonylphenyl]methanone. (example (32a)) was accomplished by the procedure set forth in example 1 (c). The crude product was purified by recrystallisation (acetic acid/dichloromethane/methanol) producing 270 mg (0.69 mmol, 73%) of [2-(4-hydroxyphenyl)-6-hydroxybenzo[β]thien-3-yl][4-carboxyphenyl]methanone. ¹H nmr (CD₃COCD1) 8.76 (s, 1H broad), 8.63 (s, 1H broad), 7.92-7.98 (m, 2H), 7.76-7.83 (m, 2H), 7.63 (d, J=8.8 Hz, 1H), 7.41 (d, J 2.5 Hz, 1H), 7.21 (m, 2H), 6.99 (dd, J=8.8, 2.5 Hz, 1H), 6.69 (m, 2H).

### EXAMPLE 37

[2-(4-hydroxyphenyl)-6-hydroxybenzo[β]thien-3-yl][4-methoxycarbonylphenyl]methanone (33).

100 mg (0.25 mmol) [2-(4-hydroxyphenyl)-6-hydroxybenzo[β]thien-3-yl][4-carboxyphenyl]methanone (example 32(b)) were dissolved in methanol and five drops of thionyl chloride were added. The reaction mixture was stirred at room temperature for 24 hours, quenched with water, extracted with ethyl acetate and dried over magnesium sulphate. The crude product was purified on a chromatothron (silica, 9:1 petroleum ether/ethyl acetate) producing 42 mg (0.1 mmol, 42%) of 2-(4-hydroxyphenyl)-6-hydroxybenzo[β] thien-3-yl][4-methoxycarbonylphenyl]methanone. ¹H nmr (CD₃OD) 8.76 (s, 1H broad), 7.82-7.88 (m, 2H), 7.66-7.72 (m, 2H), 7.61 (d, J=8.9 Hz, 1H), 7.28 (d, J=2.4 Hz, 1H), 7.08-7.14 (m, 2H), 6.90 (dd, J=8.9, 2.4 Hz, 1H), 6.53-6.60 (m, 2H), 3.85 (s, 3H),

### EXAMPLE 38

[2-(4-hydroxyphenyl)-6-hydroxybenzo[β]thien-3-yl][4-ethoxycarbonylphenyl]methanone (34).

50 mg (0.12 mmol) [2-(4-hydroxyphenyl)-6-hydroxybenzo[β]thien-3-yl][4-carboxyphenyl]methanone (example 32(b)) were dissolved in ethanol and five drops of thionyl chloride were added. The reaction mixture was stirred at room temperature for 24 hours, quenched with water, extracted with ethyl acetate and dried over magnesium sulphate. The crude product was purified on a chromatothron (silica, 9:1 petroleum ether/ethyl acetate) producing 39 mg (0.1 mmol, 74%) of 2-(4hydroxyphenyl)-6-hydroxybenzo[β]thien-3-yl][4-ethoxycarbonylphenyl]methanone. ¹H nmr (CD₃OD) 8.72 (s, 2H broad), 7.87-7.94 (m, 2H), 7.76-7.82 (m, 2H), 7.62 (d, J=8.8 Hz, 1H), 7.42 (d, J=2.2 Hz, 1H), 7.17-7.24 (m, 2H), 6.99 (dd, J=8.8, 2.2 Hz. 1H), 6.66-6.72 (m, 2H), 4.31 (q, 2H), 1.32 (t, 3H).

### EXAMPLE 39

[2-(4-hydroxyphenyl)-6-hydroxybenzo[β]thien-3-yl][4-cyanophenyl]methanone (35).

(a) The acylation of 300 mg, (1.11 mmol) 6-methoxy-2-(4-methoxyphenyl)benzo[β]thiophene (Hauser et al, WO 96/30361) with 193 mg (1.17 mmol) 4-cyanobenzoyl chloride was accomplished by the procedure set forth in example 28(a). The crude product was purified on a chromatotron (silica, 8:2 petroleum ether/ethyl acetate) producing 206 mg (0.52 mmol, 46%) of [2-(4-methoxyphenyl)-6-methoxybenzo[β]thien-3-yl][4-cyanophenyl]methanone.

(b) The deprotection of 30 mg (0.075 mmol) [2-(4-methoxyphenyl)-6-methoxybenzo[β]thien-3-yl][4-cyanophenyl]methanone (example (35a)) was accomplished by the procedure set forth in example 1 (c). The crude product was purified on a chromatotron (silica, 5:5 petroleum ether/ethyl acetate) producing 24 mg (0.06 mmol, 86%) of [2-(4-hydroxyphenyl)-6-hydroxybenzo[β]thien-3-yl][4-cyanophenyl]methanone. ¹H nmr (CD₃COCD₃) 8.77 (s, 1H broad), 8.73 (s, 1H broad), 7.78-7.85 (m, 2H), 7.65-7.73 (m, 3H), 7.43 (d, J=2.2 Hz, 1H), 7.16 (m 2H), 7.02 (dd, J=8.8, 2.2 Hz, 1H), 6.69 (m, 2H).

### EXAMPLE 40

[2-(4-hydroxyphenyl)-6-hydroxybenzo[β]thien-3-yl][4-(1H-tetrazol-5-yl) phenyl]methanone (36).

30 mg (0.08 mmol) [2-(4-hydroxyphenyl)-6-hydroxybenzo[β]thien-3-yl][4-cyanophenyl]methanone (example 35(b)) was dissolved in 1 ml of N, N-dimethylformamide and kept under nitrogen. To the reaction mixture was added 49 mg (0.08 mmol) sodium azide and 40 mg (0.08 mmol) ammonium chloride, then it was heated to reflux temperature for 2 hours. The N, N-dimethylformamide was removed in a speed-vac. The compound was deprotected by the procedure set forth in example 1 (c). The crude product was purified on HPLC (reversed phase, C18, gradient acetonitrile/water+0.05 trifluoroacetic acid) producing 24 mg (0.06) mmol, 72%) of [2-(4-hydroxyphenyl)-6-hydroxybenzo[β]thien-3-yl][4-(1H-tetrazol-5-yl) phenyl]methanone. ¹H nmr (CD₃COCD₃) 8.80 (s, 1H), 8.65 (s, 1H), 8.00-8.12 (m, 2H), 7.86-7.92 (m, 2H), 7.62 (d, J=8.8 Hz, 1H), 7.42 (d, J=2.2 Hz, 1H), 7.18-7.28, (m, 2H), 7.00 (dd, J=8.8, 2.2 Hz, 1H), 6.65-6.75 (m, 2H).

### EXAMPLE 41

5-oxo-5-[2-(4-hydroxyphenyl)-6-hydroxybenzo[β]thien-3-yl]pentanoic acid methyl ester (37).

(a) The acylation of 200 mg, (0.74 mmol) 6-methoxy-2-(4-methoxyphenyl)benzo[β]thiophene (Hauser et al, WO 96/30361) with 139 mg (0.78 mmol) methyl adipoyl chloride was accomplished by the procedure set forth in example 28(a). The crude product was purified on a chromatotron (silica, 9:1 petroleum ether/ethyl acetate) producing 91 mg (0.22 mmol, 30%) of 5-oxo-5-[2-(4-methoxyphenyl)-6-methoxybenzo[β]thien-3-yl]pentanoic acid methyl ester.

(b) The deprotection of 80 mg (0.19 mmol) 5-oxo-5-[2-(4-methoxyphenyl)-6-methoxybenzo[β]thien-3-yl]pentanoic acid methyl ester (example (37a)) was accomplished by the procedure set forth in example 1 (c). The crude product was purified on a chromatotron (silica, 98:2 chloroform/methanol+acetic acid) producing 38 mg (0.10 mmol, 52%) of 5-oxo-5-[2-(4-hydroxyphenyl)-6-hydroxybenzo[β]thien-3-yl]pentanoic acid methyl ester. ¹H nmr (CD₃COCD₃) 8.86 (s, 1H broad), 8.67 (s, 1H broad), 7.82 (d, J=8.8 Hz, 1H), 7.28-7.40 (m, 3H), 6.95-7.05 (m 3H), 3.56 (s, 3H), 2.37-2.46 (m, 2H), 2.11-2.20 (m, 2H), 1.36-1.60 (m, 4H).

### EXAMPLE 42

5-oxo-5-[2-(4-hydroxyphenyl)-6-hydroxybenzo[β]thien-3-yl]pentanoic acid (38).

25 mg (0.06 mmol) of 5-oxo-5-[2-(4-hydroxyphenyl)-6-hydroxybenzo[β]thien-3-yl]pentanoic acid methyl ester (example 37(b) was dissolved in five ml of methanol and 0.5 ml of 1 M sodium hydroxide. The reaction mixture was stirred for one hour, neutralized, extracted with ethyl acetate and dried over magnesium sulphate. This produced 11 mg (0.03 mmol, 49%) of 5-oxo-5-[2-(4-hydroxyphenyl)-6-hydroxybenzo[β]thien-3-yl]pentanoic acid. ¹H nmr (CD₃COCD₃) 7.82 (d, J=8.8 Hz, 1H), 7.28-7.40 (m, 3H), 6.90-7.05 (m 3H).

### EXAMPLE 43

[2-(4-hydroxyphenyl)-6-hydroxybenzo[β]thien-3-yl][4-propylpheny]methanone (39).

(a) The acylation of 200 mg, (0.74 mmol) 6-methoxy-2-(4-methoxyphenyl)benzo[β]thiophene (Hauser et al, WO 96/30361) with 142 mg (1.17 mmol) 4-propylbenzoyl chloride was accomplished by the procedure set forth in example 28(a). The crude product was purified on a chromatotron (silica, 9:1 petroleum ether/ethyl acetate) producing 128 mg (0.52 mmol, 42%) of [2-(4-methoxyphenyl)-6-methoxybenzo[β]thien-3-yl][4-isopropylphenyl]methanone.

(b) The deprotection of 100 mg (0.24 mmol) [2-(4-methoxyphenyl)-6-methoxybenzo [β]thien-3-yl][4-isopropylphenyl]methanone. (example (39a)) was accomplished by the procedure set forth in example 1 (c). The crude product was purified on a chromatotron (silica, 5:5 petroleum ether/ethyl acetate) producing 28 mg (0.07 mmol, 30%) of [2-(4-hydroxyphenyl)-6-hydroxybenzo [β]thien-3-yl][4-propylphenyl]methanone. ¹H nmr (CD₃COCD₃) 8.71 (s, 1H), 8.62 (s, 1H), 7.62-7.70 (m, 2H), 7.45 (d, J=8.8 Hz, 1H), 7.39 (d, J=2.2 Hz, 1H), 7.21-7.28 (m 2H), 7.14-7.20 (m, 2H), 6.94, (dd, J=8.8, 2.2 Hz, 1H), 6.68-6.75 (m, 2H), 2.55 (t, 2H), 1.58 (m, 2H), 0.88 (t, 3H).

### EXAMPLE 44

[2-(4-hydroxyphenyl)-6-hydroxybenzo[β]thien-3-yl][4-iodophenyl]methanone (40).

a) The acylation of 200 mg, (0.74 mmol) 6-methoxy-2-(4-methoxyphenyl)benzo[β]thiophene (Hauser et al, WO 96/30361) with 207 mg (0.77 mmol) 4-iodobenzoyl chloride was accomplished by the procedure set forth in example 28(a). The crude product was purified on a chromatotron (silica, 9:1 petroleum ether/ethyl acetate) producing 258 mg (0.52 mmol, 70%) of [2-(4-methoxyphenyl)-6-methoxybenzo[β]thien-3-yl][4-iodophenyl]methanone.

(b) The deprotection of 100 mg (0.20 mmol) [2-(4-methoxyphenyl)-6-methoxybenzo [β]thien-3-yl][4-iodophenyl]methanone. (example (40)) was accomplished by the procedure set forth in example 1 (c). The crude product was purified on a chromatotron (silica, 5:5 petroleum ether/ethyl acetate) producing 43 mg (0.09 mmol, 45%) of [2-(4-hydroxyphenyl)-6-hydroxybenzo[β]thien-3-yl][4-iodophenyl]methanone. ¹H nmr (CD₃COCD₃) 8.71 (s, 2H, broad), 8.62 (s, 1H), 7.69-7.78 (m, 2H), 7.54 (d, J=8.8 Hz, 1H), 7.45-7.50 (m, 2H), 7.40 (d, J=2.2 Hz, 1H), 7.17-7.24 (m 2H), 6.96, (dd, J=8.8, 2.2 Hz, 1H), 6.68-6.75 (m, 2H).

### EXAMPLE 45

2-(4-hydroxyphenyl)-6-hydroxybenzo[β]thien-3-yl][4-acetylphenyl]methanone (41).

(a) A mixture of 246 mg (0.75 mmol) of hexamethylditin, 250 mg (0.50 mmol) of [2-(4-methoxyphenyl)-6-methoxybenzo[β]thien-3-yl][4-iodophenyl] methanone. (example (39a)), 6 mg (0.005 mmol) ) tetrakis triphenylphosphinepalladium (0) and 20 ml toluene was heated under reflux in a nitrogen atmosphere for 20 h. The reaction mixture was concentrated, dissolved in diethylether, washed with water twice, dried over magnesium sulphate, filtered and concentrated. This yielded 241 mg (0.45 mmol, 90%) of the desired [2-(4-methoxyphenyl)-6-methoxybenzo[β]thien-3-yl][4-trimethylstannylphenyl]methanone.

(b) 100 mg (0.19 mmol) of [2-(4-methoxyphenyl)-6-methoxybenzo[β]thien-3-yl][4-trimethylstannylphenyl]methanone (example 41(a)) and 15 mg (0.19 mmol) of acetyl chloride was dissolved in 5 ml of toluene. To the reaction mixture was added 4.6 mg (0.0044 mmol) of tris(dibenzylideneacetone)palladium(0)*chloroform. The reaction mixture was then heated under a nitrogen atmosphere at 70\mathring{5} C 20 hours, filtered, extracted with ethyl acetate, washed with saturated sodium bicarbonate and dried over magnesium sulphate. The deprotection of the crude [2-(4-methoxyphenyl)-6-methoxybenzo[βthien-3-yl][4-acetylphenyl]methanone was accomplished by the procedure set forth in example 1 (c). The product was purified on a chromatotron (silica, 5:5 petroleum ether/ethyl acetate) producing 61 mg (0.16 mmol, 82%) of [2-(4-hydroxyphenyl)-6-hydroxybenzo[β]thien-3-yl][4-acetylphenyl]methanone. ¹H nmr (CD₃COCD₃) 7.85-7.92 (m, 2H), 7.76-7.83 (m, 2H), 7.59 (d, J=8.8 Hz, 1H), 7.41 (d. J=2.2 Hz, 1H), 7.17-7.24 (m 2H), 6.98, (dd, J=8.8, 2.2 Hz, 1H), 6.66-6.73 (m, 2H), 2.54 (s, 3H).

### EXAMPLE 46

2-(4-hydroxyphenyl)-6-hydroxybenzo[β]thien-3-yl][4-propionylphenyl]methanone (42).

200 mg (0.38 mmol) of [2-(4-methoxyphenyl)-6-methoxybenzo[β]thien-3-yl][4-trimethylstannylphenyl]methanone (example 41(a)) and 34 mg (0.38 mmol) of propionyl chloride was dissolved in 10 ml of toluene. To the reaction mixture was added 9.2 mg (0.0088 mmol) of tris(dibenzylideneacetone)palladium(0)*chloroform. The reaction mixture was then heated under a nitrogen atmosphere at 70° C. for 20 hours, filtered, extracted with ethyl acetate, washed with saturated sodium bicarbonate and dried over magnesium sulphate. The deprotection of the crude [2-(4-methoxyphenyl)-6-methoxybenzo[β]thien-3-yl][4-propionylphenyl]methanone was accomplished by the procedure set forth in example 1 (c). The crude product was purified on HPLC (reversed phase, C18, gradient acetonitrile/water+0.05 trifluoroacetic acid) producing 9 mg (0.02 mmol, 6%) of [2-(4-hydroxyphenyl)-6-hydroxybenzo[β]thien-3-yl][4-propionylphenyl]methanone. ¹H nmr (CD₃COCD₃) 8.72 (s, 1H), 8.61 (s, 1H), 7.85-7.92 (m, 2H), 7.76-7.83 (m, 2H), 7.59 (d, J=8.8 Hz, 1H), 7.41 (d, J=2.2 Hz, 1H), 7.17-7.24 (m 2H), 6.98, (dd, J=8.8, 2.2 Hz, 1H), 6.6-6.73 (m, 2H), 2.99 (q, 2H), 1.08 (t. 3H).

### EXAMPLE 47

2-(4-hydroxyphenyl)-6-hydroxybenzo[β]thien-3-yl][4-buturylphenyl]methanone (43).

100 mg (0.19 mmol) of [2-(4-methoxyphenyl)-6-methoxybenzo[β]thien-3-yl][4-trimethylstannylphenyl]methanone (example 41(a)) and 22 mg (0.20 mmol) of buturyl chloride was dissolved in 5 ml of toluene. To the reaction mixture was added 4.6 mg (0.0044 mmol) of tris(dibenzylideneacetone)palladium(0)*chloroform. The reaction mixture was then heated under a nitrogen atmosphere at 70° C. for 20 hours, filtered, extracted with ethyl acetate, washed with saturated sodium bicarbonate and dried over magnesium sulphate. The crude product was purified on a chromatotron (silica, 9:1 petroleum ether/ethyl acetate). The deprotection of the crude [2-(4-methoxyphenyl)-6-methoxybenzo[β]thien-3-yl][4-buturylphenyl]methanone was accomplished by the procedure set forth in example 1 (c). The product was purified on a chromatotron (silica, 9:1 petroleum ether/ethyl acetate) producing 17 mg (0.04 mmol, 22%) of [2-(4-hydroxyphenyl)-6-hydroxybenzo[β]thien-3-yl][4-buturylphenyl]methanone. ¹H nmr (CD₃COCD₃) 8.72 (s, 1H), 8.61 (s, 1H), 7.85-7.92 (m, 2H), 7.76-7.83 (m, 2H), 7.60 (d, J=8.8 Hz, 1H), 7.42 (d, J=2.2 Hz, 1H), 7.17-7.24 (m 2H), 6.99, (dd, J=8.8. 2.2 Hz, 1H), 6.66-6.73 (m, 2H), 2.96 (q, 2H), 1.67 (m, 2H), 1.08 (t. 3H).

### EXAMPLE 48

[2-(4-hydroxyphenyl)-6-hydroxybenzo[β]thien-3-yl][4-ethylthiocarbonylphenyl]-methanone (44).

50 mg (0.12 mmol) [2-(4-hydroxyphenyl)-6-hydroxybenzo[β]thien-3-yl][4-carboxyphenyl]methanone (example 32(b)) were dissolved in ethanthiol and five drops of thionyl chloride were added. The reaction mixture was stirred at room temperature for 24 hours in a nitrogen atmosphere, quenched with water, extracted with ethyl acetate and dried over magnesium sulphate. The crude product was purified on HPLC (reversed phase, C18, gradient acetonitrile/water+0.05 trifluoroacetic acid) producing 26 mg (0.06 mmol, 54%) of 2-(4-hydroxyphenyl)-6-hydroxybenzo[β]thien-3-yl][4-et-hylthiocarbonylphenyl]methanone. ¹H nmr (CD₃OD) 8.72 (s, 1H broad), 8.60 (s, 1H), 7.89-7.98 (m, 2H), 7.75-7.84 (m, 2H), 7.61 (d, J=8.8 Hz, 1H), 7.42 (d, J=2.5 Hz, 1H), 7.17-7.24 (m, 2H), 6.99 (dd, J=8.8, 2.5 Hz, 1H), 6.66-6.72 (m, 2H), 2.89 (q, 2H), 1.32 (t, 3H).

### EXAMPLE 49

2-(4-hydroxyphenyl)-6-hydroxybenzo[β]thien-3-yl][4-hydroxyphenyl]methanone (45)

The deprotection of 2-(4-methoxyphenyl)-6-methoxybenzo[β]thien-3-yl][4-methoxyphenyl]methanone (example (31a)) as described in example 31(b) produced after purification 2-(4-hydroxyphenyl)-6-hydroxybenzo[β]thien-3-yl][4-hydroxyphenyl]methanone as a byproduct. ¹H nmr (CD₃COCD₃) 8.60-9.00 (s, 3H broad), 7.62-7.72 (m, 2H), 7.36-7.40 (m, 2H), 7.22-7.28 (m, 2H), 6.92 (dd, J=8.6, 2.4 Hz, 1H), 6.68-6.80 (m, 4H).

### EXAMPLE 50

2-(4-hydroxyphenyl)-6-hydroxybenzo[β]thien-3-yl][4-methylamino carbonyl-phenyl]methanone (46)

2-(4-methoxyphenyl)-6-methoxybenzo[β]thien-3-yl][4-methoxycarbonylphenyl]methanone (example 32(b)) was deprotected to 2-(4-methoxyphenyl)-6-methoxybenzo[β] thien-3-yl][4-carboxyphenyl]methanone by dissolving in ethanol and stirring with 1M sodium hydroxide for 4 hours. The ethanol was evaporated and the aqueous phase was extracted with ethylacetate, dried over magnesium sulphate and evaporated. The following reaction was run in a parallell solution phase way. 20 mg (0.048 mmol) of 2-(4-methoxyphenyl)-6-methoxybenzo[β]thien-3-yl][4-carboxyphenyl]methanone was mixed in sequential order with 30 mg (0.058 mmol) of benzotriazole-1-yl-oxy-tris-pyrrolidino-phosphonium hexafluorophosphate (PyBOP), 3.6 mg (0.024 mmol) of N-hydroxybenzotriazole*H₂O (HOBt), 2.5 ml of N,N-dimethylformylamide, 12.4 mg (0.096 mmol) of N,N-diisopropylethylamine and 2.23 mg (0.096 mmol) of methylamine hydrochloride in a nitrogen atmosphere at room-temperature for 3 days. The reaction was diluted with ethyl acetate. The organic phase was washed with 10% citric acid, dried by passing through a 3 ml extube. Varian Chem. Elut. and concentrated on a speed-vac. The deprotection of 2-(4-methoxyphenyl)-6-methoxybenzo[β]thien-3-yl][4-methylaminocarbonylphenyl]methanone as described in example 1(c) produced after purification on HPLC (reversed phase, C18, gradient acetonitrile/water+0.05 trifluoroacetic acid) 6 mg (0.015 mmol, 31%) of 2-(4-hydroxyphenyl)-6-hydroxybenzo[β]thien-3-yl][4-methylaminophenyl]methanone. ¹H nmr (CD₃COCD₃) 7.70-7.85 (m, 4H), 7.55 (d, J=8.8, 1H), 7.41 (d, J=2.2, 1H), 7.19-7.25 (m, 2H), 6.99 (d, J=8.8, 2.2 Hz, 1H), 6.66-6.74 (m, 2H).

### EXAMPLE 51

2-(4-hydroxyphenyl)-6-hydroxybenzo[β] thien-3-yl][4-isobutylaminocarbonylphenyl]methanone (47)

This reaction was run in a parallell solution phase way by the procedure set forth in example 46, using isobutylamine hydrochloride (5.24 mg (0.096 mmmol)) instead of methylamine. The crude product was purified on HPLC (reversed phase, C18, gradient acetonitrile/water+0.05 trifluoroacetic acid) producing 1.2 mg (0.0027 mmol, 5.6%) of 2-(4-hydroxyphenyl)-6-hydroxybenzo[β]thien-3-yl][4-isobutylaminophenyl]-methanone. ¹H nmr (CD₃COCD₃) 7.72-7.92 (m, 4H), 7.51 (d, J=8.8, 1H), 7.42 (d, J=2.2 Hz, 1H), 7.18-7.26 (m, 2H), 6.97 (dd, J=8.8, 2.2 Hz, 1H), 6.66-6.76 (m, 2H).

### EXAMPLE 52

2-(4-hydroxyphenyl)-6-hydroxybenzo[β] thien-3-yl]-[4-benzylaminocarbonylphenyl]methanone (48)

This reaction was run in a parallell solution phase way by the procedure set forth in example 46, using benzylamine hydrochloride (7.68 mg (0.096 mmol) instead of methylamine. The crude product was purified on HPLC (reversed phase, C18, gradient acetonitrile/water+0.05 trifluoroacetic acid) producing 1.7 mg (0.0035 mmol, 7.4%) of 2-(4-hydroxyphenyl)-6-hydroxybenzo[β]thien-3-yl][4-bensylaminophenyl]-methanone. ¹H nmr (CD₃COCD₃) 8.33 (s, broad), 7.85-7.91 (m, 2H). 7.75-7.82 (m, 2H), 7.53 (d, J=8.8 Hz, 1H), 7.42 (d, J=2.2 Hz, 1H), 7.18-7.36 (m, 7H), 6.98 (dd, J=8.8, 2.2 Hz, 1H), 6.68-6.74 (m, 2H).

### EXAMPLE 53

15 mg (0.040 mmol) of 2-(4-hydroxyphenyl)-6-hydroxybenzo[β]thien-3-yl][4-carboxyphenyl]methanone (example 32(b)) was mixed in sequential order with 40 mg (0.077 mmol) of benzotriazole-1-yl-oxy-tris-pyrrolidino-phosphonium hexafluorophosphate (PyBOP), 4.9 mg (0.032 mmol) of N-hydroxybenzotriazole*H₂O (HOBt), 3.0 ml of N)N-dimethylformylamide, 16.5 mg (0.128 mmol) of N,N-diisopropyl-ethylamine and 0.015 g (0.096 mmol) of L-serine methylester hydrochloride in a nitrogen atmosphere at room-temperature for 3 days. The reaction was diluted with ethyl acetate. The organic phase was washed with 1M hydrochloric acid and brine. Then dried over magnesium sulphate and evaporated to dryness. Purification on HPLC (reversed phase, C18, gradient acetonitrile/water+0.05 trifluoroacetic acid) produced 7.4 mg (0.015 mmol, 38%) of 49. ¹H nmr (CD₃COCD₃) 7.78-7.92 (m, 4H), 7.55 (d, J=8.8, 1H), 7.41 (d, J=2.2, 1H), 7.19-7.28 (m, 2H), 6.98 (dd, J=8.8, 2.2 Hz, 1H), 6.66-6.74 (m, 2H), 4.69 (m, 1H), 3.95 (m, 2H), 3.69 (s, 3H).

### EXAMPLE 54

15 mg (0.040 mmol) of 2-(4-hydroxyphenyl)-6-hydroxybenzo[β] thien-3-yl][4-carboxyphenyl]methanone (example 32(b)) was mixed in sequential order with 40 mg (0.077 mmol) of benzotriazole-1-yl-oxy-tris-pyrrolidino-phosphonium hexafluoro-phosphate (PyBOP), 4.9 mg (0.032 mmol) of N-hydroxybenzotriazole*H₂O (HOBt), 3.0 ml of N,N-dimethylformylamide, 16.5 mg (0.128 mmol) of N,N-diisopropyl-ethylamine and 0.013 g (0.096 mmol) of L-alanine methylester hydrochloride in a nitrogen atmosphere at room-temperature for 3 days. The reaction was diluted with ethyl acetate. The organic phase was washed with 1M hydrochloric acid and brine. Then dried over magnesium sulphate and evaporated to dryness. Purification on HPLC (reversed phase, C18, gradient acetonitrile/water+0.05 trifluoroacetic acid) produced 17.4 mg (0.037 mmol, 91%) of 50. ¹H nmr (CD₃COCD₃) 8.12 (s, 1H broad), 8.10 (s, 1H broad), 7.75-7.89 (m, 4H), 7.53 (d, J=8.8, 1H), 7.41 (d, J=2.2, 1H), 7.17-7.23 (m, 2H), 6.98 (dd, J=8.8, 2.2 Hz, 1H), 6.69-6.74 (m, 2H), 4.59 (m, 1H), 3.66 (s, 3H), 1.45 (d, 3H).

### EXAMPLE 55

15 mg (0.040 mmol) of 2-(4-hydroxyphenyl)-6-hydroxybenzo[β]thien-3-yl][4-carboxyphenyl]methanone (example 32(b)) was mixed in sequential order with 40 mg (0.077 mmol) of benzotriazole-1-yl-oxy-tris-pyrrolidino-phosphonium hexafluoro-phosphate (PyBOP), 4.9 mg (0.032 mmol) of N-hydroxybenzotriazole*H₂O (HOBt), 3.0 ml of N,N-dimethylformylamide, 16.5 mg (0.128 mmol) of N,N-diisopropyl-ethylamine and 0.021 g (0.096 mmol) of L-phenylalanine methylester hydrochloride in a nitrogen atmosphere at room-temperature for 3 days. The reaction was diluted with ethyl acetate. The organic phase was washed with 1M hydrochloric acid and brine. Then dried over magnesium sulphate and evaporated to dryness. Purification on HPLC (reversed phase, C18, gradient acetonitrile/water+0.05 trifluoroacetic acid) produced 10.9 mg (0.020 mmol, 51%) of 51. ¹H nmr (CD)₃COCD₃) 8.01 (s, 1H broad), 7.98 (s, 1H broad), 7.75 (s, 4H), 7.53 (d, J=8.8, 1H), 7.41 (d, J=2.2, 1H), 7.15-7.31 (m, 7H), 6.98 (dd, J=8.8, 2.2 Hz, 1H), 6.69-6.74 (m, 2H), 4.82 (m, 1H), 3.65 (s, 3H).

The biological character of the compounds prepared in accordance with Examples 1 to 26 and 28 to 40 inclusive and also, for comparison purposes estradiol was measured in a radioligand displacement assay. The affinity for ERα and ERβ was measured as an IC₅₀, the concentration of ligand necessary to displace 50% of tritated 17-β-estradiol from either hERα (human estrogen receptor α) or hERβ (human estrogen receptor β) respectively. In this assay, it was found that the IC₅₀'s of compounds varied from 2.0 nM to 20 μM for ERα and from 2.0 nM to 12 μM for ERβ. The ERαERβ selectivity ratio varied from 0.2 to 23.

Experimental Description of ER Binding Assay

Affinity for the ER (by displacement of ³[H]-estradiol) ws measured using the scintistrip assay¹. Human estrogen receptors (hER) alpha and beta were extracted from the nuclei from SF9-cells infected with a recombinant baculovirus transfer vector containing the cloned hER genes.² The concentration of hER's in the extract was measured as specific ³[H]-E2 binding with the G25-assay.³

1) Haggblad, J., Carlsson, B., Kivelä, P., Siitari, H., (1995) Biotechniques 18, 146-151.

2) Barkhem, T., Carlsson, B., Simons, J., Moller, B., Berkenstam, A., Gustafsson J. A. G., Nilsson, S. (1991) J. Steroid Biochem. Molec. Biol. 38, 667-75.

3) Salononsson, M., Carlsson, B., Haggblad, J., (1994) J. Steroid Biochem. Molec. Biol. 50, 313-318.

1. A crystal comprising at least a portion of the ERα ligand binding domain.

2. A crystal according to claim 1 comprising at least 200 amino acids of ERα.

3. A crystal according to claim 1 or claim 2 comprising at least 250 amino acids of ERα.

4. A crystal according to claim 1, 2 or 3 comprising entire ERα.

5. A crystal according to any preceding claim produced using a sequence including helix H₁₂ of ERα.

6. A crystal according to any one of claims 1 to 5 usable in X-ray crystallography techniques.

7. A crystal according to any one of claims 1 to 6 including a ligand bound to ERα or a portion thereof.

8. A crystal according to claim 7 in which the ligand is estradiol, raloxifene, or any other ligand that binds with high affinity (<10 μM) to ERα.

9. A crystal of ERα LBD according to any preceding claim belonging to the space croup P2 and having the unit cell dimensions a=61.48 Å, b=115.16 Å. c=137.38 Å.

10. A crystal of ERα LBD according to any preceding claim belonging to the space group P2 and having the unit cell dimensions a=104.53 Å b=53.68 Å c=102.71 Å and β=116.79°.

11. A crystal of ERα LBD according to any one of claims 1 to 9 belonging to the space group C2 and having the unit cell dimensions a=89.91 Å b=75.09 Å c=87.50 Å and β=103.01°.

12. A crystal of ERα LBD according to any one of claims 1 to 9 belonging to the space group C222₁ and having the unit cell dimensions a=65.47 Å b=95.99 Å c=164.14 Å.

13. A method for designing ligands which will bind to an estrogen receptor, the method comprising determining amino acid or acids of the ligand binding domain of the estrogen receptor which interact with a binding ligand, and selecting a ligand which is likely to bind to the receptor according to the structure of the potential ligand.

14. A method according to claim 13 in which interaction with ERα and ERβ are separately determined whereby ER-form selective ligands can be selected.

15. A method according to claim 13 or 14, in which for ERα selective ligands the design of the potential ligand uses a crystal according to any one of claims 1 to 12.

16. Ligands for estrogen receptors designed using a method according to claim 13, 14 or 15.

17. Ligands designed according to a method according to claim 14 which are specific for ERα or ERβ.

18. Ligands binding to at least the LBD of an ER with an affinity of between 20 pmol and 200 nM.

19. Ligands binding reversibly to at least the LBD of an ER.

20. A method of inhibiting estadiol activity in an animal, the method comprising administering to the animal a ligand according to claim 19 or claim 20.

21. A method of inhibiting estradiol activity according to claim 20 comprising administering a ligand according to claim 18 or claim 19.

22. A pharmaceutical compound comprising a ligand according to any one of claims 16 to 19.

23. An estrogen agonist, an estrogen antagonist, a partial estrogen agonist, or a partial estrogen antagonist designed using a method according to claim 13, 14 or 15.

24. An ERα selective ligand having a structural group larger than methyl capable of fitting into the β cavity of the ERα.

25. An ERα selective ligand having the general formula Z 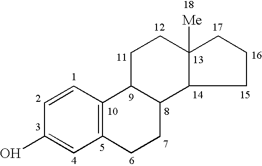
and having hydrophobic substituents at one or more of the 8β, 15β or 18 positions.

26. An ERβ selective ligand having the formula Z of claim 25 and having hydrophobic substituents at one or more of the 9α or 12α positions.

27. An ERα selective ligand according to claim 25 or ERβ selective ligand according to claim 26 in which the hydrophobic substituent is selected from methyl groups, ethyl groups, iso-propyl groups, chlorine, bromine or iodine.

28. An ERα or ERβ selective ligand, in which the ligand is a 2′-, 3′-, 5′- and/or 6′-substituted 2-aryl benzothiophene.

29. An ERα or ERβ selective ligand according to claim 28, which is substituted at one or more of the 2′, 3′, 5′ and 6′ positions.

30. An ERα selective ligand according to claim 28, in which the substituted 2-aryl benzothiophene fills the α- and β-face cavities of the ER.

31. An ERα selective ligand, which is a 2-aryl benzotheiphene with a small hydrophobic substituent at one or more of the 2′, 3′, 5′ and 6′ positions.

32. An ER ligand capable of filling the hydrophobic cavity of ER-α.

33. A ligand according to claim 32 which has a hydrophobic substituent on the ethoxyphenyl sidechain to the piperidinyl nitrogen atom of raloxifene.

34. A ligand according to claim 31 or 32 in which the ligand has a hydrophobic sustituent selected from linear alkyl groups, perfluoroalkyl groups (—CH₃ to —CH₁₀H₂₁, —CF₃ to —C₁₀F₂₁), benzyl-(CH₂Ph), benzyl-(methylene cyclohexyl groups).

35. An ER ligand having a structure capable of interacting with Glu-353 of ERα or with Glu-262 of ERβ.

36. An ER ligand having a structure capable of interacting with Arg-394 of ERα or with Arg-303 or ERβ.

37. An ER ligand having a structure capable of interacting with residue His-524 of ERα or with His-432 of ERβ.

38. An ER ligand having a structure capable of interacting with Met-421 or Leu-384 of ERα or with Ile-330 Met-293 of ERβ.

39. An ERα selective ligand having a structure capable of interacting with Met-421 and/or Leu-384 of ERα.

40. An ERβ selective ligand having a structure capable of interaction with Ile-330 and/or Met-293 of ERβ.

41. ERβ selective ligand according to claim 40 in which substitutions larger than a methyl group are provided at the α 14, 16 or 17 positions of the steroid nucleus.

42. An ER ligand having a structure capable of interacting with Leu-384 of ERα or Met-293 of ERβ.

43. An ERα selective ligand capable of interacting with Leu-384 of ERα.

44. An ERβ selective ligand capable of interacting with Met-293 of ERβ.

45. An ERβ selective ligand according to claim 40 further provided with substituents at the 2′ or 3′ positions of the 2-aryl benzothiophene nucleus.

46. An ERβ selective ligand having a substituent larger than a methyl group at the R₂′ position of a 6.3′-dihydroxybenzothiophene.

47. An ERα selective ligand having a substituent larger than a methyl group at either the R₂′ and/or R₃′ position of a 6,5′-dihydroxybenzothiophene.

48. A ligand selective for either ERα or ERβ in which the ligand comprises a position-6 substituent from the benzothiophene nucleus or position-3 substituent from the estradiol nucleus arranged to selectively bind to either the amino acid Ile-326 of ERα or Asn-236 of ERβ.

49. A ligand selective for either ERα or ERβ in which the ligand comprises a position-6 substituent from the benzothiophene nucleus or position-3 substituent from the estradiol nucleus arranged to selectively bind to either the amino acid Phe-445 of ERα or Tyr-354 of ERβ.

50. An ERα selective ligand having a structure capable of simultaneously interacting with Glu-323 and Phe-445 of ERα in preference to Glu-262 and Tyr-354 of ERβ.

51. An ER ligand having a structure arranged to promote binding with Helix H12 of the ER structure.

52. A crystal according to any of claims 1 to 12, having a resolution determined by X-ray crystallography less than 3.5 Å.

53. A machine-readable data storage medium, comprising a data storage material encoded with machine readable data which, when using a machine programmed with instructions for using said data, is capable of displaying a graphical three-dimensional representation of a crystal according to any one of claims 1 to 12 or a homologue of said crystal.

54. A method for evaluating the ability of a chemical entity to associate with an estrogen receptor, the method comprising the steps of:
a) employing computational means to perform a fitting operation between the chemical entity and a binding site of the receptor; and
b) analysing the results of the fitting operation to predict the association between the chemical entity and the binding site.

55. A crystallized molecule or molecular complex comprising a binding pocket defined by the structure coordinates of human ER-α ligand binding domain amino acid residues MET343, LEU346, THR347. LEU349, ALA350, ASP351, GLU353, LEU354, TRP383, LEU384, LEU387, MET388, LEU391, ARG394, PHE404, MET421, ILE424, PHE425, LEU428, GLY521, HIS524, LEU525 or a homologue of said molecule or molecular complex, wherein said homologue has a root mean square deviation from the backbone atoms of said amino acids of not more than 1.5 Å.

56. A homology model comprising a binding pocket defined by the structure coordinates of human ER-β ligand binding domain amino acid residues MET343, LEU346, THR347, LEU349, ALA350, ASP351, GLU353, LEU354, TRP383, MET384, LEU387, MET388, LEU388, LEU391, ARG394, PHE404, ILE421, ILE424, PHE425, LEU428, GLY521, HIS524, LEU525.

57. A crystallized molecule or molecular complex comprising a binding pocket defined by the structure coordinates of rat ER-α ligand binding domain amino acid residues MET252, LEU255, THR256, LEU258, ALA259, ASP260, GLU262, LEU263, TRP292, LEU293, LEU296, MET297, LEU300, ARG303, PHE313, ILE330, IL333, PHE334, LEU337, GLY429, HIS423, LEU433 or a homologue of said molecule or molecular complex, wherein said homologue has a root mean square deviation from the backbone atoms of said amino acids of not more than 1.5 Å.

58. A homology model comprising a binding pocket defined by the structure coordinates of rat ER-β ligand binding domain amino acid residues MET252, LEU255, THR256, LEU258, ALA259, ASP260, GLU262, LEU263, TRP292, MET293, LEU296, MET297, LEU300, ARG303, PHE313, ILE330, ILE333, PHE334, LEU337. GLY429, HIS432, LEU433.

59. A method of agonising or antagonising ERα or ERβ, the method comprising administering to a mammal a compound, other than raloxifene, that fits spatially into the binding pocket of ERβ.

60. A method according to claim 59 in which the compounds has at least one of the following:
a) a group capable of functioning as a hydrogen bond donor to HIS432;
b) A group that functions as a hydrogen bond acceptor and donor to Arg-394 and Glu-353 of ERα or Arg-303 and Glu-262 of ERβ;
c) a group capable of forming a hydrophobic contact with at least one of Met-252, Leu-255, Leu-258, Ala-259, Leu-263, Trp-292, Met-293, Leu-296, Met-297, Leu-300, Phe-313, Ile-330, Ile-333, Phe-334, Leu-337, Leu-433 of ERβ, or Met-343, Leu-346, Leu-349, Ala-350, Leu-354, Trp-383, Leu-384, Leu-387, Met-388, Leu-391, Phe-404, Met-421, Ile-424, Phe-425, Leu-428, Leu-525, of ERα.

61. A method of antagonising ERβ according to claim 59 or 60 in which the compound has a group that can form either a hydrogen bond or a salt bridge to ASP260.

62. A method of antagonising ERα according to claim 59 or 60 in which the compound has a group that can form either a hydrogen bond or a salt bridge to Asp-351.

63. An ER ligand in accordance with any one of the Examples 5 to 55.
